(19) United States
(12) Patent Application Publication
(10) Pub. No.: US 2022/0380776 A1
(43) Pub. Date: Dec. 1, 2022

(54) BASE EDITOR-MEDIATED CD33 REDUCTION TO SELECTIVELY PROTECT THERAPEUTIC CELLS

(71) Applicants: Fred Hutchinson Cancer Center, Seattle, WA (US); University of Washington, Seattle, WA (US)

(72) Inventors: Olivier Humbert, Seattle, WA (US); Hans-Peter Kiem, Seattle, WA (US); Roland B. Walter, Seattle, WA (US); Andre Lieber, Seattle, WA (US); Chang Li, Seattle, WA (US)

(73) Assignees: Fred Hutchinson Cancer Center, Seattle, WA (US); University of Washington, Seattle, WA (US)

(21) Appl. No.: 17/771,128

(22) PCT Filed: Oct. 22, 2020
(86) PCT No.: PCT/US2020/056913

§ 371 Date: Apr. 22, 2022

Related U.S. Application Data
(63) Continuation-in-part of application No. PCT/US2020/040756, filed on Jul. 2, 2020.
(60) Provisional application No. 63/009,385, filed on Apr. 13, 2020.
(60) Provisional application No. 62/935,507, filed on Nov. 14, 2019.
(60) Provisional application No. 62/924,594, filed on Oct. 22, 2019.

(51) Int. Cl. C12N 15/113; C12N 5/00; C12N 5/0789; C12N 9/22

(52) U.S. Cl. CPC C12N 15/1138; C12N 5/0087; C12N 5/0647; C12N 9/22; C12N 2310/20

(57) ABSTRACT

Systems and methods to selectively protect therapeutic cells by reducing CD33 expression in the therapeutic cells using base editors and targeting non-therapeutic cells with an anti-CD33 therapy are described. The selective protection results in the enrichment of the therapeutic cells while simultaneously targeting any diseased, malignant and/or non-therapeutic CD33 expressing cells within a subject.

### CROSS-REFERENCE TO RELATED APPLICATIONS

This application is the 371 National Phase of PCT/2020/056913, filed Oct. 22, 2020, which claims the priority of U.S. Provisional Application No. 62/924,594, filed on Oct. 22, 2019; and is a continuation-in-part (CIP) of PCT/US2020/040756, filed Jul. 2, 2020, which claims the priority of U.S. Provisional Application No. 62/935,507, filed Nov. 14, 2019, and U.S. Provisional Application No. 63/009,385, filed Apr. 13, 2020. The disclosure of each of these earlier-filed applications is hereby incorporated by reference in its entirety.

### STATEMENT REGARDING FEDERALLY SPONSORED RESEARCH OR DEVELOPMENT

This invention was made with government support under contract HL136135 and HL128288 awarded by the National Institutes of Health. The government has certain rights in the invention.

### FIELD OF THE DISCLOSURE

The current disclosure provides systems and methods to selectively protect therapeutic cells by reducing CD33 expression in the therapeutic cells using a base-editing system and subsequently targeting non-therapeutic or unmodified native cells with an anti-CD33 therapy. The selective protection results in the enrichment of the therapeutic cells while simultaneously targeting any diseased, malignant and/or non-therapeutic CD33 expressing cells within a subject.

### BACKGROUND

In various therapeutic and experimental contexts, it can be desirable to selectively eliminate certain cells while selectively protecting others. Hematopoietic stem cells (HSC) are stem cells that can give rise to blood cell types. The therapeutic administration of HSCs can be used to treat a variety of adverse conditions including immune deficiency diseases, non-malignant blood disorders, cancers, infections, and radiation exposure (e.g., cancer treatment, accidental, or attack-based).

### SUMMARY

The present disclosure provides, among other things, methods and compositions related to the use of base editing for selective protection of therapeutic cells from an anti-CD33 agent. In various embodiments, base editing selectively protects therapeutic cells from an anti-CD33 agent by causing a reduction of CD33 expression as compared to a reference.

The current disclosure provides, among other things, systems and methods to selectively protect therapeutic cells by reducing CD33 expression in the therapeutic cells using a base-editing system and subsequently targeting non-therapeutic or unmodified native cells with an anti-CD33 therapy. The selective protection can result in enrichment of therapeutic cells while simultaneously targeting any diseased, malignant and/or non-therapeutic CD33 expressing cells within a subject.

The current disclosure provides systems and methods to protect beneficial therapeutic HSCs from anti-CD33 therapies while leaving residual diseased cells susceptible to anti-CD33 treatments. Various systems and methods achieve this benefit by using base editors (BE) to genetically modify HSC to have reduced or eliminated expression of CD33, thus protecting them from anti-CD33 based therapies. In this manner, genetically modified therapeutic cells will not be harmed by concurrent or subsequent anti-CD33 therapies a patient may receive. However, pre-existing CD33-expressing cells in the patient and/or administered cells that lack the genetic modification will not be protected, resulting in positive selection for the therapeutic cells over other cells. Importantly, use of BE introduces precise nucleotide substitutions and circumvents the need for DNA double strand breaks.

In particular embodiments, the HSC genetically modified to have reduced CD33 expression are also genetically modified for an additional therapeutic purpose. The genetic modification for an additional therapeutic purpose can provide a gene to treat a disorder such as an immune deficiency (e.g., Fanconi anemia, SCID, HIV), a cancer (e.g., leukemia, lymphoma, solid tumor), a blood-related disorder (e.g., sickle cell disease, SCD), a lysosomal storage disease (e.g., Pompe disease, Gaucher disease, Fabry disease, Mucopolysaccharidosis type I), or provide a therapeutic cassette that encodes a chimeric antigen receptor, engineered T-cell receptor, checkpoint inhibitor, or therapeutic antibody.

Methods, systems, and compositions of the present disclosure, which include among other things base editing systems for inactivation of CD33 and uses thereof, are characterized by a number of advantages, both in general and with respect to specific embodiments thereof (e.g., use of particular gRNAs). For example, base editing systems for inactivation of CD33 and uses thereof do not require double stranded breaks in CD33 DNA and for at least that reason are characterized by reduced risk of sequence damage and/or translocation associated, e.g., with CRISPR editing systems. Translocation is particularly problematic in the use of CRISPR when the editing system targets two or more genes or genomic loci and/or when the editing system includes two or more distinct gRNAs, which may lead to intra-chromosomal rearrangement. The present disclosure includes, among other things, embodiments in which a base editing system targets two or more genes or genomic loci and/or in which the base editing system includes two or more distinct gRNAs (e.g., for base editing of a nucleic acid encoding CD33 for CD33 inactivation and base editing of a second nucleic acid, such as where the editing has a therapeutic effect, e.g., increased or decreased expression of a gene or polypeptide of interest).

The genetic modification for an additional therapeutic purpose can provide a gene to treat a disorder, such as a rare hematology indication. Examples of rare hematology indications include, without limitation, rare platelet disorders (e.g. Bernard-Soulier syndrome and Glanzmann thrombasthenia), Bone marrow failure conditions (e.g., Diamond-Blackfan anemia), other red cell disorders (e.g., pyruvate kinase deficiency), autoimmune rare hematologies (e.g., acquired thrombotic thrombocytopenic purpura (aTTP) and congenital thrombotic thrombocytopenic purpura (cTTP)), Primary Immunodeficiencies (PIDs) (e.g., Wiskott-Aldrich syndrome (WAS), Severe combined immunodeficiency due to adenosine deaminase deficiency (ADA-SCID), X-linked severe combined immunodeficiency (SCID-X1), DOCK 8 deficiency, major histocompatibility complex class II deficiency (MHC-II), and CD40/CD40L deficiencies), and other indications that include inborn errors of metabolism (IEMs) (e.g., hereditary hemochromatosis and phenylketonuria (PKU)).

CRISPR editing systems can cause insertion and/or deletion of one or more nucleotides at editing target sites (e.g., sites corresponding to gRNAs of an editing system, e.g., genomic sequences of a targeted and/or edited cell), while in various embodiments base editing systems of the present disclosure do not cause insertion and/or deletion of one or more nucleotides at editing target sites, and/or cause insertion and/or deletion of one or more nucleotide positions at editing target sites at a reduced frequency as compared to a reference CRISPR editing system (e.g., a CRISPR editing system with a same or similar editing target site). In various embodiments, a base editing system of the present disclosure causes insertion and/or deletion of one more nucleotide positions at an editing target site in no more than 0.0001%, 0.001%, 0.01%, 0.1%, 1%, 2%, 3%, 4%, 5%, 10%, 15%, 20%, or 25% of target and/or edited cells, e.g., of a subject or system. In various embodiments, a base editing system of the present disclosure causes insertion and/or deletion of one more nucleotide positions at an editing target site at a frequency that is reduced as compared to a reference CRISPR editing system (e.g., a CRISPR editing system with a same or similar editing target site) by at least 1%, 5%, 10%, 15%, 20%, 30%, 40%, 50%, 75%, 100%, 2-fold, 3-fold, 4-fold, 5-fold, 10-fold, 50-fold, 100-fold, or more. Those of skill in the art will appreciate that a variety of tools and techniques are known in the art of measuring frequency of insertion and/or deletion of nucleotide positions at editing target sites and/or of mutation, including without limitation next generation sequencing. In various embodiments, each of the cells can include a single editing target site. In various embodiments each of the cells can include two or more editing target sites.

In various embodiments, base editing systems of the present disclosure do not cause a DNA emergency repair response, and/or cause a DNA emergency repair response that is reduced in emergency repair response agent activity and/or expression as compared to a reference CRISPR editing system (e.g., a CRISPR editing system with a same or similar editing target site, in a same or similar cell type). In various embodiments, a base editing system of the present disclosure causes a DNA emergency repair response that includes no greater than 10% emergency repair response agent activity and/or expression as compared to a reference CRISPR editing system (e.g., a CRISPR editing system with a same or similar editing target site) (e.g., no greater than 9%, 8%, 7%, 6%, 5%, 4%, 3%, 2%, or 1% emergency repair response agent activity and/or expression as compared to a reference CRISPR editing system, e.g., 1%-10% or 1%-5% emergency repair response agent activity and/or expression as compared to a reference CRISPR editing system).

In various embodiments, percent DNA emergency repair response agent expression is measured as the concentration or amount of DNA emergency repair response polypeptide in a sample from a subject or system as compared to a sample from a reference subject or system. In various embodiments, percent emergency repair response agent expression is measured as the concentration or amount of response agent-encoding messenger RNA in a sample from a subject or system as compared to a sample from a reference subject or system. In various embodiments, an emergency repair response agent is a DNA damage response agent. DNA damage response agents are known to those of skill in the art. In various embodiments, an emergency repair response agent is a DNA damage response agent can be or include without limitation UNG, SMUG1, MBD4, TDG, OGG1, MUTYH (MYH), NTHL1 (NTH1), MPG, NEIL1, NEIL2, NEIL3, APEX1 (APE1), APEX2, LIG3, XRCC1, PNKP, APLF, HMCES, PARP1 (ADPRT), PARP2 (ADPRTL2), PARP3 (ADPRTL3), PARG, PARPBP, MGMT, ALKBH2 (ABH2), ALKBH3 (DEPC1), TDP1, TDP2 (TTRAP), SPRTN (Spartan), MSH2, MSH3, MSH6, MLH1, PMS2, MSH4, MSH5, MLH3, PMS1, PMS2P3 (PMS2L3), HFM1, XPC, RAD23B, CETN2, RAD23A, XPA, DDB1, DDB2 (XPE), RPA1, RPA2, RPA3, TFIIHERCC3 (XPB), ERCC2 (XPD), GTF2H1, GTF2H2, GTF2H3, GTF2H4, GTF2H5 (TTDA), GTF2E2, CDK7, CCNH, MNAT1, ERCC5 (XPG), ERCC1, ERCC4 (XPF), LIG1, ERCC8 (CSA), ERCC6 (CSB), UVSSA (KIAA1530), XAB2 (HCNP), MMS19, RAD51, RAD51B, RAD51D, HELQ (HEL308), SWI5, SWSAP1, ZSWIM7 (SWS1), SPIDR, PDS5B, DMC1, XRCC2, XRCC3, RAD52, RAD54L, RAD54B, BRCA1, BARD1, ABRAXAS1, PAXIP1 (PTIP), SMC5, SMC6, SHLD1, SHLD2 (FAM35A), SHLD3, SEM1 (SHFM1) (DSS1), RAD50, MRE11A, NBN (NBS1), RBBP8 (CtIP), MUS81, EME1 (MMS4L), EME2, SLX1A (GIYD1), SLX1B (GIYD2), GEN1, FANCA, FANCB, FANCC, BRCA2 (FANCD1), FANCD2, FANCE, FANCF, FANCG (XRCC9), FANCI (KIAA1794), BRIP1 (FANCJ), FANCL, FANCM, PALB2 (FANCN), RAD51C (FANCO), SLX4(FANCP), FAAP20 (C1orf86), FAAP24 (C19orf40), FAAP100, UBE2T (FANCT), XRCC6 (Ku70), XRCCS (Ku80), PRKDC, LIG4, XRCC4, DCLRE1C (Artemis), NHEJ1 (XLF, Cernunnos), NUDT1 (MTH1), DUT, RRM2B (p53R2), PARK7 (DJ-1), DNPH1, NUDT15 (MTH2), NUDT18 (MTH3), POLA1, POLB, POLD1, POLD2, POLD3, POLD4, POLE (POLE1), POLE2, POLE3, POLE4, REV3L (POLZ), MAD2L2 (REV7), REV1 (REV1L), POLG, POLH, POLI (RAD30B), POLQ, POLK (DINB1), POLL, POLM, POLN (POL4P), PRIMPOL, DNTT, FEN1 (DNase IV), FAN1 (MTMR15), TREX1, TREX2, EXO1 (HEX1), APTX (aprataxin), SPO11, ENDOV, DNA2, DCLRE1A (SNM1A), DCLRE1B (SNM1B), EXOS, UBE2A (RAD6A), UBE2B (RAD6B), RAD18, SHPRH, HLTF (SMARCA3), RNF168, RNF8, RNF4, UBE2V2 (MMS2), UBE2N (UBC13), USP1, WDR48, HERC2, H2AX (H2AFX), CHAF1A (CAF1), SETMAR (METNASE), ATRX, BLM, RMI1, TOP3A, WRN, RECQL4, ATM, MPLKIP (TTDN1), RPA4, PRPF19 (PSO4), RECQL (RECQ1), RECQLS, RDM1 (RAD52B), NABP2 (SSB1), ATR, ATRIP, MDC1, PCNA, RAD1, RAD9A, HUS1, RAD17 (RAD24), CHEK1, CHEK2, TP53, TP53BP1 (53BP1), RIF1, TOPBP1, CLK2, and/or PER1.

In particular embodiments, the systems and methods described herein further provide systems and methods to reduce or eliminate the need for genotoxic conditioning. Currently, conditioning is used to remove a patient's existing hematopoietic system. All of the currently used conditioning regimens, however, whether myeloablative or nonmyeloablative, rely on the use of alkylating chemotherapy drugs and/or radiation such as involve total body irradiation (TBI) and/or cytotoxic drugs. Aside from any potential remaining residual cells, these conditioning regimens are also independently associated with an increased risk of developing malignancies, especially in DNA repair disorders like FA. These regimens are non-targeted, genotoxic, and have multiple short- and long-term adverse effects (La Nasa et al., Bone Marrow Transplant 36:971-975, 2005 and Chen et al., Blood. 107:3764-3771, 2006) such as an increased risk of developing DNA repair disorders, interstitial pneumonitis, idiopathic pulmonary fibrosis, reduced lung pulmonary function, renal damage, sinusoidal obstruction syndrome (SOS), infertility, cataract formation, hyperthyroidism, and thyroiditis (Gyurkocza et al., Blood. 124:344-353, 2014). Not only do these regimens result in impaired immune function, but they are associated with significant morbidity and mortality (Armitage, N Engl J Med. 1996; 330:827-837). Therefore, methods to reduce or eliminate the need for conditioning in these patients are desperately needed. In particular embodiments, the systems and methods allow the targeting and removal of any remaining CD33-expressing cells following conditioning in preparation for a hematopoietic cell transplant, bone marrow transplant, and/or administration of therapeutic cells (e.g., genetically-modified therapeutic cells). In particular embodiments, the systems and methods clear the bone marrow niche and allow for further expansion of gene-corrected cells. In particular embodiments, the systems and methods deplete residual disease-related cells. The therapeutically administered cells with reduced CD33 expression are protected from the CD33-targeting and are able to reconstitute the patient's blood and immune systems.

Thus, the systems and methods provide a selective protective advantage to the genetically modified cells as they reconstitute the patient's blood and immune systems while also allowing the continued use of anti-CD33 therapies to target remaining, diseased and/or malignant CD33-expressing cells within a subject as well as any administered cells lacking the intended genetic modification. In combination, the approaches disclosed herein can eliminate CD33-expressing cells, resulting in a completely gene-corrected hematopoiesis, and minimizing risks of future myeloid malignancy after gene therapy or allogeneic transplantation.

The base editors introduce precise nucleotide substitutions and circumvent the need for DNA double strand breaks. As described herein, different strategies for introducing non-sense and splicing mutations in CD33 were investigated. BE-treatment of human CD34+ HSPCs did not impair engraftment and differentiation in a mouse model, while reducing CD33 expression and protecting cells from in vivo gemtuzumab ozogamicin (GO) administration. Next-generation sequencing analysis of blood nucleated cells confirmed the persistence and specificity of BE-induced mutations in vivo. Together, the results validate the use of BE for the generation of CD33-engineered hematopoiesis to improve safety and efficacy of CD33-targeted therapies. BE can also target multiple sites simultaneously and can be used for in vivo selection of gene modified cells using CD33-directed immunotherapies. Thus, the systems and methods disclosed herein can be used to improve therapies involving blood bone marrow transplant (BMT), autologous cell therapies, and treatments for diseases associated with cellular expression of CD33.

### Definitions

A, An, The: As used herein, “a”, “an”, and “the” refer to one or to more than one (i.e., to at least one) of the grammatical object of the article. By way of example, “an element” discloses embodiments of exactly one element and embodiments including more than one element.

About: As used herein, term “about”, when used in reference to a value, refers to a value that is similar, in context to the referenced value. In general, those skilled in the art, familiar with the context, will appreciate the relevant degree of variance encompassed by “about” in that context. For example, in some embodiments, the term “about” may encompass a range of values that within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referenced value.

Administration: As used herein, the term “administration” typically refers to administration of a composition to a subject or system to achieve delivery of an agent that is, or is included in, the composition.

Agent: As used herein, the term “agent” may refer to any chemical entity, including without limitation any of one or more of an atom, molecule, compound, amino acid, polypeptide, nucleotide, nucleic acid, protein, protein complex, liquid, solution, saccharide, polysaccharide, lipid, or combination or complex thereof.

Between or From: As used herein, the term “between” refers to content that falls between indicated upper and lower, or first and second, boundaries, inclusive of the boundaries. Similarly, the term “from”, when used in the context of a range of values, indicates that the range includes content that falls between indicated upper and lower, or first and second, boundaries, inclusive of the boundaries.

Binding: As used herein, the term “binding” refers to a non-covalent association between or among two or more agents. “Direct” binding involves physical contact between agents; indirect binding involves physical interaction by way of physical contact with one or more intermediate agents. Binding between two or more agents can occur and/or be assessed in any of a variety of contexts, including where interacting agents are studied in isolation or in the context of more complex systems (e.g., while covalently or otherwise associated with a carrier agents and/or in a biological system or cell).

Cancer: As used herein, the term “cancer” refers to a condition, disorder, or disease in which cells exhibit relatively abnormal, uncontrolled, and/or autonomous growth, so that they display an abnormally elevated proliferation rate and/or aberrant growth phenotype characterized by a significant loss of control of cell proliferation. In some embodiments, a cancer can include one or more tumors. In some embodiments, a cancer can be or include cells that are precancerous (e.g., benign), malignant, pre-metastatic, metastatic, and/or non-metastatic. In some embodiments, a cancer can be or include a solid tumor. In some embodiments, a cancer can be or include a hematologic tumor.

Chimeric antigen receptor: As used herein, “Chimeric antigen receptor” or “CAR” refers to an engineered protein that includes (i) an extracellular domain that includes a moiety that binds a target antigen; (ii) a transmembrane domain; and (iii) an intracellular signaling domain that sends activating signals when the CAR is stimulated by binding of the extracellular binding moiety with a target antigen. A T cell that has been genetically engineered to express a chimeric antigen receptor may be referred to as a CAR T cell. Thus, for example, when certain CARs are expressed by a T cell, binding of the CAR extracellular binding moiety with a target antigen can activate the T cell. CARs are also known as chimeric T cell receptors or chimeric immunoreceptors.

Combination therapy: As used herein, the term “combination therapy” refers to administration to a subject of to two or more agents or regimens such that the two or more agents or regimens together treat a condition, disorder, or disease of the subject. In some embodiments, the two or more agents or regimens can be administered simultaneously, sequentially, or in overlapping dosing regimens. Those of skill in the art will appreciate that combination therapy includes but does not require that the two agents or regimens be administered together in a single composition, nor at the same time.

Control expression or activity: As used herein, a first element (e.g., a protein, such as a transcription factor, or a nucleic acid sequence, such as promoter) “controls” or “drives” expression or activity of a second element (e.g., a protein or a nucleic acid encoding an agent such as a protein) if the expression or activity of the second element is wholly or partially dependent upon status (e.g., presence, absence, conformation, chemical modification, interaction, or other activity) of the first under at least one set of conditions. Control of expression or activity can be substantial control or activity, e.g., in that a change in status of the first element can, under at least one set of conditions, result in a change in expression or activity of the second element of at least 10% (e.g., at least 20%, 30%, 40%, 50%, 60%, 70%, 80%, 90%, 100%, 2-fold, 3-fold, 4-fold, 5-fold, 10-fold, 20-fold, 30-fold, 40-fold, 50-fold, 100-fold) as compared to a reference control.

Corresponding to: As used herein, the term “corresponding to” may be used to designate the position/identity of a structural element in a compound or composition through comparison with an appropriate reference compound or composition. For example, in some embodiments, a monomeric residue in a polymer (e.g., an amino acid residue in a polypeptide or a nucleic acid residue in a polynucleotide) may be identified as “corresponding to” a residue in an appropriate reference polymer. For example, those of skill in the art appreciate that residues in a provided polypeptide or polynucleotide sequence are often designated (e.g., numbered or labeled) according to the scheme of a related reference sequence (even if, e.g., such designation does not reflect literal numbering of the provided sequence). By way of illustration, if a reference sequence includes a particular amino acid motif at positions 100-110, and a second related sequence includes the same motif at positions 110-120, the motif positions of the second related sequence can be said to “correspond to” positions 100-110 of the reference sequence. Those of skill in the art appreciate that corresponding positions can be readily identified, e.g., by alignment of sequences, and that such alignment is commonly accomplished by any of a variety of known tools, strategies, and/or algorithms, including without limitation software programs such as, for example, BLAST, CS-BLAST, CUDASW++, DIAMOND, FASTA, GGSEARCH/GLSEARCH, Genoogle, HMMER, HHpred/HHsearch, IDF, Infernal, KLAST, USEARCH, parasail, PSI-BLAST, PSI-Search, ScalaBLAST, Sequilab, SAM, SSEARCH, SWAPHI, SWAPHI-LS, SWIMM, or SWIPE.

Dosing regimen: As used herein, the term “dosing regimen” can refer to a set of one or more same or different unit doses administered to a subject, typically including a plurality of unit doses, administration of each of which is separated from administration of the others by a period of time. In various embodiments, one or more or all unit doses of a dosing regimen may be the same or can vary (e.g., increase over time, decrease over time, or be adjusted in accordance with the subject and/or with a medical practitioner's determination). In various embodiments, one or more or all of the periods of time between each dose may be the same or can vary (e.g., increase over time, decrease over time, or be adjusted in accordance with the subject and/or with a medical practitioner's determination). In some embodiments, a given therapeutic agent has a recommended dosing regimen, which can involve one or more doses. Typically, at least one recommended dosing regimen of a marketed drug is known to those of skill in the art. In some embodiments, a dosing regimen is correlated with a desired or beneficial outcome when administered across a relevant population (i.e., is a therapeutic dosing regimen).

Downstream and Upstream: As used herein, the term“downstream” means that a first DNA region is closer, relative to a second DNA region, to the C-terminus of a nucleic acid that includes the first DNA region and the second DNA region. As used herein, the term “upstream” means a first DNA region is closer, relative to a second DNA region, to the N-terminus of a nucleic acid that includes the first DNA region and the second DNA region.

Effective amount: An “effective amount” is the amount of a formulation necessary to result in a desired physiological change in a subject. Effective amounts are often administered for research purposes.

Engineered: As used herein, the term “engineered” refers to the aspect of having been manipulated by the hand of man. For example, a polynucleotide is considered to be “engineered” when two or more sequences, that are not linked together in that order in nature, are manipulated by the hand of man to be directly linked to one another in the engineered polynucleotide. Those of skill in the art will appreciate that an “engineered” nucleic acid or amino acid sequence can be a recombinant nucleic acid or amino acid sequence, and can be referred to as “genetically engineered.” In some embodiments, an engineered polynucleotide includes a coding sequence and/or a regulatory sequence that is found in nature operably linked with a first sequence but is not found in nature operably linked with a second sequence, which is in the engineered polynucleotide operably linked in with the second sequence by the hand of man. In some embodiments, a cell or organism is considered to be “engineered” or “genetically engineered” if it has been manipulated so that its genetic information is altered (e.g., new genetic material not previously present has been introduced, for example by transformation, mating, somatic hybridization, transfection, transduction, or other mechanism, or previously present genetic material is altered or removed, for example by substitution, deletion, or mating). As is common practice and is understood by those of skill in the art, progeny or copies, perfect or imperfect, of an engineered polynucleotide or cell are typically still referred to as “engineered” even though the direct manipulation was of a prior entity.

Excipient: As used herein, “excipient” refers to a non-therapeutic agent that may be included in a pharmaceutical composition, for example to provide or contribute to a desired consistency or stabilizing effect. In some embodiments, suitable pharmaceutical excipients may include, for example, starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, or the like.

Expression: As used herein, “expression” refers individually and/or cumulatively to one or more biological process that result in production from a nucleic acid sequence of an encoded agent, such as a protein. Expression specifically includes either or both of transcription and translation.

Fragment: As used herein, “fragment” refers a structure that includes and/or consists of a discrete portion of a reference agent (sometimes referred to as the “parent” agent). In some embodiments, a fragment lacks one or more moieties found in the reference agent. In some embodiments, a fragment includes or consists of one or more moieties found in the reference agent. In some embodiments, the reference agent is a polymer such as a polynucleotide or polypeptide. In some embodiments, a fragment of a polymer includes or consists of at least 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 275, 300, 325, 350, 375, 400, 425, 450, 475, 500 or more monomeric units (e.g., residues) of the reference polymer. In some embodiments, a fragment of a polymer includes or consists of at least 5%, 10%, 15%, 20%, 25%, 30%, 25%, 40%, 45%, 50%, 55%, 60%, 65%, 70%, 75%, 80%, 85%, 90%, 95%, 96%, 97%, 98%, 99% or more of the monomeric units (e.g., residues) found in the reference polymer. A fragment of a reference polymer is not necessarily identical to a corresponding portion of the reference polymer. For example, a fragment of a reference polymer can be a polymer having a sequence of residues having at least 5%, 10%, 15%, 20%, 25%, 30%, 25%, 40%, 45%, 50%, 55%, 60%, 65%, 70%, 75%, 80%, 85%, 90%, 95%, 96%, 97%, 98%, 99% or more identity to the reference polymer. A fragment may, or may not, be generated by physical fragmentation of a reference agent. In some instances, a fragment is generated by physical fragmentation of a reference agent. In some instances, a fragment is not generated by physical fragmentation of a reference agent and can be instead, for example, produced by de novo synthesis or other means.

The term “gene” refers to a nucleic acid sequence (in various instances used interchangeably with polynucleotide or nucleotide sequence) that includes a coding sequence that encodes a therapeutic sequence, protein, or other expression product (such as an RNA product and/or a polypeptide product) as described herein, optionally together with some or all of regulatory sequences that control expression of the coding sequence. Gene sequences encoding a molecule can be DNA or RNA. As appropriate for the given context, these nucleic acid sequences may be a DNA strand sequence that is transcribed into RNA or an RNA sequence that is translated into protein. The nucleic acid sequences include both the full-length nucleic acid sequences as well as non-full-length sequences derived from the full-length protein. The gene sequence can be readily prepared by synthetic or recombinant methods. The definition of a gene includes various sequence polymorphisms; mutations; degenerate codons of the native sequence; sequences that may be introduced to provide codon preference in a specific cell type (e.g., codon optimized for expression in mammalian cells); and/or sequence variants wherein such alterations do not substantially affect the function of the encoded molecule. The term further can include all introns and other DNA sequences spliced from an mRNA transcript, along with variants resulting from alternative splice sites. Portions of complete gene sequences are referenced throughout the disclosure as is understood by one of ordinary skill in the art. Nucleotide sequences encoding other sequences disclosed herein can be readily determined by one of ordinary skill in the art. The term “gene” may include not only coding sequences but also coding sequences operably linked to each other and relevant regulatory sequences such as promoters, enhancers, and termination regions. For example, there can be a functional linkage between a regulatory sequence and an exogenous nucleic acid sequence resulting in expression of the latter. For another example, a first nucleic acid sequence can be operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary or helpful, join coding regions into a common reading frame. In some embodiments, a gene includes non-coding sequence such as, without limitation, introns. In some embodiments, a gene may include both coding (e.g., exonic) and non-coding (e.g., intronic) sequences. In some embodiments, a gene includes a regulatory sequence that is a promoter. In some embodiments, a gene includes one or both of a (i) DNA nucleotides extending a predetermined number of nucleotides upstream of the coding sequence in a reference context, such as a source genome, and (ii) DNA nucleotides extending a predetermined number of nucleotides downstream of the coding sequence in a reference context, such as a source genome. In various embodiments, the predetermined number of nucleotides can be 500 bp, 1 kb, 2 kb, 3 kb, 4 kb, 5 kb, 10 kb, 20 kb, 30 kb, 40 kb, 50 kb, 75 kb, or 100 kb. As used herein, a “transgene” refers to a gene that is not endogenous or native to a reference context in which the gene is present or into which the gene may be placed by engineering.

Gene product or expression product: As used herein, the term “gene product” or “expression product” generally refers to an RNA transcribed from the gene (pre- and/or post-processing) or a polypeptide (pre- and/or post-modification) encoded by an RNA transcribed from the gene.

Host cell, target cell: As used herein, “host cell” refers to a cell into which exogenous DNA (recombinant or otherwise), such as a transgene, has been introduced. Those of skill in the art appreciate that a “host cell” can be the cell into which the exogenous DNA was initially introduced and/or progeny or copies, perfect or imperfect, thereof. In some embodiments, a host cell includes one or more viral genes or transgenes. In some embodiments, an intended or potential host cell can be referred to as a target cell.

In various embodiments, a host cell or target cell is identified by the presence, absence, or expression level of various surface markers.

A statement that a cell or population of cells is “positive” for or expressing a particular marker refers to the detectable presence on or in the cell of the particular marker. When referring to a surface marker, the term can refer to the presence of surface expression as detected by flow cytometry, for example, by staining with an antibody that specifically binds to the marker and detecting said antibody, wherein the staining is detectable by flow cytometry at a level substantially above the staining detected carrying out the same procedure with an isotype-matched control under otherwise identical conditions and/or at a level substantially similar to that for cell known to be positive for the marker, and/or at a level substantially higher than that for a cell known to be negative for the marker.

A statement that a cell or population of cells is “negative” for a particular marker or lacks expression of a marker refers to the absence of substantial detectable presence on or in the cell of a particular marker. When referring to a surface marker, the term can refer to the absence of surface expression as detected by flow cytometry, for example, by staining with an antibody that specifically binds to the marker and detecting said antibody, wherein the staining is not detected by flow cytometry at a level substantially above the staining detected carrying out the same procedure with an isotype-matched control under otherwise identical conditions, and/or at a level substantially lower than that for cell known to be positive for the marker, and/or at a level substantially similar as compared to that for a cell known to be negative for the marker.

Identity: As used herein, the term “identity” refers to the overall relatedness between polymeric molecules, e.g., between nucleic acid molecules (e.g., DNA molecules and/or RNA molecules) and/or between polypeptide molecules. “% sequence identity” can refer to a relationship between two or more sequences, as determined by comparing the sequences. Methods for the calculation of a percent identity as between two provided sequences are known in the art. The term “% sequence identity” refers to a relationship between two or more sequences, as determined by comparing the sequences. In the art, “identity” also means the degree of sequence relatedness between protein, nucleic acid, or gene sequences as determined by the match between strings of such sequences. “Identity” (often referred to as “similarity”) can be readily calculated by known methods, including those described in: Computational Molecular Biology (Lesk, A. M., ed.) Oxford University Press, N Y (1988); Biocomputing: Informatics and Genome Projects (Smith, D. W., ed.) Academic Press, N Y (1994); Computer Analysis of Sequence Data, Part I (Griffin, A. M., and Griffin, H. G., eds.) Humana Press, N J (1994); Sequence Analysis in Molecular Biology (Von Heijne, G., ed.) Academic Press (1987); and Sequence Analysis Primer (Gribskov, M. and Devereux, J., eds.) Oxford University Press, NY (1992). Preferred methods to determine identity are designed to give the best match between the sequences tested. Methods to determine identity and similarity are codified in publicly available computer programs. For instance, calculation of the percent identity of two nucleic acid or polypeptide sequences, for example, can be performed by aligning the two sequences (or the complement of one or both sequences) for optimal comparison purposes (e.g., gaps can be introduced in one or both of a first and a second sequences for optimal alignment and non-identical sequences can be disregarded for comparison purposes). The nucleotides or amino acids at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, optionally accounting for the number of gaps, and the length of each gap, which may need to be introduced for optimal alignment of the two sequences. The comparison of sequences and determination of percent identity between two sequences can be accomplished using a computational algorithm, such as BLAST (basic local alignment search tool). Sequence alignments and percent identity calculations may be performed using the Megalign program of the LASERGENE bioinformatics computing suite (DNASTAR, Inc., Madison, Wis.). Multiple alignment of the sequences can also be performed using the Clustal method of alignment (Higgins and Sharp CABIOS, 5, 151-153 (1989) with default parameters (GAP PENALTY=10, GAP LENGTH PENALTY=10). Relevant programs also include the GCG suite of programs (Wisconsin Package Version 9.0, Genetics Computer Group (GCG), Madison, Wis.); BLASTP, BLASTN, BLASTX (Altschul et al., J. Mol. Biol. 215:403-410 (1990); DNASTAR (DNASTAR, Inc., Madison, Wis.); and the FASTA program incorporating the Smith-Waterman algorithm (Pearson, Comput. Methods Genome Res., [Proc. Int. Symp.] (1994), Meeting Date 1992, 111-20. Editor(s): Suhai, Sandor. Publisher: Plenum, New York, N.Y. Within the context of this disclosure it will be understood that where sequence analysis software is used for analysis, the results of the analysis are based on the “default values” of the program referenced. As used herein “default values” will mean any set of values or parameters, which originally load with the software when first initialized.

“Improve,” “increase,” “inhibit,” or “reduce”: As used herein, the terms “improve”, “increase”, “inhibit”, and “reduce”, and grammatical equivalents thereof, indicate qualitative or quantitative difference from a reference.

Isolated: As used herein, “isolated” refers to a substance and/or entity that has been (1) separated from at least some of the components with which it was associated when initially produced (whether in nature and/or in an experimental setting), and/or (2) designed, produced, prepared, and/or manufactured by the hand of man. Isolated substances and/or entities may be separated from 10%, 20%, 30%, 40%, 50%, 60%, 70%, 80%, 90%, 91%, 92%, 93%, 94%, 95%, 96%, 97%, 98%, 99%, or more than 99% of the other components with which they were initially associated. In some embodiments, isolated agents are 80%, 85%, 90%, 91%, 92%, 93%, 94%, 95%, 96%, 97%, 98%, 99%, or more than 99% pure. As used herein, a substance is “pure” if it is substantially free of other components. In some embodiments, as will be understood by those skilled in the art, a substance may still be considered “isolated” or even “pure”, after having been combined with certain other components such as, for example, one or more carriers or excipients (e.g., buffer, solvent, water, etc.); in such embodiments, percent isolation or purity of the substance is calculated without including such carriers or excipients. To give but one example, in some embodiments, a biological polymer such as a polypeptide or polynucleotide that occurs in nature is considered to be “isolated” when, a) by virtue of its origin or source of derivation is not associated with some or all of the components that accompany it in its native state in nature; b) it is substantially free of other polypeptides or nucleic acids of the same species from the species that produces it in nature; c) is expressed by or is otherwise in association with components from a cell or other expression system that is not of the species that produces it in nature. Thus, for instance, in some embodiments, a polypeptide that is chemically synthesized or is synthesized in a cellular system different from that which produces it in nature is considered to be an “isolated” polypeptide. Alternatively or additionally, in some embodiments, a polypeptide that has been subjected to one or more purification techniques may be considered to be an “isolated” polypeptide to the extent that it has been separated from other components a) with which it is associated in nature; and/or b) with which it was associated when initially produced.

Operably linked: As used herein, “operably linked” or “operatively linked” refers to the association of at least a first element and a second element such that the component elements are in a relationship permitting them to function in their intended manner. For example, a nucleic acid regulatory sequence is “operably linked” to a nucleic acid coding sequence if the regulatory sequence and coding sequence are associated in a manner that permits control of expression of the coding sequence by the regulatory sequence. In some embodiments, an “operably linked” regulatory sequence is directly or indirectly covalently associated with a coding sequence (e.g., in a single nucleic acid). In some embodiments, a regulatory sequence controls expression of a coding sequence in trans and inclusion of the regulatory sequence in the same nucleic acid as the coding sequence is not a requirement of operable linkage.

Pharmaceutically acceptable: As used herein, the term “pharmaceutically acceptable,” as applied to one or more, or all, component(s) for formulation of a composition as disclosed herein, means that each component must be compatible with the other ingredients of the composition and not deleterious to the recipient thereof.

Pharmaceutically acceptable carrier: As used herein, the term “pharmaceutically acceptable carrier” refers to a pharmaceutically-acceptable material, composition, or vehicle, such as a liquid or solid filler, diluent, excipient, or solvent encapsulating material, that facilitates formulation of an agent (e.g., a pharmaceutical agent), modifies bioavailability of an agent, or facilitates transport of an agent from one organ or portion of a subject to another. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

Pharmaceutical composition: As used herein, the term “pharmaceutical composition” refers to a composition in which an active agent is formulated together with one or more pharmaceutically acceptable carriers.

Promoter: As used herein, a “promoter” or “promoter sequence” can be a DNA regulatory region that directly or indirectly (e.g., through promoter-bound proteins or substances) participates in initiation and/or processivity of transcription of a coding sequence. A promoter may, under suitable conditions, initiate transcription of a coding sequence upon binding of one or more transcription factors and/or regulatory moieties with the promoter. A promoter that participates in initiation of transcription of a coding sequence can be “operably linked” to the coding sequence. In certain instances, a promoter can be or include a DNA regulatory region that extends from a transcription initiation site (at its 3′ terminus) to an upstream (5′ direction) position such that the sequence so designated includes one or both of a minimum number of bases or elements necessary to initiate a transcription event. A promoter may be, include, or be operably associated with or operably linked to, expression control sequences such as enhancer and repressor sequences. In some embodiments, a promoter may be inducible. In some embodiments, a promoter may be a constitutive promoter. In some embodiments, a conditional (e.g., inducible) promoter may be unidirectional or bi-directional. A promoter may be or include a sequence identical to a sequence known to occur in the genome of particular species. In some embodiments, a promoter can be or include a hybrid promoter, in which a sequence containing a transcriptional regulatory region can be obtained from one source and a sequence containing a transcription initiation region can be obtained from a second source. Systems for linking control elements to coding sequence within a transgene are well known in the art (general molecular biological and recombinant DNA techniques are described in Sambrook, Fritsch, and Maniatis, Molecular Cloning: A Laboratory Manual, Second Edition, Cold Spring Harbor Laboratory Press, Cold Spring Harbor, N.Y., 1989).

Reference: As used herein, “reference” refers to a standard or control relative to which a comparison is performed. For example, in some embodiments, an agent, sample, sequence, subject, animal, or individual, or population thereof, or a measure or characteristic representative thereof, is compared with a reference, an agent, sample, sequence, subject, animal, or individual, or population thereof, or a measure or characteristic representative thereof. In some embodiments, a reference is a measured value. In some embodiments, a reference is an established standard or expected value. In some embodiments, a reference is a historical reference. A reference can be quantitative of qualitative. Typically, as would be understood by those of skill in the art, a reference and the value to which it is compared represents measure under comparable conditions. Those of skill in the art will appreciate when sufficient similarities are present to justify reliance on and/or comparison. In some embodiments, an appropriate reference may be an agent, sample, sequence, subject, animal, or individual, or population thereof, under conditions those of skill in the art will recognize as comparable, e.g., for the purpose of assessing one or more particular variables (e.g., presence or absence of an agent or condition), or a measure or characteristic representative thereof.

Obtained values for parameters associated with a therapy described herein can be compared to a reference level derived from a control population, and this comparison can indicate whether a therapy described herein is effective for a subject in need thereof. Reference levels can be obtained from one or more relevant datasets from a control population. A “dataset” as used herein is a set of numerical values resulting from evaluation of a sample (or population of samples) under a desired condition. The values of the dataset can be obtained, for example, by experimentally obtaining measures from a sample and constructing a dataset from these measurements. As is understood by one of ordinary skill in the art, the reference level can be based on e.g., any mathematical or statistical formula useful and known in the art for arriving at a meaningful aggregate reference level from a collection of individual data points; e.g., mean, median, median of the mean, etc. Alternatively, a reference level or dataset to create a reference level can be obtained from a service provider such as a laboratory, or from a database or a server on which the dataset has been stored.

A reference level from a dataset can be derived from previous measures derived from a control population. A “control population” is any grouping of subjects or samples of like specified characteristics. The grouping could be according to, for example, clinical parameters, clinical assessments, therapeutic regimens, disease status, severity of condition, etc. In particular embodiments, the grouping is based on age range (e.g., 0-2 years) and non-immunocompromised status. In particular embodiments, a normal control population includes individuals that are age-matched to a test subject and non-immune compromised. In particular embodiments, age-matched includes, e.g., 0-6 months old; 0-2 years old; 0-10 years old; 10-15 years old, 60-65 years old, 70-85 years old, etc., as is clinically relevant under the circumstances. In particular embodiments, a control population can include those that have an immune deficiency and have not been administered a therapeutically effective amount

In particular embodiments, the relevant reference level for values of a particular parameter associated with a therapy described herein is obtained based on the value of a particular corresponding parameter associated with a therapy in a control population to determine whether a therapy disclosed herein has been therapeutically effective for a subject in need thereof.

In particular embodiments, conclusions are drawn based on whether a sample value is statistically significantly different or not statistically significantly different from a reference level. A measure is not statistically significantly different if the difference is within a level that would be expected to occur based on chance alone. In contrast, a statistically significant difference or increase is one that is greater than what would be expected to occur by chance alone. Statistical significance or lack thereof can be determined by any of various methods well-known in the art. An example of a commonly used measure of statistical significance is the p-value. The p-value represents the probability of obtaining a given result equivalent to a particular data point, where the data point is the result of random chance alone. A result is often considered significant (not random chance) at a p-value less than or equal to 0.05. In particular embodiments, a sample value is “comparable to” a reference level derived from a normal control population if the sample value and the reference level are not statistically significantly different.

Regulatory sequence: As used herein in the context of expression of a nucleic acid coding sequence, a regulatory sequence is a nucleic acid sequence that controls expression of a coding sequence. In some embodiments, a regulatory sequence can control or impact one or more aspects of gene expression (e.g., cell-type-specific expression, inducible expression, etc.).

Subject: As used herein, the term “subject” refers to an organism, typically a mammal (e.g., a human, rat, or mouse). In some embodiments, a subject is suffering from a disease, disorder or condition. In some embodiments, a subject is susceptible to a disease, disorder, or condition. In some embodiments, a subject displays one or more symptoms or characteristics of a disease, disorder or condition. In some embodiments, a subject is not suffering from a disease, disorder or condition. In some embodiments, a subject does not display any symptom or characteristic of a disease, disorder, or condition. In some embodiments, a subject has one or more features characteristic of susceptibility to or risk of a disease, disorder, or condition. In some embodiments, a subject is a subject that has been tested for a disease, disorder, or condition, and/or to whom therapy has been administered. In some instances, a human subject can be interchangeably referred to as a “patient” or “individual.”

Therapeutic agent: As used herein, the term “therapeutic agent” refers to any agent that elicits a desired pharmacological effect when administered to a subject. In some embodiments, an agent is considered to be a therapeutic agent if it demonstrates a statistically significant effect across an appropriate population. In some embodiments, the appropriate population can be a population of model organisms or a human population. In some embodiments, an appropriate population can be defined by various criteria, such as a certain age group, gender, genetic background, preexisting clinical conditions, etc. In some embodiments, a therapeutic agent is a substance that can be used for treatment of a disease, disorder, or condition. In some embodiments, a therapeutic agent is an agent that has been or is required to be approved by a government agency before it can be marketed for administration to humans. In some embodiments, a therapeutic agent is an agent for which a medical prescription is required for administration to humans.

Therapeutically effective amount: As used herein, “therapeutically effective amount” or an “effective amount” refers to an amount of an agent or formulation necessary or sufficient to result in a desired physiological change in a subject or population. Effective amounts are often administered for research purposes. In some embodiments, a therapeutically effective amount is one that reduces the incidence and/or severity of, and/or delays onset of, one or more symptoms of the disease, disorder, and/or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment. In some embodiments, reference to a therapeutically effective amount may be a reference to an amount as measured in one or more specific tissues (e.g., a tissue affected by the disease, disorder or condition) or fluids (e.g., blood, saliva, serum, sweat, tears, urine, etc.). Those of ordinary skill in the art will appreciate that, in some embodiments, a therapeutically effective amount of a particular agent or therapy may be formulated and/or administered in a single dose. In some embodiments, a therapeutically effective agent may be formulated and/or administered in a plurality of doses, for example, as part of a dosing regimen.

Treatment: As used herein, the term “treatment” (also “treat” or “treating”) refers to administration of a therapy that partially or completely alleviates, ameliorates, relieves, inhibits, delays onset of, reduces severity of, and/or reduces incidence of one or more symptoms, features, and/or causes of a particular disease, disorder, or condition, or is administered for the purpose of achieving any such result. In some embodiments, treatment can be of a subject who does not exhibit signs of the relevant disease, disorder, or condition and/or of a subject who exhibits only early signs of the disease, disorder, or condition. Alternatively or additionally, such treatment can be of a subject who exhibits one or more established signs of the relevant disease, disorder and/or condition. In some embodiments, treatment can be of a subject who has been diagnosed as suffering from the relevant disease, disorder, and/or condition. In some embodiments, treatment can be of a subject known to have one or more susceptibility factors that are statistically correlated with increased risk of development of the relevant disease, disorder, or condition. A “prophylactic treatment” includes a treatment administered to a subject who does not display signs or symptoms of a condition to be treated or displays only early signs or symptoms of the condition to be treated such that treatment is administered for the purpose of diminishing, preventing, or decreasing the risk of developing the condition. Thus, a prophylactic treatment functions as a preventative treatment against a condition. A “therapeutic treatment” includes a treatment administered to a subject who displays symptoms or signs of a condition and is administered to the subject for the purpose of reducing the severity or progression of the condition.

Unit dose: As used herein, the term “unit dose” refers to an amount administered as a single dose and/or in a physically discrete unit of a pharmaceutical composition. In many embodiments, a unit dose contains a predetermined quantity of an active agent, for instance a predetermined viral titer (the number of viruses, virions, or viral particles in a given volume). In some embodiments, a unit dose contains an entire single dose of the agent. In some embodiments, more than one unit dose is administered to achieve a total single dose. In some embodiments, administration of multiple unit doses is required, or expected to be required, in order to achieve an intended effect. A unit dose can be, for example, a volume of liquid (e.g., an acceptable carrier) containing a predetermined quantity of one or more therapeutic moieties, a predetermined amount of one or more therapeutic moieties in solid form, a sustained release formulation or drug delivery device containing a predetermined amount of one or more therapeutic moieties, etc. It will be appreciated that a unit dose can be present in a formulation that includes any of a variety of components in addition to the therapeutic moiety(s). For example, acceptable carriers (e.g., pharmaceutically acceptable carriers), diluents, stabilizers, buffers, preservatives, etc., can be included. It will be appreciated by those skilled in the art, in many embodiments, a total appropriate daily dosage of a particular therapeutic agent can include a portion, or a plurality, of unit doses, and can be decided, for example, by a medical practitioner within the scope of sound medical judgment. In some embodiments, the specific effective dose level for any particular subject or organism can depend upon a variety of factors including the disorder being treated and the severity of the disorder; activity of specific active compound employed; specific composition employed; age, body weight, general health, sex, and diet of the subject; time of administration, and rate of excretion of the specific active compound employed; duration of the treatment; drugs and/or additional therapies used in combination or coincidental with specific compound(s) employed, and like factors well known in the medical arts.

Vector: A “vector” is a nucleic acid molecule that is capable of transporting another nucleic acid molecule (including without limitation a nucleic acid molecule that is a fragment of the vector), such as a gene encoding a therapeutic gene.

### BRIEF DESCRIPTION OF THE DRAWINGS

FIG. 1 is an annotated alignment of protein sequences for CD33 proteins (amino-terminus through the transmembrane domain, but not including the cytoplasmic domain) from (in order from top to bottom) Macaca fascicularis (SEQ ID NO: 1), Homo sapiens (SEQ ID NO: 2), and Mus musculus (SEQ ID NO: 3). Full length sequences are shown in SEQ ID NOs: 170, 14, and 171, respectively.

FIGS. 2A-2B. FIG. 2A is a schematic drawing of antibody targeting of CD33. FIG. 2B is a depiction of the anti-CD33 antibody-drug conjugate gemtuzumab ozogamicin (GO).

FIGS. 3A-3B illustrate an exemplary base-editing strategy to reduce or inactivate CD33 expression. FIG. 3A illustrates generally how a cytidine base editor (CBE) functions to switch C with T. FIG. 3B illustrates an embodiment of using a CBE to inactivate the intron1 splicing donor site of CD33 (5′-to-3′ sequence in SEQ ID NO: 4; corresponding gRNA shown in SEQ ID NO: 194), and an embodiment of using a CBE to introduce a stop codon into exon 2 of CD33 (5′-to-3′ sequence in SEQ ID NO: 5; corresponding gRNA shown in SEQ ID NO: 195).

FIGS. 4A-4B. FIG. 4A illustrates elements used in the analysis of CBE modification of CD33, including electroporation to introduce CBE mRNA to target cells, and analysis of the resultant cells by CD33 surface-expression flow cytometry as well as editing efficiency using next generation sequencing (NGS). FIG. 4B is a graph showing percent of CD33 expression in human CD34+ HSPCs, measured using CD33 surface expression flow cytometry, through the indicated time course. Cells were edited with Cas9 only (solid circles), CBE at E2 (X), CBE at E1 (solid triangles), and CRISPR (solid squares).

FIGS. 5A-5C. Editing efficiency of CD33 CBE in human CD34+ HSPCs. FIG. 5A shows a schematic illustrating the results of CRISPR/Cas9-mediated E2 deletion in CD33 (top panel); and a graph of percent of CD33 expression in CD34+ HSPCs, measured using CD33 surface expression flow cytometry, in cells treated with Cas9 only (negative control) and the complete CRISPR system. FIG. 5B is a pair of graphs illustrating the specific base changes produced by CBE editing of E1 (SEQ ID NO: 193); the left panel shows results from cells treated with Cas9 only (negative control), which shows essentially no changes; and the right panel shows results from cells treated with the full CBE system to edit E1, which shows editing of up to about 30% at the targeted G residue, with lower (10-15% editing) at the two 5′ G positions. FIG. 5C is a pair of graphs illustrating the specific base changes produced by CBE editing of E2 (SEQ ID NO: 13); the left panel shows results from cells treated with Cas9 only (negative control), which shows essentially no changes; and the right panel shows results from cells treated with the full CBE system to edit E2, which shows editing of up to about 8% at the targeted G residue.

FIG. 6 illustrates a system used for examining engraftment of CBE-treated human CD34+ HSPCs in a mouse transplantation model. The top panel is a table showing the four treatment groups; the bottom panel illustrates the timing for injections and testing in the mice.

FIG. 7 is a pair of graphs showing results from the experimental system illustrated in FIG. 6, illustrating normal engraftment and differentiation of CBE-treated CD34+ HSPCs through the 18 weeks tested, including in the CD14+ sub-fraction of human CD45+ cells (bottom graph).

FIGS. 8A-8B illustrate that CBE editing and CD33 knockdown are persistent in vivo. FIG. 8A shows, in the first graph, the percent of CD33 expression in CD14+ cells treated with Cas9 only (solid circles), CBE at E2 (X's), CBE at E1 (solid triangles), and CRISPR (solid squares) over an 18 week time course after edited cells were introduced into mice. Side graphs illustrate the specific nucleotide edits found at 10 weeks in a specific mouse (#1874) that received cells edited at E2 using CBE, at 10 weeks in a specific mouse (#1904) that received cells edited at E1 using CBE, and the percent of CD33 E2 deletion persisting in the mice treated with CRISPR-edited cells at 18 weeks post treatment. FIG. 8B is a graph showing the percentage of CD33 expression in mice treated with the indicated edited cells, over 12 days post-infusion.

FIGS. 9A-9B are a pair of graphs, showing correlation between in vivo CD33 editing levels and protection from GO-induced cytotoxicity. Three mice per group were treated with GO (FIG. 9A), and a sharp decrease in the number of CD14+ monocytes was observed 1 wk post treatment, showing that the drug is active. Interestingly, the magnitude of the decrease was inversely correlated with editing efficiency. The sharper decrease was seen in the control group and a smaller effect was observed in the CRISPR group where editing efficiency was highest. The recovery in CD14+ cell number over time suggests that progenitor or stem cells are able to replenish the pool of monocytes and were thus not affected by treatment. FIG. 9B shows the parallel control experiment, without GO treatment.

FIGS. 10A-10B are a pair of graphs showing recovery of CD33 expression in HSCs after treatment, in the same experiment shown in FIGS. 9A-9B.

FIGS. 11A-11B are a pair of graphs showing that GO has no effect on CD33 negative cell lineages, in the same experiment shown in FIGS. 9A-9B.

FIGS. 12A and 12B are schematic drawings of the ABE8e (FIG. 12A; Addgene #138489; SEQ ID NO: 6) and the ABE8e-NG (FIG. 12B; Addgene #138491; SEQ ID NO: 7) plasmids. The development of these plasmids is described in Richter et al. (Nat. Biotech. 38(7):883-891, 2020).

FIGS. 13A-13B illustrate a system for targeting of two gamma globin (HBG) promoter target sites (−113 and −175) with ABE8e in nonhuman primate NHP CD34+ cells for the reactivation of fetal hemoglobin. FIG. 13A is a schematic of HBG target sites (5′-to-3′ sequences in SEQ ID NOs: 8 and 9). FIG. 13B is a pair of sequencing chromatograms showing editing efficiency measured by EditR analysis (Kluesner et al., CIRSPR J. 1(3):239-250, 2018; PMID: 31021262) (SEQ ID NOs: 10 and 11). Arrows show the position of edits; starred (★) boxes show frequencies of the targeted edits.

FIGS. 14A-14F show efficient CD33 knockdown with ABE8e. FIG. 14A shows targeting of CD33 splicing site (exon2 acceptor site) with ABE8e in NHP CD34+ cells (5′-to-3′ sequence in SEQ ID NO: 12). FIG. 14B is an illustration of conservation of the 3′ acceptor site; the AG that are boxed are universal, and therefore an excellent target for editing. The splicing acceptor site in exon 2 is inactivated, by editing the AG donor site to GG. FIG. 14C-14E show the editing efficiency of the CD33 target site, measured by EditR, in non-human primate (NHP) CD34+ cells mock treated (FIG. 14C), treated with ABE8e protein (FIG. 14D) or with ABE8e mRNA (FIG. 14E). Arrows show the position of edits; starred (★) boxes show frequencies. FIG. 14F shows flow cytometry analysis of CD33 surface expression in NHP CD34+ cells at six days post-treatment. (T234 rhesus CD34+ (6 days post EP)). SEQ ID NO: 13 is shown in each of FIGS. 14C, 14D, and 14E.

FIGS. 15A, 15B are a pair of graphs illustrating multiplex ABE8e HBG/CD33 editing in human fetal liver (FL) CD34+ cells. Cells were edited with ABE8e mRNA and single guide RNA (sgRNA) targeting the CD33 and HBG-175 sites. Editing efficiency was measured by next generation sequencing (NGS) at the CD33 (FIGS. 15A and 15B) or HBG-175 (FIGS. 15C-15E) sites. Graphs show editing frequency at each nucleotide within the target site. For CD33 in FIG. 15A, the C-to-T variation is due to a natural variant not induced by editing. FIG. 15F is a bar graphs showing there is minimal impact of multiplex editing on the capacity of human FLCD34+ cells to differentiate, using a colony forming analysis system. Cells were plated in a colony forming assay to evaluate the impact of editing on HSC multilineage differentiation. The graph shows the number of each type of differentiated cell (GEMM: granulocyte, erythroid macrophage, and megakaryocyte, GM: granulocyte-macrophage, G: granulocyte, M: macrophage, or BFU-E: burst-forming unit-erythrocyte) counted in colonies formed from plating edited 400 cells.

FIGS. 16A-16D is a series of graphs illustrating multiplex ABE8e HBG/CD33 editing in human mobilized peripheral blood (mPB) CD34+ cells. Cells were edited with ABE8e mRNA and single guide RNA (sgRNA) targeting each of the CD33 and HBG-175 sites. Editing efficiency was measured by EditR at the CD33 or HBG-175 sites. Arrows show the position of edits; starred (★) boxes show editing frequencies. SEQ ID NO: 13 is shown in each of FIGS. 16A and 16B; SEQ ID NO: 11 is shown in each of FIGS. 16C and 16D.

FIGS. 17A-17E illustrate ABE8e CD33 editing in NHP CD34+ cells. Cells were edited with two different concentrations (high and low) of ABE8e mRNA and single guide RNA (sgRNA) targeting CD33. FIG. 17A is three panels showing editing efficiency measured by EditR. Arrows show the position of edits, and starred (★) boxes show editing frequencies; SEQ ID NO: 13 is shown in all three sequencing chromatograms. FIG. 17B is a bar graph showing percentage of CD33 expression in the same edited cells, measured by flow cytometry analysis. FIG. 17C is a bar graphs showing there is minimal impact of CD8e editing using either high or low mRNA on the capacity of human FLCD34+ cells to differentiate, measured using a colony forming analysis system. Cells were plated in a colony forming assay to evaluate the impact of editing on HSC multilineage differentiation, as in FIG. 15B. FIG. 17D is a schematic drawing of mono- vs. bi-allelic CD33 editing; 5′-to-3′ sequence in SEQ ID NO: 12. FIG. 17E is a pair of pie graphs showing the percentage of mono- vs. bi-allelic CD33 editing frequencies (in treated NHP CD34+ cells) measured in single colonies (n=49) at either targeted adenine.

FIGS. 18A-18B illustrate multiplex ABE8e HBG/CD33 editing in NHP CD34+ cells and analysis of single- vs. double-edits at a single cell level. FIG. 18A is an outline of the experimental procedure. FIG. 18B is a pie graph showing the frequency of single- vs. double-edits in derived colonies (n=46) from treated CD34+ cells. Over half of the colonies show editing at both targets.

FIGS. 19A-19C show ABE8e CD33 editing in NHP HSPC subsets. NHP CD34+ cells (bottom panel, FIG. 19A) were treated with ABE8e mRNA or RNPs targeting CD33 and subsequently sorted for the different HSPC subpopulations: CD34+ (FIG. 19A, top panel), CD90+, CD90− and CD45RA+ (FIG. 19B). Validation of the purity of the sorting experiment is shown. CD33 editing efficiency in the different subpopulations from FIG. 19A-19B, measured by NGS, is shown in cells treated with ABE8e mRNA (FIG. 19C, top panel) or RNPs (FIG. 19C, bottom panel). SEQ ID NO: 13 is shown in both graphs of FIG. 19C.

FIGS. 20A, 20B illustrate engraftment of multiplex edited ABE8e HBG/CD33 FL CD34+ in immunodeficient mice. FIG. 20A is a pair of graphs showing longitudinal tracking of human cell engraftment based on human CD45+ flow cytometry staining from peripheral blood over 21 weeks (left) or from spleen and bone marrow of transplanted mice at the time of necropsy (right). FIG. 20B is a pair of graphs showing persistence of CD33 knockdown after engraftment. Longitudinal tracking of CD33 expression from peripheral blood over 21 weeks (left) or from spleen and bone marrow of transplanted mice at the time of necropsy (right); untreated cells are solid squares, and multiplex edited cells are solid circles. Edited cells show very little CD33 expression over time, compared to control.

FIG. 21 is a graph illustrating cytotoxicity of BEs vs CRISPR/Cas9 when delivered using a HDAd vector. Cytotoxicity of HDAds expressing BEs vs CRISPR/Cas9. Human CD34+ HSPCs were transduced with the CRISPR vector HDAd-HBG-CRISPR, the BE vector HDAd-ABE-sgHBG #2, or a control vector HDAd-GFP at a MOI 3000vp/cell. Twenty-four hours after transduction, the cells were used for in vivo engraftment studies. Cells were intravenously infused into irradiated NSG mice at 5×10⁵ cells/mouse (n=3 per treatment). Untransduced cells or HSPCs transduced with a GFP-expressing vector (HDAd-GFP) were used as controls. Engraftment reflected by % human CD45+ cells in PBMCs at the indicated weeks after infusion was measured by flow cytometry. Each dot represents one animal. *, p<0.05. ns, not significant. This graph shows that engraftment (a critical functional feature of HSC) of edited cells in sub-lethally irradiated NSG mice is not affected by transduction of human huCD45+ cells with a BE expressing adenoviral vector (HDAd-ABE-sgHBG #2), but is dramatically reduced after transduction of human CD34+ cells with a CRISPR/Cas9 expressing adenoviral vector (HDAd-HBG-CRISPR).

FIG. 22 is a general schematic of HDAd35 vector production; Features of representative Ad35 helper virus and vectors described herein. The five-point star indicates the following text: -combination (addition and reactivation) for SB100× and targeted; -multiple sgRNAs for CRISPR or BE; -miRNA (miR187/218) regulated expression of Cas9; and -auto-inactivation of Cas9.

FIG. 23. The left end of Ad5/35 helper virus genome (SEQ ID NO: 186). The sequences shaded in dark grey correspond to the native Ad5 sequence, i.e., the unshaded or light grey highlighted sequences were artificially introduced. The sequences highlighted in light grey are 2 copies of the (tandemly repeated) loxP sequences. In the presence of “cre recombinase” protein, the nucleotide sequence between the two loxP sequences are deleted (leaving behind one copy of loxP). Because the Ad5 sequence between the loxP sites is essential for packaging the adenoviral DNA into capsids (in the nucleus of the producer cell), this deletion results in the helper adenovirus genome DNA not to be packageable. Consequently, the efficiency of the deletion process has a direct influence of the level of packaged helper genomic DNA (the undesired helper virus “contamination”). In view of the above, in order to translate the same scheme to adenovirus serotypes other than Ad5, it is desirable to achieve the following: 1. Identify the sequences that are essential for packaging, so that they can be flanked by loxP sequence insertions and deleted in the presence of cre recombinase. Identification of these sequences is not straightforward if there is little similarity in sequences. 2. Determine where in the native DNA sequence the insertion of loxP sequence would have the least effect for the propagation and packaging of helper virus (in the absence of cre recombinase). 3. Determine the spacing between the loxP sequences to allow for efficient deletion of packaging sequences and keeping helper virus packaging to a minimum during the production of helper-dependent adenovirus (i.e., in a cre recombinase—expressing cell line such as the 116 cell line).

FIG. 24. Alignment of Ad5 and Ad35 packaging signals (SEQ ID NOs: 187 and 188). The alignment of the left end sequences of Ad5 with Ad35 help in identifying packaging signals. The motifs in the Ad5 sequence that are important for packaging (AI through AV) are in boxes (see FIG. 1B of Schmid et al., J Virol., 71(5):3375-4, 1997). The location of the loxP insertion sites are indicated by black arrows. It is seen that the insertions flank AI to AIV and disrupt AV. Please note that the additional packaging signal AVI and AVII, as indicated in Schmid et al., have been deleted in the Ad5 helper virus as part of the E1 deletion of this vector.

FIG. 25. Schematic of pAd35GLN-5E4. This is a first-generation (E1/E3-deleted) Ad35 vector derived from a vectorized Ad35 genome (Holden strain from the ATCC) using a recombineering technique (Zhang et al., Cell Rep. 19(8):1698-17-9, 2017). This vector plasmid was then used to insert loxP sites.

FIG. 26. Information on plasmid packaging signals. The packaging site (PS)1 LoxP insertion sites are after nucleotide 178 and 344. This should remove AI to AIV. The rest of the packaging signal including AVI and AVII (after 344) has been deleted (as part of the E1 deletion (345 to 3113)). The PS2 LoxP insertion sites are after nucleotide 178 and 481. Additionally, nucleotides 179 to 365 have been deleted, so AI through AV are not present. The remaining packaging motifs AVI and AVII are removable by cre recombinase during HDAd production. The E1 deletion is from 482 to 3113. The PS3 LoxP insertion sites are after nucleotide 154 and 481. Three engineered vectors could be rescued. The percentage of viral genomes with rearranged loxP sites was 50, 20, and 60% for PS1, PS2, and PS3, respectively. Rearrangements occur when the lox P sites critically affected viral replication and gene expression. Vectors with rearranged loxP sites can be packaged and will contaminate the HDAd prep. SEQ ID NOs: 180, 172, and 173 exemplify the vectors diagramed as PS1, PS2, and PS3, respectively.

FIG. 27. Next generation HDAd35 platform compared to current HDAd5/35 platform. Both vectors contain a CMV-GFP cassette. The Ad35 vector does not contain immunogenic Ad5 capsid protein. Shows comparable transduction efficiency of CD34+ cells in vitro. Bridging study shows comparable transduction efficiency of CD34+ cells in vitro. Human HSCs, peripheral CD34+ cells from G-CSF mobilized donors were transduced with HDAd35 (produced with Ad35 helper P-2) or a chimeric vector containing the Ad5 capsid with fiber from Ad35, at MOIs 500, 1000, 2000 vp/cell. The percentage of GFP-positive cells was measured 48 hours after adding the virus in three independent experiments. Notably, infection with HDAd35 triggered cytopathic effect at 48 hours due to helper virus contamination.

FIG. 28. The PS2 helper vector was remade to focus on monkey studies. The following are actions learned from: deletion of E1 region, a mutant packaging signal flanked by Loxp, mutant packaging sequence, deletion of E3 region (27435430540), replace with Ad5E4orf6, insertion of stutter DNA flanking copGFP cassette, and introduction of mutation in the knob to make Ad35K++.

FIG. 29. Mutated packaging signal sequence provided (SEQ ID NO: 181). Residues 1 through 137 are the Ad35 ITR (SEQ ID NO: 182). Text in bold are the SwaI sites, the Loxp site is italicized (SEQ ID NO: 184), and the mutated packaging signal is underlined (SEQ ID NO: 185).

FIGS. 30A, 30B. Schematic drawings of various helper vector and packaging signal variants. In embodiments, the E3 region (27388→30402) is deleted and the CMV-eGFP cassette is located within an E3 deletion, Ad35K++, and eGFP is used instead of copGFP. All four helper vectors containing the packaging signal variants shown in (FIG. 30A) could be rescued. loxP sites were rearranged as amplification could be more efficient. Additional packaging signal variants are exemplified in FIG. 30B.

FIG. 31 provide diagrams of additional helper-dependent adenoviral vectors (HDAds) expressing BE. The overall structure of HDAd-CBE/ABE vectors contains a 4.2 kb mgmt^{P140K}/GFP transposon flanked by two frt-IRs and an around 9 kb base editor cassette. The transposon allows for integrated expression when co-delivered with HDAd-SB expressing SB100× transposase and flippase (Flpe). The BE cassette was placed outside of the transposon for transient expression. The 1St version of HDAd-ABE vectors was not rescuable. The 2^{nd} version of HDAd-ABE vector design contains two codon-optimized TadAN repeats to reduce sequence repetitiveness (N denotes new; * denotes the catalytic repeat). A microRNA responsive element (miR) was embedded in the 3′ human p-globin UTR to minimize toxicity to producer cells by specifically downregulating ABE expression in 116 cells. bGHpA, bovine growth hormone polyadenylation sequence. T2A, a self-cleaving 2A peptide. PGK, human PGK promoter. U6, human U6 promoter. rAPOBEC1, cytidine deaminase enzyme. 32aa or 9aa, linker with 32 or 9 amino acids. SpCas9n, SpCas9 nickase. UGI, uracil glycosylase inhibitor. SV40pA, simian virus 40 polyadenylation signal. TadA, adenosine deaminase. ITR, inverted terminal repeat. ψ, packaging signal.

FIG. 32 Detection of intergenic deletion. The detection of intergenic 4.9 k deletion was described previously (Li et al., Blood, 131(26): 2915, 2018). Genomic DNA isolated from total bone marrow MNCs were used as template. A 9.9 kb genomic region spanning the two CRISPR cutting sites at HBG1 and HBG2 promoters was amplified by PCR. An extra 5.0 kb band in the product indicates the occurrence of the 4.9 k deletion. The percentage of deletion was calculated according to a standard curve formula which was generated by PCR using templates with defined ratios of the 4.9 kb deletion. Samples derived from mice in vivo transduced with a CRISPR vector targeting HBG1/2 promoter were used in comparison. Each lane represents one animal.

### DETAILED DESCRIPTION

The present disclosure provides, among other things, base editing for selective protection of therapeutic cells from an anti-CD33 agent. In certain embodiments, base editing reduces expression of CD33 by therapeutic cells as compared to a reference, e.g., such that contacting therapeutic cells with an anti-CD33 agent is less likely to eliminate the therapeutic cells. As disclosed herein, therapeutic cells can include any cells that express CD33 and/or are therapeutic at least in that they cause, elicit, or contribute to a desired pharmacological and/or physiological effect. In various embodiments, therapeutic cells are HSCs of a subject and the anti-CD33 agent is administered to the subject to treat cancer. In various embodiments, therapeutic cells are HSCs of a subject and the anti-CD33 agent is administered to positively select cells engineered for reduced CD33 expression as compared to a reference. In various embodiments, elimination of cells refers to causing the death, cessation of growth, cessation of proliferation, and/or cessation of one or more biological functions of a cell, e.g., as understood by those of skill in the art to result from contact of a cell with a particular agent such as an anti-CD33 agent.

### CD33

CD33 expression is characteristic of certain therapeutically relevant cell types, including without limitation HSCs. In various embodiments, CD33 expression is characteristic of one or more cells and/or cell types that would be beneficial to positively select and/or selectively protect. To provide a first non-limiting example, in some embodiments, cancer cells express CD33 such that the cancer can be treated by an anti-CD33 agent, but certain beneficial cells also express CD33 such that it would be advantageous to selectively protect the beneficial cells from the anti-CD33 agent. To provide a second non-limiting example, in some embodiments, CD33-expressing cells are genetically engineered to include a therapeutic modification and a modification that decreases CD33 expression, such that an anti-CD33 agent can positively select for engineered cells.

CD33-targeting agents can bind different forms and/or epitopes of CD33. A typical human CD33 protein can have an amino acid sequence according to SEQ ID NO: 14. Additional full length sequences of representative CD33 proteins are shown in SEQ ID NO: 169 (Macaca mulatta), SEQ ID NO: 170 (Macaca fascicularis), and SEQ ID NO: 171 (Mus musculus). An exemplary human genome sequence encoding CD33 can be a sequence according to SEQ ID NO: 15, from which various transcripts can be expressed including without limitation NM_001772 (full length), NM_001082618, and NM_001177608 (see, e.g., Laszlo, Oncotarget 7(28):43281-43294, 2016).

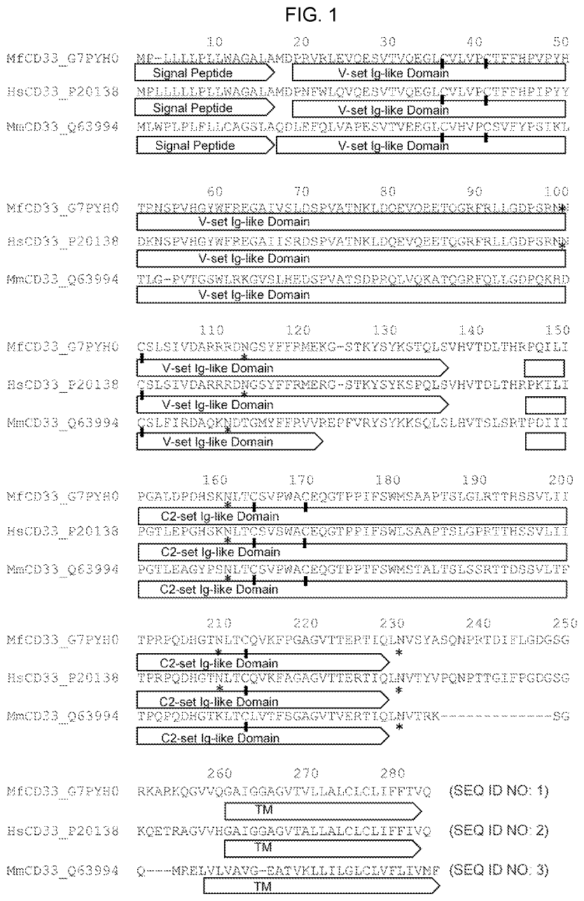

CD33 can include a number of domains including a signal peptide domain, a V-set Ig-like domain (mediates sialic acid binding), a C2-set Ig-like domain, a transmembrane domain, and a cytoplasmic tail. In various exemplary embodiments, a signal peptide domain corresponds to amino acids 1-16 of SEQ ID NO: 14, a V-set Ig-like domain (mediates sialic acid binding) corresponds to amino acids 17 or 19-135 of SEQ ID NO: 1, a C2-set Ig-like domain corresponds to amino acids 145-228 of SEQ ID NO: 14, a transmembrane domain correspond to amino acids 260-282 of SEQ ID NO: 14, and a cytoplasmic tail (includes conserved tyrosine-based inhibitory signaling motifs) corresponds to amino acids 283-364 of SEQ ID NO: 14. For example, full length CD33 (CD33FL) (SEQ ID NO: 14) is a transmembrane glycoprotein that is characterized by an amino-terminal, membrane-distant V-set immunoglobulin (Ig)-like domain and a membrane-proximal C2-set Ig-like domain in its extracellular portion. In addition to CD33FL, a splice variant that misses exon 2 (CD33ΔE2) (SEQ ID NO: 17) has also been identified. Various studies have identified an AML-expressed CD33 splice variant missing exon 2 (CD33ΔE2) and consequently lacking the immune-dominant membrane-distal V-set domain as well as CD33 splice variants lacking all or a portion of other domains such as the intracellular domain. Thus, CD33 can refer to, among other things, any native, mature CD33 which results from processing of a CD33 precursor protein in a cell (FIG. 1; SEQ ID NOs: 14 and 169-171).

CD33 is typically primarily displayed on maturing and mature cells of the myeloid lineage, including multipotent myeloid precursors. CD33 is a protein that is expressed on normal hematopoietic cells as they mature. Thus, in various embodiments, therapeutic cells that can be administered as a treatment for immune deficiencies or other blood-related disorders can be therapeutic cells that express or begin to express CD33. CD33 is not typically found on pluripotent hematopoietic stem cells or non-blood cells.

CD33 is widely expressed on neoplastic cells in patients with a variety of hematologic disorders, such as myelodysplastic syndrome (MDS) or acute myeloid leukemia (AML). Accordingly, CD33 represents a cellular marker for both administered therapeutic cells and unwanted non-treated, cancerous, and/or malignant cells within a patient. Consistent with its role as a myeloid differentiation antigen, CD33 is widely expressed on malignant cells in patients with myeloid neoplasms, particularly acute myeloid leukemia (AML), where it is displayed on at least a subset of the leukemia blasts in almost all cases and possibly leukemia stem cells in some. Because of this expression pattern, there has been great interest in developing therapeutic antibodies directed at CD33. While unconjugated monoclonal CD33 antibodies proved ineffective in patients with AML, several randomized trials with the CD33 antibody-drug conjugate (ADC) gemtuzumab ozogamicin (GO) (MYLOTARG®, Pfizer, New York, N.Y.) have demonstrated improved survival in some AML patients. This establishes the value of antibodies in this disease and validates CD33 as the first and (so far) only therapeutic target for AML immunotherapy. This benefit of GO in randomized trials led to regulatory re-approval by the U.S. Food & Drug Administration (FDA) in 2017 for the treatment of newly-diagnosed as well as relapsed or refractory CD33-expressing AML. In 2018, GO was also approved by the European Medicines Agency (EMA) in Europe for the treatment of patients 5 years in combination with intensive chemotherapy for the treatment of newly-diagnosed de novo AML.

At least in part because CD33 can be a target for agents to kill diseased and/or unwanted cells, there has been great interest in developing therapeutic antibodies directed at CD33. However, because CD33 is also expressed on normal immune cells and other non-malignant cells, treatments that target it have created what are referred to as significant “on-target, but off-leukemia” or “on-target, off-tumor” effects.” The expression of CD33 on maturing and mature cells of the myeloid lineage leads to significant on-target, off-leukemia effects of CD33-targeted immunotherapy. Such effects include suppression of the blood and immune system in the forms of severe thrombocytopenia, neutropenia, and monocytopenia in patients. For example, the CD33 ADC GO when given alone causes almost universal severe thrombocytopenia and neutropenia (thus, for example, with GO monotherapy given at standard dose, grade 3/4 toxicities include invariable myelosuppression), and when combined with conventional chemotherapy GO has resulted in prolongation of cytopenias and increased non-relapse related mortality, in part due to increased frequency of fatal infections, in some clinical trials. Some non-randomized studies similarly reported substantially increased hematologic toxicities with the use of GO together with conventional chemotherapeutics, indicating a narrow therapeutic window. Several CD33-targeting therapeutics, including newer-generation ADCs (SGN-CD33A, IMGN779), bispecific antibodies (AMG330, AMG673, AMV-564), and CAR-modified T-cells have entered clinical testing and are more potent than GO. Among these recently developed investigational agents, most advanced in development was SGN-CD33A, with clinical data from early-phase clinical trials indicating not only anti-leukemia efficacy but also the potential to cause prolonged cytopenias and life-threatening sequelae (e.g., bleeding, infection). The latter problem is perhaps best exemplified by the premature termination of the CASCADE trial (phase 3 trial testing SGN-CD33A in addition to DNA methyltransferase inhibitor) because of an increase in deaths including fatal infections. In fact, partly because of these results, SGN-CD33A is no longer currently pursued as a clinical therapeutic. For various other newer-generation anti-CD33 therapeutics, highly effective elimination of CD33-positive cells is expected to cause very prolonged cytopenias and increase risks of infection and bleeding with such potent CD33-targeted immunotherapies.

The experience with GO and SGN-CD33A suggests that clinically-relevant toxicity of CD33-targeted immunotherapy could be minimized in the presence of normal hematopoietic cells that do not display, or have reduced expression of the CD33 antigen.

To address the described “on-target, off-tumor” effects, strategies have been developed to protect beneficial therapeutic hematopoietic cells from anti-CD33 therapies while leaving residual diseased cells susceptible to anti-CD33 treatments. Certain of these strategies achieve this benefit by genetically modifying HSC to have reduced or eliminated expression of CD33, thus protecting them from anti-CD33 based therapies. In this manner, genetically modified therapeutic cells will not be harmed by concurrent or subsequent anti-CD33 therapies a patient may receive. However, in various embodiments, pre-existing CD33-expressing cells in the patient and/or administered cells that lack the genetic modification will not be protected, resulting in positive selection for the therapeutic cells over other cells.

One approach to reducing CD33 expression that has been explored includes utilizing CRISPR/Cas9 nuclease-based gene editing of CD34+ hematopoietic stem and progenitor cells (HSPCs) to reduce CD33 expression. This strategy successfully conferred protection from CD33-directed drugs. While promising, there were also drawbacks associated with this CRISPR/Cas9-based strategy. For example, the CRISPR/Cas9 nuclease also suffers from off-target activity due to cleavage of a nearby CD33 homolog pseudogene and from activation of endogenous TP53-mediated DNA damage responses.

Base Editing Systems for Inactivation of CD33 in Therapeutic and/or CD33-Expressing Cells

The present disclosure includes methods and compositions that relate to base editing of nucleic acid sequences to reduce CD33 expression in therapeutic cells as compared to a reference, e.g., by base editing of nucleic acid sequences that encode and/or contribute to expression of CD33. A base editing system can include a base editing enzyme and/or at least one gRNA as components thereof. As disclosed herein, an anti-CD33 agent may be administered to a subject or system, e.g., to selectively target and/or eliminate cells such as cancer cells that express CD33 or to positively select for engineered cells. In embodiments in which an anti-CD33 agent selectively targets and/or eliminates cells such as cancer cells that express CD33, the present disclosure contemplates that it may be desirable to protect during anti-CD33 therapy certain cells of therapeutic value that typically express CD33, e.g., HSCs. Accordingly, administration to the subject or system of agent(s) that inactivate CD33 in cells of therapeutic value (e.g., HSCs), e.g., without inactivating CD33 in target cells such as cancer cells, can improve therapy and/or provide a therapeutic benefit. In embodiments in which cells that typically express CD33 are engineered by an agent that introduces a therapeutic genetic modification, the agent can be engineered such that it can also inactivate CD33 expression, such that CD33 inactivation becomes a biomarker of the therapeutic genetic modification and allows positive selection of therapeutically genetically modified cells upon administration to the subject or system of an anti-CD33 agent. As provided herein, base editing systems and/or techniques provide at least certain compositions and methods for CD33 inactivation.

As disclosed herein, CD33 inactivation includes modification of one or more genomic sequences that encode CD33, contribute to expression of CD33, or are operably linked to sequences that encode or contribute to expression of CD33, where the modification of the one or more genomic sequences reduces expression of CD33 (and/or expression of a form of CD33 capable of being bound by anti-CD33 agents, e.g., one or more particular anti-CD33 agents, e.g., one or more particular anti-CD33 agents of the present disclosure) as compared to a reference. CD33 inactivation as disclosed herein further includes reduction of expression of CD33 (and/or expression of a form of CD33 capable of being bound by anti-CD33 agents, e.g., one or more particular anti-CD33 agents, e.g., one or more particular anti-CD33 agents of the present disclosure) as compared to a reference. Exemplary genomic CD33 sequences that can be modified to inactivate CD33 can include CD33 exons, CD33 introns, CD33 promoters, CD33 untranslated regions (UTRs), and the like. Reduced expression of CD33 can refer to any decrease in rate of production or amount of CD33 transcripts and/or CD33 polypeptides by a cell or population of cells, e.g. a population of cells of a particular type. Reduced expression can be determined by comparison to a reference, where the reference can be any of, without limitation, a sample or measurement representative of the same cell or population of cells prior to CD33 inactivation, a sample or measurement representative of a comparable cell or population of cells not subject to CD33 inactivation, or a standard, reference, or threshold value. In various embodiments, those of skill in the art will appreciate that a reference sample or measurement may be from a cell or population of cells under the same, similar, or comparable conditions. In some embodiments, a reference is a sample or measurement representative of a same, similar, or comparable cell type from the same individual prior to CD33 inactivation or from a different individual or group of individuals absent or prior to CD33 inactivation, In some embodiments a reference is a comparable cell or population of cells maintained in vitro, such as a laboratory strain. In some embodiments a reference is a value designated, known, and/or accepted as a normative or threshold value.

Base editing refers to the selective modification of a nucleic acid sequence by converting a base or base pair within genomic DNA or cellular RNA to a different base or base pair (Rees & Liu, Nature Reviews Genetics, 19:770-788, 2018). There are two general classes of DNA base editors: (i) cytosine base editors (CBEs) that convert guanine-cytosine base pairs into thymine-adenine base pairs, and (ii) adenine base editors (ABEs) that convert adenine-thymine base pairs to guanine cytosine base pairs. Broadly, a base editing system can include a base editing enzyme and at least one gRNA. DNA base editors can include a catalytically disabled nuclease fused to a nucleobase deaminase enzyme and, in some cases, a DNA glycosylase inhibitor. RNA base editors achieve analogous changes using components that base modify RNA. Components of most base-editing systems include (1) a targeted DNA binding polypeptide, (2) a nucleobase deaminase enzyme polypeptide and (3) a DNA glycosylase inhibitor polypeptide. In particular embodiments, a deaminase domain (cytidine and/or adenine) is fused to the N-terminus of the catalytically disabled nuclease. As indicated, most base-editing enzymes and/or systems include a DNA glycosylase inhibitor that serves to override natural DNA repair mechanisms that might otherwise repair the intended base editing. Presence of uracil glycosylase inhibitor suppresses natural DNA repair mechanisms following the base edit. For additional examples regarding such base editing systems, see WO 2018/165629A1. In particular embodiments, the DNA glycosylase inhibitor includes a uracil glycosylase inhibitor, such as the uracil DNA glycosylase inhibitor protein (UGI) described in Wang et al. (Gene 99, 31-37, 1991).

In particular embodiments, the targeted DNA binding protein can be a catalytically disabled nuclease. In particular embodiments, a targeted DNA binding protein with nickase activity is selected. Examples of such DNA binding proteins include nuclease-inactive Cas9 proteins. In particular embodiments, a Cas9 domain with high fidelity is selected wherein the Cas9 domain displays decreased electrostatic interactions between the Cas9 domain and a sugar-phosphate backbone of a DNA, as compared to a wild-type Cas9 domain. In some embodiments, a Cas9 domain (e.g., a wild type Cas9 domain) includes one or more mutations that decrease the association between the Cas9 domain and a sugar-phosphate backbone of a DNA. Cas9 domains with high fidelity are known to those skilled in the art. For example, Cas9 domains with high fidelity have been described in Kleinstiver et al. (Nature 529, 490-495, 2016) and Slaymaker et al. (Science 351, 84-88, 2015).

Particular embodiments utilize nuclease-inactive Cas9 (dCas9), a mutant of Cas9 containing D10A and H840A mutations allowing nickase activity, as the catalytically disabled nuclease. Additional embodiments utilize a Cas9 with the D10A mutation. However, any nuclease of the CRISPR system (many of which are described above) can be disabled and used within a base editing system. In particular embodiments, a Cas9 domain with high fidelity is selected wherein the Cas9 domain displays decreased electrostatic interactions between the Cas9 domain and a sugar-phosphate backbone of a DNA, as compared to a wild-type Cas9 domain. In some embodiments, a Cas9 domain (e.g., a wild type Cas9 domain) includes one or more mutations that decrease the association between the Cas9 domain and a sugar-phosphate backbone of a DNA. Cas9 domains with high fidelity are known to those skilled in the art. For example, Cas9 domains with high fidelity have been described in Kleinstiver et al. (Nature 529, 490-495, 2016) and Slaymaker et al. (Science 351, 84-88, 2015).

Beyond Cas9, any nuclease of the CRISPR system can be disabled and used within a base editing system. Additional exemplary Cas nucleases include CasI, CasIB, Cas2, Cas3, Cas4, Cas5, Cas6, Cas7, Cas8, Cas9 (also known as CsnI and CsxI2), CasIO, CpfI, C2c3, C2c2 and C2clCsyI, Csy2, Csy3, Cse1, Cse2, CscI, Csc2, Csa5, Csn2, Csm2, Csm3, Csm4, Csm5, Csm6, CmrI, Cmr3, Cmr4, Cmr5, Cmr6, CpfI, Csbl, Csb2, Csb3, CsxI7, CsxI4, CsxIO, CsxI6, CsaX, Csx3, CsxI, CsxI5, Csf1, Csf2, Csf3, and Csf4.

Nucleases from other gene-editing systems may also be used. For example, base-editing systems can utilize zinc finger nucleases (ZFNs) (Urnov et al., Nat Rev Genet. 2010; 11(9):636-46) and transcription activator like effector nucleases (TALENs) (Joung et al., Nat Rev Mol Cell Biol. 14(1):49-55, 2013). For additional information regarding DNA-binding nucleases, see US2018/0312825A1.

In particular embodiments, components from the CRISPR system are combined with other enzymes or biologically active fragments thereof to directly install, cause, or generate mutations such as point mutations in nucleic acids, e.g., into DNA or RNA, e.g., without making, causing, or generating one or more double-stranded breaks in the mutated nucleic acid. Certain such combinations of components are known as base editors.

Components of base editors can be fused directly (e.g., by direct covalent bond) or via linkers. For example, the catalytically disabled nuclease can be fused via a linker to the deaminase enzyme and/or a glycosylase inhibitor. Multiple glycosylase inhibitors can also be fused via linkers. As will be understood by one of ordinary skill in the art, linkers can be used to link any peptides or portions thereof.

Exemplary linkers include polymeric linkers (e.g., polyethylene, polyethylene glycol, polyamide, polyester); amino acid linkers; carbon-nitrogen bond amide linkers; cyclic or acyclic, substituted or unsubstituted, branched or unbranched aliphatic or heteroaliphatic linkers; monomeric, dimeric, or polymeric aminoalkanoic acid linkers; aminoalkanoic acid (e.g., glycine, ethanoic acid, alanine, beta-alanine, 3-aminopropanoic acid, 4-aminobutanoic acid, 5-pentanoic acid) linkers; monomeric, dimeric, or polymeric aminohexanoic acid (Ahx) linkers; carbocyclic moiety (e.g., cyclopentane, cyclohexane) linkers; aryl or heteroaryl moiety linkers; and phenyl ring linkers.

Linkers can also include functionalized moieties to facilitate attachment of a nucleophile (e.g., thiol, amino) from a peptide to the linker. Any electrophile may be used as part of the linker. Exemplary electrophiles include activated esters, activated amides, Michael acceptors, alkyl halides, aryl halides, acyl halides, and isothiocyanates.

In particular embodiments, linkers range from 4-100 amino acids in length. In particular embodiments, linkers are 4 amino acids, 9 amino acids, 14 amino acids, 16 amino acids, 32 amino acids, or 100 amino acids.

Base editors can directly convert one base or base pair into another, enabling the efficient installation of point mutations in non-dividing cells without generating excess undesired editing by-products, such as insertions and deletions (indels). For example, base editors can generate less than 10%, 9%, 8%, 7%, 6%, 5.5%, 5%, 4.5%, 4%, 3.5%, 3%, 2.5%, 2%, 1.5%, 1%, 0.5%, or 0.1% indels.

DNA base editors can insert such point mutations in non-dividing cells without generating double-strand breaks. Due to the lack of double-strand breaks, base editors do not result in excess undesired editing by-products, such as insertions and deletions (indels). For example, base editors can generate less than 10%, 9%, 8%, 7%, 6%, 5.5%, 5%, 4.5%, 4%, 3.5%, 3%, 2.5%, 2%, 1.5%, 1%, 0.5%, or 0.1% indels as compared to technologies that do rely on double-strand breaks.

In particular embodiments, the nucleobase deaminase enzyme is a cytidine deaminase domain or an adenine deaminase domain.

Certain particular embodiments utilize a cytidine deaminase domain as the nucleobase deaminase enzyme. Further, particular embodiments utilize a uracil glycosylase inhibitor (UGI) as a glycosylase inhibitor. For example, in particular embodiments, dCas9 or a Cas9 nickase can be fused to a cytidine deaminase domain. The dCas9 or a Cas9 nickase fused to the cytidine deaminase domain can be fused to one or more UGI domains. Base editors with more than one UGI domain can generate less indels and more efficiently deaminates target nucleic acids.

Particular embodiments can include a catalytically disabled CRISPR nuclease, such as a nuclease-inactive CRISPR-associated protein 9 (Cas9 (dCas9)) fused to a cytidine deaminase domain and a uracil glycosylase inhibitor. Particular embodiments can utilize a dCas9 or a Cas9 nickase fused to the cytidine deaminase domain can be fused to one or more glycosylase inhibitors, such as a UGI protein domain. Base editors with more than one UGI domain can generate less indels and more efficiently deaminate target nucleic acids. In certain embodiments, the base-editing system binds a specific nucleic acid sequence via the CRISPR nuclease domain, deaminates a cytosine within the nucleic acid sequence to a uridine. In particular embodiments, a deaminase domain (cytidine and/or adenine) is fused to the N-terminus of the catalytically disabled nuclease. A cytidine deaminase domain fused to the N-terminus of Cas9 can have improved base-editing efficiency when compared to other configurations. In these embodiments, a glycosylase inhibitor (e.g., UGI domain) can be fused to the C-terminus of the catalytically disabled nuclease. When multiple glycosylase inhibitors are used, each can be fused to the C-terminus of the catalytically disabled nuclease.

In particular embodiments, CBE utilizing a cytidine deaminase domain convert guanine-cytosine base pairs into thymine-adenine base pairs by deaminating the exocylic amine of the cytosine to generate uracil. Examples of cytosine deaminase enzymes include APOBEC1, APOBEC3A, APOBEC3G, CDA1, and AID. APOBEC1 particularly accepts single stranded (ss)DNA as a substrate but is incapable of acting on double stranded (ds)DNA. For CBEs, CRISPR-based editors can be produced by linking a cytosine deaminase with a Cas nickase, e.g., Cas9 nickase (nCas9). To provide one example, nCas9 can create a nick in target DNA by cutting a single strand, reducing the likelihood of detrimental indel formation as compared to methods that require a double-stranded break. After binding with DNA, the CBE deaminates a target cytosine (C) into a uracil (U) base. Later the resultant U-G pair is either repaired by cellular mismatch repair machinery making an original C-G pair converted to T-A or reverted to the original C-G by base excision repair mediated by uracil glycosylase. In various embodiments, expression of uracil glycosylase inhibitor (UGI), e.g., a UGI present in a payload, reduces the occurrence of the second outcome and increases the generation of T-A base pair formation.

Numerous base-editing (BE) systems formed by linking targeted DNA binding proteins with cytidine deaminase enzymes and DNA glycosylase inhibitors (e.g., UGI) have been described. These complexes include for example, BE1 ([APOBEC1-16 amino acid (aa) linker-Sp dCas9 (D10A, H840A)] Komer et al., Nature, 533, 420-424, (2016)), BE2 ([APOBEC1-16aa linker-Sp dCas9 (D10A, H840A)-4aa linker-UGI] Komer et al., 2016 supra), BE3 ([APOBEC1-16aa linker-Sp nCas9 (D10A)-4aa linker-UGI] Komer et al., supra), HF-BE3 ([APOBEC1-16aa linker-HF nCas9 (D10A)-4aa linker-UGI] Rees et al., Nat. Commun. 8, 15790, (2017)), BE4, BE4max ([APOBEC1-32aa linker-Sp nCas9 (D10A)-9aa linker-UGI-9aa linker-UGI] Koblan et al., Nat. Biotechnol 10.1038/nbt.4172 (2018); Komer et al., Sci. Adv., 3, eaao4774, (2017)), BE4-GAM ([Gam-16aa linker-APOBEC1-32aa linker-Sp nCas9 (D10A)-9aa linker-UGI-9aa linker-UGI] Komer et al., 2017 supra), YE1-BE3 ([APOBEC1 (W90Y, R126E)-16aa linker-Sp nCas9 (D10A)-4aa linker-UGI] Kim et al., Nat. Biotechnol. 35, 475-480 (2017)), EE-BE3 ([APOBEC1 (R126E, R132E)-16aa linker-Sp nCas9 (D10A)-4aa linker-UGI] Kim et al., 2017 supra), YE2-BE3 ([APOBEC1 (W90Y, R132E)-16aa linker-Sp nCas9 (D10A)-4aa linker-UGI]Kim et al., 2017 supra), YEE-BE3 ([APOBEC1 (W90Y, R126E, R132E)-16aa linker-Sp nCas9 (D10A)-4aa linker-UGI] Kim et al., 2017 supra), VQR-BE3 ([APOBEC1-16aa linker-Sp VQR nCas9 (D10A)-4aa linker-UGI] Kim et al., 2017 supra), VRER-BE3 ([APOBEC1-16aa linker-Sp VRER nCas9 (D10A)-4aa linker-UGI] Kim et al., Nat. Biotechnol. 35, 475-480, (2017)), Sa-BE3 ([APOBEC1-16aa linker-Sa nCas9 (D10A)-4aa linker-UGI] Kim et al., 2017 supra), SA-BE4 ([APOBEC1-32aa linker-Sa nCas9 (D10A)-9aa linker-UGI-9aa linker-UGI] Komer et al., 2017 supra), SaBE4-Gam ([Gam-16aa linker-APOBEC1-32aa linker-Sa nCas9 (D10A)-9aa linker-UGI-9aa linker-UGI] Komer et al., 2017 supra), SaKKH-BE3 ([APOBEC1-16aa linker-Sa KKH nCas9 (D10A)-4aa linker-UGI] Kim et al., 2017 supra), Cas12a-BE ([APOBEC1-16aa linker-dCas12a-14aa linker-UGI], Li et al., Nat. Biotechnol. 36, 324-327 (2018)), Target-AID ([Sp nCas9 (D10A)-100aa linker-CDA1-9aa linker-UGI] Nishida et al., Science, 353, 10.1126/science.aaf8729 (2016)), Target-AID-NG ([Sp nCas9 (D10A)-NG-100aa linker-CDA1-9aa linker-UGI] Nishimasu et al., Science 2018 Sep. 21; 361(6408): 1259-1262), xBE3 ([APOBEC1-16aa linker-xCas9(D10A)-4aa linker-UGI] Hu et al., Nature, 556, 57-63 (2018)), eA3A-BE3 ([APOBEC3A (N37G)-16aa linker-Sp nCas9(D10A)-4aa linker-UGI] Gerkhe et al., Nat. Biotechnol., 10.1038/nbt.4199 (2018)), A3A-BE3 ([hAPOBEC3A-16aa linker-Sp nCas9(D10A)-4aa linker-UGI] Wang et al., Nat. Biotechnol. 10.1038/nbt.4198 (2018)), and BE-PLUS ([10× GCN4-Sp nCas9(D10A)/ScFv-rAPOBEC1-UGI] Jiang et al., Cell. Res, 10.1038/s41422-018-0052-4 (2018)). For additional examples of base editing agents and/or systems, including adenine deaminase base editors such as TadA*-dCas9, TadA-TadA*-Cas9, ABE7.9, ABE 6,3, and ABE7.10, see Rees & Liu Nat. Rev Genet. 2018 December; 19(12): 770-788.

Particular embodiments of the systems and methods disclosed herein utilize pCMV_BE4max (Addgene plasmid #112093; RRID:Addgene112093 and as described in Koblan et al., Nat. Biotechnol. 2018 May 29. pii: nbt.4172. doi: 10.1038/nbt.4172. 10.1038/nbt.4172). BE4max and AncBE4max are examples of cytosine base editors.

Particular embodiments of the systems and methods disclosed herein utilize base editors that are engineered to edit more than one type of nucleotide, such as both adenine and cytosine. Examples of such dual base editors are described in: Zhao et al. (“Glycosylase base editors enable C-to-A and C-to-G base changes.” Nat Biotechnol. 2020 doi: 10.1038/s41587-020-0592-2. PMID: 32690970), Kim et al. (“Adenine base editors catalyze cytosine conversions in human cells.” Nat Biotechnol. 37(10):1145-1148, 2019), Zhang et al. (“Dual base editor catalyzes both cytosine and adenine base conversions in human cells.” Nat Biotechnol. 38(7):856-860, 2020), and Grunewald et al. (“A dual-deaminase CRISPR base editor enables concurrent adenine and cytosine editing.” Nat Biotechnol. 38(7):861-864, 2020).

Certain particular embodiments utilize an adenine deaminase domain as the nucleobase deaminase enzyme. For ABEs, exemplary adenosine deaminases that can act on DNA for adenine base editing include a mutant TadA adenosine deaminases (TadA*) that accepts DNA as its substrate. E. coli TadA typically acts as a homodimer to deaminate adenosine in transfer RNA (tRNA). TadA* deaminase catalyzes the conversion of a target ‘A’ to ‘I’ (inosine), which is treated as ‘G’ by cellular polymerases. Subsequently, an original genomic A-T base pair can be converted to a G-C pair. As the cellular inosine excision repair is not as active as uracil excision, ABE does not require any additional inhibitor protein like UGI in CBE. In some embodiments, a typical ABE can include three components including a wild-type E. coli tRNA-specific adenosine deaminase (TadA) monomer, which can play a structural role during base editing, a TadA* mutant TadA monomer that catalyzes deoxyadenosine deamination, and a Cas nickase such as Cas9(D10A). In certain embodiments, there is a linker positioned between TadA and TadA*, and in certain embodiments there is a linker positioned between TadA* and the Cas nickase. In various embodiments, one or both linkers includes at least 6 amino acids, e.g., at least 5, 6, 7, 8, 9, 10, 15, 20, 25, 30, 35, 40, 45, or 50 amino acids (e.g., having a lower bound of 5, 6, 7, 8, 9, 10, or 15, amino acids and an upper bound of 20, 25, 30, 35, 40, 45, or 50 amino acids). In various embodiments, one or both linkers include 32 amino acids. In some embodiments, one or both linkers have a sequence according to (SGGS)2-XTEN-(SGGS)2 (SEQ ID NO: 18), or a sequence otherwise known to those of skill in the art. In this context, XTEN is a peptide linker with the sequence SGSETPGTSESATPES (SEQ ID NO. 183). See WO 2019/079374.

For additional information regarding base editors, see US 2018/0312825A1, WO 2018/165629A, Urnov et al., Nat Rev Genet. 11 (9):636-46, 2010; Joung et al., Nat Rev Mol Cell Biol. 14(1):49-55, 2013; Charpentier et al., Nature.; 495(7439):50-1, 2013; Seo & Kim, Nature Medicine, 24. 1493-1495. 2018, and Rees & Liu, Nature Reviews Genetics, 19, 770-78, 2018, each of which is incorporated herein by reference in its entirety and with specific respect to base editors. Certain base editor constructs that can be used in various embodiments of the present disclosure are described in Zafra et al., Nat Biotech, 36(9):888-893, 2018, and Koblan et al., Nat Biotech 36(9):843-846, 2018, each of which is incorporated herein by reference in its entirety and with specific respect to base editor constructs.

The present disclosure includes that ABEs and CBEs may be used in methods and compositions of the present disclosure to reduce CD33 expression in therapeutic cells as compared to a reference, e.g., by base editing of nucleic acid sequences that encode and/or contribute to expression of CD33. As will be clear from the present disclosure ABEs and CBEs are used in conjunction to gRNAs that mediate base editing activity. As those of skill in the art will appreciate from the present disclosure, any of a wide variety of therapeutic cell sequences can be targeted for modification by an ABE and/or CBE of the present disclosure to reduce expression of CD33.

In various embodiments, a base editor of the present disclosure introduces (e.g., in the presence of a gRNA that directs the base editing activity of the base editor) a nucleic acid sequence variation in a CD33-encoding sequence of a therapeutic cell that causes reduced expression of CD33 as compared to a reference. In various embodiments, a base editor of the present disclosure introduces a nucleic acid sequence variation in a CD33-encoding sequence of a therapeutic cell that causes reduced expression of full length CD33 as compared to a reference, e.g., reduced expression of CD33 polypeptides bound or capable of being bound by an anti-CD33 agent (e.g., one or more anti-CD33 agents of the present disclosure). In various embodiments, a base editor of the present disclosure introduces a nucleic acid sequence variation in a CD33-encoding sequence of a therapeutic cell that causes expression of a truncated CD33 polypeptide. In various embodiments, a base editor of the present disclosure introduces a nucleic acid sequence variation in a CD33-encoding sequence of a therapeutic cell that modifies or deletes the signal peptide domain of CD33. In various embodiments, a base editor of the present disclosure introduces a nucleic acid sequence variation in a CD33-encoding sequence of a therapeutic cell that modifies or deletes the V-set Ig-like domain of CD33. In various embodiments, a base editor of the present disclosure introduces a nucleic acid sequence variation in a CD33-encoding sequence of a therapeutic cell that modifies or deletes the C2-set Ig-like domain of CD33. In various embodiments, a base editor of the present disclosure introduces a nucleic acid sequence variation in a CD33-encoding sequence of a therapeutic cell that modifies or deletes the transmembrane domain of CD33. In various embodiments, a base editor of the present disclosure introduces a nucleic acid sequence variation in a CD33-encoding sequence of a therapeutic cell that modifies or deletes the cytoplasmic tail domain of CD33.

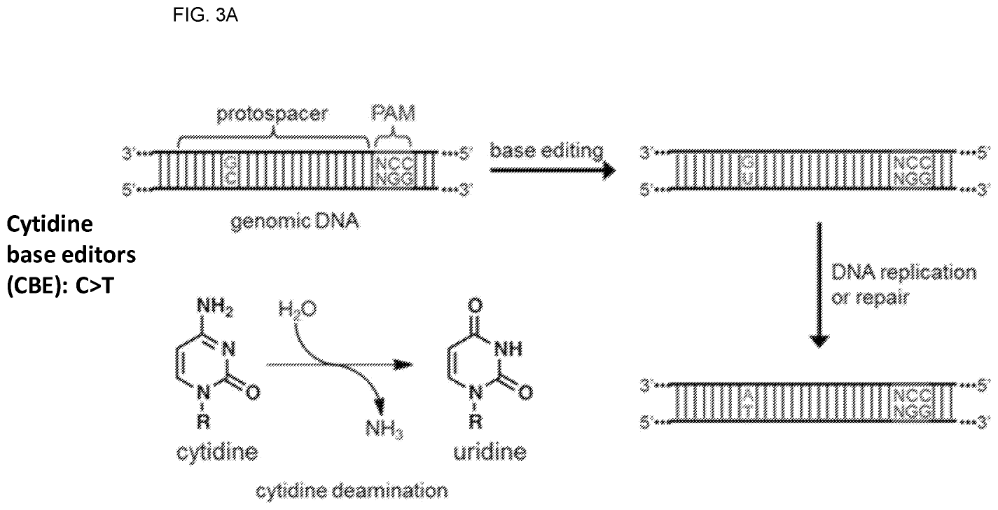

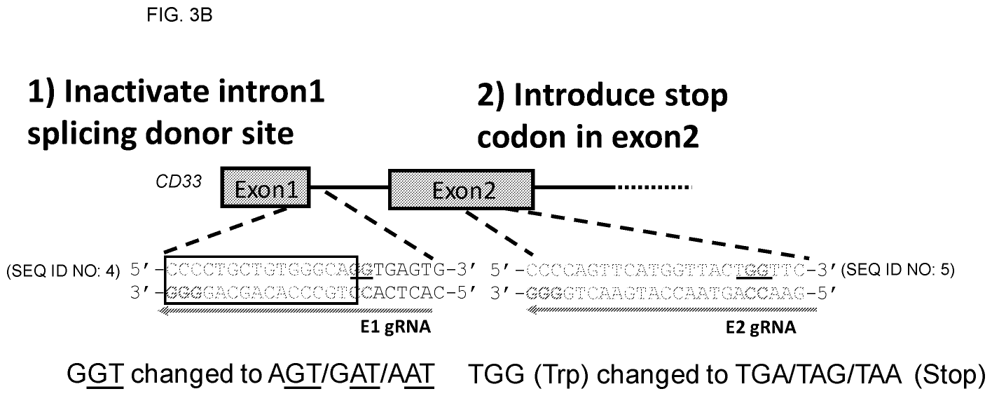

In various embodiments, a base editing system inactivates a splicing donor site of an endogenous CD33 gene, e.g., an intron 1 splicing donor site. In various embodiments, a base editing system introduces a stop codon within a CD33 coding sequence, e.g., a stop codon in a nucleic acid sequence encoding exon 2 of CD33. Particular examples of base editing systems (including gRNAs) that inactivate an intron 1 splicing donor site of CD33 are shown in FIG. 3B. Particular examples of gRNAs that introduce a stop codon into exon 2 of CD33 are shown in FIG. 3B.

Table 1 provides exemplary gRNA sequences useful in CD33 inactivation. These gRNAs are used to target two locations in the exon 2 splice acceptor site (SEQ ID NOs: 19 and 20), and two locations in the exon 3 splice acceptor site (SEQ ID NOs: 21 and 22). The positions for edits are shown bold.

TABLE 1
Representative gRNAs for CD33 knockdown

| | | | SEQ ID |
|---|---|---|---|
| Name | Sequence^ | Type | NO: |
| CD33 E1 splice donor site (see | CCCCUGCUGUGGGCAGGUGAGUG | CBE | 194 |
| Example 1) | | | |
| CD33_stop_E2 (see Example 1) | CCCCAGUUCAUGGUUACUGGUUC | CBE | 195 |
| CD33E2 splice (see Example 2) | CCCCACAGGGGCCCUGGCUA | ABE | 19 |
| CD33 exon 2 splice acceptor site 2 | CCCACAGGGGCCCUGGCUAU | ARE | 20 |
| (alternative) | | | |
| CD33 exon 3 splice acceptor site 5a | CCUCACUAGACUUGACCCAC | ARE | 21 |
| (human) | | | |
| CD33 exon 3 splice acceptor site 5b | UCUCACUAGACUUGACCCAC | ARE | 22 |
| (rhesus) | | | |

The present disclosure further includes kits that include a base editing enzyme of a base editing system (and/or nucleic acids encoding the same) and a guide RNA (gRNA) of a base editing system (and/or nucleic acids encoding the same), where the base editing system inactivates expression of CD33. The kit can further include an anti-CD33 agent and/or instructions for inactivation of CD33 in one or more cells. In various embodiments, the kit can include instructions for administration or other delivery of a base editing system to a cell, system, or subject, e.g., by administration of a viral vector encoding the base editing system to a cell, system, or subject. In various embodiments, the kit can include instructions for administration or other delivery of an anti-CD33 agent to a cell, system, or subject.

Variants of the nucleic acid and amino acid sequences disclosed and referenced herein are also included.

As indicated elsewhere, variants of gene sequences can include codon optimized variants, sequence polymorphisms, splice variants, and/or mutations that do not affect the function of an encoded product to a statistically significant degree.

Variants of the protein, nucleic acid, and gene sequences disclosed herein also include sequences with at least 70% sequence identity, 80% sequence identity, 85% sequence, 90% sequence identity, 95% sequence identity, 96% sequence identity, 97% sequence identity, 98% sequence identity, or 99% sequence identity to the protein, nucleic acid, or gene sequences disclosed herein.

Variants also include nucleic acid molecules that hybridizes under stringent hybridization conditions to a sequence disclosed herein and provide the same function as the reference sequence. Exemplary stringent hybridization conditions include an overnight incubation at 42° C. in a solution including 50% formamide, SXSSC (750 mM NaCl, 75 mM trisodium citrate), 50 mM sodium phosphate (pH 7.6), 5×Denhardt's solution, 10% dextran sulfate, and 20 μg/ml denatured, sheared salmon sperm DNA, followed by washing the filters in 0.1×SSC at 50° C. Changes in the stringency of hybridization and signal detection are primarily accomplished through the manipulation of formamide concentration (lower percentages of formamide result in lowered stringency); salt conditions, or temperature. For example, moderately high stringency conditions include an overnight incubation at 37° C. in a solution including 6×SSPE (20×SSPE=3 M NaCl; 0.2 M NaH₂PO₄; 0.02 M EDTA, pH 7.4), 0.5% SDS, 30% formamide, 100 μg/ml salmon sperm blocking DNA; followed by washes at 50° C. with 1×SSPE, 0.1% SDS. In addition, to achieve even lower stringency, washes performed following stringent hybridization can be done at higher salt concentrations (e.g. 5×SSC). Variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility.

Guidance in determining which amino acid residues can be substituted, inserted, or deleted without abolishing biological activity can be found using computer programs well known in the art, such as DNASTAR™ (Madison, Wis.) software. Preferably, amino acid changes in the protein variants disclosed herein are conservative amino acid changes, i.e., substitutions of similarly charged or uncharged amino acids. A conservative amino acid change involves substitution of one of a family of amino acids which are related in their side chains.

In a peptide or protein, suitable conservative substitutions of amino acids are known to those of skill in this art and generally can be made without altering a biological activity of a resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. Co., p. 224). Naturally occurring amino acids are generally divided into conservative substitution families as follows: Group 1: Alanine (Ala), Glycine (Gly), Serine (Ser), and Threonine (Thr); Group 2: (acidic): Aspartic acid (Asp), and Glutamic acid (Glu); Group 3: (acidic; also classified as polar, negatively charged residues and their amides): Asparagine (Asn), Glutamine (Gin), Asp, and Glu; Group 4: Gln and Asn; Group 5: (basic; also classified as polar, positively charged residues): Arginine (Arg), Lysine (Lys), and Histidine (His); Group 6 (large aliphatic, nonpolar residues): Isoleucine (Ile), Leucine (Leu), Methionine (Met), Valine (Val) and Cysteine (Cys); Group 7 (uncharged polar): Tyrosine (Tyr), Gly, Asn, Gin, Cys, Ser, and Thr; Group 8 (large aromatic residues): Phenylalanine (Phe), Tryptophan (Trp), and Tyr; Group 9 (non-polar): Proline (Pro), Ala, Val, Leu, Ile, Phe, Met, and Trp; Group 11 (aliphatic): Gly, Ala, Val, Leu, and Ile; Group 10 (small aliphatic, nonpolar or slightly polar residues): Ala, Ser, Thr, Pro, and Gly; and Group 12 (sulfur-containing): Met and Cys. Additional information can be found in Creighton (1984) Proteins, W.H. Freeman and Company.

In making such changes, the hydropathic index of amino acids may be considered. The importance of the hydropathic amino acid index in conferring interactive biologic function on a protein is generally understood in the art (Kyte and Doolittle, 1982, J. Mol. Biol. 157(1), 105-32). Each amino acid has been assigned a hydropathic index on the basis of its hydrophobicity and charge characteristics (Kyte and Doolittle, 1982). These values are: Ile (+4.5); Val (+4.2); Leu (+3.8); Phe (+2.8); Cys (+2.5); Met (+1.9); Ala (+1.8); Gly (−0.4); Thr (−0.7); Ser (−0.8); Trp (−0.9); Tyr (−1.3); Pro (−1.6); His (−3.2); Glutamate (−3.5); Gln (−3.5); aspartate (−3.5); Asn (−3.5); Lys (−3.9); and Arg (−4.5).

It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within ±2 is preferred, those within ±1 are particularly preferred, and those within ±0.5 are even more particularly preferred. It is also understood in the art that the substitution of like amino acids can be made effectively on the basis of hydrophilicity.

As detailed in U.S. Pat. No. 4,554,101, the following hydrophilicity values have been assigned to amino acid residues: Arg (+3.0); Lys (+3.0); aspartate (+3.0±1); glutamate (+3.0±1); Ser (+0.3); Asn (+0.2); Gln (+0.2); Gly (0); Thr (−0.4); Pro (−0.5±1); Ala (−0.5); His (−0.5); Cys (−1.0); Met (−1.3); Val (−1.5); Leu (−1.8); Ile (−1.8); Tyr (−2.3); Phe (−2.5); Trp (−3.4). It is understood that an amino acid can be substituted for another having a similar hydrophilicity value and still obtain a biologically equivalent, and in particular, an immunologically equivalent protein. In such changes, the substitution of amino acids whose hydrophilicity values are within ±2 is preferred, those within ±1 are particularly preferred, and those within ±0.5 are even more particularly preferred.

As outlined above, amino acid substitutions may be based on the relative similarity of the amino acid side-chain substituents, for example, their hydrophobicity, hydrophilicity, charge, size, and the like.

Therapeutic and/or CD33-Expressing Cells

In various embodiments, CD33 inactivation provides a means to selectively protect therapeutic cells that typically express CD33, such as HSC and HSPC Populations, e.g., from targeting and/or elimination upon administration to a subject or system of an anti-CD33 agent. HSCs are stem cells that can give rise to all blood cell types such as the white blood cells of the immune system (e.g., virus-fighting T cells and antibody-producing B cells), platelets, and red blood cells.

In particular embodiments, HSC can be identified and/or sorted by the following marker profiles: CD34+; Lin-CD34+CD38-CD45RA-CD90+CD49f+ (HSC1); and CD34+CD38-CD45RA-CD90-CD49f+ (HSC2). Human HSC1 can be identified by the following profiles: CD34+/CD38-/CD45RA-/CD90+ or CD34+/CD45RA-/CD90+ and mouse LT-HSC can be identified by Lin-Sca1+ckit+CD150+CD48-Flt3-CD34- (where Lin represents the absence of expression of any marker of mature cells including CD3, CD4, CD8, CD11 b, CD11 c, NK1.1, Gr1, and TER119). In particular embodiments, HSC are identified by a CD164+ profile. In particular embodiments, HSC are identified by a CD34+/CD164+ profile. In particular embodiments, the CD34+/CD45RA-/CD90+ HSC population is selected. For additional information regarding HSC marker profiles, see WO2017/218948.

HSCs can differentiate into HSPCs. HSPCs can self-renew or can differentiate into (i) myeloid progenitor cells which ultimately give rise to monocytes and macrophages, neutrophils, basophils, eosinophils, erythrocytes, megakaryocytes/platelets, or dendritic cells; or (ii) lymphoid progenitor cells which ultimately give rise to T-cells, B-cells, and lymphocyte-like cells called natural killer cells (NK-cells). For a general discussion of hematopoiesis and HSPC differentiation, see Chapter 17, Differentiated Cells and the Maintenance of Tissues, Alberts et al., 1989, Molecular Biology of the Cell, 2nd Ed., Garland Publishing, New York, N.Y.; Chapter 2 of Regenerative Medicine, Department of Health and Human Services, Aug. 5, 2006, and Chapter 5 of Hematopoietic Stem Cells, 2009, Stem Cell Information, Department of Health and Human Services.

HSPCs can be positive for a specific marker expressed in increased levels on HSPCs relative to other types of hematopoietic cells. For example, such markers include CD34, CD43, CD45RO, CD45RA, CD59, CD90, CD109, CD117, CD133, CD166, HLA DR, or a combination thereof. Also, the HSPCs can be negative for an expressed marker relative to other types of hematopoietic cells. For example, such markers include Lin, CD38, or a combination thereof. Preferably, HSPCs are CD34+.

HSCs and HSPCs sources include umbilical cord blood, placental blood, bone marrow and peripheral blood (see U.S. Pat. Nos. 5,004,681; 7,399,633; and U.S. Pat. No. 7,147,626; Craddock et al., Blood. 90(12):4779-4788 (1997); Jin et al., Bone Marrow Transplant. 42(9):581-588 (2008); Jin et al., Bone Marrow Transplant. 42(7):455-459 (2008); Pelus, Curr. Opin. Hematol. 15(4):285-292 (2008); Papayannopoulou et al., Blood. 91:2231-2239 (1998); Tricot et al., Haematologica. 93(11):1739-1742 (2008); and Weaver et al., Bone Marrow Transplant. 27: S23-S29 (2001)), as well as fetal liver, and embryonic stem cells (ESC) and induced pluripotent stem cells (iPSCs) that can be differentiated into HSC. Methods regarding collection, anti-coagulation and processing, etc. of blood and tissue samples are well known in the art. See, for example, Alsever et al., J. Med. 41:126 (1941); De Gowin et al., J. Am. Med. Assoc. 114-:850 (1940); Smith et al., J. Thome. Cardiovasc. Surg. 38:573 (1959); Rous and Turner, J. Exp. Med. 23(2): 219-237 (1916); and Hum, Calif. Med. 108(3):218-224 (1968). Stem cell sources of HSCs and HSPCs also include aortal-gonadal-mesonephros derived cells, lymph, liver, thymus, and spleen from age-appropriate donors. All collected stem cell sources of HSCs and HSPCs can be screened for undesirable components and discarded, treated, or used according to accepted current standards at the time. These stem cell sources can be steady state/naïve or primed with mobilizing or growth factor agents.

In order to avoid surgical procedures to perform a bone marrow harvest to isolate HSCs or HSPCs, approaches that harvest stem cells from the peripheral blood can be preferred. Mobilization is a process whereby stem cells are stimulated out of the bone marrow (BM) niche into the peripheral blood (PB), and likely proliferate in the PB. Mobilization allows for a larger frequency of stem cells within the PB minimizing the number of days of apheresis, reaching target number collection of stem cells, and minimizing discomfort to the donor. Agents that enhance mobilization can either enhance proliferation in the PB, or enhance migration from the BM to PB, or both. Various mobilizing agents are described herein and/or known to those of skill in the art. et al. et al.

HSC and/or HSPC can be collected and isolated from a sample using any appropriate technique. Appropriate collection and isolation procedures include magnetic separation; fluorescence activated cell sorting (FACS; Williams et al., Dev. Biol. 112(1):126-134, 1985; Lu et al., Exp. Hematol. 14(10):955-962, 1986; Lu et al., Blood. 68(1):126-133, 1986); nanosorting based on fluorophore expression; affinity chromatography; cytotoxic agents joined to a monoclonal antibody or used in conjunction with a monoclonal antibody, e.g., complement and cytotoxins; “panning” with an antibody attached to a solid matrix; selective agglutination using a lectin such as soybean (Reisner et al., Lancet. 2(8208-8209): 1320-1324, 1980); immunomagnetic bead-based sorting or combinations of these techniques, etc. These techniques can also be used to assay for successful engraftment or manipulation of hematopoietic cells in vivo, for example for gene transfer, genetic editing or cell population expansion.

In particular embodiments, it is important to remove contaminating cell populations that would interfere with isolation of the intended cell population, such as red blood cells. Removing includes both biochemical and mechanical methods to remove the undesired cell populations. Examples include lysis of red blood cells using detergents, hetastarch, hetastarch with centrifugation, cell washing, cell washing with density gradient, Ficoll-hypaque, Sepx, Optipress, filters, and other protocols that have been used both in the manufacture of HSC and/or gene therapies for research and therapeutic purposes.

In particular embodiments, a sample can be processed to select/enrich for CD34+ cells using anti-CD34 antibodies directly or indirectly conjugated to magnetic particles in connection with a magnetic cell separator, for example, the CliniMACS® Cell Separation System (Miltenyi Biotec, Bergisch Gladbach, Germany). See also, sec. 5.4.1.1 of U.S. Pat. No. 7,399,633 which describes enrichment of CD34+ HSC/HSPC from 1-2% of a normal bone marrow cell population to 50-80% of the population. HSC can also be selected to achieve the HSC profiles noted above, such as CD34+/CD45RA−/CD90+ or CD34+/CD38−/CD45RA-/CD90+.

Similarly, HSPC expressing CD43, CD45RO, CD45RA, CD59, CD90, CD109, CD117, CD133, CD166, HLA DR, or a combination thereof, can be enriched for using antibodies against these antigens. U.S. Pat. No. 5,877,299 describes additional appropriate hematopoietic antigens that can be used to isolate, collect, and enrich HSPC cells from samples.

Following isolation and/or enrichment, HSC or HSPC can be expanded in order to increase the number of HSC/HSPC. Isolation and/or expansion methods are described in, for example, U.S. Pat. Nos. 7,399,633 and 5,004,681; US Patent Publication No. 2010/0183564; International Patent Publications No. WO 2006/047569; WO 2007/095594; WO 2011/127470; and WO 2011/127472; Varnum-Finney et al., Blood 101:1784-1789, 1993; Delaney et al., Blood 106:2693-2699, 2005; Ohishi et al., J. Clin. Invest. 110:1165-1174, 2002; Delaney et al., Nature Med. 16(2): 232-236, 2010; and Chapter 2 of Regenerative Medicine, Department of Health and Human Services, August 2006, and the references cited therein. Each of the referenced methods of collection, isolation, and expansion can be used in particular embodiments of the disclosure.

Particular methods of expanding HSC/HSPC include expansion with a Notch agonist. For information regarding expansion of HSC/HSPC using Notch agonists, see sec. 5.1 and 5.3 of U.S. Pat. Nos. 7,399,633; 5,780,300; 5,648,464; 5,849,869; and 5,856,441; WO 1992/119734; Schlondorfiand & Blobel, J. Cell Sci. 112:3603-3617, 1999; Olkkonen and Stenmark, Int. Rev. Cytol. 176:1-85, 1997; Kopan et al., Cell 137:216-233, 2009; Rebay et al., Cell 67:687-699, 1991 and Jarriault et al., Mol. Cell. Biol. 18:7423-7431, 1998.

Additional culture conditions can include expansion in the presence of one or more growth factors, such as: angiopoietin-like proteins (Angptls, e.g., Angptl2, Angptl3, Angptl7, Angpt15, and Mfap4); erythropoietin; fibroblast growth factor-1 (FGF-1); Flt-3 ligand (Flt-3L); G-CSF; GM-CSF; insulin growth factor-2 (IGF-2); interleukin-3 (IL-3); interleukin-6 (IL-6); interleukin-7 (IL-7); interleukin-11 (IL-11); stem cell factor (SCF; also known as the c-kit ligand or mast cell growth factor); thrombopoietin (TPO); and analogs thereof (wherein the analogs include any structural variants of the growth factors having the biological activity of the naturally occurring growth factor; see, e.g., WO 2007/1145227 and U.S. Patent Publication No. 2010/0183564).

As a particular example for expanding HSC/HSPC, the cells can be cultured on a plastic tissue culture dish containing immobilized Delta ligand and fibronectin and 50 ng/ml of each of SCF, Flt-3L and TPO.

Cells can be autologous or allogeneic in reference to a particular subject. In particular embodiments, the cells are part of an allograft. The cells, formulations, kits, and methods disclosed herein can be used to protect normal hematopoiesis from the effects of anti-CD33 therapies.

Engineering of Therapeutic and/or CD33-Expressing Cells

Cells can be genetically modified, e.g., to achieve inactivation of CD33, using any method known in the art. A wide variety of reagents and techniques are known in the art for the introduction of heterologous nucleic acid sequences (e.g., a nucleic acid sequence encoding a base editor and/or a gRNA, e.g., for inactivation of CD33) and can be applied in vitro, ex vivo, and/or in vivo.

Particular embodiments use a genetic construct or vector to deliver base editing components and optional therapeutic gene(s) in cells. A genetic construct is an artificially produced combination of nucleotides to express particular intended molecules.

Vectors include, e.g., plasmids, cosmids, viruses, and phage. Viral vectors refer to nucleic acid molecules that include virus-derived nucleic acid elements that facilitate transfer and expression of non-native genes within a cell. In particular embodiments, viral-mediated genetic modification can utilize, for example, retroviral vectors, lentiviral vectors, foamy viral vectors, adenoviral vectors, adeno-associated viral vectors, alpharetroviral vectors or gammaretroviral vectors.

In particular embodiments, retroviral vectors (see Miller et al., 1993, Meth. Enzymol. 217:581-599) can be used. In these embodiments, the gene to be expressed is cloned into the retroviral vector for its delivery into cells. In particular embodiments, a retroviral vector includes all of the cis-acting sequences necessary for the packaging and integration of the viral genome in the target cell, i.e., (a) a long terminal repeat (LTR), or portions thereof, at each end of the vector; (b) primer binding sites for negative and positive strand DNA synthesis; and (c) a packaging signal, necessary for the incorporation of genomic RNA into virions. More detail about retroviral vectors can be found in Boesen et al., 1994, Biotherapy 6:291-302; Clowes et al., 1994, J. Clin. Invest. 93:644-651; Kiem et al., 1994, Blood 83:1467-1473; Salmons and Gunzberg, 1993, Human Gene Therapy 4:129-141; and Grossman and Wilson, 1993, Curr. Opin. in Genetics and Devel. 3:110-114.

Lentiviral vectors or “lentivirus” refers to a genus of retroviruses that are capable of infecting dividing and non-dividing cells and typically produce high viral titers. Lentiviral vectors have been employed in gene therapy for a number of diseases. For example, hematopoietic gene therapies using lentiviral vectors or gammaretroviral vectors have been used for x-linked adrenoleukodystrophy and β-thalassemia. Several examples of lentiviruses include HIV (including HIV type 1, and HIV type 2); equine infectious anemia virus; feline immunodeficiency virus (FIV); bovine immune deficiency virus (BIV); and simian immunodeficiency virus (SIV).

In particular embodiments, other retroviral vectors can be used in the practice of the methods of the invention. These include, e.g., vectors based on human foamy virus (HFV) or other viruses in the Spumavirus genera.

Foamy viruses (FVes) are the largest retroviruses known today and are widespread among different mammals, including all non-human primate species, however they are absent in humans. This complete apathogenicity qualifies FV vectors as ideal gene transfer vehicles for genetic therapies in humans and clearly distinguishes FV vectors as gene delivery system from HIV-derived and also gammaretrovirus-derived vectors.

FV vectors are also suitable for gene therapy applications because they can (1) accommodate large transgenes (>9 kb), (2) transduce slowly dividing cells efficiently, and (3) integrate as a provirus into the genome of target cells, thus enabling stable long-term expression of the transgene(s). FV vectors do need cell division for the pre-integration complex to enter the nucleus, however the complex is stable for at least 30 days and still infective. The intracellular half-life of the FV pre-integration complex is comparable to the one of lentiviruses and significantly higher than for gammaretroviruses, therefore FVes are also, similar to lentivirus vectors, able to transduce rarely dividing cells. FV vectors are natural self-inactivating vectors and characterized by the fact that they seem to have hardly any potential to activate neighboring genes. In addition, FV vectors can enter any cells known (although the receptor is not identified yet) and infectious vector particles can be concentrated 100-fold without loss of infectivity due to a stable envelope protein. FV vectors achieve high transduction efficiency in pluripotent hematopoietic stem cells and have been used in animal models to correct monogenetic diseases such as leukocyte adhesion deficiency (LAD) in dogs and FA in mice. FV vectors are also used in preclinical studies of β-thalassemia.

Point mutations can be made in FVes to render them integration incompetent. For example, foamy viruses can be rendered integration incompetent by introducing point mutations into the highly conserved DD35E catalytic core motif of the foamy virus integrase sequence. See, for example, Deyle et al., J. Virol. 84(18): 9341-9349, 2010. As another example, an FV vector can be rendered integration deficient by introducing point mutations into the Pol gene of the FV vector. Representative FV Pol coding sequence (SEQ ID NO: 23) and FV Pol amino acid sequence (SEQ ID NO: 24) with indicated nucleotides (position 2636 A to C or position 2807 A to C) or amino acid residues (D to A at 879 or at 936), respectively, that can be mutated to render the FV vector integration deficient are provided.

Adenoviral vectors are an example of vectors that can be administered in concert with HSPC mobilization. In particular embodiments, administration of an adenoviral vector occurs concurrently with administration of one or more mobilization factors. In particular embodiments, administration of an adenoviral vector follows administration of one or more mobilization factors. In particular embodiments, administration of an adenoviral vector follows administration of a first one or more mobilization factors and occurs concurrently with administration of a second one or more mobilization factors.

In particular embodiments, adenoviruses (e.g., adenovirus 5 (Ad5), adenovirus 35 (Ad35), adenovirus 11 (Ad11), adenovirus 26 (Ad26), Ad5/35++, and helper-dependent forms thereof (e.g., helper-dependent Ad5/35++ or helper dependent Ad35), adeno-associated viruses (AAV; see, e.g., U.S. Pat. No. 5,604,090), and alphaviruses can be used. See Kozarsky and Wilson, 1993, Current Opinion in Genetics and Development 3:499-503, Rosenfeld et al., 1991, Science 252:431-434; Rosenfeld et al., 1992, Cell 68:143-155; Mastrangeli et al., 1993, J. Clin. Invest. 91:225-234; Walsh et al., 1993, Proc. Soc. Exp. Bioi. Med. 204:289-300; and Lundstrom, 1999, J. Recept. Signal Transduct. Res. 19: 673-686. Additional examples of viral vectors include those derived from cytomegaloviruses (CMV), flaviviruses, herpes viruses (e.g., herpes simplex), influenza viruses, papilloma viruses (e.g., human and bovine papilloma virus; see, e.g., U.S. Pat. No. 5,719,054), poxviruses, vaccinia viruses, modified vaccinia Ankara (MVA), NYVAC, or strains derived therefrom. Other examples include avipox vectors, such as a fowlpox vectors (e.g., FP9) or canarypox vectors (e.g., ALVAC and strains derived therefrom). As indicated, helper dependent forms of viral vectors may also be used.

Methods of using retroviral and lentiviral viral vectors and packaging cells for transducing mammalian host cells with viral particles including desired transgenes are described in, e.g., U.S. Pat. No. 8,119,772; Walchli et al., 2011, PLoS One 6:327930; Zhao et al., 2005, J. Immunol. 174:4415; Engels et al., 2003, Hum. Gene Ther. 14:1155; Frecha et al., 2010, Mol. Ther. 18:1748; and Verhoeyen et al., 2009, Methods Mol. Biol. 506:97. Retroviral and lentiviral vector constructs and expression systems are also commercially available.

Other vectors or targeted genetic engineering approaches may also be utilized. The CRISPR (Clustered Regularly Interspaced Short Palindromic Repeats)/Cas (CRISPR-associated protein) nuclease system is an engineered nuclease system used for genetic engineering that is based on a bacterial system. Information regarding CRISPR-Cas systems and components thereof are described in, for example, U.S. Pat. Nos. 8,697,359, 8,771,945, 8,795,965, 8,865,406, 8,871,445, 8,889,356, 8,889,418, 8,895,308, 8,906,616, 8,932,814, 8,945,839, 8,993,233 and 8,999,641 and applications related thereto; and WO2014/018423, WO2014/093595, WO2014/093622, WO2014/093635, WO2014/093655, WO2014/093661, WO2014/093694, WO2014/093701, WO2014/093709, WO2014/093712, WO2014/093718, WO2014/145599, WO2014/204723, WO2014/204724, WO2014/204725, WO2014/204726, WO2014/204727, WO2014/204728, WO2014/204729, WO2015/065964, WO2015/089351, WO2015/089354, WO2015/089364, WO2015/089419, WO2015/089427, WO2015/089462, WO2015/089465, WO2015/089473, WO2015/089486, WO2016/205711, WO2017/106657, WO2017/127807 and applications related thereto.

Particular embodiments utilize zinc finger nucleases (ZFNs) as gene editing agents. ZFNs are a class of site-specific nucleases engineered to bind and cleave DNA at specific positions. ZFNs are used to introduce double stranded breaks (DSBs) at a specific site in a DNA sequence which enables the ZFNs to target unique sequences within a genome in a variety of different cells. For additional information regarding ZFNs and ZFNs useful within the teachings of the current disclosure, see, e.g., U.S. Pat. Nos. 6,534,261; 6,607,882; 6,746,838; 6,794,136; 6,824,978; 6,866,997; 6,933,113; 6,979,539; 7,013,219; 7,030,215; 7,220,719; 7,241,573; 7,241,574; 7,585,849; 7,595,376; 6,903,185; 6,479,626; US 2003/0232410 and US 2009/0203140 as well as Gaj et al., Nat Methods, 2012, 9(8):805-7; Ramirez et al., Nucl Acids Res, 2012, 40(12):5560-8; Kim et al., Genome Res, 2012, 22(7): 1327-33; Urnov et al., Nature Reviews Genetics, 2010, 11:636-646; Miller et al. Nature Biotechnol. 25, 778-785 (2007); Bibikova et al. Science 300, 764 (2003); Bibikova et al., Genetics 161, 1169-1175 (2002); Wolfe et al. Annu. Rev. Biophys. Biomol. Struct. 29, 183-212 (2000); Kim et al. Proc. Natl. Acad. Sci. USA. 93, 1156-1160 (1996); and Miller et al. The EMBO journal 4, 1609-1614 (1985).

Particular embodiments can use transcription activator like effector nucleases (TALENs) as gene editing agents. TALENs refer to fusion proteins including a transcription activator-like effector (TALE) DNA binding protein and a DNA cleavage domain. TALENs are used to edit genes and genomes by inducing double DSBs in the DNA, which induce repair mechanisms in cells. Generally, two TALENs must bind and flank each side of the target DNA site for the DNA cleavage domain to dimerize and induce a DSB. For additional information regarding TALENs, see U.S. Pat. Nos. 8,440,431; 8,440,432; 8,450,471; 8,586,363; and 8,697,853; as well as Joung and Sander, Nat Rev Mol Cell Biot, 2013, 14(I):49-55; Beurdeley et al., Nat Commun, 2013, 4: 1762; Scharenberg et al., Curr Gene Ther, 2013, 13(4):291-303; Gaj et al., Nat Methods, 2012, 9(8):805-7; Miller et al. Nature biotechnology 29, 143-148 (2011); Christian et al. Genetics 186, 757-761 (2010); Boch et al. Science 326, 1509-1512 (2009); and Moscou, & Bogdanove, Science 326, 1501 (2009).

Particular embodiments can utilize MegaTALs as gene editing agents. MegaTALs have a single chain rare-cleaving nuclease structure in which a TALE is fused with the DNA cleavage domain of a meganuclease. Meganucleases, also known as homing endonucleases, are single peptide chains that have both DNA recognition and nuclease function in the same domain. In contrast to the TALEN, the megaTAL only requires the delivery of a single peptide chain for functional activity.

Other methods of gene delivery include use of artificial chromosome vectors such as mammalian artificial chromosomes (Vos, Curr. Opin. Genet. Dev. 8(3): 351-359, 1998) and yeast artificial chromosomes (YAC); liposomes (Tarahovsky and Ivanitsky, Biochemistry(Mosc) 63:607-618, 1998); ribozymes (Branch and Klotman, Exp. Nephrol. 6:78-83, 1998); and triplex DNA (Chan and Glazer, 1997, J. Mol. Med. 75:267-282). YAC are typically used when the inserted nucleic acids are too large for more conventional vectors (e.g., greater than 12 kb).

When targeted genome editing approaches are utilized, genes can be inserted within genomic safe harbors. Genomic safe harbor sites are intragenic or extragenic regions of the genome that are able to accommodate the predictable expression of newly integrated DNA, generally without adverse effects on the host cell. A useful safe harbor can permit sufficient transgene expression to yield desired levels of the encoded molecule. In various embodiments, a genomic safe harbor site does not alter cellular functions. Methods for identifying genomic safe harbor sites are described in Sadelain et al., Nature Reviews (2012); 12:51-58; and Papapetrou et al., Nat Biotechnol. (1):73-8, 2011. In particular embodiments, a genomic safe harbor site meets one or more (one, two, three, four, or five) of the following criteria: (i) distance of at least 50 kb from the 5′ end of any gene, (ii) distance of at least 300 kb from any cancer-related gene, (iii) within an open/accessible chromatin structure (measured by DNA cleavage with natural or engineered nucleases), (iv) location outside a gene transcription unit and (v) location outside ultraconserved regions (UCRs), microRNA or long non-coding RNA of the genome.

In particular embodiments, a genomic safe harbor meets criteria described herein and also demonstrates a 1:1 ratio of forward:reverse orientations of lentiviral integration further demonstrating the loci does not impact surrounding genetic material.

Particular genomic safe harbors sites include CCRS, HPRT, AAVS1, Rosa and albumin. See also, e.g., U.S. Pat. Nos. 7,951,925 and 8,110,379; US Publication Nos. 20080159996; 201000218264; 20120017290; 20110265198; 20130137104; 20130122591; 20130177983 and 20130177960 for additional information and options for appropriate genomic safe harbor integration sites.

The vectors and genetic engineering approaches described herein are used to deliver genes to cells for expression. Therapeutically effective amounts of vectors (e.g., viral vectors) can be administered through any appropriate administration route such as by, injection, infusion, perfusion, and more particularly by administration by one or more of bone marrow, intravenous, intradermal, intraarterial, intranodal, intralymphatic, intraperitoneal injection, infusion, or perfusion).Delivery can utilize any appropriate technique, such as transfection, electroporation, microinjection, lipofection, calcium phosphate mediated transfection, infection with a viral or bacteriophage vector including the gene sequences, cell fusion, chromosome-mediated gene transfer, microcell-mediated gene transfer, spheroplast fusion, in vivo nanoparticle-mediated delivery, etc. Numerous techniques are known in the art for the introduction of foreign genes into cells (see e.g., Loeffler and Behr, Meth. Enzymol. 217:599-618, 1993; Cohen et al., Meth. Enzymol. 217:618-644, 1993; Cline, Pharmac. Ther. 29:69-92, 1985) and may be used, provided that the necessary developmental and physiological functions of the recipient cells are not unduly disrupted. The technique can provide for the stable transfer of the gene to the cell, so that the gene is expressible by the cell and, in certain instances, preferably heritable and expressible by its cell progeny.

In particular embodiments, the efficiency of integration, the size of the DNA sequence that can be integrated, and the number of copies of a DNA sequence that can be integrated into a genome can be improved by using transposons. Transposons or transposable elements include a short nucleic acid sequence with terminal repeat sequences upstream and downstream. Active transposons can encode enzymes that facilitate the excision and insertion of nucleic acid into a target DNA sequence.

A number of transposable elements have been described in the art that facilitate insertion of nucleic acids into the genome of vertebrates, including humans. Examples include Sleeping Beauty® (Regents of the University of Minnesota, Minneapolis, Minn.) (e.g., derived from the genome of salmonid fish); piggyBac® (Poseida Therapeutics, Inc. San Diego Calif.) (e.g., derived from lepidopteran cells and/or the Myotis lucifugus); mariner (e.g., derived from Drosophila); frog prince (e.g., derived from Rana pipiens); Tol2 (e.g., derived from medaka fish); TcBuster (e.g., derived from the red flour beetle Tribolium castaneum) and spinON.

In particular embodiments, vectors provide cloning sites to facilitate transfer of the polynucleotide sequences. Such vector cloning sites include at least one restriction endonuclease recognition site positioned to facilitate excision and insertion, in reading frame, of polynucleotide segments. Any of the restriction sites known in the art can be utilized. Most commercially available vectors already contain multiple cloning site (MCS) or polylinker regions. In addition, genetic engineering techniques useful to incorporate new and unique restriction sites into a vector are known and routinely practiced by persons of ordinary skill in the art. A cloning site can involve as few as one restriction endonuclease recognition site to allow for the insertion or excision of a single polynucleotide fragment. More typically, two or more restriction sites are employed to provide greater control of for example, insertion (e.g., direction of insert), and greater flexibility of operation (e.g., the directed transfer of more than one polynucleotide fragment). Multiple restriction sites can be the same or different recognition sites.

In particular embodiments, the gene sequence encoding any of these sequences can have one or more restriction enzyme sites at the 5′ and/or 3′ ends of the coding sequence in order to provide for easy excision and replacement of the gene sequence encoding the sequence with another gene sequence encoding a different sequence. In particular embodiments, each of the restriction sites is unique in the vector and different from the other restriction sites. In particular embodiments, each of the restriction sites are identical to the other restriction sites.

For additional information regarding procedures for genetic modification, see Anderson, Science 256:808-813 (1992); Nabel & Feigner, TIBTECH 11:211-217 (1993); Mitani & Caskey, TIBTECH 11:162-166 (1993); Dillon, TIBTECH 11:167-175 (1993); Miller, Nature 357:455-460 (1992); Van Brunt, Biotechnology 6(10):1149-1154 (1988); Vigne, Restorative Neurology and Neuroscience 8:35-36 (1995); Kremer & Perricaudet, British Medical Bulletin 51(1):31-44 (1995); Haddada et al., in Current Topics in Microbiology and Immunology Doerfler and Bohm (eds) (1995); and Yu et al., Gene Therapy 1:13-26 (1994).

Those of skill in the art will further appreciate that a base editing enzyme and/or a gRNA can be operably linked to a regulatory sequence such as a promoter, and that many such regulatory sequences are known in the art. These regulatory sequences can be eukaryotic or prokaryotic in nature. In particular embodiments, the regulatory sequence can result in the constitutive expression of the therapeutic sequence or protein upon entry of the vector into the cell. Alternatively, the regulatory sequences can include inducible sequences. Inducible regulatory sequences are well known to those skilled in the art and are those sequences that require the presence of an additional inducing factor to result in expression of the one or more molecules. Examples of suitable regulatory sequences include binding sites corresponding to tissue-specific transcription factors based on endogenous nuclear proteins, sequences that direct expression in a specific cell type, the lac operator, the tetracycline operator and the steroid hormone operator. Any inducible regulatory sequence known to those of skill in the art may be used.

In particular embodiments, the PGK promoter is used to drive expression of a therapeutic gene. In particular embodiments, the PGK promoter is derived from the human gene encoding phosphoglycerate kinase (PGK). In particular embodiments, the PGK promoter includes binding sites for the Rap1p, Abflp, and/or Gcrlp transcription factors. In particular embodiments, the PGK promoter includes 500 base pairs: Start (0); StyI (21); NspI-SphI (40); BpmI-Eco57MI (52); BaeGI-Bme1580I (63); AgeI (111); BsmBI-SpeI (246); BssS αI (252); BIpI (274); BsrDI (285); StuI (295); BgII (301); EaeI (308); AIwNI (350); EcoO1091-PpuMI (415); BspEI (420); BsmI (432); Earl (482); End (500). A PGK promoter sequence is provided in SEQ ID NO: 25.

In particular embodiments, RNA polymerase III (also called Pol III) promoters can be used to drive expression of a therapeutic gene. Pol III transcribes DNA to synthesize ribosomal 5S rRNA, tRNA, and other small RNAs. The Pol III promoters generally have well-defined initiation and stop sites and their transcripts lack poly(A) tails. The termination signal for these promoters is defined by the polythymidine tract, and the transcript is typically cleaved after the second uridine.

Additional exemplary promoters are known in the art and include galactose inducible promoters, pGAL1, pGAL1-10, pGal4, and pGa110; cytochrome c promoter, pCYC1; and alcohol dehydrogenase 1 promoter, pADH1, EF1alpha.

A promoter can be a non-coding genomic DNA sequence, usually upstream (5′) to the relevant coding sequence, to which RNA polymerase binds before initiating transcription. This binding aligns the RNA polymerase so that transcription will initiate at a specific transcription initiation site. The nucleotide sequence of the promoter determines the nature of the enzyme and other related protein factors that attach to it and the rate of RNA synthesis. The RNA is processed to produce messenger RNA (mRNA) which serves as a template for translation of the RNA sequence into the amino acid sequence of the encoded polypeptide. The 5′ non-translated leader sequence is a region of the mRNA upstream of the coding region that may play a role in initiation and translation of the mRNA. The 3′ transcription termination/polyadenylation signal is a non-translated region downstream of the coding region that functions in the plant cell to cause termination of the RNA synthesis and the addition of polyadenylate nucleotides to the 3′ end.

Promoters can include general promoters, tissue-specific promoters, cell-specific promoters, and/or promoters specific for the cytoplasm. Promoters may include strong promoters, weak promoters, constitutive expression promoters, and/or inducible (conditional) promoters. Inducible promoters direct or control expression in response to certain conditions, signals, or cellular events. For example, the promoter may be an inducible promoter that requires a particular ligand, small molecule, transcription factor, hormone, or hormone protein in order to effect transcription from the promoter. Particular examples of promoters include the AFP (α-fetoprotein) promoter, amylase 1C promoter, aquaporin-5 (AP5) promoter, αI-antitrypsin promoter, β-act promoter, β-globin promoter, β-Kin promoter, B29 promoter, CCKAR promoter, CD14 promoter, CD43 promoter, CD45 promoter, CD68 promoter, CEA promoter, c-erbB2 promoter, COX-2 promoter, CXCR4 promoter, desmin promoter, E2F-1 promoter, human elongation factor Iα promoter (EFIα), CMV (cytomegalovirus viral) promoter, minCMV promoter, SV40 (simian virus 40) immediately early promoter, EGR1 promoter, eIF4A1 promoter, elastase-1 promoter, endoglin promoter, FerH promoter, FerL promoter, fibronectin promoter, Flt-1 promoter, GAPDH promoter, GFAP promoter, GPIIb promoter, GRP78 promoter, GRP94 promoter, HE4 promoter, hGR1/1 promoter, hNIS promoter, Hsp68 promoter, the Hsp68 minimal promoter (proHSP68), HSP70 promoter, HSV-1 virus TK gene promoter, hTERT promoter, ICAM-2 promoter, kallikrein promoter, LP promoter, major late promoter (MLP), Mb promoter, Rho promoter, MT (metallothionein) promoter, MUC1 promoter, NphsI promoter, OG-2 promoter, PGK (Phospho Glycerate kinase) promoters, PGK-1 promoter, polymerase III (Pol III) promoter, PSA promoter, ROSA promoter, SP-B promoter, Survivn promoter, SYN1 promoter, SYT8 gene promoter, TRP1 promoter, Tyr promoter, ubiquitin B promoter, WASP promoter, and the Rous Sarcoma Virus (RSV) long-terminal repeat (LTR) promoter

Promoters may be obtained as native promoters or composite promoters. Native promoters, or minimal promoters, refer to promoters that include a nucleotide sequence from the 5′ region of a given gene. A native promoter includes a core promoter and its natural 5′UTR. In particular embodiments, the 5′UTR includes an intron. Composite promoters refer to promoters that are derived by combining promoter elements of different origins or by combining a distal enhancer with a minimal promoter of the same or different origin.

In particular embodiments, the SV40 promoter includes the sequence set forth in SEQ ID NO: 26. In particular embodiments, the dESV40 promoter (SV40 promoter with deletion of the enhancer region) includes the sequence set forth in SEQ ID NO: 27. In particular embodiments, the human telomerase catalytic subunit (hTERT) promoter includes the sequence set forth in SEQ ID NO: 28. In particular embodiments, the RSV promoter derived from the Schmidt-Ruppin A strain includes the sequence set forth in SEQ ID NO: 29. In particular embodiments, the hNIS promoter includes the sequence set forth in SEQ ID NO: 30. In particular embodiments, the human glucocorticoid receptor 1A (hGR 1/Ap/e) promoter includes the sequence set forth in SEQ ID NO: 31.

In particular embodiments, promoters include wild type promoter sequences and sequences with optional changes (including insertions, point mutations or deletions) at certain positions relative to the wild-type promoter. In particular embodiments, promoters vary from naturally occurring promoters by having 1 change per 20-nucleotide stretch, 2 changes per 20-nucleotide stretch, 3 changes per 20-nucleotide stretch, 4 changes per 20-nucleotide stretch, or 5 changes per 20-nucleotide stretch. In particular embodiments, the natural sequence will be altered in 1, 2, 3, 4, 5, 6, 7, 8, 9, or 10 bases. The promoter may vary in length, including from 50 nucleotides of LTR sequence to 100, 200, 250 or 350 nucleotides of LTR sequence, with or without other viral sequence.

Some promoters are specific to a tissue or cell and some promoters are non-specific to a tissue or cell. Each gene in mammalian cells has its own promoter and some promoters can only be activated in certain cell types. A non-specific promoter, or ubiquitous promoter, aids in initiation of transcription of a gene or nucleotide sequence that is operably linked to the promoter sequence in a wide range of cells, tissues and cell cycles. In particular embodiments, the promoter is a non-specific promoter. In particular embodiments, a non-specific promoter includes CMV promoter, RSV promoter, SV40 promoter, mammalian elongation factor 1α (EF1α) promoter, β-act promoter, EGR1 promoter, eIF4A1 promoter, FerH promoter, FerL promoter, GAPDH promoter, GRP78 promoter, GRP94 promoter, HSP70 promoter, β-Kin promoter, PGK-1 promoter, ROSA promoter, and/or ubiquitin B promoter.

A specific promoter aids in cell specific expression of a nucleotide sequence that is operably linked to the promoter sequence. In particular embodiments, a specific promoter is active in a B cells, monocytic cells, leukocytes, macrophages, pancreatic acinar cells, endothelial cells, astrocytes, and/or any other cell type or cell cycle. In particular embodiments, the promoter is a specific promoter. In particular embodiments, an SYT8 gene promoter regulates gene expression in human islets (Xu et al., Nat Struct Mol Biol., 18: 372-378, 2011). In particular embodiments, kallikrein promoter regulates gene expression in ductal cell specific salivary glands. In particular embodiments, the amylase 1C promoter regulates gene expression in acinar cells. In particular embodiments, the aquaporin-5 (AP5) promoter regulates gene expression in acinar cells (Zheng and Baum, Methods Mol Biol., 434: 205-219, 2008). In particular embodiments, the B29 promoter regulates gene expression in B cells. In particular embodiments, the CD14 promoter regulates gene expression in monocytic cells. In particular embodiments, the CD43 promoter regulates gene expression in leukocytes and platelets. In particular embodiments, the CD45 promoter regulates gene expression in hematopoietic cells. In particular embodiments, the CD68 promoter regulates gene expression in macrophages. In particular embodiments, the desmin promoter regulates gene expression in muscle cells. In particular embodiments, the elastase-1 promoter regulates gene expression in pancreatic acinar cells. In particular embodiments, the endoglin promoter regulates gene expression in endothelial cells. In particular embodiments, the fibronectin promoter regulates gene expression in differentiating cells or healing tissue. In particular embodiments, the Flt-1 promoter regulates gene expression in endothelial cells. In particular embodiments, the GFAP promoter regulates gene expression in astrocytes. In particular embodiments, the GPIIb promoter regulates gene expression in megakaryocytes. In particular embodiments, the ICAM-2 promoter regulates gene expression in endothelial cells. In particular embodiments, the Mb promoter regulates gene expression in muscle. In particular embodiments, the NphsI promoter regulates gene expression in podocytes. In particular embodiments, the OG-2 promoter regulates gene expression in osteoblasts, odontoblasts. In particular embodiments, the SP-B promoter regulates gene expression in lung cells. In particular embodiments, the SYN1 promoter regulates gene expression in neurons. In particular embodiments, the WASP promoter regulates gene expression in hematopoietic cells.

In particular embodiments, the promoter is a tumor-specific promoter. In particular embodiments, the AFP promoter regulates gene expression in hepatocellular carcinoma. In particular embodiments, the CCKAR promoter regulates gene expression in pancreatic cancer. In particular embodiments, the CEA promoter regulates gene expression in epithelial cancers. In particular embodiments, the c-erbB2 promoter regulates gene expression in breast and pancreas cancer. In particular embodiments, the COX-2 promoter regulates gene expression in tumors. In particular embodiments, the CXCR4 promoter regulates gene expression in tumors. In particular embodiments, the E2F-1 promoter regulates gene expression in tumors. In particular embodiments, the HE4 promoter regulates gene expression in tumors. In particular embodiments, the LP promoter regulates gene expression in tumors. In particular embodiments, the MUC1 promoter regulates gene expression in carcinoma cells. In particular embodiments, the PSA promoter regulates gene expression in prostate and prostate cancers. In particular embodiments, the Survivn promoter regulates gene expression in tumors. In particular embodiments, the TRP1 promoter regulates gene expression in melanocytes and melanoma. In particular embodiments, the Tyr promoter regulates gene expression in melanocytes and melanoma.

In certain particular embodiments, a base editing agent and/or a base editing system of the present disclosure is present in an adenoviral vector. However, those of skill in the art will appreciate that base editing agents and/or systems of the present disclosure and nucleic acid sequences encoding the same can be present in any context or form, e.g., in a vector that is not an adenoviral vector, e.g., in a plasmid. Nucleotide sequences encoding base editing systems as disclosed herein are typically too large for inclusion in many limited-capacity vector systems, but the large capacity of adenoviral vectors permits inclusion of such sequences in adenoviral vectors and genomes of the present disclosure. Indeed, as discussed elsewhere herein, adenoviral vectors can include payloads that encode a base editing system and further encode one or more additional coding sequences. An additional advantage of adenoviral vectors and genomes as disclosed herein for gene therapy with payloads encoding base editors of the present disclosure is that adenoviral genomes do not naturally integrate into host cell genomes, which facilitates transient expression of base editing systems, which can be desirable, e.g., to avoid and/or reduce immunogenicity and/or genotoxicity.

Treatments using in viva gene therapy, which includes the direct delivery of a viral vector to a patient, have been explored. In vivo gene therapy is an attractive approach because it may not require any genotoxic conditioning (or could require less genotoxic conditioning) nor ex vivo cell processing and thus could be adopted at many institutions worldwide, including those in developing countries, as the therapy could be administered through an injection, similar to what is already done worldwide for the delivery of vaccines. In various embodiments methods of in vivo gene therapy with adenoviral vectors of the present disclosure can include one or more steps of (i) target cell mobilization, (ii) immunosuppression, (iii) administration of a vector, genome, system or formulation provided herein, and/or (iv) selection of transduced cells and/or cells that have integrated an integration element of a payload of an adenoviral vector or genome.

Adenovirus (or, interchangeably, “adenoviral”) vectors and genomes refer to those constructs containing adenovirus sequences sufficient to (a) support packaging of an expression construct and to (b) express a coding sequence. Adenoviral genomes can be linear, double-stranded DNA molecules. As those of skill in the art will appreciate, a linear genome such as an adenoviral genome can be present in circular plasmid, e.g., for viral production purposes.

Natural adenoviral genomes range from 26 kb to 45 kb in length, depending on the serotype.

Adenoviral vectors include Adenoviral DNA flanked on both ends by inverted terminal repeats (ITRs), which act as a self-primer to promote primase-independent DNA synthesis and to facilitate integration into the host genome. Adenoviral genomes also contain a packaging sequence, which facilities proper viral transcript packaging and is located on the left arm of the genome. Viral transcripts encode several proteins including early transcriptional units, E1, E2, E3, and E4 and late transcriptional units which encode structural components of the Ad virion (Lee et al., Genes Dis., 4(2):43-63, 2017).

Adenoviral vectors include adenoviral genomes. Recombinant adenoviral vectors are adenoviral vectors that include a recombinant adenoviral genome. A recombinant adenoviral vector includes a genetically engineered form of an adenovirus. Those of skill in the art will appreciate that throughout the present application disclosure of an adenoviral vector includes disclosure of the adenoviral genome thereof, and that disclosure of an adenoviral genome includes disclosure of an adenoviral vector including the disclosed adenoviral genome.

The adenovirus is a large, icosahedral-shaped, non-enveloped virus. The viral capsid includes three types of proteins including fiber, penton, and hexon based proteins. The hexon makes up the majority of the viral capsid, forming the 20 triangular faces. The penton base is located at the 12 vertices of the capsid and the fiber (also referred to as knobbed fiber) protrudes from each penton base. These proteins, the penton and fiber, are of particular importance in receptor binding and internalization as they facilitate the attachment of the capsid to a host cell (Lee et al., Genes Dis., 4(2):43-63, 2017).

Ad35 fiber is a fiber protein trimer, each fiber protein including an N-terminal tail domain that interacts with the pentameric penton base, a C-terminal globular knob domain (fiber knob) that functions as the attachment site for the host cell receptors, and a central shaft domain that connects the tail and the knob domains (shaft). The tail domain of the trimeric fiber attaches to the pentameric penton base at the 5-fold axis. In various embodiments, an Ad35 fiber knob includes amino acids 123 to 320 of a canonical wild-type Ad35 fiber protein. In various embodiments, an Ad35 fiber knob includes at least 60 amino acids (e.g., at least 60, 70, 80, 90, 100, 110, 120, 130, 140, 150, 160, 170, 180, 190, or 198 amino acids) having at least 80% (e.g., at least 80%, 85%, 90%, 95%, 96%, 97%, 98%, or 99% sequence identity) sequence identity with a corresponding fragment of amino acids 123 to 320 of a canonical wild-type Ad35 fiber protein. In various embodiments, a fiber knob is engineered for increased affinity with CD46, and/or to confer increased affinity with CD46 to a fiber protein, fiber, or vector, as compared to a reference fiber knob, fiber protein, fiber or vector including a canonical wild-type Ad35 fiber protein, optionally wherein the increase is an increase of at least 1.1-fold, e.g., at least 1, 2, 3, 4, 5, 10, 15, or 20-fold. The central shaft domain consists of 5.5 p-repeats, each containing 15-20 amino acids that code for two anti-parallel β-strands connected by a β-turn. The β-repeats connect to form an elongated structure of three intertwined spiraling strands that is highly rigid and stable.

Adenovirus is particularly suitable for use as a gene transfer vector because of its mid-sized genome, ease of manipulation, high titer, wide target-cell range and high infectivity. Both ends of the viral genome contain 100-200 base pair ITRs, which are cis elements necessary for viral DNA replication and packaging. The early (E) and late (L) regions of the genome contain different transcription units that are divided by the onset of viral DNA replication. The E1 region (E1 A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off. The products of the late genes, including the majority of the viral capsid proteins, are expressed only after significant processing of a single primary transcript issued by the major late promoter (MLP). The MLP is particularly efficient during the late phase of infection, and all the mRNAs issued from this promoter possess a 5′-tripartite leader (TPL) sequence which makes them preferred mRNAs for translation.

Among adenoviruses, there are also over 50 serotypes. Adenovirus type 5 is a human adenovirus about which a great deal of biochemical and genetic information is known, and it has historically been used for most constructions employing adenovirus as a vector. Ad5 has been widely used in gene therapy research.

Ad35 is one of the rarest of the 57 known human serotypes, with a seroprevalence of <7% and no cross-reactivity with Ad5. Ad35 is less immunogenic than Ad5, which is, in part, due to attenuation of T-cell activation by the Ad35 fiber knob. Further, after intravenous (iv) injection, there is only minimal transduction (only detectable by PCR) of tissues, including the liver, in human CD46 transgenic (hCD46tg) mice and non-human primates. First-generation Ad35 vectors have been used clinically for vaccination purposes.

The complete genome of a representative natural Ad35 adenovirus is known and publicly available (see, e.g., Gao et al., 2003 Gene Ther. 10(23): 1941-9; Reddy et al. 2003 Virology 311(2): 384-393; GenBank Accession No. AX049983). While the Ad5 genome is 35,935 bp with a G+C content of 55.2%, the Ad35 genome is 34,794 bp with a G+C content of 48.9%. The genome of Ad35 is flanked by inverted terminal repeats (ITRs). In various embodiments, Ad35 ITRS include 137 bp (e.g., a 5′ Ad35 that includes nucleotides 1-137 or 4-140 of GenBank Accession No. AX049983 and a 3′ ITR that includes nucleotides 34658-34794 of GenBank Accession No. AX049983), which are longer than those of Ad5 (103 bp). In various embodiments, an Ad35 5′ ITR includes at least 80 nucleotides (e.g., at least 80, 90, 100, 110, 120, 130, 140, 150, 160, 170, 180, 190, or 200 nucleotides, e.g., a number of nucleotides having a lower bound of 80, 90, 100, 110, 120, or 130 nucleotides and an upper bound of 130, 140, 150, 160, 170, 180, 190, or 200 nucleotides, e.g., 137 nucleotides) having at least 80% sequence identity (e.g., at least 80%, 85%, 90%, 95%, 96%, 97%, 98%, or 99% sequence identity) with a corresponding fragment of nucleotides 1-200 of GenBank Accession No. AX049983 and an Ad35 3′ ITR includes at least 80 nucleotides (e.g., at least 80, 90, 100, 110, 120, 130, 140, 150, 160, 170, 180, 190, or 200 nucleotides, e.g., a number of nucleotides having a lower bound of 80, 90, 100, 110, 120, or 130 nucleotides and an upper bound of 130, 140, 150, 160, 170, 180, 190, or 200 nucleotides, e.g., 137 nucleotides) having at least 80% sequence identity (e.g., at least 80%, 85%, 90%, 95%, 96%, 97%, 98%, or 99% sequence identity) with a corresponding fragment of nucleotides 34595-34794 of GenBank Accession No. AX049983. In various embodiments, an ITR is sufficient for one or both of Ad35 encapsidation and/or replication. In various embodiments, an Ad35 ITR sequence for Ad35 vectors differs in that the first 8 bp are CTATCTAT rather than CATCATCA (Wunderlich, J. Gen Viro. 95: 1574-1584, 2014).

In various embodiments, packaging of the adenovirus genome is mediated by a cis-acting packaging sequence domain located at the 5′ end of the viral genome adjacent to the ITR, and packaging occurs in a polar fashion from left to right. The packaging sequence of Ad35 is located at the left end of the genome with five to seven putative “A” repeats. In various embodiments, the present disclosure includes a recombinant Ad35 donor vector or genome that includes an Ad35 packaging sequence. In various embodiments, the present disclosure includes a recombinant Ad35 helper vector or genome that includes a packaging sequence flanked by recombinase sites. In various embodiments, an Ad35 packaging sequence refers to a nucleic acid sequence including nucleotides 138-481 of GenBank Accession No. AX049983 or a fragment thereof sufficient for or required for packaging of an Ad35 vector or genome (e.g., such that flanking of the sequence with recombinase sites and excision by recombination of the recombinase sites renders the vector or genome deficient for packaging, e.g., by at least 10% as compared to a reference including the packaging sequence, e.g., by at least 10%, 20%, 30%, 40$, 50%, 60%, 70%, 80%, 90%, 91%, 92%, 93%, 94%, 95%, 96%, 97%, 98%, 99%, or 100%, optionally wherein the reference includes the packaging sequence flanked by the recombines sites). In various embodiments, an Ad35 packaging sequence includes at least 80 nucleotides (e.g., at least 80, 90, 100, 110, 120, 130, 140, 150, 160, 170, 180, 190, 200, 225, 250, 275, or 300 nucleotides, e.g., a number of nucleotides having a lower bound of 80, 90, 100, 110, 120, 130, 140, or 150 nucleotides and an upper bound of 150, 160, 170, 180, 190, 200, 225, 250, 275, or 300 nucleotides) having at least 80% sequence identity (e.g., at least 80%, 85%, 90%, 95%, 96%, 97%, 98%, or 99% sequence identity) with a corresponding fragment of nucleotides 137-481 of GenBank Accession No. AX049983.

In various embodiments, an Ad35 helper vector can include recombinase sites inserted to flank a packaging sequence, where a first recombinase site is inserted immediately adjacent to (e.g., before, or after) a position selected from between nucleotide 130 and nucleotide 400 (e.g., between nucleotides 138 and 180, 138 and 200, 138 and 220, 138 and 240, 138 and 260, 138 and 280, 138 and 300, 138 and 320, 138 and 340, 138 and 360, 138 and 366, 138 and 380, or 138 and 400) and a second recombinase site inserted immediately adjacent to (e.g., after, or before) a position selected from between nucleotide 300 and nucleotide 550 (e.g., between nucleotides 344 and 360, 344 and 380, 344 and 400, 344 and 420, 344 and 440, 344 and 460, 344 and 480, 344 and 481, 344 and 500, 344 and 520, 344 and 540, or 344 and 550). Those of skill in the art will appreciate that the term packaging sequence does not necessarily include all of the packaging elements present in a given vector or genome. For example, a helper genome can include recombinase direct repeats that flank a packaging sequence, where the flanked packaging sequence does not include all of the packaging elements present in the helper genome. Accordingly, in certain embodiments, one or two recombinase direct repeats of a helper genome are positioned within a larger packaging sequence, e.g., such that a larger packaging sequence is rendered noncontiguous by introduction of the one or two recombinase direct repeats. In various embodiments, recombinase direct repeats of a helper genome flank a fragment of the packaging sequence such that excision of the flanked packaging sequence by recombination of the recombinase direct repeats reduces or eliminates (more generally, disrupts) packaging of the helper genome and/or ability of the helper genome to be packaged. By way of example, recombinase direct repeats (DRs) are positioned within 550 nucleotides of the 5′ end of the Ad35 genome in order to functionally disrupt the Ad35 packaging signal but not the 5′ Ad35 ITR. In various embodiments, the DRs are positioned closer than 550 nucleotides from the 5′ end of the Ad35 genome, for instance within 540, 530, 520, 510, 500, 495,490, 480, 470, 450, 440, 400, 380, 360 nucleotides, or closer than within 360 nucleotides of the 5′ end of the Ad35 genome, in order to functionally disrupt the Ad35 packaging signal but not the 5′ Ad35 ITR.

In various embodiments, the present disclosure includes a recombinant Ad35 donor vector or genome that includes an Ad35 5′ ITR, an Ad35 packaging sequence, and an Ad35 3′ ITR, In certain embodiments, an Ad35 5′ ITR, an Ad35 packaging sequence, and an Ad35 3′ ITR are the only fragments of the recombinant Ad35 donor vector or genome (e.g., the only fragments over 50 or over 100 base pairs) that are derived from, and/or have at least 80% identity to, a canonical Ad35 genome.

Ad35 early regions include E1A, E1B, E2A, E2B, E3, and E4. Ad35 intermediate regions include pIX and IVa2. The late transcription unit of Ad35 is transcribed from the major late promoter (MLP), located at 16.9 map units. The late mRNAs in Ad35 can be divided into five families of mRNAs (L1-L5), depending on which poly(A) signal is used by these mRNAs. Based on the MLP consensus initiator element, and splice donor and splice acceptor site sequences, the length of tripartite leader (TPL) has been predicted to be 204 nucleotides. The first leader of the TPL, which is adjacent to MLP, is 45 nucleotides in length. The second leader located within the coding region of DNA polymerase is 72 nucleotides in length. The third leader lies within the coding region of precursor terminal protein (pTP) of E2B region and is 87 nucleotides in length. While Ad5 contains two virus-associated (VA) RNA genes, only one virus-associated RNA gene occurs in the genome of Ad35. This VA RNA gene is located between the genes coding for the 52/55K L1 protein and pTP.

In particular embodiments, an Ad35++ vector is a chimeric vector with a mutant Ad35 fiber knob (e.g., a recombinant Ad35 vector with a mutant Ad35 fiber knob or an Ad5/35 vector with a mutant Ad35 fiber knob). In particular embodiments, an Ad35++ genome is a genome that encodes a mutant Ad35 fiber knob (e.g., a recombinant Ad35 helper genome encoding a mutant Ad35 fiber knob or an Ad5/35 helper genome encoding a mutant Ad35 fiber knob). In various embodiments, an Ad35++ mutant fiber knob is an Ad35 fiber knob mutated to increase the affinity to CD46, e.g., by 25-fold, e.g., such that the Ad35++ mutant fiber knob increases cell transduction efficiency, e.g., at lower multiplicity of infection (MOI) (Li and Lieber, FEBS Letters, 593(24): 3623-3648, 2019).

In various embodiments, an Ad35++ mutant fiber knob includes at least one mutation selected from Ile192Val, Asp207Gly (or Glu207Gly in certain Ad35 sequences), Asn217Asp, Thr226Ala, Thr245Ala, Thr254Pro, Ile256Leu, Ile256Val, Arg259Cys, and Arg279His. In various embodiments, an Ad35++ mutant fiber knob includes each of the following mutations: Ile192Val, Asp207Gly (or Glu207Gly in certain Ad35 sequences), Asn217Asp, Thr226Ala, Thr245Ala, Thr254Pro, Ile256Leu, Ile256Val, Arg259Cys, and Arg279His. In various embodiments, amino acid numbering of an Ad35 fiber is according to GenBank accession AP_000601 or an amino acid sequence corresponding thereto, e.g., where position 207 is Glu or Asp. In various embodiments, an Ad35 fiber has an amino acid sequence according to GenBank accession AP_000601. Further description of Ad35++ fiber knob mutations is found in Wang J. Virol. 82(21): 10567-10579, 2008, which is incorporated herein by reference in its entirety and with respect to fiber knobs.

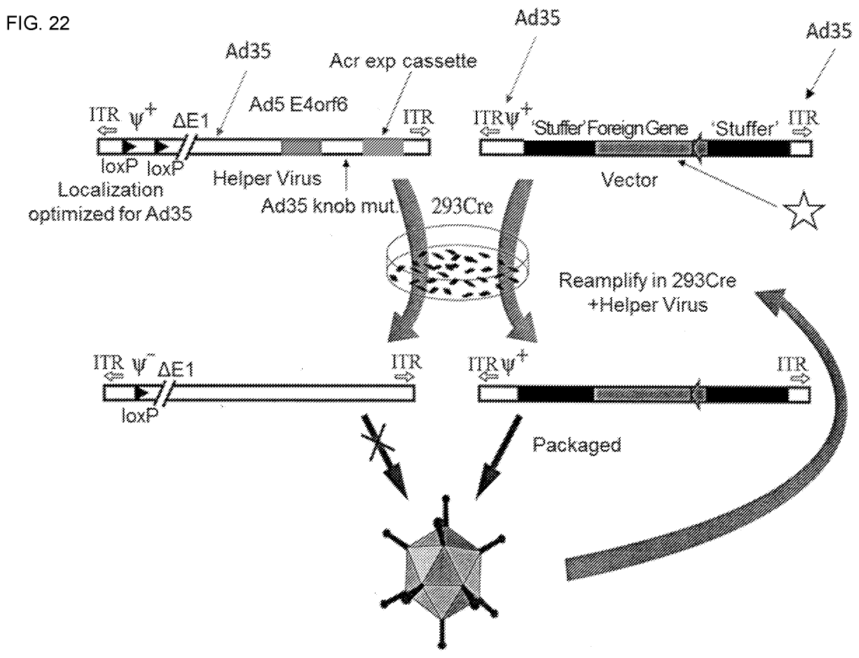

Ad5/35 vectors of the present disclosure include adenoviral vectors that include Ad5 capsid polynucleotides and chimeric fiber polynucleotides including an Ad35 fiber knob, the chimeric fiber polynucleotide typically also including an Ad35 fiber shaft (e.g., Ad5 fiber amino acids 1-44 in combination with Ad35 fiber amino acids 44-323). In various embodiments, the fiber includes an Ad35++ mutant fiber knob. In various Ad5/35 vectors of the present disclosure, all proteins except fiber knob domains and shaft were derived from serotype 5, while fiber knob domains and shafts were derived from serotype 35, and mutations that increased the affinity to CD46 were introduced into the Ad35 fiber knob (see WO 2010/120541 A2). Additionally, in various embodiments, the ITR and packaging sequence of the Ad5/35 vectors are derived from Ad5. (See Table 2 for exemplary knob mutations; and FIG. 22 for a general schematic of HDAd35 vector production.)

TABLE 2
Mutated Ad35 Knob increased binding to CD46

| | Kd (Oleks) | |
|---|---|---|
| A1: Asn217Asp Thr245Pro | A1 4.82 nM | Asp207Gly +++ |
| Ile256Leu* | | |
| A2: Asp207Gly Thr245Ala* | A2 0.629 nM | Thr245Ala ++ |
| A3: Asp207Gly Thr226Ala* | A3 1.407 nM | Ile256Leu + |
| A8: Ile192Val Ile256Val ? | A8 13.6850 nM | |
| B1: Asp207Gly* | B1 1.774 nM | |
| B2: wtAd35(207Asp) | B2 14.98 nM | |
| B3: Asn217Asp* | B3 16.85 nM | |
| B4: Thr245Ala* | B4 7.64 nM | |
| B5: Ile256Leu* | B5 10.96 nM | |
| B6: Ad3 | B6 no binding | |
| B7: Ad11 | B7 11.22 nM | |
| M1: Arg279Cys* | M1 no binding | |
| M3: Arg279His* | M3 no binding | |
| wtAd35* | 13.7 nM | |
| wtAd35* | 15.36 nM | |
| AA: Asp207Gly Thr245Ala | 0.943 nM | |
| Ile256Leu* | | |

*Published in Wang et al. (J. Virol., 82(21): 10567-10579, 2008)
**Published in Wang et al. (J. Virol. 81 (23): 12785-12792, 2007)

In general, the path from a natural adenoviral vector to a helper-dependent adenoviral vector can include three generations. First-generation adenoviral vectors are engineered to remove genes E1 and E3. Without these genes, adenoviral vectors cannot replicate on their own but can be produced in E1-expressing mammalian cell lines such as HEK293 cells. With only first-generation modifications, adenoviral vector cloning capacity is limited, and host immune response against the vector can be problematic for effective payload expression. Second-generation adenoviral vectors, in addition to E1/E3 removal, are engineered to remove non-structural genes E2 and E4, resulting in increased capacity and reduced immunogenicity. Third-generation adenoviral vector (also referred to as gutless, high capacity adenoviral vector, or helper-dependent adenoviral vector (HdAd)) are further engineered to remove all viral coding sequences, and retain only the ITRs of the genome and packaging sequence of the genome or a functional fragment thereof. Because these genomes do not encode the proteins necessary for viral production, they are helper-dependent: a helper-dependent genome can only be packaged into vector if they are present in a cell that includes a nucleic acid sequence that provides viral proteins in trans. These helper-dependent vectors are also characterized by still greater capacity and further decreased immunogenicity. Because the sequences of each viral genome are distinct at least for each serotype, the proper modifications required to produce a helper-dependent viral genome, and/or a helper genome, for a given serotype cannot be predicted from available information relating to other serotypes.

Helper-dependent adenoviral vectors (HDAd) engineered to lack all viral coding sequences can efficiently transduce a wide variety of cell types, and can mediate long-term transgene expression with negligible chronic toxicity. By deleting the viral coding sequences and leaving only the cis-acting elements necessary for genome replication (ITRs) and encapsidation (γ), cellular immune response against the Ad vector is reduced. HDAd vectors have a large cloning capacity of up to 37 kb, allowing for the delivery of large payloads. These payloads can include large therapeutic genes or even multiple transgenes and large regulatory components to enhance, prolong, and regulate transgene expression. Like other adenoviral vectors, typical HDAd genome generally remain episomal and do not integrate with a host genome (Rosewell et al., J Genet Syndr Gene Ther. Suppl 5:001, 2011, doi: 10.4172/2157-7412.s5-001).

In some HDAd vector systems, one viral genome (a helper genome) encodes all of the proteins required for replication but has a conditional defect in the packaging sequence, making it less likely to be packaged into a virion. As noted above, this can require identification of the packaging sequence or a functionally contributing (e.g., functionally required) fragment thereof and modification of the subject genome in a manner that does not negate propagation of the helper vector, which cannot be ascertained from existing knowledge relating to other adenoviral serotypes, A separate donor viral genome includes (e.g., only includes) viral ITRs, a payload (e.g., a therapeutic payload), and a functional packaging sequence (e.g., normal wild-type packaging sequence, or a functional fragment thereof), which allows this donor viral genome to be selectively packaged into HDAd viral vectors and isolated from the producer cells. HDAd donor vectors can be further purified from helper vectors by physical means. In general, some contamination of helper vectors and/or helper genomes in HDAd viral vectors and HDAd viral vector formulations can occur and can be tolerated.

In some HDAd vector systems, a helper genome utilizes a Cre/loxP system. In certain such HDAd vector systems, the HDAd donor genome includes 500 bp of noncoding adenoviral DNA that includes the adenoviral ITRs which are required for genome replication, and ψ which is the packaging sequence or a functional fragment thereof required for encapsidation of the genome into the capsid. It has also been observed that the HDAd donor vector genome can be most efficiently packaged when it has a total length of 27.7 kb to 37 kb, which length can be composed, e.g., of a therapeutic payload and/or a “stuffer” sequence. The HDAd donor genome can be delivered to cells, such as 293 cells (HEK293) that expresses Cre recombinase, optionally where the HDAd donor genome is delivered to the cells in a non-viral vector form, such as a bacterial plasmid form (e.g., where the HDAd donor genome is constructed as a bacterial plasmid (pHDAd) and is liberated by restriction enzyme digestion). The same cells can be transduced with the helper genome, which can include an E1-deleted Ad vector bearing a packaging sequence or functionally contributing (e.g., functionally required) fragment thereof flanked by loxP sites so that following infection of 293 cells expressing Cre recombinase, the packaging sequence or functionally contributing (e.g., functionally required) fragment thereof is excised from the helper genome by Cre-mediated site-specific recombination between the loxP sites. Thus, the HDAd donor genome can be transfected into 293 cells (HEK293) that express Cre and are transduced with a helper genome bearing a packaging sequence (γ) or a functional fragment thereof flanked by recombinase sites (e.g., loxP sites) such that excision mediated by a corresponding recombinase (e.g., Cre-mediated excision) of γ renders the helper virus genome unpackageable, but still able to provide all of the necessary trans-acting factors for propagation of the HDAd. After excision of the packaging sequence or functionally contributing (e.g., functionally required) fragment thereof, a helper genome is unpackageable but still able to undergo DNA replication and thus trans-complement the replication and encapsidation of the HDAd donor genome. In some embodiments, to prevent generation of replication competent Ad (RCA; E1+) as a consequence of homologous recombination between the helper and HDAd donor genomes present in 293 cells (HEK293) a “stuffer” sequence can be inserted into the E3 region to render any E1+ recombinants too large to be packaged. Similar HDAd production systems have been developed using FLP (e.g., FLPe)/frt site-specific recombination, where FLP-mediated recombination between frt sites flanking the packaging sequence of the helper genome selects against encapsidation of helper genomes in 293 cells (HEK293) that express FLP. Alternative strategies to select against the helper vectors have been developed. An Ad35 helper virus typically includes all of the viral genes except for those in E1, as E1 expression products can be supplied by complementary expression from the genome of a producer cell line.

HDAd5/35 donor vectors, donor genomes, helper vectors and helper genomes are exemplary of compositions provided herein and used in various methods of the present disclosure. An HDAd5/35 vector or genome is a helper-dependent chimeric Ad5/35 vector or genome with an Ad35 fiber knob and an Ad5 shaft. An HDAd5/35++ vector or genome is a helper-dependent chimeric Ad5/35 vector or genome with a mutant Ad35 fiber knob. The vector is mutated to increase the affinity to CD46, e.g., by 25-fold and increases cell transduction efficiency at lower multiplicity of infection (MOI) (Li & Lieber, FEBS Letters, 593(24): 3623-3648, 2019). An Ad5/35 helper vector is a vector that includes a helper genome that includes a conditionally expressed (e.g., frt-site or loxP-site flanked) packaging sequence and encodes all of the necessary trans-acting factors for production of Ad5/35 virions into which the donor genome can be packaged.

HDAd35 donor vectors, donor genomes, helper vectors and helper genomes are also exemplary of compositions provided herein and used in various methods of the present disclosure. Related application No. PCT/US2020/040756 is incorporated herein by reference in its entirety and with respect to adenoviral vectors, in particular with respect to Ad35 vectors, including HDAd35 vectors and related vectors. An HDAd35 vector or genome is a helper-dependent Ad35 vector or genome. An HDAd35++ vector or genome is a helper-dependent Ad35 vector or genome with a mutant Ad35 fiber knob which enhances its affinity to CD46 and increases cell transduction efficiency. An Ad35 helper vector is a vector that includes a helper genome that includes a conditionally expressed (e.g., frt-site or loxP-site flanked) packaging sequence and encodes all of the necessary trans-acting factors for production of Ad35 virions into which the donor genome can be packaged. The present disclosure further includes an HDAd35 donor vector production system including a cell including an HDAd35 donor genome and an Ad35 helper genome. In certain such cells, viral proteins encoded and expressed by the helper genome can be utilized in production of HDAd35 donor vectors in which the HDAd35 donor genome is packaged. Accordingly, the present disclosure includes methods of production of HDAd35 donor vectors by culturing cells that include an HDAd35 donor genome and an Ad35 helper genome. In some embodiments the cells encode and express a recombinase that corresponds to recombinase direct repeats that flank a packaging sequence of the Ad35 helper vector. In some embodiments, the flanked packaging sequence of the Ad35 helper genome has been excised.

In some embodiments the Ad35 helper genome encodes all Ad35 coding sequences. In some embodiments the Ad35 helper genome encodes and/or expresses all Ad35 coding sequences except for one or more coding sequences of the E1 region and/or an E3 coding sequence and/or an E4 coding sequence. In various embodiments, a helper genome that does not encode and/or express an Ad35 E1 gene does not encode and/or express an Ad35 E4 gene, optionally wherein the Ad35 helper genome is further engineered to include an Ad5 E4orf6 coding sequence. In various embodiments, as will be appreciate by those of skill in the art, cells of compositions and methods for production of HDAd 35 donor vectors can be cells that express an Ad5 E1 expression product. In various embodiments, as will be appreciate by those of skill in the art, cells of compositions and methods for production of HDAd 35 donor vectors can be 293 T cells (HEK293).

A helper may be engineered from wild-type or similarly propagation-competent vectors, such as a wild-type or propagation-competent Ad5 vector or Ad35 vector. As those of skill in the art will appreciate, one strategy that can be used in engineering of a helper vector is deletion or other functional disruption of E1 gene expression. The E1 region, located in the 5′ portion of adenoviral genomes, encodes proteins required for wild-type expression of the early and late genes. E1 deletion reduces or eliminates expression of certain viral genes controlled by E1, and E1-deleted helper viruses are replication-defective. Accordingly, E1-deficient helper virus can be propagated using cell lines that express E1. For example, where an E1-deficient Ad35 helper vector is engineered to encode an Ad5 E4orf6, the helper vector can be propagated in a cell line that expresses Ad5 E1, and where an E1-deficient Ad35 helper vector encodes an Ad5 E4orf6, the helper vector can be propagated in a cell line that expresses Ad5 E1. In one exemplary cell type for HDAd35 vector production, HEK293 cells express Ad5 E1b55k, which is known to form a complex with Ad5 E4 protein ORF6. Table 3 provides an example summary of expression products encoded by an Ad35 genome (see Gao, Gene Ther. 10:1941-1949, 2003).

TABLE 3
Predicated translational features of the Ad35 genome.

| | | |
|---|---|---|
| Features | From | To |

E1 and pIX regions

| | | | |
|---|---|---|---|
| E1A 261R | | 569 | 1148 |
| | Join | 1233 | 1441 |
| E1A 230R | | 569 | 1055 |
| | Join | 1233 | 1441 |
| E1A 58R | | 569 | 640 |
| | Join | 1233 | 1337 |
| E1B 214R (small T antigen) | | 1611 | 2153 |
| E1B 494R (large T antigen) | | 1916 | 3400 |
| pIX | | 3484 | 3903 |
| ORF-1 | | 2366 | 2689 |

E2 and IVa2 regions (complementary strand)

| | | | |
|---|---|---|---|
| IVa2 | | 5579 | 5590 |
| | Join | 3966 | 5300 |
| E2B DNA pol | | 5069 | 8437 |
| E2B pTP | | 8440 | 10356 |
| E2A DBF | | 22414 | 23415 |
| ORF-2 | | 5988 | 6482 |
| ORF-3 | | 7847 | 8257 |
| ORF-4 | | 15663 | 15971 |
| ORF-5 | | 15743 | 16216 |
| ORF-6 | | 16457 | 17041 |
| ORF-7 | | 17543 | 17938 |
| ORR-8 | | 17994 | 18713 |
| ORF-9 | | 21858 | 22436 |
| ORF-10 | | 22128 | 22502 |
| ORF-11 | | 23027 | 23488 |

E3 region

| | | |
|---|---|---|
| E3 12.2K protein | 27198 | 27515 |
| E3 15.0K protein | 27469 | 27864 |
| E3 18.5K protein | 27849 | 28349 |
| E3 20.3K protein | 28369 | 28914 |
| E3 20.6K protein | 28932 | 29495 |
| E3 15.2K protein | 29817 | 30221 |
| E3 15.3K protein | 30214 | 30621 |
| ORF-12 | 25693 | 26019 |
| ORF-13 | 27908 | 28240 |

E4 region (complementary strand)

| | | |
|---|---|---|
| E4 299R | 32075 | 32974 |
| E4 145R | 33604 | 34041 |
| E4 125R | 34038 | 34415 |
| E4 117R | 33254 | 33607 |
| E4 122R | 32877 | 33245 |
| ORF-14 | 33100 | 33609 |
| VA RNA region | 10433 | 10594 |

L region

| | | | |
|---|---|---|---|
| L1 52, 55K | | 10653 | 11819 |
| L1 IIIa | | 11845 | 13608 |
| L2 III (penton base) | | 13690 | 15375 |
| L2 pVII | | 15383 | 15961 |
| L2 V | | 16004 | 17059 |
| L3 pVI | | 17399 | 18139 |
| L3 II (hexon) | | 18255 | 21113 |
| L3 23K (protease) | | 21150 | 21779 |
| L4 100K | | 23446 | 25884 |
| L4 22K | | 25616 | 26191 |
| L4 33K | | 25616 | 25934 |
| | Join | 26104 | 26465 |
| L4 pVIII | | 26515 | 27198 |
| L5 IV(fiber) | | 30826 | 31797 |

The present disclosure includes, among other things, HDAd35 donor vectors and genomes that include Ad35 ITRs (e.g., a 5′ Ad35 ITR and a 3′ ITR), e.g., where two Ad35 ITRs flank a payload. The present disclosure includes, among other things, HDAd35 donor vectors and genomes that include an Ad35 packaging sequence or a functional fragment thereof. The present disclosure includes, among other things, HDAd35 donor vectors and genomes in which E1 or a fragment thereof is deleted (e.g., where the E1 deletion includes deletion of nucleotides 481-3112 of GenBank Accession No. AX049983 or corresponding positions of another Ad35 vector sequence provided herein). The present disclosure includes, among other things, HDAd35 vectors and genomes in which E3 or a fragment thereof is deleted (e.g., where the E3 deletion includes deletion of nucleotides 27609 to 30402 or 27435-30542 of GenBank Accession No. AX049983 or corresponding positions of another Ad35 vector sequence provided herein).

The present disclosure includes, among other things, Ad35 helper vectors and genomes that include two recombination site elements that flank a packaging sequence or functionally contributing (e.g., functionally required) fragment thereof, each recombination site element including a recombination site, where the two recombination sites are sites for the same recombinase. Construction of an Ad35 helper vector, as noted above, cannot be predictably engineered from existing knowledge relating to other vectors. To the contrary, relevant sequences of Ad35 are very different from, e.g., corresponding sequences of Ad5 (compare, e.g., the 5′ 600 to 620 nucleotides of Ad35 and Ad5). Moreover, packaging sequence are serotype-specific. The Ad35 packaging sequence includes sequences that correspond to at least Ad5 packaging single sequences AI, AII, AIII, AIV, and AV. Accordingly, production of an Ad35 helper vector requires several unpredictable determinations, including (1) identification of the Ad35 packaging sequence or functionally contributing (e.g., functionally required) fragment thereof to be flanked by recombinase sites (e.g., loxP sites) by insertion of recombinase site elements into the subject genome, which is not straightforward where sequence similarity is limited; (2) identification of recombinase site element insertions that do not negate propagation of the helper vector (under conditions where the packaging sequence or functionally contributing (e.g., functionally required) fragment thereof is not excised), which cannot be predicted; and/or (3) identification of spacing between the recombination site elements that permits efficient deletion of the packaging sequence or functionally contributing (e.g., functionally required) fragment thereof while reducing helper virus packaging during production of HDAd35 donor vectors (e.g., in a cre recombinase-expressing cell line such as the 116 cell line).

The present disclosure includes a plurality of exemplary Ad35 helper vectors and genomes that (1) include loxP sites flanking a functionally contributing or functionally required fragment of the Ad35 packaging sequence, at least in that recombination of the loxP sites causing excision of the flanked sequence reduces propagation of the vector by, e.g., at least 20%, 30%, 40%, 50%, 60%, 70%, 80%, 90%, 91%, 92%, 93%, 94%, 95%, 96%, 97%, 98%, 99%, or 100% (e.g., reduces propagation of the vector by a percentage having a lower bound of 20%, 30%, 40%, 50%, 60%, 70%, and an upper bound of 60%, 70%, 80%, 90%, 91%, 92%, 93%, 94%, 95%, 96%, 97%, 98%, 99%, or 100%), optionally where percent propagation is measured as the number of viral particles produced by propagation of excised vector (recombinase site-flanked sequence excised) as compared to complete vector (recombinase site-flanked sequence not excised) or wild-type Ad35 vector under comparable conditions.

In at least one exemplary Ad35 helper vector, a recombinase site element (e.g., a loxP element) is inserted after nucleotide 178 and a recombinase site element (e.g., a loxP element) is inserted after nucleotide 437. Excision of the loxP-flanked sequence removes packaging sequence sequences A1 to AIV. In certain such embodiments, deletion of nucleotides 345-3113 removes the E1 gene as well as packaging single sequences AVI and AVII. Accordingly, the flanked packaging sequence or fragment thereof corresponds to positions 179-344. Vectors according to this description were shown to propagate.

In at least one exemplary Ad35 helper vector, a recombinase site element (e.g., a loxP element) is inserted after nucleotide 178 and a recombinase site element (e.g., a loxP element) is inserted after nucleotide 481, where nucleotides 179-365 are deleted (removing packaging sequence sequences A1 to AV, such that remaining sequences AVI and AVII are in the nucleic acid sequence flanked by the recombinase site elements. In certain such embodiments, deletion of nucleotides 482-3113 removes the E1 gene. Accordingly, the flanked packaging sequence or fragment thereof corresponds to positions 366-481. Vectors according to this description were shown to propagate.

In at least one exemplary Ad35 helper vector, a recombinase site element (e.g., a loxP element) is inserted after nucleotide 154 and a recombinase site element (e.g., a loxP element) is inserted after nucleotide 481, In certain such embodiments, deletion of nucleotides 482-3113 removes the E1 gene. Accordingly, the flanked packaging sequence or fragment thereof corresponds to positions 155-481. Vectors according to this description were shown to propagate.

In at least one exemplary Ad35 helper vector, a recombinase site element (e.g., a loxP element) is inserted after nucleotide 158 and a recombinase site element (e.g., a loxP element) is inserted after nucleotide 480. Vectors according to this description were shown to propagate. In certain such embodiments, nucleotides 27388-30402 including E3 region are deleted. In certain embodiments, the vector is an Ad35⁺⁺ vector.

In at least one exemplary Ad35 helper vector, a recombinase site element (e.g., a loxP element) is inserted after nucleotide 158 and a recombinase site element (e.g., a loxP element) is inserted after nucleotide 446. Vectors according to this description were shown to propagate. In certain such embodiments, nucleotides 27388-30402 including E3 region are deleted. In certain embodiments, the vector is an Ad35⁺⁺ vector.

In at least one exemplary Ad35 helper vector, a recombinase site element (e.g., a loxP element) is inserted after nucleotide 179 and a recombinase site element (e.g., a loxP element) is inserted after nucleotide 480. Vectors according to this description were shown to propagate. In certain such embodiments, nucleotides 27388-30402 including E3 region are deleted. In certain embodiments, the vector is an Ad35⁺⁺ vector.

In at least one exemplary Ad35 helper vector, a recombinase site element (e.g., a loxP element) is inserted after nucleotide 206 and a recombinase site element (e.g., a loxP element) is inserted after nucleotide 480. Vectors according to this description were shown to propagate. In certain such embodiments, nucleotides 27,388-30,402 including E3 region are deleted. In certain embodiments, nucleotides 27,607-30,409 or 27,609-30,402 are deleted. In certain embodiments, nucleotides 27,240-27,608 are not deleted.

In at least one exemplary Ad35 helper vector, a recombinase site element (e.g., a loxP element) is inserted after nucleotide 139 and a recombinase site element (e.g., a loxP element) is inserted after nucleotide 446. In certain such embodiments, nucleotides 27609-30402 are deleted.

In at least one exemplary Ad35 helper vector, a recombinase site element (e.g., a loxP element) is inserted after nucleotide 158 and a recombinase site element (e.g., a loxP element) is inserted after nucleotide 446. In certain such embodiments, nucleotides 27609-30402 are deleted.

In at least one exemplary Ad35 helper vector, a recombinase site element (e.g., a loxP element) is inserted after nucleotide 179 and a recombinase site element (e.g., a loxP element) is inserted after nucleotide 446. In certain such embodiments, nucleotides 27609-30402 are deleted.

In at least one exemplary Ad35 helper vector, a recombinase site element (e.g., a loxP element) is inserted after nucleotide 201 and a recombinase site element (e.g., a loxP element) is inserted after nucleotide 446. In certain such embodiments, nucleotides 27609-30402 are deleted.

In at least one exemplary Ad35 helper vector, a recombinase site element (e.g., a loxP element) is inserted after nucleotide 158 and a recombinase site element (e.g., a loxP element) is inserted after nucleotide 481. In certain such embodiments, nucleotides 27609-30402 are deleted.

In at least one exemplary Ad35 helper vector, a recombinase site element (e.g., a loxP element) is inserted after nucleotide 179 and a recombinase site element (e.g., a loxP element) is inserted after nucleotide 384. In certain such embodiments, nucleotides 27609-30402 are deleted.

In at least one exemplary Ad35 helper vector, a recombinase site element (e.g., a loxP element) is inserted after nucleotide 179 and a recombinase site element (e.g., a loxP element) is inserted after nucleotide 481. In certain such embodiments, nucleotides 27609-30402 are deleted.

In at least one exemplary Ad35 helper vector, a recombinase site element (e.g., a loxP element) is inserted after nucleotide 206 and a recombinase site element (e.g., a loxP element) is inserted after nucleotide 481. In certain such embodiments, nucleotides 27609-30402 are deleted.

In various embodiments, at least portion of an Ad35 packaging sequence flanked by recombinase DRs corresponds to: nucleotides 179-344; nucleotides 366-481; nucleotides 155-481; nucleotides 159-480; nucleotides 159-446; nucleotides 180-480; nucleotides 207-480; nucleotides 140-446; nucleotides 159-446; nucleotides 180-446; nucleotides 202-446; nucleotides 159-481; nucleotides 180-384; nucleotides 180-481; or nucleotides 207-481 of the Ad35 sequence according to GenBank Accession No. AX049983. In various embodiments, at least a portion of an Ad35 packaging sequence flanked by recombinase DRs corresponds to nucleotides 138-481 of the Ad35 sequence according to GenBank Accession No. AX049983. In various embodiments, an Ad35 genome includes recombinase direct repeats (DRs) within 550 nucleotides of the 5′ end of the Ad35 genome that functionally disrupt the Ad35 packaging signal but not the 5′ Ad35 inverted terminal repeat (ITR). In various embodiments, recombinase DRs are LoxP sites. In various embodiments, recombinase DRs are rox, vox, AttB, or AttP sites. In various embodiments, an Ad35 helper genome includes Ad5 E4orf6 for amplification in 293 T cells.

An additional optional engineering consideration can be engineering of a helper genome having a size that permits separation of helper vector from HDAd35 donor vector by centrifugation, e.g., by CsCl ultracentrifugation. One means of achieving this result is to increase the size of the helper genome as compared to a typical Ad35 genome, which has a wild-type length of 34,794 bp. In particular, adenoviral genomes can be increased by engineering to at least 104% of wild-type length. Certain helper vectors of the present disclosure include the Ad35 E1 region and E4 region, delete the E3 region, and can accommodate a payload and/or stutter sequence.

Ad35 helper vectors can be used for production of Ad35 donor vectors. Production of HDAd35++ vectors can include co-transfection of a plasmid containing the HDAd vector genome and a packaging-defective helper virus that provides structural and non-structural viral proteins. The helper virus genome can rescue propagation of the Ad35 donor vector and Ad35 donor vector can be produced, e.g., at a large scale, and isolated. Various protocols are known in the art, e.g., at Palmer et al., 2009 Gene Therapy Protocols. Methods in Molecular Biology, Volume 433. Humana Press; Totowa, N.J.: 2009. pp. 33-53.

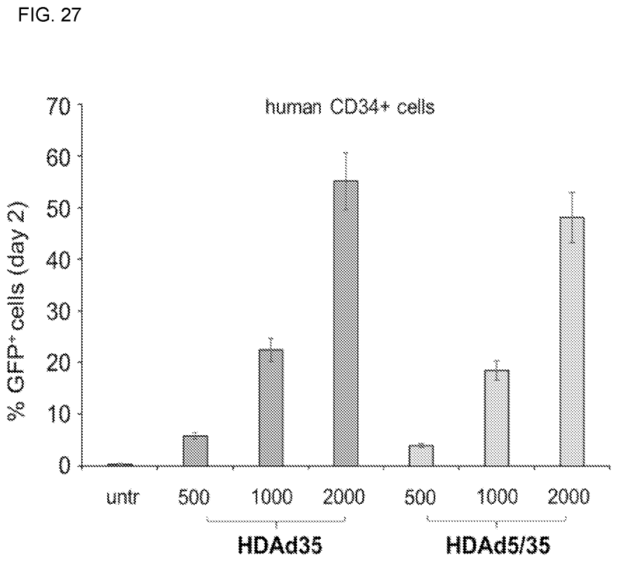

The present disclosure includes exemplary data demonstrating that HDAd35 donor vectors of the present disclosure perform comparably to HDAd5/35 donor vectors in transduction of human CD34+ cells, as measured by percent of contacted cells expressing a payload coding sequence encoding GFP. Results were confirmed at multiple MOIs ranging from 500 to 2000 vector particles per contacted cell. Exemplary experiments were conducted using HDAd35 donor vectors were produced using an Ad35 helper vector as disclosed above, where loxP sites flanked nucleotides 366-481 (see, e.g., FIG. 27).

Various exemplary donor vectors are provided herein. The present disclosure provides, as non-limiting examples, HDAd35 donor genomes as set forth in Tables 4-7.

TABLE 4
Exemplary HDAd35 donor vector according to SEQ ID NO: 189.

| | Position in |
|---|---|
| Sequence Feature | SEQ ID NO: 189 |
| Ad35 5′ (including ITR, Packaging Sequence) | Start: 1 End: 481 |
| FRT recombinase direct repeat | Start: 14126 End: 14159 |
| | (Complementary) |
| pT4 transposase inverted repeat | Start: 14220 End: 14463 |
| EF1α promoter | Start: 14491 End: 15825 |
| mgmt^{P140K} selection cassette | Start: 15843 End: 16466 |
| polyA sequence | Start: 16484 End: 16705 |
| pT4 transposase inverted repeat | Start: 16735 End: 17000 |
| FRT recombinase direct repeat | Start: 17107 End: 17140 |
| | (Complementary) |
| Ad35 3′ (including ITR) | Start: 28823 End: 29230 |

TABLE 5
Exemplary HDAd35 donor vector according to SEQ ID NO: 190

| | Position in SEQ |
|---|---|
| Sequence Feature | ID NO: 190 |
| Ad35 5′ (including ITR, Packaging Sequence) | Start: 1 End: 481 |
| FRT recombinase direct repeat | Start: 14126 End: 14159 |
| | (Complementary) |
| pT4 transposase inverted repeat | Start: 14220 End: 14463 |
| EF1α promoter | Start: 14478 End: 15812 |
| mgmt^{P140K} selection cassette | Start: 15830 End: 16450 |
| 2A peptide-encoding sequence | Start: 16451 End: 16522 |
| GFP-encoding sequence | Start: 16523 End: 17242 |
| SV40 polyA sequence | Start: 17269 End: 17390 |
| pT4 transposase inverted repeat | Start: 17501 End: 17766 |
| FRT recombinase direct repeat | Start: 17873 End: 17906 |
| | (Complementary) |
| Ad35 3′ (including ITR) | Start: 29589 End: 29996 |

TABLE 6
Exemplary HDAd35 donor vector according to SEQ ID NO: 191.

| | Position in |
|---|---|
| Sequence Feature | SEQ ID NO: 191 |
| Ad35 5′ (including ITR, Packaging Sequence) | Start: 1 End: 481 |
| FRT recombinase direct repeat | Start: 14126 End: 14159 |
| | (Complementary) |
| pT4 transposase inverted repeat | Start: 14220 End: 14463 |
| EF1α promoter | Start: 14478 End: 15812 |
| mgmt^{P140K} selection cassette | Start: 15830 End: 16450 |
| 2A peptide-encoding sequence | Start: 16451 End: 16522 |
| mCherry-encoding sequence | Start: 16526 End: 17230 |
| SV40 polyA sequence | Start: 17259 End: 17380 |
| pT4 transposase inverted repeat | Start: 17491 End: 17756 |
| FRT recombinase direct repeat | Start: 17863 End: 17896 |
| | (Complementary) |
| Ad35 3′ (including ITR) | Start: 29579 End: 29986 |

TABLE 7
Exemplary support vector according to SEQ ID NO: 192.

| | Position in |
|---|---|
| Sequence Feature | SEQ ID NO: 192 |
| Ad35 5′ (including ITR, Packaging Sequence) | Start: 1 End: 481 |
| PGK promoter | Start: 14103 End: 14614 |
| SB100x transposase-encoding sequence | Start: 14763 End: 15785 |
| BGH polyA sequence | Start: 15811 End: 16128 |
| B-globin polyA sequence | Start: 16088 End: 16376 |
| | (Complementary) |
| Flpe recombinase-encoding sequence | Start: 16488 End: 17759 |
| | (Complementary) |
| EF1α promoter | Start: 17780 End: 18895 |
| | (Complementary) |
| Ad35 3′ (including ITR) | Start: 29751 End: 30158 |

TABLE 8
Exemplary Ad35 helper vector according to SEQ ID NO: 180

| | Position in |
|---|---|
| Sequence Feature | SEQ ID NO: 180 |
| Ad35 5′ (including ITR)(Ad35 nt 1-178) | Start: 2582 End: 2759 |
| LoxP recombinase site | Start: 2768 End: 2801 |
| Ad35 packaging sequence (Ad35 nt 179-344) | Start: 2808 End: 2973 |
| LoxP recombinase site | Start: 2974 End: 3007 |
| Ad35 sequence (Ad35 nt 3112-27435) | Start: 3016 End: 27338 |
| Lambda-1 sequence | Start: 27393 End: 29862 |
| | (Complementary) |
| BGH polyA sequence | Start: 30176 End: 30390 |
| CopGFP-encoding sequence | Start: 30415 End: 31080 |
| | (Complementary) |
| CMV promoter | Start: 31127 End: 31779 |
| | (Complementary) |
| Lambda-2 sequence | Start: 31831 End: 33360 |
| Ad35 sequence (Ad35 nt 30544-31879) | Start: 33421 End: 34756 |
| Ad5 E4orf6 sequence | Start: 34752 End: 35866 |
| Ad35 3′ (including ITR) | Start: 35864 End: 37686 |
| (Ad35 nt 32972-34794) | |

TABLE 9
Exemplary Ad35 helper vector according to SEQ ID NO: 172.

| | Position in |
|---|---|
| Sequence Feature | SEQ ID NO: 172 |
| Ad35 5′ (including ITR) (Ad35 nt 1-178) | Start: 2582 End: 2759 |
| LoxP recombinase site | Start: 2768 End: 2801 |
| Ad35 packaging sequence (Ad35 nt 366-481) | Start: 2808 End: 2923 |
| LoxP recombinase site | Start: 2924 End: 2957 |
| Ad35 sequence (Ad35 nt 3112-2743) | Start: 2966 End: 27288 |
| Lambda-1 sequence | Start: 27343 End: 29812 |
| | (Complementary) |
| BGH polyA sequence | Start: 30126 End: 30340 |
| CopGFP-encoding sequence | Start: 30365 End: 31030 |
| | (Complementary) |
| CMV promoter | Start: 31077 End: 31729 |
| | (Complementary) |
| Lambda-2 sequence | Start: 31781 End: 33310 |
| Ad35 sequence (Ad35 nt 30544-31879) | Start: 33371 End: 34706 |
| Ad5 E4orf6 sequence | Start: 34702 End: 35816 |
| Ad35 3′ (including ITR) | Start: 35814 End: 37636 |
| (Ad35 nt 32972-34794) | |

TABLE 10
Exemplary Ad35 helper vector according to SEQ ID NO: 173.

| | Position in |
|---|---|
| Sequence Feature | SEQ ID NO: 173 |
| Ad35 5′ (including ITR) (Ad35 nt 1-154) | Start: 2582 End: 2735 |
| LoxP recombinase site | Start: 2744 End: 2777 |
| Ad35 packaging sequence (Ad35 nt 155-481) | Start: 2784 End: 3110 |
| LoxP recombinase site | Start: 3111 End: 3144 |
| Ad35 sequence (Ad35 nt 3112-27435) | Start: 3153 End: 27475 |
| Lambda-1 sequence | Start: 27530 End: 29999 |
| | (Complementary) |
| BGH polyA sequence | Start: 30313 End: 30527 |
| CopGFP-encoding sequence | Start: 30552 End: 31217 |
| | (Complementary) |
| CMV promoter | Start: 31264 End: 31916 |
| | (Complementary) |
| Lambda-2 sequence | Start: 31968 End: 33497 |
| Ad35 sequence (Ad35 nt 30544-31879) | Start: 33558 End: 34893 |
| Ad5 E4orf6 sequence | Start: 34889 End: 36003 |
| Ad35 3′ (including ITR) | Start: 36001 End: 37823 |
| (Ad35 nt 32972-34794) | |

This present disclosure includes production and use of Ad35 vectors and demonstration of efficacy for transduction of CD34+ cells. Three exemplary Ad35 vectors were produced, with different structures (including different LoxP placement).

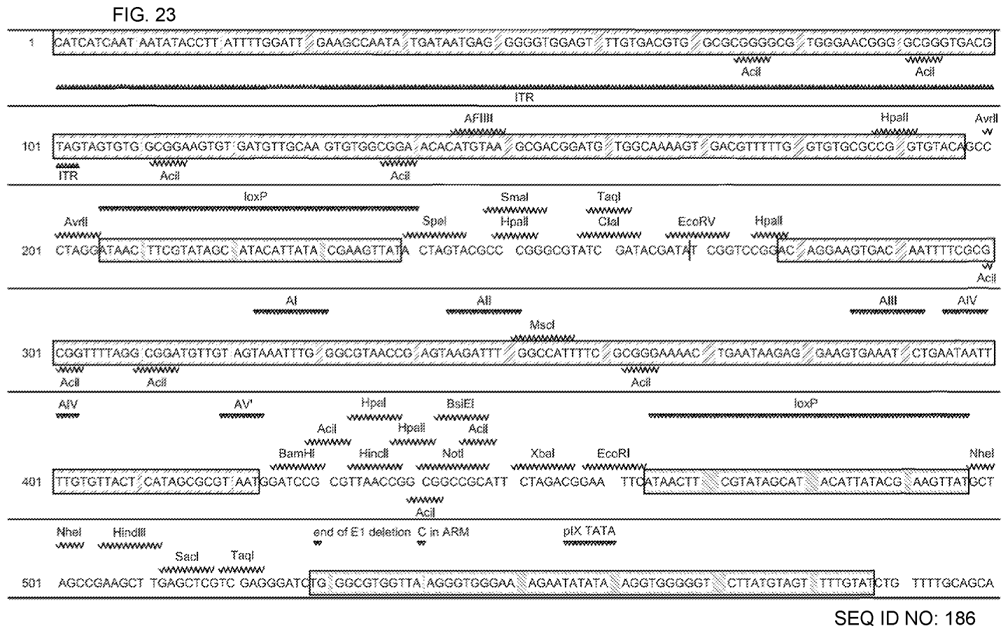

The left end of a representative Ad5/35 helper virus genome is shown in FIG. 23 (SEQ ID NO: 186). The sequences shaded in dark grey correspond to the native Ad5 sequence, i.e., the unshaded or light grey highlighted sequences were artificially introduced. The sequences highlighted in light grey are two copies of the (tandemly repeated) loxP sequences. In the presence of “cre recombinase” protein, the nucleotide sequence between the two loxP sequences are deleted (leaving behind one copy of loxP). Because the Ad5 sequence between the loxP sites is essential for packaging the adenoviral DNA into capsids (in the nucleus of the producer cell), this deletion renders the helper adenovirus genome DNA deficient for packaging. Consequently, the efficiency of the deletion process has a direct influence of the level of packaged helper genomic DNA (the undesired helper virus “contamination”). In view of the above, in order to translate the same scheme to adenovirus serotypes other than Ad5, it is desirable to achieve the following: 1. Identify the sequences that are essential for packaging, so that they can be flanked by loxP sequence insertions and deleted in the presence of cre recombinase. Identification of these sequences is not straightforward if there is little similarity in sequences. 2. Determine where in the native DNA sequence the insertion of loxP sequence would have the least effect for the propagation and packaging of helper virus (in the absence of cre recombinase). 3. Determine the spacing between the loxP sequences to allow for efficient deletion of packaging sequences and keeping helper virus packaging to a minimum during the production of helper-dependent adenovirus (i.e., in a cre recombinase—expressing cell line such as the 116 cell line).

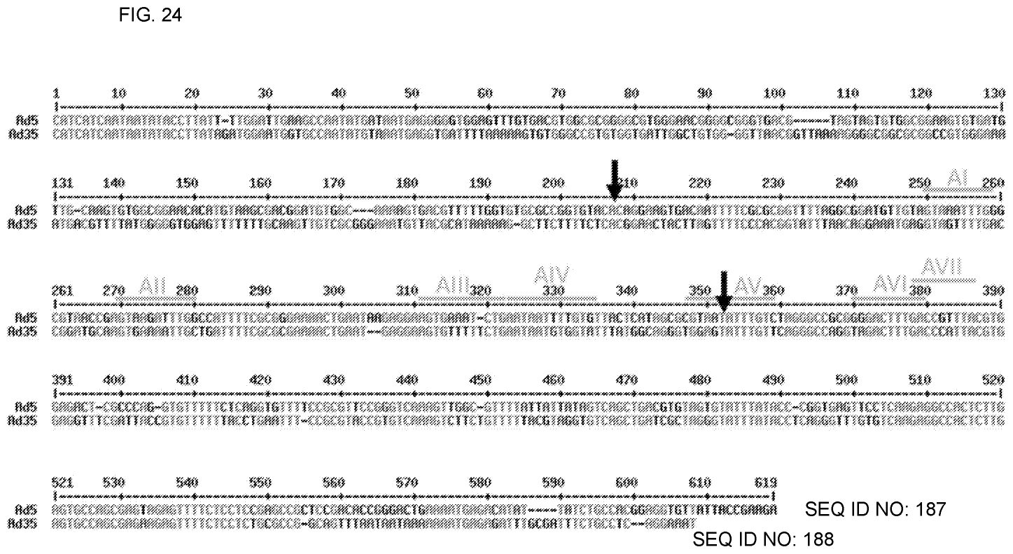

FIG. 24 shows an alignment of representative Ad5 and Ad35 packaging signals (SEQ ID NOs: 187 and 188). The alignment of the left end sequences of Ad5 with Ad35 help in identifying packaging signals. Motifs in the Ad5 sequence that are important for packaging (AI through AV) are indicated with lines (see also FIG. 1B of Schmid et al., J Virol., 71(5):3375-4, 1997). The location of exemplary loxP insertion sites are indicated by black arrows. These insertions flank AI to AIV and disrupt AV. The additional packaging signal AVI and AVII, as indicated in Schmid et al., have been deleted in the Ad5 helper virus as part of the E1 deletion of this vector.

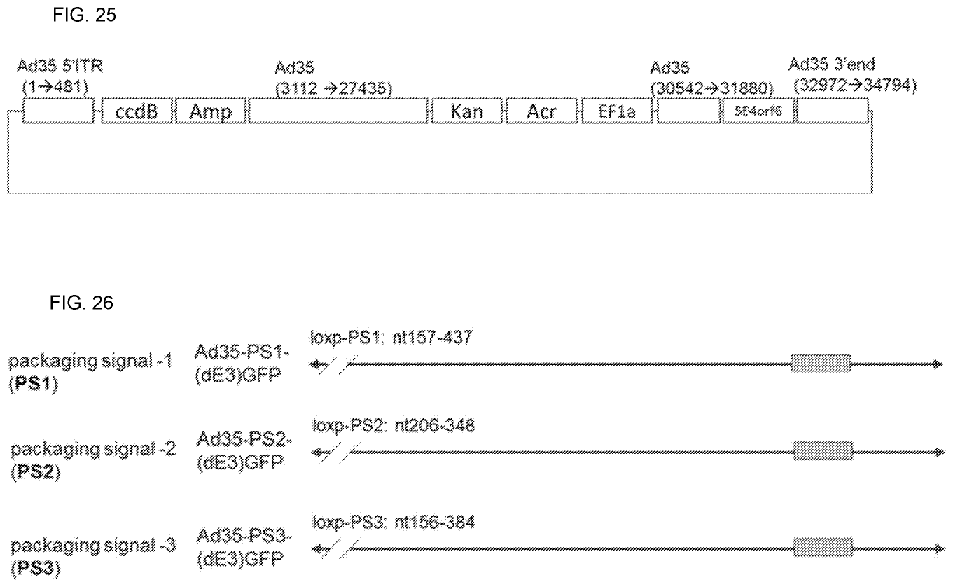

FIG. 25 is a schematic illustration of the Ad35 vector pAd35GLN-5E4. This is a first-generation (E1/E3-deleted) Ad35 vector derived from a vectorized Ad35 genome (Holden strain from the ATCC) using a recombineering technique (Zhang et al., Cell Rep. 19(8):1698-17-9, 2017). This vector plasmid was then used to insert loxP sites.

The packaging site (PS)1 LoxP insertion sites are after nucleotide 178 and 344; this Ad35 vector is exemplified in SEQ ID NO: 180. This LoxP placement is expected to remove AI to AIV. The rest of the packaging signal including AVI and AVII (after 344) has been deleted (as part of the E1 deletion at positions 345 to 3113). The PS2 LoxP insertion sites are after nucleotide 178 and 481; this Ad35 vector is exemplified in SEQ ID NO: 172. Additionally, nucleotides 179 to 365 have been deleted, so AI through AV are not present. The remaining packaging motifs AVI and AVII are removable by cre recombinase during HDAd production. The E1 deletion is from 482 to 3113. The PS3 LoxP insertion sites are after nucleotide 154 and 481; this Ad35 vector is exemplified in SEQ ID NO: 173. The packaging signal structure of these three vectors is provided in FIG. 26.

Three engineered vectors could be rescued. The percentage of viral genomes with rearranged loxP sites was 50, 20, and 60% for PS1, PS2, and PS3, respectively. Rearrangements occur when the lox P sites critically affected viral replication and gene expression.

This HDAd35 platform compared to a current HDAd5/35 platform is illustrated in FIG. 27. Both vectors contain a CMV-GFP cassette. The Ad35 vector does not contain immunogenic Ad5 capsid protein. These two vectors showed comparable transduction efficiency of CD34+ cells in vitro. Bridging study shows comparable transduction efficiency of CD34+ cells in vitro. Human HSCs, peripheral CD34+ cells from G-CSF mobilized donors were transduced with HDAd35 (produced with Ad35 helper P-2) or a chimeric vector containing the Ad5 capsid with fiber from Ad35, at MOIs 500, 1000, 2000 vp/cell. The percentage of GFP-positive cells was measured 48 hours after adding the virus in three independent experiments.

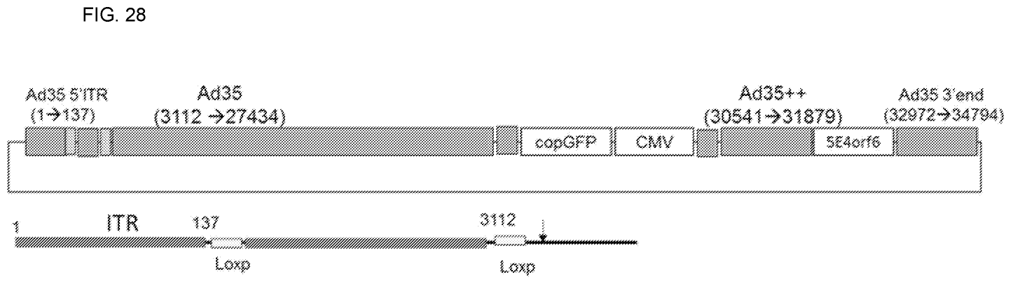

The PS2 helper vector was remade (as illustrated in FIG. 28) for use in monkey studies. The following actions were taken to make this version: deletion of E1 region, a mutant packaging signal flanked by Loxp, mutant packaging sequence, deletion of E3 region (27435→30540), replace with Ad5E4orf6, insertion of stutter DNA flanking copGFP cassette, and introduction of mutation in the knob to make Ad35K++.

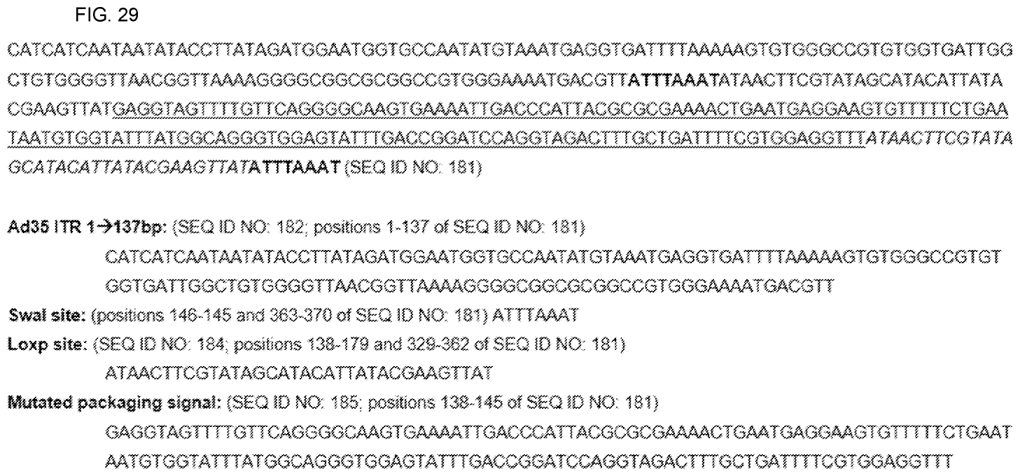

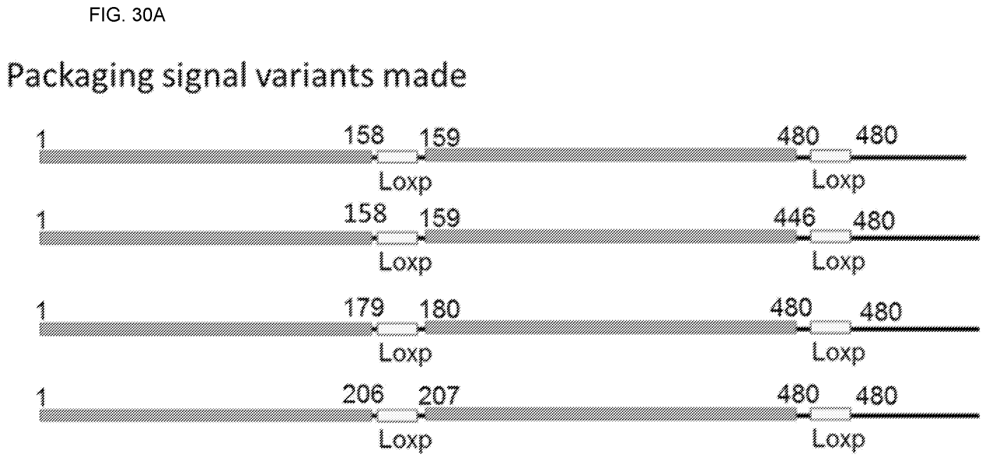

FIG. 29 shows a mutated packaging signal sequence. Residues 1 through 137 are the Ad35 ITR. Text in bold are SwaI sites, the Loxp site is italicized, and the mutated packaging signal is underlined. For clarity, these sequences are shown individually in FIG. 29.

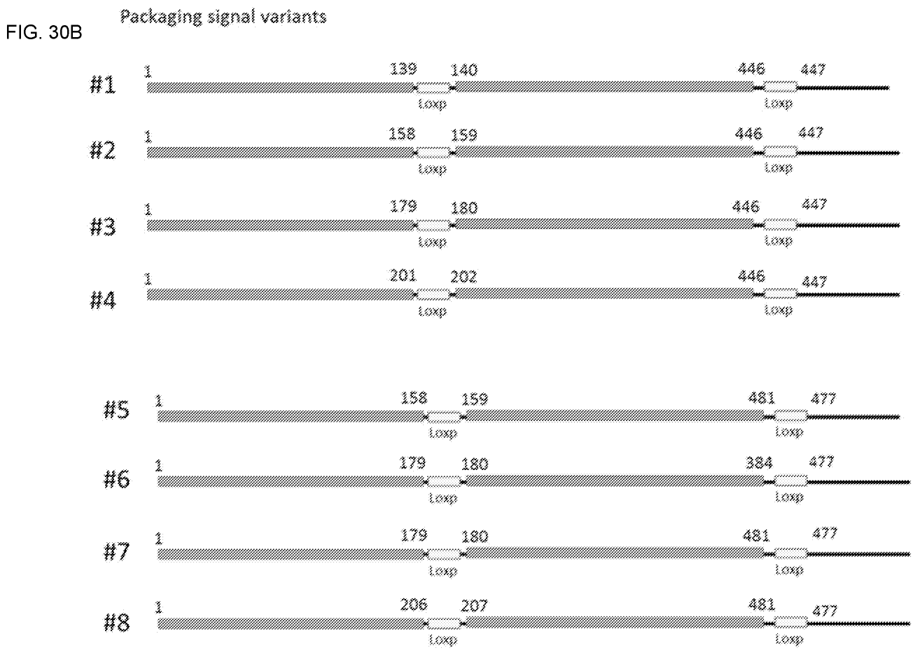

Four Ad35 helper vector packaging signal variants were made (FIG. 30A). The E3 region (27388→30402) was deleted and the CMV-eGFP cassette was located within an E3 deletion, Ad35K++, and eGFP was used instead of copGFP. The LoxP sites in these four packaging signal variants are at the illustrated positions (FIG. 120A). All four helper vectors could be rescued.

FIG. 30B is a schematic representation of eight additional packaging signal variants, with the specified the LoxP sites.

In certain additional helper vector and packaging signal variants, changes were made to the helper vector in FIG. 30A, such as shortening the E3 deletion (27609→30402). Further description of adenoviral vectors is found in related application No. PCT/US2020/040756, which is incorporated herein by reference in its entirety and with respect to adenoviral vectors, in particular with respect to Ad35 vectors, including HDAd35 vectors and related vectors.

Vectors described herein can be administered in coordination with HSC mobilization factors. In certain embodiments, adenoviral vector formulations described herein can be administered in concert with HSC mobilization. In particular embodiments, administration of viral vector occurs concurrently with administration of one or more mobilization factors (also referred to herein in the alternative as mobilization agents). In particular embodiments, administration of viral vector follows administration of one or more mobilization factors. In particular embodiments, administration of viral vector follows administration of a first one or more mobilization factors and occurs concurrently with administration of a second one or more mobilization factors.

Mobilizing agents include cytotoxic drugs, cytokines, and/or small molecules. Agents for HSPC mobilization include, for example, granulocyte-colony stimulating factor (G-CSF), granulocyte macrophage colony stimulating factor (GM-CSF), plerixafor, SCF, S-CSF, a CXCR4 antagonist, a CXCR2 agonist, and Gro-Beta (GRO-ρ). In various embodiments, a CXCR4 antagonist is AMD3100 and/or a CXCR2 agonist is GRO-β. In particular embodiments, a mobilizing agent is C4, a CXC chemokine ligand for the CXCR2 receptor.

Plerixafor is a bicyclam molecule that specifically and reversibly blocks SDF-1 binding to CXCR4. Plerixafor is also known commercially under the trade names Mozobil, Revixil, UMK121, AMD3000, AMD3100, GZ316455, JM3100, and SDZSID791. In certain embodiments, plerixafor is used as a single agent for mobilization of HSPCs.

Gro-Beta rapidly mobilizes short- and long-term repopulating cells in mice and/or monkeys and synergistically enhances mobilization responses with G-CSF (Pelus and Fukuda, Exp. Hematol. 34(8):1010-1020, 2006). Furthermore, Gro-Beta can be combined with antagonists of VLA4 to synergistically increase circulating HSPC numbers (Karpova et al., Blood. 129(21):2939-2949, 2017). In various embodiments, the present disclosure includes a Gro-Beta agent as disclosed in WO 2019/089833 (e.g., Gro-Beta, Gro-BetaT, and a variant thereof), WO 2019/113375, and/or WO 2019/136159, each of which is incorporated herein by reference in its entirety and in particular with respect to sequences relating to Gro-Beta and modified forms thereof. In various embodiments, the present disclosure includes a Gro-Beta agent that is MGTA 145 (Magenta Therapeutics). In various embodiments, the present disclosure includes a Gro-Beta agent form that does not include amino acids corresponding to the four N-terminal amino acids of canonical Gro-Beta.

G-CSF is a cytokine whose functions in HSPC mobilization can include the promotion of granulocyte expansion and both protease-dependent and independent attenuation of adhesion molecules and disruption of the SDF-1/CXCR4 axis. In particular embodiments, any commercially available form of G-CSF known to one of ordinary skill in the art can be used in the methods and formulations as disclosed herein, for example, Filgrastim (Neupogen®, Amgen Inc., Thousand Oaks, Calif.) and PEGylated Filgrastim (Pegfilgrastim, NEULASTA®, Amgen Inc., Thousand Oaks, Calif.).

GM-CSF is a monomeric glycoprotein also known as colony-stimulating factor 2 (CSF2) that functions as a cytokine and is naturally secreted by macrophages, T cells, mast cells, natural killer cells, endothelial cells, and fibroblasts. In particular embodiments, any commercially available form of GM-CSF known to one of ordinary skill in the art can be used in the methods and formulations as disclosed herein, for example, Sargramostim (Leukine, Bayer Healthcare Pharmaceuticals, Seattle, Wash.) and molgramostim (Schering-Plough, Kenilworth, N.J.).

AMD3100 (MOZOBIL™, PLERIXAFOR™; Sanofi-Aventis, Paris, France), a synthetic organic molecule of the bicyclam class, is a chemokine receptor antagonist and reversibly inhibits SDF-1 binding to CXCR4, promoting HSPC mobilization. AMD3100 is approved to be used in combination with G-CSF for HSPC mobilization in patients with myeloma and lymphoma. The structure of AMD3100 is:

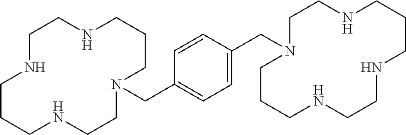 [SMILES: c1cc(CN2CCCNCCNCCCNCC2)ccc1CN1CCCNCCNCCCNCC1]

SCF, also known as KIT ligand, KL, or steel factor, is a cytokine that binds to the c-kit receptor (CD117). SCF can exist both as a transmembrane protein and a soluble protein. This cytokine plays an important role in hematopoiesis, spermatogenesis, and melanogenesis. In particular embodiments, any commercially available form of SCF known to one of ordinary skill in the art can be used in the methods and formulations as disclosed herein, for example, recombinant human SCF (Ancestim, STEMGEN®, Amgen Inc., Thousand Oaks, Calif.).

Chemotherapy used in intensive myelosuppressive treatments also mobilizes HSPCs to the peripheral blood as a result of compensatory neutrophil production following chemotherapy-induced aplasia. In particular embodiments, chemotherapeutic agents that can be used for mobilization of HSPCs include cyclophosphamide, etoposide, ifosfamide, cisplatin, and cytarabine.

Additional agents that can be used for cell mobilization include: CXCL12/CXCR4 modulators (e.g., CXCR4 antagonists: POL6326 (Polyphor, Allschwil, Switzerland), a synthetic cyclic peptide which reversibly inhibits CXCR4; BKT-140 (4F-benzoyl-TN14003; Biokine Therapeutics, Rehovit, Israel); TG-0054 (Taigen Biotechnology, Taipei, Taiwan); CXCL12 neutralizer NOX-A12 (NOXXON Pharma, Berlin, Germany) which binds to SDF-1, inhibiting its binding to CXCR4); Sphingosine-1-phosphate (S1 P) agonists (e.g., SEW2871, Juarez et al. Blood 119: 707-716, 2012); vascular cell adhesion molecule-1 (VCAM) or very late antigen 4 (VLA-4) inhibitors (e.g., Natalizumab, a recombinant humanized monoclonal antibody against a4 subunit of VLA-4 (Zohren et al. Blood 111: 3893-3895, 2008); B105192, a small molecule inhibitor of VLA-4 (Ramirez et al. Blood 114: 1340-1343, 2009)); parathyroid hormone (Brunner et al. Exp Hematol. 36: 1157-1166, 2008); proteasome inhibitors (e.g., Bortezomib, Ghobadi et al. ASH Annual Meeting Abstracts. p. 583, 2012); Groβ, a member of CXC chemokine family which stimulates chemotaxis and activation of neutrophils by binding to the CXCR2 receptor (e.g., SB-251353, King et al. Blood 97: 1534-1542, 2001); stabilization of hypoxia inducible factor (HIF) (e.g., FG-4497, Forristal et al. ASH Annual Meeting Abstracts. p. 216, 2012); Firategrast, an α4β1 and α4β7 integrin inhibitor (α4β1/7) (Kim et al. Blood 128: 2457-2461, 2016); Vedolizumab, a humanized monoclonal antibody against the a487 integrin (Rosario et al. Clin Drug Investig 36: 913-923, 2016); and BOP (N-(benzenesulfonyl)-L-prolyl-L-O-(1-pyrrolidinylcarbonyl) tyrosine) which targets integrins α9β1/α4β1 (Cao et al. Nat Commun 7: 11007, 2016). Additional agents that can be used for HSPC mobilization are described in, for example, Richter R et al. Transfus Med Hemother 44:151-164, 2017, Bendall & Bradstock, Cytokine & Growth Factor Reviews 25: 355-367, 2014, WO 2003043651, WO 2005017160, WO 2011069336, U.S. Pat. Nos. 5,637,323, 7,288,521, 9,782,429, US 2002/0142462, and US 2010/02268.

In various embodiments, a mobilization regimen includes two or more mobilization agents. A historically used mobilization regimen includes a combination of cyclophosphamide (Cy) plus granulocyte-colony stimulating factor (G-CSF) (Bonig et al., Stem Cells. 27(4):836-837, 2009). Additional mobilizing agent regimens can include alpha4-integrin blockade with anti-functional antibodies and CXCR4 blockade with the small-molecule inhibitor plerixafor. Another mobilization regimen includes the combined regimen of GM-CSF or G-CSF with plerixafor.

In particular embodiments, a therapeutically effective amount of G-CSF includes 0.1 μg/kg to 100 μg/kg. In particular embodiments, a therapeutically effective amount of G-CSF includes 0.5 μg/kg to 50 μg/kg. In particular embodiments, a therapeutically effective amount of G-CSF includes 0.5 μg/kg, 1 μg/kg, 2 μg/kg, 3 μg/kg, 4 μg/kg, 5 μg/kg, 6 μg/kg, 7 μg/kg, 8 μg/kg, 9 μg/kg, 10 μg/kg, 11 μg/kg, 12 μg/kg, 13 μg/kg, 14 μg/kg, 15 μg/kg, 16 μg/kg, 17 μg/kg, 18 μg/kg, 19 μg/kg, 20 μg/kg, or more. In particular embodiments, a therapeutically effective amount of G-CSF includes 5 μg/kg. In particular embodiments, G-CSF can be administered subcutaneously or intravenously. In particular embodiments, G-CSF can be administered for 1 day, 2 consecutive days, 3 consecutive days, 4 consecutive days, 5 consecutive days, or more. In particular embodiments, G-CSF can be administered for 4 consecutive days. In particular embodiments, G-CSF can be administered for 5 consecutive days. In particular embodiments, as a single agent, G-CSF can be used at a dose of 10 μg/kg subcutaneously daily, initiated 3, 4, 5, 6, 7, or 8 days before viral vector delivery. In particular embodiments, G-CSF can be administered as a single agent followed by concurrent administration with another mobilization factor. In particular embodiments, G-CSF can be administered as a single agent followed by concurrent administration with AMD3100. In particular embodiments, a treatment protocol includes a 5 day treatment where G-CSF can be administered on day 1, day 2, day 3, and day 4 and on day 5, G-CSF and AMD3100 are administered 6 to 8 hours prior to viral vector administration.

Therapeutically effective amounts of GM-CSF to administer can include doses ranging from, for example, 0.1 to 50 μg/kg or from 0.5 to 30 μg/kg. In particular embodiments, a dose at which GM-CSF can be administered includes 0.5 μg/kg, 1 μg/kg, 2 μg/kg, 3 μg/kg, 4 μg/kg, 5 μg/kg, 6 μg/kg, 7 μg/kg, 8 μg/kg, 9 μg/kg, 10 μg/kg, 11 μg/kg, 12 μg/kg, 13 μg/kg, 14 μg/kg, 15 μg/kg, 16 μg/kg, 17 μg/kg, 18 μg/kg, 19 μg/kg, 20 μg/kg, or more. In particular embodiments, GM-CSF can be administered subcutaneously for 1 day, 2 consecutive days, 3 consecutive days, 4 consecutive days, 5 consecutive days, or more. In particular embodiments, GM-CSF can be administered subcutaneously or intravenously. In particular embodiments, GM-CSF can be administered at a dose of 10 μg/kg subcutaneously daily initiated 3, 4, 5, 6, 7, or 8 days before viral vector delivery. In particular embodiments, GM-CSF can be administered as a single agent followed by concurrent administration with another mobilization factor. In particular embodiments, GM-CSF can be administered as a single agent followed by concurrent administration with AMD3100. In particular embodiments, a treatment protocol includes a 5 day treatment where GM-CSF can be administered on day 1, day 2, day 3, and day 4 and on day 5, GM-CSF and AMD3100 are administered 6 to 8 hours prior to viral vector administration. A dosing regimen for Sargramostim can include 200 μg/m², 210 μg/m², 220 μg/m², 230 μg/m², 240 μg/m², 250 μg/m², 260 μg/m², 270 μg/m², 280 μg/m², 290 μg/m², 300 μg/m², or more. In particular embodiments, Sargramostim can be administered for 1 day, 2 consecutive days, 3 consecutive days, 4 consecutive days, 5 consecutive days, or more. In particular embodiments, Sargramostim can be administered subcutaneously or intravenously. In particular embodiments, a dosing regimen for Sargramostim can include 250 μg/m²/day intravenous or subcutaneous and can be continued until a targeted cell amount is reached in the peripheral blood or can be continued for 5 days. In particular embodiments, Sargramostim can be administered as a single agent followed by concurrent administration with another mobilization factor. In particular embodiments, Sargramostim can be administered as a single agent followed by concurrent administration with AMD3100. In particular embodiments, a treatment protocol includes a 5 day treatment where Sargramostim can be administered on day 1, day 2, day 3, and day 4 and on day 5, Sargramostim and AMD3100 are administered 6 to 8 hours prior to viral vector administration.

In particular embodiments, a therapeutically effective amount of AMD3100 includes 0.1 mg/kg to 100 mg/kg. In particular embodiments, a therapeutically effective amount of AMD3100 includes 0.5 mg/kg to 50 mg/kg. In particular embodiments, a therapeutically effective amount of AMD3100 includes 0.5 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, or more. In particular embodiments, a therapeutically effective amount of AMD3100 includes 4 mg/kg. In particular embodiments, a therapeutically effective amount of AMD3100 includes 5 mg/kg. In particular embodiments, a therapeutically effective amount of AMD3100 includes 10 μg/kg to 500 μg/kg or from 50 μg/kg to 400 μg/kg. In particular embodiments, a therapeutically effective amount of AMD3100 includes 100 μg/kg, 150 μg/kg, 200 μg/kg, 250 μg/kg, 300 μg/kg, 350 μg/kg, or more. In particular embodiments, AMD3100 can be administered subcutaneously or intravenously. In particular embodiments, AMD3100 can be administered subcutaneously at 160-240 μg/kg 6 to 11 hours prior to viral vector delivery. In particular embodiments, a therapeutically effective amount of AMD3100 can be administered concurrently with administration of another mobilization factor. In particular embodiments, a therapeutically effective amount of AMD3100 can be administered following administration of another mobilization factor. In particular embodiments, a therapeutically effective amount of AMD3100 can be administered following administration of G-CSF. In particular embodiments, a treatment protocol includes a 5-day treatment where G-CSF is administered on day 1, day 2, day 3, and day 4 and on day 5, G-CSF and AMD3100 are administered 6 to 8 hours prior to viral vector injection.

Therapeutically effective amounts of SCF to administer can include doses ranging from, for example, 0.1 to 100 μg/kg/day or from 0.5 to 50 μg/kg/day. In particular embodiments, a dose at which SCF can be administered includes 0.5 μg/kg/day, 1 μg/kg/day, 2 μg/kg/day, 3 μg/kg/day, 4 μg/kg/day, 5 μg/kg/day, 6 μg/kg/day, 7 μg/kg/day, 8 μg/kg/day, 9 μg/kg/day, 10 μg/kg/day, 11 μg/kg/day, 12 μg/kg/day, 13 μg/kg/day, 14 μg/kg/day, 15 μg/kg/day, 16 μg/kg/day, 17 μg/kg/day, 18 μg/kg/day, 19 μg/kg/day, 20 μg/kg/day, 21 μg/kg/day, 22 μg/kg/day, 23 μg/kg/day, 24 μg/kg/day, 25 μg/kg/day, 26 μg/kg/day, 27 μg/kg/day, 28 μg/kg/day, 29 μg/kg/day, 30 μg/kg/day, or more. In particular embodiments, SCF can be administered for 1 day, 2 consecutive days, 3 consecutive days, 4 consecutive days, 5 consecutive days, or more. In particular embodiments, SCF can be administered subcutaneously or intravenously. In particular embodiments, SCF can be injected subcutaneously at 20 μg/kg/day. In particular embodiments, SCF can be administered as a single agent followed by concurrent administration with another mobilization factor. In particular embodiments, SCF can be administered as a single agent followed by concurrent administration with AMD3100. In particular embodiments, a treatment protocol includes a 5 day treatment where SCF can be administered on day 1, day 2, day 3, and day 4 and on day 5, SCF and AMD3100 are administered 6 to 8 hours prior to viral vector administration.

In particular embodiments, growth factors GM-CSF and G-CSF can be administered to mobilize HSPC in the bone marrow niches to the peripheral circulating blood to increase the fraction of HSPCs circulating in the blood. In particular embodiments, mobilization can be achieved with administration of G-CSF/Filgrastim (Amgen) and/or AMD3100 (Sigma). In particular embodiments, mobilization can be achieved with administration of GM-CSF/Sargramostim (Amgen) and/or AMD3100 (Sigma). In particular embodiments, mobilization can be achieved with administration of SCF/Ancestim (Amgen) and/or AMD3100 (Sigma). In particular embodiments, administration of G-CSF/Filgrastim precedes administration of AMD3100. In particular embodiments, administration of G-CSF/Filgrastim occurs concurrently with administration of AMD3100. In particular embodiments, administration of G-CSF/Filgrastim precedes administration of AMD3100, followed by concurrent administration of G-CSF/Filgrastim and AMD3100. US 20140193376 describes mobilization protocols utilizing a CXCR4 antagonist with a 51 P receptor 1 (51 PR1) modulator agent. US 2011/0044997 describes mobilization protocols utilizing a CXCR4 antagonist with a vascular endothelial growth factor receptor (VEGFR) agonist.

In particular embodiments, an HSC enriching agent, such as a CD19 immunotoxin or 5-FU can be administered to enrich for HSPCs. CD19 immunotoxin can be used to deplete all CD19 lineage cells, which accounts for 30% of bone marrow cells. Depletion encourages exit from the bone marrow. By forcing HSPCs to proliferate (whether via CD19 immunotoxin of 5-FU, this stimulates their differentiation and exit from the bone marrow and increases transgene marking in peripheral blood cells.

Viral vectors can be administered concurrently with or following administration of one or more immunosuppression agents or immunosuppression regimens, which can include one or more steroids, IL-1 receptor antagonist, and/or an IL-6 receptor antagonist administration. These protocols can alleviate potential side effects of treatments.

IL-1 receptor antagonists are known and include ADC-1001 (Alligator Bioscience), FX-201 (Flexion Therapeutics), fusion proteins available from Bioasis Technologies, GQ-303 (Genequine Biotherapeutics GmbH), HL-2351 (Handok, Inc.), MBIL-1RA (ProteoThera, Inc.), Anakinra (Pivor Pharmaceuticals), human immunoglobin G or Globulin S (GC Pharma). IL-6 receptor antagonists are also known in the art and include tocilizumab, BCD-089 (Biocad), HS-628 (Zhejiang Hisun Pharm), and APX-007 (Apexigen).

In various embodiments, an immune suppression regimen is administered to a subject that also receives at least one viral gene therapy vector, where the immune suppression regimen includes administration of at least one immune suppression agent to the subject on (i) one or more days prior to administration to the subject of a first dose of the viral gene therapy vector; (ii) on the same day as administration of a first dose of the viral gene therapy vector; (iii) on the same day as administration of one or more second or other subsequent doses of the viral gene therapy vector; and/or (iv) on any of one or more, or all, days intervening between administration to the subject of the first dose of the viral gene therapy vector and administration of any of one or more, or all, second or other subsequent doses of the viral gene therapy vector.

In vitro gene therapy includes use of a vector, genome, or system of the present disclosure in a method of introducing exogenous DNA into a host cell (such as a target cell) and/or a nucleic acid (such as a target nucleic acid, such as a target genome), where the host cell or nucleic acid is not present in a multicellular organism (e.g., in a laboratory). In some embodiments, a target cell or nucleic acid is derived from a multicellular organism, such as a mammal (e.g., a mouse, rat, human, or non-human primate). In vitro engineering of a cell derived from a multicellular organism can be referred to as ex vivo engineering and can be used in ex vivo therapy. In various embodiments, methods and compositions of the present disclosure are utilized, e.g., as disclosed herein, to modify a target cell or nucleic acid derived from a first multicellular organism and the engineered target cell or nucleic acid is then administered to a second multicellular organism, such as a mammal (e.g., a mouse, rat, human, or non-human primate), e.g., in a method of adoptive cell therapy. In some instances, the first and second organisms are the same single subject organism. Return of in vitro engineered material to a subject from which the material was derived can be an autologous therapy. In some instances, the first and second organisms are different organisms (e.g., two organisms of the same species, e.g., two mice, two rats, two humans, or two non-human primates of the same species). Transfer of engineered material derived from a first subject to a second different subject can be an allogeneic therapy.

Ex vivo cell therapies can include isolation of stem, progenitor or differentiated cells from a patient or a normal donor, expansion of isolated cells ex vivo—with or without genetic engineering—and administration of the cells to a subject to establish a transient or stable graft of the infused cells and/or their progeny. Such ex vivo approaches can be used, for example, to treat an inherited, infectious or neoplastic disease, to regenerate a tissue or to deliver a therapeutic agent to a disease site. In various ex vivo therapies there is no direct exposure of the subject to the gene transfer vector, and the target cells of transduction can be selected, expanded and/or differentiated, before or after any genetic engineering, to improve efficacy and safety.

Ex vivo therapies include hematopoietic stem cell (HSC) transplantation (HCT). Autologous HSC gene therapy represents a therapeutic option for several monogenic diseases of the blood and the immune system as well as for storage disorders, and it may become a first-line treatment option for selected disease conditions. Another established cell and gene therapy application is adoptive immunotherapy, which exploits ex vivo expanded T cells, with or without genetic engineering to redirect their antigen specificity or to increase their safety profile, in order to harness the power of immune effector and regulatory cells for use against malignancies, infections and autoimmune diseases. A range of other types of somatic stem cells—in some cases involving genetic engineering—are showing promise for therapeutic applications, including epidermal and limbal stem cells, neural stem/progenitor cells (NSPCs), cardiac stem cells and multipotent stromal cells (MSCs).

Applications of ex-vivo therapy include reconstituting dysfunctional cell lineages. For inherited diseases characterized by a defective or absent cell lineage, the lineage can be regenerated by functional progenitor cells, derived either from normal donors or from autologous cells that have been subjected to ex vivo gene transfer to correct the deficiency. An example is provided by SCIDs, in which a deficiency in any one of several genes blocks the development of mature lymphoid cells. Transplantation of non-manipulated normal donor HSCs, which can allow generation of donor-derived functional hematopoietic cells of various lineages in the host, represents a therapeutic option for SCIDs, as well as many other diseases that affect the blood and immune system. Autologous HSC gene therapy, which can include replacing a functional copy of a defective gene in transplanted hematopoietic stem/progenitor cells (HSPCs) and, similarly to HCT, can provide a steady supply of functional progeny, may have several advantages, including reduced risk of graft versus host disease (GvHD), reduced risk of graft rejection, and reduced need for post-transplant immunosuppression.

Applications of ex-vivo therapy include augmenting therapeutic gene dosage. In some applications, HSC gene therapy may augment the therapeutic efficacy of allogenic HCT. Therapeutic gene dosage can be engineered to supra-normal levels in transplanted cells.

Applications of ex-vivo therapy include introducing novel function and targeting gene therapy. Ex vivo gene therapy can confer a novel function to HSCs or their progeny, such as establishing drug resistance to allow administration of a high-dose antitumor chemotherapy regime or establishing resistance to a pre-established infection with a virus, such as HIV, or other pathogen by expressing RNA-based agents (for example, ribozymes, RNA decoys, antisense RNA, RNA aptamers and small interfering RNA) and protein-based agents (for example, dominant-negative mutant viral proteins, fusion inhibitors and engineered nucleases that target the pathogen's genome).

Applications of ex-vivo therapy include enhancing immune responses. In neoplastic diseases, allogenic adaptive immune cell types, such as T cells, can recognize and kill cancer cells. Unfortunately, recognition of healthy tissues by alloreactive lymphocytes can also result in detrimental GvHD. Transfer of a suicide gene in donor lymphocytes allows their anti-tumor potential to be exploited, while taming their toxicity. In the autologous setting, lymphocytes with specificity directed against transformed or infected cells may be isolated from the patient's tissues and selectively expanded ex vivo. Alternatively, they may be generated by transfer of a gene for a synthetic or chimeric antigen receptor that triggers the cell's response when it encounters transformed or infected cells. These approaches may potentiate an underlying host response to a tumor or infection, or induce it de novo.

Therapeutic cell formulations and CD33-targeting agent compositions can be formulated for administration to subjects. In particular embodiments, cell-based formulations are administered to subjects as soon as reasonably possible following their initial formulation. In particular embodiments, formulations and/or compositions can be frozen (e.g., cryopreserved or lyophilized) prior to administration to a subject.

For example, as is understood by one of ordinary skill in the art, the freezing of cells can be destructive (see Mazur, P., 1977, Cryobiology 14:251-272) but there are numerous procedures available to prevent such damage. For example, damage can be avoided and/or reduced by (a) use of a cryoprotective agent, (b) control of the freezing rate, and/or (c) storage at a temperature sufficiently low to minimize degradative reactions. Exemplary cryoprotective agents include dimethyl sulfoxide (DMSO) (Lovelock and Bishop, 1959, Nature 183:1394-1395; Ashwood-Smith, 1961, Nature 190:1204-1205), glycerol, polyvinylpyrrolidine (Rinfret, 1960, Ann. N.Y. Acad. Sci. 85:576), polyethylene glycol (Sloviter and Ravdin, 1962, Nature 196:548), albumin, dextran, sucrose, ethylene glycol, i-erythritol, D-ribitol, D-mannitol (Rowe et al., 1962, Fed. Proc. 21:157), D-sorbitol, i-inositol, D-lactose, choline chloride (Bender et al., 1960, J. Appl. Physiol. 15:520), amino acids (Phan The Tran and Bender, 1960, Exp. Cell Res. 20:651), methanol, acetamide, glycerol monoacetate (Lovelock, 1954, Biochem. J. 56:265), and inorganic salts (Phan The Tran & Bender, 1960, Proc. Soc. Exp. Biol. Med. 104:388; Phan The Tran & Bender, 1961, in Radiobiology, Proceedings of the Third Australian Conference on Radiobiology, Ilbery ed., Butterworth, London, p. 59). In particular embodiments, DMSO can be used. Addition of plasma (e.g., to a concentration of 20-25%) can augment the protective effects of DMSO. After addition of DMSO, cells can be kept at 0° C. until freezing, because DMSO concentrations of 1% can be toxic at temperatures above 4° C.

In the cryopreservation of cells, slow controlled cooling rates can be critical and different cryoprotective agents (Rapatz et al., 1968, Cryobiology 5(1): 18-25) and different cell types have different optimal cooling rates (see e.g., Rowe & Rinfret, 1962, Blood 20:636; Rowe, 1966, Cryobiology 3(1):12-18; Lewis et al., 1967, Transfusion 7(1):17-32; and Mazur, 1970, Science 168:939-949 for effects of cooling velocity on survival of stem cells and on their transplantation potential). The heat of fusion phase where water turns to ice should be minimal. The cooling procedure can be carried out by use of, e.g., a programmable freezing device or a methanol bath procedure. Programmable freezing apparatuses allow determination of optimal cooling rates and facilitate standard reproducible cooling.

In particular embodiments, DMSO-treated cells can be pre-cooled on ice and transferred to a tray containing chilled methanol which is placed, in turn, in a mechanical refrigerator (e.g., Harris® (Thermo Fisher Scientific Inc., Waltham, Mass.) or Revco® (Thermo Fisher Scientific Inc., Waltham, Mass.)) at −80° C. Thermocouple measurements of the methanol bath and the samples indicate a cooling rate of 1° to 3° C./minute can be preferred. After at least two hours, the specimens can have reached a temperature of −80° C. and can be placed directly into liquid nitrogen (−196° C.).

After thorough freezing, the cells can be rapidly transferred to a long-term cryogenic storage vessel. In a preferred embodiment, samples can be cryogenically stored in liquid nitrogen (−196° C.) or vapor (−1° C.). Such storage is facilitated by the availability of highly efficient liquid nitrogen refrigerators.

Further considerations and procedures for the manipulation, cryopreservation, and long-term storage of cells, can be found in the following exemplary references: U.S. Pat. Nos. 4,199,022; 3,753,357; and 4,559,298; Gorin, 1986, Clinics In Haematology 15(1):19-48; Bone-Marrow Conservation, Culture and Transplantation, Proceedings of a Panel, Moscow, Jul. 22-26, 1968, International Atomic Energy Agency, Vienna, pp. 107-186; Livesey and Linner, 1987, Nature 327:255; Linner et al., 1986, J. Histochem. Cytochem. 34(9):1123-1135; Simione, 1992, J. Parenter. Sci. Technol. 46(6):226-32).

Following cryopreservation, frozen cells can be thawed for use in accordance with methods known to those of ordinary skill in the art. Frozen cells are preferably thawed quickly and chilled immediately upon thawing. In particular embodiments, the vial containing the frozen cells can be immersed up to its neck in a warm water bath; gentle rotation will ensure mixing of the cell suspension as it thaws and increase heat transfer from the warm water to the internal ice mass. As soon as the ice has completely melted, the vial can be immediately placed on ice.

In particular embodiments, methods can be used to prevent cellular clumping during thawing. Exemplary methods include: the addition before and/or after freezing of DNase (Spitzer et al., 1980, Cancer 45:3075-3085), low molecular weight dextran and citrate, hydroxyethyl starch (Stiff et al., 1983, Cryobiology 20:17-24), etc.

As is understood by one of ordinary skill in the art, if a cryoprotective agent that is toxic to humans is used, it should be removed prior to therapeutic use. In various embodiments, DMSO is regarded as a solvent that is suitable and/or safe for human use, and/or has no serious toxicity.

Exemplary carriers and modes of administration of cells are described at pages 14-15 of U.S. Patent Publication No. 2010/0183564. Additional pharmaceutical carriers are described in Remington: The Science and Practice of Pharmacy, 21st Edition, David B. Troy, ed., Lippicott Williams & Wilkins (2005).

In particular embodiments, cells can be harvested from a culture medium, and washed and concentrated into a carrier in a therapeutically-effective amount. Exemplary carriers include saline, buffered saline, physiological saline, water, Hanks' solution, Ringers solution, Nonnosol-R (Abbott Labs, Chicago, Ill.), Plasma-Lyte A® (Baxter Laboratories, Inc., Morton Grove, Ill.), glycerol, ethanol, and combinations thereof.

In particular embodiments, carriers can be supplemented with human serum albumin (HSA) or other human serum components or fetal bovine serum. In particular embodiments, a carrier for infusion includes buffered saline with 5% HAS or dextrose. Additional isotonic agents include polyhydric sugar alcohols including trihydric or higher sugar alcohols, such as glycerin, erythritol, arabitol, xylitol, sorbitol, or mannitol.

Carriers can include buffering agents, such as citrate buffers, succinate buffers, tartrate buffers, fumarate buffers, gluconate buffers, oxalate buffers, lactate buffers, acetate buffers, phosphate buffers, histidine buffers, and/or trimethylamine salts.

Stabilizers refer to a broad category of excipients which can range in function from a bulking agent to an additive which helps to prevent cell adherence to container walls. Typical stabilizers can include polyhydric sugar alcohols; amino acids, such as arginine, lysine, glycine, glutamine, asparagine, histidine, alanine, ornithine, L-leucine, 2-phenylalanine, glutamic acid, and threonine; organic sugars or sugar alcohols, such as lactose, trehalose, stachyose, mannitol, sorbitol, xylitol, ribitol, myoinisitol, galactitol, glycerol, and cyclitols, such as inositol; PEG; amino acid polymers; sulfur-containing reducing agents, such as urea, glutathione, thioctic acid, sodium thioglycolate, thioglycerol, alpha-monothioglycerol, and sodium thiosulfate; low molecular weight polypeptides (i.e., <10 residues); proteins such as HSA, bovine serum albumin, gelatin or immunoglobulins; hydrophilic polymers such as polyvinylpyrrolidone; monosaccharides such as xylose, mannose, fructose and glucose; disaccharides such as lactose, maltose and sucrose; trisaccharides such as raffinose, and polysaccharides such as dextran.

Where necessary or beneficial, compositions or formulations can include a local anesthetic such as lidocaine to ease pain at a site of injection.

Exemplary preservatives include phenol, benzyl alcohol, meta-cresol, methyl paraben, propyl paraben, octadecyldimethylbenzyl ammonium chloride, benzalkonium halides, hexamethonium chloride, alkyl parabens such as methyl or propyl paraben, catechol, resorcinol, cyclohexanol, and 3-pentanol.

Therapeutically effective amounts of cells within cell-based formulations can be greater than 10² cells, greater than 10³ cells, greater than 10⁴ cells, greater than 10⁵ cells, greater than 10⁶ cells, greater than 10⁷ cells, greater than 10⁸ cells, greater than 10⁹ cells, greater than 10¹⁰ cells, or greater than 10¹¹ cells. In cell-based formulations disclosed herein, cells are generally in a volume of a liter or less, 500 ml or less, 250 ml or less, or 100 ml or less. Hence the density of administered cells is typically greater than 10⁴ cells/ml, 10⁷ cells/ml or 10⁸ cells/ml.

Therapeutically effective amounts of protein-based compounds within CD33 targeting compositions can include 0.1 to 5 μg or μg/mL or L, or from 0.5 to 1 μg or μg/mL or L. In other examples, a dose can include 1 μg or μg/mL or L, 15 μg or μg/mL or L, 30 μg or μg/mL or L, 50 μg or μg/mL or L, 55 μg or μg/mL or L, 70 μg or μg/mL or L, 90 μg or μg/mL or L, 150 μg or μg/mL or L, 350 μg or μg/mL or L, 500 μg or μg/mL or L, 750 μg or μg/mL or L, 1000 μg or μg/mL or L, 0.1 to 5 mg/mL or L or from 0.5 to 1 mg/mL or L. In other examples, a dose can include 1 mg/mL or L, 10 mg/mL or L, 30 mg/mL or L, 50 mg/mL or L, 70 mg/mL or L, 100 mg/mL or L, 300 mg/mL or L, 500 mg/mL or L, 700 mg/mL or L, 1000 mg/mL or L or more.

Cell formulations and CD33 targeting compositions can be prepared for administration by, for example, injection, infusion, perfusion, or lavage. CD33-targeting agent compositions can also be prepared as oral, inhalable, or implantable compositions.

Vectors and formulations disclosed herein can be used for treating subjects (e.g., humans, veterinary animals (e.g., dogs, cats, reptiles, birds, etc.), livestock (e.g., horses, cattle, goats, pigs, chickens, etc.), and research animals (e.g., non-human primates, monkeys, rats, mice, fish, etc.). In particular embodiments, subjects are human. Treating subjects includes delivering therapeutically effective amounts of one or more vectors, genomes, or systems of the present disclosure. Therapeutically effective amounts include those that provide effective amounts, prophylactic treatments, and/or therapeutic treatments.

Various formulations are known in the art for each reagent and/or technique of delivering a heterologous nucleic acid to a target cell.

### Anti-CD33 Agents

The present disclosure includes, among other things, administering an anti-CD33 agent to a subject or system that includes one or more CD33-expressing and/or CD33-inactivated (a.k.a., CD33-disrupted) cells.

An anti-CD33 agent (also referred to herein by the synonymous term CD33-targeting agent) can refer to a molecule, cell, drug, or combination thereof that targets CD33-expressing cells for cell death or to inhibit cell growth. CD33-targeting agents include molecules that result in the elimination of CD33-expressing cells. Examples of CD33-targeting agents include anti-CD33 antibodies; anti-CD33 immunotoxins; anti-CD33 antibody-drug conjugates; anti-CD33 antibody-radioisotope conjugates; anti-CD33 multispecific antibodies (e.g. bispecific and trispecific antibodies, e.g., bispecific and trispecific antibodies that bind CD33 and an immune-activating epitope on an immune cell (e.g., CD3 as in BiTE®); and/or immune cells expressing CARs or engineered TCRs that specifically bind CD33. Anti-CD33 antibodies are described above in relation to binding domains. Each of these types of CD33-targeting agents include a binding domain that binds CD33, and most (e.g., except certain antibody forms) also include a linker. Accordingly, CD33 binding domains are described first and a general description of linkers is provided next. Following this description of CD33 binding domains and linkers, more particular information regarding the different CD33-targeting agents is provided.

CD33 Binding Domains. Binding domains include any substance that binds to CD33 to form a complex. The choice of binding domain can depend upon the type and number of CD33 markers that define the surface of a target cell or the type of selected CD33-targeting agent. Examples of binding domains include cellular marker ligands, receptor ligands, antibodies, antibody binding domains, peptides, peptide aptamers, receptors (e.g., T cell receptors), or combinations and engineered fragments or formats thereof.

Antibodies are one example of binding domains and include whole antibodies or binding fragments of an antibody, e.g., Fv, Fab, Fab′, F(ab′)2, and single chain (sc) forms and fragments thereof that bind specifically CD33. Antibodies or antigen binding fragments can include all or a portion of polyclonal antibodies, monoclonal antibodies, human antibodies, humanized antibodies, synthetic antibodies, non-human antibodies, recombinant antibodies, chimeric antibodies, bispecific antibodies, mini bodies, and linear antibodies.

Antibodies can be produced from two genes, a heavy chain gene and a light chain gene. Generally, an antibody can include two identical copies of a heavy chain, and two identical copies of a light chain. Within a variable heavy chain and variable light chain, segments referred to as complementary determining regions (CDRs) dictate epitope binding. Each heavy chain has three CDRs (i.e., CDRH1, CDRH2, and CDRH3) and each light chain has three CDRs (i.e., CDRL1, CDRL2, and CDRL3). CDR regions are flanked by framework residues (FR).

Anti-CD33 bispecific antibodies bind at least two epitopes wherein at least one of the epitopes is located on CD33. Anti-CD33 trispecific antibodies bind at least 3 epitopes, wherein at least one of the epitopes is located on CD33.

Some examples of bispecific antibodies have two heavy chains (each having three heavy chain CDRs, followed by (N-terminal to C-terminal) a CH1 domain, a hinge, a CH2 domain, and a CH3 domain), and two immunoglobulin light chains that confer antigen-binding specificity through association with each heavy chain. However, additional architectures can be used, including bispecific antibodies in which the light chain(s) associate with each heavy chain but do not (or minimally) contribute to antigen-binding specificity, or that can bind one or more of the epitopes bound by the heavy chain antigen-binding regions, or that can associate with each heavy chain and enable binding of one or both of the heavy chains to one or both epitopes. Other forms of bispecific antibodies include the single chain “Janusins” described in Traunecker et al. (Embo Journal, 10, 3655-3659, 1991).

Bispecific antibodies can be prepared as full-length antibodies or antibody fragments (for example, F(ab′)₂ bispecific antibodies).

Methods for making bispecific antibodies are also described in Millstein et al. Nature 305:37-39, 1983; WO 1993/008829; and Traunecker et al., EMBO J. 10:3655-3659, 1991. In particular embodiments, bispecific antibodies can be prepared using chemical linkage. For example, Brennan et al. (Science 229: 81, 1985) describes a procedure wherein intact antibodies are proteolytically cleaved to generate F(ab′)₂ fragments. These fragments are reduced in the presence of the dithiol complexing agent, sodium arsenite, to stabilize vicinal dithiols and prevent intermolecular disulfide formation. The Fab′ fragments generated then are converted to thionitrobenzoate (TNB) derivatives. One of the Fab′-TNB derivatives then is reconverted to the Fab′-thiol by reduction with mercaptoethylamine and is mixed with an equimolar amount of the other Fab′-TNB derivative to form the bispecific antibody.

In particular embodiments, the disclosure provides proteins that bind with a cognate binding molecule with an association rate constant or kₒₙ rate of not more than 10⁷ M⁻¹ s⁻¹, less than 5×10⁶ M⁻¹ s⁻¹, less than 2.5×10⁶ M⁻¹ s⁻¹, less than 2×10⁶ M⁻¹ s⁻¹, less than 1.5×10⁶ M⁻¹ s⁻¹, less than 10⁶ M⁻¹ s⁻¹, less than 5×10⁵ M⁻¹ s⁻¹, less than 2.5×10⁵ M⁻¹ s⁻¹, less than 2×10⁵ M⁻¹ s⁻¹, less than 1.5×10⁵ M⁻¹ s⁻¹, less than 10⁵ M⁻¹ s⁻¹, less than 5×10⁴ M⁻¹ s⁻¹, less than 2.5×10⁴ M⁻¹ s⁻¹, less than 2×10⁴ M⁻¹ s⁻¹, less than 1.5×10⁴ M's′, less than 10⁴ M's′, less than 10³ M⁻¹ s⁻¹, less than 10² M⁻¹ s⁻¹, or in a range of 10² M⁻¹ s⁻¹ to 10⁷ M⁻¹ s⁻¹, in a range of 10³ M⁻¹ s⁻¹ to 10⁶ M⁻¹ s⁻¹, in a range of 10⁴ M⁻¹ s⁻¹ to 10⁵ M⁻¹ s⁻¹, or in a range of 10³ M⁻¹ s⁻¹ to 10⁷ M⁻¹ s⁻¹.

In particular embodiments, the disclosure provides proteins that bind with a cognate binding molecule a k_{off} rate of not less than 0.5 s⁻¹, not less than 0.25 s⁻¹, not less than 0.2 s⁻¹, not less than 0.1 s⁻¹, not less than 5×10⁻² s⁻¹, not less than 2.5×10⁻² s⁻¹, not less than 2×10⁻² s⁻¹, not less than 1.5×10⁻² s⁻¹, not less than 10⁻² s⁻¹, not less than 5×10⁻³ s⁻¹, not less than 2.5×10⁻³ s⁻¹, not less than 2×10⁻³ s⁻¹, not less than 1.5×10⁻³ s⁻¹, not less than 10⁻³ s⁻¹, not less than 5×10⁻⁴ s⁻¹, not less than 2.5×10⁻⁴ s⁻¹, not less than 2×10⁻⁴ s⁻¹, not less than 1.5×10⁻⁴ s⁻¹, not less than 10⁻⁴ s⁻¹, not less than 5×10⁻⁵ s⁻¹, not less than 2.5×10⁻⁵ s⁻¹, not less than 2×10⁻⁵ s⁻¹, not less than 1.5×10⁻⁵ s⁻¹, not less than 10⁻⁵ s⁻¹, not less than 5×10⁻⁶ s⁻¹, not less than 2.5×10⁻⁶ s⁻¹, not less than 2×10⁻⁶ s⁻¹, not less than 1.5×10⁻⁶ s⁻¹, not less than 10⁻⁶ s⁻¹, or in a range of 0.5 to 10⁻⁶ s⁻¹, in a range of 10⁻² s⁻¹ to 10⁻⁵ s⁻¹, or in a range of 10⁻³ s⁻¹ to 10⁻⁴ s⁻¹.

In particular embodiments, the disclosure provides proteins that bind with a cognate binding molecule with an affinity constant or Kₐ (kₒₙ/k_{off}) of, either before and/or after modification, less than 10⁶ M⁻¹, less than 5×10⁵ M⁻¹, less than 2.5×10⁵ M⁻¹, less than 2×10⁵ M⁻¹, less than 1.5×10⁵ M⁻¹, less than 10⁵ M⁻¹, less than 5×10⁴ M⁻¹, less than 2.5×10⁴ M⁻¹, less than 2×10⁴ M⁻¹, less than 1.5×10⁴ M⁻¹, less than 10⁴ M⁻¹, less than 5×10³ M⁻¹, less than 2.5×10³ M⁻¹, less than 2×10³ M⁻¹, less than 1.5×10³ M⁻¹, less than 10³ M⁻¹, less than 500 M⁻¹, less than 250 M⁻¹, less than 200 M⁻¹, less than 150 M⁻¹, less than 100 M⁻¹, less than 50 M⁻¹, less than 25 M⁻¹, less than 20 M⁻¹, less than 15 M⁻¹, or less than 10 M⁻¹, or in a range of 10 M⁻¹ to 10⁶ M⁻¹, in a range of 10² M⁻¹ to 10⁵ M⁻¹, or in a range of 10³ M⁻¹ to 1×10⁴ M⁻¹.

In particular embodiments, the disclosure provides proteins that bind with a cognate binding molecule with a dissociation constant or K_{d} (k_{off}/kₒₙ) of, either before and/or after modification, not less than 0.05 M, not less than 0.025 M, not less than 0.02 M, not less than 0.01 M, not less than 5×10⁻³ M, not less than 2.5×10⁻³ M, not less than 2×10⁻³ M, not less than 1.5×10⁻³ M, not less than 10⁻³ M, not less than 5×10⁻⁴ M, not less than 2.5×10⁻⁴ M, not less than 2×10⁻⁴ M, not less than 1.5×10⁻⁴ M, not less than 10⁻⁴ M, not less than 5×10⁻⁵ M, not less than 2.5×10⁻⁵ M, not less than 2×10⁻⁵ M, not less than 1.5×10⁻⁵ M, not less than 10⁻⁵ M, not less than 5×10⁻⁶ M, not less than 2.5×10⁻⁶ M, not less than 2×10⁻⁶ M, not less than 1.5×10⁻⁶ M, not less than 10⁻⁶ M, or not less than 10⁻⁷ M, or in a range of 0.05 M to 10⁻⁷ M, in a range of 5×10⁻³ M to 10⁻⁶ M, or in a range of 10⁻⁴ M to 10⁻′M.

When antibody residues are provided, the assignment of amino acids to each domain is in accordance with Kabat Sequences of Proteins of Immunological Interest (National Institutes of Health, Bethesda, Md. (1987 and 1991)) unless otherwise specified.

Unless otherwise indicated, aspects of the present disclosure can employ conventional techniques of immunology, molecular biology, microbiology, cell biology and recombinant DNA. These methods are described in the following publications. See, e.g., Sambrook et al. Molecular Cloning: A Laboratory Manual, 2nd Edition (1989); F. M. Ausubel et al. eds., Current Protocols in Molecular Biology, (1987); the series Methods IN Enzymology (Academic Press, Inc.); M. MacPherson et al., PCR: A Practical Approach, IRL Press at Oxford University Press (1991); MacPherson et al., eds. PCR 2: Practical Approach, (1995); Harlow and Lane, eds. Antibodies, A Laboratory Manual, (1988); and R. I. Freshney, ed. Animal Cell Culture (1987).

In particular embodiments, the CD33 binding domain can be derived from or include hP67.6 which is an anti-CD33 antibody used in the ADC, GO. In particular embodiments, the light chain of hP67.6 includes:

(SEQ ID NO: 32)
MSVPTQVLGLLLLWLTDARCDIQLTQSPSTLSASVGDRVTITCRASESL
DNYGIRFLTWFQQKPGKAPKLLMYAASNQGSGVPSRFSGSGSGTEFTLT
ISSLQPDDFATYYCQQTKEVPWSFGQGTKVEVKRT

and the heavy chain of hP67.6 includes:

(SEQ ID NO: 33)
MEWSWVFLFFLSVTTGVHSEVQLVQSGAEVKKPGSSVKVSCKASGYTIT
DSNIHWVRQAPGQSLEWIGYIYPYNGGTDYNQKFKNRATLTVDNPTNTA
YMELSSLRSEDTDFYYCVNGNPWLAYWGQGTLVTVSSASTKGP.

In particular embodiments, the hP67.6 binding domain includes a variable light chain including a CDRL1 sequence including QSPSTLSASV (SEQ ID NO: 34), a CDRL2 sequence including DNYGIRFLTWFQQKPG (SEQ ID NO: 35), and a CDRL3 sequence including FTLTISSL (SEQ ID NO: 36). In particular embodiments, the hP67.6 binding domain includes a variable heavy chain including a CDRH1 sequence including VQSGAEVKKPG (SEQ ID NO: 37), a CDRH2 sequence including DSNIHWV (SEQ ID NO: 38), and a CDRH3 sequence including LTVDNPTNT (SEQ ID NO: 39).

In particular embodiments, the CD33 binding domain can be derived from or include h2H12EC which is the anti-CD33 antibody used in the ADC, SGN-CD33A. In particular embodiments, the h2H12EC binding domain includes a variable light chain including a CDRL1 sequence including NYDIN (SEQ ID NO: 40), a CDRL2 sequence including WIYPGDGSTKYNEKFKA (SEQ ID NO: 41), and a CDRL3 sequence including GYEDAMDY (SEQ ID NO: 42). In particular embodiments, the h2H12EC binding domain includes a variable heavy chain including a CDRH1 sequence including KASQDINSYLS (SEQ ID NO: 43), a CDRH2 sequence including RANRLVD (SEQ ID NO: 44), and a CDRH3 sequence including LQYDEFPLT (SEQ ID NO: 45).

Additional examples of anti-CD33 antibody heavy and light chains, as well as specific CDRs, include those described in U.S. Pat. No. 7,557,189. For instance, in particular embodiments, a light chain of a representative anti-CD33 antibody includes:

(SEQ ID NO: 46)
NIMLTQSPSSLAVSAGEKVTMSCKSSQSVFFSSSQKNYLAWYQQIPGQS
PKLLIYWASTRESGVPDRFTGSGSGTDFTLTISSVQSEDLAIYYCHQYL
SSRTFGGGTKLEIKR

and a heavy chain of this representative anti-CD33 antibody includes:

(SEQ ID NO: 47)
QVQLQQPGAEVVKPGASVKMSCKASGYTFTSYYIHWIKQTPGQGLEWVG
VIYPGNDDISYNQKFKGKATLTADKSSTTAYMQLSSLTSEDSAVYYCAR
EVRLRYFDVWGAGTTVTVSS .

In particular embodiments, the CD33 binding domain includes a variable light chain including a CDRL1 sequence including SYYIH (SEQ ID NO: 105), a CDRL2 sequence including VIYPGNDDISYNQKFXG (SEQ ID NO: 48) wherein X is K or Q, and a CDRL3 sequence including EVRLRYFDV (SEQ ID NO: 49). In particular embodiments, the CD33 binding domain includes a variable heavy chain including a CDRH1 sequence including KSSQSVFFSSSQKNYLA (SEQ ID NO: 50), a CDRH2 sequence including WASTRES (SEQ ID NO: 51), and a CDRH3 sequence including HQYLSSRT (SEQ ID NO: 52).

In some instances, it is beneficial for the binding domain to be derived from the same species it will ultimately be used in. For example, for use in humans, it may be beneficial for the antigen binding domain to include a human antibody, humanized antibody, or a fragment or engineered form thereof. Antibodies from human origin or humanized antibodies have lowered or no immunogenicity in humans and have a lower number of non-immunogenic epitopes compared to non-human antibodies. Antibodies and their engineered fragments will generally be selected to have a reduced level or no antigenicity in human subjects.

In particular embodiments, the binding domain includes a humanized antibody or an engineered fragment thereof. In particular embodiments, a non-human antibody is humanized, where one or more amino acid residues of the antibody are modified to increase similarity to an antibody naturally produced in a human or fragment thereof. These nonhuman amino acid residues are often referred to as “import” residues, which are typically taken from an “import” variable domain. As provided herein, humanized antibodies or antibody fragments include one or more CDRs from nonhuman immunoglobulin molecules and framework regions wherein the amino acid residues including the framework are derived completely or mostly from human germline. In one aspect, the antigen binding domain is humanized. A humanized antibody can be produced using a variety of techniques known in the art, including CDR-grafting (see, e.g., European Patent No. EP 239,400; WO 91/09967; and U.S. Pat. Nos. 5,225,539, 5,530,101, and 5,585,089), veneering or resurfacing (see, e.g., EP 592,106 and EP 519,596; Padlan, 1991, Molecular Immunology, 28(4/5):489-498; Studnicka et al., 1994, Protein Engineering, 7(6):805-814; and Roguska et al., 1994, PNAS, 91:969-973), chain shuffling (see, e.g., U.S. Pat. No. 5,565,332), and techniques disclosed in, e.g., US 2005/0042664, US 2005/0048617, U.S. Pat. Nos. 6,407,213, 5,766,886, WO 9317105, Tan et al., J. Immunol., 169:1119-25 (2002), Caldas et al., Protein Eng., 13(5):353-60 (2000), Morea et al., Methods, 20(3):267-79 (2000), Baca et al., J. Biol. Chem., 272(16): 10678-84 (1997), Roguska et al., Protein Eng., 9(10):895-904 (1996), Couto et al., Cancer Res., 55 (23 Supp):5973s-5977s (1995), Couto et al., Cancer Res., 55(8):1717-22 (1995), Sandhu J S, Gene, 150(2):409-10 (1994), and Pedersen et al., J. Mol. Biol., 235(3):959-73 (1994). Often, framework residues in the framework regions will be substituted with the corresponding residue from the CDR donor antibody to alter, for example improve, cellular marker binding. These framework substitutions are identified by methods well-known in the art, e.g., by modeling of the interactions of the CDR and framework residues to identify framework residues important for cellular marker binding and sequence comparison to identify unusual framework residues at particular positions. (See, e.g., U.S. Pat. No. 5,585,089; and Riechmann et al., Nature, 332:323, 1988)

Antibodies with binding domains that specifically bind CD33 can be prepared using methods of obtaining monoclonal antibodies, methods of phage display, methods to generate human or humanized antibodies, or methods using a transgenic animal or plant engineered to produce antibodies as is known to those of ordinary skill in the art (see, for example, U.S. Pat. Nos. 6,291,161 and 6,291,158). Phage display libraries of partially or fully synthetic antibodies are available and can be screened for an antibody or fragment thereof that can bind to CD33. For example, binding domains may be identified by screening a Fab phage library for Fab fragments that specifically bind CD33 (see Hoet et al., Nat. Biotechnol. 23:344, 2005). Phage display libraries of human antibodies are also available. Additionally, traditional strategies for hybridoma development using CD33 as an immunogen in convenient systems (e.g., mice, HuMAb Mouse® (GenPharm Intl Inc., Mountain View, Calif.), TC Mouse® (Kirin Pharma Co. Ltd., Tokyo, JP), KM-Mouse® (Medarex, Inc., Princeton, N.J.), llamas, chicken, rats, hamsters, rabbits, etc.) can be used to develop binding domains. Once identified, the amino acid sequence of the antibody and gene sequence encoding the antibody can be isolated and/or determined.

As indicated, antibodies can be used as whole antibodies or binding fragments thereof, e.g., Fv, Fab, Fab′, F(ab′)₂, and single chain (sc) forms and fragments thereof that specifically bind CD33.

In some instances, scFvs can be prepared according to methods known in the art (see, for example, Bird et al., (1988) Science 242:423-426 and Huston et al., (1988) Proc. Natl. Acad. Sci. USA 85:5879-5883). ScFv molecules can be produced by linking VH and VL regions of an antibody together using flexible polypeptide linkers. If a short polypeptide linker is employed (e.g., between 5-10 amino acids) intrachain folding is prevented. Interchain folding is also required to bring the two variable regions together to form a functional epitope binding site. For examples of linker orientations and sizes see, e.g., Hollinger et al. 1993 Proc Natl Acad. Sci. U.S.A. 90:6444-6448, US 2005/0100543, US 2005/0175606, US 2007/0014794, and WO2006/020258 and WO2007/024715. More particularly, linker sequences that are used to connect the VL and VH of an scFv are generally five to 35 amino acids in length. In particular embodiments, a VL-VH linker includes from five to 35, ten to 30 amino acids or from 15 to 25 amino acids. Variation in the linker length may retain or enhance activity, giving rise to superior efficacy in activity studies. scFV are commonly used as the binding domains of CAR discussed below.

Additional examples of antibody-based binding domain formats include scFv-based grababodies and soluble VH domain antibodies. These antibodies form binding regions using only heavy chain variable regions. See, for example, Jespers et al., Nat. Biotechnol. 22:1161, 2004; Cortez-Retamozo et al., Cancer Res. 64:2853, 2004; Baral et al., Nature Med. 12:580, 2006; and Barthelemy et al., J. Biol. Chem. 283:3639, 2008.

In particular embodiments, a VL region in a binding domain of the present disclosure is derived from or based on a VL of a known monoclonal antibody and contains one or more (e.g., 2, 3, 4, 5, 6, 7, 8, 9, 10) insertions, one or more (e.g., 2, 3, 4, 5, 6, 7, 8, 9, 10) deletions, one or more (e.g., 2, 3, 4, 5, 6, 7, 8, 9, 10) amino acid substitutions (e.g., conservative amino acid substitutions), or a combination of the above-noted changes, when compared with the VL of the known monoclonal antibody. An insertion, deletion or substitution may be anywhere in the VL region, including at the amino- or carboxy-terminus or both ends of this region, provided that each CDR includes zero changes or at most one, two, or three changes and provided a binding domain containing the modified VL region can still specifically bind its target with an affinity similar to the wild type binding domain.

In particular embodiments, a binding domain VH region of the present disclosure can be derived from or based on a VH of a known monoclonal antibody and can contain one or more (e.g., 2, 3, 4, 5, 6, 7, 8, 9, 10) insertions, one or more (e.g., 2, 3, 4, 5, 6, 7, 8, 9, 10) deletions, one or more (e.g., 2, 3, 4, 5, 6, 7, 8, 9, 10) amino acid substitutions (e.g., conservative amino acid substitutions or non-conservative amino acid substitutions), or a combination of the above-noted changes, when compared with the VH of a known monoclonal antibody. An insertion, deletion or substitution may be anywhere in the VH region, including at the amino- or carboxy-terminus or both ends of this region, provided that each CDR includes zero changes or at most one, two, or three changes and provided a binding domain containing the modified VH region can still specifically bind its target with an affinity similar to the wild type binding domain.

In particular embodiments, a binding domain includes or is a sequence that is at least 90%, at least 91%, at least 92%, at least 93%, at least 94%, at least 95%, at least 96%, at least 97%, at least 98%, at least 99%, at least 99.5%, or 100% identical to an amino acid sequence of a light chain variable region (VL) or to a heavy chain variable region (VH), or both, wherein each CDR includes zero changes or at most one, two, or three changes, from a monoclonal antibody or fragment or derivative thereof that specifically binds to a cellular marker of interest.

An alternative source of binding domains includes sequences that encode random peptide libraries or sequences that encode an engineered diversity of amino acids in loop regions of alternative non-antibody scaffolds, such as single chain (sc) T-cell receptor (scTCR) (see, e.g., Lake et al., Int. Immunol. 11:745, 1999; Maynard et al., J. Immunol. Methods 306:51, 2005; U.S. Pat. No. 8,361,794), fibrinogen domains (see, e.g., Weisel et al., Science 230:1388, 1985), Kunitz domains (see, e.g., U.S. Pat. No. 6,423,498), designed ankyrin repeat proteins (DARPins; Binz et al., J. Mol. Biol. 332:489, 2003 and Binz et al., Nat. Biotechnol. 22:575, 2004), fibronectin binding domains (adnectins or monobodies; Richards et al., J. Mol. Biol. 326:1475, 2003; Parker et al., Protein Eng. Des. Selec. 18:435, 2005 and Hackel et al. (2008) J. Mol. Biol. 381:1238-1252), cysteine-knot miniproteins (Vita et al., 1995, Proc. Nat'l. Acad. Sci. (USA) 92:6404-6408; Martin et al., 2002, Nat. Biotechnol. 21:71, 2002 and Huang et al. (2005) Structure 13:755, 2005), tetratricopeptide repeat domains (Main et al., Structure 11:497, 2003 and Cortajarena et al., ACS Chem. Biol. 3:161, 2008), leucine-rich repeat domains (Stumpp et al., J. Mol. Biol. 332:471, 2003), lipocalin domains (see, e.g., WO 2006/095164, Beste et al., Proc. Nat'l. Acad. Sci. (USA) 96:1898, 1999 and Schonfeld et al., Proc. Nat'l. Acad. Sci. (USA) 106:8198, 2009), V-like domains (see, e.g., US 2007/0065431), C-type lectin domains (Zelensky and Gready, FEBS J. 272:6179, 2005; Beavil et al., Proc. Nat'l. Acad. Sci. (USA) 89:753, 1992 and Sato et al., Proc. Nat'l. Acad. Sci. (USA) 100:7779, 2003), mAb2 or Fc-region with antigen binding domain (Fcab™ (F-Star Biotechnology, Cambridge UK; see, e.g., WO 2007/098934 and WO 2006/072620), armadillo repeat proteins (see, e.g., Madhurantakam et al., Protein Sci. 21: 1015, 2012; WO 2009/040338), affilin (Ebersbach et al., J. Mol. Biol. 372: 172, 2007), affibody, avimers, knottins, fynomers, atrimers, cytotoxic T-lymphocyte associated protein-4 (Weidle et al., Cancer Gen. Proteo. 10:155, 2013), or the like (Nord et al., Protein Eng. 8:601, 1995; Nord et al., Nat. Biotechnol. 15:772, 1997; Nord et al., Euro. J. Biochem. 268:4269, 2001; Binz et al., Nat. Biotechnol. 23:1257, 2005; Boersma and Pluckthun, Curr. Opin. Biotechnol. 22:849, 2011).

Peptide aptamers include a peptide loop (which is specific for a cellular marker) attached at both ends to a protein scaffold. This double structural constraint increases the binding affinity of peptide aptamers to levels comparable to antibodies. The variable loop length is typically 8 to 20 amino acids and the scaffold can be any protein that is stable, soluble, small, and non-toxic. Peptide aptamer selection can be made using different systems, such as the yeast two-hybrid system (e.g., Gal4 yeast-two-hybrid system), or the LexA interaction trap system.

In particular embodiments, a binding domain is a scTCR including Vα/β and Cα/β chains (e.g., Vα-Cα, Vβ-Cβ, Vα-Vβ) or including a Vα-Cα, Vβ-Cβ, Vα-Vβ pair specific for a CD33 peptide-MHC complex.

In particular embodiments, engineered binding domains include Vα, Vβ, Cα, or Cβ regions derived from or based on a Vα, Vβ, Cα, or Cβ and includes one or more (e.g., 2, 3, 4, 5, 6, 7, 8, 9, 10) insertions, one or more (e.g., 2, 3, 4, 5, 6, 7, 8, 9, 10) deletions, one or more (e.g., 2, 3, 4, 5, 6, 7, 8, 9, 10) amino acid substitutions (e.g., conservative amino acid substitutions or non-conservative amino acid substitutions), or a combination of the above-noted changes, when compared with the referenced Vα, Vβ, Cα, or Cβ. An insertion, deletion or substitution may be anywhere in a V_{L}, V_{H}, Vα, Vβ, Cα, or Cβ region, including at the amino- or carboxy-terminus or both ends of these regions, provided that each CDR includes zero changes or at most one, two, or three changes and provides a target binding domain containing a modified Vα, Vβ, Cα, or Cβ region can still specifically bind its target with an affinity and action similar to wild type.

In particular embodiments, engineered binding domains include a sequence that is at least 90%, at least 91%, at least 92%, at least 93%, at least 94%, at least 95%, at least 96%, at least 97%, at least 98%, at least 99%, at least 99.5%, or 100% identical to an amino acid sequence of a known or identified binding domain, wherein each CDR includes zero changes or at most one, two, or three changes, from a known or identified binding domain or fragment or derivative thereof that specifically binds to the targeted cellular marker.

The precise amino acid sequence boundaries of a given CDR or FR can be readily determined using any of a number of well-known schemes, including those described by: Kabat et al. (1991) “Sequences of Proteins of Immunological Interest,” 5th Ed. Public Health Service, National Institutes of Health, Bethesda, Md. (Kabat numbering scheme); A1-Lazikani et al. (1997) J Mol Biol 273: 927-948 (Chothia numbering scheme); Maccallum et al. (1996) J Mol Biol 262: 732-745 (Contact numbering scheme); Martin et al. (1989) Proc. Natl. Acad. Sci., 86: 9268-9272 (AbM numbering scheme); Lefranc et al. (2003) Dev Comp Immunol 27(1): 55-77 (IMGT numbering scheme); and Honegger and Pluckthun (2001) J Mol Biol 309(3): 657-670 (“Aho” numbering scheme). The boundaries of a given CDR or FR may vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme. In particular embodiments, the antibody CDR sequences disclosed herein are according to Kabat numbering.

As indicated, many CD33-targeting agents include linkers. Linkers can be used to achieve different outcomes depending on the particular CD33-targeting agent under consideration. A linker can include any chemical moiety that is capable of linking portions of a CD33-targeting agent. Linkers can be flexible, rigid, or semi-rigid, depending on the desired function of the linker. Examples of anti-CD33 agents that can include a linker include, without limitation, bispecific antibodies.

For example, in particular embodiments, linkers provide flexibility and room for conformational movement between different components of CD33-targeting agents. Commonly used flexible linkers include linker sequence with the amino acids glycine and serine (Gly-Ser linkers). In particular embodiments, the linker sequence includes sets of glycine and serine repeats such as from one to ten repeats of (GlyₓSer_{y})ₙ, wherein x and y are independently an integer from 0 to 10 provided that x and y are not both 0 and wherein n is an integer of 1, 2, 3, 4, 5, 6, 7, 8, 9 or 10). Particular examples include

(SEQ ID NO: 53)
(Gly4Ser)n,
(SEQ ID NO: 54)
(Gly3Ser)n(Gly4Ser)n,
(SEQ ID NO: 55)
(Gly3Ser)n(Gly2Ser)n,
or
(SEQ ID NO: 56)
(Gly3Ser)n(Gly4Ser)₁.
In particular embodiments, the linker is
(SEQ ID NO: 57)
(Gly4Ser)₄,
(SEQ ID NO: 58)
(Gly4Ser)₃,
(SEQ ID NO: 59)
(Gly4Ser)₂,
(SEQ ID NO: 60)
(Gly4Ser)₁,
(SEQ ID NO: 61)
(Gly3Ser)₂,
(SEQ ID NO: 62)
(Gly3Ser)₁,
(SEQ ID NO: 16)
(Gly2Ser)₂,
(Gly2Ser)₁,
(SEQ ID NO: 63)
GGSGGGSGGSG,
(SEQ ID NO: 64)
GGSGGGSGSG,
or
(SEQ ID NO: 65)
GGSGGGSG.

In some situations, flexible linkers may be incapable of maintaining a distance or positioning of CD33-targeting agent components needed for a particular use. In these instances, rigid or semi-rigid linkers may be useful. Examples of rigid or semi-rigid linkers include proline-rich linkers. In particular embodiments, a proline-rich linker is a peptide sequence having more proline residues than would be expected based on chance alone. In particular embodiments, a proline-rich linker is one having at least 30%, at least 35%, at least 36%, at least 39%, at least 40%, at least 48%, at least 50%, or at least 51% proline residues. Particular examples of proline-rich linkers include fragments of proline-rich salivary proteins (PRPs).

Spacer regions are a type of linker region that are used to create appropriate distances and/or flexibility from other linked components. In particular embodiments, the length of a spacer region can be customized for individual cellular markers on unwanted cells to optimize unwanted CD33-expressing cell recognition and destruction. The spacer can be of a length that provides for increased effectiveness of the CD33-targeting agent following CD33 binding, as compared to in the absence of the spacer. In particular embodiments, a spacer region length can be selected based upon the location of a cellular marker epitope, affinity of a binding domain for the epitope, and/or the ability of the CD33-targeting agent to mediate cell destruction following CD33 binding.

Spacer regions typically include those having 10 to 250 amino acids, 10 to 200 amino acids, 10 to 150 amino acids, 10 to 100 amino acids, 10 to 50 amino acids, or 10 to 25 amino acids. In particular embodiments, a spacer region is 12 amino acids, 20 amino acids, 21 amino acids, 26 amino acids, 27 amino acids, 45 amino acids, or 50 amino acids.

Exemplary spacer regions include all or a portion of an immunoglobulin hinge region. An immunoglobulin hinge region may be a wild-type immunoglobulin hinge region or an altered wild-type immunoglobulin hinge region. In certain embodiments, an immunoglobulin hinge region is a human immunoglobulin hinge region. As used herein, a “wild type immunoglobulin hinge region” refers to a naturally occurring upper and middle hinge amino acid sequences interposed between and connecting the CH1 and CH2 domains (for IgG, IgA, and IgD) or interposed between and connecting the CH1 and CH3 domains (for IgE and IgM) found in the heavy chain of an antibody.

An immunoglobulin hinge region may be an IgG, IgA, IgD, IgE, or IgM hinge region. An IgG hinge region may be an IgG1, IgG2, IgG3, or IgG4 hinge region. Sequences from IgG1, IgG2, IgG3, IgG4 or IgD can be used alone or in combination with all or a portion of a CH2 region; all or a portion of a CH3 region; or all or a portion of a CH2 region and all or a portion of a CH3 region.

Other examples of hinge regions used in fusion binding proteins described herein include the hinge region present in the extracellular regions of type 1 membrane proteins, such as CD8a, CD4, CD28 and CD7, which may be wild-type or variants thereof.

In particular embodiments, a spacer region includes a hinge region that includes a type II C-lectin interdomain (stalk) region or a cluster of differentiation (CD) molecule stalk region. A “stalk region” of a type II C-lectin or CD molecule refers to the portion of the extracellular domain of the type II C-lectin or CD molecule that is located between the C-type lectin-like domain (CTLD; e.g., similar to CTLD of natural killer cell receptors) and the hydrophobic portion (transmembrane domain). For example, the extracellular domain of human CD94 (GenBank Accession No. AAC50291.1) corresponds to amino acid residues 34-179, but the CTLD corresponds to amino acid residues 61-176, so the stalk region of the human CD94 molecule includes amino acid residues 34-60, which are located between the hydrophobic portion (transmembrane domain) and CTLD (see Boyington et al., Immunity 10:15, 1999; for descriptions of other stalk regions, see also Beavil et al., Proc. Nat'l. Acad. Sci. USA 89:153, 1992; and Figdor et al., Nat. Rev. Immunol. 2:11, 2002). These type II C-lectin or CD molecules may also have junction amino acids (described below) between the stalk region and the transmembrane region or the CTLD. In another example, the 233 amino acid human NKG2A protein (GenBank Accession No. P26715.1) has a hydrophobic portion (transmembrane domain) ranging from amino acids 71-93 and an extracellular domain ranging from amino acids 94-233. The CTLD includes amino acids 119-231 and the stalk region includes amino acids 99-116, which may be flanked by additional junction amino acids. Other type II C-lectin or CD molecules, as well as their extracellular ligand-binding domains, stalk regions, and CTLDs are known in the art (see, e.g., GenBank Accession Nos. NP 001993.2; AAH07037.1; NP 001773.1; AAL65234.1; CAA04925.1; for the sequences of human CD23, CD69, CD72, NKG2A, and NKG2D and their descriptions, respectively).

In particular embodiments, a spacer region is (GGGGS)n (SEQ ID NO: 53) wherein n is an integer including, 1, 2, 3, 4, 5, 6, 7, 8, 9, or more. In particular embodiments, the spacer region is (EAAAK)n (SEQ ID NO: 66) wherein n is an integer including 1, 2, 3, 4, 5, 6, 7, 8, 9, or more. Junction amino acids can be a short oligo- or protein linker, preferably between 2 and 9 amino acids (e.g., 2, 3, 4, 5, 6, 7, 8, or 9 amino acids) in length to form the linker. In particular embodiments, a glycine-serine doublet can be used as a suitable junction amino acid linker. In particular embodiments, a single amino acid, e.g., an alanine, a glycine, can be used as a suitable junction amino acid.

Linkers can be susceptible to cleavage (cleavable linker), such as, acid-induced cleavage, photo-induced cleavage, peptidase-induced cleavage, esterase-induced cleavage, and disulfide bond cleavage. Alternatively, linkers can be substantially resistant to cleavage (e.g., stable linker or non-cleavable linker). In some aspects, the linker is a procharged linker, a hydrophilic linker, or a dicarboxylic acid-based linker.

Anti-CD33 antibody conjugates are artificial molecules that include a molecule conjugated to a CD33 binding domain. Anti-CD33 antibody conjugates include anti-CD33 immunotoxins, ADCs, and radioisotope conjugates.

Anti-CD33 immunotoxins are artificial molecules that include a toxin linked to a CD33 binding domain. In particular embodiments, immunotoxins selectively deliver an effective dose of a cytotoxin to non-genetically modified CD33-expressing cells.

To prepare immunotoxins, linker-cytotoxin conjugates can be made by conventional methods analogous to those described by Doronina et al. (Bioconjugate Chem. 17: 114-124, 2006). Immunotoxins containing CD33 binding domains can be prepared by standard methods for cysteine conjugation, such as by methods analogous to that described in Hamblett et al., Clin. Cancer Res. 10:7063-7070, 2004; Doronina et al., Nat. Biotechnol. 21(7): 778-784, 2003; and Francisco et al., Blood 102:1458-1465, 2003.

Immunotoxins with multiple (e.g., four) cytotoxins per binding domain can be prepared by partial reduction of the binding domain with an excess of a reducing reagent such as dithiothreitol (DTT) or tris(2-carboxyethyl)phosphine (TCEP) at 37° C. for 30 min, then the buffer can be exchanged by elution through SEPHADEX G-25 resin with 1 mM DTPA (diethylene triamine penta-acetic acid) in Dulbecco's phosphate-buffered saline (DPBS). The eluent can be diluted with further DPBS, and the thiol concentration of the binding domain can be measured using 5,5′-dithiobis(2-nitrobenzoic acid) [Ellman's reagent]. An excess, for example 5-fold, of the linker-cytotoxin conjugate can be added at 4° C. for 1 hr, and the conjugation reaction can be quenched by addition of a substantial excess, for example 20-fold, of cysteine. The resulting immunotoxin mixture can be purified on SEPHADEX G-25 equilibrated in PBS to remove unreacted linker-cytotoxin conjugate, desalted if desired, and purified by size-exclusion chromatography. The resulting immunotoxin can then be sterile filtered, for example, through a 0.2 μm filter, and can be lyophilized if desired for storage.

Frequently used plant toxin drugs are divided into two classes: (1) holotoxins (or class II ribosome inactivating proteins), such as ricin, abrin, mistletoe lectin, and modeccin, and (2) hemitoxins (class I ribosome inactivating proteins), such as pokeweed antiviral protein (PAP), saporin, Bryodin 1, bouganin, and gelonin. Commonly used bacterial toxins include diphtheria toxin (DT) and Pseudomonas exotoxin (PE) (Kreitman, Curr Pharma Biotech 2:313-325, 2001). The toxin may also be an antibody or other peptide. Anti-CD33 ADCs include a CD33 binding domain linked to a cytotoxic drug that results in the bound cell's destruction. ADCs allow for the targeted delivery of a drug moiety to a selected cell, and, in particular embodiments intracellular accumulation therein, where systemic administration of unconjugated drugs may result in unacceptable levels of toxicity to normal cells (Polakis, Curr Op Pharmacol 5:382-387, 2005).

ADC can include targeted drugs which combine properties of both antibodies and cytotoxic drugs by targeting potent cytotoxic drugs to antigen-expressing cells (Teicher, B. A. (2009) Current Cancer Drug Targets 9:982-1004), thereby enhancing the therapeutic index by maximizing efficacy and minimizing off-target toxicity (Carter & Senter, (2008) The Cancer Jour. 14(3):154-169; Chari, (2008) Acc. Chem. Res. 41:98-107). See also Kamath & Iyer (Pharm Res. 32(11): 3470-3479, 2015), which describes considerations for the development of ADCs.

ADC compounds of the disclosure include those with anti-CD33 cell activity. In particular embodiments, the ADC compounds include a CD33 binding domain conjugated, i.e. covalently attached, to the drug moiety.

Examples of drugs useful to include within the ADC format include taxol, taxane, cytochalasin B, gramicidin D, ethidium bromide, emetine, mitomycin, etoposide, tenoposide, vincristine, vinblastine, colchicin, doxorubicin, daunorubicin, dihydroxy anthracinedione, mitoxantrone, mithramycin, actinomycin D, 1-dehydrotestosterone, glucocorticoids, procaine, tetracaine, lidocaine, propranolol, and puromycin and analogs or homologs thereof. Other appropriate toxins include, for example, CC-1065 and analogues thereof, the duocarmycins. Additional examples include maytansinoid (including monomethyl auristatin E [MMAE]; vedotin), dolastatin, auristatin, calicheamicin, pyrrolobenzodiazepine (PBD) dimer, indolino-benzodiazepine dimer, nemorubicin and its derivatives, PNU-159682, anthracycline, vinca alkaloid, trichothecene, camptothecin, elinafide, and stereoisomers, isosteres, analogs, and derivatives thereof that have cytotoxic activity.

The drug may be obtained from essentially any source; it may be synthetic or a natural product isolated from a selected source, e.g., a plant, bacterial, insect, mammalian or fungal source. The drug may also be a synthetically modified natural product or an analogue of a natural product.

Exemplary ADCs that target CD33 include GO (which includes the recombinant humanized IgG4 anti-CD33 hP67.6 antibody linked to the cytotoxic antitumor antibiotic calicheamicin; U.S. Pat. No. 5,773,001), lintuzumab (SGN-33; HuM195; Caron et al., Can. Res. 52:6761-6767, 1992), SGN-CD33A (the antibody portion of which is h2H12EC a.k.a h2H12d; see US 2013/0309223), and IMGN779.

Anti-CD33 antibody-radioisotope conjugates include a CD33 binding domain linked to a cytotoxic radioisotope for use in nuclear medicine. Nuclear medicine refers to the diagnosis and/or treatment of conditions by administering radioactive isotopes (radioisotopes or radionuclides) to a subject. Therapeutic nuclear medicine is often referred to as radiation therapy or radioimmunotherapy (RIT).

Examples of radioactive isotopes that can be conjugated to CD33 binding domains include iodine-131, indium-111, yttrium-90, and lutetium-177, as well as alpha-emitting radionuclides such as astatine-211 or bismuth-212, bismuth-213, or actinium-225. Methods for preparing radioimmunoconjugates are established in the art. Examples of radioimmunotoxins are commercially available, including Zevalin® (RIT Oncology, Seattle, Wash.), and similar methods can be used to prepare radioimmunotoxins using the binding domains of the disclosure.

Examples of radionuclides that are useful for radiation therapy include ²²⁵AC and ²²⁷Th. ²²⁵AC is a radionuclide with the half-life of ten days. As ²²⁵AC decays the daughter isotopes ²²¹Fr, ²¹³Bi, and ²⁰⁹Pb are formed. ²²⁷Th has a half-life of 19 days and forms the daughter isotope ²²³Ra.

Additional examples of useful radioisotopes include ²²⁸Ac, ¹¹¹Ag, ¹²⁴Am, ⁷⁴As, ²⁰⁹At, ¹⁹⁴Au, ¹²⁸Ba, ⁷Be, ²⁰⁶Bi, ²⁴⁶Bk, ²⁴⁶Bk, ⁷⁶Br, ¹¹C, ⁴⁷Ca, ²⁵⁴Cf, ²⁴²Cm, ⁵¹Cr, ⁶⁷Cu, ¹⁵³Dy, ¹⁵⁷Dy, ¹⁵⁹Dy, ¹⁶⁵Dy, ¹⁶⁶Dy, ¹⁷¹Er, ²⁵⁰Es, ²⁵⁴Es, ¹⁴⁷Eu, ¹⁵⁷Eu, ⁵²Fe, ⁵⁹Fe, ²⁵¹Fm, ²⁵²Fm, ²⁵³Fm, ⁶⁶Ga, ⁷²Ga, ¹⁴⁶Gd, ¹⁵³Gd, ⁶⁸Ge, ¹⁷⁰Hf, ¹⁷¹Hf, ¹⁹³Hg, ¹⁹³mHg, ¹⁶⁰mHo, ¹³⁰I, ¹³⁵I, ¹¹⁴mln, ¹⁸⁵Ir, ⁴²K, ⁴³K, ⁷⁶Kr, ⁷⁹Kr, ⁸¹mKr, ¹³²La, ²⁶²Lr, ¹⁶⁹Lu, ¹⁷⁴mLu, ¹⁷⁶mLu, ²⁵⁷Md, ²⁶⁰Md, ²⁸¹Mg, ⁵²¹Mn, ⁹⁰Mo, ²⁴Na, ⁹⁵Nb, ¹³⁸Nd, ⁵⁷Ni, ⁶⁶Ni, ²³⁴Np, ¹⁵O, ¹⁸²Os, ¹⁸⁹mOs, ¹⁹¹Os, ³²P, ²⁰¹Pb, ¹⁰¹Pd, ¹⁴³Pr, ¹⁹¹Pt, ²⁴³Pu, ²²⁵Ra, ⁸¹Rb, ¹⁸⁸Re, ¹⁰⁵Rh, ²¹¹Rn, ¹⁰³Ru, ³⁵S, ⁴⁴Sc, ⁷²Se, ¹⁵³Sm, ¹²⁵Sn, ⁹¹Sr, ¹⁷³Ta, ¹⁵⁴Tb, ¹²⁷Te, ²³⁴Th, ⁴⁵Ti, ¹⁶⁶Tm, ²³⁰U, ²³⁷U, ²⁴⁰U, ⁴⁸V, ¹⁷⁸W, ¹⁸¹W, ¹⁸⁸W, ¹²⁵Xe, ¹²⁷Xe, ¹³³Xe, ¹³³mXe, ¹³⁵Xe, ⁸⁵mY, ⁸⁶Y, ⁹³Y, ¹⁶⁹Yb, ¹⁷⁵Yb, ⁶⁵Zn, ⁷¹mZn, ⁸⁶Zr, ⁹⁵Zr, and/or ⁹⁷Zr.

In particular embodiments, CD33-targeting agents include bi- or trispecific immune cell engaging antibody constructs. An example of a bi- or trispecific immune cell engaging antibody construct includes those which bind both CD33 and an immune cell (e.g., T-cell) activating epitope, with the goal of bringing immune cells to CD33-expressing cells to destroy the CD33-expressing cells. See, for example, US 2008/0145362. Such constructs are referred to herein as immune-activating bi- or tri-specifics or I-ABTS). In particular embodiments, I-ABTS include AMG330, AMG673, and AMV-564. BiTEs® are one form of I-ABTS. Immune cells that can be targeted for localized activation by I-ABTS within the current disclosure include, for example, T-cells, natural killer (NK) cells, and macrophages which are discussed in more detail herein. Bispecific immune cell engaging antibody constructs, including I-ABTS utilize bispecific binding domains, such as bispecific antibodies to target CD33-expressing cells and immune cells. The binding domain that binds CD33 and the binding domain that binds and activates an immune cell may be joined through a linker, as described elsewhere herein.

T-cell activation can be mediated by two distinct signals: those that initiate antigen-dependent primary activation and provide a T-cell receptor like signal (primary cytoplasmic signaling sequences) and those that act in an antigen independent manner to provide a secondary or co-stimulatory signal (secondary cytoplasmic signaling sequences). I-ABTS disclosed herein can target any T-cell activating epitope that upon binding induces T-cell activation. Examples of such T-cell activating epitopes are on T-cell markers including CD2, CD3, CD7, CD27, CD28, CD30, CD40, CD83, 4-1BB (CD 137), OX40, lymphocyte function-associated antigen-1 (LFA-1), LIGHT, NKG2C, and B7-H3.

Several different subsets of T-cells have been discovered, each with a distinct function. For example, a majority of T-cells have a TCR existing as a complex of several proteins. The actual T-cell receptor is composed of two separate peptide chains, which are produced from the independent T-cell receptor α and β (TCRα and TCRρ) genes and are called α- and β-TCR chains.

CD3 is a primary signal transduction element of T-cell receptors. CD3 is composed of a group of invariant proteins called gamma (γ), delta (δ), epsilon (ε), zeta (ζ) and eta (η) chains. The γ, δ, and ε chains are structurally-related, each containing an Ig-like extracellular constant domain followed by a transmembrane region and a cytoplasmic domain of more than 40 amino acids. The ζ and η chains have a distinctly different structure: both have a very short extracellular region of only 9 amino acids, a transmembrane region and a long cytoplasmic tail including 113 and 115 amino acids in the and q chains, respectively. The invariant protein chains in the CD3 complex associate to form noncovalent heterodimers of the ε chain with a γ chain (εγ) or with a δ chain (εδ) or of the ζ and η chain (ζη), or a disulfide-linked homodimer of two ζ chains (ζζ). 90% of the CD3 complex incorporate the ζζ homodimer.

The cytoplasmic regions of the CD3 chains include a motif designated the immunoreceptor tyrosine-based activation motif (ITAM). This motif is found in a number of other receptors including the Ig-α/Ig-β heterodimer of the B-cell receptor complex and Fc receptors for IgE and IgG. The ITAM sites associate with cytoplasmic tyrosine kinases and participate in signal transduction following TCR-mediated triggering. In CD3, the γ, δ and ε chains each contain a single copy of ITAM, whereas the ζ and η chains harbor three ITAMs in their long cytoplasmic regions. Indeed, the ζ and η chains have been ascribed a major role in T-cell activation signal transduction pathways.

In particular embodiments, the CD3 binding domain (e.g., scFv) of an I-ABTS is derived from the OKT3 antibody (also utilized in blinatumomab). The OKT3 antibody is described in detail in U.S. Pat. No. 5,929,212. It includes a variable light chain including a CDRL1 sequence including SASSSVSYMN (SEQ ID NO: 67), a CDRL2 sequence including RWIYDTSKLAS (SEQ ID NO: 68), and a CDRL3 sequence including QQWSSNPFT (SEQ ID NO: 69). In particular embodiments, the CD3 T-cell activating epitope binding domain is a human or humanized binding domain (e.g., scFv) including a variable heavy chain including a CDRH1 sequence including KASGYTFTRYTMH (SEQ ID NO: 70), a CDRH2 sequence including INPSRGYTNYNQKFKD (SEQ ID NO: 71), and a CDRH3 sequence including YYDDHYCLDY (SEQ ID NO: 72).

The following sequence is an scFv derived from OKT3 which retains the capacity to bind CD3:

(SEQ ID NO: 73)
QVQLQQSGAELARPGASVKMSCKASGYTFTRYTMHWVKQRPGQGLEWIG
YINPSRGYTNYNQKFKDKATLTTDKSSSTAYMQLSSLTSEDSAVYYCAR
YYDDHYCLDYWGQGTTLTVSSSGGGGSGGGGSGGGGSQIVLTQSPAIMS
ASPGEKVTMTCSASSSVSYMNWYQQKSGTSPKRWIYDTSKLASGVPAHF
RGSGSGTSYSLTISGMEAEDAATYYCQQWSSNPFTFGSGTKLEINR.

It may also be used as a CD3 binding domain.

In particular embodiments, the CD3 T-cell activating epitope binding domain is a human or humanized binding domain (e.g., scFv) including a variable light chain including a CDRL1 sequence including QSLVHNNGNTY (SEQ ID NO: 74), a CDRL2 sequence including KVS, and a CDRL3 sequence including GQGTQYPFT (SEQ ID NO: 75). In particular embodiments, the CD3 T-cell activating epitope binding domain is a human or humanized binding domain (e.g., scFv) including a variable heavy chain including a CDRH1 sequence including GFTFTKAW (SEQ ID NO: 76), a CDRH2 sequence including IKDKSNSYAT (SEQ ID NO: 77), and a CDRH3 sequence including RGVYYALSPFDY (SEQ ID NO: 78). These reflect CDR sequences of the 20G6-F3 antibody.

In particular embodiments, the CD3 T-cell activating epitope binding domain is a human or humanized binding domain (e.g., scFv) including a variable light chain including a CDRL1 sequence including QSLVHDNGNTY (SEQ ID NO: 79), a CDRL2 sequence including KVS, and a CDRL3 sequence including GQGTQYPFT (SEQ ID NO: 75). In particular embodiments, the CD3 T-cell activating epitope binding domain is a human or humanized binding domain (e.g., scFv) including a variable heavy chain including a CDRH1 sequence including GFTFSNAW (SEQ ID NO: 81), a CDRH2 sequence including IKARSNNYAT (SEQ ID NO: 82), and a CDRH3 sequence including RGTYYASKPFDY (SEQ ID NO: 83). These reflect CDR sequences of the 4B4-D7 antibody.

In particular embodiments, the CD3 T-cell activating epitope binding domain is a human or humanized binding domain (e.g., scFv) including a variable light chain including a CDRL1 sequence including QSLEHNNGNTY (SEQ ID NO: 84), a CDRL2 sequence including KVS, and a CDRL3 sequence including GQGTQYPFT (SEQ ID NO: 75). In particular embodiments, the CD3 T-cell activating epitope binding domain is a human or humanized binding domain (e.g., scFv) including a variable heavy chain including a CDRH1 sequence including GFTFSNAW (SEQ ID NO: 81), a CDRH2 sequence including IKDKSNNYAT (SEQ ID NO: 87), and a CDRH3 sequence including RYVHYGIGYAMDA (SEQ ID NO: 88). These reflect CDR sequences of the 4E7-C9 antibody.

In particular embodiments, the CD3 T-cell activating epitope binding domain is a human or humanized binding domain (e.g., scFv) including a variable light chain including a CDRL1 sequence including QSLVHTNGNTY (SEQ ID NO: 89), a CDRL2 sequence including KVS, and a CDRL3 sequence including GQGTHYPFT (SEQ ID NO: 90). In particular embodiments, the CD3 T-cell activating epitope binding domain is a human or humanized binding domain (e.g., scFv) including a variable heavy chain including a CDRH1 sequence including GFTFTNAW (SEQ ID NO: 91), a CDRH2 sequence including KDKSNNYAT (SEQ ID NO: 92), and a CDRH3 sequence including RYVHYRFAYALDA (SEQ ID NO: 93). These reflect CDR sequences of the 18F5-H10 antibody.

Additional examples of anti-CD3 antibodies, binding domains, and CDRs can be found in WO2016/116626. TR66 may also be used. WO 2015/036583 describes a bispecific antibody construct that binds to CD33 and CD3.

CD28 is a surface glycoprotein present on 80% of peripheral T-cells in humans and is present on both resting and activated T-cells. CD28 binds to B7-1 (CD80) and B7-2 (CD86) and is the most potent of the known co-stimulatory molecules (June et al., Immunol. Today 15:321, 1994; Linsley et al., Ann. Rev. Immunol. 11:191, 1993). In particular embodiments, the CD28 binding domain (e.g., scFv) is derived from CD80, CD86 or the 9D7 antibody. Additional antibodies that bind CD28 include 9.3, KOLT-2, 15E8, 248.23.2, and EX5.3D10. Further, 1YJD provides a crystal structure of human CD28 in complex with the Fab fragment of a mitogenic antibody (5.11A1). In particular embodiments, antibodies that do not compete with 9D7 are selected.

In particular embodiments, a CD28 binding domain is derived from TGN1412. In particular embodiments, the variable heavy chain of TGN1412 includes:

(SEQ ID NO: 94)
QVQLVQSGAEVKKPGASVKVSCKASGYTFTSYYIHWVRQAPGQGLEWIGC
IYPGNVNTNYNEKFKDRATLTVDTSISTAYMELSRLRSDDTAVYFCTRSH
YGLDWNFDVWGQGTTVTVSS

and the variable light chain of TGN1412 includes:

(SEQ ID NO: 95)
DIQMTQSPSSLSASVGDRVTITCHASQNIYVWLNWYQQKPGKAPKLLIY
KASNLHTGVPSRFSGSGSGTDFTLTISSLQPEDFATYYCQQGQTYPYTF
GGGTKVEIK.

In particular embodiments, the CD28 binding domain includes a variable light chain including a CDRL1 sequence including HASQNIYVWLN (SEQ ID NO: 96), CDRL2 sequence including KASNLHT (SEQ ID NO: 97), and CDRL3 sequence including QQGQTYPYT (SEQ ID NO: 98), a variable heavy chain including a CDRH1 sequence including GYTFTSYYIH (SEQ ID NO: 99), a CDRH2 sequence including CIYPGNVNTNYNEK (SEQ ID NO: 100), and a CDRH3 sequence including SHYGLDWNFDV (SEQ ID NO: 101).

In particular embodiments, the CD28 binding domain including a variable light chain including a CDRL1 sequence including HASQNIYVWLN (SEQ ID NO: 96), a CDRL2 sequence including KASNLHT (SEQ ID NO: 97), and a CDRL3 sequence including QQGQTYPYT (SEQ ID NO: 98) and a variable heavy chain including a CDRH1 sequence including SYYIH (SEQ ID NO: 105), a CDRH2 sequence including CIYPGNVNTNYNEKFKD (SEQ ID NO: 106), and a CDRH3 sequence including SHYGLDWNFDV (SEQ ID NO: 101).

In particular embodiments, activated T-cells express 4-1BB (CD137). In particular embodiments, the 4-1 BB binding domain includes a variable light chain including a CDRL1 sequence including RASQSVS (SEQ ID NO: 108), a CDRL2 sequence including ASNRAT (SEQ ID NO: 109), and a CDRL3 sequence including QRSNWPPALT (SEQ ID NO: 110) and a variable heavy chain including a CDRH1 sequence including YYWS (SEQ ID NO: 111), a CDRH2 sequence including INH, and a CDRH3 sequence including YGPGNYDWYFDL (SEQ ID NO: 112).

In particular embodiments, the 4-1BB binding domain includes a variable light chain including a CDRL1 sequence including SGDNIGDQYAH (SEQ ID NO: 113), a CDRL2 sequence including QDKNRPS (SEQ ID NO: 114), and a CDRL3 sequence including ATYTGFGSLAV (SEQ ID NO: 115) and a variable heavy chain including a CDRH1 sequence including GYSFSTYWIS (SEQ ID NO: 116), a CDRH2 sequence including KIYPGDSYTNYSPS (SEQ ID NO: 117) and a CDRH3 sequence including GYGIFDY (SEQ ID NO: 118).

Particular embodiments disclosed herein include immune cell binding domains that bind epitopes on CD8. In particular embodiments, the CD8 binding domain (e.g., scFv) is derived from the OKT8 antibody. For example, in particular embodiments, the CD8 T-cell activating epitope binding domain is a human or humanized binding domain (e.g., scFv) including a variable light chain including a CDRL1 sequence including RTSRSISQYLA (SEQ ID NO: 119), a CDRL2 sequence including SGSTLQS (SEQ ID NO: 120), and a CDRL3 sequence including QQHNENPLT (SEQ ID NO: 121). In particular embodiments, the CD8 T-cell activating epitope binding domain is a human or humanized binding domain (e.g., scFv) including a variable heavy chain including a CDRH1 sequence including GFNIKD (SEQ ID NO: 122), a CDRH2 sequence including RIDPANDNT (SEQ ID NO: 123), and a CDRH3 sequence including GYGYYVFDH (SEQ ID NO: 124). These reflect CDR sequences of the OKT8 antibody.

In particular embodiments, an immune cell binding domain is a scTCR including V_{α/β} and C_{α/β} chains (e.g., V_{α}-C_{α}, V_{β}-C_{β}, V_{α}-V_{β}) or including V_{α}-C_{α}, V_{β}-C_{β}, V_{α}-V_{β} pair specific for a target epitope of interest. In particular embodiments, T-cell activating epitope binding domains can be derived from or based on a Vα, Vβ, Cα, or Cβ of a known TCR (e.g., a high-affinity TCR).

In particular embodiments, natural killer cells (also known as NK cells, K cells, and killer cells) are activated in response to interferons or macrophage-derived cytokines. They serve to contain viral infections while the adaptive immune response is generating antigen-specific cytotoxic T cells that can clear the infection. NK cells express CD8, CD16 and CD56 but do not express CD3.

In particular embodiments NK cells are targeted for localized activation by I-ABTS. NK cells can induce apoptosis or cell lysis by releasing granules that disrupt cellular membranes and can secrete cytokines to recruit other immune cells.

Examples of activating proteins expressed on the surface of NK cells include NKG2D, CD8, CD16, KIR2DL4, KIR2DS1, KIR2DS2, KIR3DS1, NKG2C, NKG2E, NKG2D, and several members of the natural cytotoxicity receptor (NCR) family. Examples of NCRs that activate NK cells upon ligand binding include NKp30, NKp44, NKp46, NKp80, and DNAM-1.

Examples of commercially available antibodies that bind to an NK cell receptor and induce and/or enhance activation of NK cells include: 5C6 and 1 D11, which bind and activate NKG2D (available from BioLegend® San Diego, Calif.); mAb 33, which binds and activates KIR2DL4 (available from BioLegend®); P44-8, which binds and activates NKp44 (available from BioLegend®); SK1, which binds and activates CD8; and 3G8 which binds and activates CD16.

In particular embodiments, the I-ABTS can bind to and block an NK cell inhibitory receptor to enhance NK cell activation. Examples of NK cell inhibitory receptors that can be bound and blocked include KIR2DL1, KIR2DL2/3, KIR3DL1, NKG2A, and KLRG1. In particular embodiments, a binding domain that binds and blocks the NK cell inhibitory receptors KIR2DL1 and KIR2DL2/3 includes a variable light chain region of the sequence:

(SEQ ID NO: 125)
EIVLTQSPVTLSLSPGERATLSCRASQSVSSYLAWYQQKPGQAPRLLIY
DASNRATGIPARFSGSGSGTDFTLTISSLEPEDFAVYYCQQRSNWMYTF
GQGTKLEIKRT

and a variable heavy chain region of the sequence:

(SEQ ID NO: 126)
QVQLVQSGAEVKKPGSSVKVSCKASGGTFSFYAISWVRQAPGQGLEWMG
GFIPIFGAANYAQKFQGRVTITADESTSTAYMELSSLRSDDTAVYYCAR
IPSGSYYYDYDMDVWGQGTTVTVSS.

Additional NK cell activating antibodies are described in WO2005/0003172 and U.S. Pat. No. 9,415,104.

Macrophages (and their precursors, monocytes) reside in every tissue of the body (in certain instances as microglia, Kupffer cells and osteoclasts) where they can engulf apoptotic cells, pathogens and other non-self-components.

The I-ABTS can be designed to bind to a protein expressed on the surface of macrophages. Examples of activating proteins expressed on the surface of macrophages (and their precursors, monocytes) include CD11b, CD11c, CD64, CD68, CD119, CD163, CD206, CD209, F4/80, IFGR2, Toll-like receptors (TLRs) 1-9, IL-4Ra, and MARCO. Commercially available antibodies that bind to proteins expressed on the surface of macrophages include M1/70, which binds and activates CD11 b (available from BioLegend); KP1, which binds and activates CD68 (available from ABCAM, Cambridge, United Kingdom); and ab87099, which binds and activates CD163 (available from ABCAM).

In particular embodiments, anti-CD33 tri-specific antibodies are artificial proteins that simultaneously bind to three different types of antigens, wherein at least one of the antigens is CD33. Tri-specific antibodies are described in, for example, WO2016/105450, WO 2010/028796; WO 2009/007124; WO 2002/083738; US 2002/0051780; and WO 2000/018806.

When CD33-targeting agents are based on antibodies, binding domains, or similar proteins derived therefrom, modifications that provide different administration benefits can be useful. Exemplary administration benefits can include (1) reduced susceptibility to proteolysis, (2) reduced susceptibility to oxidation, (3) altered binding affinity for forming protein complexes, (4) altered binding affinities, (5) reduced immunogenicity; and/or (6) extended half-live. While the present disclosure describes these modifications in terms of their application to antibodies, when applicable to another particular anti-CD33 binding domain format (e.g., an scFv, bispecific antibodies), the modifications can also be applied to these other formats.

In particular embodiments antibodies can be mutated to increase the half-life of the antibodies in serum. M428L/N434S is a pair of mutations that increase the half-life of antibodies in serum, as described in Zalevsky et al., Nature Biotechnology 28, 157-159, 2010.

In particular embodiments antibodies can be mutated to increase their affinity for Fc receptors. Exemplary mutations that increase the affinity for Fc receptors include: G236A/S239D/A330L/I332E (GASDALIE). Smith et al., Proceedings of the National Academy of Sciences of the United States of America, 109(16), 6181-6186, 2012. In particular embodiments, an antibody variant includes an Fc region with one or more amino acid substitutions which improve ADCC, e.g., substitutions at positions 298, 333, and/or 334 of the Fc region (EU numbering of residues). In particular embodiments, alterations are made in the Fc region that result in altered Clq binding and/or Complement Dependent Cytotoxicity (CDC), e.g., as described in U.S. Pat. No. 6,194,551, WO 99/51642, and Idusogie et al., J. Immunol. 164: 4178-4184, 2000.

Antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g. complex, hybrid and high mannose structures) as measured by MALDI-TOF mass spectrometry, as described in WO 2008/077546, for example. Asn297 refers to the asparagine residue located at position 297 in the Fc region (Eu numbering of Fc region residues); however, Asn297 may also be located ±3 amino acids upstream or downstream of position 297, i.e., between positions 294 and 300, due to minor sequence variations in antibodies. Such fucosylation variants may have improved ADCC function. See, e.g., WO2000/61739; WO 2001/29246; WO2002/031140; US2002/0164328; WO2003/085119; WO2003/084570; US2003/0115614; US2003/0157108; US2004/0093621; US2004/0110704; US2004/0132140; US2004/0110282; US2004/0109865; WO2005/035586; WO2005/035778; WO2005/053742; Okazaki et al. J. Mol. Biol. 336:1239-1249 (2004); and Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545, 1986, and knockout cell lines, such as alpha-1,6-fucosyltransferase gene, FUT8, knockout CHO cells (see, e.g., Yamane-Ohnuki et al., Biotech. Bioeng. 87: 614, 2004; Kanda et al., Biotechnol. Bioeng., 94(4):680-688, 2006; and WO2003/085107).

In particular embodiments, modified antibodies include those wherein one or more amino acids have been replaced with a non-amino acid component, or where the amino acid has been conjugated to a functional group or a functional group has been otherwise associated with an amino acid. The modified amino acid may be, e.g., a glycosylated amino acid, a PEGylated amino acid, a farnesylated amino acid, an acetylated amino acid, a biotinylated amino acid, an amino acid conjugated to a lipid moiety, or an amino acid conjugated to an organic derivatizing agent. Amino acid(s) can be modified, for example, co-translationally or post-translationally during recombinant production (e.g., N-linked glycosylation at N-X-S/T motifs during expression in mammalian cells) or modified by synthetic means. The modified amino acid can be within the sequence or at the terminal end of a sequence. Modifications also include nitrited constructs.

PEGylation particularly is a process by which polyethylene glycol (PEG) polymer chains are covalently conjugated to other molecules such as proteins. Several methods of PEGylating proteins have been reported in the literature. For example, N-hydroxy succinimide (NHS)-PEG was used to PEGylate the free amine groups of lysine residues and N-terminus of proteins; PEGs bearing aldehyde groups have been used to PEGylate the amino-termini of proteins in the presence of a reducing reagent; PEGs with maleimide functional groups have been used for selectively PEGylating the free thiol groups of cysteine residues in proteins; and site-specific PEGylation of acetyl-phenylalanine residues can be performed.

Covalent attachment of proteins to PEG has proven to be a useful method to increase the half-lives of proteins in the body (Abuchowski, A. et al., Cancer Biochem. Biophys., 1984, 7:175-186; Hershfield, M. S. et al., N. Engl. J. Medicine, 1987, 316:589-596; and Meyers, F. J. et al., Clin. Pharmacol. Ther., 49:307-313, 1991). The attachment of PEG to proteins not only protects the molecules against enzymatic degradation, but also reduces their clearance rate from the body. The size of PEG attached to a protein has significant impact on the half-life of the protein. The ability of PEGylation to decrease clearance is generally not a function of how many PEG groups are attached to the protein, but the overall molecular weight of the altered protein. Usually the larger the PEG is, the longer the in vivo half-life of the attached protein. In addition, PEGylation can also decrease protein aggregation (Suzuki et al., Biochem. Bioph. Acta 788:248, 1984), alter protein immunogenicity (Abuchowski et al., J. Biol. Chem. 252: 3582, 1977), and increase protein solubility as described, for example, in PCT Publication No. WO 92/16221).

Several sizes of PEGs are commercially available (Nektar Advanced PEGylation Catalog 2005-2006; and NOF DDS Catalogue Ver 7.1), which are suitable for producing proteins with targeted circulating half-lives. A variety of active PEGs have been used including mPEG succinimidyl succinate, mPEG succinimidyl carbonate, and PEG aldehydes, such as mPEG-propionaldehyde.

CD33-targeting agents also include immune cells expressing CARs or TCRs that specifically bind CD33. Methods to genetically modify cells to express an exogenous gene are described above in section (IV).

CAR refer to proteins including several distinct subcomponents. The subcomponents include at least an extracellular component, a transmembrane domain, and an intracellular component. Within the current disclosure, the extracellular component includes a binding domain that binds CD33. When the binding domain binds CD33, the intracellular component signals the immune cell to destroy the bound cell. Binding domains that specifically bind CD33 are described above.

In various embodiments, intracellular or otherwise the cytoplasmic signaling components of a CAR are responsible for activation of the cell in which the CAR is expressed. The term “intracellular signaling components” or “intracellular components” is thus meant to include any portion of the intracellular domain sufficient to transduce an activation signal. Intracellular components of expressed CAR can include effector domains. An effector domain is an intracellular portion of a fusion protein or receptor that can directly or indirectly promote a biological or physiological response in a cell when receiving the appropriate signal. In certain embodiments, an effector domain is part of a protein or protein complex that receives a signal when bound, or it binds directly to a target molecule, which triggers a signal from the effector domain. An effector domain may directly promote a cellular response when it contains one or more signaling domains or motifs, such as an immunoreceptor tyrosine-based activation motif (ITAM). In other embodiments, an effector domain will indirectly promote a cellular response by associating with one or more other proteins that directly promote a cellular response, such as co-stimulatory domains.

Effector domains can provide for activation of at least one function of a modified cell upon binding to the cellular marker expressed by a CD33-expressing cell. Activation of the modified cell can include one or more of differentiation, proliferation and/or activation or other effector functions. In particular embodiments, an effector domain can include an intracellular signaling component including a T cell receptor and a co-stimulatory domain which can include the cytoplasmic sequence from a co-receptor or co-stimulatory molecule.

An effector domain can include one, two, three or more receptor signaling domains, intracellular signaling components (e.g., cytoplasmic signaling sequences), co-stimulatory domains, or combinations thereof. Exemplary effector domains include signaling and stimulatory domains selected from: 4-1BB (CD137), CARD11, CD3γ, CD3δ, CD3ε, CD3ζ, CD27, CD28, CD79A, CD79B, DAP10, FcRα, FcRβ (FcεR1b), FcRγ, Fyn, HVEM (LIGHTR), ICOS, LAGS, LAT, Lck, LRP, NKG2D, NOTCH1, pTα, PTCH2, OX40, ROR2, Ryk, SLAMF1, Slp76, TCRα, TCRβ, TRIM, Wnt, Zap70, or any combination thereof. In particular embodiments, exemplary effector domains include signaling and co-stimulatory domains selected from: CD86, FcγRIIa, DAP12, CD30, CD40, PD-1, lymphocyte function-associated antigen-1 (LFA-1), CD2, CD7, LIGHT, NKG2C, B7-H3, a ligand that specifically binds with CD83, CDS, ICAM-1, GITR, BAFFR, SLAMF7, NKp80 (KLRF1), CD127, CD160, CD19, CD4, CD8α, CD8β, IL2Rβ, IL2Rγ, IL7Rα, ITGA4, VLA1, CD49a, IA4, CD49D, ITGA6, VLA-6, CD49f, ITGAD, CD11d, ITGAE, CD103, ITGAL, CD11a, ITGAM, CD11b, ITGAX, CD11c, ITGB1, CD29, ITGB2, CD18, ITGB7, TNFR2, TRANCE/RANKL, DNAM1 (CD226), SLAMF4 (CD244, 2B4), CD84, CD96 (Tactile), CEACAM1, CRTAM, Ly9 (CD229), PSGL1, CD100 (SEMA4D), CD69, SLAMF6 (NTB-A, Ly108), SLAM (CD150, IPO-3), BLAME (SLAMF8), SELPLG (CD162), LTBR, GADS, PAG/Cbp, NKp44, NKp30, or NKp46.

Intracellular signaling component sequences that act in a stimulatory manner may include iTAMs. Examples of iTAMs including primary cytoplasmic signaling sequences include those derived from CD3γ, CD3δ, CD3ε, CD3ζ, CD5, CD22, CD66d, CD79a, CD79b, and common FcRγ (FCER1 G), FcγRIIa, FcRβ (Fcε Rib), DAP10, and DAP12. In particular embodiments, variants of CD3 retain at least one, two, three, or all ITAM regions.

Additional examples of intracellular signaling components include the cytoplasmic sequences of the CD3ζ chain, and/or co- receptors that act in concert to initiate signal transduction following binding domain engagement.

A co-stimulatory domain is a domain whose activation can be required for an efficient lymphocyte response to cellular marker binding. Some molecules are interchangeable as intracellular signaling components or co-stimulatory domains. Examples of costimulatory domains include CD27, CD28, 4-1BB (CD 137), OX40, CD30, CD40, PD-1, ICOS, lymphocyte function-associated antigen-1 (LFA-1), CD2, CD7, LIGHT, NKG2C, B7-H3, and a ligand that specifically binds with CD83. Further examples of such co-stimulatory domain molecules include CDS, ICAM-1, GITR, BAFFR, HVEM (LIGHTR), SLAMF7, NKp80 (KLRF1), NKp44, NKp30, NKp46, CD160, CD19, CD4, CD8a, CD8β, IL2Rβ, IL2Rγ, IL7Rα, ITGA4, VLA1, CD49a, ITGA4, IA4, CD49D, ITGA6, VLA-6, CD49f, ITGAD, CDlld, ITGAE, CD103, ITGAL, CDlla, ITGAM, CDI Ib, ITGAX, CDllc, ITGBI, CD29, ITGB2, CD18, ITGB7, TNFR2, TRANCE/RANKL, DNAM1 (CD226), SLAMF4 (CD244, 2B4), CD84, CD96 (Tactile), NKG2D, CEACAM1, CRTAM, Ly9 (CD229), PSGL1, CD100 (SEMA4D), CD69, SLAMF6 (NTB-A, LyI08), SLAM (SLAMF1, CD150, IPO-3), BLAME (SLAMF8), SELPLG (CD162), LTBR, LAT, GADS, SLP-76, PAG/Cbp, and CD19a.

In particular embodiments, the intracellular signaling component includes (i) all or a portion of the signaling domain of CD3, (ii) all or a portion of the signaling domain of 4-1BB, or (iii) all or a portion of the signaling domain of CD3 and 4-1BB.

Intracellular components may also include one or more of a protein of a Wnt signaling pathway (e.g., LRP, Ryk, or ROR2), NOTCH signaling pathway (e.g., NOTCH1, NOTCH2, NOTCH3, or NOTCH4), Hedgehog signaling pathway (e.g., PTCH or SMO), receptor tyrosine kinases (RTKs) (e.g., epidermal growth factor (EGF) receptor family, fibroblast growth factor (FGF) receptor family, hepatocyte growth factor (HGF) receptor family, insulin receptor (IR) family, platelet-derived growth factor (PDGF) receptor family, vascular endothelial growth factor (VEGF) receptor family, tropomycin receptor kinase (Trk) receptor family, ephrin (Eph) receptor family, AXL receptor family, leukocyte tyrosine kinase (LTK) receptor family, tyrosine kinase with immunoglobulin-like and EGF-like domains 1 (TIE) receptor family, receptor tyrosine kinase-like orphan (ROR) receptor family, discoidin domain (DDR) receptor family, rearranged during transfection (RET) receptor family, tyrosine-protein kinase-like (PTK7) receptor family, related to receptor tyrosine kinase (RYK) receptor family, or muscle specific kinase (MuSK) receptor family); G-protein-coupled receptors, GPCRs (Frizzled or Smoothened); serine/threonine kinase receptors (BMPR or TGFR); or cytokine receptors (ID R, IL2R, IL7R, or IL15R).

In particular embodiments, transmembrane domains within a CAR molecule connect the extracellular component and intracellular component through the cell membrane. The transmembrane domain can anchor the expressed molecule in the modified cell's membrane.

A transmembrane domain can be derived either from a natural and/or a synthetic source. When the source is natural, the transmembrane domain can be derived from any membrane-bound or transmembrane protein. Transmembrane domains can include at least the transmembrane region(s) of the α, β or ζ chain of a T-cell receptor, CD28, CD27, CD3 epsilon, CD45, CD4, CD5, CD8, CD9, CD16, CD22; CD33, CD37, CD64, CD80, CD86, CD134, CD137 and CD154. In particular embodiments, a transmembrane domain may include at least the transmembrane region(s) of, e.g., KIRDS2, OX40, CD2, CD27, LFA-1 (CD 11a, CD18), ICOS (CD278), 4-1BB (CD137), GITR, CD40, BAFFR, HVEM (LIGHTR), SLAMF7, NKp80 (KLRF1), NKp44, NKp30, NKp46, CD160, CD19, IL2Rβ, IL2Rγ, IL7R a, ITGA1, VLA1, CD49a, ITGA4, IA4, CD49D, ITGA6, VLA-6, CD49f, ITGAD, CDI Id, ITGAE, CD103, ITGAL, CDI la, ITGAM, CDI Ib, ITGAX, CDI Ic, ITGB1, CD29, ITGB2, CD18, ITGB7, TNFR2, DNAM1(CD226), SLAMF4 (CD244, 2B4), CD84, CD96 (Tactile), CEACAM1, CRT AM, Ly9(CD229), PSGL1, CD100 (SEMA4D), SLAMF6 (NTB-A, Lyl08), SLAM (SLAMF1, CD150, IPO-3), BLAME (SLAMF8), SELPLG (CD162), LTBR, PAG/Cbp, NKG2D, or NKG2C. In particular embodiments, a variety of human hinges can be employed as well including the human Ig (immunoglobulin) hinge (e.g., an IgG4 hinge, an IgD hinge), a GS linker (e.g., a Gly-Ser linker such as those described herein), a KIR2DS2 hinge or a CD8a hinge.

In particular embodiments, a transmembrane domain has a three-dimensional structure that is thermodynamically stable in a cell membrane, and generally ranges in length from 15 to 30 amino acids. The structure of a transmembrane domain can include an α helix, a β barrel, a β sheet, a β helix, or any combination thereof.

A transmembrane domain can include one or more additional amino acids adjacent to the transmembrane region, e.g., one or more amino acid within the extracellular region of the CAR (e.g., up to 15 amino acids of the extracellular region) and/or one or more additional amino acids within the intracellular region of the CAR (e.g., up to 15 amino acids of the intracellular components). In one aspect, the transmembrane domain is from the same protein that the signaling domain, co-stimulatory domain or the hinge domain is derived from. In another aspect, the transmembrane domain is not derived from the same protein that any other domain of the CAR is derived from. In some instances, the transmembrane domain can be selected or modified by amino acid substitution to avoid and/or reduce binding of such domains to the transmembrane domains of the same or different surface membrane proteins to minimize interactions with other unintended members of the receptor complex. In one aspect, the transmembrane domain is capable of homodimerization with another CAR on the cell surface of a CAR-expressing cell. In a different aspect, the amino acid sequence of the transmembrane domain may be modified or substituted so as to minimize interactions with the binding domains of the native binding partner present in the same CAR-expressing cell.

CARs and TCRs expressed by genetically modified immune cells often additionally include spacer regions. Spacer regions can position the binding domain away from the immune cell (e.g., T cell) surface to enable proper cell/cell contact, antigen binding and activation (Patel et al., Gene Therapy 6: 412-419, 1999). As indicated, an extracellular spacer region of a fusion binding protein is generally located between a hydrophobic portion or transmembrane domain and the extracellular binding domain, and the spacer region length may be varied to maximize antigen recognition (e.g., tumor recognition) based on the selected target molecule, selected binding epitope, or antigen-binding domain size and affinity (see, e.g., Guest et al., J. Immunother. 28:203-11, 2005; WO 2014/031687). Junction amino acids can be a linker which can be used to connect the sequences of CAR domains when the distance provided by a spacer is not needed and/or wanted. Junction amino acids are short amino acid sequences that can be used to connect co-stimulatory intracellular signaling components. In particular embodiments, junction amino acids are 9 amino acids or less.

Exemplary methods to produce CD33 CAR T-cells are described in WO2018/US34743.

In particular embodiments, cells genetically modified to express a CAR or TCR can additionally express one or more tag cassettes, transduction markers, and/or suicide switches. In some embodiments, the transduction marker and/or suicide switch is within the same construct but is expressed as a separate molecule on the cell surface. Tag cassettes and transduction markers can be used to activate, promote proliferation of, detect, enrich for, isolate, track, deplete and/or eliminate cells genetically modified to express a CAR or TCR in vitro, in vivo and/or ex vivo. “Tag cassette” refers to a unique synthetic peptide sequence affixed to, fused to, or that is part of a CD33-targeting agent, to which a cognate binding molecule (e.g., ligand, antibody, or other binding partner) is capable of specifically binding where the binding property can be used to activate, promote proliferation of, detect, enrich for, isolate, track, deplete and/or eliminate the tagged protein and/or cells expressing the tagged protein. Transduction markers can serve the same purposes but are derived from naturally occurring molecules and are often expressed using a skipping element that separates the transduction marker from the rest of the CD33-targeting agent.

Tag cassettes that bind cognate binding molecules include, for example, His tag (SEQ ID NO: 127), Flag tag (SEQ ID NO: 128), Xpress tag (SEQ ID NO: 129), Avi tag (SEQ ID NO: 130), Calmodulin tag (SEQ ID NO: 131), Polyglutamate tag, HA tag (SEQ ID NO: 132), Myc tag (SEQ ID NO: 133), Softag 1 (SEQ ID NO: 134), Softag 3 (SEQ ID NO: 135), and V5 tag (SEQ ID NO: 136).

Conjugate binding molecules that specifically bind tag cassette sequences disclosed herein are commercially available. For example, His tag antibodies are commercially available from suppliers including Life Technologies, Pierce Antibodies, and GenScript. Flag tag antibodies are commercially available from suppliers including Pierce Antibodies, GenScript, and Sigma-Aldrich. Xpress tag antibodies are commercially available from suppliers including Pierce Antibodies, Life Technologies and GenScript. Avi tag antibodies are commercially available from suppliers including Pierce Antibodies, IsBio, and Genecopoeia. Calmodulin tag antibodies are commercially available from suppliers including Santa Cruz Biotechnology, Abcam, and Pierce Antibodies. HA tag antibodies are commercially available from suppliers including Pierce Antibodies, Cell Signaling Technology and Abcam. Myc tag antibodies are commercially available from suppliers including Santa Cruz Biotechnology, Abcam, and Cell Signaling Technology.

Transduction markers may be selected from at least one of a truncated CD19 (tCD19; see Budde et al., Blood 122: 1660, 2013); a truncated human epidermal growth factor (tEGFR; see Wang et al., Blood 118: 1255, 2011); an extracellular domain of human CD34; and/or RQR8 which combines target epitopes from CD34 (see Fehse et al., Mol. Therapy 1(5 Pt 1); 448-456, 2000) and CD20 antigens (see Philip et al, Blood 124: 1277-1278).

In particular embodiments, a polynucleotide encoding an iCaspase9 construct (iCasp9) may be inserted into a CD33-targeting agent nucleotide construct as a suicide switch.

Control features may be present in multiple copies or can be expressed as distinct molecules with the use of a skipping element. For example, a CAR can have one, two, three, four or five tag cassettes and/or one, two, three, four, or five transduction markers could also be expressed. For example, embodiments can include a CD33-targeting agent having two Myc tag cassettes, or a His tag and an HA tag cassette, or a HA tag and a Softag 1 tag cassette, or a Myc tag and a SBP tag cassette. In particular embodiments, a transduction marker includes tEFGR. Exemplary transduction markers and cognate pairs are described in U.S. patent Ser. No. 13/463,247.

One advantage of including at least one control feature in cells genetically modified to express a CAR or TCR is that, if necessary or beneficial, the cells can be depleted following administration to a subject using the cognate binding molecule to a tag cassette.

In certain embodiments, CD33-targeting agents may be detected or tracked in vivo by using antibodies that bind with specificity to a control feature (e.g., anti-Tag antibodies), or by other cognate binding molecules that specifically bind the control feature, which binding partners for the control feature are conjugated to a fluorescent dye, radio-tracer, iron-oxide nanoparticle or other imaging agent known in the art for detection by X-ray, CT-scan, MRI-scan, PET-scan, ultrasound, flow-cytometry, near infrared imaging systems, or other imaging modalities (see, e.g., Yu et al., Theranostics 2:3, 2012).

Thus, CD33-targeting agents expressing at least one control feature can be more readily identified, isolated, sorted, tracked, and/or eliminated as compared to a CD33-targeting agent without a tag cassette.

In particular embodiments, the genetically-modified cells described herein are administered in combination with a treatment to target CD33-expressing cells using a CD33-targeting agent, such as an anti-CD33 antibody, an anti-CD33 immunotoxin (e.g., an antibody linked to a plant and/or bacterial toxin), an anti-CD33 antibody-drug conjugate (e.g., an antibody bound to a small molecule toxin), an anti-CD33 antibody-radioimmunoconjugate, an anti-CD33 bispecific antibody, an anti-CD33 bispecific antibody that binds CD33 and an immune activating epitope on an immune cell (e.g., a BiTE® (Amgen, Munich, Germany)), an anti-CD33 trispecific antibody, and/or an anti-CD33 CAR or TCR-modified T-cell.

### Applications

In various embodiments, a cell of a subject or system of the present disclosure is engineered to inactivate CD33. In various embodiments, CD33 is inactivated in one or more cells of a subject or system that has been contacted with, can be contacted with, or will be contacted with an anti-CD33 agent.

In various embodiments, CD33 is inactivated in one or more cells of a subject or system that has been identified as including cells of a CD33-expressing cancer where the subject has been contacted with, can be contacted with, or will be contacted with an anti-CD33 agent. In various embodiments, the cancer is a myeloid neoplasm. In various embodiments, the cancer is acute myeloid leukemia (AML), In various embodiments, the cancer is a myelodysplastic syndrome, acute biphenotypic leukemia, acute lymphocytic leukemia, chronic myelogenous leukemia, acute myeloid leukemia arising from previous myelodysplastic syndrome, acute promyelocytic leukemia, multiple myeloma, refractory anemia with excess blasts, secondary acute myeloid leukemia, system mastocytosis, skin cancer, therapy-related acute myeloid leukemia, or therapy-related myelodysplastic syndrome. In various embodiments, the cancer is lung cancer, colorectal cancer, head and neck cancer, stomach cancer, liver cancer, pancreatic cancer, urothelial cancer, prostate cancer, testis cancer, breast cancer, cervical cancer, endometrial cancer, ovarian cancer, melanoma, skin cancer, or lymphoma.

Particular embodiments include targeting any residual and/or non-therapeutic cells that express CD33 with the use of a CD33-targeting agent. In particular embodiments, genetically-modified therapeutic cells described herein can be administered alone or in combination with a CD33-targeting treatment, such as an anti-CD33 antibody, an anti-CD33 immunotoxin, an anti-CD33 antibody-drug conjugate, an anti-CD33 antibody-radioisotope conjugate, an anti-CD33 bispecific antibody, an anti-CD33 bispecific immune cell activating antibody, an anti-CD33 trispecific antibody, and/or an anti-CD33 chimeric antigen receptor (CAR) or T cell receptor (TCR) modified immune cell.

In various embodiments, agents for CD33 inactivation are administered together with (e.g., encoded by the same vector as) a therapeutic agent for treatment of a condition of CD33-expressing cells, such as HSCs. Accordingly, in various embodiments, agents for CD33 inactivation are administered to a subject having a condition that can be treated by engineering of HSCs. The present disclosure therefore further includes gene therapy reagents such as viral vectors that include nucleic acid sequences that encode a base editing system for CD33 inactivation together with a transgene encoding a further therapeutic payload expression product, e.g., for treatment of a condition that can be treated by HSC engineering.

Particular examples of therapeutic genes and/or gene products include γ-globin, Factor VIII, γC, JAK3, IL7RA, RAG1, RAG2, DCLRE1C, PRKDC, LIG4, NHEJ1, CD3D, CD3E, CD3Z, CD3G, PTPRC, ZAP70, LCK, AK2, ADA, PNP, WHN, CHD7, ORAI1, STIM1, CORO1A, CIITA, RFXANK, RFX5, RFXAP, RMRP, DKC1, TERT, TINF2, DCLRE1B, and SLC46A1; FANC family genes including FancA, FancB, FancC, FancD1 (BRCA2), FancD2, FancE, FancF, FancG, Fancl, FancJ (BRIP1), FancL, FancM, FancN (PALB2), FancO (RAD51C), FancP (SLX4), FancQ (ERCC4), FancR (RAD51), FancS (BRCA1), FancT (UBE2T), FancU (XRCC2), FancV (MAD2L2), and FancW (RFWD3); soluble CD40; CTLA; Fas L; antibodies to CD4, CD5, CD7, CD52, etc.; antibodies to IL1, IL2, IL6; an antibody to TCR specifically present on autoreactive T cells; IL4; IL10; IL12; IL13; ID Ra, sIL1RI, sIL1RII; sTNFRI; sTNFRII; antibodies to TNF; P53, PTPN22, and DRB1*1501/DQB1*0602; globin family genes; WAS; phox; dystrophin; pyruvate kinase; CLN3; ABCD1; arylsulfatase A; SFTPB; SFTPC; NLX2.1; ABCA3; GATA1; ribosomal protein genes; TERT; TERC; DKC1; TINF2; CFTR; LRRK2; PARK2; PARK7; PINK1; SNCA; PSEN1; PSEN2; APP; SOD1; TDP43; FUS; ubiquilin 2; C9ORF72 and other therapeutic genes described herein. In various embodiments of the present disclosure, a vector encodes a globin gene, wherein the globin protein encoded by the globin gene is selected from a γ-globin, a β-globin, and/or an α-globin. Globin genes of the present disclosure can include, e.g., one or more regulatory sequences such as a promoter operably linked to a nucleic acid sequence encoding a globin protein. As those of skill in the art will appreciate, each of γ-globin, β-globin, and/or α-globin is a component of fetal and/or adult hemoglobin and is therefore useful in various vectors disclosed herein. Various therapeutic genes and vector payloads are disclosed in related application No. PCT/US2020/040756, which is incorporated herein by reference in its entirety and with respect to therapeutic genes and payloads, e.g., for use in viral vectors.

As another example, a therapeutic gene can be selected to provide a therapeutically effective response against a lysosomal storage disorder. In particular embodiments, the lysosomal storage disorder is mucopolysaccharidosis (MPS), type I; MPS II or Hunter Syndrome; MPS III or Sanfilippo syndrome; MPS IV or Morquio syndrome; MPS V; MPS VI or Maroteaux-Lamy syndrome; MPS VII or sly syndrome; α-mannosidosis; β-mannosidosis; glycogen storage disease type I, also known as GSDI, von Gierke disease, or Tay Sachs; Pompe disease; Gaucher disease; Fabry disease. The therapeutic gene may be, for example a gene encoding or inducing production of an enzyme, or that otherwise causes the degradation of mucopolysaccharides in lysosomes. Exemplary therapeutic genes include IDUA or iduronidase, IDS, GNS, HGSNAT, SGSH, NAGLU, GUSB, GALNS, GLB1, ARSB, and HYAL1. Exemplary effective genetic therapies for lysosomal storage disorders may, for example, encode or induce the production of enzymes responsible for the degradation of various substances in lysosomes; reduce, eliminate, prevent, or delay the swelling in various organs, including the head (exp. macrocephaly), the liver, spleen, tongue, or vocal cords; reduce fluid in the brain; reduce heart valve abnormalities; prevent or dilate narrowing airways and prevent related upper respiratory conditions like infections and sleep apnea; reduce, eliminate, prevent, or delay the destruction of neurons, and/or the associated symptoms.

As another example, a therapeutic gene can be selected to provide a therapeutically effective response against a hyperproliferative disease. In particular embodiments, the hyperproliferative disease is cancer. The therapeutic gene may be, for example, a tumor suppressor gene, a gene that induces apoptosis, a gene encoding an enzyme, a gene encoding an antibody, or a gene encoding a hormone. Exemplary therapeutic genes and gene products include (in addition to those listed elsewhere herein) 101F6, 123F2 (RASSF1), 53BP2, abl, ABLI, ADP, aFGF, APC, ApoAI, ApoAIV, ApoE, ATM, BAI-1, BDNF, Beta*(BLU), bFGF, BLC1, BLC6, BRCA1, BRCA2, CBFA1, CBL, C-CAM, CNTF, COX-1, CSFIR, CTS-1, cytosine deaminase, DBCCR-1, DCC, Dp, DPC-4, E1A, E2F, EBRB2, erb, ERBA, ERBB, ETS1, ETS2, ETV6, Fab, FCC, FGF, FGR, FHIT, fms, FOX, FUS1, FYN, G-CSF, GDAIF, Gene 21 (NPRL2), Gene 26 (CACNA2D2), GM-CSF, GMF, gsp, HCR, HIC-1, HRAS, hst, IGF, IL-1, IL-2, IL-3, IL-5, IL-6, IL-7, IL-8, IL-9, IL-11, ING1, interferon α, interferon β, interferon γ, IRF-1, JUN, KRAS, LUCA-1 (HYAL1), LUCA-2 (HYAL2), LYN, MADH4, MADR2, MCC, mda7, MDM2, MEN-I, MEN-II, MLL, MMAC1, MYB, MYC, MYCL1, MYCN, neu, NF-1, NF-2, NGF, NOEY1, NOEY2, NRAS, NT3, NTS, OVCA1, p16, p21, p27, p57, p73, p300, PGS, PIM1, PL6, PML, PTEN, raf, Rap1A, ras, Rb, RB1, RET, rks-3, ScFv, scFV ras, SEM A3, SRC, TALI, TCL3, TFPI, thrombospondin, thymidine kinase, TNF, TP53, trk, T-VEC, VEGF, VHL, WT1, WT-1, YES, and zac1. Exemplary effective genetic therapies may suppress or eliminate tumors, result in a decreased number of cancer cells, reduced tumor size, slow or eliminate tumor growth, or alleviate symptoms caused by tumors.

As another example, a therapeutic gene can be selected to provide a therapeutically effective response against an infectious disease. In particular embodiments, the infectious disease is human immunodeficiency virus (HIV). The therapeutic gene may be, for example, a gene rendering immune cells resistant to HIV infection, or which enables immune cells to effectively neutralize the virus via immune reconstruction, polymorphisms of genes encoding proteins expressed by immune cells, genes advantageous for fighting infection that are not expressed in the patient, genes encoding an infectious agent, receptor or coreceptor; a gene encoding ligands for receptors or coreceptors; viral and cellular genes essential for viral replication including; a gene encoding ribozymes, antisense RNA, small interfering RNA (siRNA) or decoy RNA to block the actions of certain transcription factors; a gene encoding dominant negative viral proteins, intracellular antibodies, intrakines and suicide genes. Exemplary therapeutic genes and gene products include α2β1; αvβ3; αvβ5; αvβ63; BOB/GPR15; Bonzo/STRL-33/TYMSTR; CCR2; CCR3; CCRS; CCR8; CD4; CD46; CD55; CXCR4; aminopeptidase-N; HHV-7; ICAM; ICAM-1; PRR2/HveB; HveA; α-dystroglycan; LDLR/α2MR/LRP; PVR; PRR1/HveC; and laminin receptor. A therapeutically effective amount for the treatment of HIV, for example, may increase the immunity of a subject against HIV, ameliorate a symptom associated with AIDS or HIV, or induce an innate or adaptive immune response in a subject against HIV. An immune response against HIV may include antibody production and result in the prevention of AIDS and/or ameliorate a symptom of AIDS or HIV infection of the subject, or decrease or eliminate HIV infectivity and/or virulence.

The therapeutic administration of HSC can be used to treat a variety of adverse conditions including immune deficiency diseases, blood disorders, malignant cancers, infections, and radiation exposure (e.g., cancer treatment, accidental, or attack-based).

In various embodiments, methods and compositions of the present disclosure are used to treat, as a component of a therapy for, and/or in conjunction with a therapy for a rare hematology indication. Examples of rare hematology indications include, without limitation rare platelet disorders (e.g. Bernard-Soulier syndrome and Glanzmann thrombasthenia), Bone marrow failure conditions (e.g., Diamond-Blackfan anemia), other red cell disorders (e.g., pyruvate kinase deficiency), autoimmune rare hematologies (e.g., acquired thrombotic thrombocytopenic purpura (aTTP) and congenital thrombotic thrombocytopenic purpura (cTTP)), Primary Immunodeficiencies (PIDs) (e.g., Wiskott-Aldrich syndrome (WAS), Severe combined immunodeficiency due to adenosine deaminase deficiency (ADA-SCID), X-linked severe combined immunodeficiency (SCID-X1), DOCK 8 deficiency, major histocompatibility complex class II deficiency (MHC-II), and CD40/CD40L deficiencies), and other indications that include inborn errors of metabolism (IEMs) (e.g., hereditary hemochromatosis and phenylketonuria (PKU)).

As further particular examples of conditions that can be treated with HSC, more than 80 primary immune deficiency diseases are recognized by the World Health Organization. These diseases are characterized by an intrinsic defect in the immune system in which, in some cases, the body is unable to produce any or enough antibodies against infection. In other cases, cellular defenses to fight infection fail to work properly. Typically, primary immune deficiencies are inherited disorders.

One example of a primary immune deficiency is Fanconi anemia (FA). FA is an inherited blood disorder that leads to bone marrow (BM) failure. It is characterized, in part, by a deficient DNA-repair mechanism. At least 20% of patients with FA develop cancers such as acute myeloid leukemias and cancers of the skin, liver, gastrointestinal tract, and gynecological system. The skin and gastrointestinal tumors are usually squamous cell carcinomas. The average age of FA patients who develop cancer is 15 years for leukemia, 16 years for liver tumors, and 23 years for other tumors. The present disclosure includes the recognition that use of CD33 as a selection agent is particularly useful in the context of treatment of FA using a therapy that includes therapeutic HSCs at least in that various other means of therapeutic cell selection are not compatible with the biology of FA. The Fanconi anemia/BRCA (FA/BRCA) DNA damage repair pathway plays a pivotal role in the cellular response to DNA alkylating agents and greatly influences drug response in cancer treatment. Accordingly, FA patients are susceptible to adverse reaction to administration of alkylating agents such as BCNU, which is used as a selection agent for cells bearing the selectable marker MGMT^{P140K}. Accordingly, the present disclosure includes the recognition that use of CD33 inactivation as a selection agent in combination with an anti-CD33 agent has particular utility in HSC therapy for the treatment of FA.

X-linked severe combined immunodeficiency (SCID-X1) is both a cellular and humoral immune depletion caused by mutations in the common gamma chain gene (γC), which result in the absence of T and natural killer (NK) lymphocytes and the presence of nonfunctional B lymphocytes. SCID-X1 is fatal in the first two years of life unless the immune system is reconstituted, for example, through bone marrow transplant (BMT) or cell and gene therapy.

Secondary, or acquired, immune deficiencies are not the result of inherited genetic abnormalities, but rather occur in individuals in which the immune system is compromised by factors outside the immune system. Examples include trauma, viruses, chemotherapy, toxins, and pollution. Acquired immunodeficiency syndrome (AIDS) is an example of a secondary immune deficiency disorder caused by a virus, the human immunodeficiency virus (HIV), in which a depletion of T lymphocytes renders the body unable to fight infection.

FA, SCID, and other immune deficiencies or blood disorders as well as viral infections and cancer can be treated by a bone marrow transplant (BMT) or by administering hematopoietic cells that have been genetically modified to provide a functioning gene that the patient lacks. Therapeutic genes that can treat FA and SCID are described below. Therapeutic genes can also provide enzymes that are currently used for Enzyme replacement therapies (ERT) for lysosomal storage diseases such as Pompe disease (acid alpha-glucosidase), Gaucher disease (glucocerebrosidase), Fabry disease (alpha-galactosidase A), and Mucopolysaccharidosis type I (alpha-L-Iduronidase); blood-related cardiovascular diseases (e.g. familial apolipoprotein E deficiency and atherosclerosis (ApoE)); viral infections by expression of viral decoy receptors (e.g. for HIV-soluble CD4, or broadly neutralizing antibodies (bNAbs)) for HIV, chronic HCV, or HBV infections; and cancer (e.g. controlled expression of monoclonal antibodies (e.g. trastuzumab) or checkpoint inhibitors (e.g. aPDL1). Other additional uses are described in more detail elsewhere herein.

Immune deficiencies, blood cancers, and other blood-related disorders can be treated by a BMT or by administering hematopoietic cells. In some instances, the hematopoietic cells can be genetically modified to provide a functioning gene that the patient lacks.

In particular embodiments, methods of the present disclosure can be used to treat acquired thrombocytopenia, acute lymphoblastic leukemia (ALL), acute myelogenous leukemia (AML), adrenoleukodystrophy, agnogenic myeloid metaplasia, AIDS, amegakaryocytosic/congenital thrombocytopenia, aplastic anemia, ataxia telangiectasia, β-thalassemia major, Chediak-Higashi syndrome, chronic granulomatous disease, chronic lymphocytic leukemia (CLL), chronic myelogenous leukemia (CML), chronic myelomonocytic leukemia, common variable immune deficiency (CVID), complement disorders, congenital agammaglobulinemia, Diamond Blackfan syndrome, diffuse large B-cell lymphoma, Fabry disease (alpha-galactosidase A), familial erythrophagocytic lymphohistiocytosis, Fanconi's anemia, fetal maternal incompatibility, follicular lymphoma, Gaucher disease (glucocerebrosidase), hemolytic anemia, Hodgkin's lymphoma, Hurler's syndrome, hyper IgM, IgG subclass deficiency, hypogammaglobulinemia, immune thrombocytopenia purpura, juvenile myelomonocytic leukemia, leukemia, lymphoma, May-Hegglin syndrome, metachromatic leukodystrophy, mucopolysaccharidoses, mucopolysaccharidosis type I (alpha-L-Iduronidase), multiple myeloma (MM), myelodysplastic syndrome (MDS or myelodysplasia), myelofibrosis, non-Hodgkin's lymphoma (NHL), paroxysmal nocturnal hemoglobinuria (PNH), Pompe disease, primary immunodeficiency diseases with antibody deficiency, pure red cell aplasia, refractory anemia, SCID, selective IgA deficiency, severe aplastic anemia, Shwachman-Diamond-Blackfan anemia, sickle cell disease, specific antibody deficiency, systemic lupus erythematosus (SLE), thrombocytopenia, Wiskott-Aldridge syndrome, and X-linked agammaglobulinemia (XLA).

Additional exemplary cancers that may be treated include solid tumors, astrocytoma, atypical teratoid rhabdoid tumor, brain and central nervous system (CNS) cancer, breast cancer, carcinosarcoma, chondrosarcoma, chordoma, choroid plexus carcinoma, choroid plexus papilloma, clear cell sarcoma of soft tissue, gastrointestinal stromal tumor, glioblastoma, HBV-induced hepatocellular carcinoma, head and neck cancer, kidney cancer, lung cancer, malignant rhabdoid tumor, medulloblastoma, melanoma, meningioma, mesothelioma, neuroglial tumor, not otherwise specified (NOS) sarcoma, oligoastrocytoma, oligodendroglioma, osteosarcoma, ovarian cancer, ovarian clear cell adenocarcinoma, ovarian endometrioid adenocarcinoma, ovarian serous adenocarcinoma, pancreatic cancer, pancreatic ductal adenocarcinoma, pancreatic endocrine tumor, pineoblastoma, prostate cancer, renal cell carcinoma, renal medullo-carcinoma, rhabdomyosarcoma, sarcoma, schwannoma, skin squamous cell carcinoma, and stem cell cancer.

Particular examples of therapeutic genes and/or gene products to treat immune deficiencies can include genes associated with FA including: FancA, FancB, FancC, FancD1 (BRCA2), FancD2, FancE, FancF, FancG, Fancl, FancJ (BRIP1), FancL, FancM, FancN (PALB2), FancO (RAD51C), FancP (SLX4), FancQ (ERCC4), FancR (RAD51), FancS (BRCA1), FancT (UBE2T), FancU (XRCC2), FancV (MAD2L2), and FancW (RFWD3). Exemplary genes and proteins associated with FA include: Homo sapiens FANCA coding sequence; Homo sapiens FANCC coding sequence; Homo sapiens FANCE coding sequence; Homo sapiens FANCF coding sequence; Homo sapiens FANCG coding sequence; Homo sapiens FANCA AA; Homo sapiens FANCC AA; Homo sapiens FANCE AA; Homo sapiens FANCF AA; and Homo sapiens FANCG AA.

Particular examples of therapeutic genes and/or gene products to treat immune deficiencies can include genes associated with SCID including: γC, JAK3, IL7RA, RAG1, RAG2, DCLRE1C, PRKDC, LIG4, NHEJ1, CD3D, CD3E, CD3Z, CD3G, PTPRC, ZAP70, LCK, AK2, ADA, PNP, WHN, CHD7, ORAI1, STIM1, CORO1A, CIITA, RFXANK, RFX5, RFXAP, RMRP, DKC1, TERT, TINF2, DCLRE1 B, and SLC46A1. Exemplary genes and proteins associated with SCID include: exemplary codon optimized Human γC DNA; exemplary native Human γC DNA; exemplary native canine γC DNA; exemplary human γC AA; and exemplary native canine γC AA (91% conserved with human). Exemplary genes and proteins associated with SCID include: Homo sapiens JAK3 coding sequence; Homo sapiens PNP coding sequence; Homo sapiens ADA coding sequence; Homo sapiens RAG1 coding sequence; Homo sapiens RAG2 coding sequence; Homo sapiens JAK3 AA; Homo sapiens PNP AA; Homo sapiens ADA AA; Homo sapiens RAG1 AA; and Homo sapiens RAG2 AA.

Additional exemplary therapeutic genes can include or encode for clotting and/or coagulation factors such as factor VIII (FVIII), FVII, von Willebrand factor (VWF), FI, FII, FV, FX, FXI, and FXIII).

Additional examples of therapeutic genes and/or gene products include those that can provide a therapeutically effective response against diseases related to red blood cells and clotting. In particular embodiments, the disease is a hemoglobinopathy like thalassemia, or a SCD/trait. Exemplary therapeutic genes include F8 and F9.

Particular examples of therapeutic genes and/or gene products include γ-globin; soluble CD40; CTLA; Fas L; antibodies to CD4, CD5, CD7, CD52, etc.; antibodies to IL1, IL2, IL6; an antibody to TCR specifically present on autoreactive T cells; IL4; ID 0; ID 2; ID 3; ID Ra, sIL1RI, sIL1R11; sTNFRI; sTNFRII; antibodies to TNF; P53, PTPN22, and DRB1*1501/DQB1*0602; globin family genes; WAS; phox; dystrophin; pyruvate kinase (PK); CLN3; ABCD1; arylsulfatase A (ARSA); SFTPB; SFTPC; NLX2.1; ABCA3; GATA1; ribosomal protein genes; TERC; CFTR; LRRK2; PARK2; PARK7; PINK1; SNCA; PSEN1; PSEN2; APP; SOD1; TDP43; FUS; ubiquilin 2; C9ORF72 and other therapeutic genes described herein.

Particular embodiments include inserting or altering a gene selected from ABLI, AKT1, APC, ARSB, BCL11A, BLC1, BLC6, BRCA1, BRIP1, C46, CAS9, C-CAM, CBFAI, CBL, CCR5, CD19, CDA, C-MYC, CRE, CSCR4, CSFIR, CTS-I, CYB5R3, DCC, DHFR, DLL1, DMD, EGFR, ERBA, ERBB, EBRB2, ETSI, ETS2, ETV6, FCC, FGR, FOX, FUSI, FYN, GALNS, GLB1, GNS, GUSB, HBB, HBD, HBE1, HBG1, HBG2, HCR, HGSNAT, HOXB4, HRAS, HYAL1, ICAM-1, iCaspase, IDUA, IDS, JUN, KLF4, KRAS, LYN, MCC, MDM2, MGMT, MLL, MMACI, MYB, MEN-I, MEN-II, MYC, NAGLU, NANOG, NF-1, NF-2, NKX2.1, NOTCH, OCT4, p16, p2I, p27, p57, p73, PALB2, RAD51C, ras, at least one of RPL3 through RPL40, RPLPO, RPLP1, RPLP2, at least one of RPS2 through RPS30, RPSA, SGSH, SLX4, SOX2, VHL, and WT-I.

In addition to therapeutic genes and/or gene products, the transgene can also encode for therapeutic molecules, such as checkpoint inhibitor reagents, chimeric antigen receptor molecules specific to one or more cellular antigen (e.g. cancer antigen), and/or T-cell receptor specific to one or more cellular antigen (e.g. cancer antigen).

As indicated previously, FA is an inherited genetic disease characterized by fragile bone marrow cells and the inability to repair DNA damage, which accumulates in repopulating stem cells, resulting in eventual bone marrow failure. This disease can arise through mutations in any of a family of Fanconi-associated genes, with the most common of these mutations occurring in either the FANCA, FANCC, or FANCG genes. The current treatment protocol for patients is a bone marrow transplant from a matched donor, ideally from a sibling. However, the majority of patients will not have an appropriately matched sibling donor, and transplants from alternative donors are still associated with substantial toxicity and morbidity. For these patients, ongoing trials use an autologous transplant combined with new gene therapy approaches to introduce a corrected form of the mutated gene through collection and modification of the patient's own hematopoietic stem cells (e.g., Clinical Trial No. NCT01331018, accessible online at clinicaltrials.gov/ct2/show/NCT01331018).

In various embodiments, another problem for transplant recipients and recipients of other therapies disclosed herein is the conditioning regimens used in some embodiments to prepare the marrow compartment for infused cells to engraft. In various treatment scenarios, it is important to remove or eliminate cells of a patient's existing hematopoietic system or to reduce the number and/or concentration of one or more cells thereof (e.g., HSCs and/or HSPCs) to avoid leaving, and/or reduce the number or concentration of remaining, diseased cells following the treatment. This is in part because, if left, diseased residual cells can lead to malignancies later in life. Removing a patient's existing hematopoietic system is most often accomplished utilizing a process referred to as conditioning. In various embodiments, this type of conditioning precedes administration of therapeutic HSCs (e.g., engineered HSCs). Traditionally, conditioning has involved the delivery of maximally tolerated doses of chemotherapeutic agents with nonoverlapping toxicities, with or without radiation. Current conditioning regimens involve total body irradiation (TBI) and/or cytotoxic drugs. These regimens are non-targeted, genotoxic, and have multiple short- and long-term adverse effects such as an increased risk of developing DNA repair disorders, interstitial pneumonitis, idiopathic pulmonary fibrosis, reduced lung pulmonary function, renal damage, sinusoidal obstruction syndrome (SOS), infertility, cataract formation, hyperthyroidism, thyroiditis, and secondary cancers. Besides morbidity, these regimens are also associated with significant mortality. Therefore, methods to reduce or eliminate the need for conditioning in these patients is desperately needed.

In view of various challenges associated with conditioning, regimens that minimize and/or reduce the intensity of conditioning have been developed but in various embodiments remain problematic. Since chemotherapy or other DNA damaging agents are not well tolerated in these patients due to their underlying disease condition, in autologous transplants, gene modified cells are re-administered without prior conditioning. While safer, avoidance of conditioning potentially prevents efficient engraftment of corrected cells into the marrow niche where they can begin contributing to hematopoietic development. In the allogeneic transplant setting initial conditioning regimens included TBI and cyclophosphamide (Cy), however, significant mortality was observed secondary to graft-versus-host disease (GVHD) and Cy toxicity including hemorrhagic cystitis, mucositis, and cardiac failure. For this reason, reduced-intensity conditioning (RIC) is now used for FA patients which includes low-dose Cy, fludarabine, and anti-thymocyte globulin. Although overall survival has improved using RIC, late complications continue to be an issue whether associated with conditioning, GVHD, or from disease-related complications.

For the reasons noted above, FA is an ideal candidate for autologous gene therapy, wherein the patient's own HSC can supply a functional FA gene, thereby diminishing GVHD risk. Importantly, the rationale for autologous genetic correction, even in a small number of cells, is supported by the spontaneous correction of the mutated FA gene documented in a few FA patients and resulting improvement in hematologic parameters. This “somatic mosaicism” occurs in single cell clones that can then sustain hematopoiesis over years without the requirement for marrow conditioning. A number of preclinical studies have demonstrated in vitro gene delivery by viral vectors, resulting in FA phenotype correction as demonstrated by protection from DNA crosslinking agents, such as mitomycin C (MMC). Integrating retroviral vectors encoding FANCA or FANCC cDNA were used to transduce FA murine hematopoietic progenitor cells, restore resistance of colony forming cells to MMC, and repopulate murine homozygous deficient models. As a result, several clinical trials have been conducted. All of these trials have attempted collection of FA patient HSPC by selecting CD34⁺ cells for ex vivo gene transfer and subsequent reinfusion to limit off-target transduction for reasons of both safety and efficacy. One important remaining obstacle with autologous gene therapy is the presence of residual FA hematopoiesis that can result in myeloid malignancy, a scenario that could be minimized with the inclusion of the disclosed strategy to eliminate non-corrected or host FA cells. The current disclosure and treatment methods address this concern.

In particular embodiments, therapeutic efficacy can be observed through mouse models of FA transplantation that have been used to study ex vivo gene therapy of HSPCs. One such model includes a functional knockout of the FANCA gene, resulting in fragile marrow of these mice that are thus unable to form healthy colonies when bone marrow is plated in outgrowth assays in the presence of even low levels of MMC, a DNA damaging agent. Healthy heterozygote littermates exhibit bone marrow colony forming potential regardless of MMC presence, whereas FANCA mice are demonstrated to have a significant decrease in colony forming potential with increasing MMC concentration. This mimics the clinical setting where patient stem cells exhibit a similar phenotype when exposed to DNA damaging agents. The use of low-dose Cy for bone marrow transplant in this mouse model has been demonstrated. Without some form of pre-conditioning prior to transplant, donor cells can home to the bone marrow; however, they do not contribute to peripheral hematopoiesis. This underscores the need to both clear FANCA-deficient stem cell populations and promote engraftment and hematopoietic development of transplanted donor or gene-corrected autologous cell populations.

In particular embodiments, therapeutic efficacy for FA (and other immune deficiency disorders) can be observed through lymphocyte reconstitution, improved clonal diversity and thymopoiesis, reduced infections, and/or improved patient outcome. Therapeutic efficacy can also be observed through one or more of weight gain and growth, improved gastrointestinal function (e.g., reduced diarrhea), reduced upper respiratory symptoms, reduced fungal infections of the mouth (thrush), reduced incidences and severity of pneumonia, reduced meningitis and blood stream infections, and reduced ear infections. In particular embodiments, treating FA with methods of the present disclosure include increasing resistance of BM derived cells to mitomycin C (MMC). In particular embodiments, the resistance of BM derived cells to MMC can be measured by a cell survival assay in methylcellulose and MMC.

In particular embodiments, methods of the present disclosure can be used to treat SCID-X1. In particular embodiments, methods of the present disclosure can be used to treat SCID (e.g., JAK 3 kinase deficiency SCID, purine nucleoside phosphorylase (PNP) deficiency SCID, adenosine deaminase (ADA) deficiency SCID, MHC class II deficiency or recombinase activating gene (RAG) deficiency SCID). In particular embodiments, therapeutic efficacy can be observed through lymphocyte reconstitution, improved clonal diversity and thymopoiesis, reduced infections, and/or improved patient outcome. Therapeutic efficacy can also be observed through one or more of weight gain and growth, improved gastrointestinal function (e.g., reduced diarrhea), reduced upper respiratory symptoms, reduced fungal infections of the mouth (thrush), reduced incidences and severity of pneumonia, reduced meningitis and blood stream infections, and reduced ear infections. In particular embodiments, treating SCID-X1 with methods of the present disclosure include restoring functionality to the γC-dependent signaling pathway. The functionality of the γC-dependent signaling pathway can be assayed by measuring tyrosine phosphorylation of effector molecules STAT3 and/or STAT5 following in vitro stimulation with IL-21 and/or IL-2, respectively. Tyrosine phosphorylation of STAT3 and/or STAT5 can be measured by intracellular antibody staining.

Particular embodiments include treatment of secondary, or acquired, immune deficiencies such as immune deficiencies caused by trauma, viruses, chemotherapy, toxins, and pollution. As previously indicated, acquired immunodeficiency syndrome (AIDS) is an example of a secondary immune deficiency disorder caused by a virus, the human immunodeficiency virus (HIV), in which a depletion of T lymphocytes renders the body unable to fight infection. Thus, as another example, a gene can be selected to provide a therapeutically effective response against an infectious disease. In particular embodiments, the infectious disease is human immunodeficiency virus (HIV). The therapeutic gene may be, for example, a gene rendering immune cells resistant to HIV infection, or which enables immune cells to effectively neutralize the virus via immune reconstruction, polymorphisms of genes encoding proteins expressed by immune cells, genes advantageous for fighting infection that are not expressed in the patient, genes encoding an infectious agent, receptor or coreceptor; a gene encoding ligands for receptors or coreceptors; viral and cellular genes essential for viral replication including; a gene encoding ribozymes, antisense RNA, small interfering RNA (siRNA) or decoy RNA to block the actions of certain transcription factors; a gene encoding dominant negative viral proteins, intracellular antibodies, intrakines and suicide genes. Exemplary therapeutic genes and gene products include α2β1; αvβ3; αvβ5; αvβ63; BOB/GPR15; Bonzo/STRL-33/TYMSTR; CCR2; CCR3; CCR5; CCR8; CD4; CD46; CD55; CXCR4; aminopeptidase-N; HHV-7; ICAM; ICAM-1; PRR2/HveB; HveA; α-dystroglycan; LDLR/α2MR/LRP; PVR; PRR1/HveC; and laminin receptor. A therapeutically effective amount for the treatment of HIV, for example, may increase the immunity of a subject against HIV, ameliorate a symptom associated with AIDS or HIV, or induce an innate or adaptive immune response in a subject against HIV. An immune response against HIV may include antibody production and result in the prevention of AIDS and/or ameliorate a symptom of AIDS or HIV infection of the subject or decrease or eliminate HIV infectivity and/or virulence.

In particular embodiments, methods of the present disclosure can be used to treat hypogammaglobulinemia. Hypogammaglobulinemia is caused by a lack of B-lymphocytes and is characterized by low levels of antibodies in the blood. Hypogammaglobulinemia can occur in patients with chronic lymphocytic leukemia (CLL), multiple myeloma (MM), non-Hodgkin's lymphoma (NHL) and other relevant malignancies as a result of both leukemia-related immune dysfunction and therapy-related immunosuppression. Patients with acquired hypogammaglobulinemia secondary to such hematological malignancies, and those patients receiving post-HSPC transplantation are susceptible to bacterial infections. The deficiency in humoral immunity is largely responsible for the increased risk of infection-related morbidity and mortality in these patients, especially by encapsulated microorganisms. For example, Streptococcus pneumoniae, Hemophilus influenzae, and Staphylococcus aureus, as well as Legionella and Nocardia spp. are frequent bacterial pathogens that cause pneumonia in patients with CLL. Opportunistic infections such as Pneumocystis carinii, fungi, viruses, and mycobacteria also have been observed. The number and severity of infections in these patients can be significantly reduced by administration of immune globulin (Griffiths et al., Blood 73: 366-368, 1989; Chapel et al., Lancet 343: 1059-1063, 1994).

In particular embodiments, a therapeutically effective treatment induces or increases expression of fetal hemoglobin (HbF), induces or increases production of hemoglobin and/or induces or increases production of β-globin. In particular embodiments, a therapeutically effective treatment improves blood cell function, and/or increases oxygenation of cells. Treatments that induce and/or increase expression of HbF as disclosed herein can be useful in the treatment of various conditions disclosed herein, including without limitation thalassemia (e.g., β-thalassemia) and sickle cell disease.

In the context of cancers, therapeutically effective amounts have an anti-cancer effect. An anti-cancer effect can be quantified by observing a decrease in the number of cancer cells, a decrease in the number of metastases, a decrease in cancer volume, an increase in life expectancy, induction of apoptosis of cancer cells, induction of cancer cell death, inhibition of cancer cell proliferation, inhibition of tumor (e.g., solid tumor) growth, prevention of metastasis, prolongation of a subject's life, and/or reduction of relapse or re-occurrence of the cancer following treatment.

In particular embodiments, methods of the present disclosure can restore BM function in a subject in need thereof. In particular embodiments, restoring BM function can include improving BM repopulation with gene corrected cells as compared to a subject in need thereof that is not administered a therapy described herein. Improving BM repopulation with gene corrected cells can include increasing the percentage of cells that are gene corrected. In particular embodiments, the cells are selected from white blood cells and BM derived cells. In particular embodiments, the percentage of cells that are gene corrected can be measured using an assay selected from quantitative real time PCR and flow cytometry.

In particular embodiments, methods of the present disclosure can restore T-cell mediated immune responses in a subject in need thereof. Restoration of T-cell mediated immune responses can include restoring thymic output and/or restoring normal T lymphocyte development.

In particular embodiments, methods of the present disclosure can improve the kinetics and/or clonal diversity of lymphocyte reconstitution in a subject in need thereof. In particular embodiments, improving the kinetics of lymphocyte reconstitution can include increasing the number of circulating T lymphocytes to within a range of a reference level derived from a control population. In particular embodiments, improving the kinetics of lymphocyte reconstitution can include increasing the absolute CD3+ lymphocyte count to within a range of a reference level derived from a control population. A range can be a range of values observed in or exhibited by normal (i.e., non-immuno-compromised) subjects for a given parameter. In particular embodiments, improving the kinetics of lymphocyte reconstitution can include reducing the time required to reach normal lymphocyte counts as compared to a subject in need thereof not administered a therapy described herein. In particular embodiments, improving the kinetics of lymphocyte reconstitution can include increasing the frequency of gene corrected lymphocytes as compared to a subject in need thereof not administered a therapy described herein. In particular embodiments, improving the kinetics of lymphocyte reconstitution can include increasing diversity of clonal repertoire of gene corrected lymphocytes in the subject as compared to a subject in need thereof not administered a gene therapy described herein. Increasing diversity of clonal repertoire of gene corrected lymphocytes can include increasing the number of unique retroviral integration site (RIS) clones as measured by a RIS analysis.

In particular embodiments, restoring thymic output can include restoring the frequency of CD3+ T cells expressing CD45RA in peripheral blood to a level comparable to that of a reference level derived from a control population. In particular embodiments, restoring thymic output can include restoring the number of T cell receptor excision circles (TRECs) per 10⁶ maturing T cells to a level comparable to that of a reference level derived from a control population. The number of TRECs per 10⁶ maturing T cells can be determined as described in Kennedy et al., Vet Immunol Immunopathol 142: 36-48, 2011.

In particular embodiments, restoring normal T lymphocyte development includes restoring the ratio of CD4+ cells: CD8+ cells to 2. In particular embodiments, restoring normal T lymphocyte development includes detecting the presence of αβ TCR in circulating T-lymphocytes. The presence of αβ TCR in circulating T-lymphocytes can be detected, for example, by flow cytometry using antibodies that bind an α and/or β chain of a TCR. In particular embodiments, restoring normal T lymphocyte development includes detecting the presence of a diverse TCR repertoire comparable to that of a reference level derived from a control population. TCR diversity can be assessed by TCRVβ spectratyping, which analyzes genetic rearrangement of the variable region of the TCRβ gene. Robust, normal spectratype profiles can be characterized by a Gaussian distribution of fragments sized across 17 families of TCRVβ segments. In particular embodiments, restoring normal T lymphocyte development includes restoring T-cell specific signaling pathways. Restoration of T-cell specific signaling pathways can be assessed by lymphocyte proliferation following exposure to the T cell mitogen phytohemagglutinin (PHA). In particular embodiments, restoring normal T lymphocyte development includes restoring white blood cell count, neutrophil cell count, monocyte cell count, lymphocyte cell count, and/or platelet cell count to a level comparable to a reference level derived from a control population.

In particular embodiments, methods of the present disclosure can normalize primary and secondary antibody responses to immunization in a subject in need thereof. Normalizing primary and secondary antibody responses to immunization can include restoring B-cell and/or T-cell cytokine signaling programs functioning in class switching and memory response to an antigen. Normalizing primary and secondary antibody responses to immunization can be measured by a bacteriophage immunization assay. In particular embodiments, restoration of B-cell and/or T-cell cytokine signaling programs can be assayed after immunization with the T-cell dependent neoantigen bacteriophage ΨX174. In particular embodiments, normalizing primary and secondary antibody responses to immunization can include increasing the level of IgA, IgM, and/or IgG in a subject in need thereof to a level comparable to a reference level derived from a control population. In particular embodiments, normalizing primary and secondary antibody responses to immunization can include increasing the level of IgA, IgM, and/or IgG in a subject in need thereof to a level greater than that of a subject in need thereof not administered a gene therapy described herein. The level of IgA, IgM, and/or IgG can be measured by, for example, an immunoglobulin test. In particular embodiments, the immunoglobulin test includes antibodies binding IgG, IgA, IgM, kappa light chain, lambda light chain, and/or heavy chain. In particular embodiments, the immunoglobulin test includes serum protein electrophoresis, immunoelectrophoresis, radial immunodiffusion, nephelometry and turbidimetry. Commercially available immunoglobulin test kits include MININEPH™ (Binding site, Birmingham, UK), and immunoglobulin test systems from Dako (Glostrup, Denmark) and Dade Behring (Marburg, Germany). In particular embodiments, a sample that can be used to measure immunoglobulin levels includes a blood sample, a plasma sample, a cerebrospinal fluid sample, and a urine sample.

In particular embodiments, therapeutically effective amounts may provide function to immune and other blood cells, reduce or eliminate an immune-mediated condition; and/or reduce or eliminate a symptom of the immune-mediated condition.

In particular embodiments, particular methods of use include the treatment of conditions wherein corrected cells have a selective advantage over non-corrected cells. For example, in FA and SCID, corrected cells have an advantage and only transducing the therapeutic gene into a “few” HSPCs is sufficient for therapeutic efficacy.

The actual dose and amount of a therapeutic formulation and/or composition administered to a particular subject can be determined by a physician, veterinarian, or researcher taking into account parameters such as physical and physiological factors including target; body weight; type of condition; severity of condition; upcoming relevant events, when known; previous or concurrent therapeutic interventions; idiopathy of the subject; and route of administration, for example. In addition, in vitro and in vivo assays can optionally be employed to help identify optimal dosage ranges.

Therapeutically effective amounts of cell-based compositions can include 10⁴ to 10⁹ cells/kg body weight, or 10³ to 10¹¹ cells/kg body weight. Exemplary doses may include greater than 10² cells, greater than 10³ cells, greater than 10⁴ cells, greater than 10⁵ cells, greater than 10⁶ cells, greater than 10⁷ cells, greater than 10⁸ cells, greater than 10⁹ cells, greater than 10¹⁰ cells, or greater than 10¹¹ cells.

Therapeutically effective amounts of protein-based compounds within CD33 targeting compositions can include 0.1 to 5 μg or μg/kg, or from 0.5 to 1 μg/kg. In other examples, a dose can include 1 μg or μg/kg, 15 μg or μg/kg, 30 μg or μg/kg, 50 μg or μg/kg, 55 μg or μg/kg, 70 μg or μg/kg, 90 μg or μg/kg, 150 μg or μg/kg, 350 μg or μg/kg, 500 μg or μg/kg, 750 μg or μg/kg, 1000 μg or μg/kg, 0.1 to 5 mg/kg or from 0.5 to 1 mg/kg. In other examples, a dose can include 1 mg/kg, 10 mg/kg, 30 mg/kg, 50 mg/kg, 70 mg/kg, 100 mg/kg, 300 mg/kg, 500 mg/kg, 700 mg/kg, 1000 mg/kg or more.

Therapeutically effective amounts can be administered through any appropriate administration route such as by, injection, infusion, perfusion, and more particularly by administration by one or more of bone marrow, intravenous, intradermal, intraarterial, intranodal, intralymphatic, intraperitoneal injection, infusion, or perfusion). Administration of CD33-targeting agents can additionally be through oral administration, inhalation, or implantation.

Therapeutically effective amounts can be achieved by administering single or multiple doses during the course of a treatment regimen, depending on, for example, the particular treatment protocol being implemented. In particular embodiments, the treatment protocol may be dictated by a clinical trial protocol or an FDA-approved treatment protocol.

As disclosed herein, applications of the present disclosure that include, e.g., base editing compositions for inactivation of CD33 and uses thereof, provide various benefits, e.g., as compared to reference CRISPR editing systems (e.g., a CRISPR editing system with a same or similar editing target site). In various embodiments, base editing systems and uses thereof disclosed herein do not cause and/or are less prone to double-stranded DNA breaks, and/or cause fewer or at a lower rate, and can entail a decreased risk of translocation and/or intra-chromosomal rearrangement compared to a reference CRISPR editing system. In various embodiments, base editing systems and uses thereof disclosed herein do not cause and/or are less prone to causing deletion of one or more nucleotide positions at target base editing sites compared to a reference CRISPR editing system. In various embodiments, base editing systems and uses thereof disclosed herein do not cause, and/or are less prone to causing, and/or causes a reduced DNA emergency repair response (e.g., reduced emergency response agent activity and/or expression as compared to a reference CRISPR editing system). Reduced DNA damage caused by base editing systems as compared to reference CRISPR editing systems accordingly reduces risk of future malignancy, e.g., malignancy resulting directly or indirectly from DNA damage caused by, or off-target effects of, an editing system.

Moreover, base editing systems of the present disclosure are able to edit multiple target sites in a single cell using multiple gRNAs that are simultaneously present and/or expressed in the single cell. This contrasts with CRISPR systems that typically cause high levels of genotoxicity under conditions in which multiple gRNAs are simultaneously present and/or expressed for CRISPR editing of multiple target sites in a single cell. Accordingly, the present disclosure includes the recognition that methods and compositions of the present disclosure that include base editing systems for inactivation of CD33 can be multiplexed with use of gRNAs corresponding to additional editing targets in single cells (e.g., additional editing targets that contribute to treatment of a condition or disease) can be used with significantly lower genotoxicity and/or cytotoxicity as compared to use of reference multiplexed CRISPR editing systems.

Further advantages of base editing compositions and methods of the present disclosure reduce or eliminate the need for genotoxic preconditioning prior to or in association with therapy, e.g., to ablate, reduce, and/or eliminate HSC and/or HSPC cells and populations, e.g., prior to a treatment such as an HSC transplant, HSPC transplant, bone marrow transplant, administration of ex vivo engineered HSCs and/or HSPCs, and/or in vivo engineering of HSCs and/or HSPCs.

Those of skill in the art will appreciate from the present disclosure that base edited HSCs (e.g., CD33 base edited cells) in various embodiments demonstrate superior survival and/or proliferation in vivo as compared to reference HSCs edited by CRISPR editing systems. Moreover, the present disclosure includes recognition of the surprising differential in in vivo survival, proliferation, and/or engraftment of HSCs edited by a base editing system as compared to reference HSCs edited by CRISPR editing system. Methods of measuring HSC survival, proliferation, and/or engraftment are known in the art and disclosed herein. Those of skill in the art will appreciate from the present disclosure that survival and/or proliferation (and thereby genotoxicity and/or cytotoxicity) can be measured, e.g., as percentage of engineered HSCs in a subject to which engineered HSCs were administered, e.g., as measured by flow cytometry or sequencing approaches, or by in vivo, in vivo, or ex vivo measurement of cell survival over time, e.g., in culture, e.g., in culture comprising an admixture of engineered and non-engineered cells.

The Exemplary Embodiments and Examples below are included to demonstrate particular embodiments of the disclosure. Those of ordinary skill in the art will recognize in light of the present disclosure that many changes can be made to the specific embodiments disclosed herein and still obtain a like or similar result without departing from the spirit and scope of the disclosure.

### First Set of Exemplary Embodiments

1. A cell genetically modified with a base-editing system wherein the genetic modification reduces CD33 expression.
2. A cell of embodiment 1, wherein the base-editing system includes a cytosine-base editing system that replaces guanine/cytosine base pair with an adenine/thymine base pair.
3. A cell of embodiment 2, wherein the cytosine-base editing system includes cytosine deaminase.
4. A cell of embodiment 1, wherein the base-editing system includes a CRISPR-based nuclease, a zinc finger nuclease or a transcription activator like effector nuclease.
5. A cell of embodiment 4, wherein the nuclease has nickase function.
6. A cell of embodiment 4, wherein the CRISPR-based nuclease includes Cas9.
7. A cell of embodiment 1, wherein the base-editing system includes a DNA glycosylate inhibitor.
8. A cell of embodiment 7, wherein the DNA glycosylate inhibitor includes a uracil DNA glycosylate inhibitor.
9. A cell of embodiment 1, wherein the base-editing system is selected from BEI, BE2, BE3, HF-BE3, BE4, BE4max, BE4-GAM, YE1-BE3, EE-BE3, YE2-BE3, YEE-BE3, VQR-BE3, VRER-BE3, Sa-BE3, SA-BE4, SaBE4-Gam, SaKKH-BE3, Cas12a-BE, Target-AID, Target-AID-NG, xBE3, eA3A-BE3, A3A-BE3, and BE-PLUS.
10. A cell of embodiment 1, wherein the base-editing system is BE4max and/or SaBE4-Gam.
11. A cell of embodiment 1, wherein the genetic modification inactivates the intron1 splicing donor site of CD33.
12. A cell of embodiment 1, wherein the genetic modification results in introduction of a stop codon within the CD33-coding sequence.
13. A cell of embodiment 1, wherein the genetic modification results in introduction of a stop codon within exon 2 of the CD33-coding sequence.
14. A cell of embodiment 1, wherein the cell is a hematopoietic stem and progenitor cell (HSPC).
15. A cell of embodiment 1, wherein the cell is a CD34+CD45RA-CD90+ HSC.
16. A cell of embodiment 1, further genetically modified to include a therapeutic gene.
17. A cell of embodiment 16, wherein the therapeutic gene includes FancA, FancB, FancC, FancD1, FancD2, FancE, FancF, FancG, Fancl, FancJ, FancL, FancM, FancN, FancO, FancP, FancQ, FancR, FancS, FancT, FancU, FancV, or FancW or encodes a checkpoint inhibitor, a gene editing molecule, a chimeric antigen receptor that specifically binds a cellular antigen (e.g. a cancer antigen or a viral antigen), and/or a T-cell receptor that specifically binds a cellular antigen (e.g. a cancer antigen or a viral antigen).
18. A cell of embodiment 16, wherein the therapeutic gene includes □C, JAK3, IL7RA, RAG1, RAG2, DCLRE1C, PRKDC, LIG4, NHEJ1, CD3D, CD3E, CD3Z, CD3G, PTPRC, ZAP70, LCK, AK2, ADA, PNP, WHN, CHD7, ORAI1, STIM1, CORO1A, CIITA, RFXANK, RFX5, RFXAP, RMRP, DKC1, TERT, TINF2, DCLRE1B, or SLC46A1.
19. A cell of embodiment 16, wherein the therapeutic gene includes factor VIII (FVIII), FVII, von Willebrand factor (VWF), FI, FII, FV, FX, FXI, or FXIII).
20. A cell of embodiment 16, wherein the therapeutic gene includes F8 or F9.
21. A cell of embodiment 16, wherein the therapeutic gene includes γ-globin; soluble CD40; CTLA; Fas L; antibodies to CD4, CD5, CD7, CD52, etc.; antibodies to IL1, IL2, IL6; an antibody to TCR specifically present on autoreactive T cells; IL4; IL10; IL12; IL13; ID Ra, sIL1RI, sIL1RII; sTNFRI; sTNFRII; antibodies to TNF; P53, PTPN22, and DRB1*1501/DQB1*0602; globin family genes; WAS; phox; dystrophin; pyruvate kinase (PK); CLN3; ABCD1; arylsulfatase A (ARSA); SFTPB; SFTPC; NLX2.1; ABCA3; GATA1; ribosomal protein genes; TERC; CFTR; LRRK2; PARK2; PARK7; PINK1; SNCA; PSEN1; PSEN2; APP; SOD1; TDP43; FUS; ubiquilin 2; or C9ORF72.
22. A cell of embodiment 16, wherein the therapeutic gene includes ABLI, AKT1, APC, ARSB, BCL11A, BLC1, BLC6, BRCA1, BRCA2, BRIP1, C46, CAS9, C-CAM, CBFAI, CBL, CCR5, CD19, CDA, C-MYC, CRE, CSCR4, CSFIR, CTS-I, CYB5R3, DCC, DHFR, DLL1, DMD, EGFR, ERBA, ERBB, EBRB2, ETSI, ETS2, ETV6, FCC, FGR, FOX, FUSI, FYN, GALNS, GLB1, GNS, GUSB, HBB, HBD, HBE1, HBG1, HBG2, HCR, HGSNAT, HOXB4, HRAS, HYAL1, ICAM-1, iCaspase, IDUA, IDS, JUN, KLF4, KRAS, LYN, MCC, MDM2, MGMT, MLL, MMACI, MYB, MEN-I, MEN-II, MYC, NAGLU, NANOG, NF-1, NF-2, NKX2.1, NOTCH, OCT4, p16, p2I, p27, p57, p73, PALB2, RAD51C, ras, at least one of RPL3 through RPL40, RPLPO, RPLP1, RPLP2, at least one of RPS2 through RPS30, RPSA, SGSH, SLX4, SOX2, VHL, or WT-I.
23. A pharmaceutical formulation including a cell or population of cells of embodiment 1 and a pharmaceutically acceptable carrier.
24. A kit including a cell of embodiment 1 and a CD33-targeting agent.
25. A kit of embodiment 24, wherein the CD33-targeting agent includes an anti-CD33 antibody, an anti-CD33 immunotoxin, an anti-CD33 antibody-drug conjugate, an anti-CD33 antibody-radioisotope conjugate, an anti-CD33 bispecific antibody, an anti-CD33 bispecific immune cell engaging antibody, an anti-CD33 trispecific antibody, and/or an anti-CD33 chimeric antigen receptor (CAR) with one or more binding domains.
26. A kit of embodiment 24, wherein the CD33-targeting agent includes hp67.6, lintuzumab, SGN-CD33A, and/or AMG 330.
27. A kit of embodiment 24, wherein the CD33-targeting agent includes a binding domain derived from hp67.6, lintuzumab, SGN-CD33A, and/or AMG 330.
28. A kit of embodiment 24, wherein the CD33-targeting agent includes the CDRs of hp67.6, lintuzumab, SGN-CD33A, and/or AMG 330.
29. A kit of embodiment 24, wherein the CD33-targeting agent includes an antibody-drug conjugate or an antibody-radioisotope conjugate wherein the drug or radioisotope are selected from taxol, taxane, cytochalasin B, gramicidin D, ethidium bromide, emetine, mitomycin, etoposide, tenoposide, vincristine, vinblastine, colchicin, doxorubicin, daunorubicin, dihydroxy anthracinedione, mitoxantrone, mithramycin, maytansinoid, dolastatin, auristatin, calicheamicin, pyrrolobenzodiazepine, nemorubicin PNU-159682, anthracycline, vinca alkaloid, trichothecene, CC1065, camptothecin, elinafide, actinomycin D, 1-dehydrotestosterone, glucocorticoids, procaine, tetracaine, lidocaine, propranolol, puromycin, ricin, CC-1065, duocarmycin, diphtheria toxin, snake venom, cobra venom, mistletoe lectin, modeccin, pokeweed antiviral protein, saporin, Bryodin 1, bouganin, gelonin, Pseudomonas exotoxin, iodine-131, indium-111, yttrium-90, lutetium-177, astatine-211, bismuth-212, and/or bismuth-213 and/or wherein the antibody-drug conjugate includes GO.
30. A kit of embodiment 24, wherein the CD33-targeting agent includes a linker.
31. A kit of embodiment 24, wherein the CD33-targeting agent includes a bispecific antibody including a combination of binding variable chains or a binding CDR combination of hp67.6, lintuzumab, SGN-CD33A, and/or AMG 330.
32. A kit of embodiment 24, wherein the CD33-targeting agent includes a bispecific antibody including at least one binding domain that activates an immune cell.
33. A kit of embodiment 32, wherein the immune cell is a T-cell, natural killer (NK) cell, or a macrophage.
34. A kit of embodiment 32, wherein the binding domain that activates an immune cell binds CD3, CD28, CD8, NKG2D, CD8, CD16, KIR2DL4, KIR2DS1, KIR2DS2, KIR3DS1, NKG2C, NKG2E, NKG2D, NKp30, NKp44, NKp46, NKp80, DNAM-1, CD11b, CD11c, CD64, CD68, CD119, CD163, CD206, CD209, F4/80, IFGR2, Toll-like receptors 1-9, IL-4Ra, or MARCO.
35. A kit of embodiment 32, wherein the binding domains of the bispecific antibody are joined through a linker.
36. A kit of embodiment 24, wherein the CD33-targeting agent includes a chimeric antigen receptor (CAR) including a binding domain that specifically binds CD33.
37. A kit of embodiment 36, wherein the effector domain of the CAR is selected from 4-1BB, CD3E, CD3δ, CD3ζ, CD27, CD28, CD79A, CD79B, CARD11, DAP10, FcRα, FcRβ, FcRγ, Fyn, HVEM, ICOS, Lck, LAG3, LAT, LRP, NOTCH1, Wnt, NKG2D, OX40, ROR2, Ryk, SLAMF1, Slp76, pTα, TCRα, TCRβ, TRIM, Zap70, PTCH2, or any combination thereof.
38. A kit of embodiment 36, wherein the CAR includes a cytoplasmic signaling sequence derived from CD3 zeta, FcR gamma, CD3 gamma, CD3 delta, CD3 epsilon, CDS, CD22, CD79a, CD79b, or CD66d.
39. A kit of embodiment 36, wherein the CAR includes an intracellular signaling domain and a costimulatory signaling region.
40. A kit of embodiment 36, wherein the costimulatory signaling region includes the intracellular domain of CD27, CD28, 4-1 BB, OX40, CD30, CD40, lymphocyte function-associated antigen-1, CD2, CD7, LIGHT, NKG2C, or B7-H3.
41. A kit of embodiment 36, wherein the CAR includes a spacer region.
42. A kit of embodiment 36, wherein the CAR includes a transmembrane domain.
43. A method of genetically-modifying a cell to have reduced CD33 expression including exposing the cell to an effective amount of a base-editing system that reduces CD33 expression.
44. A method of embodiment 43, wherein the base-editing system is a cytosine-base editing system that replaces guanine/cytosine base pair with an adenine/thymine base pair.
45. A method of embodiment 44, wherein the cytosine-base editing system includes cytosine deaminase.
46. A method of embodiment 43, wherein the base-editing system includes a CRISPR-based nuclease, a zinc finger nuclease or a transcription activator like effector nuclease.
47. A method of embodiment 46, wherein the nuclease has nickase function.
48. A method of embodiment 46, wherein the CRISPR-based nuclease includes Cas9.
49. A method of embodiment 43, wherein the base-editing system includes a DNA glycosylate inhibitor.
50. A method of embodiment 49, wherein the DNA glycosylate inhibitor includes a uracil DNA glycosylate inhibitor.
51. A method of embodiment 43, wherein the base-editing system is selected from BE1, BE2, BE3, HF-BE3, BE4, BE4max, BE4-GAM, YE1-BE3, EE-BE3, YE2-BE3, YEE-BE3, VQR-BE3, VRER-BE3, Sa-BE3, SA-BE4, SaBE4-Gam, SaKKH-BE3, Cas12a-BE, Target-AID, Target-AID-NG, xBE3, eA3A-BE3, A3A-BE3, and BE-PLUS.
52. A method of embodiment 43, wherein the base-editing system is BE4max and/or SaBE4-Gam.
53. A method of embodiment 43, wherein the genetic modification inactivates the intron1 splicing donor site of CD33.
54. A method of embodiment 43, wherein the genetic modification results in introduction of a stop codon within the CD33-coding sequence.
55. A method of embodiment 43, wherein the genetic modification results in introduction of a stop codon within exon 2 of the CD33-coding sequence.
56. A method of embodiment 43, wherein the cell is a hematopoietic stem and progenitor cell (HSPC).
57. A method of embodiment 43, wherein the cell is a CD34+CD45RA-CD90+ HSC.
58. A method of embodiment 43, wherein the cell is a therapeutic cell.
59. A method of embodiment 43, wherein the cell includes a genetic modification that results in inclusion of a therapeutic gene.
60. A method of embodiment 59, wherein the therapeutic gene is recited in one of embodiments 17, 18, 19, 20, or 21.
61. A method for treating a subject in need thereof with a pharmaceutical formulation of embodiment 23 including administering a therapeutically effective amount of the pharmaceutical formulation to the subject thereby treating the subject.
62. A method of embodiment 61, wherein the treating provides a therapeutically effective treatment against a primary immune deficiency.
63. A method of embodiment 61, wherein the treating provides a therapeutically effective treatment against a secondary immune deficiency.
64. A method of embodiment 61, wherein the treating provides a therapeutically effective treatment for a disorder including: FA, SCID, Pompe disease, Gaucher disease, Fabry disease, Mucopolysaccharidosis type I, familial apolipoprotein E deficiency and atherosclerosis (ApoE), viral infections, and cancer.
65. A method of embodiment 61, further including administering to the subject a CD33-targeting agent.
66. A method of embodiment 65, wherein the CD33-targeting agent includes an anti-CD33 antibody, an anti-CD33 immunotoxin, an anti-CD33 antibody-drug conjugate, an anti-CD33 antibody-radioisotope conjugate, an anti-CD33 bispecific antibody, an anti-CD33 bispecific immune cell engaging antibody, an anti-CD33 trispecific antibody, and/or an anti-CD33 chimeric antigen receptor (CAR) described in any of the preceding embodiments.

### Second Set of Exemplary Embodiments

1. A method of selectively protecting a cell from an anti-CD33 therapeutic, the method including contacting the cell with a base editing system including a base editing enzyme and a guide RNA (gRNA), wherein the base editing system inactivates expression of CD33.
2. The method of embodiment 1, wherein the contacting includes administering to a system or subject including the cell: a nucleic acid encoding the base editing enzyme and a nucleic acid encoding the gRNA; or the base editing enzyme and the gRNA.
3. The method of embodiment 2, wherein the system is an in vitro or ex vivo cell or cell culture.
4. A method of selectively protecting a cell of a human subject from an anti-CD33 agent, the method including: administering to a human subject a viral vector including a nucleic acid sequence encoding a base editing system including a base editing enzyme and a guide RNA (gRNA), wherein the base editing system inactivates expression of CD33; and administering to the human subject the anti-CD33 agent.
5. A population of cells including a first subpopulation expressing CD33 and a second subpopulation in which CD33 expression is inactivated, wherein one or more cells of the population include at least one base editing agent of a base editing system selected from a base editing enzyme and a guide RNA (gRNA), wherein the base editing system inactivates expression of CD33, optionally wherein CD33 expression is inactivated in at least 5%, 6%, 7%, 8%, 9%, 10%, 20%, 30%, 40%, or 50% of cells of the population.
6. A cell including a base editing system including a base editing enzyme and a guide RNA (g RNA), wherein the base editing system inactivates expression of CD33.
7. A base editing system that inactivates CD33 in a cell, the base editing system including a base editing enzyme and a guide RNA (gRNA).
8. A kit including a base editing enzyme of a base editing system and a guide RNA (gRNA) of a base editing system, wherein the base editing system inactivates expression of CD33, optionally further including an anti-CD33 agent and/or instructions for inactivation of CD33 in one or more cells.
9. The method, population, cell, system, or kit of any one of embodiments 1-8, wherein the base editing system is engineered to cause a genetic modification that inactivates CD33, wherein the inactivating genetic modification includes a genetic modification at a splicing site of a nucleic acid encoding CD33, optionally wherein the splicing site is a splicing donor site or a splicing acceptor site, optionally wherein the splicing site is an intron 1 splicing donor site, an exon 2 splicing acceptor site, or an exon 3 splicing acceptor site of a nucleic acid encoding CD33.
10. The method, population, cell, system, or kit of any one of embodiments 1-8, wherein the base editing system is engineered to cause a genetic modification that inactivates CD33, wherein the inactivating genetic modification includes introduction of a stop codon within a nucleic acid encoding CD33.
11. The method, population, cell, system, or kit of embodiment 10, wherein the inactivating genetic modification includes introduction of a stop codon within exon 2 or exon 3 of a nucleic acid encoding CD33.
12. The method, population, cell, system, or kit of any one of embodiments 1-9, wherein the gRNA is engineered to cause a CD33-inactivating nucleic acid sequence modification selected from C to T at position 38 (G to A on forward strand, intron 1 splicing donor), C to T at position 481 (G to A on forward strand, intron 2 splicing donor), A to G at position 98 (exon2 splice acceptor), A to G at position 683 (exon3 splice acceptor), or A to G at position 1189 (exon4 splice acceptor) of CD33 (using SEQ ID NO: 15 as a reference).
13. The method, population, cell, system, or kit of any one of embodiments 1-12, wherein the gRNA is engineered to cause a CD33-inactivating nucleic acid sequence modification encoded by any of SEQ ID NOs: 4, 5, 19, 20, 21, and 22, optionally wherein the nucleic acid sequence modification is A to G at position −113 or A to G at position −175 of CD33.
14. The method, population, cell, system, or kit of any one of embodiments 1-13, wherein the gRNA has at least 80% sequence identity with a sequence selected from SEQ ID NOs: 4, 5, 19, 20, 21, and 22.
15. The method, population, cell, system, or kit of any one of embodiments 1-13, wherein the gRNA has at least 90% sequence identity with a sequence selected from SEQ ID NOs: 4, 5, 19, 20, 21, and 22.
16. The method, population, cell, system, or kit of any one of embodiments 1-15, wherein the base-editing system includes a cytosine base editing enzyme.
17. The method, population, cell, system, or kit of embodiment 16, wherein the gRNA is engineered to cause a CD33-inactivating nucleic acid sequence modification selected from C to T at position 38 (G to A on forward strand, intron 1 splicing donor), or C to T at position 481 (G to A on forward strand, intron 2 splicing donor) of CD33 (using SEQ ID NO: 15 as a reference).
18. The method, population, cell, system, or kit of embodiment 16 or 17, wherein the gRNA is engineered to cause a CD33-inactivating nucleic acid sequence modification encoded by any of SEQ ID NOs: 4, 5, 20, 21, and 22.
19. The method, population, cell, system, or kit of embodiment 16-18, wherein the gRNA has at least 80% sequence identity with a sequence selected from SEQ ID NOs: 4 and 5.
20. The method, population, cell, system, or kit of embodiment 16-19, wherein the gRNA has at least 90% sequence identity with a sequence selected from SEQ ID NOs: 4 and 5.
21. The method, population, cell, system, or kit of any one of embodiments 16-20, wherein the cytosine base-editing enzyme is selected from BEI, BE2, BE3, HF-BE3, BE4, BE4max, BE4-GAM, YE1-BE3, EE-BE3, YE2-BE3, YEE-BE3, VQR-BE3, VRER-BE3, Sa-BE3, SA-BE4, SaBE4-Gam, SaKKH-BE3, Cas12a-BE, Target-AID, Target-AID-NG, xBE3, eA3A-BE3, A3A-BE3, and BE-PLUS, optionally wherein the cytosine base-editing enzyme is BE4max and/or SaBE4-Gam.
22. The method, population, cell, system, or kit of any one of embodiments 1-15, wherein the base-editing system includes an adenine base editing enzyme.
23. The method, population, cell, system, or kit of embodiment 22, wherein the gRNA is engineered to cause a CD33-inactivating nucleic acid sequence modification selected from A to G at position 98 (exon2 splice acceptor), A to G at position 683 (exon3 splice acceptor), or A to G at position 1189 (exon4 splice acceptor) of CD33 (using SEQ ID NO: 15 as a reference).
24. The method, population, cell, system, or kit of embodiment 22 or 23, wherein the gRNA is engineered to cause a CD33-inactivating nucleic acid sequence modification encoded by any of SEQ ID NOs: 19, 20, 21, and 22.
25. The method, population, cell, system, or kit of embodiment 22-24, wherein the gRNA has at least 80% sequence identity with a sequence selected from SEQ ID NOs: 19, 20, 21, and 22.
26. The method, population, cell, system, or kit of embodiment 22-25, wherein the gRNA has at least 90% sequence identity with a sequence selected from SEQ ID NOs: 19, 20, 21, and 22.
27. The method, population, cell, system, or kit of any one of embodiments 22-26, wherein the adenine base editing enzyme is TadA*-dCas9, TadA-TadA*-Cas9, ABE7.9, ABE 6,3, ABE7.10, and/or ABE8e.
28. The method, population, cell, system, or kit of any one of embodiments 1-27, wherein the cell or cells are hematopoietic stem cells (HSCs).
29. The method, population, cell, system, or kit of any one of embodiments 1-27, wherein the cell or cells are hematopoietic stem and progenitor cells (HSPCs).
30. The method, population, cell, system, or kit of any one of embodiments 1-27, wherein the cell or cells are CD34+ HSCs and/or CD34+CD45RA-CD90+ HSCs.
31. The method, population, cell, system, or kit of any one of embodiments 1-30, wherein the base editing enzyme and/or gRNA are encoded by a vector. or synthesized in vitro
32. The method, population, cell, system, or kit of any one of embodiments 1-30, wherein the base editing enzyme and/or gRNA synthesized in vitro.
33. The method, population, cell, system, or kit of embodiment 31, wherein the vector is a viral vector, optionally wherein the viral vector is an adenoviral vector.
34. The method, population, cell, system, or kit embodiment 33 wherein the adenoviral vector is a helper dependent adenoviral vector.
35. The method, population, cell, system, or kit embodiment 33 or 34, wherein the adenoviral vector is a helper-dependent Ad35 viral vector.
36. The method, population, cell, system, or kit of embodiments 32-35, wherein the vector selectively targets HSCs or HSPCs.
37. The method, population, cell, system, or kit of any one of embodiments 32-36, wherein the vector further encodes a therapeutic polypeptide and/or further includes a therapeutic gene.
38. The method, population, cell, system, or kit of embodiment 37, wherein the therapeutic polypeptide is selected from a checkpoint inhibitor, a gene editing molecule, a chimeric antigen receptor that specifically binds a cellular antigen (e.g. a cancer antigen or a viral antigen), a T-cell receptor that specifically binds a cellular antigen (e.g. a cancer antigen or a viral antigen), γ-globin; soluble CD40; CTLA; Fas L; antibodies to CD4, CD5, CD7, CD52, etc.; antibodies to IL1, IL2, IL6; an antibody to TCR specifically present on autoreactive T cells; IL4; IL10; IL12; IL13; ID Ra, sIL1RI, sIL1RII; sTNFRI; sTNFRII; antibodies to TNF; P53, PTPN22, and DRB1*1501/DQB1*0602; globin family genes; WAS; phox; dystrophin; pyruvate kinase (PK); CLN3; ABCD1; arylsulfatase A (ARSA); SFTPB; SFTPC; NLX2.1; ABCA3; GATA1; ribosomal protein genes; TERC; CFTR; LRRK2; PARK2; PARK7; PINK1; SNCA; PSEN1; PSEN2; APP; SOD1; TDP43; FUS; ubiquilin 2; C9ORF72, von Willebrand factor (VWF), FI, FII, FV, FVII, factor VIII (FVIII), FIX, FX, FXI, and/or FXIII, optionally wherein the therapeutic polypeptide is selected from FVIII and/or FIX.
39. The method, population, cell, system, or kit of embodiment 37, wherein the therapeutic gene is selected from FancA, FancB, FancC, FancD1, FancD2, FancE, FancF, FancG, Fancl, FancJ, FancL, FancM, FancN, FancO, FancP, FancQ, FancR, FancS, FancT, FancU, FancV, FancW, γC, JAK3, IL7RA, RAG1, RAG2, DCLRE1C, PRKDC, LIG4, NHEJ1, CD3D, CD3E, CD3Z, CD3G, PTPRC, ZAP70, LCK, AK2, ADA, PNP, WHN, CHD7, ORAI1, STIM1, CORO1A, CIITA, RFXANK, RFX5, RFXAP, RMRP, DKC1, TERT, TINF2, DCLRE1B, SLC46A1, ABLI, AKT1, APC, ARSB, BCL11A, BLC1, BLC6, BRCA1, BRCA2, BRIP1, C46, CAS9, C-CAM, CBFAI, CBL, CCR5, CD19, CDA, C-MYC, CRE, CSCR4, CSFIR, CTS-I, CYB5R3, DCC, DHFR, DLL1, DMD, EGFR, ERBA, ERBB, EBRB2, ETSI, ETS2, ETV6, FCC, FGR, FOX, FUSI, FYN, GALNS, GLB1, GNS, GUSB, HBB, HBD, HBE1, HBG1, HBG2, HCR, HGSNAT, HOXB4, HRAS, HYAL1, ICAM-1, iCaspase, IDUA, IDS, JUN, KLF4, KRAS, LYN, MCC, MDM2, MGMT, MLL, MMACI, MYB, MEN-I, MEN-II, MYC, NAGLU, NANOG, NF-1, NF-2, NKX2.1, NOTCH, OCT4, p16, p2I, p27, p57, p73, PALB2, RAD51C, ras, at least one of RPL3 through RPL40, RPLPO, RPLP1, RPLP2, at least one of RPS2 through RPS30, RPSA, SGSH, SLX4, SOX2, VHL, and/or WT-I.
40. A population of cells including a first subpopulation expressing CD33 and a second subpopulation in which CD33 expression is inactivated, wherein one or more cells includes an inactivated CD33 gene including a nucleic acid sequence according to one or more of SEQ ID NOs: SEQ ID NOs: 4, 5, 19, 20, 21, or 22, optionally wherein CD33 expression is inactivated in at least 5%, 6%, 7%, 8%, 9%, 10%, 20%, 30%, 40%, or 50% of cells of the population, optionally wherein the cells are HSCs, HSPCs, CD34+ HSCs, and/or CD34+CD45RA-CD90+ HSCs.
41. A cell including an inactivated CD33 gene including a nucleic acid sequence according to one or more of SEQ ID NOs: SEQ ID NOs: 4, 5, 19, 20, 21, or 22, optionally wherein the cell is an HSC, HSPC, CD34+ HSC, and/or CD34+CD45RA-CD90+ HSC.
42. The method, population, cell, system, or kit of any one of embodiments 1-41, wherein one or more of the cell or cells is contacted with an anti-CD33 agent.
43. A pharmaceutical formulation including a population, cell, system, or kit of any one of embodiments 5-42 and a pharmaceutically acceptable carrier.
44. A method of treating a subject in need thereof, the method including administering to the subject a population, cell, system, kit, or pharmaceutical formulation of any one of embodiments 5-43.
45. The method of embodiment 44, wherein the method includes administering to the subject an anti-CD33 agent.
46. The method of embodiment 44 or 45, wherein the subject is need of treatment for a primary immune deficiency, a secondary immune deficiency, or a disorder selected from FA, SCID, Pompe disease, Gaucher disease, Fabry disease, Mucopolysaccharidosis type I, familial apolipoprotein E deficiency and atherosclerosis (ApoE), viral infections, and/or cancer.
47. The method of embodiment 44 or 45, wherein the subject is need of treatment for a hematology condition, optionally wherein the hematology condition is a platelet disorder, a bone marrow failure condition, a red cell disorder, an autoimmune hematology, a primary immunodeficiency, or an inborn error of metabolism.
48. The method of embodiment 44 or 45, wherein the subject is need of treatment for a hematology condition selected from Bernard-Soulier syndrome, Glanzmann thrombasthenia, Diamond-Blackfan anemia, pyruvate kinase deficiency, acquired thrombotic thrombocytopenic purpura (aTTP), congenital thrombotic thrombocytopenic purpura (cTTP), Wiskott-Aldrich syndrome (WAS), Severe combined immunodeficiency due to adenosine deaminase deficiency (ADA-SCID), X-linked severe combined immunodeficiency (SCID-X1), DOCK 8 deficiency, major histocompatibility complex class II deficiency (MHC-II), CD40/CD40L deficiency, hereditary hemochromatosis, and phenylketonuria (PKU).
49. The method, population, cell, system, or kit of any one of embodiments 42-48, wherein the anti-CD33 agent includes an anti-CD33 antibody, an anti-CD33 immunotoxin, an anti-CD33 antibody-drug conjugate, an anti-CD33 antibody-radioisotope conjugate, an anti-CD33 bispecific antibody, an anti-CD33 bispecific immune cell engaging antibody, an anti-CD33 trispecific antibody, an anti-CD33 chimeric antigen receptor (CAR) with one or more binding domains, hp67.6, lintuzumab, SGN-CD33A, and/or AMG 330.
50. The method, population, cell, system, or kit of any one of embodiments 42-48, wherein the anti-CD33 agent includes a binding domain derived from hp67.6, lintuzumab, SGN-CD33A, and/or AMG 330, wherein the anti-CD33 agent includes one or more, or all, CDRs of hp67.6, lintuzumab, SGN-CD33A, and/or AMG 330, and/or wherein the anti-CD33 agent includes a bispecific antibody including a combination of binding variable chains or a binding CDR combination of hp67.6, lintuzumab, SGN-CD33A, and/or AMG 330.
51. The method, population, cell, system, or kit of any one of embodiments 42-48, wherein the anti-CD33 agent includes an antibody-drug conjugate or an antibody-radioisotope conjugate wherein the drug or radioisotope are selected from taxol, taxane, cytochalasin B, gramicidin D, ethidium bromide, emetine, mitomycin, etoposide, tenoposide, vincristine, vinblastine, colchicin, doxorubicin, daunorubicin, dihydroxy anthracinedione, mitoxantrone, mithramycin, maytansinoid, dolastatin, auristatin, calicheamicin, pyrrolobenzodiazepine, nemorubicin PNU-159682, anthracycline, vinca alkaloid, trichothecene, CC1065, camptothecin, elinafide, actinomycin D, 1-dehydrotestosterone, glucocorticoids, procaine, tetracaine, lidocaine, propranolol, puromycin, ricin, CC-1065, duocarmycin, diphtheria toxin, snake venom, cobra venom, mistletoe lectin, modeccin, pokeweed antiviral protein, saporin, Bryodin 1, bouganin, gelonin, Pseudomonas exotoxin, iodine-131, indium-111, yttrium-90, lutetium-177, astatine-211, bismuth-212, and/or bismuth-213 and/or wherein the antibody-drug conjugate includes GO.
52. The method, population, cell, system, or kit of any one of embodiments 42-48, wherein the CD33-targeting agent includes a bispecific antibody including at least one binding domain that activates an immune cell, optionally wherein the immune cell is a T-cell, natural killer (NK) cell, or a macrophage and//or wherein the binding domain that activates an immune cell binds CD3, CD28, CD8, NKG2D, CD8, CD16, KIR2DL4, KIR2DS1, KIR2DS2, KIR3DS1, NKG2C, NKG2E, NKG2D, NKp30, NKp44, NKp46, NKp80, DNAM-1, CD11 b, CD11 c, CD64, CD68, CD119, CD163, CD206, CD209, F4/80, IFGR2, Toll-like receptors 1-9, IL-4Ra, or MARCO, optionally wherein the binding domains of the bispecific antibody are joined through a linker.
53. The method, population, cell, system, or kit of any one of embodiments 42-48, wherein the CD33-targeting agent includes a chimeric antigen receptor (CAR) including a binding domain that specifically binds CD33.
54. The method, population, cell, system, or kit of embodiment 53, wherein: the CAR includes an effector domain selected from 4-1BB, CD3ε, CD3δ, CD3, CD27, CD28, CD79A, CD79B, CARD11, DAP10, FcRα, FcRβ, FcRγ, Fyn, HVEM, ICOS, Lck, LAGS, LAT, LRP, NOTCH1, Wnt, NKG2D, OX40, ROR2, Ryk, SLAMF1, Slp76, pTα, TCRα, TCRβ, TRIM, Zap70, PTCH2, or any combination thereof; the CAR includes a cytoplasmic signaling sequence derived from CD3 zeta, FcR gamma, CD3 gamma, CD3 delta, CD3 epsilon, CDS, CD22, CD79a, CD79b, or CD66d; the CAR includes an intracellular signaling domain and a costimulatory signaling region, optionally wherein the costimulatory signaling region includes the intracellular domain of CD27, CD28, 4-1 BB, OX40, CD30, CD40, lymphocyte function-associated antigen-1, CD2, CD7, LIGHT, NKG2C, or B7-H3; the CAR includes a spacer region; and/or the CAR includes a transmembrane domain.
55. The method, population, cell, system, or kit of any one of embodiments 42-54s wherein the anti-CD33 agent includes a linker.

### EXAMPLES

CD33 is a polypeptide known to be expressed by, among other cell types, HSCs. The present disclosure recognizes that engineering of HSCs in general presents a valuable avenue for treatment of many medical conditions. In various such treatments, HSCs can be administered as a therapeutic, and in certain embodiments can be engineered in vitro, ex vivo, or in vivo to introduce a therapeutic genetic modification that addresses a medical condition of interest. The present disclosure includes that CD33 inactivation in HSCs can provide a means of advantageously selecting or selectively protecting therapeutic cells. As provided herein, base editing of CD33 can inactivate CD33 expression by HSCs, whereby upon administration of an anti-CD33 agent that selectively kills, or otherwise inhibits growth and/or proliferation of CD33-expressing cells, CD33-inactivated HSCs are selected for. The present Examples demonstrate that inactivation of CD33 in HSCs by base editing provides an efficient, effective, and advantageous method of producing CD33-inactivated HSCs. Inactivation of CD33 by base editing, in contrast with CRISPR editing, can be multiplexed with additional concurrent base edits in individual cells, e.g., further therapeutic base edits including in other genes and optionally on other chromosomes. Inactivation of CD33 by base editing, in contrast with CRISPR editing, produces a population of cells capable of efficient engraftment and/or survival and/or differentiation in vivo.

### Example 1: Inactivation of CD33 by Base Editors

The present Example demonstrates that base editors of the present disclosure efficiently inactivate CD33 in target cells and can concurrently edit multiple targets in the same cell, e.g., a CD33 inactivating edit and a further therapeutic edit.

FIG. 1 provides exemplary polypeptide sequences of CD33 (through the transmembrane domain, but lacking the cytoplasmic domain) from Macaca fascicularis (SEQ ID NO: 1), Homo sapiens (SEQ ID NO: 2), and Mus musculus (SEQ ID NO: 3), aligned and annotated. The amino acid sequence of a full length human CD33 polypeptide is shown in SEQ ID NO: 14; additional full length sequences of representative CD33 proteins are shown in SEQ ID NO: 169 (Macaca mulatta), SEQ ID NO: 170 (Macaca fascicularis), and SEQ ID NO: 171 (Mus musculus).

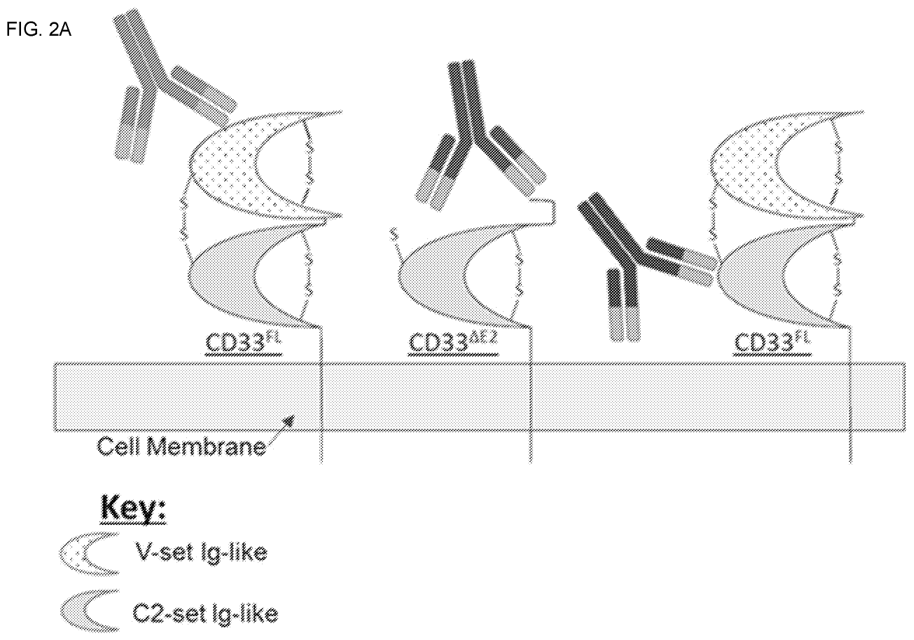

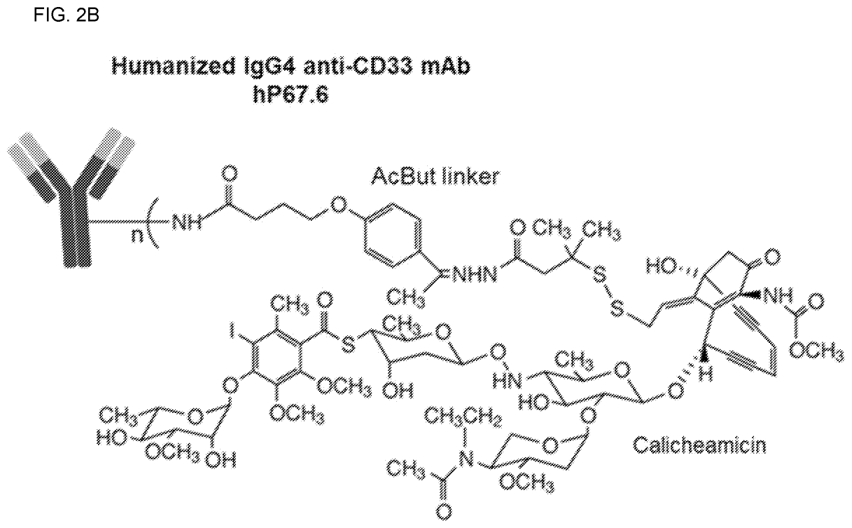

The amino acid sequence of a truncated CD33 polypeptide (CD33^{ΔE2}) including an engineered deletion of E2, which includes functional deletion of a V-set Ig-like domain of CD33 with which many anti-CD33 agents bind, is shown in SEQ ID NO: 17. Binding of anti-CD33 antibodies to the V-set Ig-like domain, C2-set Ig-like domain, or an engineered tag is illustrated in FIG. 2A. A schematic of a particular anti-CD33 antibody-drug conjugate, gemtuzumab ozogamicin (GO), is provided in FIG. 2B.

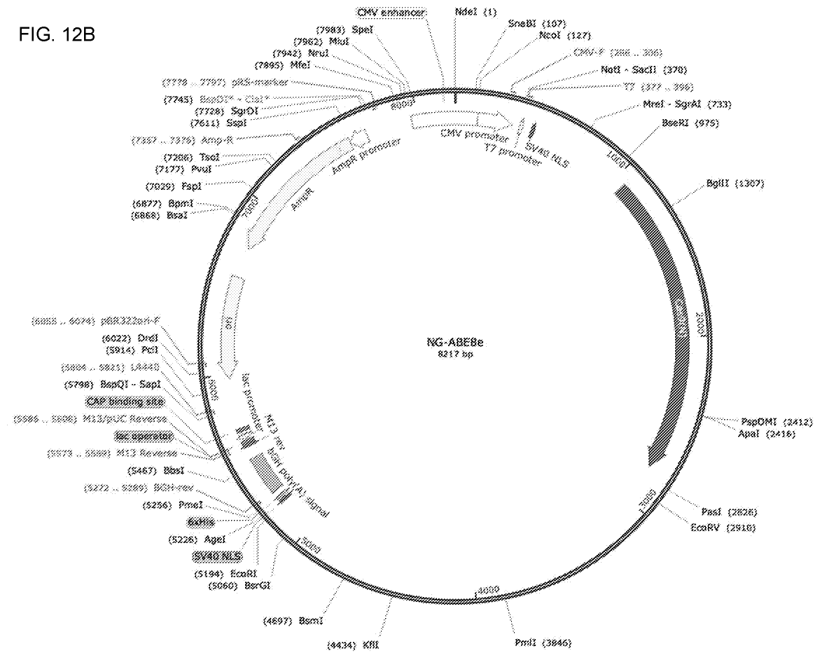

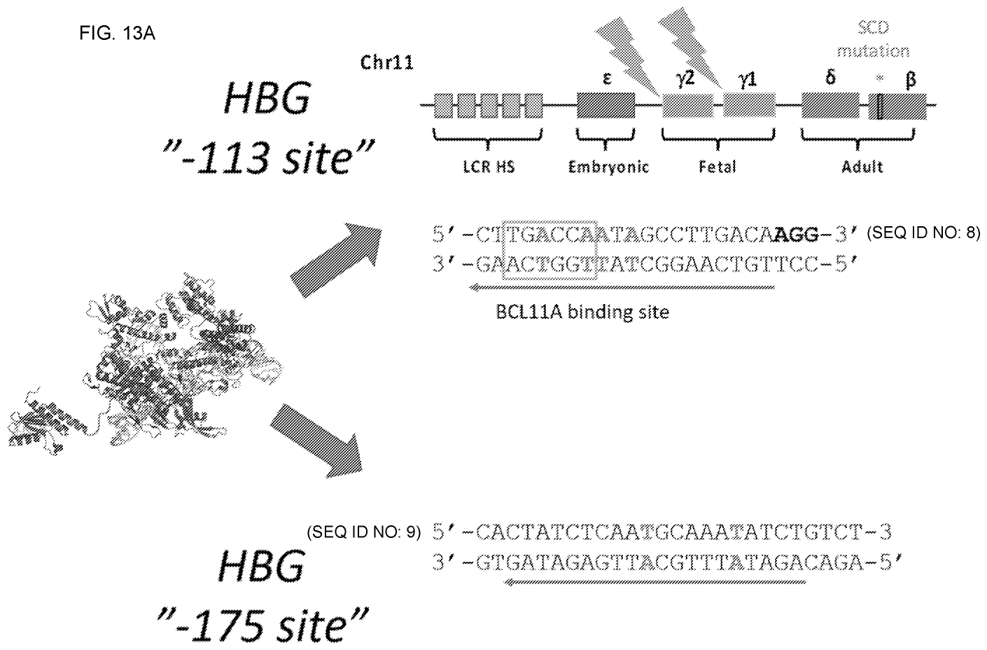

In the present Example, base editor enzymes were delivered to human or NHP cells in the form of messenger RNA (mRNA) and stored as frozen aliquots in a −20° C. freezer; or in the form of protein purified using immobilized metal affinity chromatography followed by cation exchange chromatography. FIGS. 12A and 12B are schematic drawings of the ABE8e (FIG. 12A; Addgene #138489; SEQ ID NO: 6) and the ABE8e-NG (FIG. 12B; Addgene #138491; SEQ ID NO: 7) plasmids, from which ABE8e was expressed. The development of ABE8e, and of these two plasmids, is described in Richter et al. (Nat. Biotech. 38(7):883-891, 2020).

Base editor mRNA was delivered to target cells together with guide RNAs (gRNAs) synthesized to include 2′-O-methyl analogs at each of the first three 5′ and the first three 3′ terminal RNA residues and to include 3′ phosphorothioate internucleotide linkages at each of the first three 5′ and the first three 3′ terminal RNA linkages. These modifications reduce the susceptibility of the gRNA to degradation in cells.

gRNAs were custom-ordered from Synthego (Redwood City, Calif.), shipped as lyophilized materials, resuspended in purified water upon arrival and stored as frozen aliquots in a -80 QC freezer. Target editing sites in the gamma globin (HBG) genes were selected within the promoter region to introduce sequence modifications at position -113 and/or at position -175; modification from A to G at these target editing site nucleotides (via deamination using ABE8e) reduces or prevents binding of a repressor (BCL11A) and thereby will result in increased expression of the fetal hemoglobin genes. Table 11 provides the sequences of three ABE8e gRNA targets were utilized; the positions targeted for edits are shown bold.

TABLE 11

| | | | SEQ ID | |
|---|---|---|---|---|
| Name | Sequence* | type | NO | Purpose |
| HBG-113 | CUUGACCAAUAGCCUUGACA | ABE | 137 | relevant for |
| HBG-175 | AGAUAUUUGCAUUGAGAUAG | ABE | 138 | hemoglobinopathies |
| | | | | therapy |
| CD33E2 splice | CCCCACAGGGGCCCUGGCUA | ABE | 19 | CD33 knockdown |

*Each gRNA was synthesized in vitro with the groups 2′-O-methyl analogs and 3′ phosphorothioate internucleotide linkages at the first three 5′ and 3′ terminal RNA residues.

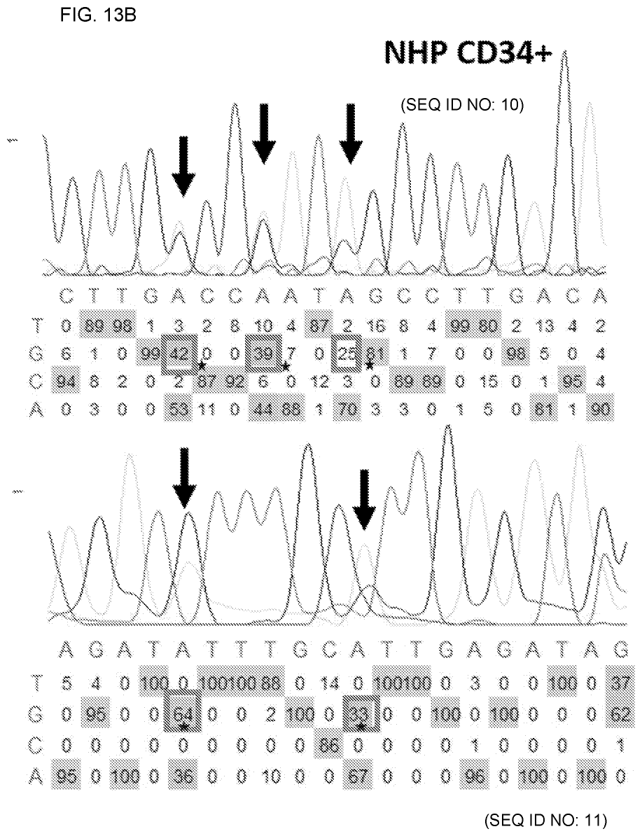

FIGS. 13A-13B illustrate this system for targeting of two HBG promoter target editing sites with ABE8e in nonhuman primate NHP CD34+ cells for the reactivation of fetal hemoglobin. FIG. 13A is a schematic of HBG target sites.

Ribonucleoproteins (RNP) were formed by combining 180 pmol of ABE8e protein with 1800 pmol of gRNA at room temperature for 10 min and used for the electroporation of 3 million cells in 2-mm cuvettes following the manufacturer's instructions (Harvard Apparatus, Holliston, Mass.).

RNP or mRNA encoding BE were delivered to CD34+ cells via electroporation, which minimizes toxicity and temporally limits exposure to the mutagenic editing reagent.

For colony forming cell (CFC) assays, 1000 to 1200 sorted cells were seeded into 3.5 ml ColonyGEL 1402 (ReachBio). Hematopoietic colonies were scored after 12 to 14 days. Arising colonies were identified as colony-forming unit (CFU) granulocyte (CFU-G), CFU macrophage (CFU-M), CFU granulocyte-macrophage (CFU-GM), and burst-forming unit-erythrocyte (BFU-E). Colonies consisting of erythroid and myeloid cells were scored as CFU-MIX.

Editing at both CD33-inactivating and therapeutic target sites was quantified by next generation sequencing (NGS) using Illumina barcoded, 2×150 base pair (bp) pair-end MiSeq primers for complete sequencing (Illumina) or by EditR analysis (Kluesner et al., CRISPR J. 1(3):239-250, 2018; PMID: 31021262) of bulk cell populations and also of clonal hematopoietic colonies to determine the rates of simultaneous edits within the same cell.

Results:

Data demonstrate efficient modification by base editing of nucleotides at target editing sites in CD34+ cells to inactivate CD33, increase gamma globin expression, or both. Concurrent multiplexed base editing of multiple targets is also demonstrated for CD33 inactivation and a further therapeutic edit, in particular base editing to increase expression of gamma globin.

FIG. 13B is a pair of result tables showing editing efficiency measured by EditR analysis (Kluesner et al., CRISPR J. 1(3):239-250, 2018; PMID: 31021262). As shown in FIG. 13B, additional nucleotides within the editing target site of the tested gRNAs are also modified. Arrows show the position of edits; starred (★) boxes show frequencies of the targeted edits.

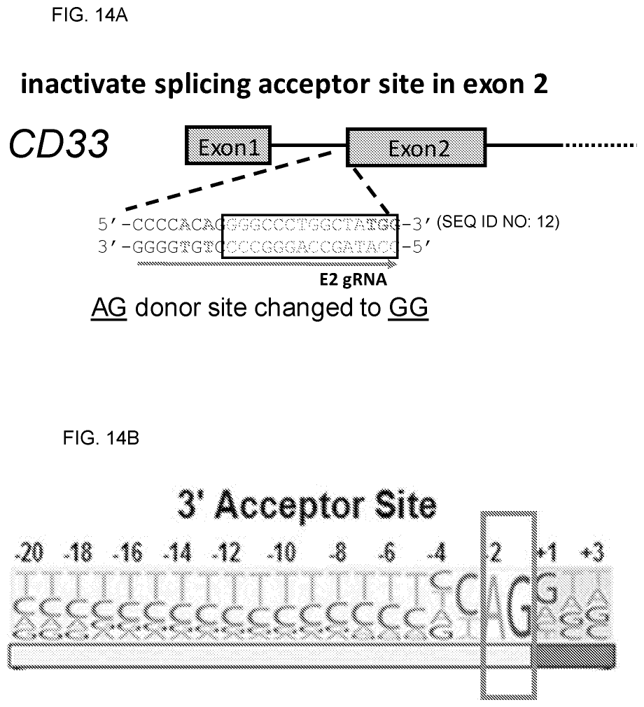

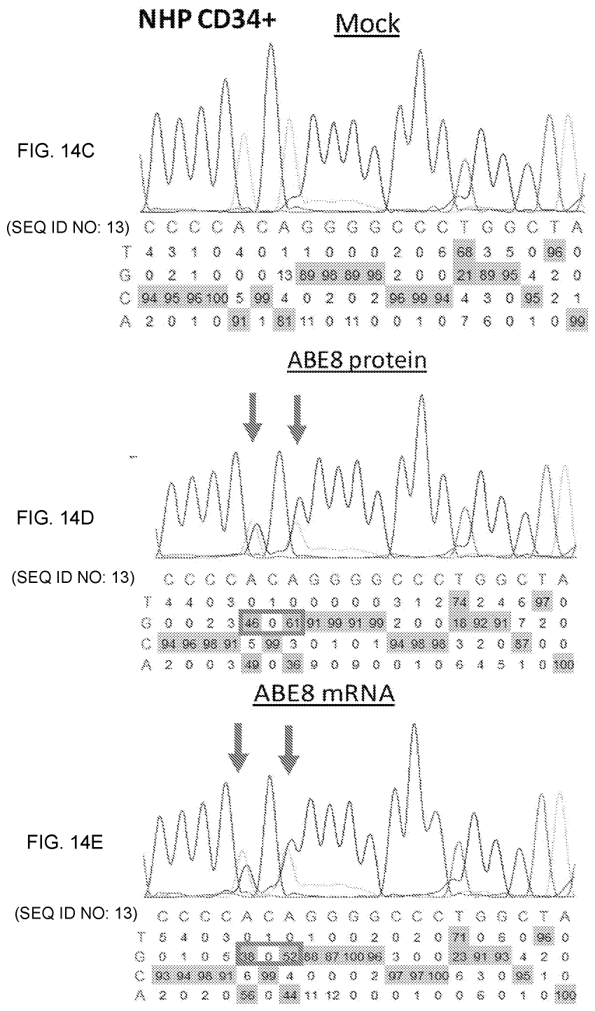

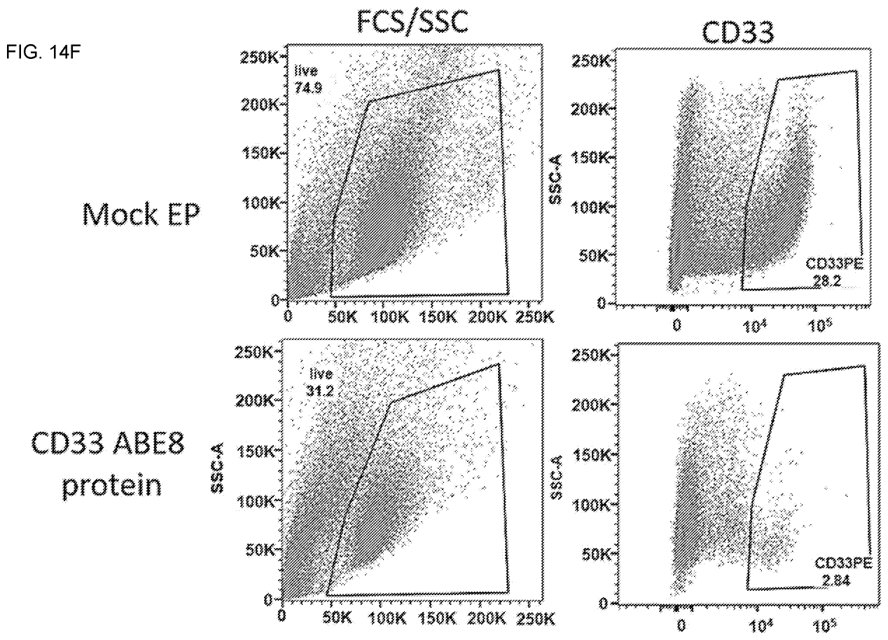

FIGS. 14A-14F show efficient CD33 knockdown with ABE8e. FIG. 14A shows targeting of CD33 splicing site (exon2 acceptor site) with ABE8e in NHP CD34+ cells. FIG. 14B is an illustration of conservation of the 3′ acceptor site; the AG that are boxed are universal, and therefore an excellent target for editing. The splicing acceptor site in exon 2 is inactivated, by editing the AG donor site to GG. FIG. 14C-14E show the editing efficiency of the CD33 target site, measured by EditR, in non-human primate (NHP) CD34+ cells mock treated (FIG. 14C), treated with ABE8e protein (FIG. 14D) or with ABE8e mRNA (FIG. 14E). Arrows show the position of edits; starred (★) boxes show frequencies. FIG. 14F shows flow cytometry analysis of CD33 surface expression in NHP CD34+ cells at six days post-treatment. (T234 rhesus CD34+ (6 days post EP)). The editing efficiency achieved using this system is both specific and unexpectedly high; the efficiency is on a par with rates that might be achieved with CRISPR editing.

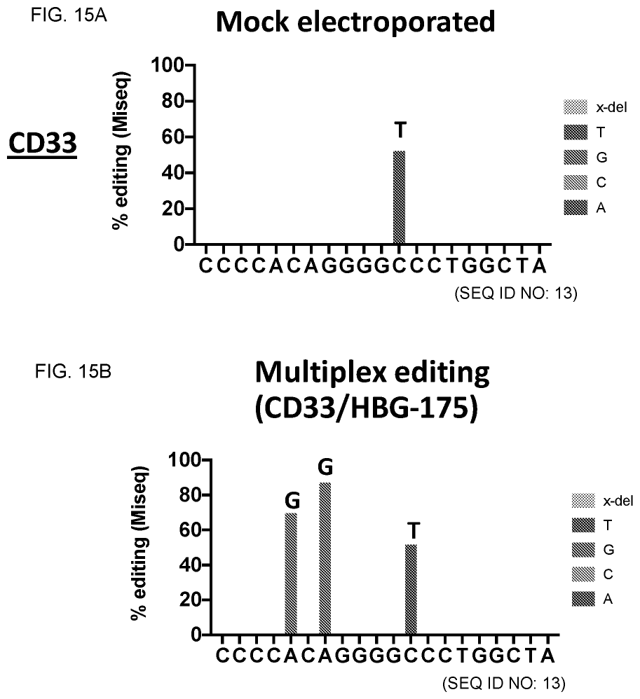

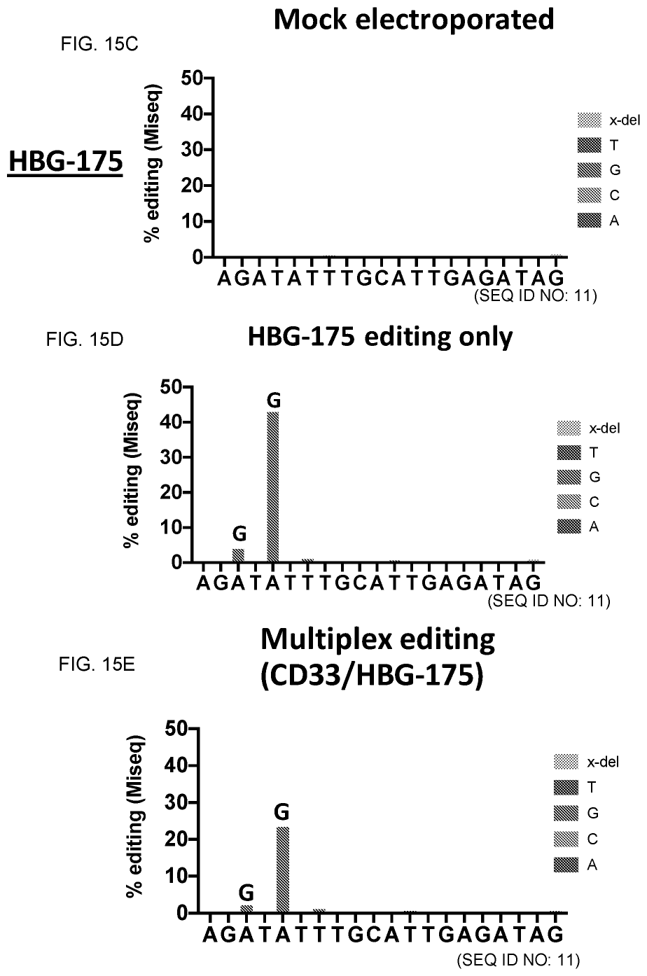

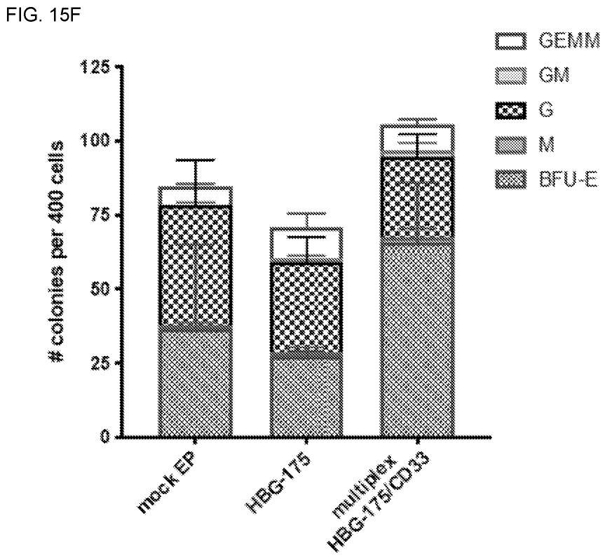

FIGS. 15A, 15B are a pair of graphs illustrating multiplex ABE8e HBG/CD33 editing in human fetal liver (FL) CD34+ cells. Cells were edited with ABE8e mRNA and single guide RNA (sgRNA) targeting each of the CD33 and HBG-175 sites. Editing efficiency was measured by next generation sequencing (NGS) at the CD33 (FIGS. 15A and 15B) or HBG-175 (FIGS. 15C-15E) sites. Graphs show editing frequency at each nucleotide within the target site. The positional difference in editing efficiency, such as is shown in FIGS. 15B, 15D, and 15E, was unexpected. For CD33 in FIG. 15A, the C-to-T variation is due to a natural variant not induced by editing. FIG. 15F is a bar graph showing there is minimal impact of multiplex editing on the capacity of human FLCD34+ cells to differentiate, using a colony forming analysis system. Cells were plated in a colony forming assay to evaluate the impact of editing on HSC multilineage differentiation. The graph shows the number of each type of differentiated cell (GEMM: granulocyte, erythroid macrophage, and megakaryocyte, GM: granulocyte-macrophage, G: granulocyte, M: macrophage, or BFU-E: burst-forming unit-erythrocyte) counted in colonies formed from plating edited 400 cells.

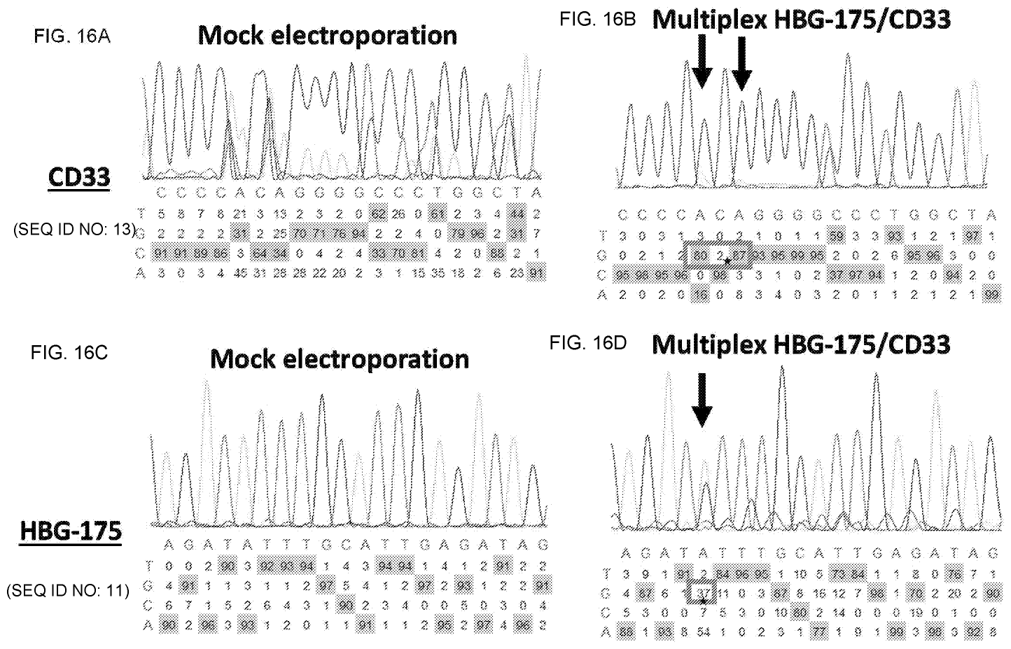

FIGS. 16A-16D is a series of graphs illustrating multiplex ABE8e HBG/CD33 editing in human mobilized peripheral blood (mPB) CD34+ cells. CD34+ cell culture. Human CD34+ cells were harvested and enriched from G-CSF mobilized peripheral blood (PB). For enrichment of CD34+ cells, red cells were lysed in ammonium chloride lysis buffer, and white blood cells were incubated for 20 min with the 12.8 immunoglobulin M anti-CD34 antibody and then washed and incubated for another 20 min with magnetic-activated cell-sorting anti-immunoglobulin M microbeads (Miltenyi Biotec). The cell suspension was run through magnetic columns enriching for CD34+ cell fractions with a purity of 60 to 80% confirmed by flow cytometry. Enriched CD34+ cells were cultured in stemspan serum-free expansion medium II (SFEM II) (STEMCELL Technologies) supplemented with penicillin and streptomycin (100 U/ml; Gibco by Life Technologies), stem cell factor (PeproTech), thrombopoietin (PeproTech), and Fms-related tyrosine kinase 3 ligand (Miltenyi Biotec) (100 ng/ml for each cytokine). Cells were edited with ABE8e mRNA and single guide RNA (sgRNA) targeting each of the CD33 and HBG-175 sites. Editing efficiency was measured by EditR at the CD33 or HBG-175 sites. Arrows show the position of edits; starred (★) boxes show editing frequencies.

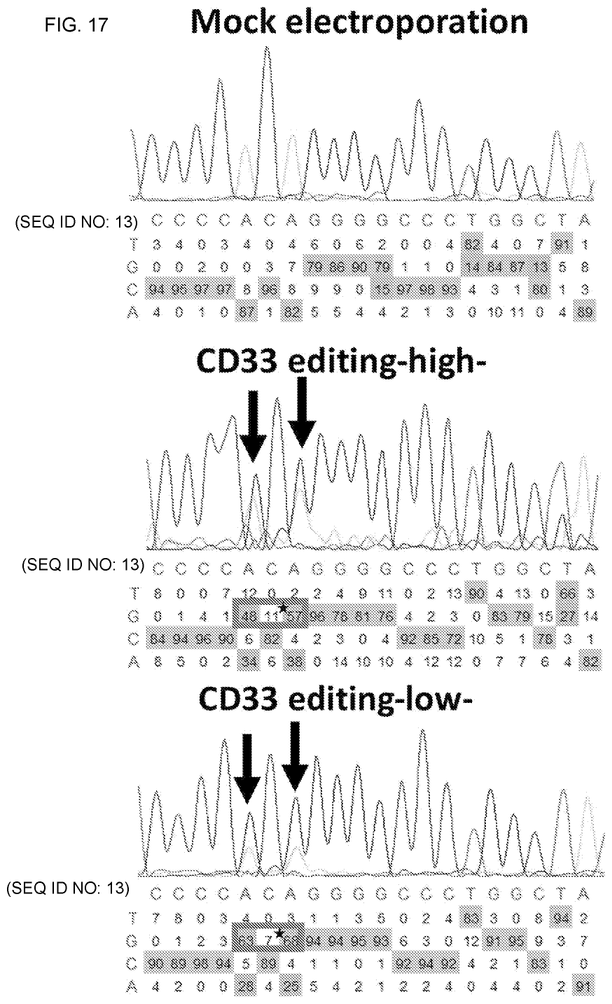

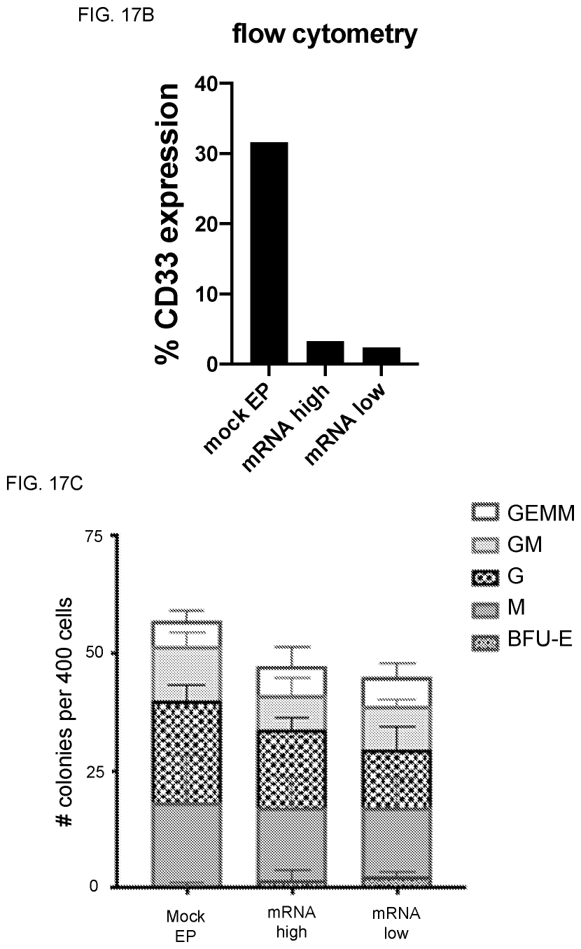

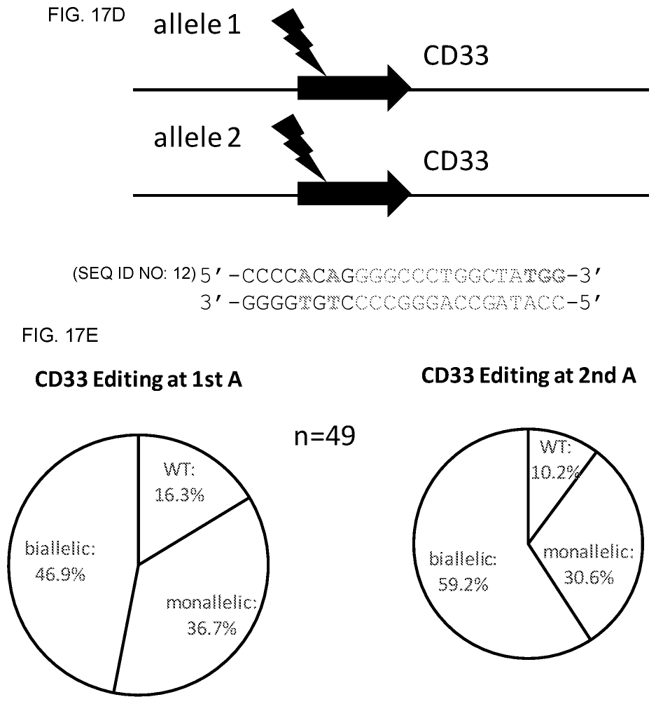

FIGS. 17A-17E illustrate ABE8e CD33 editing in NHP CD34+ cells. Cells were edited with two different concentrations (high and low) of ABE8e mRNA and single guide RNA (sgRNA) targeting CD33. FIG. 17A is three panels showing editing efficiency measured by EditR. Arrows show the position of edits, and starred (★) boxes show editing frequencies. FIG. 17B is a bar graph showing percentage of CD33 expression in the same edited cells, measured by flow cytometry analysis. FIG. 17C is a bar graphs showing there is minimal impact of ABE8e editing using either high or low mRNA on the capacity of NHP CD34+ cells to differentiate, measured using a colony forming analysis system. Cells were plated in a colony forming assay to evaluate the impact of editing on HSC multilineage differentiation, as in FIG. 15B. FIG. 17D is a schematic drawing of mono- vs. bi-allelic CD33 editing. FIG. 17E is a pair of pie graphs showing the percentage of mono- vs. bi-allelic CD33 editing frequencies (in treated NHP CD34+ cells) measured in single colonies (n=49) at either targeted adenine.

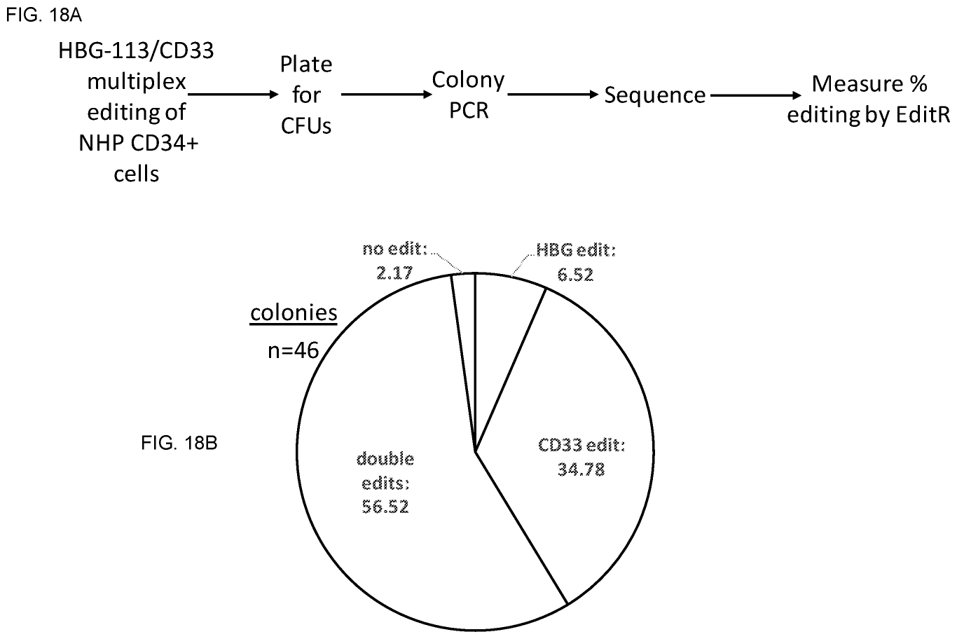

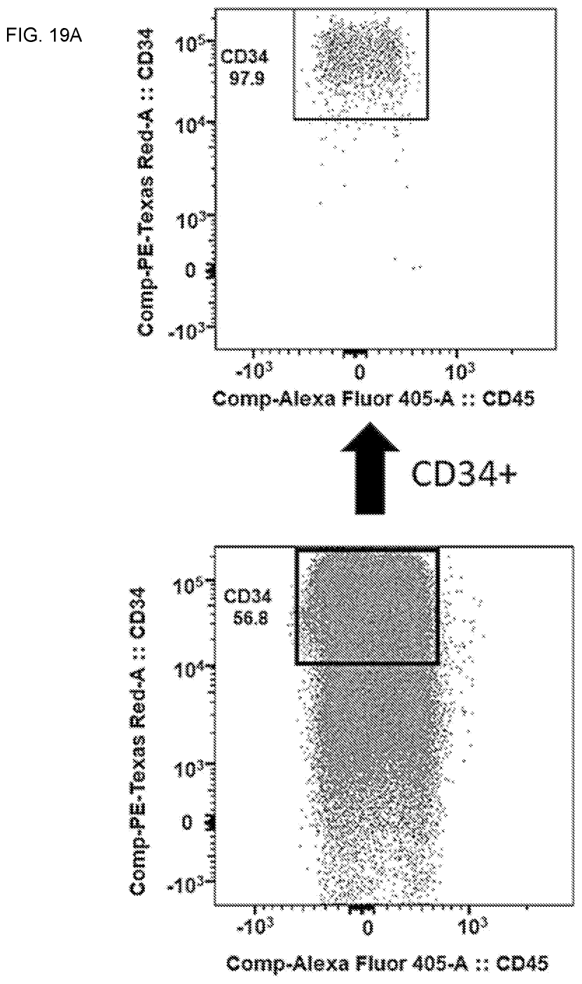

FIGS. 18A-18B illustrate multiplex ABE8e HBG/CD33 editing in NHP CD34+ cells and analysis of single- vs. double-edits at a single cell level. FIG. 18A is an outline of the experimental procedure. FIG. 18B is a pie graph showing the frequency of single- vs. double-edits in derived colonies (n=46) from treated CD34+ cells. Over half of the colonies show editing at both targets.

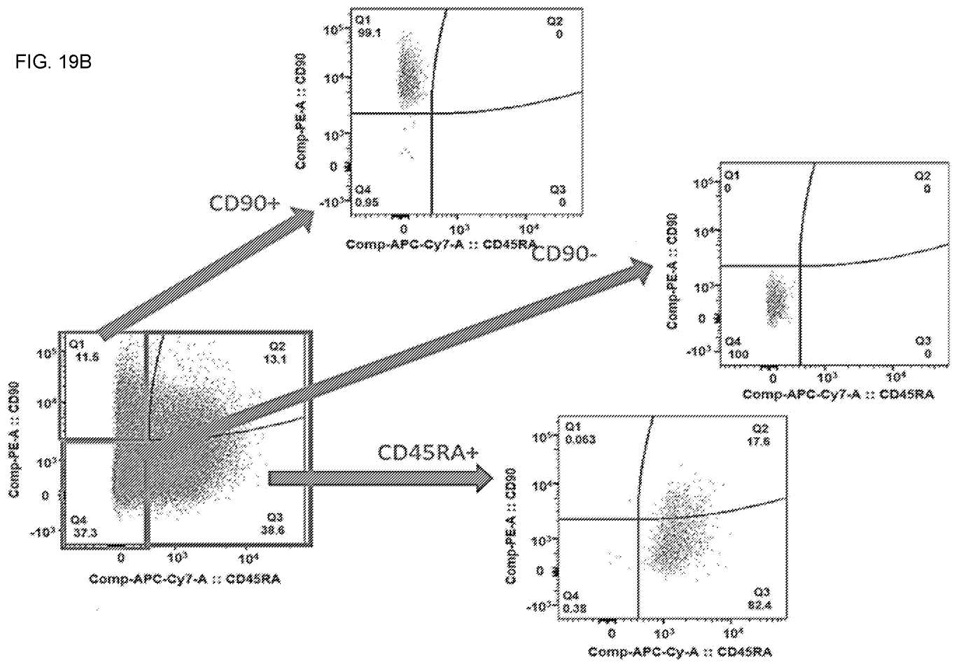

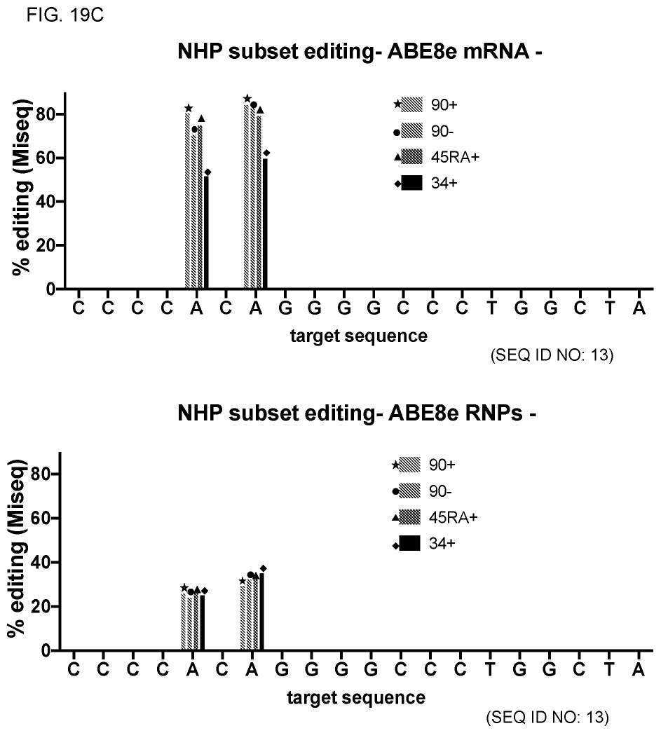

FIGS. 19A-19C show ABE8e CD33 editing in NHP HSPC subsets. NHP CD34+ cells (bottom panel, FIG. 19A) were treated with ABE8e mRNA or RNPs targeting CD33 and subsequently sorted for the different HSPC subpopulations: CD34+ (FIG. 19A, top panel), CD90+, CD90- and CD45RA+(FIG. 19B). Validation of the purity of the sorting experiment is shown. CD33 editing efficiency in the different subpopulations from FIG. 19A-19B, measured by NGS, is shown in cells treated with ABE8e mRNA (FIG. 19C, top panel) or RNPs (FIG. 19C, bottom panel).

### Example 2: Efficient Engraftment of Base Edited Cells in Mice

While CD33 inactivation is a promising strategy for selection and/or selective protection of cells of therapeutic value, various embodiments require that the edited cells are capable of in vivo engraftment. The present disclosure includes the surprising observation that base edited cells display remarkably high in vivo engraftment as compared to CRISPR edited cells.

Engraftment of human CD34+ cells in the mouse xenotransplant model. For in vivo assessment of engineered HSPCs, NOD.Cg-PrkdcscidII2rgtm1Wjl/Szj (NOD SCID gamma/, NSG) neonate mice were infused with 5.0×10⁵ fetal liver human CD34+ cells or adult mice were infused with 1.0×10⁶ PB human CD34+ cells. Peripheral blood and tissue samples collected and processed as previously described (Godwin et al., Leukemia 34(9):2479-2483, 2020; PMID: 32071429). Flow cytometry staining was performed with human CD45-PerCP (Clone 2D1), mouse CD45.1/CD45.2-V500 (Clone 30-F11), CD3-FITC or -APC (Clone UCHT1), CD4-V450 (Clone RPA-T4), CD2O-PE (Clone 2H7), CD14-APC or -PE-Cy7 (Clone M5E2), CD34-APC (Clone 581) (all from BD Biosciences, San Jose, Calif.), and CD33-PE (Clone AC104.3E3, Miltenyi Biotec). Engraftment, multilineage differentiation and in vivo editing efficiency of treated HSPCs were tracked longitudinally in peripheral blood and tissues.

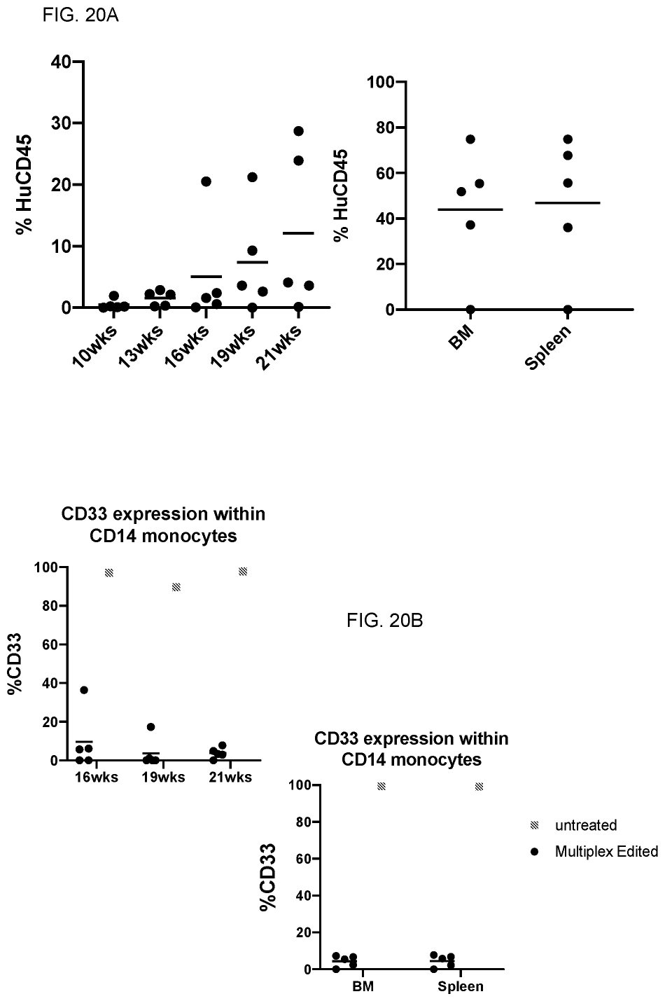

FIGS. 20A, 20B illustrate engraftment of multiplex edited ABE8e HBG/CD33 FL human CD34+ cells in immunodeficient mice. Cells edited for both HBG and CD33 using ADE8e (as described for FIG. 19) were administered to immunodeficient mice, and the ability of the multiplex edited cells to home to bone marrow and differentiate was examined. FIG. 20A is a pair of graphs showing longitudinal tracking of human cell engraftment based on human CD45+ flow cytometry staining from peripheral blood over 21 weeks (left) or from spleen and bone marrow of transplanted mice at the time of necropsy (right). FIG. 20B is a pair of graphs showing persistence of CD33 knockdown after engraftment. Longitudinal tracking of CD33 expression from peripheral blood over 21 weeks (left) or from spleen and bone marrow of transplanted mice at the time of necropsy (right); untreated cells are solid squares, and multiplex edited cells are solid circles.

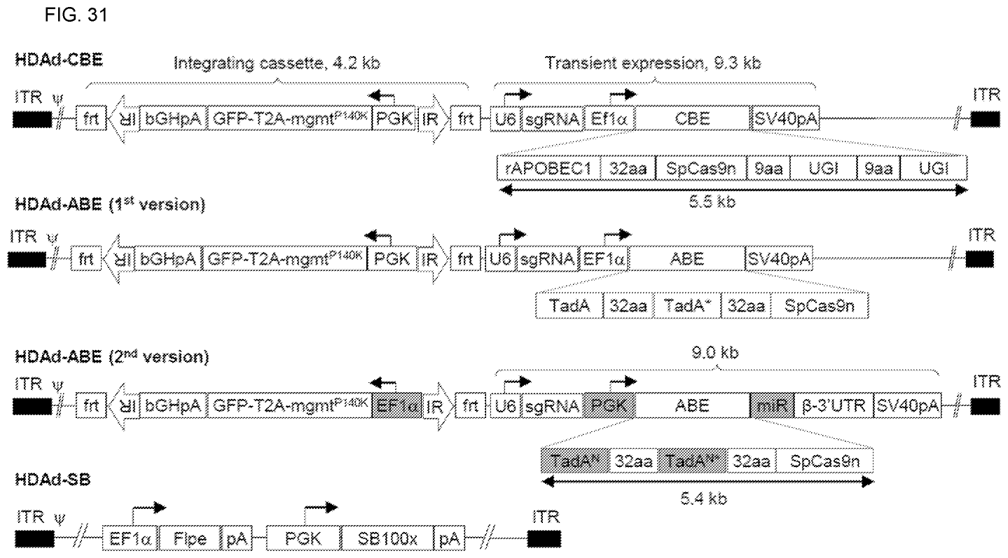

The total length of base editor genes plus regulatory elements exceeds 8 kb and the payload capacity of lentiviral or adeno-associated virus vectors. HDAd5/35++ vectors that can package genomes of ˜32 kb can address this problem. In a first version, the BE enzyme (rAPOBEC1-nCas9-2×UGI for CBE; 2xTadA-nCas9 for ABE) driven by an EF1a promoter and the sgRNA driven by a human U6 promoter were cloned into the HDAd plasmid pHCA. An mgmt/GFP cassette flanked by frt and transposon sites was also cloned into the vector to mediate selection of transduced cells by O⁶BG/BCNU (FIG. 31). Notably, the BE components were placed outside the SB100× transposon, only allowing for their transient expression while, at the same time, maintaining integrated expression of the mgmt/GFP cassette upon co-delivery with an HDAd-SB vector expressing SB100× transposase/flippase (Wang et al., Mol Ther Methods Clin Dev. 1:14057, 2015). Although the yield per 2-liter spinner culture was relatively low (1×10¹² viral particles or vp on average), it was possible to rescue all four CBE vectors. This is in contrast to HDAd-CRISPR vectors that are not rescuable without mechanisms that regulate nuclease expression (Li et al., Cancer Res. 80(3):549-560, 2020). The results suggested that DSB-free BE systems may be less toxic to the HDAd producer cells (116 cells) than CRISPR/Cas9. For the ABE vectors, the virus genome appeared rearranged and no distinct HDAd band was observed after ultracentrifugation in CsCl gradients. Since the major difference between ABE and CBE vectors is the deaminase domain, it was likely that the two 594 bp TadA-32aa repeats in ABE vectors were the elements causing recombination and rearrangements within the HDAd genome. To address this problem, the following modifications to the original version of the ABE vectors were made: i) the sequence repetitiveness between the two TadA-32aa repeats was reduced by alternative codon usage; ii) A PGK promoter was used to drive the BE enzyme expression. While being highly active in HSPCs33, the PGK promoter exhibits lower activity in 116 producer cells than EF1a34, thereby reducing potential TadA-associated adverse effects; and iii) a miR183/218-based gene regulation system was utilized to further suppress BE expression in 116 cells while allowing it in HSPCs32 (FIG. 31). This second version of ABE constructs with this optimized design led to successful rescue of two HDAd-ABE viruses with an average yield of 3.3×10¹² vp/spinner, which is within the normal yield range.

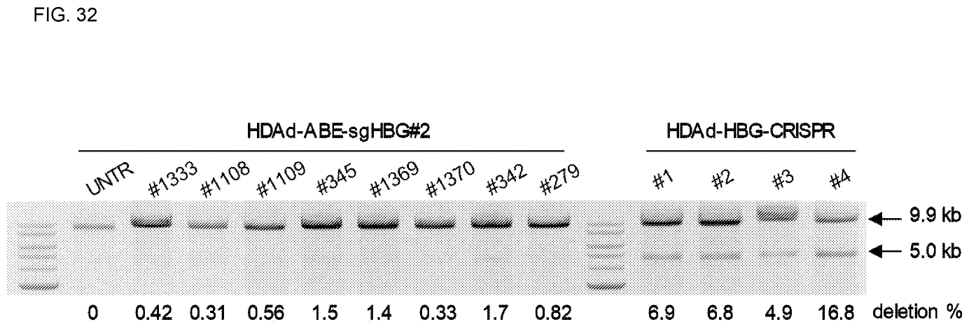

A potential problem with DSB-depending gene editing strategies is large genomic deletions/rearrangements (Kosicki et al., Nat Biotechnol. 36(8):765-771, 2018). This side effect become more pronounced in targeting the HBG1/2 promoters. Due to the two identical targets in the HBG1 and HBG2 regions, we and others have reported that CRISPR/Cas9 editing led to the 4.9 kb intergenic deletion (Traxler et al., Nat Med 22(9):987-990, 2016; Li et al., Blood 131(26):2915-2928, 2018). As a result, the whole HBG2 gene was removed. Therefore, we interrogated this genomic deletion by a semi-quantitative PCR described previously (Li et al., Blood 131(26):2915-2928, 2018). It is noteworthy that, by establishing a standard curve, this detection approach can adequately measure the percentage of deletion without overestimation (resulting from preferential PCR amplification of the shorter 5.0 kb band). For comparison, mouse samples treated side-by-side with a CRISPR/Cas9 vector (HDAd-HBG-CRISPR) targeting the HBG1/2 promoters (Li et al., Blood 131(26):2915-2928, 2018) were included. The average 4.9 kb deletion in the HDAd-BE-sgHBG #2-treated mice was 0.8% (FIG. 32). In some mice, it was barely detectable. This was, on average, ten-fold lower than that in HDAd-HBG-CRISPR treated samples. See also Example 10 in related application No. PCT/US2020/040756, which is incorporated herein by reference.

Engraftment and survival of edited HSCs in vivo is unexpectedly superior for HSCs in which a target editing site is modified by base editing (in this experiment, the AncBE4max system; Koblan et al., Nat Biotechnol. 36(9):843-846, 2018) as compared to HSCs in which a target editing site is inactivated by CRISPR where both were delivered via viral expression vector. Engraftment studies were conducted by transplanting CD34+ cells transduced with Ad5/35++ adenoviral vector encoding an ABE system (including a guide RNA referred to as HBG #2 and having the sequence CTTGACCAATAGCCTTGACA (SEQ ID NO: 10) for a target site edit of TGACCA, -113 A>G) or a CRISPR editing system including an HBG-specific sgRNA into sublethally irradiated NOD-scid IL2rγnull (NSG) mice carrying a 248 kb of the human β-globin locus (β-YAC mice) including the HBG #2 target editing site. See FIG. 31. Controls included untransduced mice, and mice transduced with a helper dependent Ad5/35++ empty vector control.

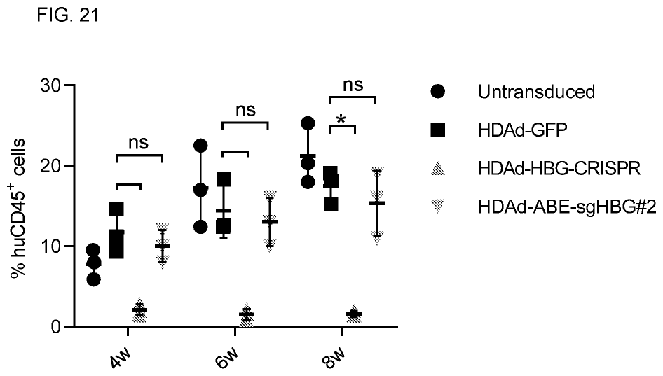

Results showed that unexpectedly HDAd-BE-sgHBG #2 transduction led to similar level of engraftment (measured by huCD45 expression) as the HDAd-GFP treatment (FIG. 21), whereas HDAd-HBG-CRISPR transduction largely deprived CD34+ cells of their engraftment capability, in line with previous findings (Li et al., Mol Ther Methods Clin Dev. 9:390-401, 2018). Importantly, the editing level was maintained in engrafted cells harvested at week 8 after transplantation. These data suggest no evident cytotoxicity from HDAd-ABE-sgHBG #2 transduction in human HSPCs. See also Example 11 in related application No. PCT/US2020/040756, which is incorporated herein by reference.

The present disclosure provides that the unexpected differential in HSC survival or proliferation in vivo demonstrated in FIG. 21 is the result of differential genotoxicity and cytotoxicity in HSCs. A major concern with current genome-editing technologies using CRISPR/Cas9 is that they introduce double-stranded DNA breaks (DSBs), which may be detrimental to host cells by causing unwanted large fragment deletion and p53-dependent DNA damage responses. Base editors are capable of installing precise nucleotide mutations at targeted genomic loci and present the advantage of avoiding DSBs. This study shows that a functional feature of HSCs that is critical to various embodiments, namely the survival and/or proliferation (e.g., engraftment) in sub-lethally irradiated NSG mice, is not affected by a BE but is dramatically reduced after transduction of human CD34+ cells with CRISPR/Cas9 expressing vector. While the general phenomenon of CRISPR editing system genotoxicity and cytotoxicity were known, and it was further known that base editing systems are characterized by reduced genotoxicity and cytotoxicity, the present disclosure demonstrates the unexpectedly great impact of this difference on survival and/or proliferation of HSCs. This unexpected differential disclosed herein is in one or more respects particular to HSCs and/or survival and/or proliferation of HSCs in vivo.

The difference in engraftment between CRISPR- and BE-modified HSCs is more pronounced after delivery using an expression vector (such as Ad5/35++ delivery exemplified here) as compared to delivery via RNP or mRNA electroporation likely because of the longer time period during which the editor is being expressed in the HSCs (a week or more with expression vector-based delivery, compared to 2-3 days with RNP or mRNA electroporation). The longer exposure to an expressed editing system increases chances for off-target effect and toxicity, which is more severe with CRISPR.

Discussion

In this Example, the editing efficiency and toxicity of delivery by ABE8e mRNA versus delivery by ABE8e purified protein in CD34+ HSPCs (human and NHPs) were compared and substantially better results were found with mRNA delivery.

### Example 3: In Vivo Selection of CD33-Inactivated Hematopoietic Stem Cells after CD33 Base Editing in Mice Treated with an Anti-CD33 Agent

The present Example further confirms efficient and effective inactivation of CD33 by base editing and efficient and effective survival and/or proliferation of cells in vivo (e.g., during engraftment following transplantation into a recipient). Data of the present Example further demonstrate selection of CD33-inactivated cells in vivo by administration of an anti-CD33 agent.

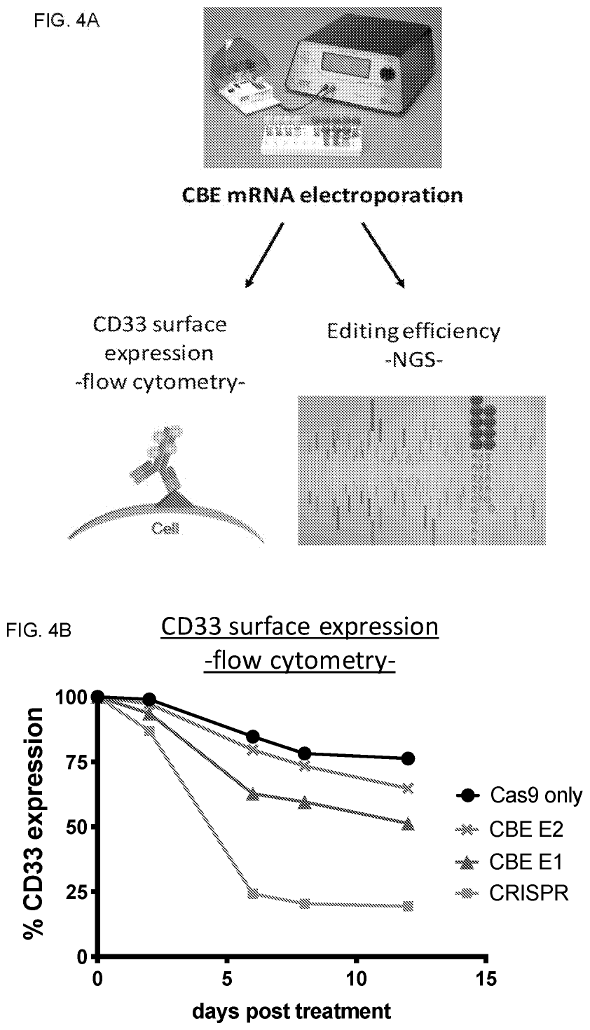

In the present Example, CD33 was inactivated by a cytidine base editor (CBE) system that converts C nucleotides to T nucleotides. Embodiments of this process are exemplified in FIGS. 3A-3B. Particular CD33 modifications inactivating intron1 splicing donor site and/or introducing a stop codon into exon 2 are also provided, together with the utilized gRNA sequences (FIG. 3B). These gRNA sequences (SEQ ID NOs: 4 and 5) were used to modify HSCs in vitro by CBE mRNA introduced through electroporation (FIG. 4A). As shown in FIG. 3B, E1 gRNA inactivates intron 1 splicing donor site and E2 gRNA introduces a stop codon in exon 2. Experiments were carried out using methods described in Example 1.

CD33 surface expression measured by flow cytometry with a labeled anti-CD33 antibody and next generation sequencing (NGS) were used to determine the efficiency of CD33 inactivation by gene editing in HSCs (FIGS. 4A and 4B). Flow cytometry results shown in FIG. 4B confirm inactivation of CD33 expression by a base editing system including the E1 or E2 gRNA (FIG. 3B) relative to Cas9 only reference at all measured time points. CD33 inactivation was also shown to be achieved by a CRISPR/Cas9 system engineered for CD33 inactivation by an E2 deletion. Inactivation of the intron 1 splicing donor site by base editing caused a greater decrease in percent CD33 expression detected by flow cytometry than introduction of a stop codon in exon 2 by base editing.

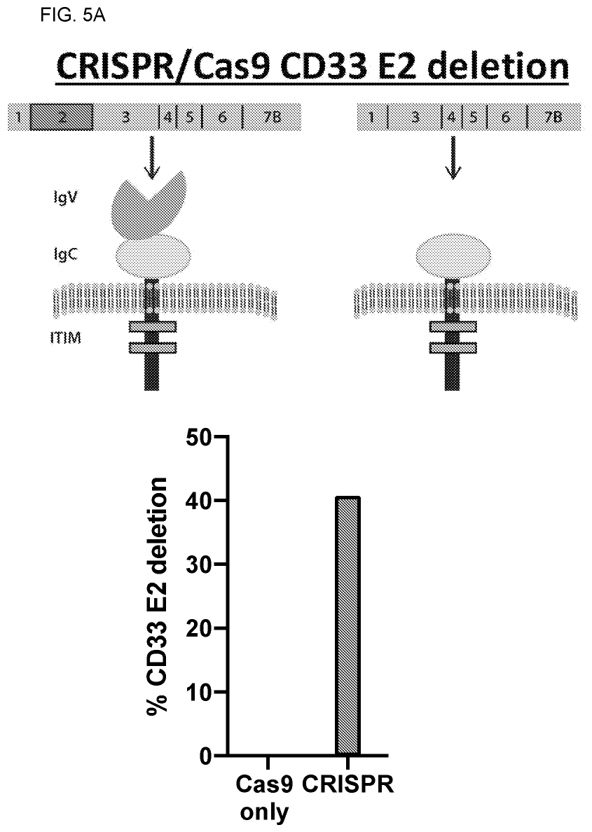

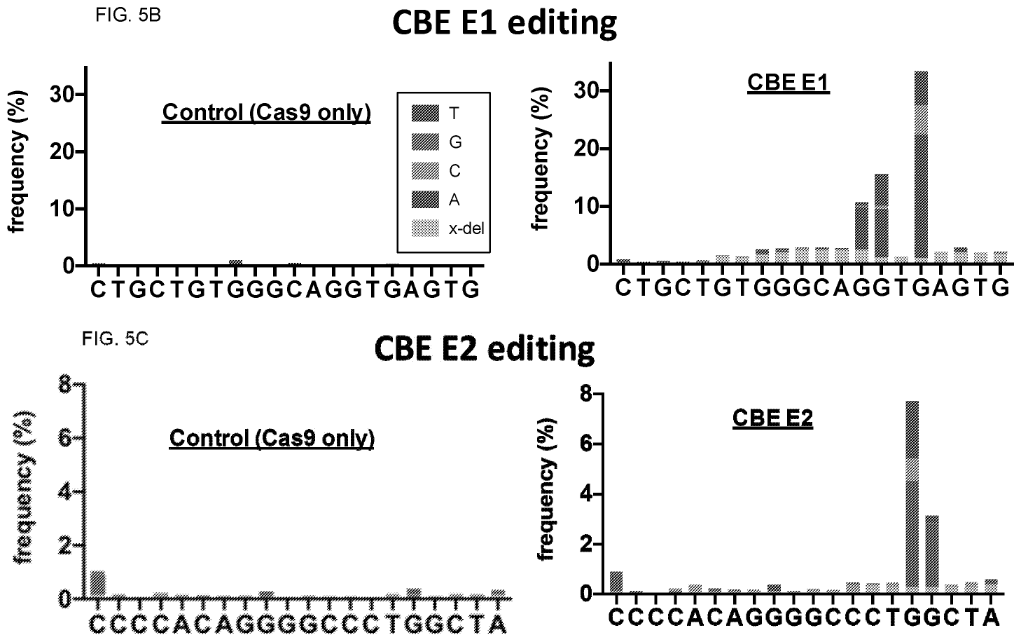

Percentage CD33 E2 deletion (FIG. 5A), CBE1 editing (FIG. 5B), and CBE2 editing (FIG. 5C) were measured in treated human fetal liver (FL) CD34+ cells, as compared to a Cas9 only control. CBE1 gRNA for inaction of the intron 1 splicing donor site caused a greater frequency of base editing than the CBE2 gRNA for introduction of a stop codon in exon 2.

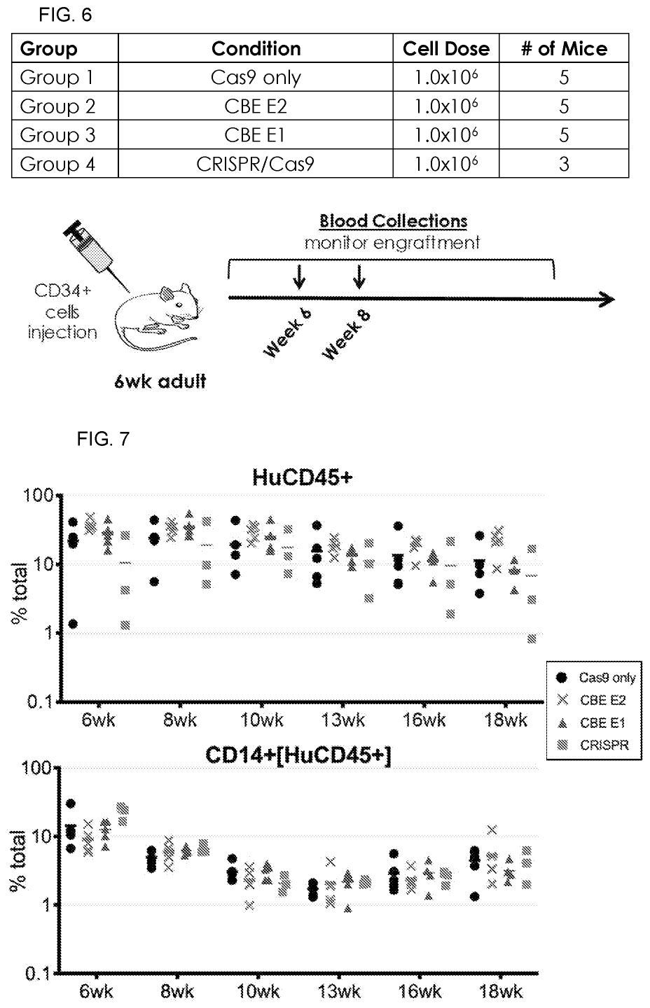

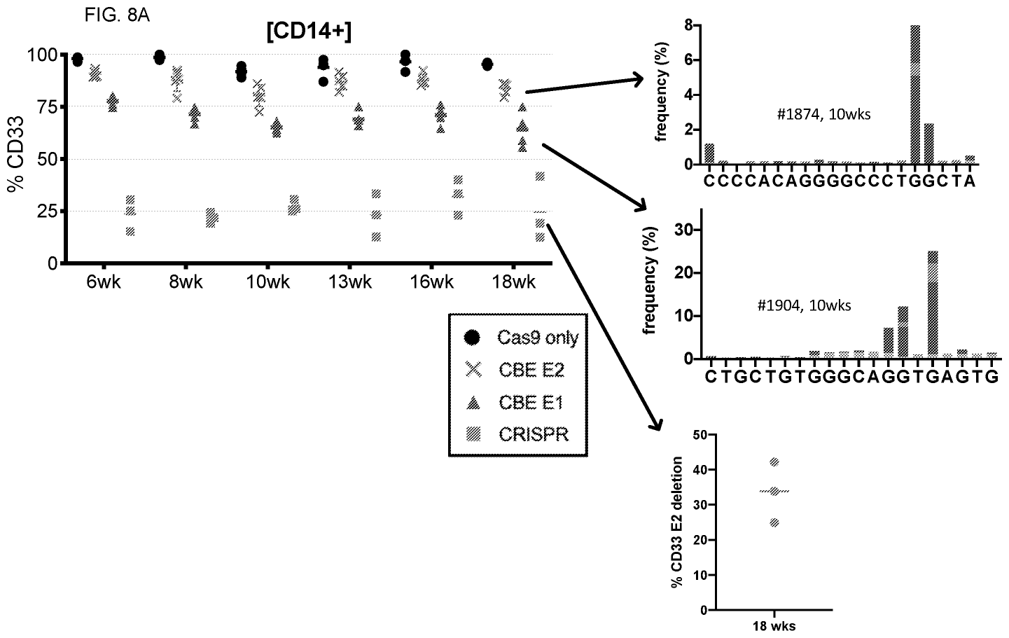

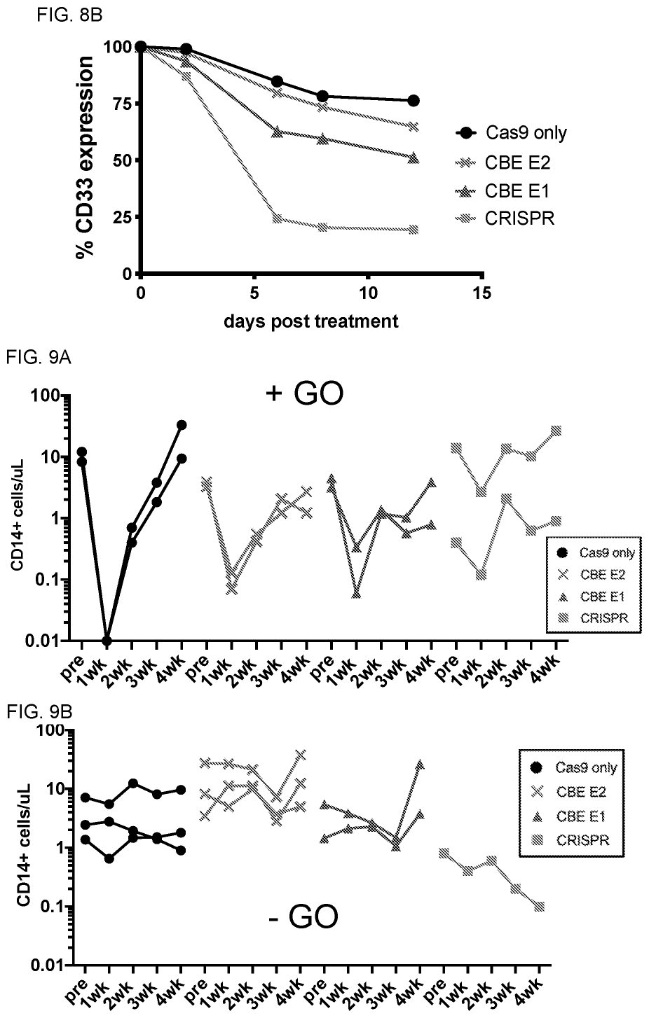

Engineered HSCs of the present Example were further transplanted by injection into mice and engraftment was monitored for 18 weeks (FIGS. 6 and 7). Measurements of total engraftment shown in FIG. 7 demonstrate that CBE-edited CD33-inactivated HSCs display normal engraftment and differentiation. As measured over up to 18 weeks, decreased CD33 expression and/or frequency of CD33 nucleic acid sequence inactivation in CBE-edited CD33-inactivated HSCs persisted across the measured time points (FIGS. 8A-8B).

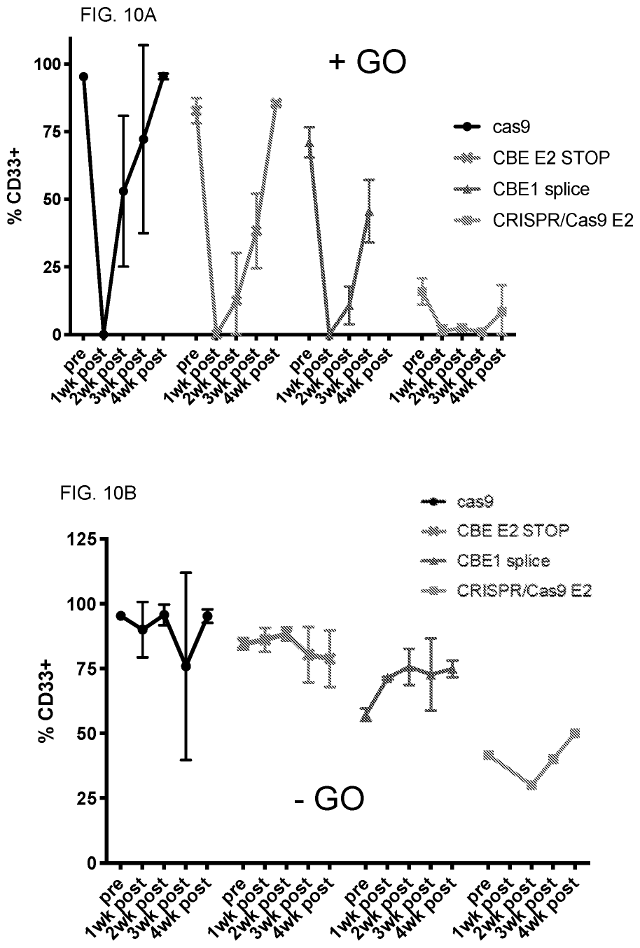

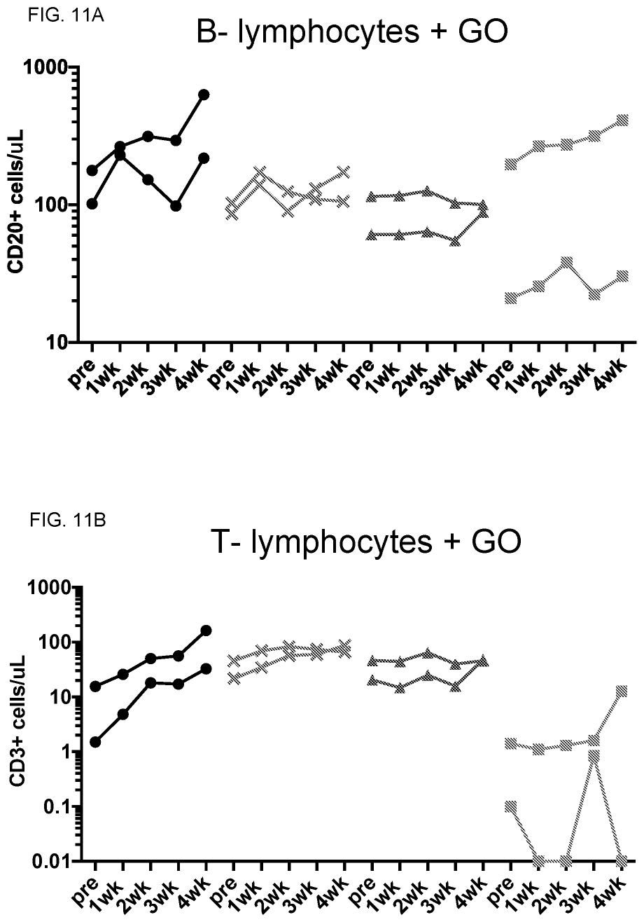

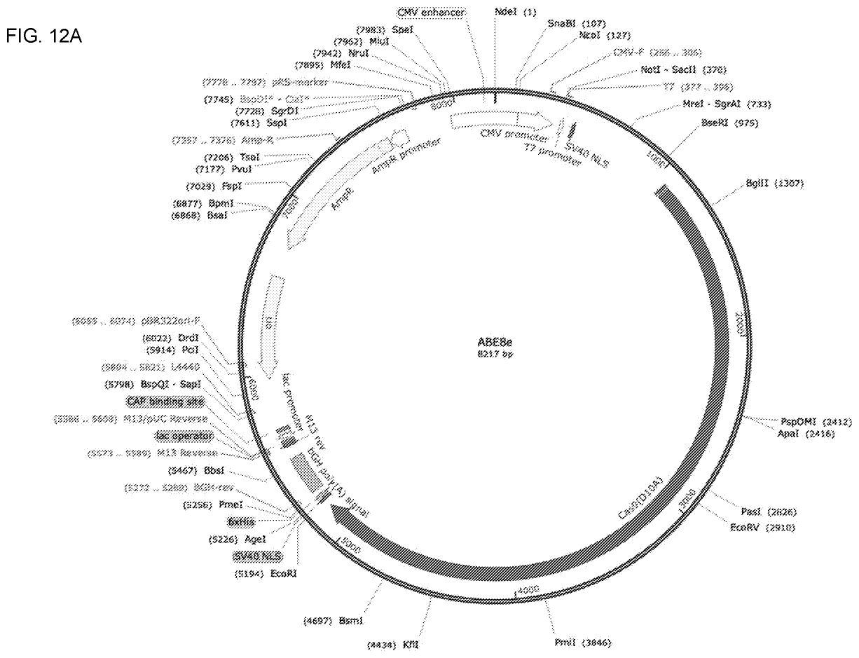

FIGS. 9A-9B illustrate correlation between in vivo CD33 editing levels and protection from GO-induced cytotoxicity. Three mice per group were treated with GO (FIG. 9A), and a sharp decrease in the number of CD14+ monocytes was observed 1 week post treatment, showing that the drug is active. The magnitude of the decrease was inversely correlated with editing efficiency. The sharper decrease was seen in the control group and a smaller effect was observed in the CRISPR group where editing efficiency was highest. FIG. 9B shows the parallel control experiment, without GO treatment. FIGS. 10A-10B further illustrate recovery of CD33 expression in HSCs following administration of GO. No effect of GO on CD33 negative cell lineages was observed (FIGS. 11A-11B). The recovery in CD14+ cell number over time suggests that progenitor or stem cells are able to replenish the pool of monocytes and were thus not affected by treatment. Data demonstrate that, as compared to base edited HSCs, CRISPR edited HSCs are not well suited to survival in vivo (FIG. 9B).

Discussion

Two approaches were pursued to inactivate CD33. In the first approach, a premature stop codon was introduced with CBE into CD33 exon 2 that is predicted to either produce a premature and non-functional protein, or no protein at all if the messenger RNA is degraded by the non-sense mediated decay pathway (Hug et al., Nucleic Acids Res. 44(4): 1483-1495, 2016). In the second approach, conserved splicing donor or acceptor site were targeted to dysregulate CD33 splicing process and inactivate expression. Using CBE, it was demonstrated in this example that the second approach (targeting a splicing site) was more efficient at inactivating CD33; subsequent experiments using ABE were conducted with this approach.

### Example 4: Characterization of Base Editor Modified Cells in Non-Human Primates

This example provides an exemplary method for autologous transplantation of BE edited cells into non-human primates, and methods for analysis of the resultant biological activity.

Autologous NHP transplantation, priming (mobilization), collection of cells, and genetic engineering are conducted consistent with previously published protocols (Trobridge et al., Blood 111(12):5537-5543, 2008; PMID: 18388180). In parallel to cell processing, macaques are conditioned with myeloablative TBI of 1020 cGy from a 6-MV x-ray beam of a single-source linear accelerator located at the Fred Hutch South Lake Union Facility (Seattle, Wash., USA); irradiation is administered as a fractionated dose over the 2 days before cell infusion. During irradiation, animals are housed in a specially modified cage that provides unrestricted access for the irradiation while simultaneously minimizing excess movement. The dose is administered at a rate of 7 cGy/min delivered as a midline tissue dose. Granulocyte colony-stimulating factor is administered daily from the day of cell infusion until the animals began to show onset of neutrophil recovery. Supportive care, including antibiotics, electrolytes, fluids, and transfusions, is given as necessary, and blood counts are analyzed daily to monitor hematopoietic recovery.

NHP-primed BM is harvested, enriched, and cultured as previously described (Trobridge et al., Blood 111(12):5537-5543, 2008; PMID: 18388180). Briefly, before enrichment of CD34+ cells, red cells are lysed in ammonium chloride lysis buffer, and white blood cells are incubated for 20 min with the 12.8 immuno- globulin M anti-CD34 antibody and then washed and incubated for another 20 min with magnetic-activated cell-sorting anti-immunoglobulin M microbeads (Miltenyi Biotec). The cell suspension is run through magnetic columns enriching for CD34+ cell fractions with a purity of 60 to

80% confirmed by flow cytometry. Human CD34+ cells are harvested and enriched from mobilized PB as previously described (Adair et al., Nat. Commun. 7:13173, 2016, DOI: 10.1083/ncomms13173). Enriched CD34+ cells are cultured in stemspan serum-free expansion medium II (SFEM II) (STEMCELL Technologies) supplemented with penicillin and streptomycin (100 U/ml; Gibco by Life Technologies), stem cell factor (PeproTech), thrombopoietin (PeproTech), and Fms-related tyrosine kinase 3 ligand (Miltenyi Biotec) (100 ng/ml for each cytokine).

The art recognizes methods for evaluating biological function(s) of modified cells in non-human primates, based for instance on the modifications included in the introduced cells. See, for instance, methods described in Humber et al. (Leukemia 33:762-808, 2019) for evaluating CD33 modifications; and Humbert et al. (Mol. Ther. Meth. & Clin. Dev. 8:75-86, 2018) for evaluating hematopoietic stem cell gene editing for β-hemoglobinopathies.

### Example 5: In Vivo CD33-Inactivation for Selection of Therapeutically Engineered HSCs

This Example illustrates in vivo genetic engineering of HSCs, where inactivation of CD33 provides a means for selection of engineered HSCs by administration of an anti-CD33 agent. In the present Example, genetic engineering of HSCs in vivo is achieved using a viral vector, e.g., a helper-dependent adenoviral vector such as a helper dependent Ad35 viral vector.

In a first therapy component, a subject can receive an immunosuppressive conditioning regimen to reduce or control the immune reaction to administration of the viral vector. As one example of a conditioning regimen, a subject can be administered tacrolimus, dexamethasone, anakinra, and/or tocilizumab. For example, in one particular regimen, a subject is administered (i) tacrolimus for 4 days prior to vector administration, on each day of vector administration, and for two days after the last day of vector administration; (ii) dexamethasone for one day prior to vector administration, and on each day of vector administration; (iii) anakinra on each day of vector administration; and (iv) tocilizumab on each day of vector administration.

In a second therapy component, HSCs of a human subject are mobilized by administration to the subject of a mobilization regimen. As one example of HSC mobilization, a subject can be administered G-CSF and AMD3100 prior to administration of a viral vector. A particular mobilization regimen can include (i) administration of G-CSF on each of 4 days prior to vector administration and on the first day of vector administration; and (ii) administration of AMD3100 one day prior to vector administration and on the first day of vector administration. Other mobilizing agents are known in the art and disclosed herein. For example, a mobilizing agent can be or include a Gro-Beta agent, e.g., as disclosed in WO 2019/089833 (e.g., Gro-Beta, Gro-BetaT, and a variant thereof), WO 2019/113375, and/or WO 2019/136159, each of which is incorporated herein by reference in its entirety and in particular with respect to sequences relating to Gro-Beta and modified forms thereof. One exemplary Gro-Beta agent is MGTA 145 (Magenta Therapeutics). Certain Gro-Beta agents do not include amino acids corresponding to the four N-terminal amino acids of canonical Gro-Beta. In a third therapy component, the subject can be administered a viral vector, e.g., a viral vector that selectively transduces HSCs (e.g., a helper-dependent adenoviral vector that selectively transduces HSCs such as a helper dependent Ad35 viral vector), by injection in a single dose or in two doses administered on consecutive days A viral vector can encode a base editing system that includes an ABE or CBE and an sgRNA for inactivation of CD33. A viral vector can further encode a therapeutic payload for integration of a therapeutic transgene into the genome of a target cell and/or one or more additional sgRNAs, e.g., for treatment of a condition unrelated to CD33. For example, the condition unrelated to CD33 could be a hemoglobinopathy and the therapeutic payload and/or additional sgRNA(s) could include a transgene or sgRNA engineered cause an increase in gamma globin expression. Related application No. PCT/US2020/040756 is incorporated herein by reference in its entirety and with respect to adenoviral vectors, in particular with respect to Ad35 vectors, including HDAd35 vectors and related vectors.

In a fourth therapy component, subsequent to administration of a final dose of viral vector, a subject can be administered an anti-CD33 agent that eliminates cells (e.g., eliminates HSCs) in which CD33 is not inactivated. In various examples, an anti-CD33 agent can be administered at one or more times selected from any of one or more days on which vector is administered or a date that is at least one day after the day on which the final dose of vector is administered, e.g., 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 6 months, 9 months, or 1 year after the day on which the final dose of vector is administered. Administration of an anti-CD33 agent will selectively increase the frequency of engineered HSCs relative to other HSCs as compared to a reference to which the anti-CD33 agent is not administered. As those of skill in the art will appreciate, increasing and/or maintaining the population of therapeutically engineered cells in the subject's HSC population by selection for CD33-inactivated HSCs will increase therapeutic efficacy.

### Closing Paragraphs.

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means includes, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. A material effect would cause a statistically-significant reduction in resistance to a CD33 targeting therapy in cells genetically modified with a base editing system to reduce CD33 as disclosed herein.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching(s). In addition to the foregoing, base-editing systems described in the following publications are also expressly incorporated by reference herein: Gerkhe et al., Nat. Biotechnol., 10.1038/nbt.4199 (2018); Hu et al., Nature, 556, 57-63 (2018); Jiang et al., Cell. Res, 10.1038/s41422-018-0052-4 (2018); Kim et al., Nat. Biotechnol. 35, 475-480 (2017); Koblan et al., Nat. Biotechnol 36(9):843-846 (2018); Komer et al., Nature, 533, 420-424, (2016); Komer et al., Sci. Adv., 3, eaao4774, (2017); Li et al., Nat. Biotechnol. 36, 324-327 (2018); Nishida et al., Science, 353(6305), 10.1126/science.aaf8729 (2016); Nishimasu et al., Science; 361(6408): 1259-1262 (2018); Rees et al., Nat. Commun. 8, 15790, (2017); Rees & Liu Nat. Rev Genet. 19(12): 770-788 (2018); and Wang et al., Nat. Biotechnol. 10.1038/nbt.4198 (2018).

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the following examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Ed. Anthony Smith, Oxford University Press, Oxford, 2004).

### REFERENCE TO SEQUENCE LISTING

The nucleic acid and/or amino acid sequences described herein are shown using standard letter abbreviations, as defined in 37 C.F.R. § 1.822. Only one strand of each nucleic acid sequence is shown, but the complementary strand is understood as included in embodiments where it would be appropriate. A computer readable text file, entitled “20N4892.txt (Sequence Listing.txt)” created on or about Apr. 21, 2022, with a file size of 836 KB, contains the sequence listing for this application and is hereby incorporated by reference in its entirety.

The following sequences are included in this application:

MfCD33_G7PYH0
MPLLLLPLLWAGALAMDPRVRLEVQESVTVOEGLCVLVPCTFFHPVPYHTRNSPVHGYWFREGAIVSLDSPVAT
NKLDQEVQEETQGRFRLLGDPSRNNCSLSIVDARRRDNGSYFFRMEKGSTKYSYKSTQLSVHVTDLTHRPQILI
PGALDPDHSKNLTCSVPWACEQGTPPIFSWMSAAPTSLGLRTTHSSVLIITPRPQDHGTNLTCQVKFPGAGVTT
ERTIQLNVSYASQNPRTDIFLGDGSGRKARKQGVVQGAIGGAGVTVLLALCLCLIFFTVQ (SEQ ID NO:
1)
HsCD33_P20138
MPLLLLLPLLWAGALAMDPNFWLQVQESVTVQEGLCVLVPCTFFHPIPYYDKNSPVHGYWFREGAIISRDSPVA
TNKLDQEVQEETQGRFRLLGDPSRNNCSLSIVDARRRDNGSYFFRMERGSTKYSYKSPQLSVHVTDLTHRPKIL
IPGTLEPGHSKNLTCSVSWACEQGTPPIFSWLSAAPTSLGPRTTHSSVLIITPRPQDHGTNLTCQVKFAGAGVT
TERTIQLNVTYVPQNPTTGIFPGDGSGKQETRAGVVHGAIGGAGVTALLALCLCLIFFIVQ (SEQ ID NO:
2)
MmCD33_Q63994
MLWPLPLFLLCAGSLAQDLEFQLVAPESVTVEEGLCVHVPCSVFYPSIKLTLGPVTGSWLRKGVSLHEDSPVAT
SDPRQLVQKATQGRFQLLGDPQKHDCSLFIRDAQKNDTGMYFFRVVREPFVRYSYKKSQLSLHVTSLSRTPDII
IPGTLEAGYPSNLTCSVPWACEQGTPPTFSWMSTALTSLSSRTTDSSVLTFTPQPQDHGTKLTCLVTFSGAGVT
VERTIQLNVTRKSGQMRELVLVAVGEATVKLLILGLCLVFLIVME (SEQ ID NO: 3)
Inactivate intron 1 splicing donor site:
CCCCTGCTGTGGGCAGGTGAGTG (SEQ ID NO: 4)
Introduce stop codon in exon2:
CCCCAGTTCATGGTTACTGGTTC (SEQ ID NO: 5)
ABE8e (Addgene #138489)
ATATGCCAAGTACGCCCCCTATTGACGTCAATGACGGTAAATGGCCCGCCTGGCATTATGCCCAGTACATGACC
TTATGGGACTTTCCTACTTGGCAGTACATCTACGTATTAGTCATCGCTATTACCATGGTGATGCGGTTTTGGCA
GTACATCAATGGGCGTGGATAGCGGTTTGACTCACGGGGATTTCCAAGTCTCCACCCCATTGACGTCAATGGGA
GTTTGTTTTGGCACCAAAATCAACGGGACTTTCCAAAATGTCGTAACAACTCCGCCCCATTGACGCAAATGGGC
GGTAGGCGTGTACGGTGGGAGGTCTATATAAGCAGAGCTGGTTTAGTGAACCGTCAGATCCGCTAGAGATCCGC
GGCCGCTAATACGACTCACTATAGGGAGAGCCGCCACCATGAAACGGACAGCCGACGGAAGCGAGTTCGAGTCA
CCAAAGAAGAAGCGGAAAGTCTCTGAGGTGGAGTTTTCCCACGAGTACTGGATGAGACATGCCCTGACCCTGGC
CAAGAGGGCACGGGATGAGAGGGAGGTGCCTGTGGGAGCCGTGCTGGTGCTGAACAATAGAGTGATCGGCGAGG
GCTGGAACAGAGCCATCGGCCTGCACGACCCAACAGCCCATGCCGAAATTATGGCCCTGAGACAGGGCGGCCTG
GTCATGCAGAACTACAGACTGATTGACGCCACCCTGTACGTGACATTCGAGCCTTGCGTGATGTGCGCCGGCGC
CATGATCCACTCTAGGATCGGCCGCGTGGTGTTTGGCGTGAGGAACTCAAAAAGAGGCGCCGCAGGCTCCCTGA
TGAACGTGCTGAACTACCCCGGCATGAATCACCGCGTCGAAATTACCGAGGGAATCCTGGCAGATGAATGTGCC
GCCCTGCTGTGCGATTTCTATCGGATGCCTAGACAGGTGTTCAATGCTCAGAAGAAGGCCCAGAGCTCCATCAA
CTCCGGAGGATCTAGCGGAGGCTCCTCTGGCTCTGAGACACCTGGCACAAGCGAGAGCGCAACACCTGAAAGCA
GCGGGGGCAGCAGCGGGGGGTCAGACAAGAAGTACAGCATCGGCCTGGCCATCGGCACCAACTCTGTGGGCTGG
GCCGTGATCACCGACGAGTACAAGGTGCCCAGCAAGAAATTCAAGGTGCTGGGCAACACCGACCGGCACAGCAT
CAAGAAGAACCTGATCGGAGCCCTGCTGTTCGACAGCGGCGAAACAGCCGAGGCCACCCGGCTGAAGAGAACCG
CCAGAAGAAGATACACCAGACGGAAGAACCGGATCTGCTATCTGCAAGAGATCTTCAGCAACGAGATGGCCAAG
GTGGACGACAGCTTCTTCCACAGACTGGAAGAGTCCTTCCTGGTGGAAGAGGATAAGAAGCACGAGCGGCACCC
CATCTTCGGCAACATCGTGGACGAGGTGGCCTACCACGAGAAGTACCCCACCATCTACCACCTGAGAAAGAAAC
TGGTGGACAGCACCGACAAGGCCGACCTGCGGCTGATCTATCTGGCCCTGGCCCACATGATCAAGTTCCGGGGC
CACTTCCTGATCGAGGGCGACCTGAACCCCGACAACAGCGACGTGGACAAGCTGTTCATCCAGCTGGTGCAGAC
CTACAACCAGCTGTTCGAGGAAAACCCCATCAACGCCAGCGGCGTGGACGCCAAGGCCATCCTGTCTGCCAGAC
TGAGCAAGAGCAGACGGCTGGAAAATCTGATCGCCCAGCTGCCCGGCGAGAAGAAGAATGGCCTGTTCGGAAAC
CTGATTGCCCTGAGCCTGGGCCTGACCCCCAACTTCAAGAGCAACTTCGACCTGGCCGAGGATGCCAAACTGCA
GCTGAGCAAGGACACCTACGACGACGACCTGGACAACCTGCTGGCCCAGATCGGCGACCAGTACGCCGACCTGT
TTCTGGCCGCCAAGAACCTGTCCGACGCCATCCTGCTGAGCGACATCCTGAGAGTGAACACCGAGATCACCAAG
GCCCCCCTGAGCGCCTCTATGATCAAGAGATACGACGAGCACCACCAGGACCTGACCCTGCTGAAAGCTCTCGT
GCGGCAGCAGCTGCCTGAGAAGTACAAAGAGATTTTCTTCGACCAGAGCAAGAACGGCTACGCCGGCTACATTG
ACGGCGGAGCCAGCCAGGAAGAGTTCTACAAGTTCATCAAGCCCATCCTGGAAAAGATGGACGGCACCGAGGAA
CTGCTCGTGAAGCTGAACAGAGAGGACCTGCTGCGGAAGCAGCGGACCTTCGACAACGGCAGCATCCCCCACCA
GATCCACCTGGGAGAGCTGCACGCCATTCTGCGGCGGCAGGAAGATTTTTACCCATTCCTGAAGGACAACCGGG
AAAAGATCGAGAAGATCCTGACCTTCCGCATCCCCTACTACGTGGGCCCTCTGGCCAGGGGAAACAGCAGATTC
GCCTGGATGACCAGAAAGAGCGAGGAAACCATCACCCCCTGGAACTTCGAGGAAGTGGTGGACAAGGGCGCTTC
CGCCCAGAGCTTCATCGAGCGGATGACCAACTTCGATAAGAACCTGCCCAACGAGAAGGTGCTGCCCAAGCACA
GCCTGCTGTACGAGTACTTCACCGTGTATAACGAGCTGACCAAAGTGAAATACGTGACCGAGGGAATGAGAAAG
CCCGCCTTCCTGAGCGGCGAGCAGAAAAAGGCCATCGTGGACCTGCTGTTCAAGACCAACCGGAAAGTGACCGT
GAAGCAGCTGAAAGAGGACTACTTCAAGAAAATCGAGTGCTTCGACTCCGTGGAAATCTCCGGCGTGGAAGATC
GGTTCAACGCCTCCCTGGGCACATACCACGATCTGCTGAAAATTATCAAGGACAAGGACTTCCTGGACAATGAG
GAAAACGAGGACATTCTGGAAGATATCGTGCTGACCCTGACACTGTTTGAGGACAGAGAGATGATCGAGGAACG
GCTGAAAACCTATGCCCACCTGTTCGACGACAAAGTGATGAAGCAGCTGAAGCGGCGGAGATACACCGGCTGGG
GCAGGCTGAGCCGGAAGCTGATCAACGGCATCCGGGACAAGCAGTCCGGCAAGACAATCCTGGATTTCCTGAAG
TCCGACGGCTTCGCCAACAGAAACTTCATGCAGCTGATCCACGACGACAGCCTGACCTTTAAAGAGGACATCCA
GAAAGCCCAGGTGTCCGGCCAGGGCGATAGCCTGCACGAGCACATTGCCAATCTGGCCGGCAGCCCCGCCATTA
AGAAGGGCATCCTGCAGACAGTGAAGGTGGTGGACGAGCTCGTGAAAGTGATGGGCCGGCACAAGCCCGAGAAC
ATCGTGATCGAAATGGCCAGAGAGAACCAGACCACCCAGAAGGGACAGAAGAACAGCCGCGAGAGAATGAAGCG
GATCGAAGAGGGCATCAAAGAGCTGGGCAGCCAGATCCTGAAAGAACACCCCGTGGAAAACACCCAGCTGCAGA
ACGAGAAGCTGTACCTGTACTACCTGCAGAATGGGCGGGATATGTACGTGGACCAGGAACTGGACATCAACCGG
CTGTCCGACTACGATGTGGACCATATCGTGCCTCAGAGCTTTCTGAAGGACGACTCCATCGACAACAAGGTGCT
GACCAGAAGCGACAAGAACCGGGGCAAGAGCGACAACGTGCCCTCCGAAGAGGTCGTGAAGAAGATGAAGAACT
ACTGGCGGCAGCTGCTGAACGCCAAGCTGATTACCCAGAGAAAGTTCGACAATCTGACCAAGGCCGAGAGAGGC
GGCCTGAGCGAACTGGATAAGGCCGGCTTCATCAAGAGACAGCTGGTGGAAACCCGGCAGATCACAAAGCACGT
GGCACAGATCCTGGACTCCCGGATGAACACTAAGTACGACGAGAATGACAAGCTGATCCGGGAAGTGAAAGTGA
TCACCCTGAAGTCCAAGCTGGTGTCCGATTTCCGGAAGGATTTCCAGTTTTACAAAGTGCGCGAGATCAACAAC
TACCACCACGCCCACGACGCCTACCTGAACGCCGTCGTGGGAACCGCCCTGATCAAAAAGTACCCTAAGCTGGA
AAGCGAGTTCGTGTACGGCGACTACAAGGTGTACGACGTGCGGAAGATGATCGCCAAGAGCGAGCAGGAAATCG
GCAAGGCTACCGCCAAGTACTTCTTCTACAGCAACATCATGAACTTTTTCAAGACCGAGATTACCCTGGCCAAC
GGCGAGATCCGGAAGCGGCCTCTGATCGAGACAAACGGCGAAACCGGGGAGATCGTGTGGGATAAGGGCCGGGA
TTTTGCCACCGTGCGGAAAGTGCTGAGCATGCCCCAAGTGAATATCGTGAAAAAGACCGAGGTGCAGACAGGCG
GCTTCAGCAAAGAGTCTATCCTGCCCAAGAGGAACAGCGATAAGCTGATCGCCAGAAAGAAGGACTGGGACCCT
AAGAAGTACGGCGGCTTCGACAGCCCCACCGTGGCCTATTCTGTGCTGGTGGTGGCCAAAGTGGAAAAGGGCAA
GTCCAAGAAACTGAAGAGTGTGAAAGAGCTGCTGGGGATCACCATCATGGAAAGAAGCAGCTTCGAGAAGAATC
CCATCGACTTTCTGGAAGCCAAGGGCTACAAAGAAGTGAAAAAGGACCTGATCATCAAGCTGCCTAAGTACTCC
CTGTTCGAGCTGGAAAACGGCCGGAAGAGAATGCTGGCCTCTGCCGGCGAACTGCAGAAGGGAAACGAACTGGC
CCTGCCCTCCAAATATGTGAACTTCCTGTACCTGGCCAGCCACTATGAGAAGCTGAAGGGCTCCCCCGAGGATA
ATGAGCAGAAACAGCTGTTTGTGGAACAGCACAAGCACTACCTGGACGAGATCATCGAGCAGATCAGCGAGTTC
TCCAAGAGAGTGATCCTGGCCGACGCTAATCTGGACAAAGTGCTGTCCGCCTACAACAAGCACCGGGATAAGCC
CATCAGAGAGCAGGCCGAGAATATCATCCACCTGTTTACCCTGACCAATCTGGGAGCCCCTGCCGCCTTCAAGT
ACTTTGACACCACCATCGACCGGAAGAGGTACACCAGCACCAAAGAGGTGCTGGACGCCACCCTGATCCACCAG
AGCATCACCGGCCTGTACGAGACACGGATCGACCTGTCTCAGCTGGGAGGTGACTCTGGCGGCTCAAAAAGAAC
CGCCGACGGCAGCGAATTCGAGCCCAAGAAGAAGAGGAAAGTCTAACCGGTCATCATCACCATCACCATTGAGT
TTAAACCCGCTGATCAGCCTCGACTGTGCCTTCTAGTTGCCAGCCATCTGTTGTTTGCCCCTCCCCCGTGCCTT
CCTTGACCCTGGAAGGTGCCACTCCCACTGTCCTTTCCTAATAAAATGAGGAAATTGCATCGCATTGTCTGAGT
AGGTGTCATTCTATTCTGGGGGGTGGGGTGGGGCAGGACAGCAAGGGGGAGGATTGGGAAGACAATAGCAGGCA
TGCTGGGGATGCGGTGGGCTCTATGGCTTCTGAGGCGGAAAGAACCAGCTGGGGCTCGATACCGTCGACCTCTA
GCTAGAGCTTGGCGTAATCATGGTCATAGCTGTTTCCTGTGTGAAATTGTTATCCGCTCACAATTCCACACAAC
ATACGAGCCGGAAGCATAAAGTGTAAAGCCTAGGGTGCCTAATGAGTGAGCTAACTCACATTAATTGCGTTGCG
CTCACTGCCCGCTTTCCAGTCGGGAAACCTGTCGTGCCAGCTGCATTAATGAATCGGCCAACGCGCGGGGAGAG
GCGGTTTGCGTATTGGGCGCTCTTCCGCTTCCTCGCTCACTGACTCGCTGCGCTCGGTCGTTCGGCTGCGGCGA
GCGGTATCAGCTCACTCAAAGGCGGTAATACGGTTATCCACAGAATCAGGGGATAACGCAGGAAAGAACATGTG
AGCAAAAGGCCAGCAAAAGGCCAGGAACCGTAAAAAGGCCGCGTTGCTGGCGTTTTTCCATAGGCTCCGCCCCC
CTGACGAGCATCACAAAAATCGACGCTCAAGTCAGAGGTGGCGAAACCCGACAGGACTATAAAGATACCAGGCG
TTTCCCCCTGGAAGCTCCCTCGTGCGCTCTCCTGTTCCGACCCTGCCGCTTACCGGATACCTGTCCGCCTTTCT
CCCTTCGGGAAGCGTGGCGCTTTCTCATAGCTCACGCTGTAGGTATCTCAGTTCGGTGTAGGTCGTTCGCTCCA
AGCTGGGCTGTGTGCACGAACCCCCCGTTCAGCCCGACCGCTGCGCCTTATCCGGTAACTATCGTCTTGAGTCC
AACCCGGTAAGACACGACTTATCGCCACTGGCAGCAGCCACTGGTAACAGGATTAGCAGAGCGAGGTATGTAGG
CGGTGCTACAGAGTTCTTGAAGTGGTGGCCTAACTACGGCTACACTAGAAGAACAGTATTTGGTATCTGCGCTC
TGCTGAAGCCAGTTACCTTCGGAAAAAGAGTTGGTAGCTCTTGATCCGGCAAACAAACCACCGCTGGTAGCGGT
GGTTTTTTTGTTTGCAAGCAGCAGATTACGCGCAGAAAAAAAGGATCTCAAGAAGATCCTTTGATCTTTTCTAC
GGGGTCTGACACTCAGTGGAACGAAAACTCACGTTAAGGGATTTTGGTCATGAGATTATCAAAAAGGATCTTCA
CCTAGATCCTTTTAAATTAAAAATGAAGTTTTAAATCAATCTAAAGTATATATGAGTAAACTTGGTCTGACAGT
TACCAATGCTTAATCAGTGAGGCACCTATCTCAGCGATCTGTCTATTTCGTTCATCCATAGTTGCCTGACTCCC
CGTCGTGTAGATAACTACGATACGGGAGGGCTTACCATCTGGCCCCAGTGCTGCAATGATACCGCGAGACCCAC
GCTCACCGGCTCCAGATTTATCAGCAATAAACCAGCCAGCCGGAAGGGCCGAGCGCAGAAGTGGTCCTGCAACT
TTATCCGCCTCCATCCAGTCTATTAATTGTTGCCGGGAAGCTAGAGTAAGTAGTTCGCCAGTTAATAGTTTGCG
CAACGTTGTTGCCATTGCTACAGGCATCGTGGTGTCACGCTCGTCGTTTGGTATGGCTTCATTCAGCTCCGGTT
CCCAACGATCAAGGCGAGTTACATGATCCCCCATGTTGTGCAAAAAAGCGGTTAGCTCCTTCGGTCCTCCGATC
GTTGTCAGAAGTAAGTTGGCCGCAGTGTTATCACTCATGGTTATGGCAGCACTGCATAATTCTCTTACTGTCAT
GCCATCCGTAAGATGCTTTTCTGTGACTGGTGAGTACTCAACCAAGTCATTCTGAGAATAGTGTATGCGGCGAC
CGAGTTGCTCTTGCCCGGCGTCAATACGGGATAATACCGCGCCACATAGCAGAACTTTAAAAGTGCTCATCATT
GGAAAACGTTCTTCGGGGCGAAAACTCTCAAGGATCTTACCGCTGTTGAGATCCAGTTCGATGTAACCCACTCG
TGCACCCAACTGATCTTCAGCATCTTTTACTTTCACCAGCGTTTCTGGGTGAGCAAAAACAGGAAGGCAAAATG
CCGCAAAAAAGGGAATAAGGGCGACACGGAAATGTTGAATACTCATACTCTTCCTTTTTCAATATTATTGAAGC
ATTTATCAGGGTTATTGTCTCATGAGCGGATACATATTTGAATGTATTTAGAAAAATAAACAAATAGGGGTTCC
GCGCACATTTCCCCGAAAAGTGCCACCTGACGTCGACGGATCGGGAGATCGATCTCCCGATCCCCTAGGGTCGA
CTCTCAGTACAATCTGCTCTGATGCCGCATAGTTAAGCCAGTATCTGCTCCCTGCTTGTGTGTTGGAGGTCGCT
GAGTAGTGCGCGAGCAAAATTTAAGCTACAACAAGGCAAGGCTTGACCGACAATTGCATGAAGAATCTGCTTAG
GGTTAGGCGTTTTGCGCTGCTTCGCGATGTACGGGCCAGATATACGCGTTGACATTGATTATTGACTAGTTATT
AATAGTAATCAATTACGGGGTCATTAGTTCATAGCCCATATATTGAGTTCCGCGTTACATAACTTACGGTAAAT
GGCCCGCCTGGCTGACCGCCCAACGACCCCCGCCCATTGACGTCAATAATGACGTATGTTCCCATAGTAACGCC
AATAGGGACTTTCCATTGACGTCAATGGGTGGAGTATTTACGGTAAACTGCCCACTTGGCAGTACATCAAGTGT
ATC (SEQ ID NO: 6)
ABE8e-NG (Addgene #138491) (Based on Next Gen sequencing)
ATATGCCAAGTACGCCCCCTATTGACGTCAATGACGGTAAATGGCCCGCCTGGCATTATGCCCAGTACATGACC
TTATGGGACTTTCCTACTTGGCAGTACATCTACGTATTAGTCATCGCTATTACCATGGTGATGCGGTTTTGGCA
GTACATCAATGGGCGTGGATAGCGGTTTGACTCACGGGGATTTCCAAGTCTCCACCCCATTGACGTCAATGGGA
GTTTGTTTTGGCACCAAAATCAACGGGACTTTCCAAAATGTCGTAACAACTCCGCCCCATTGACGCAAATGGGC
GGTAGGCGTGTACGGTGGGAGGTCTATATAAGCAGAGCTGGTTTAGTGAACCGTCAGATCCGCTAGAGATCCGC
GGCCGCTAATACGACTCACTATAGGGAGAGCCGCCACCATGAAACGGACAGCCGACGGAAGCGAGTTCGAGTCA
CCAAAGAAGAAGCGGAAAGTCTCTGAGGTGGAGTTTTCCCACGAGTACTGGATGAGACATGCCCTGACCCTGGC
CAAGAGGGCACGGGATGAGAGGGAGGTGCCTGTGGGAGCCGTGCTGGTGCTGAACAATAGAGTGATCGGCGAGG
GCTGGAACAGAGCCATCGGCCTGCACGACCCAACAGCCCATGCCGAAATTATGGCCCTGAGACAGGGCGGCCTG
GTCATGCAGAACTACAGACTGATTGACGCCACCCTGTACGTGACATTCGAGCCTTGCGTGATGTGCGCCGGCGC
CATGATCCACTCTAGGATCGGCCGCGTGGTGTTTGGCGTGAGGAACTCAAAAAGAGGCGCCGCAGGCTCCCTGA
TGAACGTGCTGAACTACCCCGGCATGAATCACCGCGTCGAAATTACCGAGGGAATCCTGGCAGATGAATGTGCC
GCCCTGCTGTGCGATTTCTATCGGATGCCTAGACAGGTGTTCAATGCTCAGAAGAAGGCCCAGAGCTCCATCAA
CTCCGGAGGATCTAGCGGAGGCTCCTCTGGCTCTGAGACACCTGGCACAAGCGAGAGCGCAACACCTGAAAGCA
GCGGGGGCAGCAGCGGGGGGTCAGACAAGAAGTACAGCATCGGCCTGGCCATCGGCACCAACTCTGTGGGCTGG
GCCGTGATCACCGACGAGTACAAGGTGCCCAGCAAGAAATTCAAGGTGCTGGGCAACACCGACCGGCACAGCAT
CAAGAAGAACCTGATCGGAGCCCTGCTGTTCGACAGCGGCGAAACAGCCGAGGCCACCCGGCTGAAGAGAACCG
CCAGAAGAAGATACACCAGACGGAAGAACCGGATCTGCTATCTGCAAGAGATCTTCAGCAACGAGATGGCCAAG
GTGGACGACAGCTTCTTCCACAGACTGGAAGAGTCCTTCCTGGTGGAAGAGGATAAGAAGCACGAGCGGCACCC
CATCTTCGGCAACATCGTGGACGAGGTGGCCTACCACGAGAAGTACCCCACCATCTACCACCTGAGAAAGAAAC
TGGTGGACAGCACCGACAAGGCCGACCTGCGGCTGATCTATCTGGCCCTGGCCCACATGATCAAGTTCCGGGGC
CACTTCCTGATCGAGGGCGACCTGAACCCCGACAACAGCGACGTGGACAAGCTGTTCATCCAGCTGGTGCAGAC
CTACAACCAGCTGTTCGAGGAAAACCCCATCAACGCCAGCGGCGTGGACGCCAAGGCCATCCTGTCTGCCAGAC
TGAGCAAGAGCAGACGGCTGGAAAATCTGATCGCCCAGCTGCCCGGCGAGAAGAAGAATGGCCTGTTCGGAAAC
CTGATTGCCCTGAGCCTGGGCCTGACCCCCAACTTCAAGAGCAACTTCGACCTGGCCGAGGATGCCAAACTGCA
GCTGAGCAAGGACACCTACGACGACGACCTGGACAACCTGCTGGCCCAGATCGGCGACCAGTACGCCGACCTGT
TTCTGGCCGCCAAGAACCTGTCCGACGCCATCCTGCTGAGCGACATCCTGAGAGTGAACACCGAGATCACCAAG
GCCCCCCTGAGCGCCTCTATGATCAAGAGATACGACGAGCACCACCAGGACCTGACCCTGCTGAAAGCTCTCGT
GCGGCAGCAGCTGCCTGAGAAGTACAAAGAGATTTTCTTCGACCAGAGCAAGAACGGCTACGCCGGCTACATTG
ACGGCGGAGCCAGCCAGGAAGAGTTCTACAAGTTCATCAAGCCCATCCTGGAAAAGATGGACGGCACCGAGGAA
CTGCTCGTGAAGCTGAACAGAGAGGACCTGCTGCGGAAGCAGCGGACCTTCGACAACGGCAGCATCCCCCACCA
GATCCACCTGGGAGAGCTGCACGCCATTCTGCGGCGGCAGGAAGATTTTTACCCATTCCTGAAGGACAACCGGG
AAAAGATCGAGAAGATCCTGACCTTCCGCATCCCCTACTACGTGGGCCCTCTGGCCAGGGGAAACAGCAGATTC
GCCTGGATGACCAGAAAGAGCGAGGAAACCATCACCCCCTGGAACTTCGAGGAAGTGGTGGACAAGGGCGCTTC
CGCCCAGAGCTTCATCGAGCGGATGACCAACTTCGATAAGAACCTGCCCAACGAGAAGGTGCTGCCCAAGCACA
GCCTGCTGTACGAGTACTTCACCGTGTATAACGAGCTGACCAAAGTGAAATACGTGACCGAGGGAATGAGAAAG
CCCGCCTTCCTGAGCGGCGAGCAGAAAAAGGCCATCGTGGACCTGCTGTTCAAGACCAACCGGAAAGTGACCGT
GAAGCAGCTGAAAGAGGACTACTTCAAGAAAATCGAGTGCTTCGACTCCGTGGAAATCTCCGGCGTGGAAGATC
GGTTCAACGCCTCCCTGGGCACATACCACGATCTGCTGAAAATTATCAAGGACAAGGACTTCCTGGACAATGAG
GAAAACGAGGACATTCTGGAAGATATCGTGCTGACCCTGACACTGTTTGAGGACAGAGAGATGATCGAGGAACG
GCTGAAAACCTATGCCCACCTGTTCGACGACAAAGTGATGAAGCAGCTGAAGCGGCGGAGATACACCGGCTGGG
GCAGGCTGAGCCGGAAGCTGATCAACGGCATCCGGGACAAGCAGTCCGGCAAGACAATCCTGGATTTCCTGAAG
TCCGACGGCTTCGCCAACAGAAACTTCATGCAGCTGATCCACGACGACAGCCTGACCTTTAAAGAGGACATCCA
GAAAGCCCAGGTGTCCGGCCAGGGCGATAGCCTGCACGAGCACATTGCCAATCTGGCCGGCAGCCCCGCCATTA
AGAAGGGCATCCTGCAGACAGTGAAGGTGGTGGACGAGCTCGTGAAAGTGATGGGCCGGCACAAGCCCGAGAAC
ATCGTGATCGAAATGGCCAGAGAGAACCAGACCACCCAGAAGGGACAGAAGAACAGCCGCGAGAGAATGAAGCG
GATCGAAGAGGGCATCAAAGAGCTGGGCAGCCAGATCCTGAAAGAACACCCCGTGGAAAACACCCAGCTGCAGA
ACGAGAAGCTGTACCTGTACTACCTGCAGAATGGGCGGGATATGTACGTGGACCAGGAACTGGACATCAACCGG
CTGTCCGACTACGATGTGGACCATATCGTGCCTCAGAGCTTTCTGAAGGACGACTCCATCGACAACAAGGTGCT
GACCAGAAGCGACAAGAACCGGGGCAAGAGCGACAACGTGCCCTCCGAAGAGGTCGTGAAGAAGATGAAGAACT
ACTGGCGGCAGCTGCTGAACGCCAAGCTGATTACCCAGAGAAAGTTCGACAATCTGACCAAGGCCGAGAGAGGC
GGCCTGAGCGAACTGGATAAGGCCGGCTTCATCAAGAGACAGCTGGTGGAAACCCGGCAGATCACAAAGCACGT
GGCACAGATCCTGGACTCCCGGATGAACACTAAGTACGACGAGAATGACAAGCTGATCCGGGAAGTGAAAGTGA
TCACCCTGAAGTCCAAGCTGGTGTCCGATTTCCGGAAGGATTTCCAGTTTTACAAAGTGCGCGAGATCAACAAC
TACCACCACGCCCACGACGCCTACCTGAACGCCGTCGTGGGAACCGCCCTGATCAAAAAGTACCCTAAGCTGGA
AAGCGAGTTCGTGTACGGCGACTACAAGGTGTACGACGTGCGGAAGATGATCGCCAAGAGCGAGCAGGAAATCG
GCAAGGCTACCGCCAAGTACTTCTTCTACAGCAACATCATGAACTTTTTCAAGACCGAGATTACCCTGGCCAAC
GGCGAGATCCGGAAGCGGCCTCTGATCGAGACAAACGGCGAAACCGGGGAGATCGTGTGGGATAAGGGCCGGGA
TTTTGCCACCGTGCGGAAAGTGCTGAGCATGCCCCAAGTGAATATCGTGAAAAAGACCGAGGTGCAGACAGGCG
GCTTCAGCAAAGAGTCTATCAGGCCCAAGAGGAACAGCGATAAGCTGATCGCCAGAAAGAAGGACTGGGACCCT
AAGAAGTACGGCGGCTTCGTCAGCCCCACCGTGGCCTATTCTGTGCTGGTGGTGGCCAAAGTGGAAAAGGGCAA
GTCCAAGAAACTGAAGAGTGTGAAAGAGCTGCTGGGGATCACCATCATGGAAAGAAGCAGCTTCGAGAAGAATC
CCATCGACTTTCTGGAAGCCAAGGGCTACAAAGAAGTGAAAAAGGACCTGATCATCAAGCTGCCTAAGTACTCC
CTGTTCGAGCTGGAAAACGGCCGGAAGAGAATGCTGGCCTCTGCCAGATTCCTGCAGAAGGGAAACGAACTGGC
CCTGCCCTCCAAATATGTGAACTTCCTGTACCTGGCCAGCCACTATGAGAAGCTGAAGGGCTCCCCCGAGGATA
ATGAGCAGAAACAGCTGTTTGTGGAACAGCACAAGCACTACCTGGACGAGATCATCGAGCAGATCAGCGAGTTC
TCCAAGAGAGTGATCCTGGCCGACGCTAATCTGGACAAAGTGCTGTCCGCCTACAACAAGCACCGGGATAAGCC
CATCAGAGAGCAGGCCGAGAATATCATCCACCTGTTTACCCTGACCAATCTGGGAGCCCCTAGGGCCTTCAAGT
ACTTTGACACCACCATCGACCGGAAGGTGTACAGGAGCACCAAAGAGGTGCTGGACGCCACCCTGATCCACCAG
AGCATCACCGGCCTGTACGAGACACGGATCGACCTGTCTCAGCTGGGAGGTGACTCTGGCGGCTCAAAAAGAAC
CGCCGACGGCAGCGAATTCGAGCCCAAGAAGAAGAGGAAAGTCTAACCGGTCATCATCACCATCACCATTGAGT
TTAAACCCGCTGATCAGCCTCGACTGTGCCTTCTAGTTGCCAGCCATCTGTTGTTTGCCCCTCCCCCGTGCCTT
CCTTGACCCTGGAAGGTGCCACTCCCACTGTCCTTTCCTAATAAAATGAGGAAATTGCATCGCATTGTCTGAGT
AGGTGTCATTCTATTCTGGGGGGTGGGGTGGGGCAGGACAGCAAGGGGGAGGATTGGGAAGACAATAGCAGGCA
TGCTGGGGATGCGGTGGGCTCTATGGCTTCTGAGGCGGAAAGAACCAGCTGGGGCTCGATACCGTCGACCTCTA
GCTAGAGCTTGGCGTAATCATGGTCATAGCTGTTTCCTGTGTGAAATTGTTATCCGCTCACAATTCCACACAAC
ATACGAGCCGGAAGCATAAAGTGTAAAGCCTAGGGTGCCTAATGAGTGAGCTAACTCACATTAATTGCGTTGCG
CTCACTGCCCGCTTTCCAGTCGGGAAACCTGTCGTGCCAGCTGCATTAATGAATCGGCCAACGCGCGGGGAGAG
GCGGTTTGCGTATTGGGCGCTCTTCCGCTTCCTCGCTCACTGACTCGCTGCGCTCGGTCGTTCGGCTGCGGCGA
GCGGTATCAGCTCACTCAAAGGCGGTAATACGGTTATCCACAGAATCAGGGGATAACGCAGGAAAGAACATGTG
AGCAAAAGGCCAGCAAAAGGCCAGGAACCGTAAAAAGGCCGCGTTGCTGGCGTTTTTCCATAGGCTCCGCCCCC
CTGACGAGCATCACAAAAATCGACGCTCAAGTCAGAGGTGGCGAAACCCGACAGGACTATAAAGATACCAGGCG
TTTCCCCCTGGAAGCTCCCTCGTGCGCTCTCCTGTTCCGACCCTGCCGCTTACCGGATACCTGTCCGCCTTTCT
CCCTTCGGGAAGCGTGGCGCTTTCTCATAGCTCACGCTGTAGGTATCTCAGTTCGGTGTAGGTCGTTCGCTCCA
AGCTGGGCTGTGTGCACGAACCCCCCGTTCAGCCCGACCGCTGCGCCTTATCCGGTAACTATCGTCTTGAGTCC
AACCCGGTAAGACACGACTTATCGCCACTGGCAGCAGCCACTGGTAACAGGATTAGCAGAGCGAGGTATGTAGG
CGGTGCTACAGAGTTCTTGAAGTGGTGGCCTAACTACGGCTACACTAGAAGAACAGTATTTGGTATCTGCGCTC
TGCTGAAGCCAGTTACCTTCGGAAAAAGAGTTGGTAGCTCTTGATCCGGCAAACAAACCACCGCTGGTAGCGGT
GGTTTTTTTGTTTGCAAGCAGCAGATTACGCGCAGAAAAAAAGGATCTCAAGAAGATCCTTTGATCTTTTCTAC
GGGGTCTGACACTCAGTGGAACGAAAACTCACGTTAAGGGATTTTGGTCATGAGATTATCAAAAAGGATCTTCA
CCTAGATCCTTTTAAATTAAAAATGAAGTTTTAAATCAATCTAAAGTATATATGAGTAAACTTGGTCTGACAGT
TACCAATGCTTAATCAGTGAGGCACCTATCTCAGCGATCTGTCTATTTCGTTCATCCATAGTTGCCTGACTCCC
CGTCGTGTAGATAACTACGATACGGGAGGGCTTACCATCTGGCCCCAGTGCTGCAATGATACCGCGAGACCCAC
GCTCACCGGCTCCAGATTTATCAGCAATAAACCAGCCAGCCGGAAGGGCCGAGCGCAGAAGTGGTCCTGCAACT
TTATCCGCCTCCATCCAGTCTATTAATTGTTGCCGGGAAGCTAGAGTAAGTAGTTCGCCAGTTAATAGTTTGCG
CAACGTTGTTGCCATTGCTACAGGCATCGTGGTGTCACGCTCGTCGTTTGGTATGGCTTCATTCAGCTCCGGTT
CCCAACGATCAAGGCGAGTTACATGATCCCCCATGTTGTGCAAAAAAGCGGTTAGCTCCTTCGGTCCTCCGATC
GTTGTCAGAAGTAAGTTGGCCGCAGTGTTATCACTCATGGTTATGGCAGCACTGCATAATTCTCTTACTGTCAT
GCCATCCGTAAGATGCTTTTCTGTGACTGGTGAGTACTCAACCAAGTCATTCTGAGAATAGTGTATGCGGCGAC
CGAGTTGCTCTTGCCCGGCGTCAATACGGGATAATACCGCGCCACATAGCAGAACTTTAAAAGTGCTCATCATT
GGAAAACGTTCTTCGGGGCGAAAACTCTCAAGGATCTTACCGCTGTTGAGATCCAGTTCGATGTAACCCACTCG
TGCACCCAACTGATCTTCAGCATCTTTTACTTTCACCAGCGTTTCTGGGTGAGCAAAAACAGGAAGGCAAAATG
CCGCAAAAAAGGGAATAAGGGCGACACGGAAATGTTGAATACTCATACTCTTCCTTTTTCAATATTATTGAAGC
ATTTATCAGGGTTATTGTCTCATGAGCGGATACATATTTGAATGTATTTAGAAAAATAAACAAATAGGGGTTCC
GCGCACATTTCCCCGAAAAGTGCCACCTGACGTCGACGGATCGGGAGATCGATCTCCCGATCCCCTAGGGTCGA
CTCTCAGTACAATCTGCTCTGATGCCGCATAGTTAAGCCAGTATCTGCTCCCTGCTTGTGTGTTGGAGGTCGCT
GAGTAGTGCGCGAGCAAAATTTAAGCTACAACAAGGCAAGGCTTGACCGACAATTGCATGAAGAATCTGCTTAG
GGTTAGGCGTTTTGCGCTGCTTCGCGATGTACGGGCCAGATATACGCGTTGACATTGATTATTGACTAGTTATT
AATAGTAATCAATTACGGGGTCATTAGTTCATAGCCCATATATTGAGTTCCGCGTTACATAACTTACGGTAAAT
GGCCCGCCTGGCTGACCGCCCAACGACCCCCGCCCATTGACGTCAATAATGACGTATGTTCCCATAGTAACGCC
AATAGGGACTTTCCATTGACGTCAATGGGTGGAGTATTTACGGTAAACTGCCCACTTGGCAGTACATCAAGTGT
ATC (SEQ ID NO: 7)
HBG ″-113 site″ gRNA
CTTGACCAATAGCCTTGACAAGG (SEQ ID NO: 8)
HBG ″-175 site″ gRNA
CACTATCTCAATGCAAATATCTGTCT (SEQ ID NO: 9)
Sequenced region
CTTGACCAATAGCCTTGACA (SEQ ID NO: 10)
Sequenced region
AGATATTTGCATTGAGATAG (SEQ ID NO: 11)
Sequenced region
CCCCACAGGGGCCCTGGCTATGG (SEQ ID NO: 12)
Sequenced region
CCCCACAGGGGCCCTGGCTA (SEQ ID NO: 13)
Full-length CD33
MPLLLLLPLLWAGALAMDPNFWLQVQESVTVQEGLCVLVPCTFFHPIPYYDKNSPVHGYWFREGAIISRDSPVA
TNKLDQEVQEETQGRFRLLGDPSRNNCSLSIVDARRRDNGSYFFRMERGSTKYSYKSPQLSVHVTDLTHRPKIL
IPGTLEPGHSKNLTCSVSWACEQGTPPIFSWLSAAPTSLGPRTTHSSVLIITPRPQDHGTNLTCQVKFAGAGVT
TERTIQLNVTYVPQNPTTGIFPGDGSGKQETRAGVVHGAIGGAGVTALLALCLCLIFFIVKTHRRKAARTAVGR
NDTHPTTGSASPKHQKKSKLHGPTETSSCSGAAPTVEMDEELHYASLNFHGMNPSKDTSTEYSEVRTQ (SEQ
ID NO: 14)
SEQ ID NO: 15 is an exemplary human genome sequence encoding CD33
(chr19:51225119-51235676) .

| exonic sequences: | intronic sequence: |
|---|---|
| 1-37 | 38-99 |
| 100-480 | 481-684 |
| 685-963 | 964-1190 |
| 1191-1238 | 1239-3585 |
| 3586-3606 | 3607-5912 |
| 5913-5922 | 5923-5929 |
| 5930-5948 | 5949-6109 |
| 6110-6140 | 6141-6680 |
| 6681-6701 | 6702-10038 |
| 10039-10135 | 10136-10476 |
| 10477-10558 | |

natural variant positions in human population:
28, 103, 126, 255. 267, 730-733, 1220, 1804, 2100-2113, 2802, 3551-3555,
3886, 6043, 6485, 6551, 6621, 6744, 7200, 7226, 8010, 8200, 8715, 8748,
8920, 9094, 9618, 10092, 10544, 10547
ATGCCGCTGCTGCTACTGCTGCCCCTGCTGTGGGCAGGTGAGTGGCTGTGGGGAGAGGGGTTGTCGGGCTGGGCCGA
GCTGACCCTCGTTTCCCCACAGGGGCCCTGGCTATGGATCCAAATTTCTGGCTGCAAGTGCAGGAGTCAGTGACGGT
ACAGGAGGGTTTGTGCGTCCTCGTGCCCTGCACTTTCTTCCATCCCATACCCTACTACGACAAGAACTCCCCAGTTC
ATGGTTACTGGTTCCGGGAAGGAGCCATTATATCCAGGGACTCTCCAGTGGCCACAAACAAGCTAGATCAAGAAGTA
CAGGAGGAGACTCAGGGCAGATTCCGCCTCCTTGGGGATCCCAGTAGGAACAACTGCTCCCTGAGCATCGTAGACGC
CAGGAGGAGGGATAATGGTTCATACTTCTTTCGGATGGAGAGAGGAAGTACCAAATACAGTTACAAATCTCCCCAGC
TCTCTGTGCATGTGACAGGTGAGGCACAGGCTTCAGAAGTGGCCGCAAGGGAAGTTCATGGGTACTGCAGGGCAGGG
CTGGGATGGGACCCTGGTACTGGGAGGGGTTTAGGGGTAAAGCCTGTCGTGCTTAGCGGGGGAGCTTGACCAGAGGT
TGATCTTCTCTCAGGCCCTCACCTGGACCCTCCCTCCTGATTCTGCATCCCCTCTTTCTCCTCACTAGACTTGACCC
ACAGGCCCAAAATCCTCATCCCTGGCACTCTAGAACCCGGCCACTCCAAAAACCTGACCTGCTCTGTGTCCTGGGCC
TGTGAGCAGGGAACACCCCCGATCTTCTCCTGGTTGTCAGCTGCCCCCACCTCCCTGGGCCCCAGGACTACTCACTC
CTCGGTGCTCATAATCACCCCACGGCCCCAGGACCACGGCACCAACCTGACCTGTCAGGTGAAGTTCGCTGGAGCTG
GTGTGACTACGGAGAGAACCATCCAGCTCAACGTCACCTGTAAGTGCTGGGCCAGGATGCTGGGGTCCCTGAGGGTG
TAGGGGAGACAGGATGGGCTGGTGCTGGGGACATTTAGTGTCCTGGAGGCCTGGCTGAGTTCGGGAGCCAGAAGGAC
ATGAGCCCTGTCCCTTCTGCATTTCTGTGGTTTCTGGCAGGAGTAAGGGGAAATGCCTACCCTTATCTCATCTCTAC
CCCCAACTGAAGGAAATCCTCTCTTCCTCTCCTAGATGTTCCACAGAACCCAACAACTGGTATCTTTCCAGGAGATG
GCTCAGGTAGGAAGGAGCCTCCCCGCCTGGGGCTGTTACTGACATTGAGTCTGTGTCAGGTTTGGTCAGATCTGGAC
TTTCAGAGTCAAATGTTCAGAGGCAAGGCCTGCAGTTAGACACGGGTAGACATCAGGCACCTTGGAAAAGGATATTT
GGGGATGACTAGCAACTTCCCCCTTGCCCATCCAAATAATGCTCTTTGTCTCCCTCCTGTCTCTGAATGTCTTGGGG
TATTTTATTTTTAATTGATATGTAATAATAGTACATATTTATGGATGGCATAGTGATGTTTCCATACTAATAATGTA
TAGTAATCAGATCAGGGTAATAGCATATCCATCATCTTGAACATTTATTATTTCATTGTTGTTGGGAACATTCAATA
TCCCCTTTCTAGCTATTTGAAGCTATCTATTATTGTTAAGCATAGTCATCCTACAGTGGTATAGAACACCAGAACTT
ATTCTTCCTTTCCAGGTGTAATCTAGTATCCTTTAACAAATCTCTCTCCTTATCATTGTTCCCCTAACCTTCCCAGC
CCTTATTATTCTCTGTTCTACTTTTTACTTCTATGAAATCAACTTCTTGTAGCTTCCACTTATGAGTGAGAACATGT
GGTATTCAACTTTCTGTTCCTAGCTTATTTCATTTAACATAATGTCCTCTAGTTCAATCTATGTTATAGTGAATAAC
AAGATTTCATTATTTTTTATGGCTGAATGATAATCCATTGTGTATATACGCCACATTTCCTTTATTTATTCATCTGT
TGTTGGACACTTAGGTTTATTTCATATCTTCCTATTGTGGATAATGCTGCAATAAACATTGAGGTGCAGACGTTTCT
TCAATATACTGATTTCCTTTCCTTTCTATAAATGCCCAGTAGTGGGGTTGCTGGATCATATGGTAGTTCTATTTGTA
GTTTTTTGAGAAACTTCCATACTCTTCTCCATAGTGGTTATACTAGTTTACATTCTGGTCAAAAGTATATAAGAGTT
CCCTCTTCTCTACATCCTCACCATCATTTGTTAATTTTCATCTTTTTTTTATCATAGTCCTCCCAACTGGGGTGATG
TTACCTCATTGTGGTTTTGATTTGCATTTCCCTGGTGATTGGTGACGTTGAGCATTTTTCATATACACTTGTTGGCC
ATCTGTATATCTTTTCTTGAGAAATGTCTACTCAGATAATTTGCCCATTTTTAAATGAGATTGGGTTTCTTTGCCAT
TGAGATGTATGAGTTCCTCGTATGTTCTGGATATGAATCACTTGTCAGATGAATAGCTGACAAATATTTTCTCCTAT
TCTGTAGGTTGCCTTTTCACTCTGTTGGTTGTTTCCTTTCTGCATAGAAGCTTTTTAGCTTGATATCATCTCATTTA
TTTACTTTTGCTTTTGTTGCTTGTGCTAGTGAGGTCTTACTCATAAAATATTTTTCCAGACCAATGTCCTAAAGCAT
TTCCCCTATGTTTTTTTCTAGTATTTTTTAAATTTTGTGTCTTATATTCAGGTCTTTGATCCATTTTGAATTGATTT
TTGTATAGGACGAGAGGTGTGAGTCTAATGTCATTCTTCTGCATATGGCACCAGTTTTCCCAGCATCATTTATTAAA
GAAACTGCTCTTTCCTCAATGAGTGTTCTTCATGCATTTGTCAAAATTCAGTTGGCTGTAGATCGTGGATTAATTTC
GGTGTTCTCTATTATGTATTATTGGTGTATGTATCTGCTTTTATGCCAATATCATGCTGTTTTGGTTACTACAGCTT
TGTAGTTTTGAAATCTTTAAATTTTTGAAATTTTGAAATTTTCTAGTTTTGAAATTTTGAAATCTTGTAGTGTGATA
CCTCCAGCTTTGTTCTTTTTTGCTTGGGATTGCTTTGACCATTCAGGCTATTTTTAGTTCCATATGAATTTTAAGAT
TGTTTCCTCTAATTCTGTGAAGAATTACATTGATATTTTGATAGAGCCAGGTTTGAATCTGTAGATTTCTTTGGGTA
GTATAATCATTTTAGCAATATTAATTCATCTGATGAGTAAGGAATGTCTTTCCATTTGTTTGTATCCTCTTCAGTTT
ATTTCCTCAGTGTTTTGTAGTTTTTCTTATTAAGGCTTGTCACCTCCTTGGTTAAATTTATTCCTAGGTATACTTCA
TTCTCTTATAGCTATTGTAAATGTGATTGCCTTCCTGATTTATTTTCAGCTAATTCATTGTGTGTAGAAATGCTACT
GATTTTTGTATATTGATTTTGCATCCTGCAAATTTACTAAATTCATTTATCAGTTCTGAGAGTTTTATTGTTAGAGT
CTTTAGGTTTTTGTTTTGTTTTGTTTTGTTTTGTTTTGTTTTTGAGATGGAATTTCACCATGTTGGCCAAGCTGGTC
TTGATCTCCTGGCCTCAAGCAATCTGCCCACTTTGGCCTCCTAAAGTGCTGGAATTACAGGCATGAGCCACCACGCC
TGGCCAAGTCTTTAGGTTTTTGTATGTTATTTGCAGAGACAATTTGACTTCCGCCTTTCCAGTTTGGATGGTTTTTA
TTTCTTTCTCTTGCCTAATTGCTCTGGCTAGGACTTTCAGTACTATGTAAAATAAGAGTCATAACAGTGGACATCCA
GTTCCTAGAGGAAAAGATTTCAGCTTTTCTCCATTCAGTATGATGTTAGCCATGGGTTTGTCATATATGGCCTTTTT
TGTGTTGAGGTACTTTCCTTCTATACCTAATTTATTGAGAGTTTCTATCATGAAACAATATTGAATTTTAACACATG
CTTTTTATTCTGCAACTATTTAGGTGATCATACGGTTTATGTCCTTCATTCTGTTGACATATGTATAACATTTATTG
ATTTGCATATGTTGAATCATTCTTGCCTTTCTGGGATTAATCCCACTTTATCATGGTATGTTATCTTTTTGATGTAT
TGTTGGATTTGATTTGCTACTATTTTGTTGAATATTTTTGCATCTATGTTCATCAGGGATATTGGCCTCTAGTTTTC
TTTTTTTATTGTCTCCTTTCTGATTTTGGTGTCATGGTTATGCTGGCCTTGTAGAATGAGTTAGGAAGAGTTGCCTC
CACTTCAATTTTTTGGAATAGTTTGAGAAGAGTTGGCATAATTTTTTTTTCTTTAAAGGTTCAGTAAAGTTCAGCAC
TGAAGCCATCCAGCCCTGGAATTTTCTTTGTTGGGGGGCCTTTTATTATTCATTCAATCTCATTACTTGTTGTTTGT
CTGCTGAAGTTTTCTATACCTTCTTGATTCAATCTCGGTAGATTATATGTGTCCAGGAACTTATCCATTTCTTCTAG
ACTTTCAAATTTGTTGGCATATTGTTCATAGTAGTGTCTAAGATCCTGTGTATTTCTGTGGTAACCATTGTGACATC
TTCTTTTTTATTTATGATTTTATTAATTTTTATGTCTTCTGTCTCTTTCTTAGTTTAGCTAATGATTGTCAATTTTA
TTTATTTTTCCAAAAAGCGAACTTGTTCATTGATTTTTTTTTAATTTCATTTATTTCTGCTCTGATCTTTATGATTT
CTTTCATTGTGCTGATTTTGGATTTGGTTTGTTCTTGCTTTCTAGTTTCTTGAAATGCACAGTTAAATGGTTTACTT
GAAATTTGTCTAATTGTTTGATGTAGGCATTTATTTCTCTCAAGTTGTCTCTTAAAACTGTTTTTGCTGTGTCCCAT
AGGTTTTGGTATATTTTATTTCTATTTTTATTTATTTTGAGAAATTTTTAAATATCATTCTTAATTTCTTCCTTCAC
TATTGGTCATTTAGAATCATTTTGTTTCATTTCTGTGTATTTGTATAGTTTGCATGTTTCCCTTGGTATTGATTTTT
AGTTTTATTCAATTGTAGTCAAATAAGATACTTGATACATTTTGGTTTTTAAAAATTTTTGGCACTTGTTTTGTGTT
CTAACATATGGTCGATCCTTGGGAATGTTGCACATGCTGATGAAACCATGTGTATTCTGCAGCTGTCGGTTGAAATG
TTCTGTAAATATCTTAGGTTCATTTGGTATATGGTGCAGTTTAAATCCAACGTTTATTTGTTAATCTTGTCTAGATG
ATTTGTTCAATGCTGAGAGTGGGGCGTTGAAGTCCTCAACTATTATTGTATTGGAGTCTATCTCTCCCTTTATATCT
AATAATATTTGCTTTACATATCTGGGTGCTCTGGTGTTGTGTGCATATGTATTTACAGTTGTTATATTATAGTGCTG
AACTGACCCCTTTATAATAATATAATGTCCTTCTTTGTCTCTTTACAGCTTTTGACTTGTAGTCCGTTTTGTCTGAG
ATAAGTATAGCTATTCCTGCTTCCTTTCATTTCCACTTGGGTAGAATATCTTTTTCCATCTCTTCCTTTTCAGTCTA
TGTGTGTCTTCTAGGTGAGATAAGTTTCTTGTAAGCAGTATATAGCTGTGTTGGTAGAAGGGCTGAGGCAGGGCTTG
CTTGTCTGACATAATGTAAAAGAGTCTTGGAACATGTCCTGGGTCCAGGGTCTCAAACCCCTCGTGGCCTATGGAAC
ACCAAGCTCTGTGCCTAAGGGTGGAAGGCTGCCCTGCCACACTGCAATCTAAGCCCAGGGCATAAAACCCCTCGTGG
CTTGGAAAGAATCCAGGGCTCTGGGCATAAAACCCCTCATAGCCTCTGGAATGTGTCCAGACTTGCTGGCCCCTTGC
TCCTTGCTCTCCCAGGATCATAAATTGATTGTATCTTGAGTGAAAAGAACTTGTTCTCCATTATTTCAAGTAGCAGA
GCATATGCTAAACCGTCACAGCTATGCTTGATGCACCGCTACCTTTCTACCCCAAAGTCCTCACGTTCTCACTTGTC
TATCCCCACTTCTGCACGTCCTCACCACCTGCTTCTTTGTTTGATTACCAATAAATAGTGTGGGCTCCCAGAGCTCG
GGGCCTTCACAGCCTCCATACTAGCGTCGGCCCCCTGGACTCACTTTATGTACTATTAACTTGTCTTGTCTCATTCC
TTTGACTCCGCTGGACTTCGTGGCCCCCACGGCCTAGTGTTGGATCTGATCACCCCAACAAGCTGAGTCTAGATTTT
CTTTTCATTCATTCAGGCAGTCCATATATTTTAAATGGGACAATTTAATCCATTTACATACACATTATTATTAATAG
GTTATTTTCATTTCATTGATTGTTTTCTGATTGTTTTATATATTCCTGGTTCCTTACTTCCCCTCTTATTGTTTCTT
TTTGTGGTTGGCTGATGTTTTTTTTTTTTTTGTAGTGATAAGATTTGATTCCTTTCTCTTTCTTCTTTGTGTATGGG
CTGTCAGTGAGTTTTAAGTTCACGTGTTTTTGCCTTTTCACTTCCAGATGTAAGACTCCCTTGAGCATTTCTTTTCT
TTTTCTTCTCTTATTTATTTTTATTATTTTTTTTTTGAGAAAGTGTCTCACTCTGTCGCCCAGGCAGGAGTGCAGTG
GCATGATCACGGCTCACTATAGTCTCGACCTCCTGGGCTTAAGCAATCTTCCTGCCTTAACCTCCCAAGTAGCTGGG
ACTACAGGCATGTGCCACCACGCCCAGCTAATTTTTGTGTTTCTTGTAGAGGTAGGGTGTTGCCATTTGCCTAAGCT
GGTCTCAAATTAAAGAGCTCAAGTGGTCCACCTGCCTGCCTTCACCTCCCAATGTGCTGGGATTATAGGCATGAGCC
ACACTGTGCCTGGCCCCTTGAGCATTTCTTGTAAGGCCAGTCTAAGAGTGATTAGAATTCCCTTAGTTTTTGCTTAT
CTATGAAATATTTTATTTCTCCTTCTTTTCTGAAAGATAGCTTTTCTGGGTATAGTATTTTTGACTGTTAAGTTTTT
TATCTTTCAGTACTTTGAGTATGTCATCCCATTCTATCCTGGCCTATATAATGTTACTGCTGAGAAACTCACTGTTA
GTCTAATAAGGATAATCCTATATGTGACTAGATACTTTTACCTTGCTGTTTTTACAATTCTTTACTTGACTTTTGAC
AATTTGGCATAATGAGCTTTGGAGAGGACTTGCTTGGGTTGAATATTTTGAGAGTACTTTGAGCTTCCTGGACCTGG
ATGTCCTTCTAGTTCCCAAGGCTTGGGAAGTTTTCACCTATTACTGGATTAAATATGTTTTCTACACCTTTTCCATT
CTCTTCTCCTCCTGGAAATACCATAATGTGAATATTTGCTTGATTGTGTCCCATGAGTCCTGTAGGTTTCCTTCGTT
CTATTTTATTCTCTTATTTTTACCTGCCTGTGTTATTTCAGAAGATCTGTCTTCAAGTTCAGAAATTATTTTTTCTT
CTTGACCTAGCCTGTTGTTGAAGCTCTCGATTGCGGTTTTTTATTTCATTTATTGAGTTCTCAGCTGTAGGAGTTCT
GCTTTGTTCTTTTATATAATATCTATCTCTCTGTTAAATTTCTCTTTCAAGTCATGAATTAAAACAATGGGACACAG
GTGCCCAACTACTTGGCTGACCTGGGGGCATATCTGCTGGAGGTGCCAACATGGCTGTTTTGCAGGGCTGAGATGAA
GCTGAATGACTCTTGGCTGGCCTAGGTGTGTTTTTGCCAGGAGTAGCACTCAGAGCTTTATCTAGGGTTTGGGATGT
GAGTGTAAGACTGCTCAGCTGGCCTAGGGGGTGTACCAGCCAGTGGTAGCCCATGGGGCTGTTTCTCAGGCCTGGAA
TGCAAGCACATTCTGCCTGGGGTCATGTCTAAAAGGGTTGGCTCACAAGGCTGTTTCTCAGGCCCTAATTGTGGGAG
AGTGGCCTTTGGGCAGGCCAGAGTCATGTCCACAGAAGGCGTCTGGGCACCGTAAGGCTGTTTCTCAGAGCCTGTGT
GTGAGCACATAACCACTACCCCAGCCTGGGGATGTATCAACTCTTTGTTGGCTCAGAGGTCTCTCCCATTCAGGTGA
GCATGCACAGTAGTTTGGCCAACTCAATTGTGTGTTCGCCCTGAGTGGGACTATAAGACCTTTCCTCCAGCTGGAAG
TACGGGCAGCAGGGGTTGGTTTCTCTGCTGTTCAGGGCCAGAGTCCCAGCCAATCCTGGGCCCAGGCTCCATGCAGC
TCTAATTGTGGTATTCAGCCACTACTGCAGGTTTAGTGGAATGAAGATGCACAATGATAAAGAGGTGCATGCCACTG
GCCCCCAGAGGAGGGTGCACTCCAGAGATGGCTGTGGTCTCAAGATGGTTCTGTGTTGTAGCAGCTTGCCCGCAGGG
GCTGGTTAGGGAGTTGGGAGTGCACACCAAATGCTCCATGCAGCTGTGTGAATTCCTGGCAGCTCTTCCAACTGTGC
TCAGAGCTTGTGAGGACTGTAAGATTAACCTGTAGTAAGGAATGTAGGTATCTGCAGTGGCACTGGAGGTTGGTTGG
ATTCCTCTGCTTATCATTTCCCTACAAGGGGAAATCCTTCCTGTCTCTGGGACAAACCAATCTGGGCTGGGGAGATG
GAGCTGCAAAGCCCGGGTGCCTCCATGCTGCCCTCCTGGGTTTCCAATTACCACAGGTAACTCTCCACTCCCTTGCT
GCACTACACTACTCTCCCTTCGACACTCCACTCAAATCTTTGCTGTGGTTTATTCATTGCCTTGGTCCTTTCTTGTC
TGGTGACACGGGGGAGGATGAGCTCCAGGCACCTCCGGTGAGCCATTTTGCTCCAATGGGGGCATTTTTTTTTAATA
GGTTTTATTTTTCAGAGTAGTTTTTGTTTCACAGCAAAATTGAGTGGAATCTTCTAGTCGCTGATCATCTTGGGAGC
ATTTATAAATGAACCTTATTTTTCATGAAGAAATTGAGCAGAAGATACTAAGACTTCCCGTATGCCCTCTACCCTTA
CACATAGTTTCCCCGGCCATCAGCATCCCCCATCAGAGTGGTACATTTGTTACAGTCAATAAAACTACATTGACATA
TCATTGTCACCTGAAGCCCATAGTTTACATTAAAGTTCACTCTTGGTGTTGTACATTTTACAGGCTTTTTAAAAATG
TATAATGACATGAATCCACCATGAGAGTATCATATAGAATAGTCACACTTCCCTAAAAATCTCTTTAGGGCATTTTT
TTCTACTGTCCATACCTCAACCCTTAGCCCTGGCCTCTGTCCAAAGACCAGTGCTCTCTCCACTGCCCTATTCCAAT
TAATAATGGCATCTGGCACCTCAGTGGACAGTGAGCCCAGTGAGAGCAGGAACAGTTCCCTCAGTAGTGGTTATCAA
ACTGTTAACAATGATGCTCAGAGACACGCCCCTGACTCTGAGTGTTGGGACCTAGAAGGCACAGCCAGGCAGGTCCA
GGAGAACTGTCTGGGTCTAAGAAGGTCTGAGAACCACCTCCCTGCCCCACCCTGCTTCCAGGCCCTTTTTAAGGCCA
AAAGGACCACCTTTGACCCTAAGTGATGGGGCCAGTGGGAAGAAAGAAGAGACAAGGCCTATCAGCATTCCAGTGCT
TTCTCTCTCTCTCATCCAAGAGGCTCAGAGCTTCACAGTCCTTCAGGGGCTATGTCTGAGGTTCATTTCAGAAAGAC
CCAGGGTGGAGAGGAACCTGAGTCCTAGGAGAGATGATGTTTTGTGCACCAGAGAGAGAGGGTGGGACAAGAGGTGT
CAGGTGCACTGTGTACTTCATCTCATGGTCGTGGTCAATATTGATGTCTATGATGGGTGGGAAGATCTAGGAGCTAA
ACCCCATTTTGGAGGTGAAGTCACCCCTCTCTACATGCTGGAGAGGAGGATACACATACCTGTTTATCTAGATTAGA
ATTCACCCCAAATCTTTTTTGTCTGCAGGGAAACAAGAGACCAGAGCAGGAGTGGTTCATGGGGCCATTGGAGGAGC
TGGTGTTACAGCCCTGCTCGCTCTTTGTCTCTGCCTCATCTTCTTCATGTGAGCATTTTCTCTGGGTCAGGCATGGG
CCAGAGGTGAAGAGGATGGACCTGGTGTAGAAGGGTCCTGGAGGGGCTGTGAGGGCTGGAGAAAGGGCAGGGGGTGT
GATGATGTACAGAATCCAGCCTGTGGCCACTGGGATAGGCGTGGGTCTATTCCAGGGCCCTGATCTCAGATGTCCAA
GGAGTGGGAGGTAGAGGGAGACCTTGTGACTAAGTCTTGTTTGAGGGCTCCTGGATTAATCCCACCCTTTACCTGCC
AAAGTCCCTCATTCCAGGCTCATAACAATGGCCCCACAGCCTGAGAAAACCAGGCTCAAAGACCCTGGTGTCTCCCA
TCAGAGTGAAGACCCACAGGAGGAAAGCAGCCAGGACAGCAGTGGGCAGGAATGACACCCACCCTACCACAGGGTCA
GCCTCCCCG (SEQ ID NO: 15)
A Gly-Ser linker
GGSGGS (SEQ ID NO: 16)
CD33^{ΔE2}
MPLLLLLPLLWADLTHRPKILIPGTLEPGHSKNLTCSVSWACEQGTPPIFSWLSAAPTSLGPRTTHSSVLIITP
RPQDHGTNLTCQVKFAGAGVTTERTIQLNVTYVPQNPTTGIFPGDGSGKQETRAGVVHGAIGGAGVTALLALCL
CLIFFIVKTHRRKAARTAVGRNDTHPTTGSASPKHQKKSKLHGPTETSSCSGAAPTVEMDEELHYASLNFHGMN
PSKDTSTEYSEVRTQ (SEQ ID NO: 17)
Linker: (SGGS)₂-XTEN-(SGGS)₂ (SEQ ID NO: 18)
CD33E2 splice gRNA for CD33 knockdown: CCCCACAGGGGCCCUGGCUA (SEQ ID NO: 19)
CD33 exon 2 splice acceptor site 2 gRNA for CD33 knockdown:
CCCACAGGGGCCCUGGCUAU (SEQ ID NO: 20)
CD33 exon 3 splice acceptor site 5a gRNA for human CD33 knockdown:
CCUCACUAGACUUGACCCAC (SEQ ID NO: 21)
CD33 exon 3 splice acceptor site 5b gRNA for rhesus CD33 knockdown:
UCUCACUAGACUUGACCCAC (SEQ ID NO: 22)
FV Pol gene DNA
To generate integration deficient foamy vector, either bolded A is mutated
to C in underlined sequence or underlined A is mutated to C in bolded
sequence
ATGAATCCCCTCCAACTGTTGCAGCCTCTGCCCGCAGAGATCAAAGGGACTAAACTGCTGGCTCATTGGGACTC
TGGAGCAACCATAACATGCATACCAGAAAGCTTCCTTGAGGACGAGCAGCCTATCAAAAAAACATTGATTAAGA
CGATCCACGGGGAAAAGCAGCAGAACGTGTATTACGTTACCTTTAAGGTGAAGGGCCGGAAAGTCGAGGCCGAG
GTCATTGCCTCTCCATACGAATACATTCTGCTCTCACCCACCGACGTGCCATGGTTGACCCAGCAGCCTCTTCA
GCTGACTATCCTGGTCCCTTTGCAGGAGTACCAGGAAAAGATTCTGAGCAAGACGGCGCTTCCCGAAGATCAGA
AACAGCAGCTGAAGACCCTCTTCGTGAAATACGATAATCTCTGGCAGCACTGGGAAAACCAGGTGGGCCATCGG
AAGATTCGACCCCACAATATCGCCACGGGCGACTATCCACCTAGGCCTCAGAAGCAGTATCCCATCAACCCAAA
AGCAAAACCAAGCATCCAGATCGTCATCGATGATTTGCTTAAGCAAGGAGTGCTCACCCCACAAAATAGCACTA
TGAACACCCCAGTGTACCCCGTGCCCAAACCGGACGGCAGATGGAGAATGGTATTGGACTATCGCGAAGTTAAC
AAAACCATACCTTTGACCGCAGCCCAGAATCAACACAGCGCCGGCATCTTGGCTACGATCGTGAGACAGAAGTA
CAAAACAACTCTCGATCTGGCCAACGGCTTTTGGGCTCACCCAATCACTCCAGAGAGCTACTGGCTTACCGCCT
TTACATGGCAGGGGAAACAATACTGTTGGACCCGGCTGCCTCAGGGGTTCTTGAATTCACCCGCACTGTTTACA
GCTGACGTCGTTGATCTGCTGAAAGAAATCCCCAATGTGCAGGTATACGTGGACGACATCTATCTTTCCCACGA
CGATCCAAAAGAGCATGTTCAGCAGCTCGAAAAAGTTTTCCAGATCCTGCTGCAGGCTGGTTATGTCGTCTCAC
TCAAGAAGTCTGAGATAGGACAAAAGACTGTGGAGTTTCTGGGATTTAACATCACCAAGGAAGGACGGGGATTG
ACTGATACGTTCAAGACTAAGCTGCTCAACATTACTCCTCCCAAGGATCTTAAGCAGCTGCAGAGTATTCTTGG
CTTGCTCAATTTTGCCCGGAATTTTATCCCTAACTTCGCTGAGCTTGTTCAGCCCCTGTATAATCTGATAGCCT
CCGCCAAGGGTAAGTACATCGAATGGAGCGAGGAGAATACTAAACAGTTGAACATGGTGATTGAGGCACTTAAC
ACTGCCTCCAACTTGGAGGAACGACTGCCAGAGCAGCGACTTGTGATTAAAGTGAACACCTCACCAAGTGCGGG
GTACGTGCGCTACTACAACGAGACAGGCAAAAAGCCCATAATGTACCTGAACTATGTCTTCTCAAAAGCTGAGC
TCAAGTTTAGCATGCTCGAGAAGCTGCTTACTACCATGCACAAGGCCCTGATAAAGGCCATGGACCTTGCCATG
GGGCAAGAAATCCTCGTGTACAGCCCCATCGTTTCCATGACGAAGATCCAGAAAACACCACTGCCCGAACGAAA
GGCCTTGCCTATCAGATGGATTACTTGGATGACCTACCTTGAGGACCCCCGCATCCAGTTTCATTATGATAAGA
CCCTGCCTGAACTGAAACACATCCCAGACGTGTACACCTCCAGTCAGTCCCCAGTCAAGCACCCTTCTCAATAT
GAAGGAGTGTTTTATACCGATGGGAGTGCCATCAAATCCCCTGACCCCACAAAAAGTAACAACGCCGGTATGGG
TATCGTCCACGCGACCTATAAGCCCGAGTATCAGGTACTGAACCAGTGGTCCATCCCGCTGGGGAATCATACCG
CCCAGATGGCGGAAATTGCCGCAGTCGAGTTTGCCTGCAAAAAGGCATTGAAAATCCCAGGGCCTGTCCTGGTC
ATCACCGACTCTTTCTACGTAGCCGAGTCAGCCAATAAGGAACTGCCCTATTGGAAAAGTAATGGCTTCGTGAA
CAACAAGAAGAAGCCACTGAAACATATTAGCAAATGGAAATCTATTGCCGAGTGTCTGTCTATGAAGCCCGACA
TCACTATCCAGCACGAAAAGGGCCATCAGCCCACCAACACTAGTATCCATACGGAGGGAAACGCTCTGGCCGAT
AAGCTAGCCACTCAAGGGAGTTACGTCGTGAACTGCAACACCAAGAAACCTAACCTTGACGCCGAATTGGACCA
ATTGCTGCAGGGACATTACATAAAGGGCTACCCCAAGCAGTATACCTATTTTCTGGAAGACGGCAAGGTAAAAG
TGTCCCGGCCAGAGGGCGTCAAGATCATCCCGCCACAAAGCGACAGACAGAAAATCGTTCTGCAGGCCCACAAC
CTCGCTCATACTGGGCGCGAAGCTACTCTGCTCAAGATTGCCAATCTGTATTGGTGGCCGAATATGAGAAAAGA
CGTCGTAAAGCAACTGGGGCGCTGTCAGCAGTGTTTGATCACTAACGCAAGTAACAAAGCAAGTGGGCCGATTC
TTCGACCAGACCGCCCTCAGAAACCGTTCGATAAGTTTTTTATAGATTACATTGGACCTCTGCCTCCCAGTCAA
GGCTACCTCTACGTGCTGGTAGTGGTCGATGGCATGACGGGATTCACATGGCTGTACCCGACCAAGGCGCCGAG
TACTTCCGCGACGGTCAAGAGCCTTAACGTTCTCACCTCCATAGCTATCCCCAAAGTTATCCACTCCGACCAGG
GCGCAGCTTTCACCAGCTCTACCTTCGCGGAGTGGGCCAAAGAGAGGGGGATTCACTTGGAATTCTCAACGCCT
TACCACCCCCAATCTAGCGGAAAGGTCGAGAGAAAAAATTCAGATATCAAAAGACTGTTGACCAAGCTGCTTGT
TGGCCGCCCTACAAAGTGGTATGACCTCCTGCCTGTCGTCCAGCTGGCACTGAACAACACCTACAGCCCCGTGC
TCAAGTATACACCTCATCAGTTGCTGTTTGGTATTGATAGTAACACTCCTTTCGCAAATCAGGATACGTTGGAT
CTCACTCGCGAAGAAGAGCTCAGTTTGCTGCAGGAGATACGCACGAGTCTGTACCACCCTTCCACTCCTCCCAC
TTCTAGTAGGTCTTGGTCTCCAGTTGTGGGACAGCTTGTTCAGGAAAGAGTCGCCCGGCCCGCATCACTGCGGC
CCCGGTGGCACAAACCGTCTACTGTACTGAAGGTGCTCAACCCACGGACGGTGGTAATCCTTGACCATCTCGGA
AACAACCGGACAGTGTCAATCGATAACCTCAAGCCAACCTCCCACCAAAACGGCACAACCAATGACACAGCCAC
AATGGATCATTAG (SEQ ID NO: 23)
FV Pol gene AA
To generate integration deficient foamy vector (IDFV), underlined D is
mutated to A (separately, 2 different versions)
MNPLQLLQPLPAEIKGTKLLAHWDSGATITCIPESFLEDEQPIKKTLIKTIHGEKQQNVYYVTFKVKGRKVEAE
VIASPYEYILLSPTDVPWLTQQPLQLTILVPLQEYQEKILSKTALPEDQKQQLKTLFVKYDNLWQHWENQVGHR
KIRPHNIATGDYPPRPQKQYPINPKAKPSIQIVIDDLLKQGVLTPQNSTMNTPVYPVPKPDGRWRMVLDYREVN
KTIPLTAAQNQHSAGILATIVRQKYKTTLDLANGFWAHPITPESYWLTAFTWQGKQYCWTRLPQGFLNSPALFT
ADVVDLLKEIPNVQVYVDDIYLSHDDPKEHVQQLEKVFQILLQAGYVVSLKKSEIGQKTVEFLGFNITKEGRGL
TDTFKTKLLNITPPKDLKQLQSILGLLNFARNFIPNFAELVQPLYNLIASAKGKYIEWSEENTKQLNMVIEALN
TASNLEERLPEQRLVIKVNTSPSAGYVRYYNETGKKPIMYLNYVFSKAELKFSMLEKLLTTMHKALIKAMDLAM
GQEILVYSPIVSMTKIQKTPLPERKALPIRWITWMTYLEDPRIQFHYDKTLPELKHIPDVYTSSQSPVKHPSQY
EGVFYTDGSAIKSPDPTKSNNAGMGIVHATYKPEYQVLNQWSIPLGNHTAQMAEIAAVEFACKKALKIPGPVLV
ITDSFYVAESANKELPYWKSNGFVNNKKKPLKHISKWKSIAECLSMKPDITIQHEKGHQPTNTSIHTEGNALAD
KLATQGSYVVNCNTKKPNLDAELDQLLQGHYIKGYPKQYTYFLEDGKVKVSRPEGVKIIPPQSDRQKIVLQAHN
LAHTGREATLLKIANLYWWPNMRKDVVKQLGRCQQCLITNASNKASGPILRPDRPQKPFDKFFIDYIGPLPPSQ
GYLYVLVVVDGMTGFTWLYPTKAPSTSATVKSLNVLTSIAIPKVIHSDQGAAFTSSTFAEWAKERGIHLEFSTP
YHPQSSGKVERKNSDIKRLLTKLLVGRPTKWYDLLPVVQLALNNTYSPVLKYTPHQLLFGIDSNTPFANQDTLD
LTREEELSLLQEIRTSLYHPSTPPTSSRSWSPVVGQLVQERVARPASLRPRWHKPSTVLKVLNPRTVVILDHLG
NNRTVSIDNLKPTSHQNGTTNDTATMDH* (SEQ ID NO: 24)
PGK promoter
GGGGTTGGGGTTGCGCCTTTTCCAAGGCAGCCCTGGGTTTGCGCAGGGACGCGGCTGCTCTGGGCGTGGTTCCG
GGAAACGCAGCGGCGCCGACCCTGGGTCTCGCACATTCTTCACGTCCGTTCGCAGCGTCACCCGGATCTTCGCC
GCTACCCTTGTGGGCCCCCCGGCGACGCTTCCTGCTCCGCCCCTAAGTCGGGAAGGTTCCTTGCGGTTCGCGGC
GTGCCGGACGTGACAAACGGAAGCCGCACGTCTCACTAGTACCCTCGCAGACGGACAGCGCCAGGGAGCAATGG
CAGCGCGCCGACCGCGATGGGCTGTGGCCAATAGCGGCTGCTCAGCGGGGCGCGCCGAGAGCAGCGGCCGGGAA
GGGGCGGTGCGGGAGGCGGGGTGTGGGGCGGTAGTGTGGGCCCTGTTCCTGCCCGCGCGGTGTTCCGCATTCTG
CAAGCCTCCGGAGCGCACGTCGGCAGTCGGCTCCCTCGTTGACCGAATCACCGACCTCTCTCCCCAG (SEQ
ID NO: 25)
SV40 promoter:
GGTGTGGAAAGTCCCCAGGCTCCCCAGCAGGCAGAAGTATGCAAAGCATGCATCTCAATTAGTCAGCAACCAGG
TGTGGAAAGTCCCCAGGCTCCCCAGCAGGCAGAAGTATGCAAAGCATGCATCTCAATTAGTCAGCAACCATAGT
CCCGCCCCTAACTCCGCCCATCCCGCCCCTAACTCCGCCCAGTTCCGCCCATTCTCCGCCCCATGGCTGACTAA
TTTTTTTTATTTATGCAGAGGCCGAGGCCGCCTCTGCCTCTGAGCTATTCCAGAAGTAGTGAGGAGGCTTTTTT
GGAGGCCTAGGCTTTTGCAAAAAGCTCCCGGGAGCTTGTATATCCATTTTCG (SEQ ID NO: 26)
dESV40 promoter:
GCATGCATCTCAATTAGTCAGCAACCATAGTCCCGCCCCTAACTCCGCCCATCCCGCCCCTAACTCCGCCCAGT
TCCGCCCATTCTCCGCCCCATGGCTGACTAATTTTTTTTATTTATGCAGAGGCCGAGGCCGCCTCGGCCTCTGA
GCTATTCCAGAAGTAGTGAGGAGGCTTTTTTGGAGGCCTAGGCTTTTGCAAAAAGCTT (SEQ ID NO: 27)
Human telomerase catalytic subunit (hTERT) promoter:
TTGGCCCCTCCCTCGGGTTACCCCACAGCCTAGGCCGATTCGACCTCTCTCCGCTGGGGCCCTCGCTGGCGTCC
CTGCACCCTGGGAGCGCGAGCGGCGCGCGGGCGGGGAAGCGCGGCCCAGACCCCCGGGTCCGCCCGGAGCAGCT
GCGCTGTCGGGGCCAGGCCGGGCTCCCAGTGGATTCGCGGGCACAGACGCCCAGGACCGCGCTCCCCACGTGGC
GGAGGGACTGGGGACCCGGGCACCCGTCCTGCCCCTTCACCTTCCAGCTCCGCCTCCTCCGCGCGGACCCCGCC
CCGTCCCGACCCCTCCCGGGTCCCCGGCCCAGCCCCCTCCGGGCCCTCCCAGCCCCTCCCCTTCCTTTACCGCG
GCCCCGCCCTCTCCTCGCGGCGCGAGTTTCAGGCAGCGCTGCGTCCTGCTGCGCACGTGGGAAGCCCTGGCCCC
GGCCACCCCCGCCAGATCT (SEQ ID NO: 28)
RSV promoter derived from the Schmidt-Ruppin A strain:
acgcgtcatgtttgacagcttatcatcgcagatccgtatggtgcactctcagtacaatctgctctgatgccgca
tagttaagccagtatctgctccctgcttgtgtgttggaggtcgctgagtagtgcgcgagcaaaatttaagctac
aacaaggcaaggcttgaccgacaattgcatgaagaatctgcttagggttaggcgttttgcgctgcttcgcgatg
tacgggccagatattcgcgtatctgaggggactagggtgtgtttaggcgaaaagcggggcttcggttgtacgcg
gttaggagtcccctcaggatatagtagtttcgcttttgcatagggagggggaaatgtagtcttatgcaatactc
ttgtagtcttgcaacatggtaacgatgagttagcaacatgccttacaaggagagaaaaagcaccgtgcatgccg
attggtggaagtaaggtggtacgatcgtgccttattaggaaggcaacagacgggtctgacatggattggacgaa
ccactaaattccgcattgcagagatattgtatttaagtgcctagctcgatacaataaacgccatttgaccattc
accacattggtgtgcacctccaagctgggtaccagctgctagcaagcttgagatct (SEQ ID NO: 29)
hNIS promoter:
gagtagctgggattacaggcatgtgccaccacgcctcgctaatattagtatttttcatacagacaagatctcac
tatgttgctcagggtagtctcgaattctgggactcaaatgatcctcccacttcagcctcccaaagtgctgggat
tacaggcataagccatcatgcccggcctctgacgctgtttctttcaacccccaggatttcagattccaccagct
tatggagaagggaaccaagttcgagatgcgtgattgcccagaaagttggaggctgagctgagacttgaacccag
agaccagaacctccagaggtcaaagtcctcctcctgggtcccccagagaagggccctgagatgacagctcgttg
gtcctcatggaagcgtgacccccccagtagactttctcccacacccaaccttggtttcctcatctatatgatag
ggacaagccagactctacctccctggtggtcatggtctccgcttattcgggttcataaccttaaaggcccctcg
caccacctcagtgagccatttatgcctggcacagggccaactctcagtgcatatctgcaaaggaaccaatgaat
gagtgaatgaagtgacaaatgaataaaggaataaatgaatgaggcacttatcatgtaccaggctttcgttacca
cgtcccatttattcctctgaggcagggtctattttatccttgttacagatggggaaactaaggcccagggagga
gcaaagtcttccccaagtatgtacccactcagaacttgagctctgaatgtctcccacccagcttagcccaagag
cggggttcagtgatgcccaccccctaaggctctagagaaagggggtaggcccacatgccagtttgggggtggta
aagccaggtaagttttctttatgggtcccctgaaaccctgaaagtgaaccccagtcctgcatgaaagtgagctc
cccatagctcaaggtattcaagcacaatacggctttgagtgctgaagcaggctgtgcaggcttggatagtgaca
tgccctctctgagectcaatttccccacctgtcaacagcagacagtgacagctgtgatcaggggatcacagtgc
atggggatgggtgggtgcatggggatggaggggcatttgggagccctccccgataccaccccctgcagccaccc
agatagcctgtcctggcctgtctgtcccagtccagggctgaaagggtgcgggtcctgcccgcccctaggtctgg
aggcggagtcgcggtgacccgggagcccaataaatctgcaacccacaatcacgagctgctcccgtaagccccaa
ggcgacctccagctgtcagcgctgagcacagcgcccagggagagggacagacagccggctgcatgggacagcgg
aacccagagtgagaggggaggtggcaggacagacagacagcaggggcggacgcagagacagacagcggggacag
ggaggccgacacggacatcgacagcccatagattcctaacccagggagccccggcccctctcgccgcttcccac
cccagacggagcggggacaggctgccgagcatcctcccacccgccctccccgtcctgcctcctcggcccctgcc
agcttcccccgcttgagcacgcagggcgtccgaggacgcgctgggcctccgcacccgccctcatggaggccgtg
gagaccggggaacggcccaccttcggagcctgggacta (SEQ ID NO: 30)
Human glucocorticoid receptor 1A (hGR 1/Ap/e) promoter:
ATTAGAGATTGTAAATTGGGCTCTGAGCTTCCTACCAACAAAAGCACAAAGGAAAATATGATCACTGGTATTAA
AAAAAAACACCTATGGTTTCCAAAAGATTAAAACAAACCAGCAGTTTTATAGAAGCTAACACTAAAATCTAAAG
GAACTACGTTCTATGGAGCCACTTAATATGGATAAACACTTTGACAATATTCTTTCAACAACTACAGTAACAAG
TTTCTTAGAGTCCATTTCTTTTTACATCCATAATGAATTGTAAATCTTTTCTACTTCTTAAGTAAAACATCACC
ACTTAATTCTGGTAACTTTTCCATATTAACTTTTTAGAACAATTGCAAACGTACCATAAATGATTGTTGTCACA
GTGGTAACTATTTGACCCTGACTGTTATTTTGTATATAGCAGCTTTTAAAATAAAAAGGCAACAAGTTTCTAGG
CGTAATTTCCACAGATCTTTTATGTAAAACAATGACATCCTTTGCAACTTCTGCCATTTAATCTATCTCAAGCA
AGCTCTCTGGAAACAAATCTATTTGAAAGATTCTATTGTAATTAGAAATCAGGGTAACTGAATGCACTAGATGA
AAACCTTCTGACTGGGGCCAATGAAGTCAATAAAGTCAAAACTGCTGTGAATGCTCAACTGTCTGCAGATCAGA
TGTCTTGGGATGGAATCCGTTCTCGAGGCCACCATCATTAATATCAATTTGGCCATGTAATACAAGCCTCACTT
GTTCCACTGTTACAAATGTGCTTAAAACTGAGCTCATTTACAATCCAAATACATATGTAGGATGGTAACCAAGG
CATCACACTAATTTAGGTATTATGTTTTAGGGGGAACAAAAGGTATGTTAATATTTTATTCATCTCCAAATTAA
CTATAAATTGTGCATTCTTGCATAGATCCTCCTTGGGAATGAGAAATTAGGAAAATCCAGTTGTTAAAATGAAT
GCCTAAAATCAAAATAAAATTTGTTTTTCTGGCACCTGCTTGATGACACAGACTAATAACCAATGACAAAATTC
CCTTGAACCCAAGTTTTCATTTCCTCCTATTGTGTGGTC (SEQ ID NO: 31)
Light chain of hP67.6:
MSVPTQVLGLLLLWLTDARCDIQLTQSPSTLSASVGDRVTITCRASESLDNYGIRFLTWFQQKPGKAPKLLMYA
ASNQGSGVPSRFSGSGSGTEFTLTISSLQPDDFATYYCQQTKEVPWSFGQGTKVEVKRT (SEQ ID NO: 32)
Heavy chain of hP67.6 includes:
MEWSWVFLFFLSVTTGVHSEVQLVQSGAEVKKPGSSVKVSCKASGYTITDSNIHWVRQAPGQSLEWIGYIYPYN
GGTDYNQKFKNRATLTVDNPTNTAYMELSSLRSEDTDFYYCVNGNPWLAYWGQGTLVTVSSASTKGP(SEQ
ID NO: 33)
CDRL1 of hP67.6 binding domain: QSPSTLSASV (SEQ ID NO: 34)
CDRL2 of hP67.6 binding domain DNYGIRFLTWFQQKPG (SEQ ID NO: 35)
CDRL3 of hP67.6 binding domain: FTLTISSL (SEQ ID NO: 36).
CDRH1 of hP67.6 binding domain: VQSGAEVKKPG (SEQ ID NO: 37)
CDRH2 of hP67.6 binding domain: DSNIHWV (SEQ ID NO: 38)
CDRH3 of hP67.6 binding domain: LTVDNPTNT (SEQ ID NO: 39)
CDRL1 of h2H12EC binding domain: NYDIN (SEQ ID NO: 40)
CDRL2 of h2H12EC binding domain: WIYPGDGSTKYNEKFKA (SEQ ID NO: 41)
CDRL3 of h2H12EC binding domain: GYEDAMDY (SEQ ID NO: 42)
CDRH1 of h2H12EC binding domain: KASQDINSYLS (SEQ ID NO: 43)
CDRH2 of h2H12EC binding domain: RANRLVD (SEQ ID NO: 44)
CDRH3 of h2H12EC binding domain: LQYDEFPLT (SEQ ID NO: 45)
Light chain of a representative anti-CD33 antibody:
NIMLTQSPSSLAVSAGEKVTMSCKSSQSVFFSSSQKNYLAWYQQIPGQSPKLLIYWASTRESGVPDRFTGSGSG
TDFTLTISSVQSEDLAIYYCHQYLSSRTFGGGTKLEIKR (SEQ ID NO: 46)
Heavy chain of representative anti-CD33 antibody:
QVQLQQPGAEVVKPGASVKMSCKASGYTFTSYYIHWIKQTPGQGLEWVGVIYPGNDDISYNQKFKGKATLTADK
SSTTAYMQLSSLTSEDSAVYYCAREVRLRYFDVWGAGTTVTVSS (SEQ ID NO: 47)
CDRL2 for CD33 binding domain: VIYPGNDDISYNQKFXG (SEQ ID NO: 48) wherein X
is K or Q
CDRL3 for CD33 binding domain: EVRLRYFDV (SEQ ID NO: 49)
CDRH1 for CD33 binding domain: KSSQSVFFSSSQKNYLA (SEQ ID NO: 50)
CDRH2 for CD33 binding domain: WASTRES (SEQ ID NO: 51)
CDRH3 for CD33 binding domain: HQYLSSRT (SEQ ID NO: 52)
Gly-Ser Linkers:
(GGGGS)n (SEQ ID NO: 53)
(GGGS)n(GGGGS)n (SEQ ID NO: 54)
(GGGS)n(GGS)n (SEQ ID NO: 55)
(GGGS)n(GGGGS)₁ (SEQ ID NO: 56)
GGGGSGGGGSGGGGSGGGGS (SEQ ID NO: 57)
GGGGSGGGGSGGGGS (SEQ ID NO: 58)
GGGGSGGGGS (SEQ ID NO: 59)
GGGGS (SEQ ID NO: 60)
GGGSGGGS (SEQ ID NO: 61)
GGGS (SEQ ID NO: 62)
GGSGGGSGGSG (SEQ ID NO: 63)
GGSGGGSGSG (SEQ ID NO: 64)
GGSGGGSG (SEQ ID NO: 65)
Linker: (EAAAK)n (SEQ ID NO: 66) wherein n is an integer including 1, 2, 3,
4, 5, 6, 7, 8, 9, or more
CDRL1 of CD3 T-cell activating epitope binding domain: SASSSVSYMN (SEQ ID
NO: 67)
CDRL2 of CD3 T-cell activating epitope binding domain: RWIYDTSKLAS (SEQ ID
NO: 68)
CDRL3 of CD3 T-cell activating epitope binding domain: QQWSSNPFT (SEQ ID
NO: 69)
CDRH1 of CD3 T-cell activating epitope binding domain: KASGYTFTRYTMH (SEQ
ID NO: 70)
CDRH2 of CD3 T-cell activating epitope binding domain: INPSRGYTNYNQKFKD (SEQ
ID NO: 71)
CDRH3 of CD3 T-cell activating epitope binding domain: YYDDHYCLDY (SEQ ID
NO: 72)
OKT3 scFv:
QVQLQQSGAELARPGASVKMSCKASGYTFTRYTMHWVKQRPGQGLEWIGYINPSRGYTNYNQKFKDKATLTTDK
SSSTAYMQLSSLTSEDSAVYYCARYYDDHYCLDYWGQGTTLTVSSSGGGGSGGGGSGGGGSQIVLTQSPAIMSA
SPGEKVTMTCSASSSVSYMNWYQQKSGTSPKRWIYDTSKLASGVPAHFRGSGSGTSYSLTISGMEAEDAATYYC
QQWSSNPFTFGSGTKLEINR (SEQ ID NO: 73)
CDRL1 of 20G6-F3 antibody: QSLVHNNGNTY (SEQ ID NO: 74)
CDRL3 of 20G6-F3, 4E7-C9, or 4B4-D7 antibody: GQGTQYPFT (SEQ ID NO: 75)
CDRH1 of 20G6-F3 antibody: GFTFTKAW (SEQ ID NO: 76),
CDRH2 of 20G6-F3 antibody: IKDKSNSYAT (SEQ ID NO: 77)
CDRH3 of 20G6-F3 antibody: RGVYYALSPFDY (SEQ ID NO: 78)
CDRL1 of 4B4-D7 antibody: QSLVHDNGNTY (SEQ ID NO: 79)
Rat APOBEC1
MSSETGPVAVDPTLRRRIEPHEFEVFFDPRELRKETCLLYEINWGGRHSIWRHTSQNTNKHVEVNFIEKFTTER
YFCPNTRCSITWFLSWSPCGECSRAITEFLSRYPHVTLFIYIARLYHHADPRNRQGLRDLISSGVTIQIMTEQE
SGYCWRNFVNYSPSNEAHWPRYPHLWVRLYVLELYCIILGLPPCLNILRRKQPQLTFFTIALQSCHYQRLPPHI
LWATGLK (SEQ ID NO: 80)
CDRH1 of 4B4-D7 or 4E7-C9 antibody: GFTFSNAW (SEQ ID NO: 81)
CDRH2 of 4B4-D7 antibody: IKARSNNYAT (SEQ ID NO: 82)
CDRH3 of 4B4-D7 antibody: RGTYYASKPFDY (SEQ ID NO: 83)
CDRL1 of 4E7-C9 antibody: QSLEHNNGNTY (SEQ ID NO: 84)
Human APOBEC-1
MTSEKGPSTGDPTLRRRIEPWEFDVFYDPRELRKEACLLYEIKWGMSRKIWRSSGKNTTNHVEVNFIKKFTSER
DFHPSMSCSITWFLSWSPCWECSQAIREFLSRHPGVTLVIYVARLFWHMDQQNRQGLRDLVNSGVTIQIMRASE
YYHCWRNFVNYPPGDEAHWPQYPPLWMMLYALELHCIILSLPPCLKISRRWQNHLTFFRLHLQNCHYQTIPPHI
LLATGLIHPSVAWR (SEQ ID NO: 85)
Petromyzon marinus pmCDA1
MTDAEYVRIHEKLDIYTFKKQFFNNKKSVSHRCYVLFELKRRGERRACFWGYAVNKPQSGTERGIHAEIFSIRK
VEEYLRDNPGQFTINWYSSWSPCADCAEKILEWYNQELRGNGHTLKIWACKLYYEKNARNQIGLWNLRDNGVGL
NVMVSEHYQCCRKIFIQSSHNQLNENRWLEKTLKRAEKRRSELSIMIQVKILHTTKSPAV (SEQ ID NO:
86)
CDRH2 of 4E7-C9 antibody: IKDKSNNYAT (SEQ ID NO: 87)
CDRH3 of 4E7-C9 antibody: RYVHYGIGYAMDA (SEQ ID NO: 88)
CDRL1 of 18F5-H10 antibody: QSLVHTNGNTY (SEQ ID NO: 89)
CDRL3 of 18F5-H10 antibody: GQGTHYPFT (SEQ ID NO: 90)
CDRH1 of 18F5-H10 antibody: GFTFTNAW (SEQ ID NO: 91)
CDRH2 of 18F5-H10 antibody: KDKSNNYAT (SEQ ID NO: 92)
CDRH3 of 18F5-H10 antibody: RYVHYRFAYALDA (SEQ ID NO: 93)
Variable heavy chain of TGN1412:
QVQLVQSGAEVKKPGASVKVSCKASGYTFTSYYIHWVRQAPGQGLEWIGCIYPGNVNTNYNEKFKDRATLTVDT
SISTAYMELSRLRSDDTAVYFCTRSHYGLDWNFDVWGQGTTVTVSS (SEQ ID NO: 94)
Variable light chain of TGN1412:
DIQMTQSPSSLSASVGDRVTITCHASQNIYVWLNWYQQKPGKAPKLLIYKASNLHTGVPSRFSGSGSGTDFTLT
ISSLQPEDFATYYCQQGQTYPYTFGGGTKVEIK (SEQ ID NO: 95)
CDRL1 of CD28 binding domain: HASQNIYVWLN (SEQ ID NO: 96)
CDRL2 of CD28 binding domain: KASNLHT (SEQ ID NO: 97)
CDRL3 of CD28 binding domain: QQGQTYPYT (SEQ ID NO: 98)
CDRH1 of CD28 binding domain: GYTFTSYYIH (SEQ ID NO: 99)
CDRH2 of CD28 binding domain: CIYPGNVNTNYNEK (SEQ ID NO: 100)
CDRH3 of CD28 binding domain: SHYGLDWNFDV (SEQ ID NO: 101)
Human APOBEC-3G
MKPHFRNTVERMYRDTFSYNFYNRPILSRRNTVWLCYEVKTKGPSRPPLDAKIFRGQVYSELKYHPEMRFFHWF
SKWRKLHRDQEYEVTWYISWSPCTKCTRDMATFLAEDPKVTLTIFVARLYYFWDPDYQEALRSLCQKRDGPRAT
MKIMNYDEFQHCWSKFVYSQRELFEPWNNLPKYYILLHIMLGEILRHSMDPPTFTFNFNNEPWVRGRHETYLCY
EVERMHNDTWVLLNQRRGFLCNQAPHKHGFLEGRHAELCFLDVIPFWKLDLDQDYRVTCFTSWSPCFSCAQEMA
KFISKNKHVSLCIFTARIYDDQGRCQEGLRTLAEAGAKISIMTYSEFKHCWDTFVDHQGCPFQPWDGLDEHSQD
LSGRLRAILQNQEN (SEQ ID NO: 102)
Human APOBEC3G D316R_D317R APOBEC3G
MKPHFRNTVERMYRDTFSYNFYNRPILSRRNTVWLCYEVKTKGPSRPPLDAKIFRGQVYSELKYHPEMRFFHWF
SKWRKLHRDQEYEVTWYISWSPCTKCTRDMATFLAEDPKVTLTIFVARLYYFWDPDYQEALRSLCQKRDGPRAT
MKIMNYDEFQHCWSKFVYSQRELFEPWNNLPKYYILLHIMLGEILRHSMDPPTFTFNFNNEPWVRGRHETYLCY
EVERMHNDTWVLLNQRRGFLCNQAPHKHGFLEGRHAELCFLDVIPFWKLDLDQDYRVTCFTSWSPCFSCAQEMA
KFISKNKHVSLCIFTARIYRRQGRCQEGLRTLAEAGAKISIMTYSEFKHCWDTFVDHQGCPFQPWDGLDEHSQD
LSGRLRAILQNQEN (SEQ ID NO: 103)
Human APOBEC3G chain A
MDPPTETENENNEPWVRGRHETYLCYEVERMHNDTWVLLNQRRGFLCNQAPHKHGFLEGRHAELCFLDVIPEWK
LDLDQDYRVTCFTSWSPCFSCAQEMAKFISKNKHVSLCIFTARIYDDQGRCQEGLRTLAEAGAKISIMTYSEFK
HCWDTFVDHQGCPFQPWDGLDEHSQDLSGRLRAILQ (SEQ ID NO: 104)
CDRH1 of CD28 binding domain: SYYIH (SEQ ID NO: 105)
CDRH2 of CD2 8 binding domain: CIYPGNVNTNYNEKFKD (SEQ ID NO: 106)
Human APOBEC3G chain A D120R_D121R
MDPPTFTFNFNNEPWVRGRHETYLCYEVERMHNDTWVLLNQRRGFLCNQAPHKHGFLEGRHAELCFLDVIPFWK
LDLDQDYRVTCFTSWSPCFSCAQEMAKFISKNKHVSLCIFTARIYRRQGRCQEGLRTLAEAGAKISIMTYSEFK
HCWDTFVDHQGCPFQPWDGLDEHSQDLSGRLRAILQ (SEQ ID NO: 107)
CDRL1 of 4-1BB binding domain: RASQSVS (SEQ ID NO: 108)
CDRL2 of 4-1BB binding domain: ASNRAT (SEQ ID NO: 109)
CDRL3 of 4-1BB binding domain: QRSNWPPALT (SEQ ID NO: 110)
CDRH1 of 4-1BB binding domain: YYWS (SEQ ID NO: 111)
CDRH3 of 4-1BB binding domain: YGPGNYDWYFDL (SEQ ID NO: 112)
CDRL1 of 4-1BB binding domain: SGDNIGDQYAH (SEQ ID NO: 113)
CDRL2 of 4-1BB binding domain: QDKNRPS (SEQ ID NO: 114)
CDRL3 of 4-1BB binding domain: ATYTGFGSLAV (SEQ ID NO: 115)
CDRH1 of 4-1BB binding domain: GYSFSTYWIS (SEQ ID NO: 116)
CDRH2 of 4-1BB binding domain: KIYPGDSYTNYSPS (SEQ ID NO: 117)
CDRH3 of 4-1BB binding domain: GYGIFDY (SEQ ID NO: 118)
CDRL1 of OKT8 antibody: RTSRSISQYLA (SEQ ID NO: 119)
CDRL2 of OKT8 antibody: SGSTLQS (SEQ ID NO: 120)
CDRL3 of OKT8 antibody: QQHNENPLT (SEQ ID NO: 121)
CDRH1 of OKT8 antibody: GFNIKD (SEQ ID NO: 122)
CDRH2 of OKT8 antibody: RIDPANDNT (SEQ ID NO: 123)
CDRH3 of OKT8 antibody: GYGYYVFDH (SEQ ID NO: 124)
Variable light chain to KIR2DL1 and KIR2DL2/3 binding domain:
EIVLTQSPVTLSLSPGERATLSCRASQSVSSYLAWYQQKPGQAPRLLIYDASNRATGIPARFSGSGSGTDFTLT
ISSLEPEDFAVYYCQQRSNWMYTFGQGTKLEIKRT (SEQ ID NO: 125)
Variable heavy chain to KIR2DL1 and KIR2DL2/3 binding domain:
QVQLVQSGAEVKKPGSSVKVSCKASGGTFSFYAISWVRQAPGQGLEWMGGFIPIFGAANYAQKFQGRVTITADE
STSTAYMELSSLRSDDTAVYYCARIPSGSYYYDYDMDVWGQGTTVTVSS (SEQ ID NO: 126)
His Tag
HHHHHH (SEQ ID NO: 127)
Flag tag
DYKDDDDK (SEQ ID NO: 128)
Xpress tag
DLYDDDDK (SEQ ID NO: 129)
Avi tag
GLNDIFEAQKIEWHE (SEQ ID NO: 130)
Calmodulin tag
KRRWKKNFIAVSAANRFKKISSSGAL (SEQ ID NO: 131)
HA tag
YPYDVPDYA (SEQ ID NO: 132)
Myc tag
EQKLISEEDL (SEQ ID NO: 133)
Softag 1
SLAELLNAGLGGS (SEQ ID NO: 134)
Softag 3
TQDPSRVG (SEQ ID NO: 135)
V5 tag
GKPIPNPLLGLDST (SEQ ID NO: 136)
HBG-113 (ABE8e gRNA target): CUUGACCAAUAGCCUUGACA (SEQ ID NO: 137)
Each gRNA is chemically modified with the groups 2′-O-methyl analogs and 3′
phosphorothioate internucleotide linkages at the first three 5′
terminal RNA residues.
HBG-17 5 (ABE8e gRNA target): AGAUAUUUGCAUUGAGAUAG (SEQ ID NO: 138)
Optionally, each gRNA is chemically modified with the groups 2′-O-methyl
analogs and 3′ phosphorothioate internucleotide linkages at the first three
5′ and 3′ terminal RNA residues.
pCMV_BE4max
ATATGCCAAGTACGCCCCCTATTGACGTCAATGACGGTAAATGGCCCGCCTGGCATTATGCCCAGTACATGACC
TTATGGGACTTTCCTACTTGGCAGTACATCTACGTATTAGTCATCGCTATTACCATGGTGATGCGGTTTTGGCA
GTACATCAATGGGCGTGGATAGCGGTTTGACTCACGGGGATTTCCAAGTCTCCACCCCATTGACGTCAATGGGA
GTTTGTTTTGGCACCAAAATCAACGGGACTTTCCAAAATGTCGTAACAACTCCGCCCCATTGACGCAAATGGGC
GGTAGGCGTGTACGGTGGGAGGTCTATATAAGCAGAGCTGGTTTAGTGAACCGTCAGATCCGCTAGAGATCCGC
GGCCGCTAATACGACTCACTATAGGGAGAGCCGCCACCATGAAACGGACAGCCGACGGAAGCGAGTTCGAGTCA
CCAAAGAAGAAGCGGAAAGTCTCCTCAGAGACTGGGCCTGTCGCCGTCGATCCAACCCTGCGCCGCCGGATTGA
ACCTCACGAGTTTGAAGTGTTCTTTGACCCCCGGGAGCTGAGAAAGGAGACATGCCTGCTGTACGAGATCAACT
GGGGAGGCAGGCACTCCATCTGGAGGCACACCTCTCAGAACACAAATAAGCACGTGGAGGTGAACTTCATCGAG
AAGTTTACCACAGAGCGGTACTTCTGCCCCAATACCAGATGTAGCATCACATGGTTTCTGAGCTGGTCCCCTTG
CGGAGAGTGTAGCAGGGCCATCACCGAGTTCCTGTCCAGATATCCACACGTGACACTGTTTATCTACATCGCCA
GGCTGTATCACCACGCAGACCCAAGGAATAGGCAGGGCCTGCGCGATCTGATCAGCTCCGGCGTGACCATCCAG
ATCATGACAGAGCAGGAGTCCGGCTACTGCTGGCGGAACTTCGTGAATTATTCTCCTAGCAACGAGGCCCACTG
GCCTAGGTACCCACACCTGTGGGTGCGCCTGTACGTGCTGGAGCTGTATTGCATCATCCTGGGCCTGCCCCCTT
GTCTGAATATCCTGCGGAGAAAGCAGCCCCAGCTGACCTTCTTTACAATCGCCCTGCAGTCTTGTCACTATCAG
AGGCTGCCACCCCACATCCTGTGGGCCACAGGCCTGAAGTCTGGAGGATCTAGCGGAGGATCCTCTGGCAGCGA
GACACCAGGAACAAGCGAGTCAGCAACACCAGAGAGCAGTGGCGGCAGCAGCGGCGGCAGCGACAAGAAGTACA
GCATCGGCCTGGCCATCGGCACCAACTCTGTGGGCTGGGCCGTGATCACCGACGAGTACAAGGTGCCCAGCAAG
AAATTCAAGGTGCTGGGCAACACCGACCGGCACAGCATCAAGAAGAACCTGATCGGAGCCCTGCTGTTCGACAG
CGGCGAAACAGCCGAGGCCACCCGGCTGAAGAGAACCGCCAGAAGAAGATACACCAGACGGAAGAACCGGATCT
GCTATCTGCAAGAGATCTTCAGCAACGAGATGGCCAAGGTGGACGACAGCTTCTTCCACAGACTGGAAGAGTCC
TTCCTGGTGGAAGAGGATAAGAAGCACGAGCGGCACCCCATCTTCGGCAACATCGTGGACGAGGTGGCCTACCA
CGAGAAGTACCCCACCATCTACCACCTGAGAAAGAAACTGGTGGACAGCACCGACAAGGCCGACCTGCGGCTGA
TCTATCTGGCCCTGGCCCACATGATCAAGTTCCGGGGCCACTTCCTGATCGAGGGCGACCTGAACCCCGACAAC
AGCGACGTGGACAAGCTGTTCATCCAGCTGGTGCAGACCTACAACCAGCTGTTCGAGGAAAACCCCATCAACGC
CAGCGGCGTGGACGCCAAGGCCATCCTGTCTGCCAGACTGAGCAAGAGCAGACGGCTGGAAAATCTGATCGCCC
AGCTGCCCGGCGAGAAGAAGAATGGCCTGTTCGGAAACCTGATTGCCCTGAGCCTGGGCCTGACCCCCAACTTC
AAGAGCAACTTCGACCTGGCCGAGGATGCCAAACTGCAGCTGAGCAAGGACACCTACGACGACGACCTGGACAA
CCTGCTGGCCCAGATCGGCGACCAGTACGCCGACCTGTTTCTGGCCGCCAAGAACCTGTCCGACGCCATCCTGC
TGAGCGACATCCTGAGAGTGAACACCGAGATCACCAAGGCCCCCCTGAGCGCCTCTATGATCAAGAGATACGAC
GAGCACCACCAGGACCTGACCCTGCTGAAAGCTCTCGTGCGGCAGCAGCTGCCTGAGAAGTACAAAGAGATTTT
CTTCGACCAGAGCAAGAACGGCTACGCCGGCTACATTGACGGCGGAGCCAGCCAGGAAGAGTTCTACAAGTTCA
TCAAGCCCATCCTGGAAAAGATGGACGGCACCGAGGAACTGCTCGTGAAGCTGAACAGAGAGGACCTGCTGCGG
AAGCAGCGGACCTTCGACAACGGCAGCATCCCCCACCAGATCCACCTGGGAGAGCTGCACGCCATTCTGCGGCG
GCAGGAAGATTTTTACCCATTCCTGAAGGACAACCGGGAAAAGATCGAGAAGATCCTGACCTTCCGCATCCCCT
ACTACGTGGGCCCTCTGGCCAGGGGAAACAGCAGATTCGCCTGGATGACCAGAAAGAGCGAGGAAACCATCACC
CCCTGGAACTTCGAGGAAGTGGTGGACAAGGGCGCTTCCGCCCAGAGCTTCATCGAGCGGATGACCAACTTCGA
TAAGAACCTGCCCAACGAGAAGGTGCTGCCCAAGCACAGCCTGCTGTACGAGTACTTCACCGTGTATAACGAGC
TGACCAAAGTGAAATACGTGACCGAGGGAATGAGAAAGCCCGCCTTCCTGAGCGGCGAGCAGAAAAAGGCCATC
GTGGACCTGCTGTTCAAGACCAACCGGAAAGTGACCGTGAAGCAGCTGAAAGAGGACTACTTCAAGAAAATCGA
GTGCTTCGACTCCGTGGAAATCTCCGGCGTGGAAGATCGGTTCAACGCCTCCCTGGGCACATACCACGATCTGC
TGAAAATTATCAAGGACAAGGACTTCCTGGACAATGAGGAAAACGAGGACATTCTGGAAGATATCGTGCTGACC
CTGACACTGTTTGAGGACAGAGAGATGATCGAGGAACGGCTGAAAACCTATGCCCACCTGTTCGACGACAAAGT
GATGAAGCAGCTGAAGCGGCGGAGATACACCGGCTGGGGCAGGCTGAGCCGGAAGCTGATCAACGGCATCCGGG
ACAAGCAGTCCGGCAAGACAATCCTGGATTTCCTGAAGTCCGACGGCTTCGCCAACAGAAACTTCATGCAGCTG
ATCCACGACGACAGCCTGACCTTTAAAGAGGACATCCAGAAAGCCCAGGTGTCCGGCCAGGGCGATAGCCTGCA
CGAGCACATTGCCAATCTGGCCGGCAGCCCCGCCATTAAGAAGGGCATCCTGCAGACAGTGAAGGTGGTGGACG
AGCTCGTGAAAGTGATGGGCCGGCACAAGCCCGAGAACATCGTGATCGAAATGGCCAGAGAGAACCAGACCACC
CAGAAGGGACAGAAGAACAGCCGCGAGAGAATGAAGCGGATCGAAGAGGGCATCAAAGAGCTGGGCAGCCAGAT
CCTGAAAGAACACCCCGTGGAAAACACCCAGCTGCAGAACGAGAAGCTGTACCTGTACTACCTGCAGAATGGGC
GGGATATGTACGTGGACCAGGAACTGGACATCAACCGGCTGTCCGACTACGATGTGGACCATATCGTGCCTCAG
AGCTTTCTGAAGGACGACTCCATCGACAACAAGGTGCTGACCAGAAGCGACAAGAACCGGGGCAAGAGCGACAA
CGTGCCCTCCGAAGAGGTCGTGAAGAAGATGAAGAACTACTGGCGGCAGCTGCTGAACGCCAAGCTGATTACCC
AGAGAAAGTTCGACAATCTGACCAAGGCCGAGAGAGGCGGCCTGAGCGAACTGGATAAGGCCGGCTTCATCAAG
AGACAGCTGGTGGAAACCCGGCAGATCACAAAGCACGTGGCACAGATCCTGGACTCCCGGATGAACACTAAGTA
CGACGAGAATGACAAGCTGATCCGGGAAGTGAAAGTGATCACCCTGAAGTCCAAGCTGGTGTCCGATTTCCGGA
AGGATTTCCAGTTTTACAAAGTGCGCGAGATCAACAACTACCACCACGCCCACGACGCCTACCTGAACGCCGTC
GTGGGAACCGCCCTGATCAAAAAGTACCCTAAGCTGGAAAGCGAGTTCGTGTACGGCGACTACAAGGTGTACGA
CGTGCGGAAGATGATCGCCAAGAGCGAGCAGGAAATCGGCAAGGCTACCGCCAAGTACTTCTTCTACAGCAACA
TCATGAACTTTTTCAAGACCGAGATTACCCTGGCCAACGGCGAGATCCGGAAGCGGCCTCTGATCGAGACAAAC
GGCGAAACCGGGGAGATCGTGTGGGATAAGGGCCGGGATTTTGCCACCGTGCGGAAAGTGCTGAGCATGCCCCA
AGTGAATATCGTGAAAAAGACCGAGGTGCAGACAGGCGGCTTCAGCAAAGAGTCTATCCTGCCCAAGAGGAACA
GCGATAAGCTGATCGCCAGAAAGAAGGACTGGGACCCTAAGAAGTACGGCGGCTTCGACAGCCCCACCGTGGCC
TATTCTGTGCTGGTGGTGGCCAAAGTGGAAAAGGGCAAGTCCAAGAAACTGAAGAGTGTGAAAGAGCTGCTGGG
GATCACCATCATGGAAAGAAGCAGCTTCGAGAAGAATCCCATCGACTTTCTGGAAGCCAAGGGCTACAAAGAAG
TGAAAAAGGACCTGATCATCAAGCTGCCTAAGTACTCCCTGTTCGAGCTGGAAAACGGCCGGAAGAGAATGCTG
GCCTCTGCCGGCGAACTGCAGAAGGGAAACGAACTGGCCCTGCCCTCCAAATATGTGAACTTCCTGTACCTGGC
CAGCCACTATGAGAAGCTGAAGGGCTCCCCCGAGGATAATGAGCAGAAACAGCTGTTTGTGGAACAGCACAAGC
ACTACCTGGACGAGATCATCGAGCAGATCAGCGAGTTCTCCAAGAGAGTGATCCTGGCCGACGCTAATCTGGAC
AAAGTGCTGTCCGCCTACAACAAGCACCGGGATAAGCCCATCAGAGAGCAGGCCGAGAATATCATCCACCTGTT
TACCCTGACCAATCTGGGAGCCCCTGCCGCCTTCAAGTACTTTGACACCACCATCGACCGGAAGAGGTACACCA
GCACCAAAGAGGTGCTGGACGCCACCCTGATCCACCAGAGCATCACCGGCCTGTACGAGACACGGATCGACCTG
TCTCAGCTGGGAGGTGACAGCGGCGGGAGCGGCGGGAGCGGGGGGAGCACTAATCTGAGCGACATCATTGAGAA
GGAGACTGGGAAACAGCTGGTCATTCAGGAGTCCATCCTGATGCTGCCTGAGGAGGTGGAGGAAGTGATCGGCA
ACAAGCCAGAGTCTGACATCCTGGTGCACACCGCCTACGACGAGTCCACAGATGAGAATGTGATGCTGCTGACC
TCTGACGCCCCCGAGTATAAGCCTTGGGCCCTGGTCATCCAGGATTCTAACGGCGAGAATAAGATCAAGATGCT
GAGCGGAGGATCCGGAGGATCTGGAGGCAGCACCAACCTGTCTGACATCATCGAGAAGGAGACAGGCAAGCAGC
TGGTCATCCAGGAGAGCATCCTGATGCTGCCCGAAGAAGTCGAAGAAGTGATCGGAAACAAGCCTGAGAGCGAT
ATCCTGGTCCATACCGCCTACGACGAGAGTACCGACGAAAATGTGATGCTGCTGACATCCGACGCCCCAGAGTA
TAAGCCCTGGGCTCTGGTCATCCAGGATTCCAACGGAGAGAACAAAATCAAAATGCTGTCTGGCGGCTCAAAAA
GAACCGCCGACGGCAGCGAATTCGAGCCCAAGAAGAAGAGGAAAGTCTAACCGGTCATCATCACCATCACCATT
GAGTTTAAACCCGCTGATCAGCCTCGACTGTGCCTTCTAGTTGCCAGCCATCTGTTGTTTGCCCCTCCCCCGTG
CCTTCCTTGACCCTGGAAGGTGCCACTCCCACTGTCCTTTCCTAATAAAATGAGGAAATTGCATCGCATTGTCT
GAGTAGGTGTCATTCTATTCTGGGGGGTGGGGTGGGGCAGGACAGCAAGGGGGAGGATTGGGAAGACAATAGCA
GGCATGCTGGGGATGCGGTGGGCTCTATGGCTTCTGAGGCGGAAAGAACCAGCTGGGGCTCGATACCGTCGACC
TCTAGCTAGAGCTTGGCGTAATCATGGTCATAGCTGTTTCCTGTGTGAAATTGTTATCCGCTCACAATTCCACA
CAACATACGAGCCGGAAGCATAAAGTGTAAAGCCTAGGGTGCCTAATGAGTGAGCTAACTCACATTAATTGCGT
TGCGCTCACTGCCCGCTTTCCAGTCGGGAAACCTGTCGTGCCAGCTGCATTAATGAATCGGCCAACGCGCGGGG
AGAGGCGGTTTGCGTATTGGGCGCTCTTCCGCTTCCTCGCTCACTGACTCGCTGCGCTCGGTCGTTCGGCTGCG
GCGAGCGGTATCAGCTCACTCAAAGGCGGTAATACGGTTATCCACAGAATCAGGGGATAACGCAGGAAAGAACA
TGTGAGCAAAAGGCCAGCAAAAGGCCAGGAACCGTAAAAAGGCCGCGTTGCTGGCGTTTTTCCATAGGCTCCGC
CCCCCTGACGAGCATCACAAAAATCGACGCTCAAGTCAGAGGTGGCGAAACCCGACAGGACTATAAAGATACCA
GGCGTTTCCCCCTGGAAGCTCCCTCGTGCGCTCTCCTGTTCCGACCCTGCCGCTTACCGGATACCTGTCCGCCT
TTCTCCCTTCGGGAAGCGTGGCGCTTTCTCATAGCTCACGCTGTAGGTATCTCAGTTCGGTGTAGGTCGTTCGC
TCCAAGCTGGGCTGTGTGCACGAACCCCCCGTTCAGCCCGACCGCTGCGCCTTATCCGGTAACTATCGTCTTGA
GTCCAACCCGGTAAGACACGACTTATCGCCACTGGCAGCAGCCACTGGTAACAGGATTAGCAGAGCGAGGTATG
TAGGCGGTGCTACAGAGTTCTTGAAGTGGTGGCCTAACTACGGCTACACTAGAAGAACAGTATTTGGTATCTGC
GCTCTGCTGAAGCCAGTTACCTTCGGAAAAAGAGTTGGTAGCTCTTGATCCGGCAAACAAACCACCGCTGGTAG
CGGTGGTTTTTTTGTTTGCAAGCAGCAGATTACGCGCAGAAAAAAAGGATCTCAAGAAGATCCTTTGATCTTTT
CTACGGGGTCTGACACTCAGTGGAACGAAAACTCACGTTAAGGGATTTTGGTCATGAGATTATCAAAAAGGATC
TTCACCTAGATCCTTTTAAATTAAAAATGAAGTTTTAAATCAATCTAAAGTATATATGAGTAAACTTGGTCTGA
CAGTTACCAATGCTTAATCAGTGAGGCACCTATCTCAGCGATCTGTCTATTTCGTTCATCCATAGTTGCCTGAC
TCCCCGTCGTGTAGATAACTACGATACGGGAGGGCTTACCATCTGGCCCCAGTGCTGCAATGATACCGCGAGAC
CCACGCTCACCGGCTCCAGATTTATCAGCAATAAACCAGCCAGCCGGAAGGGCCGAGCGCAGAAGTGGTCCTGC
AACTTTATCCGCCTCCATCCAGTCTATTAATTGTTGCCGGGAAGCTAGAGTAAGTAGTTCGCCAGTTAATAGTT
TGCGCAACGTTGTTGCCATTGCTACAGGCATCGTGGTGTCACGCTCGTCGTTTGGTATGGCTTCATTCAGCTCC
GGTTCCCAACGATCAAGGCGAGTTACATGATCCCCCATGTTGTGCAAAAAAGCGGTTAGCTCCTTCGGTCCTCC
GATCGTTGTCAGAAGTAAGTTGGCCGCAGTGTTATCACTCATGGTTATGGCAGCACTGCATAATTCTCTTACTG
TCATGCCATCCGTAAGATGCTTTTCTGTGACTGGTGAGTACTCAACCAAGTCATTCTGAGAATAGTGTATGCGG
CGACCGAGTTGCTCTTGCCCGGCGTCAATACGGGATAATACCGCGCCACATAGCAGAACTTTAAAAGTGCTCAT
CATTGGAAAACGTTCTTCGGGGCGAAAACTCTCAAGGATCTTACCGCTGTTGAGATCCAGTTCGATGTAACCCA
CTCGTGCACCCAACTGATCTTCAGCATCTTTTACTTTCACCAGCGTTTCTGGGTGAGCAAAAACAGGAAGGCAA
AATGCCGCAAAAAAGGGAATAAGGGCGACACGGAAATGTTGAATACTCATACTCTTCCTTTTTCAATATTATTG
AAGCATTTATCAGGGTTATTGTCTCATGAGCGGATACATATTTGAATGTATTTAGAAAAATAAACAAATAGGGG
TTCCGCGCACATTTCCCCGAAAAGTGCCACCTGACGTCGACGGATCGGGAGATCGATCTCCCGATCCCCTAGGG
TCGACTCTCAGTACAATCTGCTCTGATGCCGCATAGTTAAGCCAGTATCTGCTCCCTGCTTGTGTGTTGGAGGT
CGCTGAGTAGTGCGCGAGCAAAATTTAAGCTACAACAAGGCAAGGCTTGACCGACAATTGCATGAAGAATCTGC
TTAGGGTTAGGCGTTTTGCGCTGCTTCGCGATGTACGGGCCAGATATACGCGTTGACATTGATTATTGACTAGT
TATTAATAGTAATCAATTACGGGGTCATTAGTTCATAGCCCATATATGGAGTTCCGCGTTACATAACTTACGGT
AAATGGCCCGCCTGGCTGACCGCCCAACGACCCCCGCCCATTGACGTCAATAATGACGTATGTTCCCATAGTAA
CGCCAATAGGGACTTTCCATTGACGTCAATGGGTGGAGTATTTACGGTAAACTGCCCACTTGGCAGTACATCAA
GTGTATC (SEQ ID NO: 139)
>Homo sapiens FancA cds
ATGTCCGACTCGTGGGTCCCGAACTCCGCCTCGGGCCAGGACCCAGGGGGCCGCCGGAGGGCCTGGGCCGAGCT
GCTGGCGGGAAGGGTCAAGAGGGAAAAATATAATCCTGAAAGGGCACAGAAATTAAAGGAATCAGCTGTGCGCC
TCCTGCGAAGCCATCAGGACCTGAATGCCCTTTTGCTTGAGGTAGAAGGTCCACTGTGTAAAAAATTGTCTCTC
AGCAAAGTGATTGACTGTGACAGTTCTGAGGCCTATGCTAATCATTCTAGTTCATTTATAGGCTCTGCTTTGCA
GGATCAAGCCTCAAGGCTGGGGGTTCCCGTGGGTATTCTCTCAGCCGGGATGGTTGCCTCTAGCGTGGGACAGA
TCTGCACGGCTCCAGCGGAGACCAGTCACCCTGTGCTGCTGACTGTGGAGCAGAGAAAGAAGCTGTCTTCCCTG
TTAGAGTTTGCTCAGTATTTATTGGCACACAGTATGTTCTCCCGTCTTTCCTTCTGTCAAGAATTATGGAAAAT
ACAGAGTTCTTTGTTGCTTGAAGCGGTGTGGCATCTTCACGTACAAGGCATTGTGAGCCTGCAAGAGCTGCTGG
AAAGCCATCCCGACATGCATGCTGTGGGATCGTGGCTCTTCAGGAATCTGTGCTGCCTTTGTGAACAGATGGAA
GCATCCTGCCAGCATGCTGACGTCGCCAGGGCCATGCTTTCTGATTTTGTTCAAATGTTTGTTTTGAGGGGATT
TCAGAAAAACTCAGATCTGAGAAGAACTGTGGAGCCTGAAAAAATGCCGCAGGTCACGGTTGATGTACTGCAGA
GAATGCTGATTTTTGCACTTGACGCTTTGGCTGCTGGAGTACAGGAGGAGTCCTCCACTCACAAGATCGTGAGG
TGCTGGTTCGGAGTGTTCAGTGGACACACGCTTGGCAGTGTAATTTCCACAGATCCTCTGAAGAGGTTCTTCAG
TCATACCCTGACTCAGATACTCACTCACAGCCCTGTGCTGAAAGCATCTGATGCTGTTCAGATGCAGAGAGAGT
GGAGCTTTGCGCGGACACACCCTCTGCTCACCTCACTGTACCGCAGGCTCTTTGTGATGCTGAGTGCAGAGGAG
TTGGTTGGCCATTTGCAAGAAGTTCTGGAAACGCAGGAGGTTCACTGGCAGAGAGTGCTCTCCTTTGTGTCTGC
CCTGGTTGTCTGCTTTCCAGAAGCGCAGCAGCTGCTTGAAGACTGGGTGGCGCGTTTGATGGCCCAGGCATTCG
AGAGCTGCCAGCTGGACAGCATGGTCACTGCGTTCCTGGTTGTGCGCCAGGCAGCACTGGAGGGCCCCTCTGCG
TTCCTGTCATATGCAGACTGGTTCAAGGCCTCCTTTGGGAGCACACGAGGCTACCATGGCTGCAGCAAGAAGGC
CCTGGTCTTCCTGTTTACGTTCTTGTCAGAACTCGTGCCTTTTGAGTCTCCCCGGTACCTGCAGGTGCACATTC
TCCACCCACCCCTGGTTCCCAGCAAGTACCGCTCCCTCCTCACAGACTACATCTCATTGGCCAAGACACGGCTG
GCCGACCTCAAGGTTTCTATAGAAAACATGGGACTCTACGAGGATTTGTCATCAGCTGGGGACATTACTGAGCC
CCACAGCCAAGCTCTTCAGGATGTTGAAAAGGCCATCATGGTGTTTGAGCATACGGGGAACATCCCAGTCACCG
TCATGGAGGCCAGCATATTCAGGAGGCCTTACTACGTGTCCCACTTCCTCCCCGCCCTGCTCACACCTCGAGTG
CTCCCCAAAGTCCCTGACTCCCGTGTGGCGTTTATAGAGTCTCTGAAGAGAGCAGATAAAATCCCCCCATCTCT
GTACTCCACCTACTGCCAGGCCTGCTCTGCTGCTGAAGAGAAGCCAGAAGATGCAGCCCTGGGAGTGAGGGCAG
AACCCAACTCTGCTGAGGAGCCCCTGGGACAGCTCACAGCTGCACTGGGAGAGCTGAGAGCCTCCATGACAGAC
CCCAGCCAGCGTGATGTTATATCGGCACAGGTGGCAGTGATTTCTGAAAGACTGAGGGCTGTCCTGGGCCACAA
TGAGGATGACAGCAGCGTTGAGATATCAAAGATTCAGCTCAGCATCAACACGCCGAGACTGGAGCCACGGGAAC
ACATTGCTGTGGACCTCCTGCTGACGTCTTTCTGTCAGAACCTGATGGCTGCCTCCAGTGTCGCTCCCCCGGAG
AGGCAGGGTCCCTGGGCTGCCCTCTTCGTGAGGACCATGTGTGGACGTGTGCTCCCTGCAGTGCTCACCCGGCT
CTGCCAGCTGCTCCGTCACCAGGGCCCGAGCCTGAGTGCCCCACATGTGCTGGGGTTGGCTGCCCTGGCCGTGC
ACCTGGGTGAGTCCAGGTCTGCGCTCCCAGAGGTGGATGTGGGTCCTCCTGCACCTGGTGCTGGCCTTCCTGTC
CCTGCGCTCTTTGACAGCCTCCTGACCTGTAGGACGAGGGATTCCTTGTTCTTCTGCCTGAAATTTTGTACAGC
AGCAATTTCTTACTCTCTCTGCAAGTTTTCTTCCCAGTCACGAGATACTTTGTGCAGCTGCTTATCTCCAGGCC
TTATTAAAAAGTTTCAGTTCCTCATGTTCAGATTGTTCTCAGAGGCCCGACAGCCTCTTTCTGAGGAGGACGTA
GCCAGCCTTTCCTGGAGACCCTTGCACCTTCCTTCTGCAGACTGGCAGAGAGCTGCCCTCTCTCTCTGGACACA
CAGAACCTTCCGAGAGGTGTTGAAAGAGGAAGATGTTCACTTAACTTACCAAGACTGGTTACACCTGGAGCTGG
AAATTCAACCTGAAGCTGATGCTCTTTCAGATACTGAACGGCAGGACTTCCACCAGTGGGCGATCCATGAGCAC
TTTCTCCCTGAGTCCTCGGCTTCAGGGGGCTGTGACGGAGACCTGCAGGCTGCGTGTACCATTCTTGTCAACGC
ACTGATGGATTTCCACCAAAGCTCAAGGAGTTATGACCACTCAGAAAATTCTGATTTGGTCTTTGGTGGCCGCA
CAGGAAATGAGGATATTATTTCCAGATTGCAGGAGATGGTAGCTGACCTGGAGCTGCAGCAAGACCTCATAGTG
CCTCTCGGCCACACCCCTTCCCAGGAGCACTTCCTCTTTGAGATTTTCCGCAGACGGCTCCAGGCTCTGACAAG
CGGGTGGAGCGTGGCTGCCAGCCTTCAGAGACAGAGGGAGCTGCTAATGTACAAACGGATCCTCCTCCGCCTGC
CTTCGTCTGTCCTCTGCGGCAGCAGCTTCCAGGCAGAACAGCCCATCACTGCCAGATGCGAGCAGTTCTTCCAC
TTGGTCAACTCTGAGATGAGAAACTTCTGCTCCCACGGAGGTGCCCTGACACAGGACATCACTGCCCACTTCTT
CAGGGGCCTCCTGAACGCCTGTCTGCGGAGCAGAGACCCCTCCCTGATGGTCGACTTCATACTGGCCAAGTGCC
AGACGAAATGCCCCTTAATTTTGACCTCTGCTCTGGTGTGGTGGCCGAGCCTGGAGCCTGTGCTGCTCTGCCGG
TGGAGGAGACACTGCCAGAGCCCGCTGCCCCGGGAACTGCAGAAGCTACAAGAAGGCCGGCAGTTTGCCAGCGA
TTTCCTCTCCCCTGAGGCTGCCTCCCCAGCACCCAACCCGGACTGGCTCTCAGCTGCTGCACTGCACTTTGCGA
TTCAACAAGTCAGGGAAGAAAACATCAGGAAGCAGCTAAAGAAGCTGGACTGCGAGAGAGAGGAGCTATTGGTT
TTCCTTTTCTTCTTCTCCTTGATGGGCCTGCTGTCGTCACATCTGACCTCAAATAGCACCACAGACCTGCCAAA
GGCTTTCCACGTTTGTGCAGCAATCCTCGAGTGTTTAGAGAAGAGGAAGATATCCTGGCTGGCACTCTTTCAGT
TGACAGAGAGTGACCTCAGGCTGGGGCGGCTCCTCCTCCGTGTGGCCCCGGATCAGCACACCAGGCTGCTGCCT
TTCGCTTTTTACAGTCTTCTCTCCTACTTCCATGAAGACGCGGCCATCAGGGAAGAGGCCTTCCTGCATGTTGC
TGTGGACATGTACTTGAAGCTGGTCCAGCTCTTCGTGGCTGGGGATACAAGCACAGTTTCACCTCCAGCTGGCA
GGAGCCTGGAGCTCAAGGGTCAGGGCAACCCCGTGGAACTGATAACAAAAGCTCGTCTTTTTCTGCTGCAGTTA
ATACCTCGGTGCCCGAAAAAGAGCTTCTCACACGTGGCAGAGCTGCTGGCTGATCGTGGGGACTGCGACCCAGA
GGTGAGCGCCGCCCTCCAGAGCAGACAGCAGGCTGCCCCTGACGCTGACCTGTCCCAGGAGCCTCATCTCTTCT
GA (SEQ ID NO: 140)
>Homo sapiens FANCC cds (nucleotides 263-1939 of NCBI Reference Sequence
NM_000136.2)
ATGGCTCAAGATTCAGTAGATCTTTCTTGTGATTATCAGTTTTGGATGCAGAAGCTTTCTGTATGGGATCAGGC
TTCCACTTTGGAAACCCAGCAAGACACCTGTCTTCACGTGGCTCAGTTCCAGGAGTTCCTAAGGAAGATGTATG
AAGCCTTGAAAGAGATGGATTCTAATACAGTCATTGAAAGATTCCCCACAATTGGTCAACTGTTGGCAAAAGCT
TGTTGGAATCCTTTTATTTTAGCATATGATGAAAGCCAAAAAATTCTAATATGGTGCTTATGTTGTCTAATTAA
CAAAGAACCACAGAATTCTGGACAATCAAAACTTAACTCCTGGATACAGGGTGTATTATCTCATATACTTTCAG
CACTCAGATTTGATAAAGAAGTTGCTCTTTTCACTCAAGGTCTTGGGTATGCACCTATAGATTACTATCCTGGT
TTGCTTAAAAATATGGTTTTATCATTAGCGTCTGAACTCAGAGAGAATCATCTTAATGGATTTAACACTCAAAG
GCGAATGGCTCCCGAGCGAGTGGCGTCCCTGTCACGAGTTTGTGTCCCACTTATTACCCTGACAGATGTTGACC
CCCTGGTGGAGGCTCTCCTCATCTGTCATGGACGTGAACCTCAGGAAATCCTCCAGCCAGAGTTCTTTGAGGCT
GTAAACGAGGCCATTTTGCTGAAGAAGATTTCTCTCCCCATGTCAGCTGTAGTCTGCCTCTGGCTTCGGCACCT
TCCCAGCCTTGAAAAAGCAATGCTGCATCTTTTTGAAAAGCTAATCTCCAGTGAGAGAAATTGTCTGAGAAGGA
TCGAATGCTTTATAAAAGATTCATCGCTGCCTCAAGCAGCCTGCCACCCTGCCATATTCCGGGTTGTTGATGAG
ATGTTCAGGTGTGCACTCCTGGAAACCGATGGGGCCCTGGAAATCATAGCCACTATTCAGGTGTTTACGCAGTG
CTTTGTAGAAGCTCTGGAGAAAGCAAGCAAGCAGCTGCGGTTTGCACTCAAGACCTACTTTCCTTACACTTCTC
CATCTCTTGCCATGGTGCTGCTGCAAGACCCTCAAGATATCCCTCGGGGACACTGGCTCCAGACACTGAAGCAT
ATTTCTGAACTGCTCAGAGAAGCAGTTGAAGACCAGACTCATGGGTCCTGCGGAGGTCCCTTTGAGAGCTGGTT
CCTGTTCATTCACTTCGGAGGATGGGCTGAGATGGTGGCAGAGCAATTACTGATGTCGGCAGCCGAACCCCCCA
CGGCCCTGCTGTGGCTCTTGGCCTTCTACTACGGCCCCCGTGATGGGAGGCAGCAGAGAGCACAGACTATGGTC
CAGGTGAAGGCCGTGCTGGGCCACCTCCTGGCAATGTCCAGAAGCAGCAGCCTCTCAGCCCAGGACCTGCAGAC
GGTAGCAGGACAGGGCACAGACACAGACCTCAGAGCTCCTGCACAACAGCTGATCAGGCACCTTCTCCTCAACT
TCCTGCTCTGGGCTCCTGGAGGCCACACGATCGCCTGGGATGTCATCACCCTGATGGCTCACACTGCTGAGATA
ACTCACGAGATCATTGGCTTTCTTGACCAGACCTTGTACAGATGGAATCGTCTTGGCATTGAAAGCCCTAGATC
AGAAAAACTGGCCCGAGAGCTCCTTAAAGAGCTGCGAACTCAAGTCTAG (SEQ ID NO: 141)
>Homo sapiens FANCE cds (nucleotides 186-1796 of NCBI Reference Sequence
NM_021922.2)
ATGGCGACACCGGACGCGGGGCTCCCTGGGGCTGAGGGCGTGGAGCCGGCGCCCTGGGCGCAGCTGGAGGCCCC
CGCCCGCCTCCTGCTGCAGGCGCTGCAGGCGGGGCCTGAGGGGGCGCGGCGCGGCCTGGGGGTGCTCCGGGCGC
TGGGCAGCCGCGGCTGGGAGCCCTTCGACTGGGGTCGCTTGCTCGAGGCCCTGTGCCGGGAGGAGCCGGTCGTG
CAGGGGCCTGACGGCCGTCTGGAGCTGAAACCACTGTTGCTGCGATTGCCCCGGATATGCCAGAGGAACCTGAT
GTCCCTGCTGATGGCCGTTCGGCCATCGCTGCCGGAAAGTGGGCTCCTCTCTGTGCTGCAGATTGCCCAGCAGG
ACCTAGCCCCTGACCCAGATGCCTGGCTCCGTGCCCTGGGGGAATTGCTGCGAAGGGATTTGGGGGTGGGGACC
TCCATGGAGGGAGCTTCTCCACTGTCTGAAAGATGCCAGAGACAGCTCCAAAGTCTATGTAGGGGGCTGGGCCT
GGGGGGCAGGAGGTTGAAATCCCCCCAGGCTCCAGACCCTGAAGAAGAGGAGAACAGGGACTCCCAGCAGCCTG
GGAAACGCAGAAAGGACTCAGAGGAAGAGGCTGCCAGTCCTGAGGGGAAGAGGGTCCCCAAAAGATTACGGTGT
TGGGAAGAGGAAGAAGATCATGAGAAGGAGAGACCCGAACATAAGTCACTGGAATCCCTGGCAGATGGAGGAAG
TGCATCTCCTATTAAGGACCAGCCTGTCATGGCAGTTAAGACTGGCGAGGACGGTTCGAATCTGGATGATGCTA
AAGGTCTGGCTGAGAGTTTGGAGTTGCCCAAAGCTATCCAGGACCAGCTTCCCAGGCTGCAGCAGCTGCTGAAG
ACCTTGGAGGAGGGGTTAGAGGGATTGGAGGATGCCCCCCCAGTTGAGCTACAGCTTCTTCACGAATGTAGTCC
CAGCCAGATGGACTTGCTGTGTGCCCAGCTGCAGCTCCCTCAGCTCTCAGACCTCGGTCTCCTGCGGCTCTGCA
CCTGGCTGCTGGCCCTTTCACCTGATCTCAGCCTCAGCAATGCTACTGTGCTGACCAGAAGCCTCTTTCTTGGA
CGGATCCTCTCCTTGACTTCCTCAGCCTCCCGCCTGCTTACAACTGCCCTGACCTCCTTCTGTGCCAAATATAC
ATACCCTGTCTGCAGCGCCCTCCTTGACCCTGTGCTCCAGGCCCCAGGCACAGGTCCTGCTCAAACAGAGTTAC
TGTGTTGCCTTGTGAAGATGGAGTCCCTGGAGCCAGATGCACAGGTTCTAATGCTGGGACAGATCTTGGAGCTG
CCCTGGAAGGAGGAAACTTTCTTGGTGTTGCAGTCACTCCTAGAGCGGCAGGTGGAGATGACCCCTGAGAAGTT
CAGTGTCTTAATGGAGAAGCTCTGTAAAAAGGGGCTGGCAGCCACCACCTCCATGGCCTATGCCAAGCTCATGC
TGACAGTGATGACCAAGTATCAGGCTAACATCACTGAGACCCAGAGGCTGGGCCTGGCTATGGCCCTAGAACCT
AACACCACCTTCCTGAGGAAGTCCCTGAAGGCCGCCTTGAAACATTTGGGCCCCTGA (SEQ ID NO: 142)
>Homo sapiens FANCF cds (nucleotides 32-1156 of NCBI Reference Sequence
NM_022725.3)
ATGGAATCCCTTCTGCAGCACCTGGATCGCTTTTCCGAGCTTCTGGCGGTCTCAAGCACTACCTACGTCAGCAC
CTGGGACCCCGCCACCGTGCGCCGGGCCTTGCAGTGGGCGCGCTACCTGCGCCACATCCATCGGCGCTTTGGTC
GGCATGGCCCCATTCGCACGGCTCTGGAGCGGCGGCTGCACAACCAGTGGAGGCAAGAGGGCGGCTTTGGGCGG
GGTCCAGTTCCGGGATTAGCGAACTTCCAGGCCCTCGGTCACTGTGACGTCCTGCTCTCTCTGCGCCTGCTGGA
GAACCGGGCCCTCGGGGATGCAGCTCGTTACCACCTGGTGCAGCAACTCTTTCCCGGCCCGGGCGTCCGGGACG
CCGATGAGGAGACACTCCAAGAGAGCCTGGCCCGCCTTGCCCGCCGGCGGTCTGCGGTGCACATGCTGCGCTTC
AATGGCTATAGAGAGAACCCAAATCTCCAGGAGGACTCTCTGATGAAGACCCAGGCGGAGCTGCTGCTGGAGCG
TCTGCAGGAGGTGGGGAAGGCCGAAGCGGAGCGTCCCGCCAGGTTTCTCAGCAGCCTGTGGGAGCGCTTGCCTC
AGAACAACTTCCTGAAGGTGATAGCGGTGGCGCTGTTGCAGCCGCCTTTGTCTCGTCGGCCCCAAGAAGAGTTG
GAACCCGGCATCCACAAATCACCTGGAGAGGGGAGCCAAGTGCTAGTCCACTGGCTTCTGGGGAATTCGGAAGT
CTTTGCTGCCTTTTGTCGCGCCCTCCCAGCCGGGCTTTTGACTTTAGTGACTAGCCGCCACCCAGCGCTGTCTC
CTGTCTATCTGGGTCTGCTAACAGACTGGGGTCAACGTTTGCACTATGACCTTCAGAAAGGCATTTGGGTTGGA
ACTGAGTCCCAAGATGTGCCCTGGGAGGAGTTGCACAATAGGTTTCAAAGCCTCTGTCAGGCCCCTCCACCTCT
GAAAGATAAAGTTCTAACTGCCCTGGAGACCTGTAAAGCGCAGGATGGAGATTTTGAAGTACCTGGTCTTAGCA
TCTGGACAGACCTCTTATTAGCTCTTCGTAGTGGTGCATTTAGGAAAAGACAAGTTTTGGGTCTCAGCGCAGGC
CTCAGTTCTGTATAG (SEQ ID NO: 143)
>Homo sapiens FANCG cds (nucleotides 493-2361 of NCBI Reference Sequence
NM_004629.1)
ATGTCCCGCCAGACCACCTCTGTGGGCTCCAGCTGCCTGGACCTGTGGAGGGAAAAGAATGACCGGCTCGTTCG
ACAGGCCAAGGTGGCTCAGAACTCCGGTCTGACTCTGAGGCGACAGCAGTTGGCTCAGGATGCACTGGAAGGGC
TCAGAGGGCTCCTCCATAGTCTGCAAGGGCTCCCTGCAGCTGTTCCTGTTCTTCCCTTGGAGCTGACTGTCACC
TGCAACTTCATTATCCTGAGGGCAAGCTTGGCCCAGGGTTTCACAGAGGATCAGGCCCAGGATATCCAGCGGAG
CCTAGAGAGAGTGCTGGAGACACAGGAGCAGCAGGGGCCCAGGTTGGAACAGGGGCTCAGGGAGCTGTGGGACT
CTGTCCTTCGTGCTTCCTGCCTTCTGCCGGAGCTGCTGTCTGCCCTGCACCGCCTGGTTGGCCTGCAGGCTGCC
CTCTGGTTGAGTGCTGACCGTCTTGGGGACCTGGCCTTGTTACTAGAGACCCTGAATGGCAGCCAGAGTGGAGC
CTCTAAGGATCTGCTGTTACTTCTGAAAACTTGGAGTCCCCCAGCTGAGGAATTAGATGCTCCATTGACCCTGC
AGGATGCCCAGGGATTGAAGGATGTCCTCCTGACAGCATTTGCCTACCGCCAAGGTCTCCAGGAGCTGATCACA
GGGAACCCAGACAAGGCACTAAGCAGCCTTCATGAAGCGGCCTCAGGCCTGTGTCCACGGCCTGTGTTGGTCCA
GGTGTACACAGCACTGGGGTCCTGTCACCGTAAGATGGGAAATCCACAGAGAGCACTGTTGTACTTGGTTGCAG
CCCTGAAAGAGGGATCAGCCTGGGGTCCTCCACTTCTGGAGGCCTCTAGGCTCTATCAGCAACTGGGGGACACA
ACAGCAGAGCTGGAGAGTCTGGAGCTGCTAGTTGAGGCCTTGAATGTCCCATGCAGTTCCAAAGCCCCGCAGTT
TCTCATTGAGGTAGAATTACTACTGCCACCACCTGACCTAGCCTCACCCCTTCATTGTGGCACTCAGAGCCAGA
CCAAGCACATACTAGCAAGCAGGTGCCTACAGACGGGGAGGGCAGGAGACGCTGCAGAGCATTACTTGGACCTG
CTGGCCCTGTTGCTGGATAGCTCGGAGCCAAGGTTCTCCCCACCCCCCTCCCCTCCAGGGCCCTGTATGCCTGA
GGTGTTTTTGGAGGCAGCGGTAGCACTGATCCAGGCAGGCAGAGCCCAAGATGCCTTGACTCTATGTGAGGAGT
TGCTCAGCCGCACATCATCTCTGCTACCCAAGATGTCCCGGCTGTGGGAAGATGCCAGAAAAGGAACCAAGGAA
CTGCCATACTGCCCACTCTGGGTCTCTGCCACCCACCTGCTTCAGGGCCAGGCCTGGGTTCAACTGGGTGCCCA
AAAAGTGGCAATTAGTGAATTTAGCAGGTGCCTCGAGCTGCTCTTCCGGGCCACACCTGAGGAAAAAGAACAAG
GGGCAGCTTTCAACTGTGAGCAGGGATGTAAGTCAGATGCGGCACTGCAGCAGCTTCGGGCAGCCGCCCTAATT
AGTCGTGGACTGGAATGGGTAGCCAGCGGCCAGGATACCAAAGCCTTACAGGACTTCCTCCTCAGTGTGCAGAT
GTGCCCAGGTAATCGAGACACTTACTTTCACCTGCTTCAGACTCTGAAGAGGCTAGATCGGAGGGATGAGGCCA
CTGCACTCTGGTGGAGGCTGGAGGCCCAAACTAAGGGGTCACATGAAGATGCTCTGTGGTCTCTCCCCCTGTAC
CTAGAAAGCTATTTGAGCTGGATCCGTCCCTCTGATCGTGACGCCTTCCTTGAAGAATTTCGGACATCTCTGCC
AAAGTCTTGTGACCTGTAG (SEQ ID NO: 144)
>FancA protein
MSDSWVPNSASGQDPGGRRRAWAELLAGRVKREKYNPERAQKLKESAVRLLRSHQDLNALLLEVEGPLCKKLSL
SKVIDCDSSEAYANHSSSFIGSALQDQASRLGVPVGILSAGMVASSVGQICTAPAETSHPVLLTVEQRKKLSSL
LEFAQYLLAHSMFSRLSFCQELWKIQSSLLLEAVWHLHVQGIVSLQELLESHPDMHAVGSWLFRNLCCLCEQME
ASCQHADVARAMLSDFVQMFVLRGFQKNSDLRRTVEPEKMPQVTVDVLQRMLIFALDALAAGVQEESSTHKIVR
CWFGVFSGHTLGSVISTDPLKRFFSHTLTQILTHSPVLKASDAVQMQREWSFARTHPLLTSLYRRLFVMLSAEE
LVGHLQEVLETQEVHWQRVLSFVSALWCFPEAQQLLEDWVARLMAQAFESCQLDSMVTAFLVVRQAALEGPSA
FLSYADWFKASFGSTRGYHGCSKKALVFLFTFLSELVPFESPRYLQVHILHPPLVPSKYRSLLTDYISLAKTRL
ADLKVSIENMGLYEDLSSAGDITEPHSQALQDVEKAIMVFEHTGNIPVTVMEASIFRRPYYVSHFLPALLTPRV
LPKVPDSRVAFIESLKRADKIPPSLYSTYCQACSAAEEKPEDAALGVRAEPNSAEEPLGQLTAALGELRASMTD
PSQRDVISAQVAVISERLRAVLGHNEDDSSVEISKIQLSINTPRLEPREHIAVDLLLTSFCQNLMAASSVAPPE
RQGPWAALFVRTMCGRVLPAVLTRLCQLLRHQGPSLSAPHVLGLAALAVHLGESRSALPEVDVGPPAPGAGLPV
PALFDSLLTCRTRDSLFFCLKFCTAAISYSLCKFSSQSRDTLCSCLSPGLIKKFQFLMFRLFSEARQPLSEEDV
ASLSWRPLHLPSADWQRAALSLWTHRTFREVLKEEDVHLTYQDWLHLELEIQPEADALSDTERQDFHQWAIHEH
FLPESSASGGCDGDLQAACTILVNALMDFHQSSRSYDHSENSDLVFGGRTGNEDIISRLQEMVADLELQQDLIV
PLGHTPSQEHFLFEIFRRRLQALTSGWSVAASLQRQRELLMYKRILLRLPSSVLCGSSFQAEQPITARCEQFFH
LVNSEMRNFCSHGGALTQDITAHFFRGLLNACLRSRDPSLMVDFILAKCQTKCPLILTSALVWWPSLEPVLLCR
WRRHCQSPLPRELQKLQEGRQFASDFLSPEAASPAPNPDWLSAAALHFAIQQVREENIRKQLKKLDCEREELLV
FLFFFSLMGLLSSHLTSNSTTDLPKAFHVCAAILECLEKRKISWLALFQLTESDLRLGRLLLRVAPDQHTRLLP
FAFYSLLSYFHEDAAIREEAFLHVAVDMYLKLVQLFVAGDTSTVSPPAGRSLELKGQGNPVELITKARLFLLQL
IPRCPKKSFSHVAELLADRGDCDPEVSAALQSRQQAAPDADLSQEPHLF (SEQ ID NO: 145)
>Homo sapiens FANCC (UniProt Accession Q00597)
MAQDSVDLSCDYQFWMQKLSVWDQASTLETQQDTCLHVAQFQEFLRKMYEALKEMDSNTVIERFPTIGQLLAKA
CWNPFILAYDESQKILIWCLCCLINKEPQNSGQSKLNSWIQGVLSHILSALRFDKEVALFTQGLGYAPIDYYPG
LLKNMVLSLASELRENHLNGFNTQRRMAPERVASLSRVCVPLITLTDVDPLVEALLICHGREPQEILQPEFFEA
VNEAILLKKISLPMSAVVCLWLRHLPSLEKAMLHLFEKLISSERNCLRRIECFIKDSSLPQAACHPAIFRVVDE
MFRCALLETDGALEIIATIQVFTQCFVEALEKASKQLRFALKTYFPYTSPSLAMVLLQDPQDIPRGHWLQTLKH
ISELLREAVEDQTHGSCGGPFESWFLFIHFGGWAEMVAEQLLMSAAEPPTALLWLLAFYYGPRDGRQQRAQTMV
QVKAVLGHLLAMSRSSSLSAQDLQTVAGQGTDTDLRAPAQQLIRHLLLNFLLWAPGGHTIAWDVITLMAHTAEI
THEIIGFLDQTLYRWNRLGIESPRSEKLARELLKELRTQV (SEQ ID NO: 146)
>Homo sapiens FANCE (UniProt Accession Q9HB96)
MATPDAGLPGAEGVEPAPWAQLEAPARLLLQALQAGPEGARRGLGVLRALGSRGWEPFDWGRLLEALCREEPVV
QGPDGRLELKPLLLRLPRICQRNLMSLLMAVRPSLPESGLLSVLQIAQQDLAPDPDAWLRALGELLRRDLGVGT
SMEGASPLSERCQRQLQSLCRGLGLGGRRLKSPQAPDPEEEENRDSQQPGKRRKDSEEEAASPEGKRVPKRLRC
WEEEEDHEKERPEHKSLESLADGGSASPIKDQPVMAVKTGEDGSNLDDAKGLAESLELPKAIQDQLPRLQQLLK
TLEEGLEGLEDAPPVELQLLHECSPSQMDLLCAQLQLPQLSDLGLLRLCTWLLALSPDLSLSNATVLTRSLFLG
RILSLTSSASRLLTTALTSFCAKYTYPVCSALLDPVLQAPGTGPAQTELLCCLVKMESLEPDAQVLMLGQILEL
PWKEETFLVLQSLLERQVEMTPEKFSVLMEKLCKKGLAATTSMAYAKLMLTVMTKYQANITETQRLGLAMALEP
NTTFLRKSLKAALKHLGP (SEQ ID NO: 147)
>Homo sapiens FANCF (UniProt Accession Q9NPI8)
MESLLQHLDRFSELLAVSSTTYVSTWDPATVRRALQWARYLRHIHRRFGRHGPIRTALERRLHNQWRQEGGFGR
GPVPGLANFQALGHCDVLLSLRLLENRALGDAARYHLVQQLFPGPGVRDADEETLQESLARLARRRSAVHMLRF
NGYRENPNLQEDSLMKTQAELLLERLQEVGKAEAERPARFLSSLWERLPQNNFLKVIAVALLQPPLSRRPQEEL
EPGIHKSPGEGSQVLVHWLLGNSEVFAAFCRALPAGLLTLVTSRHPALSPVYLGLLTDWGQRLHYDLQKGIWVG
TESQDVPWEELHNRFQSLCQAPPPLKDKVLTALETCKAQDGDFEVPGLSIWTDLLLALRSGAFRKRQVLGLSAG
LSSV (SEQ ID NO: 148)
>Homo sapiens FANCG (UniProt Accession I15287)
MSRQTTSVGSSCLDLWREKNDRLVRQAKVAQNSGLTLRRQQLAQDALEGLRGLLHSLQGLPAAVPVLPLELTVT
CNFIILRASLAQGFTEDQAQDIQRSLERVLETQEQQGPRLEQGLRELWDSVLRASCLLPELLSALHRLVGLQAA
LWLSADRLGDLALLLETLNGSQSGASKDLLLLLKTWSPPAEELDAPLTLQDAQGLKDVLLTAFAYRQGLQELIT
GNPDKALSSLHEAASGLCPRPVLVQVYTALGSCHRKMGNPQRALLYLVAALKEGSAWGPPLLEASRLYQQLGDT
TAELESLELLVEALNVPCSSKAPQFLIEVELLLPPPDLASPLHCGTQSQTKHILASRCLQTGRAGDAAEHYLDL
LALLLDSSEPRFSPPPSPPGPCMPEVFLEAAVALIQAGRAQDALTLCEELLSRTSSLLPKMSRLWEDARKGTKE
LPYCPLWVSATHLLQGQAWVQLGAQKVAISEFSRCLELLFRATPEEKEQGAAFNCEQGCKSDAALQQLRAAALI
SRGLEWVASGQDTKALQDFLLSVQMCPGNRDTYFHLLQTLKRLDRRDEATALWWRLEAQTKGSHEDALWSLPLY
LESYLSWIRPSDRDAFLEEFRTSLPKSCDL (SEQ ID NO: 149)
>codon optimized Human gammaC DNA
ATGCTGAAACCAAGCCTGCCCTTTACAAGCCTGCTGTTCCTGCAGCTGCCACTGCTGGGGGTCGGACTGAATAC
TACAATCCTGACACCAAACGGAAATGAGGACACCACAGCCGATTTCTTTCTGACTACCATGCCCACTGACAGTC
TGTCAGTGAGCACCCTGCCACTGCCCGAGGTCCAGTGCTTCGTGTTTAACGTCGAATATATGAACTGTACCTGG
AATAGCTCCTCTGAACCTCAGCCAACAAATCTGACTCTGCACTACTGGTATAAGAACTCTGACAATGATAAGGT
GCAGAAATGCTCACATTATCTGTTCAGCGAGGAAATCACCTCCGGCTGTCAGCTGCAGAAGAAAGAGATTCACC
TGTACCAGACATTTGTGGTCCAGCTGCAGGATCCCCGGGAACCTCGGAGACAGGCCACTCAGATGCTGAAGCTG
CAGAACCTGGTCATCCCATGGGCTCCCGAGAATCTGACCCTGCATAAACTGTCCGAGTCTCAGCTGGAACTGAA
CTGGAACAATAGGTTCCTGAATCACTGCCTGGAGCATCTGGTGCAGTACCGCACAGACTGGGATCACTCTTGGA
CTGAACAGAGTGTGGACTATCGACATAAGTTTAGTCTGCCTTCAGTGGATGGGCAGAAAAGGTACACATTCAGG
GTCCGCTCTCGGTTCAACCCACTGTGCGGAAGCGCCCAGCACTGGAGCGAGTGGTCCCACCCCATCCATTGGGG
GTCTAACACCAGCAAGGAGAATCCTTTCCTGTTTGCCCTGGAAGCTGTGGTCATTTCAGTGGGAAGCATGGGCC
TGATCATTAGCCTGCTGTGCGTGTACTTCTGGCTGGAGCGGACCATGCCTAGAATCCCAACACTGAAGAACCTG
GAGGACCTGGTGACAGAATATCACGGCAATTTTTCCGCTTGGTCTGGGGTCAGTAAAGGACTGGCAGAGAGCCT
GCAGCCCGATTACTCCGAGCGGCTGTGCCTGGTGTCCGAAATTCCCCCTAAAGGCGGGGCACTGGGAGAAGGCC
CTGGGGCCTCCCCCTGCAACCAGCACTCACCCTATTGGGCACCACCCTGTTACACCCTGAAACCCGAAACTTAA
(SEQ ID NO: 150)
>native Human gammaC DNA
ATGTTGAAGCCATCATTACCATTCACATCCCTCTTATTCCTGCAGCTGCCCCTGCTGGGAGTGGGGCTGAACAC
GACAATTCTGACGCCCAATGGGAATGAAGACACCACAGCTGATTTCTTCCTGACCACTATGCCCACTGACTCCC
TCAGCGTTTCCACTCTGCCCCTCCCAGAGGTTCAGTGTTTTGTGTTCAATGTCGAGTACATGAATTGCACTTGG
AACAGCAGCTCTGAGCCCCAGCCTACCAACCTCACTCTGCATTATTGGTACAAGAACTCGGATAATGATAAAGT
CCAGAAGTGCAGCCACTATCTATTCTCTGAAGAAATCACTTCTGGCTGTCAGTTGCAAAAAAAGGAGATCCACC
TCTACCAAACATTTGTTGTTCAGCTCCAGGACCCACGGGAACCCAGGAGACAGGCCACACAGATGCTAAAACTG
CAGAATCTGGTGATCCCCTGGGCTCCAGAGAACCTAACACTTCACAAACTGAGTGAATCCCAGCTAGAACTGAA
CTGGAACAACAGATTCTTGAACCACTGTTTGGAGCACTTGGTGCAGTACCGGACTGACTGGGACCACAGCTGGA
CTGAACAATCAGTGGATTATAGACATAAGTTCTCCTTGCCTAGTGTGGATGGGCAGAAACGCTACACGTTTCGT
GTTCGGAGCCGCTTTAACCCACTCTGTGGAAGTGCTCAGCATTGGAGTGAATGGAGCCACCCAATCCACTGGGG
GAGCAATACTTCAAAAGAGAATCCTTTCCTGTTTGCATTGGAAGCCGTGGTTATCTCTGTTGGCTCCATGGGAT
TGATTATCAGCCTTCTCTGTGTGTATTTCTGGCTGGAACGGACGATGCCCCGAATTCCCACCCTGAAGAACCTA
GAGGATCTTGTTACTGAATACCACGGGAACTTTTCGGCCTGGAGTGGTGTGTCTAAGGGACTGGCTGAGAGTCT
GCAGCCAGACTACAGTGAACGACTCTGCCTCGTCAGTGAGATTCCCCCAAAAGGAGGGGCCCTTGGGGAGGGGC
CTGGGGCCTCCCCATGCAACCAGCATAGCCCCTACTGGGCCCCCCCATGTTACACCCTAAAGCCTGAAACCTGA
(SEQ ID NO: 151)
native canine gammaC DNA
ATGTTGAAGCCACCATTGCCACTCAGATCCCTCTTATTCCTGCAGCTGTCTCTGCTGGGGGTGGGGCTGAACTC
CACGGTCCCCATGCCCAATGGGAATGAAGACATCACACCTGATTTCTTCCTGACCGCTACACCCTCCGAGACCC
TCAGTGTTTCCTCCCTGCCCCTCCCAGAGGTCCAGTGTTTTGTGTTCAATGTTGAGTACATGAATTGCACTTGG
AACAGCAGCTCTGAGCCCCGGCCCACCAACCTGACCCTGCACTACTGGTATAAGAACTCCAATGATGATAAAGT
CCAGGAGTGTGGCCACTACCTATTCTCTAGAGAGGTCACTGCTGGCTGTTGGTTGCAGAAGGAGGAGATCCATC
TCTACGAAACATTTGTTGTCCAGCTCCGGGACCCACGGGAACCCAGGAGGCAGTCCACACAGAAGCTAAAACTG
CAAAATCTGGTGATCCCCTGGGCTCCGGAGAACCTAACCCTTCACAACCTGAGCGAATCCCAGCTAGAACTGAG
CTGGAGCAACAGACACTTGGACCACTGTTTGGAGCATGTTGTGCAGTACCGGAGTGACTGGGACCGCAGCTGGA
CTGAACAGTCAGTGGACCACCGAAATAGCTTCTCTCTGCCTAGCGTGGATGGGCAGAAGTTCTACACGTTCCGT
GTCCGAAGCCGCTATAACCCACTCTGTGGAAGCGCTCAGCGTTGGAGTGAATGGAGCCACCCTATCCACTGGGG
GAGCAATACCTCCAAGGAGAATCCTTTGTTTGCATCGGAAGCTGTGCTTATCCCCCTTGGCTCCATGGGATTGA
TTATTAGCCTTATCTGTGTGTACTACTGGCTGGAACGGTCGATCCCCCGAATTCCTACCCTCAAGAACCTGGAG
GATCTGGTTACTGAATATCACGGGAATTTTTCGGCCTGGAGTGGAGTGTCTAAGGGACTGGCGGAGAGTCTGCA
GCCAGACTACAGTGAATGGCTCTGCCACGTCAGTGAGATTCCCCCAAAAGGAGGGGCTCCAGGGGAGGGTCCTG
GGGGCTCCCCCTGCAGCCAGCATAGCCCCTACTGGGCTCCCCCATGTTATACCCTGAAACCTGAAACTGGAGCC
CTGA (SEQ ID NO: 152)
>human gammaC AA
MLKPSLPFTSLLFLQLPLLGVGLNTTILTPNGNEDTTADFFLTTMPTDSLSVSTLPLPEVQCFVFNVEYMNCTW
NSSSEPQPTNLTLHYWYKNSDNDKVQKCSHYLFSEEITSGCQLQKKEIHLYQTFVVQLQDPREPRRQATOMLKL
QNLVIPWAPENLTLHKLSESQLELNWNNRFLNHCLEHLVQYRTDWDHSWTEQSVDYRHKFSLPSVDGQKRYTFR
VRSRFNPLCGSAQHWSEWSHPIHWGSNTSKENPFLFALEAVVISVGSMGLIISLLCVYFWLERTMPRIPTLKNL
EDLVTEYHGNFSAWSGVSKGLAESLQPDYSERLCLVSEIPPKGGALGEGPGASPCNQHSPYWAPPCYTLKPET
(SEQ ID NO: 153)
>native canine gammaC AA (91% conserved with human)
MLKPPLPLRSLLFLQLSLLGVGLNSTVPMPNGNEDITPDFFLTATPSETLSVSSLPLPEVQCFVFNVEYMNCTW
NSSSEPRPTNLTLHYWYKNSNDDKVQECGHYLFSREVTAGCWLQKEEIHLYETFVVQLRDPREPRRQSTQKLKL
QNLVIPWAPENLTLHNLSESQLELSWSNRHLDHCLEHVVQYRSDWDRSWTEQSVDHRNSFSLPSVDGQKFYTFR
VRSRYNPLCGSAQRWSEWSHPIHWGSNTSKENPLFASEAVLIPLGSMGLIISLICVYYWLERSIPRIPTLKNLE
DLVTEYHGNFSAWSGVSKGLAESLQPDYSEWLCHVSEIPPKGGAPGEGPGGSPCSQHSPYWAPPCYTLKPETGA
LIP (SEQ ID NO: 154)
>Homo sapiens JAK3 cds (nucleotides 101-3475 of NCBI Reference Sequence:
NM_000215.3)
ATGGCACCTCCAAGTGAAGAGACGCCCCTGATCCCTCAGCGTTCATGCAGCCTCTTGTCCACGGAGGCTGGTGC
CCTGCATGTGCTGCTGCCCGCTCGGGGCCCCGGGCCCCCCCAGCGCCTATCTTTCTCCTTTGGGGACCACTTGG
CTGAGGACCTGTGCGTGCAGGCTGCCAAGGCCAGCGGCATCCTGCCTGTGTACCACTCCCTCTTTGCTCTGGCC
ACGGAGGACCTGTCCTGCTGGTTCCCCCCGAGCCACATCTTCTCCGTGGAGGATGCCAGCACCCAAGTCCTGCT
GTACAGGATTCGCTTTTACTTCCCCAATTGGTTTGGGCTGGAGAAGTGCCACCGCTTCGGGCTACGCAAGGATT
TGGCCAGTGCTATCCTTGACCTGCCAGTCCTGGAGCACCTCTTTGCCCAGCACCGCAGTGACCTGGTGAGTGGG
CGCCTCCCCGTGGGCCTCAGTCTCAAGGAGCAGGGTGAGTGTCTCAGCCTGGCCGTGTTGGACCTGGCCCGGAT
GGCGCGAGAGCAGGCCCAGCGGCCGGGAGAGCTGCTGAAGACTGTCAGCTACAAGGCCTGCCTACCCCCAAGCC
TGCGCGACCTGATCCAGGGCCTGAGCTTCGTGACGCGGAGGCGTATTCGGAGGACGGTGCGCAGAGCCCTGCGC
CGCGTGGCCGCCTGCCAGGCAGACCGGCACTCGCTCATGGCCAAGTACATCATGGACCTGGAGCGGCTGGATCC
AGCCGGGGCCGCCGAGACCTTCCACGTGGGCCTCCCTGGGGCCCTTGGTGGCCACGACGGGCTGGGGCTGCTCC
GCGTGGCTGGTGACGGCGGCATCGCCTGGACCCAGGGAGAACAGGAGGTCCTCCAGCCCTTCTGCGACTTTCCA
GAAATCGTAGACATTAGCATCAAGCAGGCCCCGCGCGTTGGCCCGGCCGGAGAGCACCGCCTGGTCACTGTTAC
CAGGACAGACAACCAGATTTTAGAGGCCGAGTTCCCAGGGCTGCCCGAGGCTCTGTCGTTCGTGGCGCTCGTGG
ACGGCTACTTCCGGCTGACCACGGACTCCCAGCACTTCTTCTGCAAGGAGGTGGCACCGCCGAGGCTGCTGGAG
GAAGTGGCCGAGCAGTGCCACGGCCCCATCACTCTGGACTTTGCCATCAACAAGCTCAAGACTGGGGGCTCACG
TCCTGGCTCCTATGTTCTCCGCCGCAGCCCCCAGGACTTTGACAGCTTCCTCCTCACTGTCTGTGTCCAGAACC
CCCTTGGTCCTGATTATAAGGGCTGCCTCATCCGGCGCAGCCCCACAGGAACCTTCCTTCTGGTTGGCCTCAGC
CGACCCCACAGCAGTCTTCGAGAGCTCCTGGCAACCTGCTGGGATGGGGGGCTGCACGTAGATGGGGTGGCAGT
GACCCTCACTTCCTGCTGTATCCCCAGACCCAAAGAAAAGTCCAACCTGATCGTGGTCCAGAGAGGTCACAGCC
CACCCACATCATCCTTGGTTCAGCCCCAATCCCAATACCAGCTGAGTCAGATGACATTTCACAAGATCCCTGCT
GACAGCCTGGAGTGGCATGAGAACCTGGGCCATGGGTCCTTCACCAAGATTTACCGGGGCTGTCGCCATGAGGT
GGTGGATGGGGAGGCCCGAAAGACAGAGGTGCTGCTGAAGGTCATGGATGCCAAGCACAAGAACTGCATGGAGT
CATTCCTGGAAGCAGCGAGCTTGATGAGCCAAGTGTCGTACCGGCATCTCGTGCTGCTCCACGGCGTGTGCATG
GCTGGAGACAGCACCATGGTGCAGGAATTTGTACACCTGGGGGCCATAGACATGTATCTGCGAAAACGTGGCCA
CCTGGTGCCAGCCAGCTGGAAGCTGCAGGTGGTCAAACAGCTGGCCTACGCCCTCAACTATCTGGAGGACAAAG
GCCTGCCCCATGGCAATGTCTCTGCCCGGAAGGTGCTCCTGGCTCGGGAGGGGGCTGATGGGAGCCCGCCCTTC
ATCAAGCTGAGTGACCCTGGGGTCAGCCCCGCTGTGTTAAGCCTGGAGATGCTCACCGACAGGATCCCCTGGGT
GGCCCCCGAGTGTCTCCGGGAGGCGCAGACACTTAGCTTGGAAGCTGACAAGTGGGGCTTCGGCGCCACGGTCT
GGGAAGTGTTTAGTGGCGTCACCATGCCCATCAGTGCCCTGGATCCTGCTAAGAAACTCCAATTTTATGAGGAC
CGGCAGCAGCTGCCGGCCCCCAAGTGGACAGAGCTGGCCCTGCTGATTCAACAGTGCATGGCCTATGAGCCGGT
CCAGAGGCCCTCCTTCCGAGCCGTCATTCGTGACCTCAATAGCCTCATCTCTTCAGACTATGAGCTCCTCTCAG
ACCCCACACCTGGTGCCCTGGCACCTCGTGATGGGCTGTGGAATGGTGCCCAGCTCTATGCCTGCCAAGACCCC
ACGATCTTCGAGGAGAGACACCTCAAGTACATCTCACAGCTGGGCAAGGGCAACTTTGGCAGCGTGGAGCTGTG
CCGCTATGACCCGCTAGGCGACAATACAGGTGCCCTGGTGGCCGTGAAACAGCTGCAGCACAGCGGGCCAGACC
AGCAGAGGGACTTTCAGCGGGAGATTCAGATCCTCAAAGCACTGCACAGTGATTTCATTGTCAAGTATCGTGGT
GTCAGCTATGGCCCGGGCCGCCAGAGCCTGCGGCTGGTCATGGAGTACCTGCCCAGCGGCTGCTTGCGCGACTT
CCTGCAGCGGCACCGCGCGCGCCTCGATGCCAGCCGCCTCCTTCTCTATTCCTCGCAGATCTGCAAGGGCATGG
AGTACCTGGGCTCCCGCCGCTGCGTGCACCGCGACCTGGCCGCCCGAAACATCCTCGTGGAGAGCGAGGCACAC
GTCAAGATCGCTGACTTCGGCCTAGCTAAGCTGCTGCCGCTTGACAAAGACTACTACGTGGTCCGCGAGCCAGG
CCAGAGCCCCATTTTCTGGTATGCCCCCGAATCCCTCTCGGACAACATCTTCTCTCGCCAGTCAGACGTCTGGA
GCTTCGGGGTCGTCCTGTACGAGCTCTTCACCTACTGCGACAAAAGCTGCAGCCCCTCGGCCGAGTTCCTGCGG
ATGATGGGATGTGAGCGGGATGTCCCCGCCCTCTGCCGCCTCTTGGAACTGCTGGAGGAGGGCCAGAGGCTGCC
GGCGCCTCCTGCCTGCCCTGCTGAGGTTCACGAGCTCATGAAGCTGTGCTGGGCCCCTAGCCCACAGGACCGGC
CATCATTCAGCGCCCTGGGCCCCCAGCTGGACATGCTGTGGAGCGGAAGCCGGGGGTGTGAGACTCATGCCTTC
ACTGCTCACCCAGAGGGCAAACACCACTCCCTGTCCTTTTCATAG (SEQ ID NO: 155)
>Homo sapiens PNP cds (nucleotides 147-1016 of NCBI Reference Sequence:
NM_000270.3)
ATGGAGAACGGATACACCTATGAAGATTATAAGAACACTGCAGAATGGCTTCTGTCTCACACTAAGCACCGACC
TCAAGTTGCAATAATCTGTGGTTCTGGATTAGGAGGTCTGACTGATAAATTAACTCAGGCCCAGATCTTTGACT
ACGGTGAAATCCCCAACTTTCCCCGAAGTACAGTGCCAGGTCATGCTGGCCGACTGGTGTTTGGGTTCCTGAAT
GGCAGGGCCTGTGTGATGATGCAGGGCAGGTTCCACATGTATGAAGGGTACCCACTCTGGAAGGTGACATTCCC
AGTGAGGGTTTTCCACCTTCTGGGTGTGGACACCCTGGTAGTCACCAATGCAGCAGGAGGGCTGAACCCCAAGT
TTGAGGTTGGAGATATCATGCTGATCCGTGACCATATCAACCTACCTGGTTTCAGTGGTCAGAACCCTCTCAGA
GGGCCCAATGATGAAAGGTTTGGAGATCGTTTCCCTGCCATGTCTGATGCCTACGACCGGACTATGAGGCAGAG
GGCTCTCAGTACCTGGAAACAAATGGGGGAGCAACGTGAGCTACAGGAAGGCACCTATGTGATGGTGGCAGGCC
CCAGCTTTGAGACTGTGGCAGAATGTCGTGTGCTGCAGAAGCTGGGAGCAGACGCTGTTGGCATGAGTACAGTA
CCAGAAGTTATCGTTGCACGGCACTGTGGACTTCGAGTCTTTGGCTTCTCACTCATCACTAACAAGGTCATCAT
GGATTATGAAAGCCTGGAGAAGGCCAACCATGAAGAAGTCTTAGCAGCTGGCAAACAAGCTGCACAGAAATTGG
AACAGTTTGTCTCCATTCTTATGGCCAGCATTCCACTCCCTGACAAAGCCAGTTGA (SEQ ID NO: 156)
>Homo sapiens ADA cds (nucleotides 152-1243 of NCBI Reference Sequence:
NM_000022.3)
ATGGCCCAGACGCCCGCCTTCGACAAGCCCAAAGTAGAACTGCATGTCCACCTAGACGGATCCATCAAGCCTGA
AACCATCTTATACTATGGCAGGAGGAGAGGGATCGCCCTCCCAGCTAACACAGCAGAGGGGCTGCTGAACGTCA
TTGGCATGGACAAGCCGCTCACCCTTCCAGACTTCCTGGCCAAGTTTGACTACTACATGCCTGCTATCGCGGGC
TGCCGGGAGGCTATCAAAAGGATCGCCTATGAGTTTGTAGAGATGAAGGCCAAAGAGGGCGTGGTGTATGTGGA
GGTGCGGTACAGTCCGCACCTGCTGGCCAACTCCAAAGTGGAGCCAATCCCCTGGAACCAGGCTGAAGGGGACC
TCACCCCAGACGAGGTGGTGGCCCTAGTGGGCCAGGGCCTGCAGGAGGGGGAGCGAGACTTCGGGGTCAAGGCC
CGGTCCATCCTGTGCTGCATGCGCCACCAGCCCAACTGGTCCCCCAAGGTGGTGGAGCTGTGTAAGAAGTACCA
GCAGCAGACCGTGGTAGCCATTGACCTGGCTGGAGATGAGACCATCCCAGGAAGCAGCCTCTTGCCTGGACATG
TCCAGGCCTACCAGGAGGCTGTGAAGAGCGGCATTCACCGTACTGTCCACGCCGGGGAGGTGGGCTCGGCCGAA
GTAGTAAAAGAGGCTGTGGACATACTCAAGACAGAGCGGCTGGGACACGGCTACCACACCCTGGAAGACCAGGC
CCTTTATAACAGGCTGCGGCAGGAAAACATGCACTTCGAGATCTGCCCCTGGTCCAGCTACCTCACTGGTGCCT
GGAAGCCGGACACGGAGCATGCAGTCATTCGGCTCAAAAATGACCAGGCTAACTACTCGCTCAACACAGATGAC
CCGCTCATCTTCAAGTCCACCCTGGACACTGATTACCAGATGACCAAACGGGACATGGGCTTTACTGAAGAGGA
GTTTAAAAGGCTGAACATCAATGCGGCCAAATCTAGTTTCCTCCCAGAAGATGAAAAGAGGGAGCTTCTCGACC
TGCTCTATAAAGCCTATGGGATGCCACCTTCAGCCTCTGCAGGGCAGAACCTCTGA (SEQ ID NO: 157)
>Homo sapiens RAG1 cds (nucleotides 125-3256 of NCBI Reference Sequence:
NM_000448.2)
ATGGCAGCCTCTTTCCCACCCACCTTGGGACTCAGTTCTGCCCCAGATGAAATTCAGCACCCACATATTAAATT
TTCAGAATGGAAATTTAAGCTGTTCCGGGTGAGATCCTTTGAAAAGACACCTGAAGAAGCTCAAAAGGAAAAGA
AGGATTCCTTTGAGGGGAAACCCTCTCTGGAGCAATCTCCAGCAGTCCTGGACAAGGCTGATGGTCAGAAGCCA
GTCCCAACTCAGCCATTGTTAAAAGCCCACCCTAAGTTTTCAAAGAAATTTCACGACAACGAGAAAGCAAGAGG
CAAAGCGATCCATCAAGCCAACCTTCGACATCTCTGCCGCATCTGTGGGAATTCTTTTAGAGCTGATGAGCACA
ACAGGAGATATCCAGTCCATGGTCCTGTGGATGGTAAAACCCTAGGCCTTTTACGAAAGAAGGAAAAGAGAGCT
ACTTCCTGGCCGGACCTCATTGCCAAGGTTTTCCGGATCGATGTGAAGGCAGATGTTGACTCGATCCACCCCAC
TGAGTTCTGCCATAACTGCTGGAGCATCATGCACAGGAAGTTTAGCAGTGCCCCATGTGAGGTTTACTTCCCGA
GGAACGTGACCATGGAGTGGCACCCCCACACACCATCCTGTGACATCTGCAACACTGCCCGTCGGGGACTCAAG
AGGAAGAGTCTTCAGCCAAACTTGCAGCTCAGCAAAAAACTCAAAACTGTGCTTGACCAAGCAAGACAAGCCCG
TCAGCGCAAGAGAAGAGCTCAGGCAAGGATCAGCAGCAAGGATGTCATGAAGAAGATCGCCAACTGCAGTAAGA
TACATCTTAGTACCAAGCTCCTTGCAGTGGACTTCCCAGAGCACTTTGTGAAATCCATCTCCTGCCAGATCTGT
GAACACATTCTGGCTGACCCTGTGGAGACCAACTGTAAGCATGTCTTTTGCCGGGTCTGCATTCTCAGATGCCT
CAAAGTCATGGGCAGCTATTGTCCCTCTTGCCGATATCCATGCTTCCCTACTGACCTGGAGAGTCCAGTGAAGT
CCTTTCTGAGCGTCTTGAATTCCCTGATGGTGAAATGTCCAGCAAAAGAGTGCAATGAGGAGGTCAGTTTGGAA
AAATATAATCACCACATCTCAAGTCACAAGGAATCAAAAGAGATTTTTGTGCACATTAATAAAGGGGGCCGGCC
CCGCCAACATCTTCTGTCGCTGACTCGGAGAGCTCAGAAGCACCGGCTGAGGGAGCTCAAGCTGCAAGTCAAAG
CCTTTGCTGACAAAGAAGAAGGTGGAGATGTGAAGTCCGTGTGCATGACCTTGTTCCTGCTGGCTCTGAGGGCG
AGGAATGAGCACAGGCAAGCTGATGAGCTGGAGGCCATCATGCAGGGAAAGGGCTCTGGCCTGCAGCCAGCTGT
TTGCTTGGCCATCCGTGTCAACACCTTCCTCAGCTGCAGTCAGTACCACAAGATGTACAGGACTGTGAAAGCCA
TCACAGGGAGACAGATTTTTCAGCCTTTGCATGCCCTTCGGAATGCTGAGAAGGTACTTCTGCCAGGCTACCAC
CACTTTGAGTGGCAGCCACCTCTGAAGAATGTGTCTTCCAGCACTGATGTTGGCATTATTGATGGGCTGTCTGG
ACTATCATCCTCTGTGGATGATTACCCAGTGGACACCATTGCAAAGAGGTTCCGCTATGATTCAGCTTTGGTGT
CTGCTTTGATGGACATGGAAGAAGACATCTTGGAAGGCATGAGATCCCAAGACCTTGATGATTACCTGAATGGC
CCCTTCACTGTGGTGGTGAAGGAGTCTTGTGATGGAATGGGAGACGTGAGTGAGAAGCATGGGAGTGGGCCTGT
AGTTCCAGAAAAGGCAGTCCGTTTTTCATTCACAATCATGAAAATTACTATTGCCCACAGCTCTCAGAATGTGA
AAGTATTTGAAGAAGCCAAACCTAACTCTGAACTGTGTTGCAAGCCATTGTGCCTTATGCTGGCAGATGAGTCT
GACCACGAGACGCTGACTGCCATCCTGAGTCCTCTCATTGCTGAGAGGGAGGCCATGAAGAGCAGTGAATTAAT
GCTTGAGCTGGGAGGCATTCTCCGGACTTTCAAGTTCATCTTCAGGGGCACCGGCTATGATGAAAAACTTGTGC
GGGAAGTGGAAGGCCTCGAGGCTTCTGGCTCAGTCTACATTTGTACTCTTTGTGATGCCACCCGTCTGGAAGCC
TCTCAAAATCTTGTCTTCCACTCTATAACCAGAAGCCATGCTGAGAACCTGGAACGTTATGAGGTCTGGCGTTC
CAACCCTTACCATGAGTCTGTGGAAGAACTGCGGGATCGGGTGAAAGGGGTCTCAGCTAAACCTTTCATTGAGA
CAGTCCCTTCCATAGATGCACTCCACTGTGACATTGGCAATGCAGCTGAGTTCTACAAGATCTTCCAGCTAGAG
ATAGGGGAAGTGTATAAGAATCCCAATGCTTCCAAAGAGGAAAGGAAAAGGTGGCAGGCCACACTGGACAAGCA
TCTCCGGAAGAAGATGAACCTCAAACCAATCATGAGGATGAATGGCAACTTTGCCAGGAAGCTCATGACCAAAG
AGACTGTGGATGCAGTTTGTGAGTTAATTCCTTCCGAGGAGAGGCACGAGGCTCTGAGGGAGCTGATGGATCTT
TACCTGAAGATGAAACCAGTATGGCGATCATCATGCCCTGCTAAAGAGTGCCCAGAATCCCTCTGCCAGTACAG
TTTCAATTCACAGCGTTTTGCTGAGCTCCTTTCTACGAAGTTCAAGTATAGGTATGAGGGAAAAATCACCAATT
ATTTTCACAAAACCCTGGCCCATGTTCCTGAAATTATTGAGAGGGATGGCTCCATTGGGGCATGGGCAAGTGAG
GGAAATGAGTCTGGTAACAAACTGTTTAGGCGCTTCCGGAAAATGAATGCCAGGCAGTCCAAATGCTATGAGAT
GGAAGATGTCCTGAAACACCACTGGTTGTACACCTCCAAATACCTCCAGAAGTTTATGAATGCTCATAATGCAT
TAAAAACCTCTGGGTTTACCATGAACCCTCAGGCAAGCTTAGGGGACCCATTAGGCATAGAGGACTCTCTGGAA
AGCCAAGATTCAATGGAATTTTAA (SEQ ID NO: 158)
>Homo sapiens RAG2 cds (nucleotides 206-1789 of NCBI Reference Sequence:
NM_000536.3)
ATGTCTCTGCAGATGGTAACAGTCAGTAATAACATAGCCTTAATTCAGCCAGGCTTCTCACTGATGAATTTTGA
TGGACAAGTTTTCTTCTTTGGACAAAAAGGCTGGCCCAAAAGATCCTGCCCCACTGGAGTTTTCCATCTGGATG
TAAAGCATAACCATGTCAAACTGAAGCCTACAATTTTCTCTAAGGATTCCTGCTACCTCCCTCCTCTTCGCTAC
CCAGCCACTTGCACATTCAAAGGCAGCTTGGAGTCTGAAAAGCATCAATACATCATCCATGGAGGGAAAACACC
AAACAATGAGGTTTCAGATAAGATTTATGTCATGTCTATTGTTTGCAAGAACAACAAAAAGGTTACTTTTCGCT
GCACAGAGAAAGACTTGGTAGGAGATGTTCCTGAAGCCAGATATGGTCATTCCATTAATGTGGTGTACAGCCGA
GGGAAAAGTATGGGTGTTCTCTTTGGAGGACGCTCATACATGCCTTCTACCCACAGAACCACAGAAAAATGGAA
TAGTGTAGCTGACTGCCTGCCCTGTGTTTTCCTGGTGGATTTTGAATTTGGGTGTGCTACATCATACATTCTTC
CAGAACTTCAGGATGGGCTATCTTTTCATGTCTCTATTGCCAAAAATGACACCATCTATATTTTAGGAGGACAT
TCACTTGCCAATAATATCCGGCCTGCCAACCTGTACAGAATAAGGGTTGATCTTCCCCTGGGTAGCCCAGCTGT
GAATTGCACAGTCTTGCCAGGAGGAATCTCTGTCTCCAGTGCAATCCTGACTCAAACTAACAATGATGAATTTG
TTATTGTTGGTGGCTATCAGCTTGAAAATCAAAAAAGAATGATCTGCAACATCATCTCTTTAGAGGACAACAAG
ATAGAAATTCGTGAGATGGAGACCCCAGATTGGACCCCAGACATTAAGCACAGCAAGATATGGTTTGGAAGCAA
CATGGGAAATGGAACTGTTTTTCTTGGCATACCAGGAGACAATAAACAAGTTGTTTCAGAAGGATTCTATTTCT
ATATGTTGAAATGTGCTGAAGATGATACTAATGAAGAGCAGACAACATTCACAAACAGTCAAACATCAACAGAA
GATCCAGGGGATTCCACTCCCTTTGAAGACTCTGAAGAATTTTGTTTCAGTGCAGAAGCAAATAGTTTTGATGG
TGATGATGAATTTGACACCTATAATGAAGATGATGAAGAAGATGAGTCTGAGACAGGCTACTGGATTACATGCT
GCCCTACTTGTGATGTGGATATCAACACTTGGGTACCATTCTATTCAACTGAGCTCAACAAACCCGCCATGATC
TACTGCTCTCATGGGGATGGGCACTGGGTCCATGCTCAGTGCATGGATCTGGCAGAACGCACACTCATCCATCT
GTCAGCAGGAAGCAACAAGTATTACTGCAATGAGCATGTGGAGATAGCAAGAGCTCTACACACTCCCCAAAGAG
TCCTACCCTTAAAAAAGCCTCCAATGAAATCCCTCCGTAAAAAAGGTTCTGGAAAAATCTTGACTCCTGCCAAG
AAATCCTTTCTTAGAAGGTTGTTTGATTAG (SEQ ID NO: 159)
>Homo sapiens JAK3 isoform 2 (UniProt Accession P52333-1)
MAPPSEETPLIPQRSCSLLSTEAGALHVLLPARGPGPPQRLSFSFGDHLAEDLCVQAAKASGILPVYHSLFALA
TEDLSCWFPPSHIFSVEDASTQVLLYRIRFYFPNWFGLEKCHRFGLRKDLASAILDLPVLEHLFAQHRSDLVSG
RLPVGLSLKEQGECLSLAVLDLARMAREQAQRPGELLKTVSYKACLPPSLRDLIQGLSFVTRRRIRRTVRRALR
RVAACQADRHSLMAKYIMDLERLDPAGAAETFHVGLPGALGGHDGLGLLRVAGDGGIAWTQGEQEVLQPFCDFP
EIVDISIKQAPRVGPAGEHRLVTVTRTDNQILEAEFPGLPEALSFVALVDGYFRLTTDSQHFFCKEVAPPRLLE
EVAEQCHGPITLDFAINKLKTGGSRPGSYVLRRSPQDFDSFLLTVCVQNPLGPDYKGCLIRRSPTGTFLLVGLS
RPHSSLRELLATCWDGGLHVDGVAVTLTSCCIPRPKEKSNLIVVQRGHSPPTSSLVQPQSQYQLSQMTFHKIPA
DSLEWHENLGHGSFTKIYRGCRHEVVDGEARKTEVLLKVMDAKHKNCMESFLEAASLMSQVSYRHLVLLHGVCM
AGDSTMVQEFVHLGAIDMYLRKRGHLVPASWKLQVVKQLAYALNYLEDKGLPHGNVSARKVLLAREGADGSPPF
IKLSDPGVSPAVLSLEMLTDRIPWVAPECLREAQTLSLEADKWGFGATVWEVFSGVTMPISALDPAKKLQFYED
RQQLPAPKWTELALLIQQCMAYEPVQRPSFRAVIRDLNSLISSDYELLSDPTPGALAPRDGLWNGAQLYACQDP
TIFEERHLKYISQLGKGNFGSVELCRYDPLGDNTGALVAVKQLQHSGPDQQRDFQREIQILKALHSDFIVKYRG
VSYGPGRQSLRLVMEYLPSGCLRDFLQRHRARLDASRLLLYSSQICKGMEYLGSRRCVHRDLAARNILVESEAH
VKIADFGLAKLLPLDKDYYWREPGQSPIFVVYAPESLSDNIFSRQSDVWSFGVVLYELFTYCDKSCSPSAEFLR
MMGCERDVPALCRLLELLEEGQRLPAPPACPAEVHELMKLCWAPSPQDRPSFSALGPQLDMLWSGSRGCETHAF
TAHPEGKHHSLSFS (SEQ ID NO: 160)
>Homo sapiens PNP (UniProt Accession P00491)
MENGYTYEDYKNTAEWLLSHTKHRPQVAIICGSGLGGLTDKLTQAQIFDYGEIPNFPRSTVPGHAGRLVFGFLN
GRACVMMQGRFHMYEGYPLWKVTFPVRVFHLLGVDTLVVTNAAGGLNPKFEVGDIMLIRDHINLPGFSGQNPLR
GPNDERFGDRFPAMSDAYDRTMRQRALSTWKQMGEQRELQEGTYVMVAGPSFETVAECRVLQKLGADAVGMSTV
PEVIVARHCGLRVFGFSLITNKVIMDYESLEKANHEEVLAAGKQAAQKLEQFVSILMASIPLPDKAS (SEQ
ID NO: 161)
>Homo sapiens ADA (UniProt Accession P00813)
MAQTPAFDKPKVELHVHLDGSIKPETILYYGRRRGIALPANTAEGLLNVIGMDKPLTLPDFLAKFDYYMPAIAG
CREAIKRIAYEFVEMKAKEGVVYVEVRYSPHLLANSKVEPIPWNQAEGDLTPDEVVALVGQGLQEGERDFGVKA
RSILCCMRHQPNWSPKVVELCKKYQQQTVVAIDLAGDETIPGSSLLPGHVQAYQEAVKSGIHRTVHAGEVGSAE
VVKEAVDILKTERLGHGYHTLEDQALYNRLRQENMHFEICPWSSYLTGAWKPDTEHAVIRLKNDQANYSLNTDD
PLIFKSTLDTDYQMTKRDMGFTEEEFKRLNINAAKSSFLPEDEKRELLDLLYKAYGMPPSASAGQNL (SEQ
ID NO: 162)
>Homo sapiens RAGI isoform 1 (UniProt Accession P15918-1)
MAASFPPTLGLSSAPDEIQHPHIKFSEWKFKLFRVRSFEKTPEEAQKEKKDSFEGKPSLEQSPAVLDKADGQKP
VPTQPLLKAHPKFSKKFHDNEKARGKAIHQANLRHLCRICGNSFRADEHNRRYPVHGPVDGKTLGLLRKKEKRA
TSWPDLIAKVFRIDVKADVDSIHPTEFCHNCWSIMHRKFSSAPCEVYFPRNVTMEWHPHTPSCDICNTARRGLK
RKSLQPNLQLSKKLKTVLDQARQARQHKRRAQARISSKDVMKKIANCSKIHLSTKLLAVDFPEHEVKSISCQIC
EHILADPVETNCKHVFCRVCILRCLKVMGSYCPSCRYPCFPTDLESPVKSFLSVLNSLMVKCPAKECNEEVSLE
KYNHHISSHKESKEIFVHINKGGRPRQHLLSLTRRAQKHRLRELKLQVKAFADKEEGGDVKSVCMTLFLLALRA
RNEHRQADELEAIMQGKGSGLQPAVCLAIRVNTFLSCSQYHKMYRTVKAITGRQIFQPLHALRNAEKVLLPGYH
HFEWQPPLKNVSSSTDVGIIDGLSGLSSSVDDYPVDTIAKRFRYDSALVSALMDMEEDILEGMRSQDLDDYLNG
PFTVVVKESCDGMGDVSEKHGSGPVVPEKAVRFSFTIMKITIAHSSQNVKVFEEAKPNSELCCKPLCLMLADES
DHETLTAILSPLIAEREAMKSSELMLELGGILRTFKFIFRGTGYDEKLVREVEGLEASGSVYICTLCDATRLEA
SQNLVFHSITRSHAENLERYEVWRSNPYHESVEELRDRVKGVSAKPFIETVPSIDALHCDIGNAAEFYKIFQLE
IGEVYKNPNASKEERKRWQATLDKHLRKKMNLKPIMRMNGNFARKLMTKETVDAVCELIPSEERHEALRELMDL
YLKMKPVWRSSCPAKECPESLCQYSFNSQRFAELLSTKFKYRYEGKITNYFHKTLAHVPEIIERDGSIGAWASE
GNESGNKLFRRFRKMNARQSKCYEMEDVLKHHWLYTSKYLQKFMNAHNALKTSGFTMNPQASLGDPLGIEDSLE
SQDSMEF (SEQ ID NO: 163)
>Homo sapiens RAG2 (UniProt Accession P55895)
MSLQMVTVSNNIALIQPGFSLMNFDGQVFFFGQKGWPKRSCPTGVFHLDVKHNHVKLKPTIFSKDSCYLPPLRY
PATCTFKGSLESEKHQYIIHGGKTPNNEVSDKIYVMSIVCKNNKKVTFRCTEKDLVGDVPEARYGHSINVVYSR
GKSMGVLFGGRSYMPSTHRTTEKWNSVADCLPCVFLVDFEFGCATSYILPELQDGLSFHVSIAKNDTIYILGGH
SLANNIRPANLYRIRVDLPLGSPAVNCTVLPGGISVSSAILTQTNNDEFVIVGGYQLENQKRMICNIISLEDNK
IEIREMETPDWTPDIKHSKIWFGSNMGNGTVFLGIPGDNKQVVSEGFYFYMLKCAEDDTNEEQTTFTNSQTSTE
DPGDSTPFEDSEEFCFSAEANSFDGDDEFDTYNEDDEEDESETGYWITCCPTCDVDINTWVPFYSTELNKPAMI
YCSHGDGHWVHAQCMDLAERTLIHLSAGSNKYYCNEHVEIARALHTPQRVLPLKKPPMKSLRKKGSGKILTPAK
KSFLRRLFD (SEQ ID NO: 164)
>PGK promoter associated with FANCA gene
GGGGTTGGGGTTGCGCCTTTTCCAAGGCAGCCCTGGGTTTGCGCAGGGACGCGGCTGCTCTGGGCGTGGTTCCG
GGAAACGCAGCGGCGCCGACCCTGGGTCTCGCACATTCTTCACGTCCGTTCGCAGCGTCACCCGGATCTTCGCC
GCTACCCTTGTGGGCCCCCCGGCGACGCTTCCTGCTCCGCCCCTAAGTCGGGAAGGTTCCTTGCGGTTCGCGGC
GTGCCGGACGTGACAAACGGAAGCCGCACGTCTCACTAGTACCCTCGCAGACGGACAGCGCCAGGGAGCAATGG
CAGCGCGCCGACCGCGATGGGCTGTGGCCAATAGCGGCTGCTCAGCGGGGCGCGCCGAGAGCAGCGGCCGGGAA
GGGGCGGTGCGGGAGGCGGGGTGTGGGGCGGTAGTGTGGGCCCTGTTCCTGCCCGCGCGGTGTTCCGCATTCTG
CAAGCCTCCGGAGCGCACGTCGGCAGTCGGCTCCCTCGTTGACCGAATCACCGACCTCTCTCCCCAGGGGGATC
CACCGGTCCGCCAAGGCCATGTCCGACTCGTGGGTCCCGAACTCCGCCTCGGGCCAGGACCCAGGGGGCCGCCG
GAGGGCCTGGGCCGAGCTGCTGGCGGGAAGGGTCAAGAGGGAAAAATATAATCCTGAAAGGGCACAGAAATTAA
AGGAATCAGCTGTGCGCCTCCTGCGAAGCCATCAGGACCTGAATGCCCTTTTGCTTGAGGTAGAAGGTCCACTG
TGTAAAAAATTGTCTCTCAGCAAAGTGATTGACTGTGACAGTTCTGAGGCCTATGCTAATCATTCTAGTTCATT
TATAGGCTCTGCTTTGCAGGATCAAGCCTCAAGGCTGGGGGTTCCCGTGGGTATTCTCTCAGCCGGGATGGTTG
CCTCTAGCGTGGGACAGATCTGCACGGCTCCAGCGGAGACCAGTCACCCTGTGCTGCTGACTGTGGAGCAGAGA
AAGAAGCTGTCTTCCCTGTTAGAGTTTGCTCAGTATTTATTGGCACACAGTATGTTCTCCCGTCTTTCCTTCTG
TCAAGAATTATGGAAAATACAGAGTTCTTTGTTGCTTGAAGCGGTGTGGCATCTTCACGTACAAGGCATTGTGA
GCCTGCAAGAGCTGCTGGAAAGCCATCCCGACATGCATGCTGTGGGATCGTGGCTCTTCAGGAATCTGTGCTGC
CTTTGTGAACAGATGGAAGCATCCTGCCAGCATGCTGACGTCGCCAGGGCCATGCTTTCTGATTTTGTTCAAAT
GTTTGTTTTGAGGGGATTTCAGAAAAACTCAGATCTGAGAAGAACTGTGGAGCCTGAAAAAATGCCGCAGGTCA
CGGTTGATGTACTGCAGAGAATGCTGATTTTTGCACTTGACGCTTTGGCTGCTGGAGTACAGGAGGAGTCCTCC
ACTCACAAGATCGTGAGGTGCTGGTTCGGAGTGTTCAGTGGACACACGCTTGGCAGTGTAATTTCCACAGATCC
TCTGAAGAGGTTCTTCAGTCATACCCTGACTCAGATACTCACTCACAGCCCTGTGCTGAAAGCATCTGATGCTG
TTCAGATGCAGAGAGAGTGGAGCTTTGCGCGGACACACCCTCTGCTCACCTCACTGTACCGCAGGCTCTTTGTG
ATGCTGAGTGCAGAGGAGTTGGTTGGCCATTTGCAAGAAGTTCTGGAAACGCAGGAGGTTCACTGGCAGAGAGT
GCTCTCCTTTGTGTCTGCCCTGGTTGTCTGCTTTCCAGAAGCGCAGCAGCTGCTTGAAGACTGGGTGGCGCGTT
TGATGGCCCAGGCATTCGAGAGCTGCCAGCTGGACAGCATGGTCACTGCGTTCCTGGTTGTGCGCCAGGCAGCA
CTGGAGGGCCCCTCTGCGTTCCTGTCATATGCAGACTGGTTCAAGGCCTCCTTTGGGAGCACACGAGGCTACCA
TGGCTGCAGCAAGAAGGCCCTGGTCTTCCTGTTTACGTTCTTGTCAGAACTCGTGCCTTTTGAGTCTCCCCGGT
ACCTGCAGGTGCACATTCTCCACCCACCCCTGGTTCCCAGCAAGTACCGCTCCCTCCTCACAGACTACATCTCA
TTGGCCAAGACACGGCTGGCCGACCTCAAGGTTTCTATAGAAAACATGGGACTCTACGAGGATTTGTCATCAGC
TGGGGACATTACTGAGCCCCACAGCCAAGCTCTTCAGGATGTTGAAAAGGCCATCATGGTGTTTGAGCATACGG
GGAACATCCCAGTCACCGTCATGGAGGCCAGCATATTCAGGAGGCCTTACTACGTGTCCCACTTCCTCCCCGCC
CTGCTCACACCTCGAGTGCTCCCCAAAGTCCCTGACTCCCGTGTGGCGTTTATAGAGTCTCTGAAGAGAGCAGA
TAAAATCCCCCCATCTCTGTACTCCACCTACTGCCAGGCCTGCTCTGCTGCTGAAGAGAAGCCAGAAGATGCAG
CCCTGGGAGTGAGGGCAGAACCCAACTCTGCTGAGGAGCCCCTGGGACAGCTCACAGCTGCACTGGGAGAGCTG
AGAGCCTCCATGACAGACCCCAGCCAGCGTGATGTTATATCGGCACAGGTGGCAGTGATTTCTGAAAGACTGAG
GGCTGTCCTGGGCCACAATGAGGATGACAGCAGCGTTGAGATATCAAAGATTCAGCTCAGCATCAACACGCCGA
GACTGGAGCCACGGGAACACATTGCTGTGGACCTCCTGCTGACGTCTTTCTGTCAGAACCTGATGGCTGCCTCC
AGTGTCGCTCCCCCGGAGAGGCAGGGTCCCTGGGCTGCCCTCTTCGTGAGGACCATGTGTGGACGTGTGCTCCC
TGCAGTGCTCACCCGGCTCTGCCAGCTGCTCCGTCACCAGGGCCCGAGCCTGAGTGCCCCACATGTGCTGGGGT
TGGCTGCCCTGGCCGTGCACCTGGGTGAGTCCAGGTCTGCGCTCCCAGAGGTGGATGTGGGTCCTCCTGCACCT
GGTGCTGGCCTTCCTGTCCCTGCGCTCTTTGACAGCCTCCTGACCTGTAGGACGAGGGATTCCTTGTTCTTCTG
CCTGAAATTTTGTACAGCAGCAATTTCTTACTCTCTCTGCAAGTTTTCTTCCCAGTCACGAGATACTTTGTGCA
GCTGCTTATCTCCAGGCCTTATTAAAAAGTTTCAGTTCCTCATGTTCAGATTGTTCTCAGAGGCCCGACAGCCT
CTTTCTGAGGAGGACGTAGCCAGCCTTTCCTGGAGACCCTTGCACCTTCCTTCTGCAGACTGGCAGAGAGCTGC
CCTCTCTCTCTGGACACACAGAACCTTCCGAGAGGTGTTGAAAGAGGAAGATGTTCACTTAACTTACCAAGACT
GGTTACACCTGGAGCTGGAAATTCAACCTGAAGCTGATGCTCTTTCAGATACTGAACGGCAGGACTTCCACCAG
TGGGCGATCCATGAGCACTTTCTCCCTGAGTCCTCGGCTTCAGGGGGCTGTGACGGAGACCTGCAGGCTGCGTG
TACCATTCTTGTCAACGCACTGATGGATTTCCACCAAAGCTCAAGGAGTTATGACCACTCAGAAAATTCTGATT
TGGTCTTTGGTGGCCGCACAGGAAATGAGGATATTATTTCCAGATTGCAGGAGATGGTAGCTGACCTGGAGCTG
CAGCAAGACCTCATAGTGCCTCTCGGCCACACCCCTTCCCAGGAGCACTTCCTCTTTGAGATTTTCCGCAGACG
GCTCCAGGCTCTGACAAGCGGGTGGAGCGTGGCTGCCAGCCTTCAGAGACAGAGGGAGCTGCTAATGTACAAAC
GGATCCTCCTCCGCCTGCCTTCGTCTGTCCTCTGCGGCAGCAGCTTCCAGGCAGAACAGCCCATCACTGCCAGA
TGCGAGCAGTTCTTCCACTTGGTCAACTCTGAGATGAGAAACTTCTGCTCCCACGGAGGTGCCCTGACACAGGA
CATCACTGCCCACTTCTTCAGGGGCCTCCTGAACGCCTGTCTGCGGAGCAGAGACCCCTCCCTGATGGTCGACT
TCATACTGGCCAAGTGCCAGACGAAATGCCCCTTAATTTTGACCTCTGCTCTGGTGTGGTGGCCGAGCCTGGAG
CCTGTGCTGCTCTGCCGGTGGAGGAGACACTGCCAGAGCCCGCTGCCCCGGGAACTGCAGAAGCTACAAGAAGG
CCGGCAGTTTGCCAGCGATTTCCTCTCCCCTGAGGCTGCCTCCCCAGCACCCAACCCGGACTGGCTCTCAGCTG
CTGCACTGCACTTTGCGATTCAACAAGTCAGGGAAGAAAACATCAGGAAGCAGCTAAAGAAGCTGGACTGCGAG
AGAGAGGAGCTATTGGTTTTCCTTTTCTTCTTCTCCTTGATGGGCCTGCTGTCGTCACATCTGACCTCAAATAG
CACCACAGACCTGCCAAAGGCTTTCCACGTTTGTGCAGCAATCCTCGAGTGTTTAGAGAAGAGGAAGATATCCT
GGCTGGCACTCTTTCAGTTGACAGAGAGTGACCTCAGGCTGGGGCGGCTCCTCCTCCGTGTGGCCCCGGATCAG
CACACCAGGCTGCTGCCTTTCGCTTTTTACAGTCTTCTCTCCTACTTCCATGAAGACGCGGCCATCAGGGAAGA
GGCCTTCCTGCATGTTGCTGTGGACATGTACTTGAAGCTGGTCCAGCTCTTCGTGGCTGGGGATACAAGCACAG
TTTCACCTCCAGCTGGCAGGAGCCTGGAGCTCAAGGGTCAGGGCAACCCCGTGGAACTGATAACAAAAGCTCGT
CTTTTTCTGCTGCAGTTAATACCTCGGTGCCCGAAAAAGAGCTTCTCACACGTGGCAGAGCTGCTGGCTGATCG
TGGGGACTGCGACCCAGAGGTGAGCGCCGCCCTCCAGAGCAGACAGCAGGCTGCCCCTGACGCTGACCTGTCCC
AGGAGCCTCATCTCTTCTGA (SEQ ID NO: 165)
>506 PGK.FancA
TCGCGCGTTCTCGAGGAGCTTGGCCCATTGCATACGTTGTATCCATATCATAATATGTACATTTATATTGGCTC
ATGTCCAACATTACCGCCATGTTGACATTGATTATTGACTAGTTATTAATAGTAATCAATTACGGGGTCATTAG
TTCATAGCCCATATATGGAGTTCCGCGTTACATAACTTACGGTAAATGGCCCGCCTGGCTGACCGCCCAACGAC
CCCCGCCCATTGACGTCAATAATGACGTATGTTCCCATAGTAACGCCAATAGGGACTTTCCATTGACGTCAATG
GGTGGAGTATTTACGCTAAACTGCCCACTTGGCAGTACATCAAGTGTATCATATGCCAAGTACGCCCCCTATTG
ACGTCAATGACGGTAAATGGCCCGCCTGGCATTATGCCCAGTACATGACCTTATGGGACTTTCCTACTTGGCAG
TACATCTACGTATTAGTCATCGCTATTACCATGGTGATGCGGTTTTGGCAGTACATCAATGGGCGTGGATAGCG
GTTTGACTCACGGGGATTTCCAAGTCTCCACCCCATTGACGCAAATGGGCGGTAGGCGTGTACGGTGGGAGGTC
TATATAAGCAGAGCTTCTAGATTGTACGGGAGCTCTCTCTTCACTACTCGCTGCGTCGAGAGTGTACGAGACTC
TCCAGGTTTGGTAAGAAATATTTTATATTGTTATAATGTTACTATGATCCATTAACACTCTGCTTATAGATTGT
AAGGGTGATTGCAATGCTTTCTGCATAAAACTTTGGTTTTCTTGTTAATCAATAAACCGACTTGATTCGAGAAC
CTACTCATATATTATTGTCTCTTTTATACTTTATTAAGTAAAAGGATTTGTATATTAGCCTTGCTAAGGGAGAC
ATCTAGTGATATAAGTGTGAACTACACTTATCTTAAATGATGTAACTCCTTAGGATAATCAATATACAAAATTC
CATGACAATTGGCGCCCAACGTGGGGCTCGAATATAAGTCGGGTTTATTTGTAAATTATCCCTAGGGACCTCCG
AGCATAGCGGGAGGCATATAAAAGCCAATAGACAATGGCTAGCAGGAAGTAATGTTGAAGAATATGAACTTGAT
GTTGAAGCTCTGGTTGTAATTTTAAGAGATAGAAATATACCAAGAAATCCTTTACATGGAGAAGTTATAGGTCT
TCGCCTTACTGAAGGATGGTGGGGACAAATTGAGAGATTTCAGATGGTACGTTGATTCGAATTAAGGCTATGGA
TTTGGCCATGGGACAAGAAATATTAGTTTATAGTCCCATTGTATCTATGACTAAAATACAAAAAACTCCACTAC
CAGAAAGAAAAGCTTTACCCATTAGATGGATAACATGGATGACTTATTTAGAAGATCCAAGAATCCAATTTCAT
TATGATAAAACCTTACCAGAACTTAAGCATATTCCAGATGTATATACATCTAGTCAGTCTCCTGTTAAACATCC
TTCTCAATATGAAGGAGTGTTTTATACTGATGGCTCGGCCATCAAAAGTCCTGATCCTACAAAAAGCAATAATG
CTGGCATGGGAATAGTACATGCCACATACAAACCTGAATATCAAGTTTTGAATCAATGGTCAATACCACTAGGT
AATCATACTGCTCAGATGGCTGAAATAGCTGCAGTTGAATTTGCCTGTAAAAAAGCTTTAAAAATACCTGGTCC
TGTATTAGTTATAACTGATAGTTTCTATGTAGCAGAAAGTGCTAATAAAGAATTACCATACTGGAAATCTAATG
GGTTTGTTAATAATAAGAAAAAGCCTCTTAAACATATCTCCAAATGGAAATCTATTGCTGAGTGTTTATCTATG
AAACCAGACATTACTATTCAACATGAAAAAGGCATCAGCCTACAAATACCAGTATTCATACTGAAAGGCAATGC
CCTAGCAGATAAGCTTGCCACCCAAGGAAGTTATGTGGTTAATTGTAATACCAAAAAACCAAACCTGGATGCAG
AGTTGGATCAATTATTACAGGGTCATTATATAAAAGGATATCCCAAACAATATACATATTTTTTAGAAGATGGC
AAAGTAAAAGTTTCCAGACCTGAAGGGGTTAAAATTATTCCCCCTCAGTCAGACAGACAAAAAATTGTGCTTCA
AGCCCACAATTTGGCTCACACCGGACGTGAAGCCACTCTTTTAAAAATTGCCAACCTTTATTGGTGGCCAAATA
TGAGAAAGGATGTGGTTAAACAACTAGGACGCTGTCAACAGTGTTTAATCACAAATGCTTCCAACAAAGCCTCT
GGTCCTATTCTAAGACCAGATAGGCCTCAAAAACCTTTTGATAAATTCTTTATTGACTATATTGGACCTTTGCC
ACCTTCACAGGGATACCTATATGTATTAGTAGTTGTTGATGGAATGACAGGATTCACTTGGTTATACCCCACTA
AGGCTCCTTCTACTAGCGCAACTGTTAAATCTCTCAATGTACTCACTAGTATTGCAATTCCAAAGGTGATTCAC
TCTGATCAAGGTGCAGCATTCACTTCTTCAACCTTTGCTGAATGGGCAAAGGAAAGAGGTATACATTTGGAATT
CAGTACTCCTTATCACCCCCAAAGTGGTAGTAAGGTGGAAAGGAAAAATAGTGATATAAAACGACTTTTAACTA
AACTGCTAGTAGGAAGACCCACAAAGTGGTATGACCTATTGCCTGTTGTACAACTTGCTTTAAACAACACCTAT
AGCCCTGTATTAAAATATACTCCACATCAACTCTTATTTGGTATAGATTCAAATACTCCATTTGCAAATCAAGA
TACACTTGACTTGACCAGAGAAGAAGAACTTTCTCTTTTACAGGAAATTCGTACTTCTTTATACCATCCATCCA
CCCCTCCAGCCTCCTCTCGTTCCTGGTCTCCTGTTGTTGGCCAATTGGTCCAGGAGAGGGTGGCTAGGCCTGCT
TCTTTGAGACCTCGTTGGCATAAACCGTCTACTGTACTTAAGGTGTTGAATCCAAGGACTGTTGTTATTTTGGA
CCATCTTGGCAACAACAGAACTGTAAGTATAGATAATTTAAAACCTACTTCTCATCAGAATGGCACCACCAATG
ACACTGCAACAATGGATCATTTGGAAAAAAATGAATAAAGCGCATGAGGCACTTCAAAATACAACAACTGTGAC
TGAACAGCAGAAGGAACAAATTATACTGGACATTCAAAATGAAGAAGTACAACCAACTAGGAGAGATAAATTTA
GATATCTGCTTTATACTTGTTGTGCTACTAGCTCAAGAGTATTGGCCTGGATGTTTTTAGTTTGTATATTGTTA
ATCATTGTTTTGGTTTCATGCTTTGTGACTATATCCAGAATACAATGGAATAAGGATATTCAGGTATTAGGACC
TGTAATAGACTGGAATGTTACTCAAAGAGCTGTTTATCAACCCTTACAGACTAGAAGGATTGCACGTTCCCTTA
GAATGCAGCATCCTGTTCCAAAATATGTGGAGGTAAATATGACTAGTATTCCACAAGGTGTATACTATGAACCC
CATCCGGCGCGCCAGATCTGCATGCCACGGGGTTGGGGTTGCGCCTTTTCCAAGGCAGCCCTGGGTTTGCGCAG
GGACGCGGCTGCTCTGGGCGTGGTTCCGGGAAACGCAGCGGCGCCGACCCTGGGTCTCGCACATTCTTCACGTC
CGTTCGCAGCGTCACCCGGATCTTCGCCGCTACCCTTGTGGGCCCCCCGGCGACGCTTCCTGCTCCGCCCCTAA
GTCGGGAAGGTTCCTTGCGGTTCGCGGCGTGCCGGACGTGACAAACGGAAGCCGCACGTCTCACTAGTACCCTC
GCAGACGGACAGCGCCAGGGAGCAATGGCAGCGCGCCGACCGCGATGGGCTGTGGCCAATAGCGGCTGCTCAGC
GGGGCGCGCCGAGAGCAGCGGCCGGGAAGGGGCGGTGCGGGAGGCGGGGTGTGGGGCGGTAGTGTGGGCCCTGT
TCCTGCCCGCGCGGTGTTCCGCATTCTGCAAGCCTCCGGAGCGCACGTCGGCAGTCGGCTCCCTCGTTGACCGA
ATCACCGACCTCTCTCCCCAGGGGGATCCACCGGTCCGCCAAGGCCATGTCCGACTCGTGGGTCCCGAACTCCG
CCTCGGGCCAGGACCCAGGGGGCCGCCGGAGGGCCTGGGCCGAGCTGCTGGCGGGAAGGGTCAAGAGGGAAAAA
TATAATCCTGAAAGGGCACAGAAATTAAAGGAATCAGCTGTGCGCCTCCTGCGAAGCCATCAGGACCTGAATGC
CCTTTTGCTTGAGGTAGAAGGTCCACTGTGTAAAAAATTGTCTCTCAGCAAAGTGATTGACTGTGACAGTTCTG
AGGCCTATGCTAATCATTCTAGTTCATTTATAGGCTCTGCTTTGCAGGATCAAGCCTCAAGGCTGGGGGTTCCC
GTGGGTATTCTCTCAGCCGGGATGGTTGCCTCTAGCGTGGGACAGATCTGCACGGCTCCAGCGGAGACCAGTCA
CCCTGTGCTGCTGACTGTGGAGCAGAGAAAGAAGCTGTCTTCCCTGTTAGAGTTTGCTCAGTATTTATTGGCAC
ACAGTATGTTCTCCCGTCTTTCCTTCTGTCAAGAATTATGGAAAATACAGAGTTCTTTGTTGCTTGAAGCGGTG
TGGCATCTTCACGTACAAGGCATTGTGAGCCTGCAAGAGCTGCTGGAAAGCCATCCCGACATGCATGCTGTGGG
ATCGTGGCTCTTCAGGAATCTGTGCTGCCTTTGTGAACAGATGGAAGCATCCTGCCAGCATGCTGACGTCGCCA
GGGCCATGCTTTCTGATTTTGTTCAAATGTTTGTTTTGAGGGGATTTCAGAAAAACTCAGATCTGAGAAGAACT
GTGGAGCCTGAAAAAATGCCGCAGGTCACGGTTGATGTACTGCAGAGAATGCTGATTTTTGCACTTGACGCTTT
GGCTGCTGGAGTACAGGAGGAGTCCTCCACTCACAAGATCGTGAGGTGCTGGTTCGGAGTGTTCAGTGGACACA
CGCTTGGCAGTGTAATTTCCACAGATCCTCTGAAGAGGTTCTTCAGTCATACCCTGACTCAGATACTCACTCAC
AGCCCTGTGCTGAAAGCATCTGATGCTGTTCAGATGCAGAGAGAGTGGAGCTTTGCGCGGACACACCCTCTGCT
CACCTCACTGTACCGCAGGCTCTTTGTGATGCTGAGTGCAGAGGAGTTGGTTGGCCATTTGCAAGAAGTTCTGG
AAACGCAGGAGGTTCACTGGCAGAGAGTGCTCTCCTTTGTGTCTGCCCTGGTTGTCTGCTTTCCAGAAGCGCAG
CAGCTGCTTGAAGACTGGGTGGCGCGTTTGATGGCCCAGGCATTCGAGAGCTGCCAGCTGGACAGCATGGTCAC
TGCGTTCCTGGTTGTGCGCCAGGCAGCACTGGAGGGCCCCTCTGCGTTCCTGTCATATGCAGACTGGTTCAAGG
CCTCCTTTGGGAGCACACGAGGCTACCATGGCTGCAGCAAGAAGGCCCTGGTCTTCCTGTTTACGTTCTTGTCA
GAACTCGTGCCTTTTGAGTCTCCCCGGTACCTGCAGGTGCACATTCTCCACCCACCCCTGGTTCCCAGCAAGTA
CCGCTCCCTCCTCACAGACTACATCTCATTGGCCAAGACACGGCTGGCCGACCTCAAGGTTTCTATAGAAAACA
TGGGACTCTACGAGGATTTGTCATCAGCTGGGGACATTACTGAGCCCCACAGCCAAGCTCTTCAGGATGTTGAA
AAGGCCATCATGGTGTTTGAGCATACGGGGAACATCCCAGTCACCGTCATGGAGGCCAGCATATTCAGGAGGCC
TTACTACGTGTCCCACTTCCTCCCCGCCCTGCTCACACCTCGAGTGCTCCCCAAAGTCCCTGACTCCCGTGTGG
CGTTTATAGAGTCTCTGAAGAGAGCAGATAAAATCCCCCCATCTCTGTACTCCACCTACTGCCAGGCCTGCTCT
GCTGCTGAAGAGAAGCCAGAAGATGCAGCCCTGGGAGTGAGGGCAGAACCCAACTCTGCTGAGGAGCCCCTGGG
ACAGCTCACAGCTGCACTGGGAGAGCTGAGAGCCTCCATGACAGACCCCAGCCAGCGTGATGTTATATCGGCAC
AGGTGGCAGTGATTTCTGAAAGACTGAGGGCTGTCCTGGGCCACAATGAGGATGACAGCAGCGTTGAGATATCA
AAGATTCAGCTCAGCATCAACACGCCGAGACTGGAGCCACGGGAACACATTGCTGTGGACCTCCTGCTGACGTC
TTTCTGTCAGAACCTGATGGCTGCCTCCAGTGTCGCTCCCCCGGAGAGGCAGGGTCCCTGGGCTGCCCTCTTCG
TGAGGACCATGTGTGGACGTGTGCTCCCTGCAGTGCTCACCCGGCTCTGCCAGCTGCTCCGTCACCAGGGCCCG
AGCCTGAGTGCCCCACATGTGCTGGGGTTGGCTGCCCTGGCCGTGCACCTGGGTGAGTCCAGGTCTGCGCTCCC
AGAGGTGGATGTGGGTCCTCCTGCACCTGGTGCTGGCCTTCCTGTCCCTGCGCTCTTTGACAGCCTCCTGACCT
GTAGGACGAGGGATTCCTTGTTCTTCTGCCTGAAATTTTGTACAGCAGCAATTTCTTACTCTCTCTGCAAGTTT
TCTTCCCAGTCACGAGATACTTTGTGCAGCTGCTTATCTCCAGGCCTTATTAAAAAGTTTCAGTTCCTCATGTT
CAGATTGTTCTCAGAGGCCCGACAGCCTCTTTCTGAGGAGGACGTAGCCAGCCTTTCCTGGAGACCCTTGCACC
TTCCTTCTGCAGACTGGCAGAGAGCTGCCCTCTCTCTCTGGACACACAGAACCTTCCGAGAGGTGTTGAAAGAG
GAAGATGTTCACTTAACTTACCAAGACTGGTTACACCTGGAGCTGGAAATTCAACCTGAAGCTGATGCTCTTTC
AGATACTGAACGGCAGGACTTCCACCAGTGGGCGATCCATGAGCACTTTCTCCCTGAGTCCTCGGCTTCAGGGG
GCTGTGACGGAGACCTGCAGGCTGCGTGTACCATTCTTGTCAACGCACTGATGGATTTCCACCAAAGCTCAAGG
AGTTATGACCACTCAGAAAATTCTGATTTGGTCTTTGGTGGCCGCACAGGAAATGAGGATATTATTTCCAGATT
GCAGGAGATGGTAGCTGACCTGGAGCTGCAGCAAGACCTCATAGTGCCTCTCGGCCACACCCCTTCCCAGGAGC
ACTTCCTCTTTGAGATTTTCCGCAGACGGCTCCAGGCTCTGACAAGCGGGTGGAGCGTGGCTGCCAGCCTTCAG
AGACAGAGGGAGCTGCTAATGTACAAACGGATCCTCCTCCGCCTGCCTTCGTCTGTCCTCTGCGGCAGCAGCTT
CCAGGCAGAACAGCCCATCACTGCCAGATGCGAGCAGTTCTTCCACTTGGTCAACTCTGAGATGAGAAACTTCT
GCTCCCACGGAGGTGCCCTGACACAGGACATCACTGCCCACTTCTTCAGGGGCCTCCTGAACGCCTGTCTGCGG
AGCAGAGACCCCTCCCTGATGGTCGACTTCATACTGGCCAAGTGCCAGACGAAATGCCCCTTAATTTTGACCTC
TGCTCTGGTGTGGTGGCCGAGCCTGGAGCCTGTGCTGCTCTGCCGGTGGAGGAGACACTGCCAGAGCCCGCTGC
CCCGGGAACTGCAGAAGCTACAAGAAGGCCGGCAGTTTGCCAGCGATTTCCTCTCCCCTGAGGCTGCCTCCCCA
GCACCCAACCCGGACTGGCTCTCAGCTGCTGCACTGCACTTTGCGATTCAACAAGTCAGGGAAGAAAACATCAG
GAAGCAGCTAAAGAAGCTGGACTGCGAGAGAGAGGAGCTATTGGTTTTCCTTTTCTTCTTCTCCTTGATGGGCC
TGCTGTCGTCACATCTGACCTCAAATAGCACCACAGACCTGCCAAAGGCTTTCCACGTTTGTGCAGCAATCCTC
GAGTGTTTAGAGAAGAGGAAGATATCCTGGCTGGCACTCTTTCAGTTGACAGAGAGTGACCTCAGGCTGGGGCG
GCTCCTCCTCCGTGTGGCCCCGGATCAGCACACCAGGCTGCTGCCTTTCGCTTTTTACAGTCTTCTCTCCTACT
TCCATGAAGACGCGGCCATCAGGGAAGAGGCCTTCCTGCATGTTGCTGTGGACATGTACTTGAAGCTGGTCCAG
CTCTTCGTGGCTGGGGATACAAGCACAGTTTCACCTCCAGCTGGCAGGAGCCTGGAGCTCAAGGGTCAGGGCAA
CCCCGTGGAACTGATAACAAAAGCTCGTCTTTTTCTGCTGCAGTTAATACCTCGGTGCCCGAAAAAGAGCTTCT
CACACGTGGCAGAGCTGCTGGCTGATCGTGGGGACTGCGACCCAGAGGTGAGCGCCGCCCTCCAGAGCAGACAG
CAGGCTGCCCCTGACGCTGACCTGTCCCAGGAGCCTCATCTCTTCTGACGGGACCTGCCACTGCACACCAGCCC
AGCTCCCGTGTAAATAATTTATTACAAGCATAACATGGAGCTCTTGTTGCACTAAAAAGTGGATTACAAATCTC
CTCGACTGCTTTAGTGGGGAAAGGAATCAATTATTTATGAACTGTCCGGCCCCGAGTCACTCAGCGTTTGCGGG
AAAATAAACCACTGGTCCCAGAGCAGAGGAAGGCTACTTGAGCCGGACACCAAGCCCGCCTCCAGCACCAAGGG
CGGGCAGCACCCTCCGACCCTCCCATGCGGGTGCACACGAAGGGTGAGGCTGACACAGCCACTGCGGAGTCCAG
GCTGCTAGAGGTGCTCATCCTCACTGCCGTCCTCAGGTGGGTTCGGGCTTCACCGCCTGGCCCTCTGTGGTCAC
AGAGGGGCTCGGTGGCCCAGGTGGTGGTTCCGCCTCCAGGGGCAGGGCCTTGTCCTGGGTCTGTGTCAGCGGGT
GCACCATGGACATGTGTACAAGTAAAGCGGCCGCGTCGAGGGCTGCAGGAATTCGAGCATCTTACCGCCATTTA
TTCCCATATTTGTTCTGTTTTTCTTGATTTGGGTATACATTTAAATGTTAATAAAACAAAATGGTGGGGCAATC
ATTTACATTTTTAGGGATATGTAATTACTAGTTCAGGTGTATTGCCACAAGACAAACATGTTAAGAAACTTTCC
CGTTATTTACGCTCTGTTCCTGTTAATCAACCTCTGGATTACAAAATTTGTGAAAGATTGACTGATATTCTTAA
CTATGTTGCTCCTTTTACGCTGTGTGGATATGCTGCTTTATAGCCTCTGTATCTAGCTATTGCTTCCCGTACGG
CTTTCGTTTTCTCCTCCTTGTATAAATCCTGGTTGCTGTCTCTTTTAGAGGAGTTGTGGCCCGTTGTCCGTCAA
CGTGGCGTGGTGTGCTCTGTGTTTGCTGACGCAACCCCCACTGGCTGGGGCATTGCCACCACCTGTCAACTCCT
TTCTGGGACTTTCGCTTTCCCCCTCCCGATCGCCACGGCAGAACTCATCGCCGCCTGCCTTGCCCGCTGCTGGA
CAGGGGCTAGGTTGCTGGGCACTGATAATTCCGTGGTGTTGTCGGGGAAGCTGACGTCCTTTCGAATTCGATAT
CAAGCTTATCGATACCGTCGACGGTTACCAAGCAGCTATGGAAGCTTATGGACCTCAGAGAGGAAGTAACGAGG
AGAGGGTGTGGTGGAATGCCACTAGAAACCAGGGAAAACAAGGAGGAGAGTATTACAGGGAAGGAGGTGAAGAA
CCTCATTACCCAAATACTCCTGCTCCTCATAGACGTACCTGGGATGAGAGACACAAGGTTCTTAAATTGTCCTC
ATTCGCTACTCCCTCTGACATCCAACGCTGGGCTACTAACTCTAGATTGTACGGGAGCTCTCTTCACTACTCGC
TGCGTCGAGAGTGTACGAGACTCTCCAGGTTTGGTAAGAAATATTTTATATTGTTATAATGTTACTATGATCCA
TTAACACTCTGCTTATAGATTGTAAGGGTGATTGCAATGCTTTCTGCATAAAACTTTGGTTTTCTTGTTAATCA
ATAAACCGACTTGATTCGAGAACCTACTCATATATTATTGTCTCTTTTATACTTTATTAAGTAAAAGGATTTGT
ATATTAGCCTTGCTAAGGGAGACATCTAGTGATATAAGTGTGAACTACACTTATCTTAAATGATGTAACTCCTT
AGGATAATCAATATACAAAATTCCATGACAATTGGCGATACCCAGCTGCGCTCTTCCGCTTCCTCGCTCACTGA
CTCGCTGCGCTCGGTCGTTCGGCTGCGGCGAGCGGTATCAGCTCACTCAAAGGCGGTAATACGGTTATCCACAG
AATCAGGGGATAACGCAGGAAAGAACATGTGAGCAAAAGGCCAGCAAAAGGCCAGGAACCGTAAAAAGGCCGCG
TTGCTGGCGTTTTTCCATAGGCTCCGCCCCCCTGACGAGCATCACAAAAATCGACGCTCAAGTCAGAGGTGGCG
AAACCCGACAGGACTATAAAGATACCAGGCGTTTCCCCCTGGAAGCTCCCTCGTGCGCTCTCCTGTTCCGACCC
TGCCGCTTACCGGATACCTGTCCGCCTTTCTCCCTTCGGGAAGCGTGGCGCTTTCTCATAGCTCACGCTGTAGG
TATCTCAGTTCGGTGTAGGTCGTTCGCTCCAAGCTGGGCTGTGTGCACGAACCCCCCGTTCAGCCCGACCGCTG
CGCCTTATCCGGTAACTATCGTCTTGAGTCCAACCCGGTAAGACACGACTTATCGCCACTGGCAGCAGCCACTG
GTAACAGGATTAGCAGAGCGAGGTATGTAGGCGGTGCTACAGAGTTCTTGAAGTGGTGGCCTAACTACGGCTAC
ACTAGAAGAACAGTATTTGGTATCTGCGCTCTGCTGAAGCCAGTTACCTTCGGAAAAAGAGTTGGTAGCTCTTG
ATCCGGCAAACAAACCACCGCTGGTAGCGGTGGTTTTTTTGTTTGCAAGCAGCAGATTACGCGCAGAAAAAAAG
GATCTCAAGAAGATCCTTTGATCTTTTCTACGGGGTCTGACGCTCAGTGGAACGAAAACTCACGTTAAGGGATT
TTGGTCATGAGATTATCAAAAAGGATCTTCACCTAGATCCTTTTAAATTAAAAATGAAGTTTTAAATCAATCTA
AAGTATATATGAGTAAACTTGGTCTGACAGTTACCAATGCTTAATCAGTGAGGCACCTATCTCAGCGATCTGTC
TATTTCGTTCATCCATAGTTGCCTGACTCCCCGTCGTGTAGATAACTACGATACGGGAGGGCTTACCATCTGGC
CCCAGTGCTGCAATGATACCGCGAGACCCACGCTCACCGGCTCCAGATTTATCAGCAATAAACCAGCCAGCCGG
AAGGGCCGAGCGCAGAAGTGGTCCTGCAACTTTATCCGCCTCCATCCAGTCTATTAATTGTTGCCGGGAAGCTA
GAGTAAGTAGTTCGCCAGTTAATAGTTTGCGCAACGTTGTTGCCATTGCTACAGGCATCGTGGTGTCACGCTCG
TCGTTTGGTATGGCTTCATTCAGCTCCGGTTCCCAACGATCAAGGCGAGTTACATGATCCCCCATGTTGTGCAA
AAAAGCGGTTAGCTCCTTCGGTCCTCCGATCGTTGTCAGAAGTAAGTTGGCCGCAGTGTTATCACTCATGGTTA
TGGCAGCACTGCATAATTCTCTTACTGTCATGCCATCCGTAAGATGCTTTTCTGTGACTGGTGAGTACTCAACC
AAGTCATTCTGAGAATAGTGTATGCGGCGACCGAGTTGCTCTTGCCCGGCGTCAATACGGGATAATACCGCGCC
ACATAGCAGAACTTTAAAAGTGCTCATCATTGGAAAACGTTCTTCGGGGCGAAAACTCTCAAGGATCTTACCGC
TGTTGAGATCCAGTTCGATGTAACCCACTCGTGCACCCAACTGATCTTCAGCATCTTTTACTTTCACCAGCGTT
TCTGGGTGAGCAAAAACAGGAAGGCAAAATGCCGCAAAAAAGGGAATAAGGGCGACACGGAAATGTTGAATACT
CATACTCTTCCTTTTTCAATATTATTGAAGCATTTATCAGGGTTATTGTCTCATGAGCGGATACATATTTGAAT
GTATTTAGAAAAATAAACAAATAGGGGTTCCGCGCACATTTCCCCGAAAAGTGCCACCTGACGTCTAAGAAACC
ATTATTATCATGACATTAACCTATAAAAATAGGCGTATCACGAGGCCCTTTCGTC (SEQ ID NO: 166)
LTG 1906 EF1a-VH-4 CD33-CD8 TM-41BB-CD3 zeta
MLLLVTSLLLCELPHPAFLLIPEVQLVESGGGLVQPGGSLRLSCAASGFTFSSYGMSWVRQAPRQGLEWVANIK
QDGSEKYYADSVKGRFTISRDNSKNTLYLQMNSLRAEDTATYYCAKENVDWGQGTLVTVSSAAATTTPAPRPPT
PAPTIASQPLSLRPEACRPAAGGAVHTRGLDFACDIYIWAPLAGTCGVLLLSLVITLYCKRGRKKLLYIFKQPF
MRPVQTTQEEDGCSCRFPEEEEGGCELRVKFSRSADAPAYQQGQNQLYNELNLGRREEYDVLDKRRGRDPEMGG
KPRRKNPQEGLYNELQKDKMAEAYSEIGMKGERRRGKGHDGLYQGLSTATKDTYDALHMQALPPR (SEQ ID
NO: 167)
LTG 1905 EF1a VH-2 CD33-CD8 TM-41BB-CD3 zeta
MLLLVTSLLLCELPHPAFLLIPEVQLVESGGGLVQPGGSLRLSCAASGFTFSSYGMSWVRQAPRKGLEWIGEIN
HSGSTNYNPSLKSRVTISRDNSKNTLYLQMNSLRAEDTATYYCARPLNYYYYYMDVWGKGTTVTVSSAAATTTP
APRPPTPAPTIASQPLSLRPEACRPAAGGAVHTRGLDFACDIYIWAPLAGTCGVLLLSLVITLYCKRGRKKLLY
IFKQPFMRPVQTTQEEDGCSCRFPEEEEGGCELRVKFSRSADAPAYQQGQNQLYNELNLGRREEYDVLDKRRGR
DPEMGGKPRRKNPQEGLYNELQKDKMAEAYSEIGMKGERRRGKGHDGLYQGLSTATKDTYDALHMQALPPR
(SEQ ID NO: 168)
Full length Macaca mulatta
MPLLLLPLLWAGALAMDPRVRLEVQESVTVQEGLCVLVPCTFFHPVPYHTRNSPVHGYWFREGAIVSLDSPVAT
NKLDQEVREETQGRFRLLGDPSRNNCSLSIVDTRRRDNGSYFFRMEKGSTKYSYKSTQLSVHVTDLTHRPQILI
PGALDPDHSKNLTCSVPWACEQGTPPIFSWMSAAPTSLGLRTTHSSVLIITPRPQDHGTNLTCQVKFPGAGVTT
ERTIQLNVSYASQNPRTDIFLGDGSGKQGVVQGAIGGAGVTVLLALCLCLIFFTVKTHRRKAARTAVGRIDTHP
ATGPTSSKHQKKSKLHGATETSGCSGTTLTVEMDEELHYASLNEHGMNPSEDTSTEYSEVRTQ (SEQ ID
NO: 169)
Full length Macaca fascicularis
MPLLLLPLLWAGALAMDPRVRLEVQESVTVQEGLCVLVPCTFFHPVPYHTRNSPVHGYWFREGAIVSLDSPVAT
NKLDQEVQEETQGRFRLLGDPSRNNCSLSIVDARRRDNGSYFFRMEKGSTKYSYKSTQLSVHVTDLTHRPQILI
PGALDPDHSKNLTCSVPWACEQGTPPIFSWMSAAPTSLGLRTTHSSVLIITPRPQDHGTNLTCQVKFPGAGVTT
ERTIQLNVSYASQNPRTDIFLGDGSGKQGVVQGAIGGAGVTVLLALCLCLIFFTVKTHRRKAARTAVGRIDTHP
ATGPTSSKHQKKSKLHGATETSGCSGTTLTVEMDEELHYASLNFHGMNPSEDTSTEYSEVRTQ (SEQ ID NO:
170)
Full length Mus musculus
MLWPLPLFLLCAGSLAQDLEFQLVAPESVTVEEGLCVHVPCSVFYPSIKLTLGPVTGSWLRKGVSLHEDSPVAT
SDPRQLVQKATQGRFQLLGDPQKHDCSLFIRDAQKNDTGMYFFRVVREPFVRYSYKKSQLSLHVTSLSRTPDII
IPGTLEAGYPSNLTCSVPWACEQGTPPTFSWMSTALTSLSSRTTDSSVLTFTPQPQDHGTKLTCLVTFSGAGVT
VERTIQLNVTRKSGQMRELVLVAVGEATVKLLILGLCLVFLIVMFCRRKTTKLSVHMGCENPIKRQEAITSYNH
CLSPTASDAVTPGCSIHRLISRTPRCTAILRIQDPYRRTHLRNRAVSTLRFPWISWEGSLRSTQRSKCTKLCSP
VKNLCPLWLPVDNSCIPLIPEWVMLLCVSLTLS (SEQ ID NO: 171)
The nucleotide sequence of Helper Vector 2: Ad35E4PS2/WL-ps2:

| | |
|---|---|
| Ad35 1-->178 | Start: 2582 End: 2759 |
| loxP | Start: 2768 End: 2801 |
| Ad35 366-->481 | Start: 2808 End: 2923 |
| loxP | Start: 2924 End: 2957 |
| Ad35 3112-->27435 | Start: 2966 End: 27288 |
| lambda-1 | Start: 27343 End: 29812 (Complementary) |
| BGH polyA | Start: 30126 End: 30340 |
| copGFP | Start: 30365 End: 31030 (Complementary) |
| CMV | Start: 31077 End: 31729 (Complementary) |
| lambda-2 | Start: 31781 End: 33310 |
| Ad35 30544-->31879 | Start: 33371 End: 34706 |
| Ad5E4orf6 | Start: 34702 End: 35816 |
| Ad35 32972 -->34794 | Start: 35814 End: 37636 |

taaacttggcgcgccctgagtgatttttctctggtcccgccgcatccataccgccagttgtttaccctca
caacgttccagtaaccgggcatgttcatcatcagtaacccgtatcgtgagcatcctctctcgtttcatcg
gtatcattacccccatgaacagaaatcccccttacacggaggcatcagtgaccaaacaggaaaaaaccgc
ccttaacatggcccgctttatcagaagccagacattaacgcttctggagaaactcaacgagctggacgcg
gatgaacaggcagacatctgtgaatcgcttcacgaccacgctgatgagctttaccgcagctgcctcgcgc
gtttcggtgatgacggtgaaaacctctgacacatgcagctcccggagacggtcacagcttgtctgtaagc
ggatgccgggagcagacaagcccgtcagggcgcgtcagcgggtgttggcgggtgtcggggcgcagccatg
acccagtcacgtagcgatagcggagtgtatactggcttaactatgcggcatcagagcagattgtactgag
agtgcaccatatgcggtgtgaaataccgcacagatgcgtaaggagaaaataccgcatcaggcgctcttcc
gcttcctcgctcactgactcgctgcgctcggtcgttcggctgcggcgagcggtatcagctcactcaaagg
cggtaatacggttatccacagaatcaggggataacgcaggaaagaacatgtgagcaaaaggccagcaaaa
ggccaggaaccgtaaaaaggccgcgttgctggcgtttttccataggctccgcccccctgacgagcatcac
aaaaatcgacgctcaagtcagaggtggcgaaacccgacaggactataaagataccaggcgtttccccctg
gaagctccctcgtgcgctctcctgttccgaccctgccgcttaccggatacctgtccgcctttctcccttc
gggaagcgtggcgctttctcatagctcacgctgtaggtatctcagttcggtgtaggtcgttcgctccaag
ctgggctgtgtgcacgaaccccccgttcagcccgaccgctgcgccttatccggtaactatcgtcttgagt
ccaacccggtaagacacgacttatcgccactggcagcagccactggtaacaggattagcagagcgaggta
tgtaggcggtgctacagagttcttgaagtggtggcctaactacggctacactagaaggacagtatttggt
atctgcgctctgctgaagccagttaccttcggaaaaagagttggtagctcttgatccggcaaacaaacca
ccgctggtagcggtggtttttttgtttgcaagcagcagattacgcgcagaaaaaaaggatctcaagaaga
tcctttgatcttttctacggggtctgacgctcagtggaacgaaaactcacgttaagggattttggtcatg
agattatcaaaaaggatcttcacctagatccttttaaattaaaaatgaagttttaaatcaatctaaagta
tatatgagtaaacttggtctgacagttaccaatgcttaatcagtgaggcacctatctcagcgatctgtct
atttcgttcatccatagttgcctgactccccgtcgtgtagataactacgatacgggagggcttaccatct
ggccccagtgctgcaatgataccgcgagacccacgctcaccggctccagatttatcagcaataaaccagc
cagccggaagggccgagcgcagaagtggtcctgcaactttatccgcctccatccagtctattaattgttg
ccgggaagctagagtaagtagttcgccagttaatagtttgcgcaacgttgttgccattgctgcaggcatc
gtggtgtcacgctcgtcgtttggtatggcttcattcagctccggttcccaacgatcaaggcgagttacat
gatcccccatgttgtgcaaaaaagcggttagctccttcggtcctccgatcgttgtcagaagtaagttggc
cgcagtgttatcactcatggttatggcagcactgcataattctcttactgtcatgccatccgtaagatgc
ttttctgtgactggtgagtactcaaccaagtcattctgagaatagtgtatgcggcgaccgagttgctctt
gcccggcgtcaacacgggataataccgcgccacatagcagaactttaaaagtgctcatcattggaaaacg
ttcttcggggcgaaaactctcaaggatcttaccgctgttgagatccagttcgatgtaacccactcgtgca
cccaactgatcttcagcatcttttactttcaccagcgtttctgggtgagcaaaaacaggaaggcaaaatg
ccgcaaaaaagggaataagggcgacacggaaatgttgaatactcatactcttcctttttcaatattattg
aagcatttatcagggttattgtctcatgagcggatacatatttgaatgtatttagaaaaataaacaaata
ggggttccgcgcacatttccccgaaaagtgccacctgtctagctacgatatcctgtttaaacatcatcaa
taatataccttatagatggaatggtgccaatatgtaaatgaggtgattttaaaaagtgtgggccgtgtgg
tgattggctgtggggttaacggttaaaaggggcggcgcggccgtgggaaaatgacgttttatgggggtgg
agtttttttgcaagttgtcgcgggaaatgatttaaatataacttcgtatagcatacattatacgaagtta
tggatcctagactttgacccattacgtggaggtttcgattaccgtgttttttacctgaatttccgcgtac
cgtgtcaaagtcttctgtttttacgtaggtgtcagctgatcgctagggtatttataacttcgtatagcat
acattatacgaagttatatttaaataggaatgtttatgccttaccagtgtaacatgaatcatgtgaaagt
gttgttggaaccagatgccttttccagaatgagcctaacaggaatctttgacatgaacacgcaaatctgg
aagatcctgaggtatgatgatacgagatcgagggtgcgcgcatgcgaatgcggaggcaagcatgccaggt
tccagccggtgtgtgtagatgtgaccgaagatctcagaccggatcatttggttattgcccgcactggagc
agagttcggatccagtggagaagaaactgactaaggtgagtattgggaaaactttggggtgggattttca
gatggacagattgagtaaaaatttgttttttctgtcttgcagctgacatgagtggaaatgcttcttttaa
ggggggagtcttcagcccttatctgacagggcgtctcccatcctgggcaggagttcgtcagaatgttatg
ggatctactgtggatggaagacccgttcaacccgccaattcttcaacgctgacctatgctactttaagtt
cttcacctttggacgcagctgcagccgctgccgccgcctctgtcgccgctaacactgtgcttggaatggg
ttactatggaagcatcgtggctaattccacttcctctaataacccttctacactgactcaggacaagtta
cttgtccttttggcccagctggaggctttgacccaacgtctgggtgaactttctcagcaggtggccgagt
tgcgagtacaaactgagtctgctgtcggcacggcaaagtctaaataaaaaaaattccagaatcaatgaat
aaataaacgagcttgttgttgatttaaaatcaagtgtttttatttcatttttcgcgcacggtatgccctg
gaccaccgatctcgatcattgagaactcggtggattttttccagaatcctatagaggtgggattgaatgt
ttagatacatgggcattaggccgtctttggggtggagatagctccattgaagggattcatgctccggggt
agtgttgtaaatcacccagtcataacaaggtcgcagtgcatggtgttgcacaatatcttttagaagtagg
ctgattgccacagataagcccttggtgtaggtgtttacaaaccggttgagctgggaggggtgcattcgag
gtgaaattatgtgcattttggattggatttttaagttggcaatattgccgccaagatcccgtcttgggtt
catgttatgaaggactaccaagacggtgtatccggtacatttaggaaatttatcgtgcagcttggatgga
aaagcgtggaaaaatttggagacacccttgtgtcctccgagattttccatgcactcatccatgataatag
caatggggccgtgggcagcggcgcgggcaaacacgttccgtgggtctgacacatcatagttatgttcctg
agttaaatcatcataagccattttaatgaatttggggcggagcgtaccagattggggtatgaatgttcct
tcgggccccggagcatagttcccctcacagatttgcatttcccaagctttcagttctgagggtggaatca
tgtccacctggggggctatgaagaacaccgtttcgggggcgggggtgattagttgggatgatagcaagtt
tctgagcaattgagatttgccacatccggtggggccataaataattccgattacaggttgcaggtggtag
tttagggaacggcaactgccgtcttctcgaagcaagggggccacctcgttcatcatttcccttacatgca
tattttcccgcaccaaatccattaggaggcgctctcctcctagtgatagaagttcttgtagtgaggaaaa
gtttttcagcggttttagaccgtcagecatgggcattttggaaagagtttgetgcaaaagttctagtctg
ttccacagttcagtgatgtgttctatggcatctcgatccagcagacctcctcgtttcgcgggtttggacg
gctcctggagtagggtatgagacgatgggcgtccagcgctgccagggttcggtccttccagggtctcagt
gttcgagtcagggttgtttccgtcacagtgaaggggtgtgcgcctgcttgggcgcttgccagggtgcgct
tcagactcattctgctggtggagaacttctgtcgcttggcgccctgtatgtcggccaagtagcagtttac
catgagttcgtagttgagcgcctcggctgcgtggcctttggcgcggagcttacctttggaagttttcttg
cataccgggcagtataggcatttcagcgcatacagcttgggcgcaaggaaaatggattctggggagtatg
catccgcgccgcaggaggcgcaaacagtttcacattccaccagecaggttaaatccggttcattggggtc
aaaaacaagttttccgccatattttttgatgcgtttcttacctttggtctccataagttcgtgtcctcgt
tgagtgacaaacaggctgtccgtatctccgtagactgattttacaggcctcttctccagtggagtgcctc
ggtcttcttcgtacaggaactctgaccactctgatacaaaggcgcgcgtccaggccagcacaaaggaggc
tatgtgggaggggtagcgatcgttgtcaaccagggggtccaccttttccaaagtatgcaaacacatgtca
ccctcttcaacatccaggaatgtgattggcttgtaggtgtatttcacgtgacctggggtccccgctgggg
gggtataaaagggggcggttctttgctcttcctcactgtcttccggatcgctgtccaggaacgtcagctg
ttggggtaggtattccctctcgaaggcgggcatgacctctgcactcaggttgtcagtttctaagaacgag
gaggatttgatattgacagtgccggttgagatgcctttcatgaggttttcgtccatttggtcagaaaaca
caatttttttattgtcaagtttggtggcaaatgatccatacagggcgttggataaaagtttggcaatgga
tcgcatggtttggttcttttccttgtccgcgcgctctttggcggcgatgttgagttggacatactcgcgt
gccaggcacttccattcggggaagatagttgttaattcatctggcacgattctcacttgccaccctcgat
tatgcaaggtaattaaatccacactggtggccacctcgcctcgaaggggttcattggtccaacagagcct
acctcctttcctagaacagaaagggggaagtgggtctagcataagttcatcgggagggtctgcatccatg
gtaaagattcccggaagtaaatccttatcaaaatagctgatgggagtggggtcatctaaggccatttgcc
attctcgagctgccagtgcgcgctcatatgggttaaggggactgccccagggcatgggatgggtgagagc
agaggcatacatgccacagatgtcatagacgtagatgggatcctcaaagatgcctatgtaggttggatag
catcgcccccctctgatacttgctcgcacatagtcatatagttcatgtgatggcgctagcagccccggac
ccaagttggtgcgattgggtttttctgttctgtagacgatctggcgaaagatggcgtgagaattggaaga
gatggtgggtctttgaaaaatgttgaaatgggcatgaggtagacctacagagtctctgacaaagtgggca
taagattcttgaagcttggttaccagttcggcggtgacaagtacgtctagggcgcagtagtcaagtgttt
cttgaatgatgtcataacctggttggtttttcttttcccacagttcgcggttgagaaggtattcttcgcg
atccttccagtactcttctagcggaaacccgtctttgtctgcacggtaagatcctagcatgtagaactga
ttaactgccttgtaagggcagcagcccttctctacgggtagagagtatgcttgagcagcttttcgtagcg
aagcgtgagtaagggcaaaggtgtctctgaccatgactttgagaaattggtatttgaagtccatgtcgtc
acaggctccctgttcccagagttggaagtctacccgtttcttgtaggcggggttgggcaaagcgaaagta
acatcattgaagagaatcttaccggctctgggcataaaattgcgagtgatgcggaaaggctgtggtactt
ccgctcgattgttgatcacctgggcagctaggacgatttcgtcgaaaccgttgatgttgtgtcctacgat
gtataattctatgaaacgcggcgtgcctctgacgtgaggtagcttactgagctcatcaaaggttaggtct
gtggggtcagataaggcgtagtgttcgagagcccattcgtgcaggtgaggatttgcatgtaggaatgatg
accaaagatctaccgccagtgctgtttgtaactggtcccgatactgacgaaaatgccggccaattgccat
tttttctggagtgacacagtagaaggttctggggtcttgttgccatcgatcccacttgagtttaatggct
agatcgtgggccatgttgacgagacgctcttctcctgagagtttcatgaccagcatgaaaggaactagtt
gtttgccaaaggatcccatccaggtgtaagtttccacatcgtaggtcaggaagagtctttctgtgcgagg
atgagagccgatcgggaagaactggatttcctgccaccagttggaggattggctgttgatgtgatggaag
tagaagtttctgcggcgcgccgagcattcgtgtttgtgcttgtacagacggccgcagtagtcgcagcgtt
gcacgggttgtatctcgtgaatgagctgtacctggcttcccttgacgagaaatttcagtgggaagccgag
gcctggcgattgtatctcgtgctcttctatattcgctgtatcggcctgttcatcttctgtttcgatggtg
gtcatgctgacgagcccccgcgggaggcaagtccagacctcggcgcgggaggggcggagctgaaggacga
gagcgcgcaggctggagctgtccagagtcctgagacgctgcggactcaggttagtaggtagggacagaag
attaacttgcatgatcttttccagggcgtgcgggaggttcagatggtacttgatttccacaggttcgttt
gtagagacgtcaatggcttgcagggttccgtgtcctttgggcgccactaccgtacctttgttttttcttt
tgatcggtggtggctctcttgcttcttgcatgctcagaagcggtgacggggacgcgcgccgggcggcagc
ggttgttccggacccgggggcatggctggtagtggcacgtcggcgccgcgcacgggcaggttctggtatt
gcgctctgagaagacttgcgtgcgccaccacgcgtcgattgacgtcttgtatctgacgtctctgggtgaa
agctaccggccccgtgagcttgaacctgaaagagagttcaacagaatcaatttcggtatcgttaacggca
gcttgtctcagtatttcttgtacgtcaccagagttgtcctggtaggcgatctccgccatgaactgctcga
tttcttcctcctgaagatctccgcgacccgctctttcgacggtggccgcgaggtcattggagatacggcc
catgagttgggagaatgcattcatgcccgcctcgttccagacgcggctgtaaaccacggccccctcggag
tctcttgcgcgcatcaccacctgagcgaggttaagctccacgtgtctggttaagaccgcatagttgcata
ggcgctgaaaaaggtagttgagtgtggtggcaatgtgttcggcgacgaagaaatacatgatccatcgtct
cagcggcatttcgctaacatcgcccagagcttccaagcgctccatggcctcgtagaagtccacggcaaaa
ttaaaaaactgggagtttcgcgcggacacggtcaattcctcctcgagaagacggatgagttcggctatgg
tggcccgtacttcgcgttcgaaggctcccgggatctcttcttcctcttctatctcttcttccactaacat
ctcttcttcgtcttcaggcgggggcggagggggcacgcggcgacgtcgacggcgcacgggcaaacggtcg
atgaatcgttcaatgacctctccgcggcggcggcgcatggtttcagtgacggcgcggccgttctcgcgcg
gtcgcagagtaaaaacaccgccgcgcatctccttaaagtggtgactgggaggttctccgtttgggaggga
gagggcgctgattatacattttattaattggcccgtagggactgcgcgcagagatctgatcgtgtcaaga
tccacgggatctgaaaacctttcgacgaaagcgtctaaccagtcacagtcacaaggtaggctgagtacgg
cttcttgtgggcgggggtggttatgtgttcggtctgggtcttctgtttcttcttcatctcgggaaggtga
gacgatgctgctggtgatgaaattaaagtaggcagttctaagacggcggatggtggcgaggagcaccagg
tctttgggtccggcttgctggatacgcaggcgattggccattccccaagcattatcctgacatctagcaa
gatctttgtagtagtcttgcatgagccgttctacgggcacttcttcctcacccgttctgccatgcatacg
tgtgagtccaaatccgcgcattggttgtaccagtgccaagtcagctacgactctttcggcgaggatggct
tgctgtacttgggtaagggtggcttgaaagtcatcaaaatccacaaagcggtggtaagcccctgtattaa
tggtgtaagcacagttggccatgactgaccagttaactgtctggtgaccagggcgcacgagctcggtgta
tttaaggcgcgaataggcgcgggtgtcaaagatgtaatcgttgcaggtgcgcaccagatactggtaccct
ataagaaaatgcggcggtggttggcggtagagaggccatcgttctgtagctggagcgccaggggcgaggt
cttccaacataaggcggtgatagccgtagatgtacctggacatccaggtgattcctgcggcggtagtaga
agcccgaggaaactcgcgtacgcggttccaaatgttgcgtagcggcatgaagtagttcattgtaggcacg
gtttgaccagtgaggcgcgcgcagtcattgatgctctatagacacggagaaaatgaaagcgttcagcgac
tcgactccgtagcctggaggaacgtgaacgggttgggtcgcggtgtaccccggttcgagacttgtactcg
agccggccggagccgcggctaacgtggtattggcactcccgtctcgacccagcctacaaaaatccaggat
acggaatcgagtcgttttgctggtttccgaatggcagggaagtgagtcctatttttttttttttttgccg
ctcagaatgcatcccgtgctgcgacagatgcgcccccaacaacagcccccctcgcagcagcagcagcagc
aaccacaaaaggctgtccctgcaactactgcaactgccgccgtgagcggtgcgggacagcccgcctatga
tctggacttggaagagggcgaaggactggcacgtctaggtgcgccttcgcccgagcggcatccgcgagtt
caactgaaaaaagattctcgcgaggcgtatgtgccccaacagaacctatttagagacagaagcggcgagg
agccggaggagatgcgagcttcccgctttaacgcgggtcgtgagctgcgtcacggtttggaccgaagacg
agtgttgcgagacgaggatttcgaagttgatgaagtgacagggatcagtcctgccagggcacacgtggct
gcagccaaccttgtatcggcttacgagcagacagtaaaggaagagcgtaacttccaaaagtcttttaata
atcatgtgcgaaccctgattgcccgcgaagaagttacccttggtttgatgcatttgtgggatttgatgga
agctatcattcagaaccctactagcaaacctctgaccgcccagctgtttctggtggtgcaacacagcaga
gacaatgaggctttcagagaggcgctgctgaacatcaccgaacccgaggggagatggttgtatgatctta
tcaacattctacagagtatcatagtgcaggagcggagcctgggcctggccgagaaggtagctgccatcaa
ttactcggttttgagcttgggaaaatattacgctcgcaaaatctacaagactccatacgttcccatagac
aaggaggtgaagatagatgggttctacatgcgcatgacgctcaaggtcttgaccctgagcgatgatcttg
gggtgtatcgcaatgacagaatgcatcgcgcggttagcgccagcaggaggcgcgagttaagcgacaggga
actgatgcacagtttgcaaagagctctgactggagctggaaccgagggtgagaattacttcgacatggga
gctgacttgcagtggcagcctaatcgcagggctctgagcgccgcgacggcaggatgtgagcttccttaca
tagaagaggcggatgaaggcgaggaggaagagggcgagtacttggaagactgatggcacaacccgtgttt
tttgetagatggaacagcaagcaccggatcccgcaatgcgggcggcgctgcagagccagecgtccggcat
taactcctcggacgattggacccaggccatgcaacgtatcatggcgttgacgactcgcaaccccgaagcc
tttagacagcaaccccaggccaaccgtctatcggccatcatggaagctgtagtgccttcccgatctaatc
ccactcatgagaaggtcctggccatcgtgaacgcgttggtggagaacaaagctattcgtccagatgaggc
cggactggtatacaacgctctcttagaacgcgtggctcgctacaacagtagcaatgtgcaaaccaatttg
gaccgtatgataacagatgtacgcgaagccgtgtctcagcgcgaaaggttccagcgtgatgccaacctgg
gttcgctggtggcgttaaatgctttcttgagtactcagcctgctaatgtgccgcgtggtcaacaggatta
tactaactttttaagtgctttgagactgatggtatcagaagtacctcagagcgaagtgtatcagtccggt
cotgattacttctttcagactagcagacagggcttgcagacggtaaatctgagecaagcttttaaaaacc
ttaaaggtttgtggggagtgcatgccccggtaggagaaagagcaaccgtgtctagcttgttaactccgaa
ctcccgcctgttattactgttggtagctcctttcaccgacagcggtagcatcgaccgtaattcctatttg
ggttacctactaaacctgtatcgcgaagccatagggcaaagtcaggtggacgagcagacctatcaagaaa
ttacccaagtcagtcgcgctttgggacaggaagacactggcagtttggaagccactctgaacttcttgct
taccaatcggtctcaaaagatccctcctcaatatgctcttactgcggaggaggagaggatccttagatat
gtgcagcagagcgtgggattgtttctgatgcaagagggggcaactccgactgcagcactggacatgacag
cgcgaaatatggagcccagcatgtatgccagtaaccgacctttcattaacaaactgctggactacttgca
cagagctgccgctatgaactctgattatttcaccaatgccatcttaaacccgcactggctgcccccacct
ggtttctacacgggcgaatatgacatgcccgaccctaatgacggatttctgtgggacgacgtggacagcg
atgttttttcacctctttctgatcatcgcacgtggaaaaaggaaggcggtgatagaatgcattcttctgc
ategetgtccggggtcatgggtgetaccgcggctgageccgagtctgcaagtccttttcctagtctaccc
ttttctctacacagtgtacgtagcagcgaagtgggtagaataagtcgcccgagtttaatgggcgaagagg
agtacctaaacgattccttgctcagaccggcaagagaaaaaaatttcccaaacaatggaatagaaagttt
ggtggataaaatgagtagatggaagacttatgctcaggatcacagagacgagcctgggatcatggggact
acaagtagagcgagccgtagacgccagcgccatgacagacagaggggtcttgtgtgggacgatgaggatt
cggccgatgatagcagcgtgttggacttgggtgggagaggaaggggcaacccgtttgctcatttgcgccc
tegettgggtggtatgttgtgaaaaaaaataaaaaagaaaaactcaccaaggccatggcgacgagcgtac
gttcgttcttctttattatctgtgtctagtataatgaggcgagtcgtgctaggcggagcggtggtgtatc
cggagggtcctcctccttcgtacgagagcgtgatgcagcagcagcaggcgacggcggtgatgcaatcccc
actggaggctccctttgtgcctccgcgatacctggcacctacggagggcagaaacagcattcgttactcg
gaactggcacctcagtacgataccaccaggttgtatctggtggacaacaagtcggcggacattgcttctc
tgaactatcagaatgaccacagcaacttcttgaccacggtggtgcagaacaatgactttacccctacgga
agccagcacccagaccattaactttgatgaacgatcgcggtggggcggtcagctaaagaccatcatgcat
actaacatgccaaacgtgaacgagtatatgtttagtaacaagttcaaagcgcgtgtgatggtgtccagaa
aacctcccgacggtgctgcagttggggatacttatgatcacaagcaggatattttggaatatgagtggtt
cgagtttactttgccagaaggcaacttttcagttactatgactattgatttgatgaacaatgccatcata
gataattacttgaaagtgggtagacagaatggagtgcttgaaagtgacattggtgttaagttcgacacca
ggaacttcaagctgggatgggatcccgaaaccaagttgatcatgcctggagtgtatacgtatgaagcctt
ccatcctgacattgtcttactgcctggctgcggagtggattttaccgagagtcgtttgagcaaccttctt
ggtatcagaaaaaaacagccatttcaagagggttttaagattttgtatgaagatttagaaggtggtaata
ttccggccctcttggatgtagatgcctatgagaacagtaagaaagaacaaaaagccaaaatagaagctgc
tacagctgctgcagaagctaaggcaaacatagttgccagcgactctacaagggttgctaacgctggagag
gtcagaggagacaattttgcgccaacacctgttccgactgcagaatcattattggccgatgtgtctgaag
gaacggacgtgaaactcactattcaacctgtagaaaaagatagtaagaatagaagctataatgtgttgga
agacaaaatcaacacagcctatcgcagttggtatctttcgtacaattatggcgatcccgaaaaaggagtg
cgttcctggacattgctcaccacctcagatgtcacctgcggagcagagcaggtttactggtcgcttccag
acatgatgaaggatcctgtcactttccgctccactagacaagtcagtaactaccctgtggtgggtgcaga
gcttatgcccgtcttctcaaagagcttctacaacgaacaagctgtgtactcccagcagctccgccagtcc
acctcgcttacgcacgtcttcaaccgctttcctgagaaccagattttaatccgtccgccggcgcccacca
ttaccaccgtcagtgaaaacgttcctgctctcacagatcacgggaccctgccgttgcgcagcagtatccg
gggagtccaacgtgtgaccgttactgacgccagacgccgcacctgtccctacgtgtacaaggcactgggc
atagtcgcaccgcgcgtcctttcaagccgcactttctaaaaaaaaaatgtccattcttatctcgcccagt
aataacaccggttggggtctgcgcgctccaagcaagatgtacggaggcgcacgcaaacgttctacccaac
atcccgtgcgtgttcgcggacattttcgcgctccatggggtgccctcaagggccgcactcgcgttcgaac
caccgtcgatgatgtaatcgatcaggtggttgccgacgcccgtaattatactcctactgcgcctacatct
actgtggatgcagttattgacagtgtagtggctgacgctcgcaactatgctcgacgtaagagccggcgaa
ggcgcattgccagacgccaccgagctaccactgccatgcgagccgcaagagctctgctacgaagagctag
acgcgtggggcgaagagccatgcttagggcggccagacgtgcagcttcgggcgccagcgccggcaggtcc
cgcaggcaagcagccgctgtcgcagcggcgactattgccgacatggcccaatcgcgaagaggcaatgtat
actgggtgcgtgacgctgccaccggtcaacgtgtacccgtgcgcacccgtccccctcgcacttagaagat
actgagcagtctccgatgttgtgtcccagcggcgaggatgtccaagcgcaaatacaaggaagaaatgctg
caggttatcgcacctgaagtctacggccaaccgttgaaggatgaaaaaaaaccccgcaaaatcaagcggg
ttaaaaaggacaaaaaagaagaggaagatggcgatgatgggctggcggagtttgtgcgcgagtttgcccc
acggcgacgcgtgcaatggcgtgggcgcaaagttcgacatgtgttgagacctggaacttcggtggtcttt
acacccggcgagcgttcaagcgctacttttaagcgttcctatgatgaggtgtacggggatgatgatattc
ttgagcaggcggctgaccgattaggcgagtttgcttatggcaagcgtagtagaataacttccaaggatga
gacagtgtcaatacccttggatcatggaaatcccacccctagtcttaaaccggtcactttgcagcaagtg
ttacccgtaactccgcgaacaggtgttaaacgcgaaggtgaagatttgtatcccactatgcaactgatgg
tacccaaacgccagaagttggaggacgttttggagaaagtaaaagtggatccagatattcaacctgaggt
taaagtgagacccattaagcaggtagcgcctggtctgggggtacaaactgtagacattaagattcccact
gaaagtatggaagtgcaaactgaacccgcaaagcctactgccacctccactgaagtgcaaacggatccat
ggatgcccatgcctattacaactgacgccgccggtcccactcgaagatcccgacgaaagtacggtccagc
aagtctgttgatgcccaattatgttgtacacccatctattattcctactcctggttaccgaggcactcgc
tactatcgcagccgaaacagtacctcccgccgtcgccgcaagacacctgcaaatcgcagtcgtcgccgta
gacgcacaagcaaaccgactcccggcgccctggtgcggcaagtgtaccgcaatggtagtgcggaaccttt
gacactgccgcgtgcgcgttaccatccgagtatcatcacttaatcaatgttgccgctgcctccttgcaga
tatggccctcacttgtcgccttcgcgttcccatcactggttaccgaggaagaaactcgcgccgtagaaga
gggatgttgggacgcggaatgcgacgctacaggcgacggcgtgctatccgcaagcaattgcggggtggtt
ttttaccagccttaattccaattatcgctgctgcaattggcgcgataccaggcatagcttccgtggcggt
tcaggcctcgcaacgacattgacattggaaaaaaaacgtataaataaaaaaaaatacaatggactctgac
actcctggtcctgtgactatgttttcttagagatggaagacatcaatttttcatccttggctccgcgaca
cggcacgaagccgtacatgggcacctggagcgacatcggcacgagccaactgaacgggggcgccttcaat
tggagcagtatctggagcgggcttaaaaattttggctcaaccataaaaacatacgggaacaaagcttgga
acagcagtacaggacaggcgcttagaaataaacttaaagaccagaacttccaacaaaaagtagtcgatgg
gatagcttccggcatcaatggagtggtagatttggctaaccaggctgtgcagaaaaagataaacagtcgt
ttggacccgccgccagcaaccccaggtgaaatgcaagtggaggaagaaattcctccgccagaaaaacgag
gcgacaagcgtccgcgtcccgatttggaagagacgctggtgacgcgcgtagatgaaccgccttcttatga
ggaagcaacgaagcttggaatgcccaccactagaccgatagccccaatggccaccggggtgatgaaacct
tctcagttgcatcgacccgtcaccttggatttgccccctccccctgctgctactgctgtacccgcttcta
agcctgtcgctgccccgaaaccagtcgccgtagccaggtcacgtcccgggggcgctcctcgtccaaatgc
gcactggcaaaatactctgaacagcatcgtgggtctaggcgtgcaaagtgtaaaacgccgtcgctgcttt
taattaaatatggagtagcgcttaacttgcctatctgtgtatatgtgtcattacacgccgtcacagcagc
agaggaaaaaaggaagaggtcgtgcgtcgacgctgagttactttcaagatggccaccccatcgatgctgc
cccaatgggcatacatgcacatcgccggacaggatgcttcggagtacctgagtccgggtctggtgcagtt
cgcccgcgccacagacacctacttcaatctgggaaataagtttagaaatcccaccgtagcgccgacccac
gatgtgaccaccgaccgtagccagcggctcatgttgcgcttcgtgcccgttgaccgggaggacaatacat
actcttacaaagtgcggtacaccctggccgtgggcgacaacagagtgctggatatggccagcacgttctt
tgacattaggggcgtgttggacagaggtcccagtttcaaaccctattctggtacggcttacaactctctg
gctcctaaaggcgctccaaatgcatctcaatggattgcaaaaggcgtaccaactgcagcagccgcaggca
atggtgaagaagaacatgaaacagaggagaaaactgctacttacacttttgccaatgctcctgtaaaagc
cgaggctcaaattacaaaagagggcttaccaataggtttggagatttcagctgaaaacgaatctaaaccc
atctatgcagataaactttatcagccagaacctcaagtgggagatgaaacttggactgacctagacggaa
aaaccgaagagtatggaggcagggctctaaagcctactactaacatgaaaccctgttacgggtcctatgc
gaagcctactaatttaaaaggtggtcaggcaaaaccgaaaaactcggaaccgtcgagtgaaaaaattgaa
tatgatattgacatggaattttttgataactcatcgcaaagaacaaacttcagtcctaaaattgtcatgt
atgcagaaaatgtaggtttggaaacgccagacactcatgtagtgtacaaacctggaacagaagacacaag
ttccgaagctaatttgggacaacagtctatgcccaacagacccaactacattggcttcagagataacttt
attggactcatgtactataacagtactggtaacatgggggtgctggctggtcaagcgtctcagttaaatg
cagtggttgacttgcaggacagaaacacagaactttcttaccaactcttgcttgactctctgggcgacag
aaccagatactttagcatgtggaatcaggctgtggacagttatgatcctgatgtacgtgttattgaaaat
catggtgtggaagatgaacttcccaactattgttttccactggacggcataggtgttccaacaaccagtt
acaaatcaatagttccaaatggagaagataataataattggaaagaacctgaagtaaatggaacaagtga
gatcggacagggtaatttgtttgccatggaaattaaccttcaagccaatctatggcgaagtttcctttat
tccaatgtggctctgtatctcccagactcgtacaaatacaccccgtccaatgtcactcttccagaaaaca
aaaacacctacgactacatgaacgggcgggtggtgccgccatctctagtagacacctatgtgaacattgg
tgccaggtggtctctggatgccatggacaatgtcaacccattcaaccaccaccgtaacgctggcttgcgt
taccgatctatgcttctgggtaacggacgttatgtgcctttccacatacaagtgcctcaaaaattcttcg
ctgttaaaaacctgctgcttctcccaggctcctacacttatgagtggaactttaggaaggatgtgaacat
ggttctacagagttccctcggtaacgacctgcgggtagatggcgccagcatcagtttcacgagcatcaac
ctctatgctacttttttccccatggctcacaacaccgcttccacccttgaagccatgctgcggaatgaca
ccaatgatcagtcattcaacgactacctatctgcagctaacatgctctaccccattcctgccaatgcaac
caatattcccatttccattccttctcgcaactgggcggctttcagaggctggtcatttaccagactgaaa
accaaagaaactccctctttggggtctggatttgacccctactttgtctattctggttctattccctacc
tggatggtaccttctacctgaaccacacttttaagaaggtttccatcatgtttgactcttcagtgagctg
gcctggaaatgacaggttactatctcctaacgaatttgaaataaagcgcactgtggatggcgaaggctac
aacgtagcccaatgcaacatgaccaaagactggttcttggtacagatgctcgccaactacaacatcggct
atcagggcttctacattccagaaggatacaaagatcgcatgtattcatttttcagaaacttccagcccat
gagcaggcaggtggttgatgaggtcaattacaaagacttcaaggccgtcgccataccctaccaacacaac
aactctggctttgtgggttacatggctccgaccatgcgccaaggtcaaccctatcccgctaactatccct
atccactcattggaacaactgccgtaaatagtgttacgcagaaaaagttcttgtgtgacagaaccatgtg
gcgcataccgttctcgagcaacttcatgtctatgggggcccttacagacttgggacagaatatgctctat
gccaactcagctcatgctctggacatgacctttgaggtggatcccatggatgagcccaccctgctttatc
ttctcttcgaagttttcgacgtggtcagagtgcatcagccacaccgcggcatcatcgaggcagtctacct
gcgtacaccgttctcggccggtaacgctaccacgtaagaagcttcttgcttcttgcaaatagcagctgca
accatggcctgcggatcccaaaacggctccagcgagcaagagctcagagccattgtccaagacctgggtt
gcggaccctattttttgggaacctacgataagcgcttcccggggttcatggcccccgataagctcgcctg
tgccattgtaaatacggccggacgtgagacggggggagagcactggttggctttcggttggaacccacgt
tctaacacctgctacctttttgatccttttggattctcggatgatcgtctcaaacagatttaccagtttg
aatatgagggtctcctgcgccgcagcgctcttgctaccaaggaccgctgtattacgctggaaaaatctac
ccagaccgtgcagggcccccgttctgccgcctgcggacttttctgctgcatgttccttcacgcctttgtg
cactggcctgaccgtcccatggacggaaaccccaccatgaaattgctaactggagtgccaaacaacatgc
ttcattctcctaaagtccagcccaccctgtgtgacaatcaaaaagcactctaccattttcttaataccca
ttcgccttattttcgctctcatcgtacacacatcgaaagggccactgcgttcgaccgtatggatgttcaa
taatgactcatgtaaacaacgtgttcaataaacatcactttatttttttacatgtatcaaggctctggat
tacttatttatttacaagtcgaatgggttctgacgagaatcagaatgacccgcaggcagtgatacgttgc
ggaactgatacttgggttgccacttgaattcgggaatcaccaacttgggaaccggtatatcgggcaggat
gtcactccacagctttctggtcagctgcaaagctccaagcaggtcaggagccgaaatcttgaaatcacaa
ttaggaccagtgctctgagcgcgagagttgcggtacaccggattgcagcactgaaacaccatcagcgacg
gatgtctcacgcttgccagcacggtgggatctgcaatcatgcccacatccagatcttcagcattggcaat
gctgaacggggtcatcttgcaggtctgcctacccatggcgggcacccaattaggcttgtggttgcaatcg
cagtgcagggggatcagtatcatcttggcctgatcctgtctgattcctggatacacggctctcatgaaag
catcatattgcttgaaagcctgctgggctttactaccctcgggataaaacatcccgcaggacctgctcga
aaactggttagcctgcacagccggcatcattcacacagcagcgggcgtcattgttggctatttgcaccac
acttctgccccagcggttttgggtgattttggttcgctcgggattctcctttaaggctcgttgtccgttc
tcgctggccacatccatctcgataatctgctccttctgaatcataatattgccatgcaggcacttcagct
tgccctcataatcattgcagccatgaggccacaacgcacagcctgtacattcccaattatggtgggcgat
ctgagaaaaagaatgtatcattccctgcagaaatcttcccatcatcgtgctcagtgtcttgtgactagtg
aaagttaactggatgcctcggtgctcttcgtttacgtactggtgacagatgcgcttgtattgttcgtgtt
gctcaggcattagtttaaaacaggttctaagttcgttatccagcctgtacttctccatcagcagacacat
cacttccatgcctttctcccaagcagacaccaggggcaagctaatcggattcttaacagtgcaggcagca
gctcctttagccagagggtcatctttagcgatcttctcaatgcttcttttgccatccttctcaacgatgc
gcacgggcgggtagctgaaacccactgctacaagttgcgcctcttctctttcttcttcgctgtcttgact
gatgtcttgcatggggatatgtttggtcttccttggcttctttttggggggtatcggaggaggaggactg
tcgctccgttccggagacagggaggattgtgacgtttcgctcaccattaccaactgactgtcggtagaag
aacctgaccccacacggcgacaggtgtttttcttcgggggcagaggtggaggcgattgcgaagggctgcg
gtccgacctggaaggcggatgactggcagaaccccttccgcgttcgggggtgtgctccctgtggcggtcg
cttaactgatttccttcgcggctggccattgtgttctcctaggcagagaaacaacagacatggaaactca
gccattgctgtcaacatcgccacgagtgccatcacatctcgtcctcagcgacgaggaaaaggagcagagc
ttaagcattccaccgcccagtcctgccaccacctctaccctagaagataaggaggtcgacgcatctcatg
acatgcagaataaaaaagcgaaagagtctgagacagacatcgagcaagacccgggctatgtgacaccggt
ggaacacgaggaagagttgaaacgctttctagagagagaggatgaaaactgcccaaaacagcgagcagat
aactatcaccaagatgctggaaatagggatcagaacaccgactacctcatagggcttgacggggaagacg
cgctccttaaacatctagcaagacagtcgctcatagtcaaggatgcattattggacagaactgaagtgcc
catcagtgtggaagagctcagctgcgcctacgagcttaaccttttttcacctcgtactccccccaaacgt
cagccaaacggcacctgcgagccaaatcctcgcttaaacttttatccagcttttgctgtgccagaagtac
tggctacctatcacatcttttttaaaaatcaaaaaattccagtctcctgccgcgctaatcgcacccgcgc
cgatgccctactcaatctgggacctggttcacgcttacctgatatagcttccttggaagaggttccaaag
atcttcgagggtctgggcaataatgagactcgggccgcaaatgctctgcaaaagggagaaaatggcatgg
atgagcatcacagcgttctggtggaattggaaggcgataatgccagactcgcagtactcaagcgaagcgt
cgaggtcacacacttcgcatatcccgctgtcaacctgccccctaaagtcatgacggcggtcatggaccag
ttactcattaagcgcgcaagtcccctttcagaagacatgcatgacccagatgcctgtgatgagggtaaac
cagtggtcagtgatgagcagctaacccgatggctgggcaccgactctccccgggatttggaagagcgtcg
caagcttatgatggccgtggtgctggttaccgtagaactagagtgtctccgacgtttctttaccgattca
gaaaccttgcgcaaactcgaagagaatctgcactacacttttagacacggctttgtgcggcaggcatgca
agatatctaacgtggaactcaccaacctggtttcctacatgggtattctgcatgagaatcgcctaggaca
aagcgtgctgcacagcacccttaagggggaagcccgccgtgattacatccgcgattgtgtctatctctac
ctgtgccacacgtggcaaaccggcatgggtgtatggcagcaatgtttagaagaacagaacttgaaagagc
ttgacaagctcttacagaaatctcttaaggttctgtggacagggttcgacgagcgcaccgtcgcttccga
cctggcagacctcatcttcccagagcgtctcagggttactttgcgaaacggattgcctgactttatgagc
cagagcatgcttaacaattttcgctctttcatcctggaacgctccggtatcctgcccgccacctgctgcg
cactgccctccgactttgtgcctctcacctaccgcgagtgccccccgccgctatggagtcactgctacct
gttccgtctggccaactatctctcctaccactcggatgtgatcgaggatgtgagcggagacggcttgctg
gagtgccactgccgctgcaatctgtgcacgccccaccggtccctagcttgcaacccccagttgatgagcg
aaacccagataataggcacctttgaattgcaaggccccagcagccaaggcgatgggtcttctcctgggca
aagtttaaaactgaccccgggactgtggacctccgcctacttgcgcaagtttgctccggaagattaccac
ccctatgaaatcaagttctatgaggaccaatcacagcctccaaaggccgaactttcggcttgcgtcatca
cccagggggcaattctggcccaattgcaagccatccaaaaatcccgccaagaatttctactgaaaaaggg
taagggggtctaccttgacccccagaccggcgaggaactcaacacaaggttccctcaggatgtcccaacg
acgagaaaacaagaagttgaaggtgcagccgccgcccccagaagatatggaggaagattgggacagtcag
gcagaggaggcggaggaggacagtctggaggacagtctggaggaagacagtttggaggaggaaaacgagg
aggcagaggaggtggaagaagtaaccgccgacaaacagttatcctcggctgcggagacaagcaacagcgc
taccatctccgctccgagtcgaggaacccggcggcgtcccagcagtagatgggacgagaccggacgcttc
ccgaacccaaccagcgcttccaagaccggtaagaaggatcggcagggatacaagtcctggcgggggcata
agaatgccatcatctcctgcttgcatgagtgcgggggcaacatatccttcacgcggcgctacttgctatt
ccaccatggggtgaactttccgcgcaatgttttgcattactaccgtcacctccacagcccctactatagc
cagcaaatcccgacagtctcgacagataaagacagcggcggcgacctccaacagaaaaccagcagcggca
gttagaaaatacacaacaagtgcagcaacaggaggattaaagattacagccaacgagccagcgcaaaccc
gagagttaagaaatcggatctttccaaccctgtatgccatcttccagcagagtcggggtcaagagcagga
actgaaaataaaaaaccgatctctgcgttcgctcaccagaagttgtttgtatcacaagagcgaagatcaa
cttcagcgcactctcgaggacgccgaggctctcttcaacaagtactgcgcgctgactcttaaagagtagg
cagcgaccgcgcttattcaaaaaaggcgggaattacatcatcctcgacatgagtaaagaaattcccacgc
cttacatgtggagttatcaaccccaaatgggattggcagcaggcgcctcccaggactactccacccgcat
gaattggctcagcgccgggccttctatgatttctcgagttaatgatatacgcgcctaccgaaaccaaata
cttttggaacagtcagctcttaccaccacgccccgccaacaccttaatcccagaaattggcccgccgccc
tagtgtaccaggaaagtcccgctcccaccactgtattacttcctcgagacgcccaggccgaagtccaaat
gactaatgcaggtgcgcagttagctggcggctccaccctatgtcgtcacaggcctcggcataatataaaa
cgcctgatgatcagaggccgaggtatccagctcaacgacgagtcggtgagctctccgcttggtctacgac
cagacggaatctttcagattgccggctgcgggagatcttccttcacccctcgtcaggctgttctgacttt
ggaaagttcgtcttcgcaaccccgctcgggcggaatcgggaccgttcaatttgtagaggagtttactccc
tctgtctacttcaaccccttctccggatctcctgggcactacccggacgagttcataccgaacttcgacg
cgattagcgagtcagtggacggctacgattgatgtctggtgacgcggctgagctatctcggctgcgacat
ctagaccactgccgccgctttcgctgctttgcccgggaacttattgagttcatctacttcgaactcccca
aggatcaccctcaaggtccggcccacggagtgcggattactatcgaaggcaaaatagactctcgcctgca
acgaattttctcccagcggcccgtgctgatcgagcgagaccagggaaacaccacggttagtaatcaatta
cggggtcattagttcatagcccatatatggagttgcgatcgctgcgggccatgtcatacaccgccttcag
agcagccggacctatctgcccgttcgtgccgtcgttgttaatcaccacatggttattctgctcaaacgtc
ccggacgcctgcgaccggctgtctgccatgctgcccggtgtaccgacataaccgccggtggcatagccgc
gcatcagccggtaaagattccccacgccaatccggctggttgcctccttcgtgaagacaaactcaccacg
gtgaacaatccccgctggctcatatttgccgccggttcccgtaaatcctccggttgcaaaatggaatttc
gccgcagcggcctgaatggctgtaccgcctgacgcggatgcgccgccaccaacagccccgccaatggcgc
tgccgatactcccgacaatccccaccattgcctgcttaagcagaatttctgtcatcatggacagcacgga
acgggtgaagctgcgccagttctgctcactgccggtcagcatcgccgccatattctgtgcaataccatca
aaggtctgcgtggctgcactttttacctgcgacatactgtccgtggcgctctcttcccactcactccagc
cggacttcaggcctgccatccagttcccgcgaagctggtcttcagccgcccaggtctttttctgctctga
catgacgttattcagcgccagcggattatcgccatactgttccttcaggcgctgttccgtggcttcccgt
tctgcctgccggtcagtcagcccccggcttttcgcatcaatggcggcccgttttgcccgttgctgctgtg
cgaatttatccgcctgctgcgccagcgcgttcaggcgctcctgatacgtaaccttgtcgccaagtgcagc
cagctggcgtttgtactccagcgtctcatctttatgcgccagcagggatttctcctgtgcagacagctgg
cgacgttgcgccgcctcctccagtaccgcgaactgactctccgccttccacaaatcccggcgctgctggc
tgattttctcatttgctccggcatgcttctccagcgtccggagttctgcctgaagcgtcagcagggcagc
atgagcactgtcttcctgacgatcgcccgcagacaccttcacgctggactgtttcggctttttcagcgtc
gcttcataatcctttttcgccgccgccatcagcgtgttgtaatccgcctgcaggattttcccgtctttca
gtgccttgttcagttcttcctgacgggcggtatatttctccagcggcgtctgcagccgttcgtaagcctt
ctgcgcctcttcggtatatttcagccgtgacgcttcggtatcgctctgctgctgcgcatttttgtcctgt
tgagtctgctgctcagccttctttcgggcggcttcaagcgcaagacgggccttttcacgatcatcccagt
aacgcgcccgcgcttcatcgttaacaaaataatcatccttgcgcagattccagatgtcgtctgctttctt
atacgcagcctctgccttaatcagcatctcctgcgcggtatcaggacgaccaatatccagcaccgcatcc
cacatggatttgaatgcccgcgcagtcctgtctgcccaggtctccagcgtgcccatgttctctttcaggc
ggcgggtctggtcatcaaaccctttcgttgcggcctcgttcgccgcctgcaatgccccggcttcatcgcc
ggaacgctgcaactgagcaacatacgcaatctgctccgccgacacgttatggaactggcgagccatcgcc
gtcagccccgacgtcgggtctgtggtcagcttcccgaaggcttcagcgaccttgtccacctccacgccgg
atgcagaggagaaacgcgccacactctggctgatggacgcaatctgagcctcaccgcttacccccgcctt
aaccagtgcgctgagtgactcgctggtctggttaaacgtcagccctgccgcctgcccggctctggacagg
accagcatacgatctgccgtcagtcccgcctgattgccggaaaggaccagcgttttgttgaaatcggaca
gggttgagttgccctgataccaggcatacgccagcgcaccggtcgccaccgccagcgaggtggcccccac
catcggcagggtgatcgcaccggcaagccccctgaacatggggatcatcccgccgaaggagtccttcacc
tgccccccctgttgcagcaggatcagccacggactttgcccgcctgcaagctgcgtggccacgtcggtga
actgtgcaggcagcatacgcatggcggctttatactgcccgacggaaatccccgctttctgtgcagccag
cgcctgtcggctcagcgactgttcaacgactgccgctgtttttttcgcatcactttccgtaccagaaaaa
tgacgcctgactctggccatctgctcgtcaaatctggccgcatccagactcaaatcaacgacgtcgacta
agctctagcatttgtgaaccatcaccctaatcaagttttttggggtcgaggtgccgtaaagcactaaatc
ggaaccctaaagggagcccccgatttagagcttgacggggaaagccggcgaacgtggcgagaaaggaagg
gaagaaagcgaaaggagcgggcgctagggcgctggcaagtgtagcggtcacgctgcgcgtaaccaccaca
cccgccgcgcttaatgcgccgctacagggcgcgtggggataccccctagagccccagctggttctttccg
cctcagaagccatagagcccaccgcatccccagcatgcctgctattgtcttcccaatcctcccccttgct
gtcctgccccaccccaccccccagaatagaatgacacctactcagacaatgcgatgcaatttcctcattt
tattaggaaaggacagtgggagtggcaccttccagggtcaaggaaggcacgggggaggggcaaacaacag
atggctggcaactagaaggcacagtcgaggctgatcagcgggtttgctagcttaggcgaaggcgatgggg
gtcttgaaggcgtgctggtactccacgatgcccagctcggtgttgctgtgcagctcctccacgcggcgga
aggcgaacatggggcccccgttctgcaggatgctggggtggatggcgctcttgaagtgcatgtggctgtc
caccacgaagctgtagtagccgccgtcgcgcaggctgaaggtgcgggcgaagctgcccaccagcacgtta
tcgcccatggggtgcaggtgctccacggtggcgttgctgcggatgatcttgtcggtgaagatcacgctgt
cctcggggaagccggtgcccaccaccttgaagtcgccgatcacgcggccggcctcgtagcggtagctgaa
gctcacgtgcagcacgccgccgtcctcgtacttctcgatgcgggtgttggtgtagccgccgttgttgatg
gcgtgcaggaaggggttctcgtagccgctggggtaggtgccgaagtggtagaagccgtagcccatcacgt
ggctcagcaggtaggggctgaaggtcagggcgcctttggtgctcttcatcttgttggtcatgcggccctg
ctcgggggtgccctctccgccgcccaccagctcgaactccacgccgttcagggtgccggtgatgcggcac
tcgatcttcatggcgggcatggtggctagcctagccagcttgggtctccctatagtgagtcgtattaatt
tcgataagccagtaagcagtgggttctctagttagccagagagctctgcttatatagacctcccaccgta
cacgcctaccgcccatttgcgtcaatggggcggagttgttacgacattttggaaagtcccgttgattttg
gtgccaaaacaaactcccattgacgtcaatggggtggagacttggaaatccccgtgagtcaaaccgctat
ccacgcccattgatgtactgccaaaaccgcatcaccatggtaatagcgatgactaatacgtagatgtact
gccaagtaggaaagtcccataaggtcatgtactgggcataatgccaggcgggccatttaccgtcattgac
gtcaatagggggcgtacttggcatatgatacacttgatgtactgccaagtgggcagtttaccgtaaatac
tccacccattgacgtcaatggaaagtccctattggcgttactatgggaacatacgtcattattgacgtca
atgggcgggggtcgttgggcggtcagccaggcgggccatttaccgtaagttatgtaacgcggaactccat
atatgggctatgaactaatgaccccgtaattgattactattaataactacaataatcaatgtcaacgcgt
atatctggcccgtacatcgcgaagcagcgcaaaacgcctaaccctaagcagattcttcatgcaattaagc
ttcgcggtgcttcttcagtacgctacggcaaatgtcatcgacgtttttatccggaaactgctgtctggct
ttttttgatttcagaattagcctgacgggcaatgctgcgaagggcgttttcctgctgaggtgtcattgaa
caagtcccatgtcggcaagcataagcacacagaatatgaagcccgctgccagaaaaatgcattccgtggt
tgtcatacctggtttctctcatctgcttctgctttcgccaccatcatttccagcttttgtgaaagggatg
cggctaacgtatgaaattcttcgtctgtttctactggtattggcacaaacctgattccaatttgagcaag
gctatgtgccatctcgatactcgttcttaactcaacagaagatgctttgtgcatacagcccctcgtttat
tatttatctcctcagccagccgctgtgctttcagtggatttcggataacagaaaggccgggaaataccca
gcctcgctttgtaacggagtagacgaaagtgattgcgcctacccggatattatcgtgaggatgcgtcatc
gccattgctccccaaatacaaaaccaatttcagccagtgcctcgtccattttttcgatgaactccggcac
gatctcgtcaaaactcgccatgtacttttcatcccgctcaatcacgacataatgcaggccttcacgcttc
atacgcgggtcatagttggcaaagtaccaggcattttttcgcgtcacccacatgctgtactgcacctggg
ccatgtaagctgactttatggcctcgaaaccaccgagccggaacttcatgaaatcccgggaggtaaacgg
gcatttcagttcaaggccgttgccgtcactgcataaaccatcgggagagcaggcggtacgcatactttcg
tcgcgatagatgatcggggattcagtaacattcacgccggaagtgaattcaaacagggttctggcgtcgt
tctcgtactgttttccccaggccagtgctttagcgttaacttccggagccacaccggtgcaaacctcagc
aagcagggtgtggaagtaggacattttcatgtcaggccacttctttccggagcggggttttgctatcacg
ttgtgaacttctgaagcggtgatgacgccgagccgtaatttgtgccacgcatcatccccctgttcgacag
ctctcacatcgatcccggtacgctgcaggataatgtccggtgtcatgctgccaccttctgctctgcggct
ttctgtttcaggaatccaagagcttttactgcttcggcctgtgtcagttctgacgatgcacgaatgtcgc
ggcgaaatatctgggaacagagcggcaataagtcgtcatcccatgttttatccagggcgatcagcagagt
gttaatctcctgcatggtttcatcgttaaccggagtgatgtcgcgttccggctgacgttctgcagtgtat
gcagtattttcgacaatgcgctcggcttcatccttgtcatagataccagcaaatccgaagcggccgcgga
acaacaacaattgcattcattttatgtttcaggttcagggggaggtgtggtcctgcgattccatcgagtg
cacctacaccctgctgaagaccctatgcggcctaagagacctgctaccaatgaattaaaaaaaaatgatt
aataaaaaatcacttacttgaaatcagcaataaggtctctgttgaaattttctcccagcagcacctcact
tccctcttcccaactctggtattctaaaccccgttcagcggcatactttctccatactttaaaggggatg
tcaaattttagctcctctcctgtacccacaatcttcatgtctttcttcccagatgaccaagagagtccgg
ctcagtgactccttcaaccctgtctacccctatgaagatgaaagcacctcccaacaccccttttataacc
cagggtttatttccccaaatggcttcacacaaagcccagacggagttcttactttaaaatgtttaacccc
actaacaaccacaggcggatctctacagctaaaagtgggagggggacttacagtggatgacactgatggt
accttacaagaaaacatacgtgctacagcacccattactaaaaataatcactctgtagaactatccattg
gaaatggattagaaactcaaaacaataaactatgtgccaaattgggaaatgggttaaaatttaacaacgg
tgacatttgtataaaggatagtattaacaccttatggactggaataaaccctccacctaactgtcaaatt
gtggaaaacactaatacaaatgatggcaaacttactttagtattagtaaaaaatggagggcttgttaatg
gctacgtgtctctagttggtgtatcagacactgtgaaccaaatgttcacacaaaagacagcaaacatcca
attaagattatattttgactcttctggaaatctattaactgaggaatcagacttaaaaattccacttaaa
aataaatcttctacagcgaccagtgaaactgtagccagcagcaaagcctttatgccaagtactacagctt
atcccttcaacaccactactagggatagtgaaaactacattcatggaatatgttactacatgactagtta
tgatagaagtctatttcccttgaacatttctataatgctaaacagccgtatgatttcttccaatgttgcc
tatgccatacaatttgaatggaatctaaatgcaagtgaatctccagaaagcaacatagctacgctgacca
catccccctttttcttttcttacattacagaagacgacaactaaaataaagtttaagtgtttttatttaa
aatcacaaaattcgagtagttattttgcctccaccttcccatttgacagaatacacagtcctttctcccc
ggctggccttaaaaagcatcatatcatgggtaacagacatattcttaggtgttatattccacacggtttc
ctgtcgagccaaacgctcatcagtgatattaataaactccccgggcagctcacttaagttcatgtcgctg
tccagctgetgagecacaggctgetgtccaacttgcggttgcttaacgggcggcgaaggagaagtccacg
cctacatgggggtagagtcataatcgtgcatcaggatagggcggtggtgctgcagcagcgcgcgaataaa
ctgctgccgccgccgctccgtcctgcaggaatacaacatggcagtggtctcctcagcgatgattcgcacc
gcccgcagcataaggcgccttgtcctccgggcacagcagcgcaccctgatctcacttaaatcagcacagt
aactgcagcacagcaccacaatattgttcaaaatcccacagtgcaaggcgctgtatccaaagctcatggc
ggggaccacagaacccacgtggccatcataccacaagcgcaggtagattaagtggcgacccctcataaac
acgctggacataaacattacctcttttggcatgttgtaattcaccacctcccggtaccatataaacctct
gattaaacatggcgccatccaccaccatcctaaaccagctggccaaaacctgcccgccggctatacactg
cagggaaccgggactggaacaatgacagtggagagcccaggactcgtaaccatggatcatcatgctcgtc
atgatgtcaatgttggcacaacacaggcacacgtgcatacacttcctcaggattacaagctcctcccgcg
ttagaaccatatcccagggaacaacccattcctgaatcagcgtaaatcccacactgcagggaagacctcg
cacgtaactcacgttgtgcattgtcaaagtgttacattcgggcagcagcggatgatcctccagtatggta
gcgcgggtttctgtctcaaaaggaggtagacgatccctactgtacggagtgcgccgagacaaccgagatc
gtgttggtcgtagtgtcatgccaaatggaacgccggacgtagtcattctcgtattttgtatagcaaaacg
cggccctggcagaacacactcttcttcgccttctatcctgccgcttagcgtgttccgtgtgatagttcaa
gtacagccacactcttaagttggtcaaaagaatgctggcttcagttgtaatcaaaactccatcgcatcta
attgttctgaggaaatcatccacggtagcatatgcaaatcccaaccaagcaatgcaactggattgcgttt
caagcaggagaggagagggaagagacggaagaaccatgttaatttttattccaaacgatctcgcagtact
tcaaattgtagatcgcgcagatggcatctctcgcccccactgtgttggtgaaaaagcacagctaaatcaa
aagaaatgcgattttcaaggtgctcaacggtggcttccaacaaagcctccacgcgcacatccaagaacaa
aagaataccaaaagaaggagcattttctaactcctcaatcatcatattacattcctgcaccattcccaga
taattttcagctttccagccttgaattattcgtgtcagttcttgtggtaaatccaatccacacattacaa
acaggtcccggagggcgccctccaccaccattcttaaacacaccctcataatgacaaaatatcttgctcc
tgtgtcacctgtagcgaattgagaatggcaacatcaattgacatgcccttggctctaagttcttctttaa
gttctagttgtaaaaactctctcatattatcaccaaactgcttagccagaagccccccgggaacaagagc
aggggacgctacagtgcagtacaagcgcagacctccccaattggctccagcaaaaacaagattggaataa
gcatattgggaaccaccagtaatatcatcgaagttgctggaaatataatcaggcagagtttcttgtagaa
attgaataaaagaaaaatttgccaaaaaaacattcaaaacctctgggatgcaaatgcaataggttaccgc
gctgcgctccaacattgttagttttgaattagtctgcaaaaataaaaaaaaaacaagcgtcatatcatag
tagcctgacgaacaggtggataaatcagtctttccatcacaagacaagccacagggtctccagctcgacc
ctcgtaaaacctgtcatcgtgattaaacaacagcaccgaaagttcctcgcggtgaccagcatgaataagt
cttgatgaagcatacaatccagacatgttagcatcagttaaggagaaaaaacagccaacatagcctttgg
gtataattatgcttaatcgtaagtatagcaaagccacccctcgcggatacaaagtaaaaggcacaggaga
ataaaaaatataattatttctctgctgctgtttaggcaacgtcgcccccggtccctctaaatacacatac
aaagcctcatcagccatggcttaccagagaaagtacagcgggcacacaaaccacaagctctaaagtcact
ctccaacctctccacaatatatatacacaagccctaaactgacgtaatgggactaaagtgtaaaaaatcc
cgccaaacccaacacacaccccgaaactgcgtcaccagggaaaagtacagtttcacttccgcaatcccaa
caagcgtcacttcctctttctcacggtacgtcacatcccattaacttacaacgtcattttcccacggccg
cgccgccccttttaaccgttaaccccacagccaatcaccacacggcccacactttttaaaatcacctcat
ttacatattggcaccattccatctataaggtatattattgatgatgtt (SEQ ID NO: 172)
The nucleotide sequence of Helper Vector 3: Ad35E4PS3/WL-ps3:

| | |
|---|---|
| Ad35 1-->154 | Start: 2582 End: 2735 |
| loxP | Start: 2744 End: 2777 |
| Ad35 155-->481 | Start: 2784 End: 3110 |
| loxP | Start: 3111 End: 3144 |
| Ad35 3112-->27435 | Start: 3153 End: 27475 |
| lambda-1 | Start: 27530 End: 29999 (Complementary) |
| BGH polyA | Start: 30313 End: 30527 |
| copGFP | Start: 30552 End: 31217 (Complementary) |
| CMV | Start: 31264 End: 31916 (Complementary) |
| lambda-2 | Start: 31968 End: 33497 |
| Ad35 30544-->31879 | Start: 33558 End: 34893 |
| Ad5E4orf6 | Start: 34889 End: 36003 |
| Ad35 32972-->34794 | Start: 36001 End: 37823 |

taaacttggcgcgccctgagtgatttttctctggtcccgccgcatccataccgccagttgtttaccctca
caacgttccagtaaccgggcatgttcatcatcagtaacccgtatcgtgagcatoctetctegttteateg
gtatcattacccccatgaacagaaatcccccttacacggaggcatcagtgaccaaacaggaaaaaaccgc
ccttaacatggcccgctttatcagaagccagacattaacgcttctggagaaactcaacgagctggacgcg
gatgaacaggcagacatctgtgaatcgcttcacgaccacgctgatgagctttaccgcagctgcctcgcgc
gtttcggtgatgacggtgaaaacctctgacacatgcagctcccggagacggtcacagcttgtctgtaagc
ggatgccgggagcagacaagcccgtcagggcgcgtcagcgggtgttggcgggtgtcggggcgcagccatg
acccagtcacgtagcgatagcggagtgtatactggcttaactatgcggcatcagagcagattgtactgag
agtgcaccatatgcggtgtgaaataccgcacagatgcgtaaggagaaaataccgcatcaggcgctcttcc
gcttcctcgctcactgactcgctgcgctcggtcgttcggctgcggcgagcggtatcagctcactcaaagg
cggtaatacggttatccacagaatcaggggataacgcaggaaagaacatgtgagcaaaaggccagcaaaa
ggccaggaaccgtaaaaaggccgcgttgctggcgtttttccataggctccgcccccctgacgagcatcac
aaaaatcgacgctcaagtcagaggtggcgaaacccgacaggactataaagataccaggcgtttccccctg
gaagctccctcgtgcgctctcctgttccgaccctgcegettaccggatacctgtccgcctttctcccttc
gggaagcgtggcgctttctcatagctcacgctgtaggtatctcagtteggtgtaggtegttegetccaag
ctgggctgtgtgcacgaaccccccgttcagcccgaccgctgcgccttatccggtaactatcgtcttgagt
ccaacccggtaagacacgacttatcgccactggcagcagccactggtaacaggattagcagagcgaggta
tgtaggcggtgctacagagttcttgaagtggtggcctaactacggctacactagaaggacagtatttggt
atctgcgctctgctgaagccagttaccttcggaaaaagagttggtagctcttgatccggcaaacaaacca
ccgctggtagcggtggtttttttgtttgcaagcagcagattacgcgcagaaaaaaaggatctcaagaaga
teetttgatcttttctacggggtctgacgctcagtggaacgaaaactcacgttaagggattttggtcatg
agattatcaaaaaggatcttcacctagatccttttaaattaaaaatgaagttttaaatcaatctaaagta
tatatgagtaaacttggtctgacagttaccaatgcttaatcagtgaggcacctatctcagcgatctgtct
atttegttcatecatagttgcctgactccccgtcgtgtagataactacgatacgggagggcttaccatct
ggccccagtgetgcaatgataccgcgagacccacgctcaccggctccagatttatcagcaataaaccage
cagccggaagggccgagcgcagaagtggtcctgcaactttatccgcctccatccagtctattaattgttg
eegggaagetagagtaagtagttegecagttaatagtttgcgcaacgttgttgccattgetgcaggcatc
gtggtgtcacgctcgtcgtttggtatggcttcattcagctccggttcccaacgatcaaggcgagttacat
gatcccecatgttgtgcaaaaaagcggttagctccttcggtcctccgategttgtcagaagtaagttggc
cgcagtgttatcactcatggttatggcagcactgcataattctcttactgtcatgccatccgtaagatgc
ttttctgtgactggtgagtactcaaccaagtcattctgagaatagtgtatgcggcgaccgagttgctctt
gcccggcgtcaacacgggataataccgcgccacatagcagaactttaaaagtgctcatcattggaaaacg
ttcttcggggcgaaaactctcaaggatcttacegetgttgagatccagttcgatgtaacccactcgtgca
cccaactgatcttcagcatcttttactttcaccagcgtttctgggtgagcaaaaacaggaaggcaaaatg
ccgcaaaaaagggaataagggcgacacggaaatgttgaatactcatactcttcctttttcaatattattg
aagcatttatcagggttattgtctcatgagcggatacatatttgaatgtatttagaaaaataaacaaata
ggggttccgcgcacatttccccgaaaagtgccacctgtctagctacgatatcctgtttaaacatcatcaa
taatataccttatagatggaatggtgccaatatgtaaatgaggtgattttaaaaagtgtgggccgtgtgg
tgattggctgtggggttaacggttaaaaggggcggcgcggccgtgggaaaatgacgttttatgggggtgg
agtttatttaaatataacttcgtatagcatacattatacgaagttatggatccttttgcaagttgtcgcg
ggaaatgttacgcataaaaaggcttcttttctcacggaactacttagttttcccacggtatttaacagga
aatgaggtagttttgaccggatgcaagtgaaaattgctgattttcgcgcgaaaactgaatgaggaagtgt
ttttctgaataatgtggtatttatggcagggtggagtatttgttcagggccaggtagactttgacccatt
acgtggaggtttcgattaccgtgttttttacctgaatttccgcgtaccgtgtcaaagtcttctgttttta
cgtaggtgtcagctgatcgctagggtatttataacttcgtatagcatacattatacgaagttatatttaa
ataggaatgtttatgccttaccagtgtaacatgaatcatgtgaaagtgttgttggaaccagatgcctttt
ccagaatgagcctaacaggaatctttgacatgaacacgcaaatctggaagatcctgaggtatgatgatac
gagatcgagggtgcgcgcatgcgaatgcggaggcaagcatgccaggttccagccggtgtgtgtagatgtg
accgaagatctcagaccggatcatttggttattgcccgcactggagcagagttcggatccagtggagaag
aaactgactaaggtgagtattgggaaaactttggggtgggattttcagatggacagattgagtaaaaatt
tgttttttctgtcttgcagctgacatgagtggaaatgcttcttttaaggggggagtcttcagcccttatc
tgacagggcgtctcccatcctgggcaggagttcgtcagaatgttatgggatctactgtggatggaagacc
cgttcaacccgccaattcttcaacgctgacctatgctactttaagttcttcacctttggacgcagctgca
gccgctgccgccgcctctgtcgccgctaacactgtgcttggaatgggttactatggaagcatcgtggcta
attccacttcctctaataacccttctacactgactcaggacaagttacttgtccttttggcccagctgga
ggctttgacccaacgtctgggtgaactttctcagcaggtggccgagttgcgagtacaaactgagtctgct
gtcggcacggcaaagtctaaataaaaaaaattccagaatcaatgaataaataaacgagcttgttgttgat
ttaaaatcaagtgtttttatttcatttttcgcgcacggtatgccctggaccaccgatctcgatcattgag
aactcggtggattttttccagaatcctatagaggtgggattgaatgtttagatacatgggcattaggccg
tctttggggtggagatagctccattgaagggattcatgctccggggtagtgttgtaaatcacccagtcat
aacaaggtcgcagtgcatggtgttgcacaatatcttttagaagtaggctgattgccacagataagccctt
ggtgtaggtgtttacaaaccggttgagctgggaggggtgcattcgaggtgaaattatgtgcattttggat
tggatttttaagttggcaatattgccgccaagatcccgtcttgggttcatgttatgaaggactaccaaga
cggtgtatccggtacatttaggaaatttatcgtgcagcttggatggaaaagcgtggaaaaatttggagac
acccttgtgtcctccgagattttccatgcactcatccatgataatagcaatggggccgtgggcagcggcg
cgggcaaacacgttccgtgggtctgacacatcatagttatgttcctgagttaaatcatcataagccattt
taatgaatttggggcggagcgtaccagattggggtatgaatgttccttcgggccccggagcatagttccc
ctcacagatttgcatttcccaagctttcagttctgagggtggaatcatgtccacctggggggctatgaag
aacaccgtttcgggggcgggggtgattagttgggatgatagcaagtttctgagcaattgagatttgccac
atccggtggggccataaataattccgattacaggttgcaggtggtagtttagggaacggcaactgccgtc
ttctcgaagcaagggggccacctcgttcatcatttcccttacatgcatattttcccgcaccaaatccatt
aggaggcgctctcctcctagtgatagaagttcttgtagtgaggaaaagtttttcagcggttttagaccgt
cagecatgggcattttggaaagagtttgetgcaaaagttctagtctgttccacagttcagtgatgtgttc
tatggcatctcgatccagcagacctcctcgtttcgcgggtttggacggctcctggagtagggtatgagac
gatgggcgtccagcgctgccagggttcggtccttccagggtctcagtgttcgagtcagggttgtttccgt
cacagtgaaggggtgtgcgcctgcttgggcgcttgccagggtgcgcttcagactcattctgctggtggag
aacttctgtcgcttggcgccctgtatgtcggccaagtagcagtttaccatgagttcgtagttgagcgcct
cggctgcgtggcctttggcgcggagcttacctttggaagttttcttgcataccgggcagtataggcattt
cagcgcatacagcttgggcgcaaggaaaatggattctggggagtatgcatccgcgccgcaggaggcgcaa
acagtttcacattccaccagccaggttaaatccggttcattggggtcaaaaacaagttttccgccatatt
ttttgatgcgtttcttacctttggtctccataagttcgtgtcctcgttgagtgacaaacaggctgtccgt
atctccgtagactgattttacaggcctcttctccagtggagtgcctcggtcttcttcgtacaggaactct
gaccactctgatacaaaggcgcgcgtccaggccagcacaaaggaggctatgtgggaggggtagcgatcgt
tgtcaaccagggggtccaccttttccaaagtatgcaaacacatgtcaccctcttcaacatccaggaatgt
gattggcttgtaggtgtatttcacgtgacctggggtccccgctgggggggtataaaagggggcggttctt
tgctcttcctcactgtcttccggatcgctgtccaggaacgtcagctgttggggtaggtattccctctcga
aggcgggcatgacctctgcactcaggttgtcagtttctaagaacgaggaggatttgatattgacagtgcc
ggttgagatgcctttcatgaggttttcgtccatttggtcagaaaacacaatttttttattgtcaagtttg
gtggcaaatgatccatacagggcgttggataaaagtttggcaatggatcgcatggtttggttcttttcct
tgtccgcgcgctctttggcggcgatgttgagttggacatactcgcgtgccaggcacttccattcggggaa
gatagttgttaattcatctggcacgattctcacttgccaccctcgattatgcaaggtaattaaatccaca
ctggtggccacctcgcctcgaaggggttcattggtccaacagagcctacctcctttcctagaacagaaag
ggggaagtgggtctagcataagttcatcgggagggtctgcatccatggtaaagattcccggaagtaaatc
cttatcaaaatagctgatgggagtggggtcatctaaggccatttgccattctcgagctgccagtgcgcgc
tcatatgggttaaggggactgccccagggcatgggatgggtgagagcagaggcatacatgccacagatgt
catagacgtagatgggatcctcaaagatgcctatgtaggttggatagcatcgcccccctctgatacttgc
tcgcacatagtcatatagttcatgtgatggcgctagcagccccggacccaagttggtgcgattgggtttt
tctgttctgtagacgatctggcgaaagatggcgtgagaattggaagagatggtgggtctttgaaaaatgt
tgaaatgggcatgaggtagacctacagagtctctgacaaagtgggcataagattcttgaagcttggttac
cagttcggcggtgacaagtacgtctagggcgcagtagtcaagtgtttcttgaatgatgtcataacctggt
tggtttttcttttcccacagttcgcggttgagaaggtattcttcgcgatccttccagtactcttctagcg
gaaacccgtctttgtctgcacggtaagatcctagcatgtagaactgattaactgccttgtaagggcagca
gcccttctctacgggtagagagtatgcttgagcagcttttcgtagcgaagcgtgagtaagggcaaaggtg
tctctgaccatgactttgagaaattggtatttgaagtccatgtcgtcacaggctccctgttcccagagtt
ggaagtctacccgtttcttgtaggcggggttgggcaaagcgaaagtaacatcattgaagagaatcttacc
ggctctgggcataaaattgcgagtgatgcggaaaggctgtggtacttccgctcgattgttgatcacctgg
gcagctaggacgatttcgtcgaaaccgttgatgttgtgtcctacgatgtataattctatgaaacgcggcg
tgcctctgacgtgaggtagcttactgagctcatcaaaggttaggtctgtggggtcagataaggcgtagtg
ttcgagagcccattcgtgcaggtgaggatttgcatgtaggaatgatgaccaaagatctaccgccagtgct
gtttgtaactggtcccgatactgacgaaaatgccggccaattgccattttttctggagtgacacagtaga
aggttctggggtcttgttgccatcgatcccacttgagtttaatggctagatcgtgggccatgttgacgag
acgctcttctcctgagagtttcatgaccagcatgaaaggaactagttgtttgccaaaggatcccatccag
gtgtaagtttccacatcgtaggtcaggaagagtctttctgtgcgaggatgagagccgatcgggaagaact
ggatttcctgccaccagttggaggattggctgttgatgtgatggaagtagaagtttctgcggcgcgccga
gcattcgtgtttgtgcttgtacagacggccgcagtagtcgcagcgttgcacgggttgtatctcgtgaatg
agctgtacctggcttcccttgacgagaaatttcagtgggaagccgaggcctggcgattgtatctcgtgct
cttctatattcgctgtatcggcctgttcatcttctgtttcgatggtggtcatgctgacgagcccccgcgg
gaggcaagtccagacctcggcgcgggaggggcggagctgaaggacgagagcgcgcaggctggagctgtcc
agagtcctgagacgctgcggactcaggttagtaggtagggacagaagattaacttgcatgatcttttcca
gggcgtgcgggaggttcagatggtacttgatttccacaggttcgtttgtagagacgtcaatggcttgcag
ggttccgtgtcctttgggcgccactaccgtacctttgttttttcttttgatcggtggtggctctcttgct
tcttgcatgctcagaagcggtgacggggacgcgcgccgggcggcagcggttgttccggacccgggggcat
ggctggtagtggcacgtcggcgccgcgcacgggcaggttctggtattgcgctctgagaagacttgcgtgc
gccaccacgcgtcgattgacgtcttgtatctgacgtctctgggtgaaagctaccggccccgtgagcttga
acctgaaagagagttcaacagaatcaatttcggtatcgttaacggcagcttgtctcagtatttcttgtac
gtcaccagagttgtcctggtaggcgatctccgccatgaactgctcgatttcttcctcctgaagatctccg
cgacccgctctttcgacggtggccgcgaggtcattggagatacggcccatgagttgggagaatgcattca
tgcccgcctcgttccagacgcggctgtaaaccacggccccctcggagtctcttgcgcgcatcaccacctg
agcgaggttaagctccacgtgtctggttaagaccgcatagttgcataggcgctgaaaaaggtagttgagt
gtggtggcaatgtgttcggcgacgaagaaatacatgatccatcgtctcagcggcatttcgctaacatcgc
ccagagcttccaagcgctccatggcctcgtagaagtccacggcaaaattaaaaaactgggagtttcgcgc
ggacacggtcaattcctcctcgagaagacggatgagttcggctatggtggcccgtacttcgcgttcgaag
gctcccgggatctcttcttcctcttctatctcttcttccactaacatctcttcttcgtcttcaggcgggg
gcggagggggcacgcggcgacgtcgacggcgcacgggcaaacggtcgatgaatcgttcaatgacctctcc
gcggcggcggcgcatggtttcagtgacggcgcggccgttctcgcgcggtcgcagagtaaaaacaccgccg
cgcatctccttaaagtggtgactgggaggttctccgtttgggagggagagggcgctgattatacatttta
ttaattggcccgtagggactgcgcgcagagatctgatcgtgtcaagatccacgggatctgaaaacctttc
gacgaaagcgtctaaccagtcacagtcacaaggtaggctgagtacggcttcttgtgggcgggggtggtta
tgtgttcggtctgggtcttctgtttcttcttcatctcgggaaggtgagacgatgctgctggtgatgaaat
taaagtaggcagttctaagacggcggatggtggcgaggagcaccaggtctttgggtccggcttgctggat
acgcaggcgattggccattccccaagcattatcctgacatctagcaagatctttgtagtagtcttgcatg
agccgttctacgggcacttcttcctcacccgttctgccatgcatacgtgtgagtccaaatccgcgcattg
gttgtaccagtgccaagtcagctacgactctttcggcgaggatggcttgctgtacttgggtaagggtggc
ttgaaagtcatcaaaatccacaaagcggtggtaagcccctgtattaatggtgtaagcacagttggccatg
actgaccagttaactgtctggtgaccagggcgcacgagctcggtgtatttaaggcgcgaataggcgcggg
tgtcaaagatgtaatcgttgcaggtgcgcaccagatactggtaccctataagaaaatgcggcggtggttg
gcggtagagaggccatcgttctgtagctggagcgccaggggcgaggtcttccaacataaggcggtgatag
ccgtagatgtacctggacatccaggtgattcctgcggcggtagtagaagcccgaggaaactcgcgtacgc
ggttccaaatgttgcgtagcggcatgaagtagttcattgtaggcacggtttgaccagtgaggcgcgcgca
gtcattgatgctctatagacacggagaaaatgaaagcgttcagcgactcgactccgtagcctggaggaac
gtgaacgggttgggtcgcggtgtaccccggttcgagacttgtactcgagccggccggagccgcggctaac
gtggtattggcactcccgtctcgacccagcctacaaaaatccaggatacggaatcgagtcgttttgctgg
tttccgaatggcagggaagtgagtcctatttttttttttttttgccgctcagaatgcatcccgtgctgcg
acagatgcgcccccaacaacagcccccctcgcagcagcagcagcagcaaccacaaaaggctgtccctgca
actactgcaactgccgccgtgagcggtgcgggacagcccgcctatgatctggacttggaagagggcgaag
gactggcacgtctaggtgcgccttcgcccgagcggcatccgcgagttcaactgaaaaaagattctcgcga
ggcgtatgtgccccaacagaacctatttagagacagaagcggcgaggagccggaggagatgcgagcttcc
cgctttaacgcgggtcgtgagctgcgtcacggtttggaccgaagacgagtgttgcgagacgaggatttcg
aagttgatgaagtgacagggatcagtcctgccagggcacacgtggctgcagccaaccttgtatcggctta
cgagcagacagtaaaggaagagcgtaacttccaaaagtcttttaataatcatgtgcgaaccctgattgcc
cgcgaagaagttacccttggtttgatgcatttgtgggatttgatggaagctatcattcagaaccctacta
gcaaacctctgaccgcccagctgtttctggtggtgcaacacagcagagacaatgaggctttcagagaggc
gctgctgaacatcaccgaacccgaggggagatggttgtatgatcttatcaacattctacagagtatcata
gtgcaggagcggagcctgggcctggccgagaaggtagctgccatcaattactcggttttgagcttgggaa
aatattacgctcgcaaaatctacaagactccatacgttcccatagacaaggaggtgaagatagatgggtt
ctacatgcgcatgacgctcaaggtcttgaccctgagcgatgatcttggggtgtatcgcaatgacagaatg
catcgcgcggttagcgccagcaggaggcgcgagttaagcgacagggaactgatgcacagtttgcaaagag
ctctgactggagctggaaccgagggtgagaattacttcgacatgggagctgacttgcagtggcagcctaa
tcgcagggctctgagcgccgcgacggcaggatgtgagcttccttacatagaagaggcggatgaaggcgag
gaggaagagggcgagtacttggaagactgatggcacaacccgtgttttttgctagatggaacagcaagca
ccggatcccgcaatgcgggcggcgctgcagagccagccgtccggcattaactcctcggacgattggaccc
aggccatgcaacgtatcatggcgttgacgactcgcaaccccgaagcctttagacagcaaccccaggccaa
ccgtctatcggccatcatggaagctgtagtgccttcccgatctaatcccactcatgagaaggtcctggcc
atcgtgaacgcgttggtggagaacaaagctattcgtccagatgaggccggactggtatacaacgctctct
tagaacgcgtggctcgctacaacagtagcaatgtgcaaaccaatttggaccgtatgataacagatgtacg
cgaagccgtgtctcagcgcgaaaggttccagcgtgatgccaacctgggttcgctggtggcgttaaatgct
ttcttgagtactcagectgetaatgtgccgcgtggtcaacaggattatactaactttttaagtgetttga
gactgatggtatcagaagtacctcagagcgaagtgtatcagtccggtcctgattacttctttcagactag
cagacagggcttgcagacggtaaatctgagccaagcttttaaaaaccttaaaggtttgtggggagtgcat
gccccggtaggagaaagagcaaccgtgtctagcttgttaactccgaactcccgcctgttattactgttgg
tagctcctttcaccgacagcggtagcatcgaccgtaattcctatttgggttacctactaaacctgtatcg
cgaagccatagggcaaagtcaggtggacgagcagacctatcaagaaattacccaagtcagtcgcgctttg
ggacaggaagacactggcagtttggaagccactctgaacttcttgcttaccaatcggtctcaaaagatcc
ctcctcaatatgctcttactgcggaggaggagaggatccttagatatgtgcagcagagcgtgggattgtt
tctgatgcaagagggggcaactccgactgcagcactggacatgacagcgcgaaatatggagcccagcatg
tatgccagtaaccgacctttcattaacaaactgctggactacttgcacagagctgccgctatgaactctg
attatttcaccaatgccatcttaaacccgcactggctgcccccacctggtttctacacgggcgaatatga
catgcccgaccctaatgacggatttctgtgggacgacgtggacagcgatgttttttcacctctttctgat
catcgcacgtggaaaaaggaaggcggtgatagaatgcattcttctgcatcgctgtccggggtcatgggtg
ctaccgcggctgagcccgagtctgcaagtccttttcctagtctacccttttctctacacagtgtacgtag
cagcgaagtgggtagaataagtcgcccgagtttaatgggcgaagaggagtacctaaacgattccttgete
agaccggcaagagaaaaaaatttcccaaacaatggaatagaaagtttggtggataaaatgagtagatgga
agacttatgctcaggatcacagagacgagcctgggatcatggggactacaagtagagcgagccgtagacg
ccagcgccatgacagacagaggggtcttgtgtgggacgatgaggattcggccgatgatagcagcgtgttg
gacttgggtgggagaggaaggggcaacccgtttgctcatttgcgccctcgcttgggtggtatgttgtgaa
aaaaaataaaaaagaaaaactcaccaaggccatggcgacgagcgtacgttcgttcttctttattatctgt
gtctagtataatgaggcgagtcgtgctaggcggagcggtggtgtatccggagggtcctcctccttcgtac
gagagcgtgatgcagcagcagcaggcgacggcggtgatgcaatccccactggaggctccctttgtgcctc
cgcgatacctggcacctacggagggcagaaacagcattcgttactcggaactggcacctcagtacgatac
caccaggttgtatctggtggacaacaagtcggcggacattgcttctctgaactatcagaatgaccacagc
aacttcttgaccacggtggtgcagaacaatgactttacccctacggaagccagcacccagaccattaact
ttgatgaacgatcgcggtggggcggtcagctaaagaccatcatgcatactaacatgccaaacgtgaacga
gtatatgtttagtaacaagttcaaagcgcgtgtgatggtgtccagaaaacctcccgacggtgctgcagtt
ggggatacttatgatcacaagcaggatattttggaatatgagtggttcgagtttactttgccagaaggca
acttttcagttactatgactattgatttgatgaacaatgccatcatagataattacttgaaagtgggtag
acagaatggagtgcttgaaagtgacattggtgttaagttcgacaccaggaacttcaagctgggatgggat
cccgaaaccaagttgatcatgcctggagtgtatacgtatgaagccttccatcctgacattgtcttactgc
ctggctgcggagtggattttaccgagagtcgtttgagcaaccttcttggtatcagaaaaaaacagccatt
tcaagagggttttaagattttgtatgaagatttagaaggtggtaatattccggccctcttggatgtagat
gcctatgagaacagtaagaaagaacaaaaagccaaaatagaagctgctacagctgctgcagaagctaagg
caaacatagttgccagcgactctacaagggttgctaacgctggagaggtcagaggagacaattttgcgcc
aacacctgttccgactgcagaatcattattggccgatgtgtctgaaggaacggacgtgaaactcactatt
caacctgtagaaaaagatagtaagaatagaagctataatgtgttggaagacaaaatcaacacagcctatc
gcagttggtatctttcgtacaattatggcgatcccgaaaaaggagtgcgttcctggacattgctcaccac
ctcagatgtcacctgcggagcagagcaggtttactggtcgcttccagacatgatgaaggatcctgtcact
ttccgctccactagacaagtcagtaactaccctgtggtgggtgcagagcttatgcccgtcttctcaaaga
gcttctacaacgaacaagctgtgtactcccagcagctccgccagtccacctcgcttacgcacgtcttcaa
ccgctttcctgagaaccagattttaatccgtccgccggcgcccaccattaccaccgtcagtgaaaacgtt
cctgctctcacagatcacgggaccctgccgttgcgcagcagtatccggggagtccaacgtgtgaccgtta
ctgacgccagacgccgcacctgtccctacgtgtacaaggcactgggcatagtcgcaccgcgcgtcctttc
aagccgcactttctaaaaaaaaaatgtccattcttatctcgcccagtaataacaccggttggggtctgcg
cgctccaagcaagatgtacggaggcgcacgcaaacgttctacccaacatcccgtgcgtgttcgcggacat
tttcgcgctccatggggtgccctcaagggccgcactcgcgttcgaaccaccgtcgatgatgtaategate
aggtggttgccgacgcccgtaattatactcctactgcgcctacatctactgtggatgcagttattgacag
tgtagtggctgacgctcgcaactatgctcgacgtaagagccggcgaaggcgcattgccagacgccaccga
gctaccactgccatgcgagccgcaagagctctgctacgaagagctagacgcgtggggcgaagagccatgc
ttagggcggccagacgtgcagcttcgggcgccagcgccggcaggtcccgcaggcaagcagccgctgtcgc
agcggcgactattgccgacatggcccaatcgcgaagaggcaatgtatactgggtgcgtgacgctgccacc
ggtcaacgtgtacccgtgcgcacccgtccccctcgcacttagaagatactgagcagtctccgatgttgtg
tcccagcggcgaggatgtccaagcgcaaatacaaggaagaaatgctgcaggttatcgcacctgaagtcta
cggccaaccgttgaaggatgaaaaaaaaccccgcaaaatcaagcgggttaaaaaggacaaaaaagaagag
gaagatggcgatgatgggctggcggagtttgtgcgcgagtttgccccacggcgacgcgtgcaatggcgtg
ggcgcaaagttcgacatgtgttgagacctggaacttcggtggtctttacacccggcgagcgttcaagcgc
tacttttaagcgttcctatgatgaggtgtacggggatgatgatattcttgagcaggcggctgaccgatta
ggcgagtttgcttatggcaagcgtagtagaataacttccaaggatgagacagtgtcaatacccttggatc
atggaaatcccacccctagtcttaaaccggtcactttgcagcaagtgttacccgtaactccgcgaacagg
tgttaaacgcgaaggtgaagatttgtatcccactatgcaactgatggtacccaaacgccagaagttggag
gacgttttggagaaagtaaaagtggatccagatattcaacctgaggttaaagtgagacccattaagcagg
tagcgcctggtctgggggtacaaactgtagacattaagattcccactgaaagtatggaagtgcaaactga
acccgcaaagcctactgccacctccactgaagtgcaaacggatccatggatgcccatgcctattacaact
gacgccgccggtcccactcgaagatcccgacgaaagtacggtccagcaagtctgttgatgcccaattatg
ttgtacacccatctattattcctactcctggttaccgaggcactcgctactatcgcagccgaaacagtac
ctcccgccgtcgccgcaagacacctgcaaatcgcagtcgtcgccgtagacgcacaagcaaaccgactccc
ggcgccctggtgcggcaagtgtaccgcaatggtagtgcggaacctttgacactgccgcgtgcgcgttacc
atccgagtatcatcacttaatcaatgttgccgctgcctccttgcagatatggccctcacttgtcgccttc
gcgttcccatcactggttaccgaggaagaaactcgcgccgtagaagagggatgttgggacgcggaatgcg
acgctacaggcgacggcgtgctatccgcaagcaattgcggggtggttttttaccagccttaattccaatt
atcgctgctgcaattggcgcgataccaggcatagcttccgtggcggttcaggcctcgcaacgacattgac
attggaaaaaaaacgtataaataaaaaaaaatacaatggactctgacactcctggtcctgtgactatgtt
ttcttagagatggaagacatcaatttttcatccttggctccgcgacacggcacgaagccgtacatgggca
cctggagcgacatcggcacgagccaactgaacgggggcgccttcaattggagcagtatctggagcgggct
taaaaattttggctcaaccataaaaacatacgggaacaaagcttggaacagcagtacaggacaggcgctt
agaaataaacttaaagaccagaacttccaacaaaaagtagtcgatgggatagcttccggcatcaatggag
tggtagatttggctaaccaggctgtgcagaaaaagataaacagtcgtttggacccgccgccagcaacccc
aggtgaaatgcaagtggaggaagaaattcctccgccagaaaaacgaggcgacaagcgtccgcgtcccgat
ttggaagagacgctggtgacgcgcgtagatgaaccgccttcttatgaggaagcaacgaagcttggaatgc
ccaccactagaccgatagccccaatggccaccggggtgatgaaaccttctcagttgcatcgacccgtcac
cttggatttgccccctccccctgctgctactgctgtacccgcttctaagcctgtcgctgccccgaaacca
gtcgccgtagccaggtcacgtcccgggggcgctcctcgtccaaatgcgcactggcaaaatactctgaaca
gcatcgtgggtctaggcgtgcaaagtgtaaaacgccgtcgctgcttttaattaaatatggagtagcgctt
aacttgcctatctgtgtatatgtgtcattacacgccgtcacagcagcagaggaaaaaaggaagaggtcgt
gcgtcgacgctgagttactttcaagatggccaccccatcgatgctgccccaatgggcatacatgcacatc
gccggacaggatgcttcggagtacctgagtccgggtctggtgcagttcgcccgcgccacagacacctact
tcaatctgggaaataagtttagaaatcccaccgtagcgccgacccacgatgtgaccaccgaccgtagcca
gcggctcatgttgcgcttcgtgcccgttgaccgggaggacaatacatactcttacaaagtgcggtacacc
ctggccgtgggcgacaacagagtgctggatatggccagcacgttctttgacattaggggcgtgttggaca
gaggtcccagtttcaaaccctattctggtacggcttacaactctctggctcctaaaggcgctccaaatgc
atctcaatggattgcaaaaggcgtaccaactgcagcagccgcaggcaatggtgaagaagaacatgaaaca
gaggagaaaactgctacttacacttttgccaatgctcctgtaaaagccgaggctcaaattacaaaagagg
gcttaccaataggtttggagatttcagctgaaaacgaatctaaacccatctatgcagataaactttatca
gccagaacctcaagtgggagatgaaacttggactgacctagacggaaaaaccgaagagtatggaggcagg
gctctaaagcctactactaacatgaaaccctgttacgggtcctatgcgaagcctactaatttaaaaggtg
gtcaggcaaaaccgaaaaactcggaaccgtcgagtgaaaaaattgaatatgatattgacatggaattttt
tgataactcatcgcaaagaacaaacttcagtcctaaaattgtcatgtatgcagaaaatgtaggtttggaa
acgccagacactcatgtagtgtacaaacctggaacagaagacacaagttccgaagctaatttgggacaac
agtctatgcccaacagacccaactacattggcttcagagataactttattggactcatgtactataacag
tactggtaacatgggggtgctggctggtcaagcgtctcagttaaatgcagtggttgacttgcaggacaga
aacacagaactttcttaccaactcttgcttgactctctgggcgacagaaccagatactttagcatgtgga
atcaggctgtggacagttatgatcctgatgtacgtgttattgaaaatcatggtgtggaagatgaacttcc
caactattgttttccactggacggcataggtgttccaacaaccagttacaaatcaatagttccaaatgga
gaagataataataattggaaagaacctgaagtaaatggaacaagtgagatcggacagggtaatttgtttg
ccatggaaattaaccttcaagccaatctatggcgaagtttcctttattccaatgtggctctgtatctccc
agactcgtacaaatacaccccgtccaatgtcactcttccagaaaacaaaaacacctacgactacatgaac
gggcgggtggtgccgccatctctagtagacacctatgtgaacattggtgccaggtggtctctggatgcca
tggacaatgtcaacccattcaaccaccaccgtaacgctggcttgcgttaccgatctatgcttctgggtaa
cggacgttatgtgcctttccacatacaagtgcctcaaaaattcttcgctgttaaaaacctgctgcttctc
ccaggctcctacacttatgagtggaactttaggaaggatgtgaacatggttctacagagttccctcggta
acgacctgcgggtagatggcgccagcatcagtttcacgagcatcaacctctatgctacttttttccccat
ggctcacaacaccgcttccacccttgaagccatgctgcggaatgacaccaatgatcagtcattcaacgac
tacctatctgcagctaacatgctctaccccattcctgccaatgcaaccaatattcccatttccattcctt
ctcgcaactgggcggctttcagaggctggtcatttaccagactgaaaaccaaagaaactccctctttggg
gtctggatttgacccctactttgtctattctggttctattccctacctggatggtaccttctacctgaac
cacacttttaagaaggtttccatcatgtttgactcttcagtgagctggcctggaaatgacaggttactat
ctcctaacgaatttgaaataaagcgcactgtggatggcgaaggctacaacgtagcccaatgcaacatgac
caaagactggttcttggtacagatgctcgccaactacaacatcggctatcagggcttctacattccagaa
ggatacaaagatcgcatgtattcatttttcagaaacttccagcccatgagcaggcaggtggttgatgagg
tcaattacaaagacttcaaggccgtcgccataccctaccaacacaacaactctggctttgtgggttacat
ggctccgaccatgcgccaaggtcaaccctatcccgctaactatccctatccactcattggaacaactgcc
gtaaatagtgttacgcagaaaaagttcttgtgtgacagaaccatgtggcgcataccgttctcgagcaact
tcatgtctatgggggcccttacagacttgggacagaatatgctctatgccaactcagctcatgctctgga
catgacctttgaggtggatcccatggatgagcccaccctgctttatcttctcttcgaagttttcgacgtg
gtcagagtgcatcagccacaccgcggcatcatcgaggcagtctacctgcgtacaccgttctcggccggta
acgctaccacgtaagaagcttcttgcttcttgcaaatagcagctgcaaccatggcctgcggatcccaaaa
cggctccagcgagcaagagctcagagccattgtccaagacctgggttgcggaccctattttttgggaacc
tacgataagcgcttcccggggttcatggcccccgataagctcgcctgtgccattgtaaatacggccggac
gtgagacggggggagagcactggttggctttcggttggaacccacgttctaacacctgctacctttttga
tccttttggattctcggatgatcgtctcaaacagatttaccagtttgaatatgagggtctcctgcgccgc
agcgctcttgctaccaaggaccgctgtattacgctggaaaaatctacccagaccgtgcagggcccccgtt
ctgccgcctgcggacttttctgctgcatgttccttcacgcctttgtgcactggcctgaccgtcccatgga
cggaaaccccaccatgaaattgctaactggagtgccaaacaacatgcttcattctcctaaagtccagccc
accctgtgtgacaatcaaaaagcactctaccattttcttaatacccattcgccttattttcgctctcatc
gtacacacatcgaaagggccactgcgttcgaccgtatggatgttcaataatgactcatgtaaacaacgtg
ttcaataaacatcactttatttttttacatgtatcaaggctctggattacttatttatttacaagtcgaa
tgggttctgacgagaatcagaatgacccgcaggcagtgatacgttgcggaactgatacttgggttgccac
ttgaattcgggaatcaccaacttgggaaccggtatatcgggcaggatgtcactccacagctttctggtca
gctgcaaagctccaagcaggtcaggagccgaaatcttgaaatcacaattaggaccagtgctctgagcgcg
agagttgcggtacaccggattgcagcactgaaacaccatcagcgacggatgtctcacgcttgccagcacg
gtgggatctgcaatcatgcccacatccagatcttcagcattggcaatgctgaacggggtcatcttgcagg
tctgcctacccatggcgggcacccaattaggcttgtggttgcaatcgcagtgcagggggatcagtatcat
cttggcctgatcctgtctgattcctggatacacggctctcatgaaagcatcatattgcttgaaagcctgc
tgggctttactaccctcgggataaaacatcccgcaggacctgctcgaaaactggttagcctgcacagccg
gcatcattcacacagcagcgggcgtcattgttggctatttgcaccacacttctgccccagcggttttggg
tgattttggttcgctcgggattctcctttaaggctcgttgtccgttctcgctggccacatccatctcgat
aatctgctccttctgaatcataatattgccatgcaggcacttcagcttgccctcataatcattgcagcca
tgaggccacaacgcacagcctgtacattcccaattatggtgggcgatctgagaaaaagaatgtatcattc
cctgcagaaatcttcccatcatcgtgctcagtgtcttgtgactagtgaaagttaactggatgcctcggtg
ctcttcgtttacgtactggtgacagatgcgcttgtattgttcgtgttgctcaggcattagtttaaaacag
gttctaagttcgttatccagcctgtacttctccatcagcagacacatcacttccatgcctttctcccaag
cagacaccaggggcaagctaatcggattcttaacagtgcaggcagcagctcctttagccagagggtcatc
tttagcgatcttctcaatgcttcttttgccatccttctcaacgatgcgcacgggcgggtagctgaaaccc
actgctacaagttgcgcctcttctctttcttcttcgctgtcttgactgatgtcttgcatggggatatgtt
tggtcttccttggcttctttttggggggtatcggaggaggaggactgtcgctccgttccggagacaggga
ggattgtgacgtttcgctcaccattaccaactgactgtcggtagaagaacctgaccccacacggcgacag
gtgtttttcttcgggggcagaggtggaggcgattgcgaagggctgcggtccgacctggaaggcggatgac
tggcagaaccccttccgcgttcgggggtgtgctccctgtggcggtcgcttaactgatttccttcgcggct
ggccattgtgttctcctaggcagagaaacaacagacatggaaactcagccattgctgtcaacatcgccac
gagtgccatcacatctcgtcctcagcgacgaggaaaaggagcagagcttaagcattccaccgcccagtcc
tgccaccacctctaccctagaagataaggaggtcgacgcatctcatgacatgcagaataaaaaagcgaaa
gagtctgagacagacatcgagcaagacccgggctatgtgacaccggtggaacacgaggaagagttgaaac
gctttctagagagagaggatgaaaactgcccaaaacagcgagcagataactatcaccaagatgctggaaa
tagggatcagaacaccgactacctcatagggcttgacggggaagacgcgctccttaaacatctagcaaga
cagtcgctcatagtcaaggatgcattattggacagaactgaagtgcccatcagtgtggaagagctcagct
gcgcctacgagcttaaccttttttcacctcgtactccccccaaacgtcagecaaacggcacctgcgagcc
aaatcctcgcttaaacttttatccagcttttgctgtgccagaagtactggctacctatcacatctttttt
aaaaatcaaaaaattccagtctcctgccgcgctaatcgcacccgcgccgatgccctactcaatctgggac
ctggttcacgcttacctgatatagcttccttggaagaggttccaaagatcttcgagggtctgggcaataa
tgagactcgggccgcaaatgetctgcaaaagggagaaaatggcatggatgagcatcacagcgttctggtg
gaattggaaggcgataatgccagactcgcagtactcaagcgaagcgtcgaggtcacacacttcgcatatc
cegetgtcaacctgeeccctaaagtcatgaeggeggtcatggaccagttactcattaagcgcgcaagtcc
cctttcagaagacatgcatgacccagatgcctgtgatgagggtaaaccagtggtcagtgatgagcagcta
acccgatggctgggcaccgactctccccgggatttggaagagcgtcgcaagcttatgatggccgtggtgc
tggttaccgtagaactagagtgtctccgacgtttctttaccgattcagaaaccttgcgcaaactcgaaga
gaatctgcactacacttttagacacggctttgtgcggcaggcatgcaagatatctaacgtggaactcacc
aacctggtttcctacatgggtattctgcatgagaatcgcctaggacaaagcgtgctgcacagcaccctta
agggggaagcccgccgtgattacatccgcgattgtgtctatctctacctgtgccacacgtggcaaaccgg
catgggtgtatggcagcaatgtttagaagaacagaacttgaaagagcttgacaagctcttacagaaatct
cttaaggttctgtggacagggttcgacgagcgcaccgtegettccgacctggcagacctcatcttcccag
agcgtctcagggttactttgcgaaacggattgcctgactttatgagccagagcatgcttaacaattttcg
ctctttcatcctggaacgctccggtatcctgcccgccacctgctgcgcactgccctccgactttgtgcct
ctcacctaccgcgagtgccccccgccgctatggagtcactgctacctgttccgtctggccaactatctct
cctaccactcggatgtgatcgaggatgtgagcggagacggcttgctggagtgccactgccgctgcaatct
gtgcacgccccaccggtccctagcttgcaacccccagttgatgagcgaaacccagataataggcaccttt
gaattgcaaggccccagcagccaaggcgatgggtcttctcctgggcaaagtttaaaactgaccccgggac
tgtggacctccgcctacttgcgcaagtttgctccggaagattaccacccctatgaaatcaagttctatga
ggaccaatcacagcctccaaaggccgaactttcggcttgcgtcatcacccagggggcaattctggcccaa
ttgcaagccatccaaaaatcccgccaagaatttctactgaaaaagggtaagggggtctaccttgaccccc
agaccggcgaggaactcaacacaaggttccctcaggatgtcccaacgacgagaaaacaagaagttgaagg
tgcagccgccgcccccagaagatatggaggaagattgggacagtcaggcagaggaggcggaggaggacag
tctggaggacagtctggaggaagacagtttggaggaggaaaacgaggaggcagaggaggtggaagaagta
accgccgacaaacagttatcctcggctgcggagacaagcaacagcgctaccatctccgctccgagtcgag
gaacccggcggcgtcccagcagtagatgggacgagaccggacgcttcccgaacccaaccagcgcttccaa
gaccggtaagaaggatcggcagggatacaagtcctggcgggggcataagaatgccatcatctcctgcttg
catgagtgcgggggcaacatatccttcacgcggcgctacttgctattccaccatggggtgaactttccgc
gcaatgttttgcattactaccgtcacctccacagcccctactatagccagcaaatcccgacagtctcgac
agataaagacagcggcggcgacctccaacagaaaaccagcagcggcagttagaaaatacacaacaagtgc
agcaacaggaggattaaagattacagccaacgagccagcgcaaacccgagagttaagaaatcggatcttt
ccaaccctgtatgccatcttccagcagagtcggggtcaagagcaggaactgaaaataaaaaaccgatctc
tgcgttcgctcaccagaagttgtttgtatcacaagagcgaagatcaacttcagcgcactctcgaggacgc
cgaggctctcttcaacaagtactgcgcgctgactcttaaagagtaggcagcgaccgcgcttattcaaaaa
aggcgggaattacatcatcctcgacatgagtaaagaaattcccacgccttacatgtggagttatcaaccc
caaatgggattggcagcaggcgcctcccaggactactccacccgcatgaattggctcagcgccgggcctt
ctatgatttctcgagttaatgatatacgcgcctaccgaaaccaaatacttttggaacagtcagctcttac
caccacgccccgccaacaccttaatcccagaaattggcccgccgccctagtgtaccaggaaagtcccgct
cccaccactgtattacttcctcgagacgcccaggccgaagtccaaatgactaatgcaggtgcgcagttag
ctggcggctccaccctatgtcgtcacaggcctcggcataatataaaacgcctgatgatcagaggccgagg
tatccagctcaacgacgagtcggtgagctctccgcttggtctacgaccagacggaatctttcagattgcc
ggctgcgggagatcttccttcacccctcgtcaggctgttctgactttggaaagttcgtcttcgcaacccc
gctcgggcggaatcgggaccgttcaatttgtagaggagtttactccctctgtctacttcaaccccttctc
cggatctcctgggcactacccggacgagttcataccgaacttcgacgcgattagcgagtcagtggacggc
tacgattgatgtctggtgacgcggctgagctatctcggctgcgacatctagaccactgccgccgctttcg
ctgctttgcccgggaacttattgagttcatctacttcgaactccccaaggatcaccctcaaggtccggcc
cacggagtgcggattactatcgaaggcaaaatagactctcgcctgcaacgaattttctcccagcggcccg
tgctgatcgagcgagaccagggaaacaccacggttagtaatcaattacggggtcattagttcatagccca
tatatggagttgcgatcgctgcgggccatgtcatacaccgccttcagagcagccggacctatctgcccgt
tcgtgccgtcgttgttaatcaccacatggttattctgctcaaacgtcccggacgcctgcgaccggctgtc
tgccatgctgcccggtgtaccgacataaccgccggtggcatagccgcgcatcagccggtaaagattcccc
acgccaatccggctggttgcctccttcgtgaagacaaactcaccacggtgaacaatccccgctggctcat
atttgccgccggttcccgtaaatcctccggttgcaaaatggaatttcgccgcagcggcctgaatggctgt
accgcctgacgcggatgcgccgccaccaacagccccgccaatggcgctgccgatactcccgacaatcccc
accattgcctgcttaagcagaatttctgtcatcatggacagcacggaacgggtgaagctgcgccagttct
gctcactgccggtcagcatcgccgccatattctgtgcaataccatcaaaggtctgcgtggctgcactttt
tacctgcgacatactgtccgtggcgctctcttcccactcactccagccggacttcaggcctgccatccag
ttcccgcgaagctggtcttcagccgcccaggtctttttctgctctgacatgacgttattcagcgccagcg
gattatcgccatactgttccttcaggcgctgttccgtggcttcccgttctgcctgccggtcagtcagccc
ccggcttttcgcatcaatggcggcccgttttgcccgttgctgctgtgcgaatttatccgcctgctgcgcc
agcgcgttcaggcgctcctgatacgtaaccttgtcgccaagtgcagccagctggcgtttgtactccagcg
tctcatctttatgcgccagcagggatttctcctgtgcagacagctggcgacgttgcgccgcctcctccag
taccgcgaactgactctccgccttccacaaatcccggcgctgctggctgattttctcatttgctccggca
tgcttctccagcgtccggagttctgcctgaagcgtcagcagggcagcatgagcactgtcttcctgacgat
cgcccgcagacaccttcacgctggactgtttcggctttttcagcgtcgcttcataatcctttttcgccgc
cgccatcagcgtgttgtaatccgcctgcaggattttcccgtctttcagtgccttgttcagttcttcctga
cgggcggtatatttctccagcggcgtctgcagccgttcgtaagccttctgcgcctcttcggtatatttca
gccgtgacgcttcggtatcgctctgctgctgcgcatttttgtcctgttgagtctgctgctcagccttctt
tcgggcggcttcaagcgcaagacgggccttttcacgatcatcccagtaacgcgcccgcgcttcatcgtta
acaaaataatcatccttgcgcagattccagatgtcgtctgctttcttatacgcagcctctgccttaatca
gcatctcctgcgcggtatcaggacgaccaatatccagcaccgcatcccacatggatttgaatgcccgcgc
agtcctgtctgcccaggtctccagcgtgcccatgttctctttcaggcggcgggtctggtcatcaaaccct
ttcgttgcggcctcgttcgccgcctgcaatgccccggcttcatcgccggaacgctgcaactgagcaacat
acgcaatctgctccgccgacacgttatggaactggcgagccatcgccgtcagccccgacgtcgggtctgt
ggtcagcttcccgaaggcttcagcgaccttgtccacctccacgccggatgcagaggagaaacgcgccaca
ctctggctgatggacgcaatctgagcctcaccgcttacccccgccttaaccagtgcgctgagtgactcgc
tggtctggttaaacgtcagccctgccgcctgcccggctctggacaggaccagcatacgatctgccgtcag
tcccgcctgattgccggaaaggaccagcgttttgttgaaatcggacagggttgagttgccctgataccag
gcatacgccagcgcaccggtcgccaccgccagcgaggtggcccccaccatcggcagggtgatcgcaccgg
caagccccctgaacatggggatcatcccgccgaaggagtccttcacctgccccccctgttgcagcaggat
cagccacggactttgcccgcctgcaagctgcgtggccacgtcggtgaactgtgcaggcagcatacgcatg
gcggctttatactgcccgacggaaatccccgctttctgtgcagccagcgcctgtcggctcagcgactgtt
caacgactgccgctgtttttttcgcatcactttccgtaccagaaaaatgacgcctgactctggccatctg
ctcgtcaaatctggccgcatccagactcaaatcaacgacgtcgactaagctctagcatttgtgaaccatc
accctaatcaagttttttggggtcgaggtgccgtaaagcactaaatcggaaccctaaagggagcccccga
tttagagcttgacggggaaagccggcgaacgtggcgagaaaggaagggaagaaagcgaaaggagcgggcg
ctagggcgctggcaagtgtagcggtcacgctgcgcgtaaccaccacacccgccgcgcttaatgcgccgct
acagggcgcgtggggataccccctagagccccagctggttctttccgcctcagaagccatagagcccacc
gcatccccagcatgcctgctattgtcttcccaatcctcccccttgctgtcctgccccaccccacccccca
gaatagaatgacacctactcagacaatgcgatgcaatttcctcattttattaggaaaggacagtgggagt
ggcaccttccagggtcaaggaaggcacgggggaggggcaaacaacagatggctggcaactagaaggcaca
gtcgaggctgatcagcgggtttgctagcttaggcgaaggcgatgggggtcttgaaggcgtgctggtactc
cacgatgcccagctcggtgttgctgtgcagctcctccacgcggcggaaggcgaacatggggcccccgttc
tgcaggatgctggggtggatggcgctcttgaagtgcatgtggctgtccaccacgaagctgtagtagccgc
cgtcgcgcaggctgaaggtgcgggcgaagctgcccaccagcacgttatcgcccatggggtgcaggtgctc
cacggtggcgttgctgcggatgatcttgtcggtgaagatcacgctgtcctcggggaagccggtgcccacc
accttgaagtcgccgatcacgcggccggcctcgtagcggtagctgaagctcacgtgcagcacgccgccgt
cctcgtacttctcgatgcgggtgttggtgtagccgccgttgttgatggcgtgcaggaaggggttctcgta
gccgctggggtaggtgccgaagtggtagaagccgtagcccatcacgtggctcagcaggtaggggctgaag
gtcagggcgcctttggtgctcttcatcttgttggtcatgcggccctgctcgggggtgccctctccgccgc
ccaccagctcgaactccacgccgttcagggtgccggtgatgcggcactcgatcttcatggcgggcatggt
ggctagcctagccagcttgggtctccctatagtgagtcgtattaatttcgataagccagtaagcagtggg
ttctctagttagccagagagctctgcttatatagacctcccaccgtacacgcctaccgcccatttgcgtc
aatggggcggagttgttacgacattttggaaagtcccgttgattttggtgccaaaacaaactcccattga
cgtcaatggggtggagacttggaaatccccgtgagtcaaaccgctatccacgcccattgatgtactgcca
aaaccgcatcaccatggtaatagcgatgactaatacgtagatgtactgccaagtaggaaagtcccataag
gtcatgtactgggcataatgccaggcgggccatttaccgtcattgacgtcaatagggggcgtacttggca
tatgatacacttgatgtactgccaagtgggcagtttaccgtaaatactccacccattgacgtcaatggaa
agtccctattggcgttactatgggaacatacgtcattattgacgtcaatgggcgggggtcgttgggcggt
cagccaggcgggccatttaccgtaagttatgtaacgcggaactccatatatgggctatgaactaatgacc
ccgtaattgattactattaataactacaataatcaatgtcaacgcgtatatctggcccgtacatcgcgaa
gcagcgcaaaacgcctaaccctaagcagattcttcatgcaattaagcttcgcggtgcttcttcagtacgc
tacggcaaatgtcatcgacgtttttatccggaaactgctgtctggctttttttgatttcagaattagcct
gacgggcaatgctgcgaagggcgttttcctgctgaggtgtcattgaacaagtcccatgtcggcaagcata
agcacacagaatatgaagcccgctgccagaaaaatgcattccgtggttgtcatacctggtttctctcatc
tgcttctgctttcgccaccatcatttccagcttttgtgaaagggatgcggctaacgtatgaaattcttcg
tctgtttctactggtattggcacaaacctgattccaatttgagcaaggctatgtgccatctcgatactcg
ttcttaactcaacagaagatgctttgtgcatacagcccctcgtttattatttatctcctcagccagccgc
tgtgctttcagtggatttcggataacagaaaggccgggaaatacccagcctcgctttgtaacggagtaga
cgaaagtgattgcgcctacccggatattatcgtgaggatgcgtcatcgccattgctccccaaatacaaaa
ccaatttcagccagtgcctcgtccattttttcgatgaactccggcacgatctcgtcaaaactcgccatgt
acttttcatcccgctcaatcacgacataatgcaggccttcacgcttcatacgcgggtcatagttggcaaa
gtaccaggcattttttcgcgtcacccacatgctgtactgcacctgggccatgtaagctgactttatggcc
tcgaaaccaccgagccggaacttcatgaaatcccgggaggtaaacgggcatttcagttcaaggccgttgc
cgtcactgcataaaccatcgggagagcaggcggtacgcatactttcgtcgcgatagatgatcggggattc
agtaacattcacgccggaagtgaattcaaacagggttctggcgtcgttctcgtactgttttccccaggcc
agtgctttagcgttaacttccggagccacaccggtgcaaacctcagcaagcagggtgtggaagtaggaca
ttttcatgtcaggccacttctttccggagcggggttttgctatcacgttgtgaacttctgaagcggtgat
gacgccgagccgtaatttgtgccacgcatcatccccctgttcgacagctctcacatcgatcccggtacgc
tgcaggataatgtccggtgtcatgctgccaccttctgctctgcggctttctgtttcaggaatccaagagc
ttttactgcttcggcctgtgtcagttctgacgatgcacgaatgtcgcggcgaaatatctgggaacagagc
ggcaataagtcgtcatcccatgttttatccagggcgatcagcagagtgttaatctcctgcatggtttcat
cgttaaccggagtgatgtcgcgttccggctgacgttctgcagtgtatgcagtattttcgacaatgcgctc
ggcttcatccttgtcatagataccagcaaatccgaagcggccgcggaacaacaacaattgcattcatttt
atgtttcaggttcagggggaggtgtggtcctgcgattccatcgagtgcacctacaccctgctgaagaccc
tatgcggcctaagagacctgctaccaatgaattaaaaaaaaatgattaataaaaaatcacttacttgaaa
tcagcaataaggtctctgttgaaattttctcccagcagcacctcacttccctcttcccaactctggtatt
ctaaaccccgttcagcggcatactttctccatactttaaaggggatgtcaaattttagctcctctcctgt
acccacaatcttcatgtctttcttcccagatgaccaagagagtccggctcagtgactccttcaaccctgt
ctacccctatgaagatgaaagcacctcccaacaccccttttataacccagggtttatttccccaaatggc
ttcacacaaagcccagacggagttcttactttaaaatgtttaaccccactaacaaccacaggcggatctc
tacagctaaaagtgggagggggacttacagtggatgacactgatggtaccttacaagaaaacatacgtgc
tacagcacccattactaaaaataatcactctgtagaactatccattggaaatggattagaaactcaaaac
aataaactatgtgccaaattgggaaatgggttaaaatttaacaacggtgacatttgtataaaggatagta
ttaacaccttatggactggaataaaccctccacctaactgtcaaattgtggaaaacactaatacaaatga
tggcaaacttactttagtattagtaaaaaatggagggcttgttaatggctacgtgtctctagttggtgta
tcagacactgtgaaccaaatgttcacacaaaagacagcaaacatccaattaagattatattttgactctt
ctggaaatctattaactgaggaatcagacttaaaaattccacttaaaaataaatcttctacagcgaccag
tgaaactgtagccagcagcaaagcctttatgccaagtactacagcttatcccttcaacaccactactagg
gatagtgaaaactacattcatggaatatgttactacatgactagttatgatagaagtctatttcccttga
acatttctataatgctaaacagccgtatgatttcttccaatgttgcctatgccatacaatttgaatggaa
tctaaatgcaagtgaatctccagaaagcaacatagctacgctgaccacatccccctttttcttttcttac
attacagaagacgacaactaaaataaagtttaagtgtttttatttaaaatcacaaaattcgagtagttat
tttgcctccaccttcccatttgacagaatacacagtcctttctccccggctggccttaaaaagcatcata
tcatgggtaacagacatattcttaggtgttatattccacacggtttcctgtcgagccaaacgctcatcag
tgatattaataaactccccgggcagctcacttaagttcatgtcgctgtccagctgctgagccacaggctg
ctgtccaacttgcggttgcttaacgggcggcgaaggagaagtccacgcctacatgggggtagagtcataa
tcgtgcatcaggatagggcggtggtgctgcagcagcgcgcgaataaactgctgccgccgccgctccgtcc
tgcaggaatacaacatggcagtggtctcctcagcgatgattcgcaccgcccgcagcataaggcgccttgt
cctccgggcacagcagcgcaccctgatctcacttaaatcagcacagtaactgcagcacagcaccacaata
ttgttcaaaatcccacagtgcaaggcgctgtatccaaagctcatggcggggaccacagaacccacgtggc
catcataccacaagcgcaggtagattaagtggcgacccctcataaacacgctggacataaacattacctc
ttttggcatgttgtaattcaccacctcccggtaccatataaacctctgattaaacatggcgccatccacc
accatcctaaaccagctggccaaaacctgcccgccggctatacactgcagggaaccgggactggaacaat
gacagtggagagcccaggactcgtaaccatggatcatcatgctcgtcatgatgtcaatgttggcacaaca
caggcacacgtgcatacacttcctcaggattacaagctcctcccgcgttagaaccatatcccagggaaca
acccattcctgaatcagcgtaaatcccacactgcagggaagacctcgcacgtaactcacgttgtgcattg
tcaaagtgttacattcgggcagcagcggatgatcctccagtatggtagcgcgggtttctgtctcaaaagg
aggtagacgatccctactgtacggagtgcgccgagacaaccgagatcgtgttggtcgtagtgtcatgcca
aatggaacgccggacgtagtcattctcgtattttgtatagcaaaacgcggccctggcagaacacactctt
cttcgccttctatcctgccgcttagcgtgttccgtgtgatagttcaagtacagccacactcttaagttgg
tcaaaagaatgctggcttcagttgtaatcaaaactccatcgcatctaattgttctgaggaaatcatccac
ggtagcatatgcaaatcccaaccaagcaatgcaactggattgcgtttcaagcaggagaggagagggaaga
gacggaagaaccatgttaatttttattccaaacgatctcgcagtacttcaaattgtagatcgcgcagatg
gcatctctcgcccccactgtgttggtgaaaaagcacagctaaatcaaaagaaatgcgattttcaaggtgc
tcaacggtggcttccaacaaagcctccacgcgcacatccaagaacaaaagaataccaaaagaaggagcat
tttctaactcctcaatcatcatattacattcctgcaccattcccagataattttcagctttccagccttg
aattattcgtgtcagttcttgtggtaaatccaatccacacattacaaacaggtcccggagggcgccctcc
accaccattcttaaacacaccctcataatgacaaaatatcttgctcctgtgtcacctgtagcgaattgag
aatggcaacatcaattgacatgcccttggctctaagttcttctttaagttctagttgtaaaaactctctc
atattatcaccaaactgcttagccagaagccccccgggaacaagagcaggggacgctacagtgcagtaca
agcgcagacctccccaattggctccagcaaaaacaagattggaataagcatattgggaaccaccagtaat
atcatcgaagttgctggaaatataatcaggcagagtttcttgtagaaattgaataaaagaaaaatttgcc
aaaaaaacattcaaaacctctgggatgcaaatgcaataggttaccgcgctgcgctccaacattgttagtt
ttgaattagtctgcaaaaataaaaaaaaaacaagcgtcatatcatagtagcctgacgaacaggtggataa
atcagtctttccatcacaagacaagccacagggtctccagctcgaccctcgtaaaacctgtcatcgtgat
taaacaacagcaccgaaagttcctcgcggtgaccagcatgaataagtcttgatgaagcatacaatccaga
catgttagcatcagttaaggagaaaaaacagccaacatagcctttgggtataattatgcttaatcgtaag
tatagcaaagccacccctcgcggatacaaagtaaaaggcacaggagaataaaaaatataattatttctct
getgetgtttaggcaacgtcgcccccggtccctctaaatacacatacaaagcctcatcagecatggetta
ccagagaaagtacagcgggcacacaaaccacaagctctaaagtcactctccaacctctccacaatatata
tacacaagccctaaactgacgtaatgggactaaagtgtaaaaaatcccgccaaacccaacacacaccccg
aaactgcgtcaccagggaaaagtacagtttcacttccgcaatcccaacaagcgtcacttcctctttctca
cggtacgtcacatcccattaacttacaacgtcattttcccacggccgcgcegeeccttttaaccgttaac
cccacagccaatcaccacacggcccacactttttaaaatcacctcatttacatattggcaccattccatc
tataaggtatattattgatgatgtt (SEQ ID NO: 173)
The nucleotide sequence of pAd35GFP-5E4-CCDb:
CATCATCAATAATATACCTTATAGATGGAATGGTGCCAATATGTAAATGAGGTGATTTTAAAAAGTGTGG
GCCGTGTGGTGATTGGCTGTGGGGTTAACGGTTAAAAGGGGCGGCGCGGCCGTGGGAAAATGACGTTTTA
TGGGGGTGGAGTTTTTTTGCAAGTTGTCGCGGGAAATGTTACGCATAAAAAGGCTTCTTTTCTCACGGAA
CTACTTAGTTTTCCCACGGTATTTAACAGGAAATGAGGTAGTTTTGACCGGATGCAAGTGAAAATTGCTG
ATTTTCGCGCGAAAACTGAATGAGGAAGTGTTTTTCTGAATAATGTGGTATTTATGGCAGGGTGGAGTAT
TTGTTCAGGGCCAGGTAGACTTTGACCCATTACGTGGAGGTTTCGATTACCGTGTTTTTTACCTGAATTT
CCGCGTACCGTGTCAAAGTCTTCTGTTTTTACGTAGGTGTCAGCTGATCGCTAGGGTATTTGTTCAAAAA
AAAGCCCGCTCATTAGGCGGGCTGGGTTATATTCCCCAGAACATCAGGTTAATGGCGTTTTTGATGTCAT
TTTCGCGGTGGCTGAGATCAGCCACTTCTTCCCCGATAACGGAGACCGGCACACTGGCCATATCGGTGGT
CATCATGCGCCAGCTTTCATCCCCGATATGCACCACCGGGTAAAGTTCACGGGAGACTTTATCTGACAGC
AGACGTGCACTGGCCAGGGGGATCACCATCCGTCGCCCGGGCGTGTCAATAATATCACTCTGTACATCCA
CAAACAGACGATAACGGCTCTCTCTTTTATAGGTGTAAACCTTAAACTGCATAATCTGACCTCCTGGTTA
TGTGTGGGAGGGCTAACCATGGATCCATGGTTACCAATGCTTAATCAGTGAGGCACCTATCTCAGCGATC
TGTCTATTTCGTTCATCCATAGTTGCCTGACTCCCCGTCGTGTAGATAACTACGATACGGGAGGGCTTAC
CATCTGGCCCCAGTGCTGCAATGATACCGCGAGACCCACGCTCACCGGCTCCAGATTTATCAGCAATAAA
CCAGCCAGCCGGAAGGGCCGAGCGCAGAAGTGGTCCTGCAACTTTATCCGCCTCCATCCAGTCTATTAAT
TGTTGCCGGGAAGCTAGAGTAAGTAGTTCGCCAGTTAATAGTTTGCGCAACGTTGTTGCCATTGCTGCAG
GCATCGTGGTGTCACGCTCGTCGTTTGGTATGGCTTCATTCAGCTCCGGTTCCCAACGATCAAGGCGAGT
TACATGATCCCCCATGTTGTGCAAAAAAGCGGTTAGCTCCTTCGGTCCTCCGATCGTTGTCAGAAGTAAG
TTGGCCGCAGTGTTATCACTCATGGTTATGGCAGCACTGCATAATTCTCTTACTGTCATGCCATCCGTAA
GATGCTTTTCTGTGACTGGTGAGTACTCAACCAAGTCATTCTGAGAATAGTGTATGCGGCGACCGAGTTG
CTCTTGCCCGGCGTCAACACGGGATAATACCGCGCCACATAGCAGAACTTTAAAAGTGCTCATCATTGGA
AAACGTTCTTCGGGGCGAAAACTCTCAAGGATCTTACCGCTGTTGAGATCCAGTTCGATGTAACCCACTC
GTGCACCCAACTGATCTTCAGCATCTTTTACTTTCACCAGCGTTTCTGGGTGAGCAAAAACAGGAAGGCA
AAATGCCGCAAAAAAGGGAATAAGGGCGACACGGAAATGTTGAATACTCATACTCTTCCTTTTTCAATAT
TATTGAAGCATTTATCAGGGTTATTGTCTCATGAGCGGATACATATTTGAATGTATTTAGAAAAATAAAC
AAAGCTACCTTAAGAGAGTGTCAGGAATGTTTATGCCTTACCAGTGTAACATGAATCATGTGAAAGTGTT
GTTGGAACCAGATGCCTTTTCCAGAATGAGCCTAACAGGAATCTTTGACATGAACACGCAAATCTGGAAG
ATCCTGAGGTATGATGATACGAGATCGAGGGTGCGCGCATGCGAATGCGGAGGCAAGCATGCCAGGTTCC
AGCCGGTGTGTGTAGATGTGACCGAAGATCTCAGACCGGATCATTTGGTTATTGCCCGCACTGGAGCAGA
GTTCGGATCCAGTGGAGAAGAAACTGACTAAGGTGAGTATTGGGAAAACTTTGGGGTGGGATTTTCAGAT
GGACAGATTGAGTAAAAATTTGTTTTTTCTGTCTTGCAGCTGACATGAGTGGAAATGCTTCTTTTAAGGG
GGGAGTCTTCAGCCCTTATCTGACAGGGCGTCTCCCATCCTGGGCAGGAGTTCGTCAGAATGTTATGGGA
TCTACTGTGGATGGAAGACCCGTTCAACCCGCCAATTCTTCAACGCTGACCTATGCTACTTTAAGTTCTT
CACCTTTGGACGCAGCTGCAGCCGCTGCCGCCGCCTCTGTCGCCGCTAACACTGTGCTTGGAATGGGTTA
CTATGGAAGCATCGTGGCTAATTCCACTTCCTCTAATAACCCTTCTACACTGACTCAGGACAAGTTACTT
GTCCTTTTGGCCCAGCTGGAGGCTTTGACCCAACGTCTGGGTGAACTTTCTCAGCAGGTGGCCGAGTTGC
GAGTACAAACTGAGTCTGCTGTCGGCACGGCAAAGTCTAAATAAAAAAAATTCCAGAATCAATGAATAAA
TAAACGAGCTTGTTGTTGATTTAAAATCAAGTGTTTTTATTTCATTTTTCGCGCACGGTATGCCCTGGAC
CACCGATCTCGATCATTGAGAACTCGGTGGATTTTTTCCAGAATCCTATAGAGGTGGGATTGAATGTTTA
GATACATGGGCATTAGGCCGTCTTTGGGGTGGAGATAGCTCCATTGAAGGGATTCATGCTCCGGGGTAGT
GTTGTAAATCACCCAGTCATAACAAGGTCGCAGTGCATGGTGTTGCACAATATCTTTTAGAAGTAGGCTG
ATTGCCACAGATAAGCCCTTGGTGTAGGTGTTTACAAACCGGTTGAGCTGGGAGGGGTGCATTCGAGGTG
AAATTATGTGCATTTTGGATTGGATTTTTAAGTTGGCAATATTGCCGCCAAGATCCCGTCTTGGGTTCAT
GTTATGAAGGACTACCAAGACGGTGTATCCGGTACATTTAGGAAATTTATCGTGCAGCTTGGATGGAAAA
GCGTGGAAAAATTTGGAGACACCCTTGTGTCCTCCGAGATTTTCCATGCACTCATCCATGATAATAGCAA
TGGGGCCGTGGGCAGCGGCGCGGGCAAACACGTTCCGTGGGTCTGACACATCATAGTTATGTTCCTGAGT
TAAATCATCATAAGCCATTTTAATGAATTTGGGGCGGAGCGTACCAGATTGGGGTATGAATGTTCCTTCG
GGCCCCGGAGCATAGTTCCCCTCACAGATTTGCATTTCCCAAGCTTTCAGTTCTGAGGGTGGAATCATGT
CCACCTGGGGGGCTATGAAGAACACCGTTTCGGGGGCGGGGGTGATTAGTTGGGATGATAGCAAGTTTCT
GAGCAATTGAGATTTGCCACATCCGGTGGGGCCATAAATAATTCCGATTACAGGTTGCAGGTGGTAGTTT
AGGGAACGGCAACTGCCGTCTTCTCGAAGCAAGGGGGCCACCTCGTTCATCATTTCCCTTACATGCATAT
TTTCCCGCACCAAATCCATTAGGAGGCGCTCTCCTCCTAGTGATAGAAGTTCTTGTAGTGAGGAAAAGTT
TTTCAGCGGTTTTAGACCGTCAGCCATGGGCATTTTGGAAAGAGTTTGCTGCAAAAGTTCTAGTCTGTTC
CACAGTTCAGTGATGTGTTCTATGGCATCTCGATCCAGCAGACCTCCTCGTTTCGCGGGTTTGGACGGCT
CCTGGAGTAGGGTATGAGACGATGGGCGTCCAGCGCTGCCAGGGTTCGGTCCTTCCAGGGTCTCAGTGTT
CGAGTCAGGGTTGTTTCCGTCACAGTGAAGGGGTGTGCGCCTGCTTGGGCGCTTGCCAGGGTGCGCTTCA
GACTCATTCTGCTGGTGGAGAACTTCTGTCGCTTGGCGCCCTGTATGTCGGCCAAGTAGCAGTTTACCAT
GAGTTCGTAGTTGAGCGCCTCGGCTGCGTGGCCTTTGGCGCGGAGCTTACCTTTGGAAGTTTTCTTGCAT
ACCGGGCAGTATAGGCATTTCAGCGCATACAGCTTGGGCGCAAGGAAAATGGATTCTGGGGAGTATGCAT
CCGCGCCGCAGGAGGCGCAAACAGTTTCACATTCCACCAGCCAGGTTAAATCCGGTTCATTGGGGTCAAA
AACAAGTTTTCCGCCATATTTTTTGATGCGTTTCTTACCTTTGGTCTCCATAAGTTCGTGTCCTCGTTGA
GTGACAAACAGGCTGTCCGTATCTCCGTAGACTGATTTTACAGGCCTCTTCTCCAGTGGAGTGCCTCGGT
CTTCTTCGTACAGGAACTCTGACCACTCTGATACAAAGGCGCGCGTCCAGGCCAGCACAAAGGAGGCTAT
GTGGGAGGGGTAGCGATCGTTGTCAACCAGGGGGTCCACCTTTTCCAAAGTATGCAAACACATGTCACCC
TCTTCAACATCCAGGAATGTGATTGGCTTGTAGGTGTATTTCACGTGACCTGGGGTCCCCGCTGGGGGGG
TATAAAAGGGGGCGGTTCTTTGCTCTTCCTCACTGTCTTCCGGATCGCTGTCCAGGAACGTCAGCTGTTG
GGGTAGGTATTCCCTCTCGAAGGCGGGCATGACCTCTGCACTCAGGTTGTCAGTTTCTAAGAACGAGGAG
GATTTGATATTGACAGTGCCGGTTGAGATGCCTTTCATGAGGTTTTCGTCCATTTGGTCAGAAAACACAA
TTTTTTTATTGTCAAGTTTGGTGGCAAATGATCCATACAGGGCGTTGGATAAAAGTTTGGCAATGGATCG
CATGGTTTGGTTCTTTTCCTTGTCCGCGCGCTCTTTGGCGGCGATGTTGAGTTGGACATACTCGCGTGCC
AGGCACTTCCATTCGGGGAAGATAGTTGTTAATTCATCTGGCACGATTCTCACTTGCCACCCTCGATTAT
GCAAGGTAATTAAATCCACACTGGTGGCCACCTCGCCTCGAAGGGGTTCATTGGTCCAACAGAGCCTACC
TCCTTTCCTAGAACAGAAAGGGGGAAGTGGGTCTAGCATAAGTTCATCGGGAGGGTCTGCATCCATGGTA
AAGATTCCCGGAAGTAAATCCTTATCAAAATAGCTGATGGGAGTGGGGTCATCTAAGGCCATTTGCCATT
CTCGAGCTGCCAGTGCGCGCTCATATGGGTTAAGGGGACTGCCCCAGGGCATGGGATGGGTGAGAGCAGA
GGCATACATGCCACAGATGTCATAGACGTAGATGGGATCCTCAAAGATGCCTATGTAGGTTGGATAGCAT
CGCCCCCCTCTGATACTTGCTCGCACATAGTCATATAGTTCATGTGATGGCGCTAGCAGCCCCGGACCCA
AGTTGGTGCGATTGGGTTTTTCTGTTCTGTAGACGATCTGGCGAAAGATGGCGTGAGAATTGGAAGAGAT
GGTGGGTCTTTGAAAAATGTTGAAATGGGCATGAGGTAGACCTACAGAGTCTCTGACAAAGTGGGCATAA
GATTCTTGAAGCTTGGTTACCAGTTCGGCGGTGACAAGTACGTCTAGGGCGCAGTAGTCAAGTGTTTCTT
GAATGATGTCATAACCTGGTTGGTTTTTCTTTTCCCACAGTTCGCGGTTGAGAAGGTATTCTTCGCGATC
CTTCCAGTACTCTTCTAGCGGAAACCCGTCTTTGTCTGCACGGTAAGATCCTAGCATGTAGAACTGATTA
ACTGCCTTGTAAGGGCAGCAGCCCTTCTCTACGGGTAGAGAGTATGCTTGAGCAGCTTTTCGTAGCGAAG
CGTGAGTAAGGGCAAAGGTGTCTCTGACCATGACTTTGAGAAATTGGTATTTGAAGTCCATGTCGTCACA
GGCTCCCTGTTCCCAGAGTTGGAAGTCTACCCGTTTCTTGTAGGCGGGGTTGGGCAAAGCGAAAGTAACA
TCATTGAAGAGAATCTTACCGGCTCTGGGCATAAAATTGCGAGTGATGCGGAAAGGCTGTGGTACTTCCG
CTCGATTGTTGATCACCTGGGCAGCTAGGACGATTTCGTCGAAACCGTTGATGTTGTGTCCTACGATGTA
TAATTCTATGAAACGCGGCGTGCCTCTGACGTGAGGTAGCTTACTGAGCTCATCAAAGGTTAGGTCTGTG
GGGTCAGATAAGGCGTAGTGTTCGAGAGCCCATTCGTGCAGGTGAGGATTTGCATGTAGGAATGATGACC
AAAGATCTACCGCCAGTGCTGTTTGTAACTGGTCCCGATACTGACGAAAATGCCGGCCAATTGCCATTTT
TTCTGGAGTGACACAGTAGAAGGTTCTGGGGTCTTGTTGCCATCGATCCCACTTGAGTTTAATGGCTAGA
TCGTGGGCCATGTTGACGAGACGCTCTTCTCCTGAGAGTTTCATGACCAGCATGAAAGGAACTAGTTGTT
TGCCAAAGGATCCCATCCAGGTGTAAGTTTCCACATCGTAGGTCAGGAAGAGTCTTTCTGTGCGAGGATG
AGAGCCGATCGGGAAGAACTGGATTTCCTGCCACCAGTTGGAGGATTGGCTGTTGATGTGATGGAAGTAG
AAGTTTCTGCGGCGCGCCGAGCATTCGTGTTTGTGCTTGTACAGACGGCCGCAGTAGTCGCAGCGTTGCA
CGGGTTGTATCTCGTGAATGAGCTGTACCTGGCTTCCCTTGACGAGAAATTTCAGTGGGAAGCCGAGGCC
TGGCGATTGTATCTCGTGCTCTTCTATATTCGCTGTATCGGCCTGTTCATCTTCTGTTTCGATGGTGGTC
ATGCTGACGAGCCCCCGCGGGAGGCAAGTCCAGACCTCGGCGCGGGAGGGGCGGAGCTGAAGGACGAGAG
CGCGCAGGCTGGAGCTGTCCAGAGTCCTGAGACGCTGCGGACTCAGGTTAGTAGGTAGGGACAGAAGATT
AACTTGCATGATCTTTTCCAGGGCGTGCGGGAGGTTCAGATGGTACTTGATTTCCACAGGTTCGTTTGTA
GAGACGTCAATGGCTTGCAGGGTTCCGTGTCCTTTGGGCGCCACTACCGTACCTTTGTTTTTTCTTTTGA
TCGGTGGTGGCTCTCTTGCTTCTTGCATGCTCAGAAGCGGTGACGGGGACGCGCGCCGGGCGGCAGCGGT
TGTTCCGGACCCGGGGGCATGGCTGGTAGTGGCACGTCGGCGCCGCGCACGGGCAGGTTCTGGTATTGCG
CTCTGAGAAGACTTGCGTGCGCCACCACGCGTCGATTGACGTCTTGTATCTGACGTCTCTGGGTGAAAGC
TACCGGCCCCGTGAGCTTGAACCTGAAAGAGAGTTCAACAGAATCAATTTCGGTATCGTTAACGGCAGCT
TGTCTCAGTATTTCTTGTACGTCACCAGAGTTGTCCTGGTAGGCGATCTCCGCCATGAACTGCTCGATTT
CTTCCTCCTGAAGATCTCCGCGACCCGCTCTTTCGACGGTGGCCGCGAGGTCATTGGAGATACGGCCCAT
GAGTTGGGAGAATGCATTCATGCCCGCCTCGTTCCAGACGCGGCTGTAAACCACGGCCCCCTCGGAGTCT
CTTGCGCGCATCACCACCTGAGCGAGGTTAAGCTCCACGTGTCTGGTGAAGACCGCATAGTTGCATAGGC
GCTGAAAAAGGTAGTTGAGTGTGGTGGCAATGTGTTCGGCGACGAAGAAATACATGATCCATCGTCTCAG
CGGCATTTCGCTAACATCGCCCAGAGCTTCCAAGCGCTCCATGGCCTCGTAGAAGTCCACGGCAAAATTA
AAAAACTGGGAGTTTCGCGCGGACACGGTCAATTCCTCCTCGAGAAGACGGATGAGTTCGGCTATGGTGG
CCCGTACTTCGCGTTCGAAGGCTCCCGGGATCTCTTCTTCCTCTTCTATCTCTTCTTCCACTAACATCTC
TTCTTCGTCTTCAGGCGGGGGCGGAGGGGGCACGCGGCGACGTCGACGGCGCACGGGCAAACGGTCGATG
AATCGTTCAATGACCTCTCCGCGGCGGCGGCGCATGGTTTCAGTGACGGCGCGGCCGTTCTCGCGCGGTC
GCAGAGTAAAAACACCGCCGCGCATCTCCTTAAAGTGGTGACTGGGAGGTTCTCCGTTTGGGAGGGAGAG
GGCGCTGATTATACATTTTATTAATTGGCCCGTAGGGACTGCGCGCAGAGATCTGATCGTGTCAAGATCC
ACGGGATCTGAAAACCTTTCGACGAAAGCGTCTAACCAGTCACAGTCACAAGGTAGGCTGAGTACGGCTT
CTTGTGGGCGGGGGTGGTTATGTGTTCGGTCTGGGTCTTCTGTTTCTTCTTCATCTCGGGAAGGTGAGAC
GATGCTGCTGGTGATGAAATTAAAGTAGGCAGTTCTAAGACGGCGGATGGTGGCGAGGAGCACCAGGTCT
TTGGGTCCGGCTTGCTGGATACGCAGGCGATTGGCCATTCCCCAAGCATTATCCTGACATCTAGCAAGAT
CTTTGTAGTAGTCTTGCATGAGCCGTTCTACGGGCACTTCTTCCTCACCCGTTCTGCCATGCATACGTGT
GAGTCCAAATCCGCGCATTGGTTGTACCAGTGCCAAGTCAGCTACGACTCTTTCGGCGAGGATGGCTTGC
TGTACTTGGGTAAGGGTGGCTTGAAAGTCATCAAAATCCACAAAGCGGTGGTAAGCCCCTGTATTAATGG
TGTAAGCACAGTTGGCCATGACTGACCAGTTAACTGTCTGGTGACCAGGGCGCACGAGCTCGGTGTATTT
AAGGCGCGAATAGGCGCGGGTGTCAAAGATGTAATCGTTGCAGGTGCGCACCAGATACTGGTACCCTATA
AGAAAATGCGGCGGTGGTTGGCGGTAGAGAGGCCATCGTTCTGTAGCTGGAGCGCCAGGGGCGAGGTCTT
CCAACATAAGGCGGTGATAGCCGTAGATGTACCTGGACATCCAGGTGATTCCTGCGGCGGTAGTAGAAGC
CCGAGGAAACTCGCGTACGCGGTTCCAAATGTTGCGTAGCGGCATGAAGTAGTTCATTGTAGGCACGGTT
TGACCAGTGAGGCGCGCGCAGTCATTGATGCTCTATAGACACGGAGAAAATGAAAGCGTTCAGCGACTCG
ACTCCGTAGCCTGGAGGAACGTGAACGGGTTGGGTCGCGGTGTACCCCGGTTCGAGACTTGTACTCGAGC
CGGCCGGAGCCGCGGCTAACGTGGTATTGGCACTCCCGTCTCGACCCAGCCTACAAAAATCCAGGATACG
GAATCGAGTCGTTTTGCTGGTTTCCGAATGGCAGGGAAGTGAGTCCTATTTTTTTTTTTTTTGCCGCTCA
GATGCATCCCGTGCTGCGACAGATGCGCCCCCAACAACAGCCCCCCTCGCAGCAGCAGCAGCAGCAACCA
CAAAAGGCTGTCCCTGCAACTACTGCAACTGCCGCCGTGAGCGGTGCGGGACAGCCCGCCTATGATCTGG
ACTTGGAAGAGGGCGAAGGACTGGCACGTCTAGGTGCGCCTTCGCCCGAGCGGCATCCGCGAGTTCAACT
GAAAAAAGATTCTCGCGAGGCGTATGTGCCCCAACAGAACCTATTTAGAGACAGAAGCGGCGAGGAGCCG
GAGGAGATGCGAGCTTCCCGCTTTAACGCGGGTCGTGAGCTGCGTCACGGTTTGGACCGAAGACGAGTGT
TGCGAGACGAGGATTTCGAAGTTGATGAAGTGACAGGGATCAGTCCTGCCAGGGCACACGTGGCTGCAGC
CAACCTTGTATCGGCTTACGAGCAGACAGTAAAGGAAGAGCGTAACTTCCAAAAGTCTTTTAATAATCAT
GTGCGAACCCTGATTGCCCGCGAAGAAGTTACCCTTGGTTTGATGCATTTGTGGGATTTGATGGAAGCTA
TCATTCAGAACCCTACTAGCAAACCTCTGACCGCCCAGCTGTTTCTGGTGGTGCAACACAGCAGAGACAA
TGAGGCTTTCAGAGAGGCGCTGCTGAACATCACCGAACCCGAGGGGAGATGGTTGTATGATCTTATCAAC
ATTCTACAGAGTATCATAGTGCAGGAGCGGAGCCTGGGCCTGGCCGAGAAGGTAGCTGCCATCAATTACT
CGGTTTTGAGCTTGGGAAAATATTACGCTCGCAAAATCTACAAGACTCCATACGTTCCCATAGACAAGGA
GGTGAAGATAGATGGGTTCTACATGCGCATGACGCTCAAGGTCTTGACCCTGAGCGATGATCTTGGGGTG
TATCGCAATGACAGAATGCATCGCGCGGTTAGCGCCAGCAGGAGGCGCGAGTTAAGCGACAGGGAACTGA
TGCACAGTTTGCAAAGAGCTCTGACTGGAGCTGGAACCGAGGGTGAGAATTACTTCGACATGGGAGCTGA
CTTGCAGTGGCAGCCTAGTCGCAGGGCTCTGAGCGCCGCGACGGCAGGATGTGAGCTTCCTTACATAGAA
GAGGCGGATGAAGGCGAGGAGGAAGAGGGCGAGTACTTGGAAGACTGATGGCACAACCCGTGTTTTTTGC
TAGATGGAACAGCAAGCACCGGATCCCGCAATGCGGGCGGCGCTGCAGAGCCAGCCGTCCGGCATTAACT
CCTCGGACGATTGGACCCAGGCCATGCAACGTATCATGGCGTTGACGACTCGCAACCCCGAAGCCTTTAG
ACAGCAACCCCAGGCCAACCGTCTATCGGCCATCATGGAAGCTGTAGTGCCTTCCCGATCTAATCCCACT
CATGAGAAGGTCCTGGCCATCGTGAACGCGTTGGTGGAGAACAAAGCTATTCGTCCAGATGAGGCCGGAC
TGGTATACAACGCTCTCTTAGAACGCGTGGCTCGCTACAACAGTAGCAATGTGCAAACCAATTTGGACCG
TATGATAACAGATGTACGCGAAGCCGTGTCTCAGCGCGAAAGGTTCCAGCGTGATGCCAACCTGGGTTCG
CTGGTGGCGTTAAATGCTTTCTTGAGTACTCAGCCTGCTAATGTGCCGCGTGGTCAACAGGATTATACTA
ACTTTTTAAGTGCTTTGAGACTGATGGTATCAGAAGTACCTCAGAGCGAAGTGTATCAGTCCGGTCCTGA
TTACTTCTTTCAGACTAGCAGACAGGGCTTGCAGACGGTAAATCTGAGCCAAGCTTTTAAAAACCTTAAA
GGTTTGTGGGGAGTGCATGCCCCGGTAGGAGAAAGAGCAACCGTGTCTAGCTTGTTAACTCCGAACTCCC
GCCTGTTATTACTGTTGGTAGCTCCTTTCACCGACAGCGGTAGCATCGACCGTAATTCCTATTTGGGTTA
CCTACTAAACCTGTATCGCGAAGCCATAGGGCAAAGTCAGGTGGACGAGCAGACCTATCAAGAAATTACC
CAAGTCAGTCGCGCTTTGGGACAGGAAGACACTGGCAGTTTGGAAGCCACTCTGAACTTCTTGCTTACCA
ATCGGTCTCAAAAGATCCCTCCTCAATATGCTCTTACTGCGGAGGAGGAGAGGATCCTTAGATATGTGCA
GCAGAGCGTGGGATTGTTTCTGATGCAAGAGGGGGCAACTCCGACTGCAGCACTGGACATGACAGCGCGA
AATATGGAGCCCAGCATGTATGCCAGTAACCGACCTTTCATTAACAAACTGCTGGACTACTTGCACAGAG
CTGCCGCTATGAACTCTGATTATTTCACCAATGCCATCTTAAACCCGCACTGGCTGCCCCCACCTGGTTT
CTACACGGGCGAATATGACATGCCCGACCCTAATGACGGATTTCTGTGGGACGACGTGGACAGCGATGTT
TTTTCACCTCTTTCTGATCATCGCACGTGGAAAAAGGAAGGCGGTGATAGAATGCATTCTTCTGCATCGC
TGTCCGGGGTCATGGGTGCTACCGCGGCTGAGCCCGAGTCTGCAAGTCCTTTTCCTAGTCTACCCTTTTC
TCTACACAGTGTACGTAGCAGCGAAGTGGGTAGAATAAGTCGCCCGAGTTTAATGGGCGAAGAGGAGTAC
CTAAACGATTCCTTGCTCAGACCGGCAAGAGAAAAAAATTTCCCAAACAATGGAATAGAAAGTTTGGTGG
ATAAAATGAGTAGATGGAAGACTTATGCTCAGGATCACAGAGACGAGCCTGGGATCATGGGGACTACAAG
TAGAGCGAGCCGTAGACGCCAGCGCCATGACAGACAGAGGGGTCTTGTGTGGGACGATGAGGATTCGGCC
GATGATAGCAGCGTGTTGGACTTGGGTGGGAGAGGAAGGGGCAACCCGTTTGCTCATTTGCGCCCTCGCT
TGGGTGGTATGTTGTGAAAAAAAATAAAAAAGAAAAACTCACCAAGGCCATGGCGACGAGCGTACGTTCG
TTCTTCTTTATTATCTGTGTCTAGTATAATGAGGCGAGTCGTGCTAGGCGGAGCGGTGGTGTATCCGGAG
GGTCCTCCTCCTTCGTACGAGAGCGTGATGCAGCAGCAGCAGGCGACGGCGGTGATGCAATCCCCACTGG
AGGCTCCCTTTGTGCCTCCGCGATACCTGGCACCTACGGAGGGCAGAAACAGCATTCGTTACTCGGAACT
GGCACCTCAGTACGATACCACCAGGTTGTATCTGGTGGACAACAAGTCGGCGGACATTGCTTCTCTGAAC
TATCAGAATGACCACAGCAACTTCTTGACCACGGTGGTGCAGAACAATGACTTTACCCCTACGGAAGCCA
GCACCCAGACCATTAACTTTGATGAACGATCGCGGTGGGGCGGTCAGCTAAAGACCATCATGCATACTAA
CATGCCAAACGTGAACGAGTATATGTTTAGTAACAAGTTCAAAGCGCGTGTGATGGTGTCCAGAAAACCT
CCCGACGGTGCTGCAGTTGGGGATACTTATGATCACAAGCAGGATATTTTGGAATATGAGTGGTTCGAGT
TTACTTTGCCAGAAGGCAACTTTTCAGTTACTATGACTATTGATTTGATGAACAATGCCATCATAGATAA
TTACTTGAAAGTGGGTAGACAGAATGGAGTGCTTGAAAGTGACATTGGTGTTAAGTTCGACACCAGGAAC
TTCAAGCTGGGATGGGATCCCGAAACCAAGTTGATCATGCCTGGAGTGTATACGTATGAAGCCTTCCATC
CTGACATTGTCTTACTGCCTGGCTGCGGAGTGGATTTTACCGAGAGTCGTTTGAGCAACCTTCTTGGTAT
CAGAAAAAAACAGCCATTTCAAGAGGGTTTTAAGATTTTGTATGAAGATTTAGAAGGTGGTAATATTCCG
GCCCTCTTGGATGTAGATGCCTATGAGAACAGTAAGAAAGAACAAAAAGCCAAAATAGAAGCTGCTACAG
CTGCTGCAGAAGCTAAGGCAAACATAGTTGCCAGCGACTCTACAAGGGTTGCTAACGCTGGAGAGGTCAG
AGGAGACAATTTTGCGCCAACACCTGTTCCGACTGCAGAATCATTATTGGCCGATGTGTCTGAAGGAACG
GACGTGAAACTCACTATTCAACCTGTAGAAAAAGATAGTAAGAATAGAAGCTATAATGTGTTGGAAGACA
AAATCAACACAGCCTATCGCAGTTGGTATCTTTCGTACAATTATGGCGATCCCGAAAAAGGAGTGCGTTC
CTGGACATTGCTCACCACCTCAGATGTCACCTGCGGAGCAGAGCAGGTTTACTGGTCGCTTCCAGACATG
ATGAAGGATCCTGTCACTTTCCGCTCCACTAGACAAGTCAGTAACTACCCTGTGGTGGGTGCAGAGCTTA
TGCCCGTCTTCTCAAAGAGCTTCTACAACGAACAAGCTGTGTACTCCCAGCAGCTCCGCCAGTCCACCTC
GCTTACGCACGTCTTCAACCGCTTTCCTGAGAACCAGATTTTAATCCGTCCGCCGGCGCCCACCATTACC
ACCGTCAGTGAAAACGTTCCTGCTCTCACAGATCACGGGACCCTGCCGTTGCGCAGCAGTATCCGGGGAG
TCCAACGTGTGACCGTTACTGACGCCAGACGCCGCACCTGTCCCTACGTGTACAAGGCACTGGGCATAGT
CGCACCGCGCGTCCTTTCAAGCCGCACTTTCTAAAAAAAAAATGTCCATTCTTATCTCGCCCAGTAATAA
CACCGGTTGGGGTCTGCGCGCTCCAAGCAAGATGTACGGAGGCGCACGCAAACGTTCTACCCAACATCCC
GTGCGTGTTCGCGGACATTTTCGCGCTCCATGGGGTGCCCTCAAGGGCCGCACTCGCGTTCGAACCACCG
TCGATGATGTAATCGATCAGGTGGTTGCCGACGCCCGTAATTATACTCCTACTGCGCCTACATCTACTGT
GGATGCAGTTATTGACAGTGTAGTGGCTGACGCTCGCAACTATGCTCGACGTAAGAGCCGGCGAAGGCGC
ATTGCCAGACGCCACCGAGCTACCACTGCCATGCGAGCCGCAAGAGCTCTGCTACGAAGAGCTAGACGCG
TGGGGCGAAGAGCCATGCTTAGGGCGGCCAGACGTGCAGCTTCGGGCGCCAGCGCCGGCAGGTCCCGCAG
GCAAGCAGCCGCTGTCGCAGCGGCGACTATTGCCGACATGGCCCAATCGCGAAGAGGCAATGTATACTGG
GTGCGTGACGCTGCCACCGGTCAACGTGTACCCGTGCGCACCCGTCCCCCTCGCACTTAGAAGATACTGA
GCAGTCTCCGATGTTGTGTCCCAGCGGCGAGGATGTCCAAGCGCAAATACAAGGAAGAAATGCTGCAGGT
TATCGCACCTGAAGTCTACGGCCAACCGTTGAAGGATGAAAAAAAACCCCGCAAAATCAAGCGGGTTAAA
AAGGACAAAAAAGAAGAGGAAGATGGCGATGATGGGCTGGCGGAGTTTGTGCGCGAGTTTGCCCCACGGC
GACGCGTGCAATGGCGTGGGCGCAAAGTTCGACATGTGTTGAGACCTGGAACTTCGGTGGTCTTTACACC
CGGCGAGCGTTCAAGCGCTACTTTTAAGCGTTCCTATGATGAGGTGTACGGGGATGATGATATTCTTGAG
CAGGCGGCTGACCGATTAGGCGAGTTTGCTTATGGCAAGCGTAGTAGAATAACTTCCAAGGATGAGACAG
TGTCAATACCCTTGGATCATGGAAATCCCACCCCTAGTCTTAAACCGGTCACTTTGCAGCAAGTGTTACC
CGTAACTCCGCGAACAGGTGTTAAACGCGAAGGTGAAGATTTGTATCCCACTATGCAACTGATGGTACCC
AAACGCCAGAAGTTGGAGGACGTTTTGGAGAAAGTAAAAGTGGATCCAGATATTCAACCTGAGGTTAAAG
TGAGACCCATTAAGCAGGTAGCGCCTGGTCTGGGGGTACAAACTGTAGACATTAAGATTCCCACTGAAAG
TATGGAAGTGCAAACTGAACCCGCAAAGCCTACTGCCACCTCCACTGAAGTGCAAACGGATCCATGGATG
CCCATGCCTATTACAACTGACGCCGCCGGTCCCACTCGAAGATCCCGACGAAAGTACGGTCCAGCAAGTC
TGTTGATGCCCAATTATGTTGTACACCCATCTATTATTCCTACTCCTGGTTACCGAGGCACTCGCTACTA
TCGCAGCCGAAACAGTACCTCCCGCCGTCGCCGCAAGACACCTGCAAATCGCAGTCGTCGCCGTAGACGC
ACAAGCAAACCGACTCCCGGCGCCCTGGTGCGGCAAGTGTACCGCAATGGTAGTGCGGAACCTTTGACAC
TGCCGCGTGCGCGTTACCATCCGAGTATCATCACTTAATCAATGTTGCCGCTGCCTCCTTGCAGATATGG
CCCTCACTTGTCGCCTTCGCGTTCCCATCACTGGTTACCGAGGAAGAAACTCGCGCCGTAGAAGAGGGAT
GTTGGGACGCGGAATGCGACGCTACAGGCGACGGCGTGCTATCCGCAAGCAATTGCGGGGTGGTTTTTTA
CCAGCCTTAATTCCAATTATCGCTGCTGCAATTGGCGCGATACCAGGCATAGCTTCCGTGGCGGTTCAGG
CCTCGCAACGACATTGACATTGGAAAAAAAACGTATAAATAAAAAAAAATACAATGGACTCTGACACTCC
TGGTCCTGTGACTATGTTTTCTTAGAGATGGAAGACATCAATTTTTCATCCTTGGCTCCGCGACACGGCA
CGAAGCCGTACATGGGCACCTGGAGCGACATCGGCACGAGCCAACTGAACGGGGGCGCCTTCAATTGGAG
CAGTATCTGGAGCGGGCTTAAAAATTTTGGCTCAACCATAAAAACATACGGGAACAAAGCTTGGAACAGC
AGTACAGGACAGGCGCTTAGAAATAAACTTAAAGACCAGAACTTCCAACAAAAAGTAGTCGATGGGATAG
CTTCCGGCATCAATGGAGTGGTAGATTTGGCTAACCAGGCTGTGCAGAAAAAGATAAACAGTCGTTTGGA
CCCGCCGCCAGCAACCCCAGGTGAAATGCAAGTGGAGGAAGAAATTCCTCCGCCAGAAAAACGAGGCGAC
AAGCGTCCGCGTCCCGATTTGGAAGAGACGCTGGTGACGCGCGTAGATGAACCGCCTTCTTATGAGGAAG
CAACGAAGCTTGGAATGCCCACCACTAGACCGATAGCCCCAATGGCCACCGGGGTGATGAAACCTTCTCA
GTTGCATCGACCCGTCACCTTGGATTTGCCCCCTCCCCCTGCTGCTACTGCTGTACCCGCTTCTAAGCCT
GTCGCTGCCCCGAAACCAGTCGCCGTAGCCAGGTCACGTCCCGGGGGCGCTCCTCGTCCAAATGCGCACT
GGCAAAATACTCTGAACAGCATCGTGGGTCTAGGCGTGCAAAGTGTAAAACGCCGTCGCTGCTTTTAATT
AAATATGGAGTAGCGCTTAACTTGCCTATCTGTGTATATGTGTCATTACACGCCGTCACAGCAGCAGAGG
AAAAAAGGAAGAGGTCGTGCGTCGACGCTGAGTTACTTTCAAGATGGCCACCCCATCGATGCTGCCCCAA
TGGGCATACATGCACATCGCCGGACAGGATGCTTCGGAGTACCTGAGTCCGGGTCTGGTGCAGTTCGCCC
GCGCCACAGACACCTACTTCAATCTGGGAAATAAGTTTAGAAATCCCACCGTAGCGCCGACCCACGATGT
GACCACCGACCGTAGCCAGCGGCTCATGTTGCGCTTCGTGCCCGTTGACCGGGAGGACAATACATACTCT
TACAAAGTGCGGTACACCCTGGCCGTGGGCGACAACAGAGTGCTGGATATGGCCAGCACGTTCTTTGACA
TTAGGGGCGTGTTGGACAGAGGTCCCAGTTTCAAACCCTATTCTGGTACGGCTTACAACTCTCTGGCTCC
TAAAGGCGCTCCAAATGCATCTCAATGGATTGCAAAAGGCGTACCAACTGCAGCAGCCGCAGGCAATGGT
GAAGAAGAACATGAAACAGAGGAGAAAACTGCTACTTACACTTTTGCCAATGCTCCTGTAAAAGCCGAGG
CTCAAATTACAAAAGAGGGCTTACCAATAGGTTTGGAGATTTCAGCTGAAAACGAATCTAAACCCATCTA
TGCAGATAAACTTTATCAGCCAGAACCTCAAGTGGGAGATGAAACTTGGACTGACCTAGACGGAAAAACC
GAAGAGTATGGAGGCAGGGCTCTAAAGCCTACTACTAACATGAAACCCTGTTACGGGTCCTATGCGAAGC
CTACTAATTTAAAAGGTGGTCAGGCAAAACCGAAAAACTCGGAACCGTCGAGTGAAAAAATTGAATATGA
TATTGACATGGAATTTTTTGATAACTCATCGCAAAGAACAAACTTCAGTCCTAAAATTGTCATGTATGCA
GAAAATGTAGGTTTGGAAACGCCAGACACTCATGTAGTGTACAAACCTGGAACAGAAGACACAAGTTCCG
AAGCTAATTTGGGACAACAGTCTATGCCCAACAGACCCAACTACATTGGCTTCAGAGATAACTTTATTGG
ACTCATGTACTATAACAGTACTGGTAACATGGGGGTGCTGGCTGGTCAAGCGTCTCAGTTAAATGCAGTG
GTTGACTTGCAGGACAGAAACACAGAACTTTCTTACCAACTCTTGCTTGACTCTCTGGGCGACAGAACCA
GATACTTTAGCATGTGGAATCAGGCTGTGGACAGTTATGATCCTGATGTACGTGTTATTGAAAATCATGG
TGTGGAAGATGAACTTCCCAACTATTGTTTTCCACTGGACGGCATAGGTGTTCCAACAACCAGTTACAAA
TCAATAGTTCCAAATGGAGAAGATAATAATAATTGGAAAGAACCTGAAGTAAATGGAACAAGTGAGATCG
GACAGGGTAATTTGTTTGCCATGGAAATTAACCTTCAAGCCAATCTATGGCGAAGTTTCCTTTATTCCAA
TGTGGCTCTGTATCTCCCAGACTCGTACAAATACACCCCGTCCAATGTCACTCTTCCAGAAAACAAAAAC
ACCTACGACTACATGAACGGGCGGGTGGTGCCGCCATCTCTAGTAGACACCTATGTGAACATTGGTGCCA
GGTGGTCTCTGGATGCCATGGACAATGTCAACCCATTCAACCACCACCGTAACGCTGGCTTGCGTTACCG
ATCTATGCTTCTGGGTAACGGACGTTATGTGCCTTTCCACATACAAGTGCCTCAAAAATTCTTCGCTGTT
AAAAACCTGCTGCTTCTCCCAGGCTCCTACACTTATGAGTGGAACTTTAGGAAGGATGTGAACATGGTTC
TACAGAGTTCCCTCGGTAACGACCTGCGGGTAGATGGCGCCAGCATCAGTTTCACGAGCATCAACCTCTA
TGCTACTTTTTTCCCCATGGCTCACAACACCGCTTCCACCCTTGAAGCCATGCTGCGGAATGACACCAAT
GATCAGTCATTCAACGACTACCTATCTGCAGCTAACATGCTCTACCCCATTCCTGCCAATGCAACCAATA
TTCCCATTTCCATTCCTTCTCGCAACTGGGCGGCTTTCAGAGGCTGGTCATTTACCAGACTGAAAACCAA
AGAAACTCCCTCTTTGGGGTCTGGATTTGACCCCTACTTTGTCTATTCTGGTTCTATTCCCTACCTGGAT
GGTACCTTCTACCTGAACCACACTTTTAAGAAGGTTTCCATCATGTTTGACTCTTCAGTGAGCTGGCCTG
GAAATGACAGGTTACTATCTCCTAACGAATTTGAAATAAAGCGCACTGTGGATGGCGAAGGCTACAACGT
AGCCCAATGCAACATGACCAAAGACTGGTTCTTGGTACAGATGCTCGCCAACTACAACATCGGCTATCAG
GGCTTCTACATTCCAGAAGGATACAAAGATCGCATGTATTCATTTTTCAGAAACTTCCAGCCCATGAGCA
GGCAGGTGGTTGATGAGGTCAATTACAAAGACTTCAAGGCCGTCGCCATACCCTACCAACACAACAACTC
TGGCTTTGTGGGTTACATGGCTCCGACCATGCGCCAAGGTCAACCCTATCCCGCTAACTATCCCTATCCA
CTCATTGGAACAACTGCCGTAAATAGTGTTACGCAGAAAAAGTTCTTGTGTGACAGAACCATGTGGCGCA
TACCGTTCTCGAGCAACTTCATGTCTATGGGGGCCCTTACAGACTTGGGACAGAATATGCTCTATGCCAA
CTCAGCTCATGCTCTGGACATGACCTTTGAGGTGGATCCCATGGATGAGCCCACCCTGCTTTATCTTCTC
TTCGAAGTTTTCGACGTGGTCAGAGTGCATCAGCCACACCGCGGCATCATCGAGGCAGTCTACCTGCGTA
CACCGTTCTCGGCCGGTAACGCTACCACGTAAGAAGCTTCTTGCTTCTTGCAAATAGCAGCTGCAACCAT
GGCCTGCGGATCCCAAAACGGCTCCAGCGAGCAAGAGCTCAGAGCCATTGTCCAAGACCTGGGTTGCGGA
CCCTATTTTTTGGGAACCTACGATAAGCGCTTCCCGGGGTTCATGGCCCCCGATAAGCTCGCCTGTGCCA
TTGTAAATACGGCCGGACGTGAGACGGGGGGAGAGCACTGGTTGGCTTTCGGTTGGAACCCACGTTCTAA
CACCTGCTACCTTTTTGATCCTTTTGGATTCTCGGATGATCGTCTCAAACAGATTTACCAGTTTGAATAT
GAGGGTCTCCTGCGCCGCAGCGCTCTTGCTACCAAGGACCGCTGTATTACGCTGGAAAAATCTACCCAGA
CCGTGCAGGGCCCCCGTTCTGCCGCCTGCGGACTTTTCTGCTGCATGTTCCTTCACGCCTTTGTGCACTG
GCCTGACCGTCCCATGGACGGAAACCCCACCATGAAATTGCTAACTGGAGTGCCAAACAACATGCTTCAT
TCTCCTAAAGTCCAGCCCACCCTGTGTGACAATCAAAAAGCACTCTACCATTTTCTTAATACCCATTCGC
CTTATTTTCGCTCTCATCGTACACACATCGAAAGGGCCACTGCGTTCGACCGTATGGATGTTCAATAATG
ACTCATGTAAACAACGTGTTCAATAAACATCACTTTATTTTTTTACATGTATCAAGGCTCTGGATTACTT
ATTTATTTACAAGTCGAATGGGTTCTGACGAGAATCAGAATGACCCGCAGGCAGTGATACGTTGCGGAAC
TGATACTTGGGTTGCCACTTGAATTCGGGAATCACCAACTTGGGAACCGGTATATCGGGCAGGATGTCAC
TCCACAGCTTTCTGGTCAGCTGCAAAGCTCCAAGCAGGTCAGGAGCCGAAATCTTGAAATCACAATTAGG
ACCAGTGCTCTGAGCGCGAGAGTTGCGGTACACCGGATTGCAGCACTGAAACACCATCAGCGACGGATGT
CTCACGCTTGCCAGCACGGTGGGATCTGCAATCATGCCCACATCCAGATCTTCAGCATTGGCAATGCTGA
ACGGGGTCATCTTGCAGGTCTGCCTACCCATGGCGGGCACCCAATTAGGCTTGTGGTTGCAATCGCAGTG
CAGGGGGATCAGTATCATCTTGGCCTGATCCTGTCTGATTCCTGGATACACGGCTCTCATGAAAGCATCA
TATTGCTTGAAAGCCTGCTGGGCTTTACTACCCTCGGTATAAAACATCCCGCAGGACCTGCTCGAAAACT
GGTTAGCTGCACAGCCGGCATCATTCACACAGCAGCGGGCGTCATTGTTGGCTATTTGCACCACACTTCT
GCCCCAGCGGTTTTGGGTGATTTTGGTTCGCTCGGGATTCTCCTTTAAGGCTCGTTGTCCGTTCTCGCTG
GCCACATCCATCTCGATAATCTGCTCCTTCTGAATCATAATATTGCCATGCAGGCACTTCAGCTTGCCCT
CATAATCATTGCAGCCATGAGGCCACAACGCACAGCCTGTACATTCCCAATTATGGTGGGCGATCTGAGA
AAAAGAATGTATCATTCCCTGCAGAAATCTTCCCATCATCGTGCTCAGTGTCTTGTGACTAGTGAAAGTT
AACTGGATGCCTCGGTGCTCTTCGTTTACGTACTGGTGACAGATGCGCTTGTATTGTTCGTGTTGCTCAG
GCATTAGTTTAAAACAGGTTCTAAGTTCGTTATCCAGCCTGTACTTCTCCATCAGCAGACACATCACTTC
CATGCCTTTCTCCCAAGCAGACACCAGGGGCAAGCTAATCGGATTCTTAACAGTGCAGGCAGCAGCTCCT
TTAGCCAGAGGGTCATCTTTAGCGATCTTCTCAATGCTTCTTTTGCCATCCTTCTCAACGATGCGCACGG
GCGGGTAGCTGAAACCCACTGCTACAAGTTGCGCCTCTTCTCTTTCTTCTTCGCTGTCTTGACTGATGTC
TTGCATGGGGATATGTTTGGTCTTCCTTGGCTTCTTTTTGGGGGGTATCGGAGGAGGAGGACTGTCGCTC
CGTTCCGGAGACAGGGAGGATTGTGACGTTTCGCTCACCATTACCAACTGACTGTCGGTAGAAGAACCTG
ACCCCACACGGCGACAGGTGTTTTTCTTCGGGGGCAGAGGTGGAGGCGATTGCGAAGGGCTGCGGTCCGA
CCTGGAAGGCGGATGACTGGCAGAACCCCTTCCGCGTTCGGGGGTGTGCTCCCTGTGGCGGTCGCTTAAC
TGATTTCCTTCGCGGCTGGCCATTGTGTTCTCCTAGGCAGAGAAACAACAGACATGGAAACTCAGCCATT
GCTGTCAACATCGCCACGAGTGCCATCACATCTCGTCCTCAGCGACGAGGAAAAGGAGCAGAGCTTAAGC
ATTCCACCGCCCAGTCCTGCCACCACCTCTACCCTAGAAGATAAGGAGGTCGACGCATCTCATGACATGC
AGAATAAAAAAGCGAAAGAGTCTGAGACAGACATCGAGCAAGACCCGGGCTATGTGACACCGGTGGAACA
CGAGGAAGAGTTGAAACGCTTTCTAGAGAGAGAGGATGAAAACTGCCCAAAACAGCGAGCAGATAACTAT
CACCAAGATGCTGGAAATAGGGATCAGAACACCGACTACCTCATAGGGCTTGACGGGGAAGACGCGCTCC
TTAAACATCTAGCAAGACAGTCGCTCATAGTCAAGGATGCATTATTGGACAGAACTGAAGTGCCCATCAG
TGTGGAAGAGCTCAGCTGCGCCTACGAGCTTAACCTTTTTTCACCTCGTACTCCCCCCAAACGTCAGCCA
AACGGCACCTGCGAGCCAAATCCTCGCTTAAACTTTTATCCAGCTTTTGCTGTGCCAGAAGTACTGGCTA
CCTATCACATCTTTTTTAAAAATCAAAAAATTCCAGTCTCCTGCCGCGCTAATCGCACCCGCGCCGATGC
CCTACTCAATCTGGGACCTGGTTCACGCTTACCTGATATAGCTTCCTTGGAAGAGGTTCCAAAGATCTTC
GAGGGTCTGGGCAATAATGAGACTCGGGCCGCAAATGCTCTGCAAAAGGGAGAAAATGGCATGGATGAGC
ATCACAGCGTTCTGGTGGAATTGGAAGGCGATAATGCCAGACTCGCAGTACTCAAGCGAAGCGTCGAGGT
CACACACTTCGCATATCCCGCTGTCAACCTGCCCCCTAAAGTCATGACGGCGGTCATGGACCAGTTACTC
ATTAAGCGCGCAAGTCCCCTTTCAGAAGACATGCATGACCCAGATGCCTGTGATGAGGGTAAACCAGTGG
TCAGTGATGAGCAGCTAACCCGATGGCTGGGCACCGACTCTCCCCGGGATTTGGAAGAGCGTCGCAAGCT
TATGATGGCCGTGGTGCTGGTTACCGTAGAACTAGAGTGTCTCCGACGTTTCTTTACCGATTCAGAAACC
TTGCGCAAACTCGAAGAGAATCTGCACTACACTTTTAGACACGGCTTTGTGCGGCAGGCATGCAAGATAT
CTAACGTGGAACTCACCAACCTGGTTTCCTACATGGGTATTCTGCATGAGAATCGCCTAGGACAAAGCGT
GCTGCACAGCACCCTTAAGGGGGAAGCCCGCCGTGATTACATCCGCGATTGTGTCTATCTCTACCTGTGC
CACACGTGGCAAACCGGCATGGGTGTATGGCAGCAATGTTTAGAAGAACAGAACTTGAAAGAGCTTGACA
AGCTCTTACAGAAATCTCTTAAGGTTCTGTGGACAGGGTTCGACGAGCGCACCGTCGCTTCCGACCTGGC
AGACCTCATCTTCCCAGAGCGTCTCAGGGTTACTTTGCGAAACGGATTGCCTGACTTTATGAGCCAGAGC
ATGCTTAACAATTTTCGCTCTTTCATCCTGGAACGCTCCGGTATCCTGCCCGCCACCTGCTGCGCACTGC
CCTCCGACTTTGTGCCTCTCACCTACCGCGAGTGCCCCCCGCCGCTATGGAGTCACTGCTACCTGTTCCG
TCTGGCCAACTATCTCTCCTACCACTCGGATGTGATCGAGGATGTGAGCGGAGACGGCTTGCTGGAGTGC
CACTGCCGCTGCAATCTGTGCACGCCCCACCGGTCCCTAGCTTGCAACCCCCAGTTGATGAGCGAAACCC
AGATAATAGGCACCTTTGAATTGCAAGGCCCCAGCAGCCAAGGCGATGGGTCTTCTCCTGGGCAAAGTTT
AAAACTGACCCCGGGACTGTGGACCTCCGCCTACTTGCGCAAGTTTGCTCCGGAAGATTACCACCCCTAT
GAAATCAAGTTCTATGAGGACCAATCACAGCCTCCAAAGGCCGAACTTTCGGCTTGCGTCATCACCCAGG
GGGCAATTCTGGCCCAATTGCAAGCCATCCAAAAATCCCGCCAAGAATTTCTACTGAAAAAGGGTAAGGG
GGTCTACCTTGACCCCCAGACCGGCGAGGAACTCAACACAAGGTTCCCTCAGGATGTCCCAACGACGAGA
AAACAAGAAGTTGAAGGTGCAGCCGCCGCCCCCAGAAGATATGGAGGAAGATTGGGACAGTCAGGCAGAG
GAGGCGGAGGAGGACAGTCTGGAGGACAGTCTGGAGGAAGACAGTTTGGAGGAGGAAAACGAGGAGGCAG
AGGAGGTGGAAGAAGTAACCGCCGACAAACAGTTATCCTCGGCTGCGGAGACAAGCAACAGCGCTACCAT
CTCCGCTCCGAGTCGAGGAACCCGGCGGCGTCCCAGCAGTAGATGGGACGAGACCGGACGCTTCCCGAAC
CCAACCAGCGCTTCCAAGACCGGTAAGAAGGATCGGCAGGGATACAAGTCCTGGCGGGGGCATAAGAATG
CCATCATCTCCTGCTTGCATGAGTGCGGGGGCAACATATCCTTCACGCGGCGCTACTTGCTATTCCACCA
TGGGGTGAACTTTCCGCGCAATGTTTTGCATTACTACCGTCACCTCCACAGCCCCTACTATAGCCAGCAA
ATCCCGACAGTCTCGACAGATAAAGACAGCGGCGGCGACCTCCAACAGAAAACCAGCAGCGGCAGTTAGA
AAATACACAACAAGTGCAGCAACAGGAGGATTAAAGATTACAGCCAACGAGCCAGCGCAAACCCGAGAGT
TAAGAAATCGGATCTTTCCAACCCTGTATGCCATCTTCCAGCAGAGTCGGGGTCAAGAGCAGGAACTGAA
AATAAAAAACCGATCTCTGCGTTCGCTCACCAGAAGTTGTTTGTATCACAAGAGCGAAGATCAACTTCAG
CGCACTCTCGAGGACGCCGAGGCTCTCTTCAACAAGTACTGCGCGCTGACTCTTAAAGAGTAGGCAGCGA
CCGCGCTTATTCAAAAAAGGCGGGAATTACATCATCCTCGACATGAGTAAAGAAATTCCCACGCCTTACA
TGTGGAGTTATCAACCCCAAATGGGATTGGCAGCAGGCGCCTCCCAGGACTACTCCACCCGCATGAATTG
GCTCAGCGCCGGGCCTTCTATGATTTCTCGAGTTAATGATATACGCGCCTACCGAAACCAAATACTTTTG
GAACAGTCAGCTCTTACCACCACGCCCCGCCAACACCTTAATCCCAGAAATTGGCCCGCCGCCCTAGTGT
ACCAGGAAAGTCCCGCTCCCACCACTGTATTACTTCCTCGAGACGCCCAGGCCGAAGTCCAAATGACTAA
TGCAGGTGCGCAGTTAGCTGGCGGCTCCACCCTATGTCGTCACAGGCCTCGGCATAATATAAAACGCCTG
ATGATCAGAGGCCGAGGTATCCAGCTCAACGACGAGTCGGTGAGCTCTCCGCTTGGTCTACGACCAGACG
GAATCTTTCAGATTGCCGGCTGCGGGAGATCTTCCTTCACCCCTCGTCAGGCTGTTCTGACTTTGGAAAG
TTCGTCTTCGCAACCCCGCTCGGGCGGAATCGGGACCGTTCAATTTGTAGAGGAGTTTACTCCCTCTGTC
TACTTCAACCCCTTCTCCGGATCTCCTGGGCACTACCCGGACGAGTTCATACCGAACTTCGACGCGATTA
GCGAGTCAGTGGACGGCTACGATTGATGTCTGGTGACGCGGCTGAGCTATCTCGGCTGCGACATCTAGAC
CACTGCCGCCGCTTTCGCTGCTTTGCCCGGGAACTTATTGAGTTCATCTACTTCGAACTCCCCAAGGATC
ACCCTCAAGGTCCGGCCCACGGAGTGCGGATTACTATCGAAGGCAAAATAGACTCTCGCCTGCAACGAAT
TTTCTCCCAGCGGCCCGTGCTGATCGAGCGAGACCAGGGAAACACCACGGTTAGTAATCAATTACGGGGT
CATTAGTTCATAGCCCATATATGGAGTTAACTTGTTTATTGCAGCTTATAATGGTTACAAATAAAGCAAT
AGCATCACAAATTTCACAAATAAAGCATTTTTTTCACTGCATTCTAGTTGTGGTTTGTCCAAACTCATCA
ATGTATCTTATCATGTCTGCTCGAAGCGGCCGGCCGCCCCGACTCTAGAGTCGCGGCCTCATTAGGAAGT
TCCTATACTTTCTAGAGAATAGGAACTTCTCAGAAGAACTCGTCAAGAAGGCGATAGAAGGCGATGCGCT
GCGAATCGGGAGCGGCGATACCGTAAAGCACGAGGAAGCGGTCAGCCCATTCGCCGCCAAGCTCTTCAGC
AATATCACGGGTAGCCAACGCTATGTCCTGATAGCGGTCCGCCACACCCAGCCGGCCACAGTCGATGAAT
CCAGAAAAGCGGCCATTTTCCACCATGATATTCGGCAAGCAGGCATCGCCATGGGTCACGACGAGATCCT
CGCCGTCGGGCATGCGCGCCTTGAGCCTGGCGAACAGTTCGGCTGGCGCGAGCCCCTGATGCTCTTCGTC
CAGATCATCCTGATCGACAAGACCGGCTTCCATCCGAGTACGTGCTCGCTCGATGCGATGTTTCGCTTGG
TGGTCGAATGGGCAGGTAGCCGGATCAAGCGTATGCAGCCGCCGCATTGCATCAGCCATGATGGATACTT
TCTCGGCAGGAGCAAGGTGAGATGACAGGAGATCCTGCCCCGGCACTTCGCCCAATAGCAGCCAGTCCCT
TCCCGCTTCAGTGACAACGTCGAGCACAGCTGCGCAAGGAACGCCCGTCGTGGCCAGCCACGATAGCCGC
GCTGCCTCGTCCTGCAGTTCATTCAGGGCACCGGACAGGTCGGTCTTGACAAAAAGAACCGGGCGCCCCT
GCGCTGACAGCCGGAACACGGCGGCATCAGAGCAGCCGATTGCCTGTTGTGCCCAGTCATAGCCGAATAG
CCTCTCCACCCAAGCGGCCGGAGAACCTGCGTGCAATCCATCTTGTTCAATGGCCGATCCCATAACACCC
CTTGTATTACTGTTTATGTAAGCAGACAGTTTTACTGTTCGTGATGATATATTTTTATCTTGTGCAATGT
AACAGGTTGTGGCCATAGCGGGCCCGGGATTTTCCTCCACGTCCCCGCATGTTAGAAGACTTCCCCTGCC
CTCGGCTCTGGAAGTTCCTATACTTTCTAGAGAATAGGAACTTCCCGCCAGAATGCGTTCGCACAGCCGC
CAGCCGGTCACTCCGTTGATGGTTACTCGGAACAGCAGGGAGCCGTCGGGGTTGATCAGGCGCTCGTCGA
TAATTTTGTTGCCGTTCCACAGGGTCCCTGTTACAGTGATCTTTTTGCCGTCGAACACGGCGATGCCTTC
ATACGGCCGTCCGAAATAGTCGATCATGTTCGGCGTAACCCCGTCGATTACCAGTGTGCCATAGTGCAGG
ATCACCTTAAAGTGATGATCATCCACAGGGTACACCACCTTAAAAATTTTTTCGATCTGGCCCATTTGGT
CGCCGCTCAGACCTTCATACGGGATGATGACATGGATGTCGATCTTCAGCCCATTTTCACCGCTCAGGAC
AATCCTTTGGATCGGAGTTACGGACACCCCGAGATTCTGAAACAAACTGGACACACCTCCCTGTTCAAGG
ACTTGGTCCAGGTTGTAGCCGGCTGTCTGTCGCCAGTCCCCAACGAAATCTTCGAGTGTGAAGACCATGG
ATCCGGGCCCGGGGTTTTCTTCAACGTCTCCAGCCTGCTTCAGCAGGCTGAAGTTAGTAGCTCCGCTTCC
TCGAGCTCGAGATCTGGCGAAGGCGATGGGGGTCTTGAAGGCGTGCTGGTACTCCACGATGCCCAGCTCG
GTGTTGCTGTGCAGCTCCTCCACGCGGCGGAAGGCGAACATGGGGCCCCCGTTCTGCAGGATGCTGGGGT
GGATGGCGCTCTTGAAGTGCATGTGGCTGTCCACCACGAAGCTGTAGTAGCCGCCGTCGCGCAGGCTGAA
GGTGCGGGCGAAGCTGCCCACCAGCACGTTATCGCCCATGGGGTGCAGGTGCTCCACGGTGGCGTTGCTG
CGGATGATCTTGTCGGTGAAGATCACGCTGTCCTCGGGGAAGCCGGTGCCCACCACCTTGAAGTCGCCGA
TCACGCGGCCGGCCTCGTAGCGGTAGCTGAAGCTCACGTGCAGCACGCCGCCGTCCTCGTACTTCTCGAT
GCGGGTGTTGGTGTAGCCGCCGTTGTTGATGGCGTGCAGGAAGGGGTTCTCGTAGCCGCTGGGGTAGGTG
CCGAAGTGGTAGAAGCCGTAGCCCATCACGTGGCTCAGCAGGTAGGGGCTGAAGGTCAGGGCGCCTTTGG
TGCTCTTCATCTTGTTGGTCATGCGGCCCTGCTCGGGGGTGCCCTCTCCGCCGCCCACCAGCTCGAACTC
CACGCCGTTCAGGGTGCCGGTGATGCGGCACTCGATCTTCATGGCGGGCATGGTGGCGACCGGTAGCGCT
AGCGGCTTCGGTACCACGCGTTCGCTCGAATTAATCAATTCTTTGCCAAAATGATGAGACAGCACAATAA
CCAGCACGTTGCCCAGGAGCTGTAGGAAAAAGAAGAAGGCATGAACATGGTTAGCAGAGGCTCTAGAGCC
GCCGGTCACACGCCAGAAGCCGAACCCCGCCCTGCCCCGTCCCCCCCGAAGGCAGCCGTCCCCCCGCGGA
CAGCCCCGAGGCTGGAGAGGGAGAAGGGGACGGCGGCGCGGCGACGCACGAAGGCCCTCCCCGCCCATTT
CCTTCCTGCCGGGGCCCTCCCGGAGCCCCTCAAGGCTTTCACGCAGCCACAGAAAAGAAACAAGCCGTCA
TTAAACCAAGCGCTAATTACAGCCCGGAGGAGAAGGGCCGTCCCGCCCGCTCACCTGTGGGAGTAACGCG
GTCAGTCAGAGCCGGGGCGGGCGGCGCGAGGCGGCGCGGAGCGGGGCACGGGGCGAAGGCAACGCAGCGA
CTCCCGCCCGCCGCGCGCTTCGCTTTTTATAGGGCCGCCGCCGCCGCCGCCTCGCCATAAAAGGAAACTT
TCGGAGCGCGCCGCTCTGATTGGCTGCCGCCGCACCTCTCCGCCTCGCCCCGCCCCGCCCCTCGCCCCGC
CCCGCCCCGCCTGGCGCGCGCCCCCCCCCCCCCCCCGCCCCCATCGCTGCACAAAATAATTAAAAAATAA
ATAAATACAAAATTGGGGGTGGGGAGGGGGGGGAGATGGGGAGAGTGAAGCAGAACGTGGGGCTCACCTC
GACCATGGTAATAGCGATGACTAATACGTAGATGTACTGCCAAGTAGGAAAGTCCCATAAGGTCATGTAC
TGGGCATAATGCCAGGCGGGCCATTTACCGTCATTGACGTCAATAGGGGGCGTACTTGGCATATGATACA
CTTGATGTACTGCCAAGTGGGCAGTTTACCGTAAATACTCCACCCATTGACGTCAATGGAAAGTCCCTAT
TGGCGTTACTATGGGAACATACGTCATTATTGACGTCAATGGGCGGGGGTCGTTGGGCGGTCAGCCAGGC
GGGCCATTTACCGTAAGTTATGTAACGCGGAACAACAACAATTGCATTCATTTTATGTTTCAGGTTCAGG
GGGAGGTGTGGTCCTGCGATTCCATCGAGTGCACCTACACCCTGCTGAAGACCCTATGCGGCCTAAGAGA
CCTGCTACCAATGAATTAAAAAAAAATGATTAATAAAAAATCACTTACTTGAAATCAGCAATAAGGTCTC
TGTTGAAATTTTCTCCCAGCAGCACCTCACTTCCCTCTTCCCAACTCTGGTATTCTAAACCCCGTTCAGC
GGCATACTTTCTCCATACTTTAAAGGGGATGTCAAATTTTAGCTCCTCTCCTGTACCCACAATCTTCATG
TCTTTCTTCCCAGATGACCAAGAGAGTCCGGCTCAGTGACTCCTTCAACCCTGTCTACCCCTATGAAGAT
GAAAGCACCTCCCAACACCCCTTTATAAACCCAGGGTTTATTTCCCCAAATGGCTTCACACAAAGCCCAG
ACGGAGTTCTTACTTTAAAATGTTTAACCCCACTAACAACCACAGGCGGATCTCTACAGCTAAAAGTGGG
AGGGGGACTTACAGTGGATGACACTGATGGTACCTTACAAGAAAACATACGTGCTACAGCACCCATTACT
AAAAATAATCACTCTGTAGAACTATCCATTGGAAATGGATTAGAAACTCAAAACAATAAACTATGTGCCA
AATTGGGAAATGGGTTAAAATTTAACAACGGTGACATTTGTATAAAGGATAGTATTAACACCTTATGGAC
TGGAATAAACCCTCCACCTAACTGTCAAATTGTGGAAAACACTAATACAAATGATGGCAAACTTACTTTA
GTATTAGTAAAAAATGGAGGGCTTGTTAATGGCTACGTGTCTCTAGTTGGTGTATCAGACACTGTGAACC
AAATGTTCACACAAAAGACAGCAAACATCCAATTAAGATTATATTTTGACTCTTCTGGAAATCTATTAAC
TGAGGAATCAGACTTAAAAATTCCACTTAAAAATAAATCTTCTACAGCGACCAGTGAAACTGTAGCCAGC
AGCAAAGCCTTTATGCCAAGTACTACAGCTTATCCCTTCAACACCACTACTAGGGATAGTGAAAACTACA
TTCATGGAATATGTTACTACATGACTAGTTATGATAGAAGTCTATTTCCCTTGAACATTTCTATAATGCT
AAACAGCCGTATGATTTCTTCCAATGTTGCCTATGCCATACAATTTGAATGGAATCTAAATGCAAGTGAA
TCTCCAGAAAGCAACATAGCTACGCTGACCACATCCCCCTTTTTCTTTTCTTACATTACAGAAGACGACA
ACTAAAATAAAGTTTAAGTGTTTTTATTTAAAATCACAAAATTCGAGTAGTTATTTTGCCTCCACCTTCC
CATTTGACAGAATACACAGTCCTTTCTCCCCGGCTGGCCTTAAAAAGCATCATATCATGGGTAACAGACA
TATTCTTAGGTGTTATATTCCACACGGTTTCCTGTCGAGCCAAACGCTCATCAGTGATATTAATAAACTC
CCCGGGCAGCTCACTTAAGTTCATGTCGCTGTCCAGCTGCTGAGCCACAGGCTGCTGTCCAACTTGCGGT
TGCTTAACGGGCGGCGAAGGAGAAGTCCACGCCTACATGGGGGTAGAGTCATAATCGTGCATCAGGATAG
GGCGGTGGTGCTGCAGCAGCGCGCGAATAAACTGCTGCCGCCGCCGCTCCGTCCTGCAGGAATACAACAT
GGCAGTGGTCTCCTCAGCGATGATTCGCACCGCCCGCAGCATAAGGCGCCTTGTCCTCCGGGCACAGCAG
CGCACCCTGATCTCACTTAAATCAGCACAGTAACTGCAGCACAGCACCACAATATTGTTCAAAATCCCAC
AGTGCAAGGCGCTGTATCCAAAGCTCATGGCGGGGACCACAGAACCCACGTGGCCATCATACCACAAGCG
CAGGTAGATTAAGTGGCGACCCCTCATAAACACGCTGGACATAAACATTACCTCTTTTGGCATGTTGTAA
TTCACCACCTCCCGGTACCATATAAACCTCTGATTAAACATGGCGCCATCCACCACCATCCTAAACCAGC
TGGCCAAAACCTGCCCGCCGGCTATACACTGCAGGGAACCGGGACTGGAACAATGACAGTGGAGAGCCCA
GGACTCGTAACCATGGATCATCATGCTCGTCATGATATCAATGTTGGCACAACACAGGCACACGTGCATA
CACTTCCTCAGGATTACAAGCTCCTCCCGCGTTAGAACCATATCCCAGGGAACAACCCATTCCTGAATCA
GCGTAAATCCCACACTGCAGGGAAGACCTCGCACGTAACTCACGTTGTGCATTGTCAAAGTGTTACATTC
GGGCAGCAGCGGATGATCCTCCAGTATGGTAGCGCGGGTTTCTGTCTCAAAAGGAGGTAGACGATCCCTA
CTGTACGGAGTGCGCCGAGACAACCGAGATCGTGTTGGTCGTAGTGTCATGCCAAATGGAACGCCGGACG
TAGTCATTCTCGTATTTTGTATAGCAAAACGCGGCCCTGGCAGAACACACTCTTCTTCGCCTTCTATCCT
GCCGCTTAGCGTGTTCCGTGTGATAGTTCAAGTACAGCCACACTCTTAAGTTGGTCAAAAGAATGCTGGC
TTCAGTTGTAATCAAAACTCCATCGCATCTAATTGTTCTGAGGAAATCATCCACGGTAGCATATGCAAAT
CCCAACCAAGCAATGCAACTGGATTGCGTTTCAAGCAGGAGAGGAGAGGGAAGAGACGGAAGAACCATGT
TAATTTTTATTCCAAACGATCTCGCAGTACTTCAAATTGTAGATCGCGCAGATGGCATCTCTCGCCCCCA
CTGTGTTGGTGAAAAAGCACAGCTAAATCAAAAGAAATGCGATTTTCAAGGTGCTCAACGGTGGCTTCCA
ACAAAGCCTCCACGCGCACATCCAAGAACAAAAGAATACCAAAAGAAGGAGCATTTTCTAACTCCTCAAT
CATCATATTACATTCCTGCACCATTCCCAGATAATTTTCAGCTTTCCAGCCTTGAATTATTCGTGTCAGT
TCTTGTGGTAAATCCAATCCACACATTACAAACAGGTCCCGGAGGGCGCCCTCCACCACCATTCTTAAAC
ACACCCTCATAATGACAAAATATCTTGCTCCTGTGTCACCTGTAGCGAATTGAGAATGGCAACATCAATT
GACATGCCCTTGGCTCTAAGTTCTTCTTTAAGTTCTAGTTGTAAAAACTCTCTCATATTATCACCAAACT
GCTTAGCCAGAAGCCCCCCGGGAACAAGAGCAGGGGACGCTACAGTGCAGTACAAGCGCAGACCTCCCCA
ATTGGCTCCAGCAAAAACAAGATTGGAATAAGCATATTGGGAACCACCAGTAATATCATCGAAGTTGCTG
GAAATATAATCAGGCAGAGTTTCTTGTAGAAATTGAATAAAAGAAAAATTTGCCAAAAAAACATTCAAAA
CCTCTGGGATGCAAATGCAATAGGTTACCGCGCTGCGCTCCAACATTGTTAGTTTTGAATTAGTCTGCAA
AAATAAAAAAAAAACAAGCGTCATATCATAGTAGCCTGACGAACAGGTGGATAAATCAGTCTTTCCATCA
CAAGACAAGCCACAGGGTCTCCAGCTCGACCCTCGTAAAACCTGTCATCGTGATTAAACAACAGCACCGA
AAGTTCCTCGCGGTGACCAGCATGAATAAGTCTTGATGAAGCATACAATCCAGACATGTTAGCATCAGTT
AAGGAGAAAAAACAGCCAACATAGCCTTTGGGTATAATTATGCTTAATCGTAAGTATAGCAAAGCCACCC
CTCGCGGATACAAAGTAAAAGGCACAGGAGAATAAAAAATATAATTATTTCTCTGCTGCTGTTTAGGCAA
CGTCGCCCCCGGTCCCTCTAAATACACATACAAAGCCTCATCAGCCATGGCTTACCAGAGAAAGTACAGC
GGGCACACAAACCACAAGCTCTAAAGTCACTCTCCAACCTCTCCACAATATATATACACAAGCCCTAAAC
TGACGTAATGGGACTAAAGTGTAAAAAATCCCGCCAAACCCAACACACACCCCGAAACTGCGTCACCAGG
GAAAAGTACAGTTTCACTTCCGCAATCCCAACAAGCGTCACTTCCTCTTTCTCACGGTACGTCACATCCC
ATTAACTTACAACGTCATTTTCCCACGGCCGCGCCGCCCCTTTTAACCGTTAACCCCACAGCCAATCACC
ACACGGCCCACACTTTTTAAAATCACCTCATTTACATATTGGCACCATTCCATCTATAAGGTATATTATT
GATGATGGCCAAGCTATTTAGGTGACACTATAGAATACTCAAGCTATGCATCAAGCTTGGTACCGAGCTC
GGATCCACTAGTAACGGCCGCCAGTGTGCTGGAATTCGCCCTTGTTTAAACGCGATCGCTTGAGATCGTT
TTGGTCTGCGCGTAATCTCTTGCTCTGAAAACGAAAAAACCGCCTTGCAGGGCGGTTTTTCGAAGGTTCT
CTGAGCTACCAACTCTTTGAACCGAGGTAACTGGCTTGGAGGAGCGCAGTCACCAAAACTTGTCCTTTCA
GTTTAGCCTTAACCGGCGCATGACTTCAAGACTAACTCCTCTAAATCAATTACCAGTGGCTGCTGCCAGT
GGTGCTTTTGCATGTCTTTCCGGGTTGGACTCAAGACGATAGTTACCGGATAAGGCGCAGCGGTCGGACT
GAACGGGGGGTTCGTGCATACAGTCCAGCTTGGAGCGAACTGCCTACCCGGAACTGAGTGTCAGGCGTGG
AATGAGACAAACGCGGCCATAACAGCGGAATGACACCGGTAAACCGAAAGGCAGGAACAGGAGAGCGCAC
GAGGGAGCCGCCAGGGGGAAACGCCTGGTATCTTTATAGTCCTGTCGGGTTTCGCCACCACTGATTTGAG
CGTCAGATTTCGTGATGCTTGTCAGGGGGGCGGAGCCTATGGAAAAACGGCTTTGCCGCGGCCCTCTCAC
TTCCCTGTTAAGTATCTTCCTGGCATCTTCCAGGAAATCTCCGCCCCGTTCGTAAGCCATTTCCGCTCGC
CGCAGTCGAACGACCGAGCGTAGCGAGTCAGTGAGCGAGGAAGCGGAATATATCCTGTATCACATATTCT
GCTGACGCACCGGTGCAGCCTTTTTTCTCCTGCCACATGAAGCACTTCACTGACACCCTCATCAGTGCCA
ACATAGTAAGCCAGTATACACTCCGCTAGCGCGATCGCTTAATTAATTTAAATCCTGCAGGGTTTAAACG
GCCGGCCTAGGGATAACAGGGTAATCGTAACTATAACGGTCCTAAGGTAGCGAATGATGTCCGGCGGTGC
TTTTGCCGTTACGCACCACCCCGTCAGTAGCTGAACAGGAGGGACAGCTGATAGAAACAGAAGCCACTGG
AGCACCTCAAAAACACCATCATACACTAAATCAGTAAGTTGGCAGCATCACCCGACGCACTTTGCGCCGA
ATAAATACCTGTGACGGAAGATCACTTCGCAGAATAAATAAATCCTGGTGTCCCTGTTGATACCGGGAAG
CCCTGGGCCAACTTTTGGCGAAAATGAGACGTTGTCGGCACGTAAGAGGTTCCAACTTTCACCATAATGA
AATAAGATCACTACCGGGCGTATTTTTTGAGTTATCGAGATTTTCAGGAGCTAAGGAAGCTAAAATGGAG
AAAAAAATCACTGGATATACCACCGTTGATATATCCCAATGGCATCGTAAAGAACATTTTGAGGCATTTC
AGTCAGTTGCTCAATGTACCTATAACCAGACCGTTCAGCTGGATATTACGGCCTTTTTAAAGACCGTAAA
GAAAAATAAGCACAAGTTTTATCCGGCCTTTATTCACATTCTTGCCCGCCTGATGAATGCTCATCCGGAA
TTCCGTATGGCAATGAAAGACGGTGAGCTGGTGATATGGGATAGTGTTCACCCTTGTTACACCGTTTTCC
ATGAGCAAACTGAAACGTTTTCATCGCTCTGGAGTGAATACCACGACGATTTCCGGCAGTTTCTACACAT
ATATTCGCAAGATGTGGCGTGTTACGGTGAAAACCTGGCCTATTTCCCTAAAGGGTTTATTGAGAATATG
TTTTTCGTCTCAGCCAATCCCTGGGTGAGTTTCACCAGTTTTGATTTAAACGTGGCCAATATGGACAACT
TCTTCGCCCCCGTTTTCACCATGGGCAAATATTATACGCAAGGCGACAAGGTGCTGATGCCGCTGGCGAT
TCAGGTTCATCATGCCGTCTGTGATGGCTTCCATGTCGGCAGAATGCTTAATGAATTACAACAGTACTGC
GATGAGTGGCAGGGCGGGGCGTAACCTGCAGGTTAATTAAGGAAGGGCGAATTCTGCAGATATCCATCAC
ACTGGCGGCCGCTCGAGCATGCATCTAGAGGGCCCAATTCGCCCTATAGTGAGTCGTATTACAATTCACT
GGCC (SEQ ID NO: 174)
The nucleotide sequence of PS1:
CATCATCAATAATATACCTTATAGATGGAATGGTGCCAATATGTAAATGAGGTGATTTTAAAAAGTGTGG
GCCGTGTGGTGATTGGCTGTGGGGTTAACGGTTAAAAGGGGCGGCGCGGCCGTGGGAAAATGACGTTTTA
TGGGGGTGGAGTTTTTTTGCAAGTTGTCGCGGGAAATGATAACTTCGTATAGCATACATTATACGAAGTT
ATTTACGCATAAAAAGGCTTCTTTTCTCACGGAACTACTTAGTTTTCCCACGGTATTTAACAGGAAATGA
GGTAGTTTTGACCGGATGCAAGTGAAAATTGCTGATTTTCGCGCGAAAACTGAATGAGGAAGTGTTTTTC
TGAATAATGTGGTATTTATGGCAGGGTGATAACTTCGTATAGCATACATTATACGAAGTTATGGAATGTT
TATGCCTTACCAGTGTAACATGAATCATGTGAAAGTGTTGTTGGAACCAGATGCCTTTTCCAGAATGAGC
CTAACAGGAATCTTTGACATGAACACGCAAATCTGGAAGATCCTGAGGTATGATGATACGAGATCGAGGG
TGCGCGCATGCGAATGCGGAGGCAAGCATGCCAGGTTCCAGCCGGTGTGTGTAGATGTGACCGAAGATCT
CAGACCGGATCATTTGGTTATTGCCCGCACTGGAGCAGAGTTCGGATCCAGTGGAGAAGAAACTGACTAA
GGTGAGTATTGGGAAAACTTTGGGGTGGGATTTTCAGATGGACAGATTGAGTAAAAATTTGTTTTTTCTG
TCTTGCAGCTGACATGAGTGGAAATGCTTCTTTTAAGGGGGGAGTCTTCAGCCCTTATCTGACAGGGCGT
CTCCCATCCTGGGCAGGAGTTCGTCAGAATGTTATGGGATCTACTGTGGATGGAAGACCCGTTCAACCCG
CCAATTCTTCAACGCTGACCTATGCTACTTTAAGTTCTTCACCTTTGGACGCAGCTGCAGCCGCTGCCGC
CGCCTCTGTCGCCGCTAACACTGTGCTTGGAATGGGTTACTATGGAAGCATCGTGGCTAATTCCACTTCC
TCTAATAACCCTTCTACACTGACTCAGGACAAGTTACTTGTCCTTTTGGCCCAGCTGGAGGCTTTGACCC
AACGTCTGGGTGAACTTTCTCAGCAGGTGGCCGAGTTGCGAGTACAAACTGAGTCTGCTGTCGGCACGGC
AAAGTCTAAATAAAAAAAATTCCAGAATCAATGAATAAATAAACGAGCTTGTTGTTGATTTAAAATCAAG
TGTTTTTATTTCATTTTTCGCGCACGGTATGCCCTGGACCACCGATCTCGATCATTGAGAACTCGGTGGA
TTTTTTCCAGAATCCTATAGAGGTGGGATTGAATGTTTAGATACATGGGCATTAGGCCGTCTTTGGGGTG
GAGATAGCTCCATTGAAGGGATTCATGCTCCGGGGTAGTGTTGTAAATCACCCAGTCATAACAAGGTCGC
AGTGCATGGTGTTGCACAATATCTTTTAGAAGTAGGCTGATTGCCACAGATAAGCCCTTGGTGTAGGTGT
TTACAAACCGGTTGAGCTGGGAGGGGTGCATTCGAGGTGAAATTATGTGCATTTTGGATTGGATTTTTAA
GTTGGCAATATTGCCGCCAAGATCCCGTCTTGGGTTCATGTTATGAAGGACTACCAAGACGGTGTATCCG
GTACATTTAGGAAATTTATCGTGCAGCTTGGATGGAAAAGCGTGGAAAAATTTGGAGACACCCTTGTGTC
CTCCGAGATTTTCCATGCACTCATCCATGATAATAGCAATGGGGCCGTGGGCAGCGGCGCGGGCAAACAC
GTTCCGTGGGTCTGACACATCATAGTTATGTTCCTGAGTTAAATCATCATAAGCCATTTTAATGAATTTG
GGGCGGAGCGTACCAGATTGGGGTATGAATGTTCCTTCGGGCCCCGGAGCATAGTTCCCCTCACAGATTT
GCATTTCCCAAGCTTTCAGTTCTGAGGGTGGAATCATGTCCACCTGGGGGGCTATGAAGAACACCGTTTC
GGGGGCGGGGGTGATTAGTTGGGATGATAGCAAGTTTCTGAGCAATTGAGATTTGCCACATCCGGTGGGG
CCATAAATAATTCCGATTACAGGTTGCAGGTGGTAGTTTAGGGAACGGCAACTGCCGTCTTCTCGAAGCA
AGGGGGCCACCTCGTTCATCATTTCCCTTACATGCATATTTTCCCGCACCAAATCCATTAGGAGGCGCTC
TCCTCCTAGTGATAGAAGTTCTTGTAGTGAGGAAAAGTTTTTCAGCGGTTTTAGACCGTCAGCCATGGGC
ATTTTGGAAAGAGTTTGCTGCAAAAGTTCTAGTCTGTTCCACAGTTCAGTGATGTGTTCTATGGCATCTC
GATCCAGCAGACCTCCTCGTTTCGCGGGTTTGGACGGCTCCTGGAGTAGGGTATGAGACGATGGGCGTCC
AGCGCTGCCAGGGTTCGGTCCTTCCAGGGTCTCAGTGTTCGAGTCAGGGTTGTTTCCGTCACAGTGAAGG
GGTGTGCGCCTGCTTGGGCGCTTGCCAGGGTGCGCTTCAGACTCATTCTGCTGGTGGAGAACTTCTGTCG
CTTGGCGCCCTGTATGTCGGCCAAGTAGCAGTTTACCATGAGTTCGTAGTTGAGCGCCTCGGCTGCGTGG
CCTTTGGCGCGGAGCTTACCTTTGGAAGTTTTCTTGCATACCGGGCAGTATAGGCATTTCAGCGCATACA
GCTTGGGCGCAAGGAAAATGGATTCTGGGGAGTATGCATCCGCGCCGCAGGAGGCGCAAACAGTTTCACA
TTCCACCAGCCAGGTTAAATCCGGTTCATTGGGGTCAAAAACAAGTTTTCCGCCATATTTTTTGATGCGT
TTCTTACCTTTGGTCTCCATAAGTTCGTGTCCTCGTTGAGTGACAAACAGGCTGTCCGTATCTCCGTAGA
CTGATTTTACAGGCCTCTTCTCCAGTGGAGTGCCTCGGTCTTCTTCGTACAGGAACTCTGACCACTCTGA
TACAAAGGCGCGCGTCCAGGCCAGCACAAAGGAGGCTATGTGGGAGGGGTAGCGATCGTTGTCAACCAGG
GGGTCCACCTTTTCCAAAGTATGCAAACACATGTCACCCTCTTCAACATCCAGGAATGTGATTGGCTTGT
AGGTGTATTTCACGTGACCTGGGGTCCCCGCTGGGGGGGTATAAAAGGGGGCGGTTCTTTGCTCTTCCTC
ACTGTCTTCCGGATCGCTGTCCAGGAACGTCAGCTGTTGGGGTAGGTATTCCCTCTCGAAGGCGGGCATG
ACCTCTGCACTCAGGTTGTCAGTTTCTAAGAACGAGGAGGATTTGATATTGACAGTGCCGGTTGAGATGC
CTTTCATGAGGTTTTCGTCCATTTGGTCAGAAAACACAATTTTTTTATTGTCAAGTTTGGTGGCAAATGA
TCCATACAGGGCGTTGGATAAAAGTTTGGCAATGGATCGCATGGTTTGGTTCTTTTCCTTGTCCGCGCGC
TCTTTGGCGGCGATGTTGAGTTGGACATACTCGCGTGCCAGGCACTTCCATTCGGGGAAGATAGTTGTTA
ATTCATCTGGCACGATTCTCACTTGCCACCCTCGATTATGCAAGGTAATTAAATCCACACTGGTGGCCAC
CTCGCCTCGAAGGGGTTCATTGGTCCAACAGAGCCTACCTCCTTTCCTAGAACAGAAAGGGGGAAGTGGG
TCTAGCATAAGTTCATCGGGAGGGTCTGCATCCATGGTAAAGATTCCCGGAAGTAAATCCTTATCAAAAT
AGCTGATGGGAGTGGGGTCATCTAAGGCCATTTGCCATTCTCGAGCTGCCAGTGCGCGCTCATATGGGTT
AAGGGGACTGCCCCAGGGCATGGGATGGGTGAGAGCAGAGGCATACATGCCACAGATGTCATAGACGTAG
ATGGGATCCTCAAAGATGCCTATGTAGGTTGGATAGCATCGCCCCCCTCTGATACTTGCTCGCACATAGT
CATATAGTTCATGTGATGGCGCTAGCAGCCCCGGACCCAAGTTGGTGCGATTGGGTTTTTCTGTTCTGTA
GACGATCTGGCGAAAGATGGCGTGAGAATTGGAAGAGATGGTGGGTCTTTGAAAAATGTTGAAATGGGCA
TGAGGTAGACCTACAGAGTCTCTGACAAAGTGGGCATAAGATTCTTGAAGCTTGGTTACCAGTTCGGCGG
TGACAAGTACGTCTAGGGCGCAGTAGTCAAGTGTTTCTTGAATGATGTCATAACCTGGTTGGTTTTTCTT
TTCCCACAGTTCGCGGTTGAGAAGGTATTCTTCGCGATCCTTCCAGTACTCTTCTAGCGGAAACCCGTCT
TTGTCTGCACGGTAAGATCCTAGCATGTAGAACTGATTAACTGCCTTGTAAGGGCAGCAGCCCTTCTCTA
CGGGTAGAGAGTATGCTTGAGCAGCTTTTCGTAGCGAAGCGTGAGTAAGGGCAAAGGTGTCTCTGACCAT
GACTTTGAGAAATTGGTATTTGAAGTCCATGTCGTCACAGGCTCCCTGTTCCCAGAGTTGGAAGTCTACC
CGTTTCTTGTAGGCGGGGTTGGGCAAAGCGAAAGTAACATCATTGAAGAGAATCTTACCGGCTCTGGGCA
TAAAATTGCGAGTGATGCGGAAAGGCTGTGGTACTTCCGCTCGATTGTTGATCACCTGGGCAGCTAGGAC
GATTTCGTCGAAACCGTTGATGTTGTGTCCTACGATGTATAATTCTATGAAACGCGGCGTGCCTCTGACG
TGAGGTAGCTTACTGAGCTCATCAAAGGTTAGGTCTGTGGGGTCAGATAAGGCGTAGTGTTCGAGAGCCC
ATTCGTGCAGGTGAGGATTTGCATGTAGGAATGATGACCAAAGATCTACCGCCAGTGCTGTTTGTAACTG
GTCCCGATACTGACGAAAATGCCGGCCAATTGCCATTTTTTCTGGAGTGACACAGTAGAAGGTTCTGGGG
TCTTGTTGCCATCGATCCCACTTGAGTTTAATGGCTAGATCGTGGGCCATGTTGACGAGACGCTCTTCTC
CTGAGAGTTTCATGACCAGCATGAAAGGAACTAGTTGTTTGCCAAAGGATCCCATCCAGGTGTAAGTTTC
CACATCGTAGGTCAGGAAGAGTCTTTCTGTGCGAGGATGAGAGCCGATCGGGAAGAACTGGATTTCCTGC
CACCAGTTGGAGGATTGGCTGTTGATGTGATGGAAGTAGAAGTTTCTGCGGCGCGCCGAGCATTCGTGTT
TGTGCTTGTACAGACGGCCGCAGTAGTCGCAGCGTTGCACGGGTTGTATCTCGTGAATGAGCTGTACCTG
GCTTCCCTTGACGAGAAATTTCAGTGGGAAGCCGAGGCCTGGCGATTGTATCTCGTGCTCTTCTATATTC
GCTGTATCGGCCTGTTCATCTTCTGTTTCGATGGTGGTCATGCTGACGAGCCCCCGCGGGAGGCAAGTCC
AGACCTCGGCGCGGGAGGGGCGGAGCTGAAGGACGAGAGCGCGCAGGCTGGAGCTGTCCAGAGTCCTGAG
ACGCTGCGGACTCAGGTTAGTAGGTAGGGACAGAAGATTAACTTGCATGATCTTTTCCAGGGCGTGCGGG
AGGTTCAGATGGTACTTGATTTCCACAGGTTCGTTTGTAGAGACGTCAATGGCTTGCAGGGTTCCGTGTC
CTTTGGGCGCCACTACCGTACCTTTGTTTTTTCTTTTGATCGGTGGTGGCTCTCTTGCTTCTTGCATGCT
CAGAAGCGGTGACGGGGACGCGCGCCGGGCGGCAGCGGTTGTTCCGGACCCGGGGGCATGGCTGGTAGTG
GCACGTCGGCGCCGCGCACGGGCAGGTTCTGGTATTGCGCTCTGAGAAGACTTGCGTGCGCCACCACGCG
TCGATTGACGTCTTGTATCTGACGTCTCTGGGTGAAAGCTACCGGCCCCGTGAGCTTGAACCTGAAAGAG
AGTTCAACAGAATCAATTTCGGTATCGTTAACGGCAGCTTGTCTCAGTATTTCTTGTACGTCACCAGAGT
TGTCCTGGTAGGCGATCTCCGCCATGAACTGCTCGATTTCTTCCTCCTGAAGATCTCCGCGACCCGCTCT
TTCGACGGTGGCCGCGAGGTCATTGGAGATACGGCCCATGAGTTGGGAGAATGCATTCATGCCCGCCTCG
TTCCAGACGCGGCTGTAAACCACGGCCCCCTCGGAGTCTCTTGCGCGCATCACCACCTGAGCGAGGTTAA
GCTCCACGTGTCTGGTGAAGACCGCATAGTTGCATAGGCGCTGAAAAAGGTAGTTGAGTGTGGTGGCAAT
GTGTTCGGCGACGAAGAAATACATGATCCATCGTCTCAGCGGCATTTCGCTAACATCGCCCAGAGCTTCC
AAGCGCTCCATGGCCTCGTAGAAGTCCACGGCAAAATTAAAAAACTGGGAGTTTCGCGCGGACACGGTCA
ATTCCTCCTCGAGAAGACGGATGAGTTCGGCTATGGTGGCCCGTACTTCGCGTTCGAAGGCTCCCGGGAT
CTCTTCTTCCTCTTCTATCTCTTCTTCCACTAACATCTCTTCTTCGTCTTCAGGCGGGGGCGGAGGGGGC
ACGCGGCGACGTCGACGGCGCACGGGCAAACGGTCGATGAATCGTTCAATGACCTCTCCGCGGCGGCGGC
GCATGGTTTCAGTGACGGCGCGGCCGTTCTCGCGCGGTCGCAGAGTAAAAACACCGCCGCGCATCTCCTT
AAAGTGGTGACTGGGAGGTTCTCCGTTTGGGAGGGAGAGGGCGCTGATTATACATTTTATTAATTGGCCC
GTAGGGACTGCGCGCAGAGATCTGATCGTGTCAAGATCCACGGGATCTGAAAACCTTTCGACGAAAGCGT
CTAACCAGTCACAGTCACAAGGTAGGCTGAGTACGGCTTCTTGTGGGCGGGGGTGGTTATGTGTTCGGTC
TGGGTCTTCTGTTTCTTCTTCATCTCGGGAAGGTGAGACGATGCTGCTGGTGATGAAATTAAAGTAGGCA
GTTCTAAGACGGCGGATGGTGGCGAGGAGCACCAGGTCTTTGGGTCCGGCTTGCTGGATACGCAGGCGAT
TGGCCATTCCCCAAGCATTATCCTGACATCTAGCAAGATCTTTGTAGTAGTCTTGCATGAGCCGTTCTAC
GGGCACTTCTTCCTCACCCGTTCTGCCATGCATACGTGTGAGTCCAAATCCGCGCATTGGTTGTACCAGT
GCCAAGTCAGCTACGACTCTTTCGGCGAGGATGGCTTGCTGTACTTGGGTAAGGGTGGCTTGAAAGTCAT
CAAAATCCACAAAGCGGTGGTAAGCCCCTGTATTAATGGTGTAAGCACAGTTGGCCATGACTGACCAGTT
AACTGTCTGGTGACCAGGGCGCACGAGCTCGGTGTATTTAAGGCGCGAATAGGCGCGGGTGTCAAAGATG
TAATCGTTGCAGGTGCGCACCAGATACTGGTACCCTATAAGAAAATGCGGCGGTGGTTGGCGGTAGAGAG
GCCATCGTTCTGTAGCTGGAGCGCCAGGGGCGAGGTCTTCCAACATAAGGCGGTGATAGCCGTAGATGTA
CCTGGACATCCAGGTGATTCCTGCGGCGGTAGTAGAAGCCCGAGGAAACTCGCGTACGCGGTTCCAAATG
TTGCGTAGCGGCATGAAGTAGTTCATTGTAGGCACGGTTTGACCAGTGAGGCGCGCGCAGTCATTGATGC
TCTATAGACACGGAGAAAATGAAAGCGTTCAGCGACTCGACTCCGTAGCCTGGAGGAACGTGAACGGGTT
GGGTCGCGGTGTACCCCGGTTCGAGACTTGTACTCGAGCCGGCCGGAGCCGCGGCTAACGTGGTATTGGC
ACTCCCGTCTCGACCCAGCCTACAAAAATCCAGGATACGGAATCGAGTCGTTTTGCTGGTTTCCGAATGG
CAGGGAAGTGAGTCCTATTTTTTTTTTTTTTGCCGCTCAGATGCATCCCGTGCTGCGACAGATGCGCCCC
CAACAACAGCCCCCCTCGCAGCAGCAGCAGCAGCAACCACAAAAGGCTGTCCCTGCAACTACTGCAACTG
CCGCCGTGAGCGGTGCGGGACAGCCCGCCTATGATCTGGACTTGGAAGAGGGCGAAGGACTGGCACGTCT
AGGTGCGCCTTCGCCCGAGCGGCATCCGCGAGTTCAACTGAAAAAAGATTCTCGCGAGGCGTATGTGCCC
CAACAGAACCTATTTAGAGACAGAAGCGGCGAGGAGCCGGAGGAGATGCGAGCTTCCCGCTTTAACGCGG
GTCGTGAGCTGCGTCACGGTTTGGACCGAAGACGAGTGTTGCGAGACGAGGATTTCGAAGTTGATGAAGT
GACAGGGATCAGTCCTGCCAGGGCACACGTGGCTGCAGCCAACCTTGTATCGGCTTACGAGCAGACAGTA
AAGGAAGAGCGTAACTTCCAAAAGTCTTTTAATAATCATGTGCGAACCCTGATTGCCCGCGAAGAAGTTA
CCCTTGGTTTGATGCATTTGTGGGATTTGATGGAAGCTATCATTCAGAACCCTACTAGCAAACCTCTGAC
CGCCCAGCTGTTTCTGGTGGTGCAACACAGCAGAGACAATGAGGCTTTCAGAGAGGCGCTGCTGAACATC
ACCGAACCCGAGGGGAGATGGTTGTATGATCTTATCAACATTCTACAGAGTATCATAGTGCAGGAGCGGA
GCCTGGGCCTGGCCGAGAAGGTAGCTGCCATCAATTACTCGGTTTTGAGCTTGGGAAAATATTACGCTCG
CAAAATCTACAAGACTCCATACGTTCCCATAGACAAGGAGGTGAAGATAGATGGGTTCTACATGCGCATG
ACGCTCAAGGTCTTGACCCTGAGCGATGATCTTGGGGTGTATCGCAATGACAGAATGCATCGCGCGGTTA
GCGCCAGCAGGAGGCGCGAGTTAAGCGACAGGGAACTGATGCACAGTTTGCAAAGAGCTCTGACTGGAGC
TGGAACCGAGGGTGAGAATTACTTCGACATGGGAGCTGACTTGCAGTGGCAGCCTAGTCGCAGGGCTCTG
AGCGCCGCGACGGCAGGATGTGAGCTTCCTTACATAGAAGAGGCGGATGAAGGCGAGGAGGAAGAGGGCG
AGTACTTGGAAGACTGATGGCACAACCCGTGTTTTTTGCTAGATGGAACAGCAAGCACCGGATCCCGCAA
TGCGGGCGGCGCTGCAGAGCCAGCCGTCCGGCATTAACTCCTCGGACGATTGGACCCAGGCCATGCAACG
TATCATGGCGTTGACGACTCGCAACCCCGAAGCCTTTAGACAGCAACCCCAGGCCAACCGTCTATCGGCC
ATCATGGAAGCTGTAGTGCCTTCCCGATCTAATCCCACTCATGAGAAGGTCCTGGCCATCGTGAACGCGT
TGGTGGAGAACAAAGCTATTCGTCCAGATGAGGCCGGACTGGTATACAACGCTCTCTTAGAACGCGTGGC
TCGCTACAACAGTAGCAATGTGCAAACCAATTTGGACCGTATGATAACAGATGTACGCGAAGCCGTGTCT
CAGCGCGAAAGGTTCCAGCGTGATGCCAACCTGGGTTCGCTGGTGGCGTTAAATGCTTTCTTGAGTACTC
AGCCTGCTAATGTGCCGCGTGGTCAACAGGATTATACTAACTTTTTAAGTGCTTTGAGACTGATGGTATC
AGAAGTACCTCAGAGCGAAGTGTATCAGTCCGGTCCTGATTACTTCTTTCAGACTAGCAGACAGGGCTTG
CAGACGGTAAATCTGAGCCAAGCTTTTAAAAACCTTAAAGGTTTGTGGGGAGTGCATGCCCCGGTAGGAG
AAAGAGCAACCGTGTCTAGCTTGTTAACTCCGAACTCCCGCCTGTTATTACTGTTGGTAGCTCCTTTCAC
CGACAGCGGTAGCATCGACCGTAATTCCTATTTGGGTTACCTACTAAACCTGTATCGCGAAGCCATAGGG
CAAAGTCAGGTGGACGAGCAGACCTATCAAGAAATTACCCAAGTCAGTCGCGCTTTGGGACAGGAAGACA
CTGGCAGTTTGGAAGCCACTCTGAACTTCTTGCTTACCAATCGGTCTCAAAAGATCCCTCCTCAATATGC
TCTTACTGCGGAGGAGGAGAGGATCCTTAGATATGTGCAGCAGAGCGTGGGATTGTTTCTGATGCAAGAG
GGGGCAACTCCGACTGCAGCACTGGACATGACAGCGCGAAATATGGAGCCCAGCATGTATGCCAGTAACC
GACCTTTCATTAACAAACTGCTGGACTACTTGCACAGAGCTGCCGCTATGAACTCTGATTATTTCACCAA
TGCCATCTTAAACCCGCACTGGCTGCCCCCACCTGGTTTCTACACGGGCGAATATGACATGCCCGACCCT
AATGACGGATTTCTGTGGGACGACGTGGACAGCGATGTTTTTTCACCTCTTTCTGATCATCGCACGTGGA
AAAAGGAAGGCGGTGATAGAATGCATTCTTCTGCATCGCTGTCCGGGGTCATGGGTGCTACCGCGGCTGA
GCCCGAGTCTGCAAGTCCTTTTCCTAGTCTACCCTTTTCTCTACACAGTGTACGTAGCAGCGAAGTGGGT
AGAATAAGTCGCCCGAGTTTAATGGGCGAAGAGGAGTACCTAAACGATTCCTTGCTCAGACCGGCAAGAG
AAAAAAATTTCCCAAACAATGGAATAGAAAGTTTGGTGGATAAAATGAGTAGATGGAAGACTTATGCTCA
GGATCACAGAGACGAGCCTGGGATCATGGGGACTACAAGTAGAGCGAGCCGTAGACGCCAGCGCCATGAC
AGACAGAGGGGTCTTGTGTGGGACGATGAGGATTCGGCCGATGATAGCAGCGTGTTGGACTTGGGTGGGA
GAGGAAGGGGCAACCCGTTTGCTCATTTGCGCCCTCGCTTGGGTGGTATGTTGTGAAAAAAAATAAAAAA
GAAAAACTCACCAAGGCCATGGCGACGAGCGTACGTTCGTTCTTCTTTATTATCTGTGTCTAGTATAATG
AGGCGAGTCGTGCTAGGCGGAGCGGTGGTGTATCCGGAGGGTCCTCCTCCTTCGTACGAGAGCGTGATGC
AGCAGCAGCAGGCGACGGCGGTGATGCAATCCCCACTGGAGGCTCCCTTTGTGCCTCCGCGATACCTGGC
ACCTACGGAGGGCAGAAACAGCATTCGTTACTCGGAACTGGCACCTCAGTACGATACCACCAGGTTGTAT
CTGGTGGACAACAAGTCGGCGGACATTGCTTCTCTGAACTATCAGAATGACCACAGCAACTTCTTGACCA
CGGTGGTGCAGAACAATGACTTTACCCCTACGGAAGCCAGCACCCAGACCATTAACTTTGATGAACGATC
GCGGTGGGGCGGTCAGCTAAAGACCATCATGCATACTAACATGCCAAACGTGAACGAGTATATGTTTAGT
AACAAGTTCAAAGCGCGTGTGATGGTGTCCAGAAAACCTCCCGACGGTGCTGCAGTTGGGGATACTTATG
ATCACAAGCAGGATATTTTGGAATATGAGTGGTTCGAGTTTACTTTGCCAGAAGGCAACTTTTCAGTTAC
TATGACTATTGATTTGATGAACAATGCCATCATAGATAATTACTTGAAAGTGGGTAGACAGAATGGAGTG
CTTGAAAGTGACATTGGTGTTAAGTTCGACACCAGGAACTTCAAGCTGGGATGGGATCCCGAAACCAAGT
TGATCATGCCTGGAGTGTATACGTATGAAGCCTTCCATCCTGACATTGTCTTACTGCCTGGCTGCGGAGT
GGATTTTACCGAGAGTCGTTTGAGCAACCTTCTTGGTATCAGAAAAAAACAGCCATTTCAAGAGGGTTTT
AAGATTTTGTATGAAGATTTAGAAGGTGGTAATATTCCGGCCCTCTTGGATGTAGATGCCTATGAGAACA
GTAAGAAAGAACAAAAAGCCAAAATAGAAGCTGCTACAGCTGCTGCAGAAGCTAAGGCAAACATAGTTGC
CAGCGACTCTACAAGGGTTGCTAACGCTGGAGAGGTCAGAGGAGACAATTTTGCGCCAACACCTGTTCCG
ACTGCAGAATCATTATTGGCCGATGTGTCTGAAGGAACGGACGTGAAACTCACTATTCAACCTGTAGAAA
AAGATAGTAAGAATAGAAGCTATAATGTGTTGGAAGACAAAATCAACACAGCCTATCGCAGTTGGTATCT
TTCGTACAATTATGGCGATCCCGAAAAAGGAGTGCGTTCCTGGACATTGCTCACCACCTCAGATGTCACC
TGCGGAGCAGAGCAGGTTTACTGGTCGCTTCCAGACATGATGAAGGATCCTGTCACTTTCCGCTCCACTA
GACAAGTCAGTAACTACCCTGTGGTGGGTGCAGAGCTTATGCCCGTCTTCTCAAAGAGCTTCTACAACGA
ACAAGCTGTGTACTCCCAGCAGCTCCGCCAGTCCACCTCGCTTACGCACGTCTTCAACCGCTTTCCTGAG
AACCAGATTTTAATCCGTCCGCCGGCGCCCACCATTACCACCGTCAGTGAAAACGTTCCTGCTCTCACAG
ATCACGGGACCCTGCCGTTGCGCAGCAGTATCCGGGGAGTCCAACGTGTGACCGTTACTGACGCCAGACG
CCGCACCTGTCCCTACGTGTACAAGGCACTGGGCATAGTCGCACCGCGCGTCCTTTCAAGCCGCACTTTC
TAAAAAAAAAATGTCCATTCTTATCTCGCCCAGTAATAACACCGGTTGGGGTCTGCGCGCTCCAAGCAAG
ATGTACGGAGGCGCACGCAAACGTTCTACCCAACATCCCGTGCGTGTTCGCGGACATTTTCGCGCTCCAT
GGGGTGCCCTCAAGGGCCGCACTCGCGTTCGAACCACCGTCGATGATGTAATCGATCAGGTGGTTGCCGA
CGCCCGTAATTATACTCCTACTGCGCCTACATCTACTGTGGATGCAGTTATTGACAGTGTAGTGGCTGAC
GCTCGCAACTATGCTCGACGTAAGAGCCGGCGAAGGCGCATTGCCAGACGCCACCGAGCTACCACTGCCA
TGCGAGCCGCAAGAGCTCTGCTACGAAGAGCTAGACGCGTGGGGCGAAGAGCCATGCTTAGGGCGGCCAG
ACGTGCAGCTTCGGGCGCCAGCGCCGGCAGGTCCCGCAGGCAAGCAGCCGCTGTCGCAGCGGCGACTATT
GCCGACATGGCCCAATCGCGAAGAGGCAATGTATACTGGGTGCGTGACGCTGCCACCGGTCAACGTGTAC
CCGTGCGCACCCGTCCCCCTCGCACTTAGAAGATACTGAGCAGTCTCCGATGTTGTGTCCCAGCGGCGAG
GATGTCCAAGCGCAAATACAAGGAAGAAATGCTGCAGGTTATCGCACCTGAAGTCTACGGCCAACCGTTG
AAGGATGAAAAAAAACCCCGCAAAATCAAGCGGGTTAAAAAGGACAAAAAAGAAGAGGAAGATGGCGATG
ATGGGCTGGCGGAGTTTGTGCGCGAGTTTGCCCCACGGCGACGCGTGCAATGGCGTGGGCGCAAAGTTCG
ACATGTGTTGAGACCTGGAACTTCGGTGGTCTTTACACCCGGCGAGCGTTCAAGCGCTACTTTTAAGCGT
TCCTATGATGAGGTGTACGGGGATGATGATATTCTTGAGCAGGCGGCTGACCGATTAGGCGAGTTTGCTT
ATGGCAAGCGTAGTAGAATAACTTCCAAGGATGAGACAGTGTCAATACCCTTGGATCATGGAAATCCCAC
CCCTAGTCTTAAACCGGTCACTTTGCAGCAAGTGTTACCCGTAACTCCGCGAACAGGTGTTAAACGCGAA
GGTGAAGATTTGTATCCCACTATGCAACTGATGGTACCCAAACGCCAGAAGTTGGAGGACGTTTTGGAGA
AAGTAAAAGTGGATCCAGATATTCAACCTGAGGTTAAAGTGAGACCCATTAAGCAGGTAGCGCCTGGTCT
GGGGGTACAAACTGTAGACATTAAGATTCCCACTGAAAGTATGGAAGTGCAAACTGAACCCGCAAAGCCT
ACTGCCACCTCCACTGAAGTGCAAACGGATCCATGGATGCCCATGCCTATTACAACTGACGCCGCCGGTC
CCACTCGAAGATCCCGACGAAAGTACGGTCCAGCAAGTCTGTTGATGCCCAATTATGTTGTACACCCATC
TATTATTCCTACTCCTGGTTACCGAGGCACTCGCTACTATCGCAGCCGAAACAGTACCTCCCGCCGTCGC
CGCAAGACACCTGCAAATCGCAGTCGTCGCCGTAGACGCACAAGCAAACCGACTCCCGGCGCCCTGGTGC
GGCAAGTGTACCGCAATGGTAGTGCGGAACCTTTGACACTGCCGCGTGCGCGTTACCATCCGAGTATCAT
CACTTAATCAATGTTGCCGCTGCCTCCTTGCAGATATGGCCCTCACTTGTCGCCTTCGCGTTCCCATCAC
TGGTTACCGAGGAAGAAACTCGCGCCGTAGAAGAGGGATGTTGGGACGCGGAATGCGACGCTACAGGCGA
CGGCGTGCTATCCGCAAGCAATTGCGGGGTGGTTTTTTACCAGCCTTAATTCCAATTATCGCTGCTGCAA
TTGGCGCGATACCAGGCATAGCTTCCGTGGCGGTTCAGGCCTCGCAACGACATTGACATTGGAAAAAAAA
CGTATAAATAAAAAAAAATACAATGGACTCTGACACTCCTGGTCCTGTGACTATGTTTTCTTAGAGATGG
AAGACATCAATTTTTCATCCTTGGCTCCGCGACACGGCACGAAGCCGTACATGGGCACCTGGAGCGACAT
CGGCACGAGCCAACTGAACGGGGGCGCCTTCAATTGGAGCAGTATCTGGAGCGGGCTTAAAAATTTTGGC
TCAACCATAAAAACATACGGGAACAAAGCTTGGAACAGCAGTACAGGACAGGCGCTTAGAAATAAACTTA
AAGACCAGAACTTCCAACAAAAAGTAGTCGATGGGATAGCTTCCGGCATCAATGGAGTGGTAGATTTGGC
TAACCAGGCTGTGCAGAAAAAGATAAACAGTCGTTTGGACCCGCCGCCAGCAACCCCAGGTGAAATGCAA
GTGGAGGAAGAAATTCCTCCGCCAGAAAAACGAGGCGACAAGCGTCCGCGTCCCGATTTGGAAGAGACGC
TGGTGACGCGCGTAGATGAACCGCCTTCTTATGAGGAAGCAACGAAGCTTGGAATGCCCACCACTAGACC
GATAGCCCCAATGGCCACCGGGGTGATGAAACCTTCTCAGTTGCATCGACCCGTCACCTTGGATTTGCCC
CCTCCCCCTGCTGCTACTGCTGTACCCGCTTCTAAGCCTGTCGCTGCCCCGAAACCAGTCGCCGTAGCCA
GGTCACGTCCCGGGGGCGCTCCTCGTCCAAATGCGCACTGGCAAAATACTCTGAACAGCATCGTGGGTCT
AGGCGTGCAAAGTGTAAAACGCCGTCGCTGCTTTTAATTAAATATGGAGTAGCGCTTAACTTGCCTATCT
GTGTATATGTGTCATTACACGCCGTCACAGCAGCAGAGGAAAAAAGGAAGAGGTCGTGCGTCGACGCTGA
GTTACTTTCAAGATGGCCACCCCATCGATGCTGCCCCAATGGGCATACATGCACATCGCCGGACAGGATG
CTTCGGAGTACCTGAGTCCGGGTCTGGTGCAGTTCGCCCGCGCCACAGACACCTACTTCAATCTGGGAAA
TAAGTTTAGAAATCCCACCGTAGCGCCGACCCACGATGTGACCACCGACCGTAGCCAGCGGCTCATGTTG
CGCTTCGTGCCCGTTGACCGGGAGGACAATACATACTCTTACAAAGTGCGGTACACCCTGGCCGTGGGCG
ACAACAGAGTGCTGGATATGGCCAGCACGTTCTTTGACATTAGGGGCGTGTTGGACAGAGGTCCCAGTTT
CAAACCCTATTCTGGTACGGCTTACAACTCTCTGGCTCCTAAAGGCGCTCCAAATGCATCTCAATGGATT
GCAAAAGGCGTACCAACTGCAGCAGCCGCAGGCAATGGTGAAGAAGAACATGAAACAGAGGAGAAAACTG
CTACTTACACTTTTGCCAATGCTCCTGTAAAAGCCGAGGCTCAAATTACAAAAGAGGGCTTACCAATAGG
TTTGGAGATTTCAGCTGAAAACGAATCTAAACCCATCTATGCAGATAAACTTTATCAGCCAGAACCTCAA
GTGGGAGATGAAACTTGGACTGACCTAGACGGAAAAACCGAAGAGTATGGAGGCAGGGCTCTAAAGCCTA
CTACTAACATGAAACCCTGTTACGGGTCCTATGCGAAGCCTACTAATTTAAAAGGTGGTCAGGCAAAACC
GAAAAACTCGGAACCGTCGAGTGAAAAAATTGAATATGATATTGACATGGAATTTTTTGATAACTCATCG
CAAAGAACAAACTTCAGTCCTAAAATTGTCATGTATGCAGAAAATGTAGGTTTGGAAACGCCAGACACTC
ATGTAGTGTACAAACCTGGAACAGAAGACACAAGTTCCGAAGCTAATTTGGGACAACAGTCTATGCCCAA
CAGACCCAACTACATTGGCTTCAGAGATAACTTTATTGGACTCATGTACTATAACAGTACTGGTAACATG
GGGGTGCTGGCTGGTCAAGCGTCTCAGTTAAATGCAGTGGTTGACTTGCAGGACAGAAACACAGAACTTT
CTTACCAACTCTTGCTTGACTCTCTGGGCGACAGAACCAGATACTTTAGCATGTGGAATCAGGCTGTGGA
CAGTTATGATCCTGATGTACGTGTTATTGAAAATCATGGTGTGGAAGATGAACTTCCCAACTATTGTTTT
CCACTGGACGGCATAGGTGTTCCAACAACCAGTTACAAATCAATAGTTCCAAATGGAGAAGATAATAATA
ATTGGAAAGAACCTGAAGTAAATGGAACAAGTGAGATCGGACAGGGTAATTTGTTTGCCATGGAAATTAA
CCTTCAAGCCAATCTATGGCGAAGTTTCCTTTATTCCAATGTGGCTCTGTATCTCCCAGACTCGTACAAA
TACACCCCGTCCAATGTCACTCTTCCAGAAAACAAAAACACCTACGACTACATGAACGGGCGGGTGGTGC
CGCCATCTCTAGTAGACACCTATGTGAACATTGGTGCCAGGTGGTCTCTGGATGCCATGGACAATGTCAA
CCCATTCAACCACCACCGTAACGCTGGCTTGCGTTACCGATCTATGCTTCTGGGTAACGGACGTTATGTG
CCTTTCCACATACAAGTGCCTCAAAAATTCTTCGCTGTTAAAAACCTGCTGCTTCTCCCAGGCTCCTACA
CTTATGAGTGGAACTTTAGGAAGGATGTGAACATGGTTCTACAGAGTTCCCTCGGTAACGACCTGCGGGT
AGATGGCGCCAGCATCAGTTTCACGAGCATCAACCTCTATGCTACTTTTTTCCCCATGGCTCACAACACC
GCTTCCACCCTTGAAGCCATGCTGCGGAATGACACCAATGATCAGTCATTCAACGACTACCTATCTGCAG
CTAACATGCTCTACCCCATTCCTGCCAATGCAACCAATATTCCCATTTCCATTCCTTCTCGCAACTGGGC
GGCTTTCAGAGGCTGGTCATTTACCAGACTGAAAACCAAAGAAACTCCCTCTTTGGGGTCTGGATTTGAC
CCCTACTTTGTCTATTCTGGTTCTATTCCCTACCTGGATGGTACCTTCTACCTGAACCACACTTTTAAGA
AGGTTTCCATCATGTTTGACTCTTCAGTGAGCTGGCCTGGAAATGACAGGTTACTATCTCCTAACGAATT
TGAAATAAAGCGCACTGTGGATGGCGAAGGCTACAACGTAGCCCAATGCAACATGACCAAAGACTGGTTC
TTGGTACAGATGCTCGCCAACTACAACATCGGCTATCAGGGCTTCTACATTCCAGAAGGATACAAAGATC
GCATGTATTCATTTTTCAGAAACTTCCAGCCCATGAGCAGGCAGGTGGTTGATGAGGTCAATTACAAAGA
CTTCAAGGCCGTCGCCATACCCTACCAACACAACAACTCTGGCTTTGTGGGTTACATGGCTCCGACCATG
CGCCAAGGTCAACCCTATCCCGCTAACTATCCCTATCCACTCATTGGAACAACTGCCGTAAATAGTGTTA
CGCAGAAAAAGTTCTTGTGTGACAGAACCATGTGGCGCATACCGTTCTCGAGCAACTTCATGTCTATGGG
GGCCCTTACAGACTTGGGACAGAATATGCTCTATGCCAACTCAGCTCATGCTCTGGACATGACCTTTGAG
GTGGATCCCATGGATGAGCCCACCCTGCTTTATCTTCTCTTCGAAGTTTTCGACGTGGTCAGAGTGCATC
AGCCACACCGCGGCATCATCGAGGCAGTCTACCTGCGTACACCGTTCTCGGCCGGTAACGCTACCACGTA
AGAAGCTTCTTGCTTCTTGCAAATAGCAGCTGCAACCATGGCCTGCGGATCCCAAAACGGCTCCAGCGAG
CAAGAGCTCAGAGCCATTGTCCAAGACCTGGGTTGCGGACCCTATTTTTTGGGAACCTACGATAAGCGCT
TCCCGGGGTTCATGGCCCCCGATAAGCTCGCCTGTGCCATTGTAAATACGGCCGGACGTGAGACGGGGGG
AGAGCACTGGTTGGCTTTCGGTTGGAACCCACGTTCTAACACCTGCTACCTTTTTGATCCTTTTGGATTC
TCGGATGATCGTCTCAAACAGATTTACCAGTTTGAATATGAGGGTCTCCTGCGCCGCAGCGCTCTTGCTA
CCAAGGACCGCTGTATTACGCTGGAAAAATCTACCCAGACCGTGCAGGGCCCCCGTTCTGCCGCCTGCGG
ACTTTTCTGCTGCATGTTCCTTCACGCCTTTGTGCACTGGCCTGACCGTCCCATGGACGGAAACCCCACC
ATGAAATTGCTAACTGGAGTGCCAAACAACATGCTTCATTCTCCTAAAGTCCAGCCCACCCTGTGTGACA
ATCAAAAAGCACTCTACCATTTTCTTAATACCCATTCGCCTTATTTTCGCTCTCATCGTACACACATCGA
AAGGGCCACTGCGTTCGACCGTATGGATGTTCAATAATGACTCATGTAAACAACGTGTTCAATAAACATC
ACTTTATTTTTTTACATGTATCAAGGCTCTGGATTACTTATTTATTTACAAGTCGAATGGGTTCTGACGA
GAATCAGAATGACCCGCAGGCAGTGATACGTTGCGGAACTGATACTTGGGTTGCCACTTGAATTCGGGAA
TCACCAACTTGGGAACCGGTATATCGGGCAGGATGTCACTCCACAGCTTTCTGGTCAGCTGCAAAGCTCC
AAGCAGGTCAGGAGCCGAAATCTTGAAATCACAATTAGGACCAGTGCTCTGAGCGCGAGAGTTGCGGTAC
ACCGGATTGCAGCACTGAAACACCATCAGCGACGGATGTCTCACGCTTGCCAGCACGGTGGGATCTGCAA
TCATGCCCACATCCAGATCTTCAGCATTGGCAATGCTGAACGGGGTCATCTTGCAGGTCTGCCTACCCAT
GGCGGGCACCCAATTAGGCTTGTGGTTGCAATCGCAGTGCAGGGGGATCAGTATCATCTTGGCCTGATCC
TGTCTGATTCCTGGATACACGGCTCTCATGAAAGCATCATATTGCTTGAAAGCCTGCTGGGCTTTACTAC
CCTCGGTATAAAACATCCCGCAGGACCTGCTCGAAAACTGGTTAGCTGCACAGCCGGCATCATTCACACA
GCAGCGGGCGTCATTGTTGGCTATTTGCACCACACTTCTGCCCCAGCGGTTTTGGGTGATTTTGGTTCGC
TCGGGATTCTCCTTTAAGGCTCGTTGTCCGTTCTCGCTGGCCACATCCATCTCGATAATCTGCTCCTTCT
GAATCATAATATTGCCATGCAGGCACTTCAGCTTGCCCTCATAATCATTGCAGCCATGAGGCCACAACGC
ACAGCCTGTACATTCCCAATTATGGTGGGCGATCTGAGAAAAAGAATGTATCATTCCCTGCAGAAATCTT
CCCATCATCGTGCTCAGTGTCTTGTGACTAGTGAAAGTTAACTGGATGCCTCGGTGCTCTTCGTTTACGT
ACTGGTGACAGATGCGCTTGTATTGTTCGTGTTGCTCAGGCATTAGTTTAAAACAGGTTCTAAGTTCGTT
ATCCAGCCTGTACTTCTCCATCAGCAGACACATCACTTCCATGCCTTTCTCCCAAGCAGACACCAGGGGC
AAGCTAATCGGATTCTTAACAGTGCAGGCAGCAGCTCCTTTAGCCAGAGGGTCATCTTTAGCGATCTTCT
CAATGCTTCTTTTGCCATCCTTCTCAACGATGCGCACGGGCGGGTAGCTGAAACCCACTGCTACAAGTTG
CGCCTCTTCTCTTTCTTCTTCGCTGTCTTGACTGATGTCTTGCATGGGGATATGTTTGGTCTTCCTTGGC
TTCTTTTTGGGGGGTATCGGAGGAGGAGGACTGTCGCTCCGTTCCGGAGACAGGGAGGATTGTGACGTTT
CGCTCACCATTACCAACTGACTGTCGGTAGAAGAACCTGACCCCACACGGCGACAGGTGTTTTTCTTCGG
GGGCAGAGGTGGAGGCGATTGCGAAGGGCTGCGGTCCGACCTGGAAGGCGGATGACTGGCAGAACCCCTT
CCGCGTTCGGGGGTGTGCTCCCTGTGGCGGTCGCTTAACTGATTTCCTTCGCGGCTGGCCATTGTGTTCT
CCTAGGCAGAGAAACAACAGACATGGAAACTCAGCCATTGCTGTCAACATCGCCACGAGTGCCATCACAT
CTCGTCCTCAGCGACGAGGAAAAGGAGCAGAGCTTAAGCATTCCACCGCCCAGTCCTGCCACCACCTCTA
CCCTAGAAGATAAGGAGGTCGACGCATCTCATGACATGCAGAATAAAAAAGCGAAAGAGTCTGAGACAGA
CATCGAGCAAGACCCGGGCTATGTGACACCGGTGGAACACGAGGAAGAGTTGAAACGCTTTCTAGAGAGA
GAGGATGAAAACTGCCCAAAACAGCGAGCAGATAACTATCACCAAGATGCTGGAAATAGGGATCAGAACA
CCGACTACCTCATAGGGCTTGACGGGGAAGACGCGCTCCTTAAACATCTAGCAAGACAGTCGCTCATAGT
CAAGGATGCATTATTGGACAGAACTGAAGTGCCCATCAGTGTGGAAGAGCTCAGCTGCGCCTACGAGCTT
AACCTTTTTTCACCTCGTACTCCCCCCAAACGTCAGCCAAACGGCACCTGCGAGCCAAATCCTCGCTTAA
ACTTTTATCCAGCTTTTGCTGTGCCAGAAGTACTGGCTACCTATCACATCTTTTTTAAAAATCAAAAAAT
TCCAGTCTCCTGCCGCGCTAATCGCACCCGCGCCGATGCCCTACTCAATCTGGGACCTGGTTCACGCTTA
CCTGATATAGCTTCCTTGGAAGAGGTTCCAAAGATCTTCGAGGGTCTGGGCAATAATGAGACTCGGGCCG
CAAATGCTCTGCAAAAGGGAGAAAATGGCATGGATGAGCATCACAGCGTTCTGGTGGAATTGGAAGGCGA
TAATGCCAGACTCGCAGTACTCAAGCGAAGCGTCGAGGTCACACACTTCGCATATCCCGCTGTCAACCTG
CCCCCTAAAGTCATGACGGCGGTCATGGACCAGTTACTCATTAAGCGCGCAAGTCCCCTTTCAGAAGACA
TGCATGACCCAGATGCCTGTGATGAGGGTAAACCAGTGGTCAGTGATGAGCAGCTAACCCGATGGCTGGG
CACCGACTCTCCCCGGGATTTGGAAGAGCGTCGCAAGCTTATGATGGCCGTGGTGCTGGTTACCGTAGAA
CTAGAGTGTCTCCGACGTTTCTTTACCGATTCAGAAACCTTGCGCAAACTCGAAGAGAATCTGCACTACA
CTTTTAGACACGGCTTTGTGCGGCAGGCATGCAAGATATCTAACGTGGAACTCACCAACCTGGTTTCCTA
CATGGGTATTCTGCATGAGAATCGCCTAGGACAAAGCGTGCTGCACAGCACCCTTAAGGGGGAAGCCCGC
CGTGATTACATCCGCGATTGTGTCTATCTCTACCTGTGCCACACGTGGCAAACCGGCATGGGTGTATGGC
AGCAATGTTTAGAAGAACAGAACTTGAAAGAGCTTGACAAGCTCTTACAGAAATCTCTTAAGGTTCTGTG
GACAGGGTTCGACGAGCGCACCGTCGCTTCCGACCTGGCAGACCTCATCTTCCCAGAGCGTCTCAGGGTT
ACTTTGCGAAACGGATTGCCTGACTTTATGAGCCAGAGCATGCTTAACAATTTTCGCTCTTTCATCCTGG
AACGCTCCGGTATCCTGCCCGCCACCTGCTGCGCACTGCCCTCCGACTTTGTGCCTCTCACCTACCGCGA
GTGCCCCCCGCCGCTATGGAGTCACTGCTACCTGTTCCGTCTGGCCAACTATCTCTCCTACCACTCGGAT
GTGATCGAGGATGTGAGCGGAGACGGCTTGCTGGAGTGCCACTGCCGCTGCAATCTGTGCACGCCCCACC
GGTCCCTAGCTTGCAACCCCCAGTTGATGAGCGAAACCCAGATAATAGGCACCTTTGAATTGCAAGGCCC
CAGCAGCCAAGGCGATGGGTCTTCTCCTGGGCAAAGTTTAAAACTGACCCCGGGACTGTGGACCTCCGCC
TACTTGCGCAAGTTTGCTCCGGAAGATTACCACCCCTATGAAATCAAGTTCTATGAGGACCAATCACAGC
CTCCAAAGGCCGAACTTTCGGCTTGCGTCATCACCCAGGGGGCAATTCTGGCCCAATTGCAAGCCATCCA
AAAATCCCGCCAAGAATTTCTACTGAAAAAGGGTAAGGGGGTCTACCTTGACCCCCAGACCGGCGAGGAA
CTCAACACAAGGTTCCCTCAGGATGTCCCAACGACGAGAAAACAAGAAGTTGAAGGTGCAGCCGCCGCCC
CCAGAAGATATGGAGGAAGATTGGGACAGTCAGGCAGAGGAGGCGGAGGAGGACAGTCTGGAGGACAGTC
TGGAGGAAGACAGTTTGGAGGAGGAAAACGAGGAGGCAGAGGAGGTGGAAGAAGTAACCGCCGACAAACA
GTTATCCTCGGCTGCGGAGACAAGCAACAGCGCTACCATCTCCGCTCCGAGTCGAGGAACCCGGCGGCGT
CCCAGCAGTAGATGGGACGAGACCGGACGCTTCCCGAACCCAACCAGCGCTTCCAAGACCGGTAAGAAGG
ATCGGCAGGGATACAAGTCCTGGCGGGGGCATAAGAATGCCATCATCTCCTGCTTGCATGAGTGCGGGGG
CAACATATCCTTCACGCGGCGCTACTTGCTATTCCACCATGGGGTGAACTTTCCGCGCAATGTTTTGCAT
TACTACCGTCACCTCCACAGCCCCTACTATAGCCAGCAAATCCCGACAGTCTCGACAGATAAAGACAGCG
GCGGCGACCTCCAACAGAAAACCAGCAGCGGCAGTTAGAAAATACACAACAAGTGCAGCAACAGGAGGAT
TAAAGATTACAGCCAACGAGCCAGCGCAAACCCGAGAGTTAAGAAATCGGATCTTTCCAACCCTGTATGC
CATCTTCCAGCAGAGTCGGGGTCAAGAGCAGGAACTGAAAATAAAAAACCGATCTCTGCGTTCGCTCACC
AGAAGTTGTTTGTATCACAAGAGCGAAGATCAACTTCAGCGCACTCTCGAGGACGCCGAGGCTCTCTTCA
ACAAGTACTGCGCGCTGACTCTTAAAGAGTAGGCAGCGACCGCGCTTATTCAAAAAAGGCGGGAATTACA
TCATCCTCGACATGAGTAAAGAAATTCCCACGCCTTACATGTGGAGTTATCAACCCCAAATGGGATTGGC
AGCAGGCGCCTCCCAGGACTACTCCACCCGCATGAATTGGCTCAGCGCCGGGCCTTCTATGATTTCTCGA
GTTAATGATATACGCGCCTACCGAAACCAAATACTTTTGGAACAGTCAGCTCTTACCACCACGCCCCGCC
AACACCTTAATCCCAGAAATTGGCCCGCCGCCCTAGTGTACCAGGAAAGTCCCGCTCCCACCACTGTATT
ACTTCCTCGAGACGCCCAGGCCGAAGTCCAAATGACTAATGCAGGTGCGCAGTTAGCTGGCGGCTCCACC
CTATGTCGTCACAGGCCTCGGCATAATATAAAACGCCTGATGATCAGAGGCCGAGGTATCCAGCTCAACG
ACGAGTCGGTGAGCTCTCCGCTTGGTCTACGACCAGACGGAATCTTTCAGATTGCCGGCTGCGGGAGATC
TTCCTTCACCCCTCGTCAGGCTGTTCTGACTTTGGAAAGTTCGTCTTCGCAACCCCGCTCGGGCGGAATC
GGGACCGTTCAATTTGTAGAGGAGTTTACTCCCTCTGTCTACTTCAACCCCTTCTCCGGATCTCCTGGGC
ACTACCCGGACGAGTTCATACCGAACTTCGACGCGATTAGCGAGTCAGTGGACGGCTACGATTGATGTCT
GGTGACGCGGCTGAGCTATCTCGGCTGCGACATCTAGACCACTGCCGCCGCTTTCGCTGCTTTGCCCGGG
AACTTATTGAGTTCATCTACTTCGAACTCCCCAAGGATCACCCTCAAGGTCCGGCCCACGGAGTGCGGAT
TACTATCGAAGGCAAAATAGACTCTCGCCTGCAACGAATTTTCTCCCAGCGGCCCGTGCTGATCGAGCGA
GACCAGGGAAACACCACGGTTAGTAATCAATTACGGGGTCATTAGTTCATAGCCCATATATGGAGTTAAC
TTGTTTATTGCAGCTTATAATGGTTACAAATAAAGCAATAGCATCACAAATTTCACAAATAAAGCATTTT
TTTCACTGCATTCTAGTTGTGGTTTGTCCAAACTCATCAATGTATCTTATCATGTCTGCTCGAAGCGGCC
GGCCGCCCCGACTCTAGAGTCGCGGCCTCATTAGGAAGTTCCTATACTTTCTAGAGAATAGGAACTTCTC
AGAAGAACTCGTCAAGAAGGCGATAGAAGGCGATGCGCTGCGAATCGGGAGCGGCGATACCGTAAAGCAC
GAGGAAGCGGTCAGCCCATTCGCCGCCAAGCTCTTCAGCAATATCACGGGTAGCCAACGCTATGTCCTGA
TAGCGGTCCGCCACACCCAGCCGGCCACAGTCGATGAATCCAGAAAAGCGGCCATTTTCCACCATGATAT
TCGGCAAGCAGGCATCGCCATGGGTCACGACGAGATCCTCGCCGTCGGGCATGCGCGCCTTGAGCCTGGC
GAACAGTTCGGCTGGCGCGAGCCCCTGATGCTCTTCGTCCAGATCATCCTGATCGACAAGACCGGCTTCC
ATCCGAGTACGTGCTCGCTCGATGCGATGTTTCGCTTGGTGGTCGAATGGGCAGGTAGCCGGATCAAGCG
TATGCAGCCGCCGCATTGCATCAGCCATGATGGATACTTTCTCGGCAGGAGCAAGGTGAGATGACAGGAG
ATCCTGCCCCGGCACTTCGCCCAATAGCAGCCAGTCCCTTCCCGCTTCAGTGACAACGTCGAGCACAGCT
GCGCAAGGAACGCCCGTCGTGGCCAGCCACGATAGCCGCGCTGCCTCGTCCTGCAGTTCATTCAGGGCAC
CGGACAGGTCGGTCTTGACAAAAAGAACCGGGCGCCCCTGCGCTGACAGCCGGAACACGGCGGCATCAGA
GCAGCCGATTGCCTGTTGTGCCCAGTCATAGCCGAATAGCCTCTCCACCCAAGCGGCCGGAGAACCTGCG
TGCAATCCATCTTGTTCAATGGCCGATCCCATAACACCCCTTGTATTACTGTTTATGTAAGCAGACAGTT
TTACTGTTCGTGATGATATATTTTTATCTTGTGCAATGTAACAGGTTGTGGCCATAGCGGGCCCGGGATT
TTCCTCCACGTCCCCGCATGTTAGAAGACTTCCCCTGCCCTCGGCTCTGGAAGTTCCTATACTTTCTAGA
GAATAGGAACTTCCCGCCAGAATGCGTTCGCACAGCCGCCAGCCGGTCACTCCGTTGATGGTTACTCGGA
ACAGCAGGGAGCCGTCGGGGTTGATCAGGCGCTCGTCGATAATTTTGTTGCCGTTCCACAGGGTCCCTGT
TACAGTGATCTTTTTGCCGTCGAACACGGCGATGCCTTCATACGGCCGTCCGAAATAGTCGATCATGTTC
GGCGTAACCCCGTCGATTACCAGTGTGCCATAGTGCAGGATCACCTTAAAGTGATGATCATCCACAGGGT
ACACCACCTTAAAAATTTTTTCGATCTGGCCCATTTGGTCGCCGCTCAGACCTTCATACGGGATGATGAC
ATGGATGTCGATCTTCAGCCCATTTTCACCGCTCAGGACAATCCTTTGGATCGGAGTTACGGACACCCCG
AGATTCTGAAACAAACTGGACACACCTCCCTGTTCAAGGACTTGGTCCAGGTTGTAGCCGGCTGTCTGTC
GCCAGTCCCCAACGAAATCTTCGAGTGTGAAGACCATGGATCCGGGCCCGGGGTTTTCTTCAACGTCTCC
AGCCTGCTTCAGCAGGCTGAAGTTAGTAGCTCCGCTTCCTCGAGCTCGAGATCTGGCGAAGGCGATGGGG
GTCTTGAAGGCGTGCTGGTACTCCACGATGCCCAGCTCGGTGTTGCTGTGCAGCTCCTCCACGCGGCGGA
AGGCGAACATGGGGCCCCCGTTCTGCAGGATGCTGGGGTGGATGGCGCTCTTGAAGTGCATGTGGCTGTC
CACCACGAAGCTGTAGTAGCCGCCGTCGCGCAGGCTGAAGGTGCGGGCGAAGCTGCCCACCAGCACGTTA
TCGCCCATGGGGTGCAGGTGCTCCACGGTGGCGTTGCTGCGGATGATCTTGTCGGTGAAGATCACGCTGT
CCTCGGGGAAGCCGGTGCCCACCACCTTGAAGTCGCCGATCACGCGGCCGGCCTCGTAGCGGTAGCTGAA
GCTCACGTGCAGCACGCCGCCGTCCTCGTACTTCTCGATGCGGGTGTTGGTGTAGCCGCCGTTGTTGATG
GCGTGCAGGAAGGGGTTCTCGTAGCCGCTGGGGTAGGTGCCGAAGTGGTAGAAGCCGTAGCCCATCACGT
GGCTCAGCAGGTAGGGGCTGAAGGTCAGGGCGCCTTTGGTGCTCTTCATCTTGTTGGTCATGCGGCCCTG
CTCGGGGGTGCCCTCTCCGCCGCCCACCAGCTCGAACTCCACGCCGTTCAGGGTGCCGGTGATGCGGCAC
TCGATCTTCATGGCGGGCATGGTGGCGACCGGTAGCGCTAGCGGCTTCGGTACCACGCGTTCGCTCGAAT
TAATCAATTCTTTGCCAAAATGATGAGACAGCACAATAACCAGCACGTTGCCCAGGAGCTGTAGGAAAAA
GAAGAAGGCATGAACATGGTTAGCAGAGGCTCTAGAGCCGCCGGTCACACGCCAGAAGCCGAACCCCGCC
CTGCCCCGTCCCCCCCGAAGGCAGCCGTCCCCCCGCGGACAGCCCCGAGGCTGGAGAGGGAGAAGGGGAC
GGCGGCGCGGCGACGCACGAAGGCCCTCCCCGCCCATTTCCTTCCTGCCGGGGCCCTCCCGGAGCCCCTC
AAGGCTTTCACGCAGCCACAGAAAAGAAACAAGCCGTCATTAAACCAAGCGCTAATTACAGCCCGGAGGA
GAAGGGCCGTCCCGCCCGCTCACCTGTGGGAGTAACGCGGTCAGTCAGAGCCGGGGCGGGCGGCGCGAGG
CGGCGCGGAGCGGGGCACGGGGCGAAGGCAACGCAGCGACTCCCGCCCGCCGCGCGCTTCGCTTTTTATA
GGGCCGCCGCCGCCGCCGCCTCGCCATAAAAGGAAACTTTCGGAGCGCGCCGCTCTGATTGGCTGCCGCC
GCACCTCTCCGCCTCGCCCCGCCCCGCCCCTCGCCCCGCCCCGCCCCGCCTGGCGCGCGCCCCCCCCCCC
CCCCCGCCCCCATCGCTGCACAAAATAATTAAAAAATAAATAAATACAAAATTGGGGGTGGGGAGGGGGG
GGAGATGGGGAGAGTGAAGCAGAACGTGGGGCTCACCTCGACCATGGTAATAGCGATGACTAATACGTAG
ATGTACTGCCAAGTAGGAAAGTCCCATAAGGTCATGTACTGGGCATAATGCCAGGCGGGCCATTTACCGT
CATTGACGTCAATAGGGGGCGTACTTGGCATATGATACACTTGATGTACTGCCAAGTGGGCAGTTTACCG
TAAATACTCCACCCATTGACGTCAATGGAAAGTCCCTATTGGCGTTACTATGGGAACATACGTCATTATT
GACGTCAATGGGCGGGGGTCGTTGGGCGGTCAGCCAGGCGGGCCATTTACCGTAAGTTATGTAACGCGGA
ACAACAACAATTGCATTCATTTTATGTTTCAGGTTCAGGGGGAGGTGTGGTCCTGCGATTCCATCGAGTG
CACCTACACCCTGCTGAAGACCCTATGCGGCCTAAGAGACCTGCTACCAATGAATTAAAAAAAAATGATT
AATAAAAAATCACTTACTTGAAATCAGCAATAAGGTCTCTGTTGAAATTTTCTCCCAGCAGCACCTCACT
TCCCTCTTCCCAACTCTGGTATTCTAAACCCCGTTCAGCGGCATACTTTCTCCATACTTTAAAGGGGATG
TCAAATTTTAGCTCCTCTCCTGTACCCACAATCTTCATGTCTTTCTTCCCAGATGACCAAGAGAGTCCGG
CTCAGTGACTCCTTCAACCCTGTCTACCCCTATGAAGATGAAAGCACCTCCCAACACCCCTTTATAAACC
CAGGGTTTATTTCCCCAAATGGCTTCACACAAAGCCCAGACGGAGTTCTTACTTTAAAATGTTTAACCCC
ACTAACAACCACAGGCGGATCTCTACAGCTAAAAGTGGGAGGGGGACTTACAGTGGATGACACTGATGGT
ACCTTACAAGAAAACATACGTGCTACAGCACCCATTACTAAAAATAATCACTCTGTAGAACTATCCATTG
GAAATGGATTAGAAACTCAAAACAATAAACTATGTGCCAAATTGGGAAATGGGTTAAAATTTAACAACGG
TGACATTTGTATAAAGGATAGTATTAACACCTTATGGACTGGAATAAACCCTCCACCTAACTGTCAAATT
GTGGAAAACACTAATACAAATGATGGCAAACTTACTTTAGTATTAGTAAAAAATGGAGGGCTTGTTAATG
GCTACGTGTCTCTAGTTGGTGTATCAGACACTGTGAACCAAATGTTCACACAAAAGACAGCAAACATCCA
ATTAAGATTATATTTTGACTCTTCTGGAAATCTATTAACTGAGGAATCAGACTTAAAAATTCCACTTAAA
AATAAATCTTCTACAGCGACCAGTGAAACTGTAGCCAGCAGCAAAGCCTTTATGCCAAGTACTACAGCTT
ATCCCTTCAACACCACTACTAGGGATAGTGAAAACTACATTCATGGAATATGTTACTACATGACTAGTTA
TGATAGAAGTCTATTTCCCTTGAACATTTCTATAATGCTAAACAGCCGTATGATTTCTTCCAATGTTGCC
TATGCCATACAATTTGAATGGAATCTAAATGCAAGTGAATCTCCAGAAAGCAACATAGCTACGCTGACCA
CATCCCCCTTTTTCTTTTCTTACATTACAGAAGACGACAACTAAAATAAAGTTTAAGTGTTTTTATTTAA
AATCACAAAATTCGAGTAGTTATTTTGCCTCCACCTTCCCATTTGACAGAATACACAGTCCTTTCTCCCC
GGCTGGCCTTAAAAAGCATCATATCATGGGTAACAGACATATTCTTAGGTGTTATATTCCACACGGTTTC
CTGTCGAGCCAAACGCTCATCAGTGATATTAATAAACTCCCCGGGCAGCTCACTTAAGTTCATGTCGCTG
TCCAGCTGCTGAGCCACAGGCTGCTGTCCAACTTGCGGTTGCTTAACGGGCGGCGAAGGAGAAGTCCACG
CCTACATGGGGGTAGAGTCATAATCGTGCATCAGGATAGGGCGGTGGTGCTGCAGCAGCGCGCGAATAAA
CTGCTGCCGCCGCCGCTCCGTCCTGCAGGAATACAACATGGCAGTGGTCTCCTCAGCGATGATTCGCACC
GCCCGCAGCATAAGGCGCCTTGTCCTCCGGGCACAGCAGCGCACCCTGATCTCACTTAAATCAGCACAGT
AACTGCAGCACAGCACCACAATATTGTTCAAAATCCCACAGTGCAAGGCGCTGTATCCAAAGCTCATGGC
GGGGACCACAGAACCCACGTGGCCATCATACCACAAGCGCAGGTAGATTAAGTGGCGACCCCTCATAAAC
ACGCTGGACATAAACATTACCTCTTTTGGCATGTTGTAATTCACCACCTCCCGGTACCATATAAACCTCT
GATTAAACATGGCGCCATCCACCACCATCCTAAACCAGCTGGCCAAAACCTGCCCGCCGGCTATACACTG
CAGGGAACCGGGACTGGAACAATGACAGTGGAGAGCCCAGGACTCGTAACCATGGATCATCATGCTCGTC
ATGATATCAATGTTGGCACAACACAGGCACACGTGCATACACTTCCTCAGGATTACAAGCTCCTCCCGCG
TTAGAACCATATCCCAGGGAACAACCCATTCCTGAATCAGCGTAAATCCCACACTGCAGGGAAGACCTCG
CACGTAACTCACGTTGTGCATTGTCAAAGTGTTACATTCGGGCAGCAGCGGATGATCCTCCAGTATGGTA
GCGCGGGTTTCTGTCTCAAAAGGAGGTAGACGATCCCTACTGTACGGAGTGCGCCGAGACAACCGAGATC
GTGTTGGTCGTAGTGTCATGCCAAATGGAACGCCGGACGTAGTCATTCTCGTATTTTGTATAGCAAAACG
CGGCCCTGGCAGAACACACTCTTCTTCGCCTTCTATCCTGCCGCTTAGCGTGTTCCGTGTGATAGTTCAA
GTACAGCCACACTCTTAAGTTGGTCAAAAGAATGCTGGCTTCAGTTGTAATCAAAACTCCATCGCATCTA
ATTGTTCTGAGGAAATCATCCACGGTAGCATATGCAAATCCCAACCAAGCAATGCAACTGGATTGCGTTT
CAAGCAGGAGAGGAGAGGGAAGAGACGGAAGAACCATGTTAATTTTTATTCCAAACGATCTCGCAGTACT
TCAAATTGTAGATCGCGCAGATGGCATCTCTCGCCCCCACTGTGTTGGTGAAAAAGCACAGCTAAATCAA
AAGAAATGCGATTTTCAAGGTGCTCAACGGTGGCTTCCAACAAAGCCTCCACGCGCACATCCAAGAACAA
AAGAATACCAAAAGAAGGAGCATTTTCTAACTCCTCAATCATCATATTACATTCCTGCACCATTCCCAGA
TAATTTTCAGCTTTCCAGCCTTGAATTATTCGTGTCAGTTCTTGTGGTAAATCCAATCCACACATTACAA
ACAGGTCCCGGAGGGCGCCCTCCACCACCATTCTTAAACACACCCTCATAATGACAAAATATCTTGCTCC
TGTGTCACCTGTAGCGAATTGAGAATGGCAACATCAATTGACATGCCCTTGGCTCTAAGTTCTTCTTTAA
GTTCTAGTTGTAAAAACTCTCTCATATTATCACCAAACTGCTTAGCCAGAAGCCCCCCGGGAACAAGAGC
AGGGGACGCTACAGTGCAGTACAAGCGCAGACCTCCCCAATTGGCTCCAGCAAAAACAAGATTGGAATAA
GCATATTGGGAACCACCAGTAATATCATCGAAGTTGCTGGAAATATAATCAGGCAGAGTTTCTTGTAGAA
ATTGAATAAAAGAAAAATTTGCCAAAAAAACATTCAAAACCTCTGGGATGCAAATGCAATAGGTTACCGC
GCTGCGCTCCAACATTGTTAGTTTTGAATTAGTCTGCAAAAATAAAAAAAAAACAAGCGTCATATCATAG
TAGCCTGACGAACAGGTGGATAAATCAGTCTTTCCATCACAAGACAAGCCACAGGGTCTCCAGCTCGACC
CTCGTAAAACCTGTCATCGTGATTAAACAACAGCACCGAAAGTTCCTCGCGGTGACCAGCATGAATAAGT
CTTGATGAAGCATACAATCCAGACATGTTAGCATCAGTTAAGGAGAAAAAACAGCCAACATAGCCTTTGG
GTATAATTATGCTTAATCGTAAGTATAGCAAAGCCACCCCTCGCGGATACAAAGTAAAAGGCACAGGAGA
ATAAAAAATATAATTATTTCTCTGCTGCTGTTTAGGCAACGTCGCCCCCGGTCCCTCTAAATACACATAC
AAAGCCTCATCAGCCATGGCTTACCAGAGAAAGTACAGCGGGCACACAAACCACAAGCTCTAAAGTCACT
CTCCAACCTCTCCACAATATATATACACAAGCCCTAAACTGACGTAATGGGACTAAAGTGTAAAAAATCC
CGCCAAACCCAACACACACCCCGAAACTGCGTCACCAGGGAAAAGTACAGTTTCACTTCCGCAATCCCAA
CAAGCGTCACTTCCTCTTTCTCACGGTACGTCACATCCCATTAACTTACAACGTCATTTTCCCACGGCCG
CGCCGCCCCTTTTAACCGTTAACCCCACAGCCAATCACCACACGGCCCACACTTTTTAAAATCACCTCAT
TTACATATTGGCACCATTCCATCTATAAGGTATATTATTGATGATGGCCAAGCTATTTAGGTGACACTAT
AGAATACTCAAGCTATGCATCAAGCTTGGTACCGAGCTCGGATCCACTAGTAACGGCCGCCAGTGTGCTG
GAATTCGCCCTTGTTTAAACGCGATCGCTTGAGATCGTTTTGGTCTGCGCGTAATCTCTTGCTCTGAAAA
CGAAAAAACCGCCTTGCAGGGCGGTTTTTCGAAGGTTCTCTGAGCTACCAACTCTTTGAACCGAGGTAAC
TGGCTTGGAGGAGCGCAGTCACCAAAACTTGTCCTTTCAGTTTAGCCTTAACCGGCGCATGACTTCAAGA
CTAACTCCTCTAAATCAATTACCAGTGGCTGCTGCCAGTGGTGCTTTTGCATGTCTTTCCGGGTTGGACT
CAAGACGATAGTTACCGGATAAGGCGCAGCGGTCGGACTGAACGGGGGGTTCGTGCATACAGTCCAGCTT
GGAGCGAACTGCCTACCCGGAACTGAGTGTCAGGCGTGGAATGAGACAAACGCGGCCATAACAGCGGAAT
GACACCGGTAAACCGAAAGGCAGGAACAGGAGAGCGCACGAGGGAGCCGCCAGGGGGAAACGCCTGGTAT
CTTTATAGTCCTGTCGGGTTTCGCCACCACTGATTTGAGCGTCAGATTTCGTGATGCTTGTCAGGGGGGC
GGAGCCTATGGAAAAACGGCTTTGCCGCGGCCCTCTCACTTCCCTGTTAAGTATCTTCCTGGCATCTTCC
AGGAAATCTCCGCCCCGTTCGTAAGCCATTTCCGCTCGCCGCAGTCGAACGACCGAGCGTAGCGAGTCAG
TGAGCGAGGAAGCGGAATATATCCTGTATCACATATTCTGCTGACGCACCGGTGCAGCCTTTTTTCTCCT
GCCACATGAAGCACTTCACTGACACCCTCATCAGTGCCAACATAGTAAGCCAGTATACACTCCGCTAGCG
CGATCGCTTAATTAATTTAAATCCTGCAGGGTTTAAACGGCCGGCCTAGGGATAACAGGGTAATCGTAAC
TATAACGGTCCTAAGGTAGCGAATGATGTCCGGCGGTGCTTTTGCCGTTACGCACCACCCCGTCAGTAGC
TGAACAGGAGGGACAGCTGATAGAAACAGAAGCCACTGGAGCACCTCAAAAACACCATCATACACTAAAT
CAGTAAGTTGGCAGCATCACCCGACGCACTTTGCGCCGAATAAATACCTGTGACGGAAGATCACTTCGCA
GAATAAATAAATCCTGGTGTCCCTGTTGATACCGGGAAGCCCTGGGCCAACTTTTGGCGAAAATGAGACG
TTGATCGGCACGTAAGAGGTTCCAACTTTCACCATAATGAAATAAGATCACTACCGGGCGTATTTTTTGA
GTTATCGAGATTTTCAGGAGCTAAGGAAGCTAAAATGGAGAAAAAAATCACTGGATATACCACCGTTGAT
ATATCCCAATGGCATCGTAAAGAACATTTTGAGGCATTTCAGTCAGTTGCTCAATGTACCTATAACCAGA
CCGTTCAGCTGGATATTACGGCCTTTTTAAAGACCGTAAAGAAAAATAAGCACAAGTTTTATCCGGCCTT
TATTCACATTCTTGCCCGCCTGATGAATGCTCATCCGGAATTCCGTATGGCAATGAAAGACGGTGAGCTG
GTGATATGGGATAGTGTTCACCCTTGTTACACCGTTTTCCATGAGCAAACTGAAACGTTTTCATCGCTCT
GGAGTGAATACCACGACGATTTCCGGCAGTTTCTACACATATATTCGCAAGATGTGGCGTGTTACGGTGA
AAACCTGGCCTATTTCCCTAAAGGGTTTATTGAGAATATGTTTTTCGTCTCAGCCAATCCCTGGGTGAGT
TTCACCAGTTTTGATTTAAACGTGGCCAATATGGACAACTTCTTCGCCCCCGTTTTCACCATGGGCAAAT
ATTATACGCAAGGCGACAAGGTGCTGATGCCGCTGGCGATTCAGGTTCATCATGCCGTCTGTGATGGCTT
CCATGTCGGCAGAATGCTTAATGAATTACAACAGTACTGCGATGAGTGGCAGGGCGGGGCGTAACCTGCA
GGTTAATTAAGGAAGGGCGAATTCTGCAGATATCCATCACACTGGCGGCCGCTCGAGCATGCATCTAGAG
GGCCCAATTCGCCCTATAGTGAGTCGTATTACAATTCACTGGCC (SEQ ID NO: 175)
The nucleotide sequence of PS2:
CATCATCAATAATATACCTTATAGATGGAATGGTGCCAATATGTAAATGAGGTGATTTTAAAAAGTGTGG
GCCGTGTGGTGATTGGCTGTGGGGTTAACGGTTAAAAGGGGCGGCGCGGCCGTGGGAAAATGACGTTTTA
TGGGGGTGGAGTTTTTTTGCAAGTTGTCGCGGGAAATGATAACTTCGTATAGCATACATTATACGAAGTT
ATTAGACTTTGACCCATTACGTGGAGGTTTCGATTACCGTGTTTTTTACCTGAATTTCCGCGTACCGTGT
CAAAGTCTTCTGTTTTTACGTAGGTGTCAGCTGATCGCTAGGGTATTTATAACTTCGTATAGCATACATT
ATACGAAGTTATGGAATGTTTATGCCTTACCAGTGTAACATGAATCATGTGAAAGTGTTGTTGGAACCAG
ATGCCTTTTCCAGAATGAGCCTAACAGGAATCTTTGACATGAACACGCAAATCTGGAAGATCCTGAGGTA
TGATGATACGAGATCGAGGGTGCGCGCATGCGAATGCGGAGGCAAGCATGCCAGGTTCCAGCCGGTGTGT
GTAGATGTGACCGAAGATCTCAGACCGGATCATTTGGTTATTGCCCGCACTGGAGCAGAGTTCGGATCCA
GTGGAGAAGAAACTGACTAAGGTGAGTATTGGGAAAACTTTGGGGTGGGATTTTCAGATGGACAGATTGA
GTAAAAATTTGTTTTTTCTGTCTTGCAGCTGACATGAGTGGAAATGCTTCTTTTAAGGGGGGAGTCTTCA
GCCCTTATCTGACAGGGCGTCTCCCATCCTGGGCAGGAGTTCGTCAGAATGTTATGGGATCTACTGTGGA
TGGAAGACCCGTTCAACCCGCCAATTCTTCAACGCTGACCTATGCTACTTTAAGTTCTTCACCTTTGGAC
GCAGCTGCAGCCGCTGCCGCCGCCTCTGTCGCCGCTAACACTGTGCTTGGAATGGGTTACTATGGAAGCA
TCGTGGCTAATTCCACTTCCTCTAATAACCCTTCTACACTGACTCAGGACAAGTTACTTGTCCTTTTGGC
CCAGCTGGAGGCTTTGACCCAACGTCTGGGTGAACTTTCTCAGCAGGTGGCCGAGTTGCGAGTACAAACT
GAGTCTGCTGTCGGCACGGCAAAGTCTAAATAAAAAAAATTCCAGAATCAATGAATAAATAAACGAGCTT
GTTGTTGATTTAAAATCAAGTGTTTTTATTTCATTTTTCGCGCACGGTATGCCCTGGACCACCGATCTCG
ATCATTGAGAACTCGGTGGATTTTTTCCAGAATCCTATAGAGGTGGGATTGAATGTTTAGATACATGGGC
ATTAGGCCGTCTTTGGGGTGGAGATAGCTCCATTGAAGGGATTCATGCTCCGGGGTAGTGTTGTAAATCA
CCCAGTCATAACAAGGTCGCAGTGCATGGTGTTGCACAATATCTTTTAGAAGTAGGCTGATTGCCACAGA
TAAGCCCTTGGTGTAGGTGTTTACAAACCGGTTGAGCTGGGAGGGGTGCATTCGAGGTGAAATTATGTGC
ATTTTGGATTGGATTTTTAAGTTGGCAATATTGCCGCCAAGATCCCGTCTTGGGTTCATGTTATGAAGGA
CTACCAAGACGGTGTATCCGGTACATTTAGGAAATTTATCGTGCAGCTTGGATGGAAAAGCGTGGAAAAA
TTTGGAGACACCCTTGTGTCCTCCGAGATTTTCCATGCACTCATCCATGATAATAGCAATGGGGCCGTGG
GCAGCGGCGCGGGCAAACACGTTCCGTGGGTCTGACACATCATAGTTATGTTCCTGAGTTAAATCATCAT
AAGCCATTTTAATGAATTTGGGGCGGAGCGTACCAGATTGGGGTATGAATGTTCCTTCGGGCCCCGGAGC
ATAGTTCCCCTCACAGATTTGCATTTCCCAAGCTTTCAGTTCTGAGGGTGGAATCATGTCCACCTGGGGG
GCTATGAAGAACACCGTTTCGGGGGCGGGGGTGATTAGTTGGGATGATAGCAAGTTTCTGAGCAATTGAG
ATTTGCCACATCCGGTGGGGCCATAAATAATTCCGATTACAGGTTGCAGGTGGTAGTTTAGGGAACGGCA
ACTGCCGTCTTCTCGAAGCAAGGGGGCCACCTCGTTCATCATTTCCCTTACATGCATATTTTCCCGCACC
AAATCCATTAGGAGGCGCTCTCCTCCTAGTGATAGAAGTTCTTGTAGTGAGGAAAAGTTTTTCAGCGGTT
TTAGACCGTCAGCCATGGGCATTTTGGAAAGAGTTTGCTGCAAAAGTTCTAGTCTGTTCCACAGTTCAGT
GATGTGTTCTATGGCATCTCGATCCAGCAGACCTCCTCGTTTCGCGGGTTTGGACGGCTCCTGGAGTAGG
GTATGAGACGATGGGCGTCCAGCGCTGCCAGGGTTCGGTCCTTCCAGGGTCTCAGTGTTCGAGTCAGGGT
TGTTTCCGTCACAGTGAAGGGGTGTGCGCCTGCTTGGGCGCTTGCCAGGGTGCGCTTCAGACTCATTCTG
CTGGTGGAGAACTTCTGTCGCTTGGCGCCCTGTATGTCGGCCAAGTAGCAGTTTACCATGAGTTCGTAGT
TGAGCGCCTCGGCTGCGTGGCCTTTGGCGCGGAGCTTACCTTTGGAAGTTTTCTTGCATACCGGGCAGTA
TAGGCATTTCAGCGCATACAGCTTGGGCGCAAGGAAAATGGATTCTGGGGAGTATGCATCCGCGCCGCAG
GAGGCGCAAACAGTTTCACATTCCACCAGCCAGGTTAAATCCGGTTCATTGGGGTCAAAAACAAGTTTTC
CGCCATATTTTTTGATGCGTTTCTTACCTTTGGTCTCCATAAGTTCGTGTCCTCGTTGAGTGACAAACAG
GCTGTCCGTATCTCCGTAGACTGATTTTACAGGCCTCTTCTCCAGTGGAGTGCCTCGGTCTTCTTCGTAC
AGGAACTCTGACCACTCTGATACAAAGGCGCGCGTCCAGGCCAGCACAAAGGAGGCTATGTGGGAGGGGT
AGCGATCGTTGTCAACCAGGGGGTCCACCTTTTCCAAAGTATGCAAACACATGTCACCCTCTTCAACATC
CAGGAATGTGATTGGCTTGTAGGTGTATTTCACGTGACCTGGGGTCCCCGCTGGGGGGGTATAAAAGGGG
GCGGTTCTTTGCTCTTCCTCACTGTCTTCCGGATCGCTGTCCAGGAACGTCAGCTGTTGGGGTAGGTATT
CCCTCTCGAAGGCGGGCATGACCTCTGCACTCAGGTTGTCAGTTTCTAAGAACGAGGAGGATTTGATATT
GACAGTGCCGGTTGAGATGCCTTTCATGAGGTTTTCGTCCATTTGGTCAGAAAACACAATTTTTTTATTG
TCAAGTTTGGTGGCAAATGATCCATACAGGGCGTTGGATAAAAGTTTGGCAATGGATCGCATGGTTTGGT
TCTTTTCCTTGTCCGCGCGCTCTTTGGCGGCGATGTTGAGTTGGACATACTCGCGTGCCAGGCACTTCCA
TTCGGGGAAGATAGTTGTTAATTCATCTGGCACGATTCTCACTTGCCACCCTCGATTATGCAAGGTAATT
AAATCCACACTGGTGGCCACCTCGCCTCGAAGGGGTTCATTGGTCCAACAGAGCCTACCTCCTTTCCTAG
AACAGAAAGGGGGAAGTGGGTCTAGCATAAGTTCATCGGGAGGGTCTGCATCCATGGTAAAGATTCCCGG
AAGTAAATCCTTATCAAAATAGCTGATGGGAGTGGGGTCATCTAAGGCCATTTGCCATTCTCGAGCTGCC
AGTGCGCGCTCATATGGGTTAAGGGGACTGCCCCAGGGCATGGGATGGGTGAGAGCAGAGGCATACATGC
CACAGATGTCATAGACGTAGATGGGATCCTCAAAGATGCCTATGTAGGTTGGATAGCATCGCCCCCCTCT
GATACTTGCTCGCACATAGTCATATAGTTCATGTGATGGCGCTAGCAGCCCCGGACCCAAGTTGGTGCGA
TTGGGTTTTTCTGTTCTGTAGACGATCTGGCGAAAGATGGCGTGAGAATTGGAAGAGATGGTGGGTCTTT
GAAAAATGTTGAAATGGGCATGAGGTAGACCTACAGAGTCTCTGACAAAGTGGGCATAAGATTCTTGAAG
CTTGGTTACCAGTTCGGCGGTGACAAGTACGTCTAGGGCGCAGTAGTCAAGTGTTTCTTGAATGATGTCA
TAACCTGGTTGGTTTTTCTTTTCCCACAGTTCGCGGTTGAGAAGGTATTCTTCGCGATCCTTCCAGTACT
CTTCTAGCGGAAACCCGTCTTTGTCTGCACGGTAAGATCCTAGCATGTAGAACTGATTAACTGCCTTGTA
AGGGCAGCAGCCCTTCTCTACGGGTAGAGAGTATGCTTGAGCAGCTTTTCGTAGCGAAGCGTGAGTAAGG
GCAAAGGTGTCTCTGACCATGACTTTGAGAAATTGGTATTTGAAGTCCATGTCGTCACAGGCTCCCTGTT
CCCAGAGTTGGAAGTCTACCCGTTTCTTGTAGGCGGGGTTGGGCAAAGCGAAAGTAACATCATTGAAGAG
AATCTTACCGGCTCTGGGCATAAAATTGCGAGTGATGCGGAAAGGCTGTGGTACTTCCGCTCGATTGTTG
ATCACCTGGGCAGCTAGGACGATTTCGTCGAAACCGTTGATGTTGTGTCCTACGATGTATAATTCTATGA
AACGCGGCGTGCCTCTGACGTGAGGTAGCTTACTGAGCTCATCAAAGGTTAGGTCTGTGGGGTCAGATAA
GGCGTAGTGTTCGAGAGCCCATTCGTGCAGGTGAGGATTTGCATGTAGGAATGATGACCAAAGATCTACC
GCCAGTGCTGTTTGTAACTGGTCCCGATACTGACGAAAATGCCGGCCAATTGCCATTTTTTCTGGAGTGA
CACAGTAGAAGGTTCTGGGGTCTTGTTGCCATCGATCCCACTTGAGTTTAATGGCTAGATCGTGGGCCAT
GTTGACGAGACGCTCTTCTCCTGAGAGTTTCATGACCAGCATGAAAGGAACTAGTTGTTTGCCAAAGGAT
CCCATCCAGGTGTAAGTTTCCACATCGTAGGTCAGGAAGAGTCTTTCTGTGCGAGGATGAGAGCCGATCG
GGAAGAACTGGATTTCCTGCCACCAGTTGGAGGATTGGCTGTTGATGTGATGGAAGTAGAAGTTTCTGCG
GCGCGCCGAGCATTCGTGTTTGTGCTTGTACAGACGGCCGCAGTAGTCGCAGCGTTGCACGGGTTGTATC
TCGTGAATGAGCTGTACCTGGCTTCCCTTGACGAGAAATTTCAGTGGGAAGCCGAGGCCTGGCGATTGTA
TCTCGTGCTCTTCTATATTCGCTGTATCGGCCTGTTCATCTTCTGTTTCGATGGTGGTCATGCTGACGAG
CCCCCGCGGGAGGCAAGTCCAGACCTCGGCGCGGGAGGGGCGGAGCTGAAGGACGAGAGCGCGCAGGCTG
GAGCTGTCCAGAGTCCTGAGACGCTGCGGACTCAGGTTAGTAGGTAGGGACAGAAGATTAACTTGCATGA
TCTTTTCCAGGGCGTGCGGGAGGTTCAGATGGTACTTGATTTCCACAGGTTCGTTTGTAGAGACGTCAAT
GGCTTGCAGGGTTCCGTGTCCTTTGGGCGCCACTACCGTACCTTTGTTTTTTCTTTTGATCGGTGGTGGC
TCTCTTGCTTCTTGCATGCTCAGAAGCGGTGACGGGGACGCGCGCCGGGCGGCAGCGGTTGTTCCGGACC
CGGGGGCATGGCTGGTAGTGGCACGTCGGCGCCGCGCACGGGCAGGTTCTGGTATTGCGCTCTGAGAAGA
CTTGCGTGCGCCACCACGCGTCGATTGACGTCTTGTATCTGACGTCTCTGGGTGAAAGCTACCGGCCCCG
TGAGCTTGAACCTGAAAGAGAGTTCAACAGAATCAATTTCGGTATCGTTAACGGCAGCTTGTCTCAGTAT
TTCTTGTACGTCACCAGAGTTGTCCTGGTAGGCGATCTCCGCCATGAACTGCTCGATTTCTTCCTCCTGA
AGATCTCCGCGACCCGCTCTTTCGACGGTGGCCGCGAGGTCATTGGAGATACGGCCCATGAGTTGGGAGA
ATGCATTCATGCCCGCCTCGTTCCAGACGCGGCTGTAAACCACGGCCCCCTCGGAGTCTCTTGCGCGCAT
CACCACCTGAGCGAGGTTAAGCTCCACGTGTCTGGTGAAGACCGCATAGTTGCATAGGCGCTGAAAAAGG
TAGTTGAGTGTGGTGGCAATGTGTTCGGCGACGAAGAAATACATGATCCATCGTCTCAGCGGCATTTCGC
TAACATCGCCCAGAGCTTCCAAGCGCTCCATGGCCTCGTAGAAGTCCACGGCAAAATTAAAAAACTGGGA
GTTTCGCGCGGACACGGTCAATTCCTCCTCGAGAAGACGGATGAGTTCGGCTATGGTGGCCCGTACTTCG
CGTTCGAAGGCTCCCGGGATCTCTTCTTCCTCTTCTATCTCTTCTTCCACTAACATCTCTTCTTCGTCTT
CAGGCGGGGGCGGAGGGGGCACGCGGCGACGTCGACGGCGCACGGGCAAACGGTCGATGAATCGTTCAAT
GACCTCTCCGCGGCGGCGGCGCATGGTTTCAGTGACGGCGCGGCCGTTCTCGCGCGGTCGCAGAGTAAAA
ACACCGCCGCGCATCTCCTTAAAGTGGTGACTGGGAGGTTCTCCGTTTGGGAGGGAGAGGGCGCTGATTA
TACATTTTATTAATTGGCCCGTAGGGACTGCGCGCAGAGATCTGATCGTGTCAAGATCCACGGGATCTGA
AAACCTTTCGACGAAAGCGTCTAACCAGTCACAGTCACAAGGTAGGCTGAGTACGGCTTCTTGTGGGCGG
GGGTGGTTATGTGTTCGGTCTGGGTCTTCTGTTTCTTCTTCATCTCGGGAAGGTGAGACGATGCTGCTGG
TGATGAAATTAAAGTAGGCAGTTCTAAGACGGCGGATGGTGGCGAGGAGCACCAGGTCTTTGGGTCCGGC
TTGCTGGATACGCAGGCGATTGGCCATTCCCCAAGCATTATCCTGACATCTAGCAAGATCTTTGTAGTAG
TCTTGCATGAGCCGTTCTACGGGCACTTCTTCCTCACCCGTTCTGCCATGCATACGTGTGAGTCCAAATC
CGCGCATTGGTTGTACCAGTGCCAAGTCAGCTACGACTCTTTCGGCGAGGATGGCTTGCTGTACTTGGGT
AAGGGTGGCTTGAAAGTCATCAAAATCCACAAAGCGGTGGTAAGCCCCTGTATTAATGGTGTAAGCACAG
TTGGCCATGACTGACCAGTTAACTGTCTGGTGACCAGGGCGCACGAGCTCGGTGTATTTAAGGCGCGAAT
AGGCGCGGGTGTCAAAGATGTAATCGTTGCAGGTGCGCACCAGATACTGGTACCCTATAAGAAAATGCGG
CGGTGGTTGGCGGTAGAGAGGCCATCGTTCTGTAGCTGGAGCGCCAGGGGCGAGGTCTTCCAACATAAGG
CGGTGATAGCCGTAGATGTACCTGGACATCCAGGTGATTCCTGCGGCGGTAGTAGAAGCCCGAGGAAACT
CGCGTACGCGGTTCCAAATGTTGCGTAGCGGCATGAAGTAGTTCATTGTAGGCACGGTTTGACCAGTGAG
GCGCGCGCAGTCATTGATGCTCTATAGACACGGAGAAAATGAAAGCGTTCAGCGACTCGACTCCGTAGCC
TGGAGGAACGTGAACGGGTTGGGTCGCGGTGTACCCCGGTTCGAGACTTGTACTCGAGCCGGCCGGAGCC
GCGGCTAACGTGGTATTGGCACTCCCGTCTCGACCCAGCCTACAAAAATCCAGGATACGGAATCGAGTCG
TTTTGCTGGTTTCCGAATGGCAGGGAAGTGAGTCCTATTTTTTTTTTTTTTGCCGCTCAGATGCATCCCG
TGCTGCGACAGATGCGCCCCCAACAACAGCCCCCCTCGCAGCAGCAGCAGCAGCAACCACAAAAGGCTGT
CCCTGCAACTACTGCAACTGCCGCCGTGAGCGGTGCGGGACAGCCCGCCTATGATCTGGACTTGGAAGAG
GGCGAAGGACTGGCACGTCTAGGTGCGCCTTCGCCCGAGCGGCATCCGCGAGTTCAACTGAAAAAAGATT
CTCGCGAGGCGTATGTGCCCCAACAGAACCTATTTAGAGACAGAAGCGGCGAGGAGCCGGAGGAGATGCG
AGCTTCCCGCTTTAACGCGGGTCGTGAGCTGCGTCACGGTTTGGACCGAAGACGAGTGTTGCGAGACGAG
GATTTCGAAGTTGATGAAGTGACAGGGATCAGTCCTGCCAGGGCACACGTGGCTGCAGCCAACCTTGTAT
CGGCTTACGAGCAGACAGTAAAGGAAGAGCGTAACTTCCAAAAGTCTTTTAATAATCATGTGCGAACCCT
GATTGCCCGCGAAGAAGTTACCCTTGGTTTGATGCATTTGTGGGATTTGATGGAAGCTATCATTCAGAAC
CCTACTAGCAAACCTCTGACCGCCCAGCTGTTTCTGGTGGTGCAACACAGCAGAGACAATGAGGCTTTCA
GAGAGGCGCTGCTGAACATCACCGAACCCGAGGGGAGATGGTTGTATGATCTTATCAACATTCTACAGAG
TATCATAGTGCAGGAGCGGAGCCTGGGCCTGGCCGAGAAGGTAGCTGCCATCAATTACTCGGTTTTGAGC
TTGGGAAAATATTACGCTCGCAAAATCTACAAGACTCCATACGTTCCCATAGACAAGGAGGTGAAGATAG
ATGGGTTCTACATGCGCATGACGCTCAAGGTCTTGACCCTGAGCGATGATCTTGGGGTGTATCGCAATGA
CAGAATGCATCGCGCGGTTAGCGCCAGCAGGAGGCGCGAGTTAAGCGACAGGGAACTGATGCACAGTTTG
CAAAGAGCTCTGACTGGAGCTGGAACCGAGGGTGAGAATTACTTCGACATGGGAGCTGACTTGCAGTGGC
AGCCTAGTCGCAGGGCTCTGAGCGCCGCGACGGCAGGATGTGAGCTTCCTTACATAGAAGAGGCGGATGA
AGGCGAGGAGGAAGAGGGCGAGTACTTGGAAGACTGATGGCACAACCCGTGTTTTTTGCTAGATGGAACA
GCAAGCACCGGATCCCGCAATGCGGGCGGCGCTGCAGAGCCAGCCGTCCGGCATTAACTCCTCGGACGAT
TGGACCCAGGCCATGCAACGTATCATGGCGTTGACGACTCGCAACCCCGAAGCCTTTAGACAGCAACCCC
AGGCCAACCGTCTATCGGCCATCATGGAAGCTGTAGTGCCTTCCCGATCTAATCCCACTCATGAGAAGGT
CCTGGCCATCGTGAACGCGTTGGTGGAGAACAAAGCTATTCGTCCAGATGAGGCCGGACTGGTATACAAC
GCTCTCTTAGAACGCGTGGCTCGCTACAACAGTAGCAATGTGCAAACCAATTTGGACCGTATGATAACAG
ATGTACGCGAAGCCGTGTCTCAGCGCGAAAGGTTCCAGCGTGATGCCAACCTGGGTTCGCTGGTGGCGTT
AAATGCTTTCTTGAGTACTCAGCCTGCTAATGTGCCGCGTGGTCAACAGGATTATACTAACTTTTTAAGT
GCTTTGAGACTGATGGTATCAGAAGTACCTCAGAGCGAAGTGTATCAGTCCGGTCCTGATTACTTCTTTC
AGACTAGCAGACAGGGCTTGCAGACGGTAAATCTGAGCCAAGCTTTTAAAAACCTTAAAGGTTTGTGGGG
AGTGCATGCCCCGGTAGGAGAAAGAGCAACCGTGTCTAGCTTGTTAACTCCGAACTCCCGCCTGTTATTA
CTGTTGGTAGCTCCTTTCACCGACAGCGGTAGCATCGACCGTAATTCCTATTTGGGTTACCTACTAAACC
TGTATCGCGAAGCCATAGGGCAAAGTCAGGTGGACGAGCAGACCTATCAAGAAATTACCCAAGTCAGTCG
CGCTTTGGGACAGGAAGACACTGGCAGTTTGGAAGCCACTCTGAACTTCTTGCTTACCAATCGGTCTCAA
AAGATCCCTCCTCAATATGCTCTTACTGCGGAGGAGGAGAGGATCCTTAGATATGTGCAGCAGAGCGTGG
GATTGTTTCTGATGCAAGAGGGGGCAACTCCGACTGCAGCACTGGACATGACAGCGCGAAATATGGAGCC
CAGCATGTATGCCAGTAACCGACCTTTCATTAACAAACTGCTGGACTACTTGCACAGAGCTGCCGCTATG
AACTCTGATTATTTCACCAATGCCATCTTAAACCCGCACTGGCTGCCCCCACCTGGTTTCTACACGGGCG
AATATGACATGCCCGACCCTAATGACGGATTTCTGTGGGACGACGTGGACAGCGATGTTTTTTCACCTCT
TTCTGATCATCGCACGTGGAAAAAGGAAGGCGGTGATAGAATGCATTCTTCTGCATCGCTGTCCGGGGTC
ATGGGTGCTACCGCGGCTGAGCCCGAGTCTGCAAGTCCTTTTCCTAGTCTACCCTTTTCTCTACACAGTG
TACGTAGCAGCGAAGTGGGTAGAATAAGTCGCCCGAGTTTAATGGGCGAAGAGGAGTACCTAAACGATTC
CTTGCTCAGACCGGCAAGAGAAAAAAATTTCCCAAACAATGGAATAGAAAGTTTGGTGGATAAAATGAGT
AGATGGAAGACTTATGCTCAGGATCACAGAGACGAGCCTGGGATCATGGGGACTACAAGTAGAGCGAGCC
GTAGACGCCAGCGCCATGACAGACAGAGGGGTCTTGTGTGGGACGATGAGGATTCGGCCGATGATAGCAG
CGTGTTGGACTTGGGTGGGAGAGGAAGGGGCAACCCGTTTGCTCATTTGCGCCCTCGCTTGGGTGGTATG
TTGTGAAAAAAAATAAAAAAGAAAAACTCACCAAGGCCATGGCGACGAGCGTACGTTCGTTCTTCTTTAT
TATCTGTGTCTAGTATAATGAGGCGAGTCGTGCTAGGCGGAGCGGTGGTGTATCCGGAGGGTCCTCCTCC
TTCGTACGAGAGCGTGATGCAGCAGCAGCAGGCGACGGCGGTGATGCAATCCCCACTGGAGGCTCCCTTT
GTGCCTCCGCGATACCTGGCACCTACGGAGGGCAGAAACAGCATTCGTTACTCGGAACTGGCACCTCAGT
ACGATACCACCAGGTTGTATCTGGTGGACAACAAGTCGGCGGACATTGCTTCTCTGAACTATCAGAATGA
CCACAGCAACTTCTTGACCACGGTGGTGCAGAACAATGACTTTACCCCTACGGAAGCCAGCACCCAGACC
ATTAACTTTGATGAACGATCGCGGTGGGGCGGTCAGCTAAAGACCATCATGCATACTAACATGCCAAACG
TGAACGAGTATATGTTTAGTAACAAGTTCAAAGCGCGTGTGATGGTGTCCAGAAAACCTCCCGACGGTGC
TGCAGTTGGGGATACTTATGATCACAAGCAGGATATTTTGGAATATGAGTGGTTCGAGTTTACTTTGCCA
GAAGGCAACTTTTCAGTTACTATGACTATTGATTTGATGAACAATGCCATCATAGATAATTACTTGAAAG
TGGGTAGACAGAATGGAGTGCTTGAAAGTGACATTGGTGTTAAGTTCGACACCAGGAACTTCAAGCTGGG
ATGGGATCCCGAAACCAAGTTGATCATGCCTGGAGTGTATACGTATGAAGCCTTCCATCCTGACATTGTC
TTACTGCCTGGCTGCGGAGTGGATTTTACCGAGAGTCGTTTGAGCAACCTTCTTGGTATCAGAAAAAAAC
AGCCATTTCAAGAGGGTTTTAAGATTTTGTATGAAGATTTAGAAGGTGGTAATATTCCGGCCCTCTTGGA
TGTAGATGCCTATGAGAACAGTAAGAAAGAACAAAAAGCCAAAATAGAAGCTGCTACAGCTGCTGCAGAA
GCTAAGGCAAACATAGTTGCCAGCGACTCTACAAGGGTTGCTAACGCTGGAGAGGTCAGAGGAGACAATT
TTGCGCCAACACCTGTTCCGACTGCAGAATCATTATTGGCCGATGTGTCTGAAGGAACGGACGTGAAACT
CACTATTCAACCTGTAGAAAAAGATAGTAAGAATAGAAGCTATAATGTGTTGGAAGACAAAATCAACACA
GCCTATCGCAGTTGGTATCTTTCGTACAATTATGGCGATCCCGAAAAAGGAGTGCGTTCCTGGACATTGC
TCACCACCTCAGATGTCACCTGCGGAGCAGAGCAGGTTTACTGGTCGCTTCCAGACATGATGAAGGATCC
TGTCACTTTCCGCTCCACTAGACAAGTCAGTAACTACCCTGTGGTGGGTGCAGAGCTTATGCCCGTCTTC
TCAAAGAGCTTCTACAACGAACAAGCTGTGTACTCCCAGCAGCTCCGCCAGTCCACCTCGCTTACGCACG
TCTTCAACCGCTTTCCTGAGAACCAGATTTTAATCCGTCCGCCGGCGCCCACCATTACCACCGTCAGTGA
AAACGTTCCTGCTCTCACAGATCACGGGACCCTGCCGTTGCGCAGCAGTATCCGGGGAGTCCAACGTGTG
ACCGTTACTGACGCCAGACGCCGCACCTGTCCCTACGTGTACAAGGCACTGGGCATAGTCGCACCGCGCG
TCCTTTCAAGCCGCACTTTCTAAAAAAAAAATGTCCATTCTTATCTCGCCCAGTAATAACACCGGTTGGG
GTCTGCGCGCTCCAAGCAAGATGTACGGAGGCGCACGCAAACGTTCTACCCAACATCCCGTGCGTGTTCG
CGGACATTTTCGCGCTCCATGGGGTGCCCTCAAGGGCCGCACTCGCGTTCGAACCACCGTCGATGATGTA
ATCGATCAGGTGGTTGCCGACGCCCGTAATTATACTCCTACTGCGCCTACATCTACTGTGGATGCAGTTA
TTGACAGTGTAGTGGCTGACGCTCGCAACTATGCTCGACGTAAGAGCCGGCGAAGGCGCATTGCCAGACG
CCACCGAGCTACCACTGCCATGCGAGCCGCAAGAGCTCTGCTACGAAGAGCTAGACGCGTGGGGCGAAGA
GCCATGCTTAGGGCGGCCAGACGTGCAGCTTCGGGCGCCAGCGCCGGCAGGTCCCGCAGGCAAGCAGCCG
CTGTCGCAGCGGCGACTATTGCCGACATGGCCCAATCGCGAAGAGGCAATGTATACTGGGTGCGTGACGC
TGCCACCGGTCAACGTGTACCCGTGCGCACCCGTCCCCCTCGCACTTAGAAGATACTGAGCAGTCTCCGA
TGTTGTGTCCCAGCGGCGAGGATGTCCAAGCGCAAATACAAGGAAGAAATGCTGCAGGTTATCGCACCTG
AAGTCTACGGCCAACCGTTGAAGGATGAAAAAAAACCCCGCAAAATCAAGCGGGTTAAAAAGGACAAAAA
AGAAGAGGAAGATGGCGATGATGGGCTGGCGGAGTTTGTGCGCGAGTTTGCCCCACGGCGACGCGTGCAA
TGGCGTGGGCGCAAAGTTCGACATGTGTTGAGACCTGGAACTTCGGTGGTCTTTACACCCGGCGAGCGTT
CAAGCGCTACTTTTAAGCGTTCCTATGATGAGGTGTACGGGGATGATGATATTCTTGAGCAGGCGGCTGA
CCGATTAGGCGAGTTTGCTTATGGCAAGCGTAGTAGAATAACTTCCAAGGATGAGACAGTGTCAATACCC
TTGGATCATGGAAATCCCACCCCTAGTCTTAAACCGGTCACTTTGCAGCAAGTGTTACCCGTAACTCCGC
GAACAGGTGTTAAACGCGAAGGTGAAGATTTGTATCCCACTATGCAACTGATGGTACCCAAACGCCAGAA
GTTGGAGGACGTTTTGGAGAAAGTAAAAGTGGATCCAGATATTCAACCTGAGGTTAAAGTGAGACCCATT
AAGCAGGTAGCGCCTGGTCTGGGGGTACAAACTGTAGACATTAAGATTCCCACTGAAAGTATGGAAGTGC
AAACTGAACCCGCAAAGCCTACTGCCACCTCCACTGAAGTGCAAACGGATCCATGGATGCCCATGCCTAT
TACAACTGACGCCGCCGGTCCCACTCGAAGATCCCGACGAAAGTACGGTCCAGCAAGTCTGTTGATGCCC
AATTATGTTGTACACCCATCTATTATTCCTACTCCTGGTTACCGAGGCACTCGCTACTATCGCAGCCGAA
ACAGTACCTCCCGCCGTCGCCGCAAGACACCTGCAAATCGCAGTCGTCGCCGTAGACGCACAAGCAAACC
GACTCCCGGCGCCCTGGTGCGGCAAGTGTACCGCAATGGTAGTGCGGAACCTTTGACACTGCCGCGTGCG
CGTTACCATCCGAGTATCATCACTTAATCAATGTTGCCGCTGCCTCCTTGCAGATATGGCCCTCACTTGT
CGCCTTCGCGTTCCCATCACTGGTTACCGAGGAAGAAACTCGCGCCGTAGAAGAGGGATGTTGGGACGCG
GAATGCGACGCTACAGGCGACGGCGTGCTATCCGCAAGCAATTGCGGGGTGGTTTTTTACCAGCCTTAAT
TCCAATTATCGCTGCTGCAATTGGCGCGATACCAGGCATAGCTTCCGTGGCGGTTCAGGCCTCGCAACGA
CATTGACATTGGAAAAAAAACGTATAAATAAAAAAAAATACAATGGACTCTGACACTCCTGGTCCTGTGA
CTATGTTTTCTTAGAGATGGAAGACATCAATTTTTCATCCTTGGCTCCGCGACACGGCACGAAGCCGTAC
ATGGGCACCTGGAGCGACATCGGCACGAGCCAACTGAACGGGGGCGCCTTCAATTGGAGCAGTATCTGGA
GCGGGCTTAAAAATTTTGGCTCAACCATAAAAACATACGGGAACAAAGCTTGGAACAGCAGTACAGGACA
GGCGCTTAGAAATAAACTTAAAGACCAGAACTTCCAACAAAAAGTAGTCGATGGGATAGCTTCCGGCATC
AATGGAGTGGTAGATTTGGCTAACCAGGCTGTGCAGAAAAAGATAAACAGTCGTTTGGACCCGCCGCCAG
CAACCCCAGGTGAAATGCAAGTGGAGGAAGAAATTCCTCCGCCAGAAAAACGAGGCGACAAGCGTCCGCG
TCCCGATTTGGAAGAGACGCTGGTGACGCGCGTAGATGAACCGCCTTCTTATGAGGAAGCAACGAAGCTT
GGAATGCCCACCACTAGACCGATAGCCCCAATGGCCACCGGGGTGATGAAACCTTCTCAGTTGCATCGAC
CCGTCACCTTGGATTTGCCCCCTCCCCCTGCTGCTACTGCTGTACCCGCTTCTAAGCCTGTCGCTGCCCC
GAAACCAGTCGCCGTAGCCAGGTCACGTCCCGGGGGCGCTCCTCGTCCAAATGCGCACTGGCAAAATACT
CTGAACAGCATCGTGGGTCTAGGCGTGCAAAGTGTAAAACGCCGTCGCTGCTTTTAATTAAATATGGAGT
AGCGCTTAACTTGCCTATCTGTGTATATGTGTCATTACACGCCGTCACAGCAGCAGAGGAAAAAAGGAAG
AGGTCGTGCGTCGACGCTGAGTTACTTTCAAGATGGCCACCCCATCGATGCTGCCCCAATGGGCATACAT
GCACATCGCCGGACAGGATGCTTCGGAGTACCTGAGTCCGGGTCTGGTGCAGTTCGCCCGCGCCACAGAC
ACCTACTTCAATCTGGGAAATAAGTTTAGAAATCCCACCGTAGCGCCGACCCACGATGTGACCACCGACC
GTAGCCAGCGGCTCATGTTGCGCTTCGTGCCCGTTGACCGGGAGGACAATACATACTCTTACAAAGTGCG
GTACACCCTGGCCGTGGGCGACAACAGAGTGCTGGATATGGCCAGCACGTTCTTTGACATTAGGGGCGTG
TTGGACAGAGGTCCCAGTTTCAAACCCTATTCTGGTACGGCTTACAACTCTCTGGCTCCTAAAGGCGCTC
CAAATGCATCTCAATGGATTGCAAAAGGCGTACCAACTGCAGCAGCCGCAGGCAATGGTGAAGAAGAACA
TGAAACAGAGGAGAAAACTGCTACTTACACTTTTGCCAATGCTCCTGTAAAAGCCGAGGCTCAAATTACA
AAAGAGGGCTTACCAATAGGTTTGGAGATTTCAGCTGAAAACGAATCTAAACCCATCTATGCAGATAAAC
TTTATCAGCCAGAACCTCAAGTGGGAGATGAAACTTGGACTGACCTAGACGGAAAAACCGAAGAGTATGG
AGGCAGGGCTCTAAAGCCTACTACTAACATGAAACCCTGTTACGGGTCCTATGCGAAGCCTACTAATTTA
AAAGGTGGTCAGGCAAAACCGAAAAACTCGGAACCGTCGAGTGAAAAAATTGAATATGATATTGACATGG
AATTTTTTGATAACTCATCGCAAAGAACAAACTTCAGTCCTAAAATTGTCATGTATGCAGAAAATGTAGG
TTTGGAAACGCCAGACACTCATGTAGTGTACAAACCTGGAACAGAAGACACAAGTTCCGAAGCTAATTTG
GGACAACAGTCTATGCCCAACAGACCCAACTACATTGGCTTCAGAGATAACTTTATTGGACTCATGTACT
ATAACAGTACTGGTAACATGGGGGTGCTGGCTGGTCAAGCGTCTCAGTTAAATGCAGTGGTTGACTTGCA
GGACAGAAACACAGAACTTTCTTACCAACTCTTGCTTGACTCTCTGGGCGACAGAACCAGATACTTTAGC
ATGTGGAATCAGGCTGTGGACAGTTATGATCCTGATGTACGTGTTATTGAAAATCATGGTGTGGAAGATG
AACTTCCCAACTATTGTTTTCCACTGGACGGCATAGGTGTTCCAACAACCAGTTACAAATCAATAGTTCC
AAATGGAGAAGATAATAATAATTGGAAAGAACCTGAAGTAAATGGAACAAGTGAGATCGGACAGGGTAAT
TTGTTTGCCATGGAAATTAACCTTCAAGCCAATCTATGGCGAAGTTTCCTTTATTCCAATGTGGCTCTGT
ATCTCCCAGACTCGTACAAATACACCCCGTCCAATGTCACTCTTCCAGAAAACAAAAACACCTACGACTA
CATGAACGGGCGGGTGGTGCCGCCATCTCTAGTAGACACCTATGTGAACATTGGTGCCAGGTGGTCTCTG
GATGCCATGGACAATGTCAACCCATTCAACCACCACCGTAACGCTGGCTTGCGTTACCGATCTATGCTTC
TGGGTAACGGACGTTATGTGCCTTTCCACATACAAGTGCCTCAAAAATTCTTCGCTGTTAAAAACCTGCT
GCTTCTCCCAGGCTCCTACACTTATGAGTGGAACTTTAGGAAGGATGTGAACATGGTTCTACAGAGTTCC
CTCGGTAACGACCTGCGGGTAGATGGCGCCAGCATCAGTTTCACGAGCATCAACCTCTATGCTACTTTTT
TCCCCATGGCTCACAACACCGCTTCCACCCTTGAAGCCATGCTGCGGAATGACACCAATGATCAGTCATT
CAACGACTACCTATCTGCAGCTAACATGCTCTACCCCATTCCTGCCAATGCAACCAATATTCCCATTTCC
ATTCCTTCTCGCAACTGGGCGGCTTTCAGAGGCTGGTCATTTACCAGACTGAAAACCAAAGAAACTCCCT
CTTTGGGGTCTGGATTTGACCCCTACTTTGTCTATTCTGGTTCTATTCCCTACCTGGATGGTACCTTCTA
CCTGAACCACACTTTTAAGAAGGTTTCCATCATGTTTGACTCTTCAGTGAGCTGGCCTGGAAATGACAGG
TTACTATCTCCTAACGAATTTGAAATAAAGCGCACTGTGGATGGCGAAGGCTACAACGTAGCCCAATGCA
ACATGACCAAAGACTGGTTCTTGGTACAGATGCTCGCCAACTACAACATCGGCTATCAGGGCTTCTACAT
TCCAGAAGGATACAAAGATCGCATGTATTCATTTTTCAGAAACTTCCAGCCCATGAGCAGGCAGGTGGTT
GATGAGGTCAATTACAAAGACTTCAAGGCCGTCGCCATACCCTACCAACACAACAACTCTGGCTTTGTGG
GTTACATGGCTCCGACCATGCGCCAAGGTCAACCCTATCCCGCTAACTATCCCTATCCACTCATTGGAAC
AACTGCCGTAAATAGTGTTACGCAGAAAAAGTTCTTGTGTGACAGAACCATGTGGCGCATACCGTTCTCG
AGCAACTTCATGTCTATGGGGGCCCTTACAGACTTGGGACAGAATATGCTCTATGCCAACTCAGCTCATG
CTCTGGACATGACCTTTGAGGTGGATCCCATGGATGAGCCCACCCTGCTTTATCTTCTCTTCGAAGTTTT
CGACGTGGTCAGAGTGCATCAGCCACACCGCGGCATCATCGAGGCAGTCTACCTGCGTACACCGTTCTCG
GCCGGTAACGCTACCACGTAAGAAGCTTCTTGCTTCTTGCAAATAGCAGCTGCAACCATGGCCTGCGGAT
CCCAAAACGGCTCCAGCGAGCAAGAGCTCAGAGCCATTGTCCAAGACCTGGGTTGCGGACCCTATTTTTT
GGGAACCTACGATAAGCGCTTCCCGGGGTTCATGGCCCCCGATAAGCTCGCCTGTGCCATTGTAAATACG
GCCGGACGTGAGACGGGGGGAGAGCACTGGTTGGCTTTCGGTTGGAACCCACGTTCTAACACCTGCTACC
TTTTTGATCCTTTTGGATTCTCGGATGATCGTCTCAAACAGATTTACCAGTTTGAATATGAGGGTCTCCT
GCGCCGCAGCGCTCTTGCTACCAAGGACCGCTGTATTACGCTGGAAAAATCTACCCAGACCGTGCAGGGC
CCCCGTTCTGCCGCCTGCGGACTTTTCTGCTGCATGTTCCTTCACGCCTTTGTGCACTGGCCTGACCGTC
CCATGGACGGAAACCCCACCATGAAATTGCTAACTGGAGTGCCAAACAACATGCTTCATTCTCCTAAAGT
CCAGCCCACCCTGTGTGACAATCAAAAAGCACTCTACCATTTTCTTAATACCCATTCGCCTTATTTTCGC
TCTCATCGTACACACATCGAAAGGGCCACTGCGTTCGACCGTATGGATGTTCAATAATGACTCATGTAAA
CAACGTGTTCAATAAACATCACTTTATTTTTTTACATGTATCAAGGCTCTGGATTACTTATTTATTTACA
AGTCGAATGGGTTCTGACGAGAATCAGAATGACCCGCAGGCAGTGATACGTTGCGGAACTGATACTTGGG
TTGCCACTTGAATTCGGGAATCACCAACTTGGGAACCGGTATATCGGGCAGGATGTCACTCCACAGCTTT
CTGGTCAGCTGCAAAGCTCCAAGCAGGTCAGGAGCCGAAATCTTGAAATCACAATTAGGACCAGTGCTCT
GAGCGCGAGAGTTGCGGTACACCGGATTGCAGCACTGAAACACCATCAGCGACGGATGTCTCACGCTTGC
CAGCACGGTGGGATCTGCAATCATGCCCACATCCAGATCTTCAGCATTGGCAATGCTGAACGGGGTCATC
TTGCAGGTCTGCCTACCCATGGCGGGCACCCAATTAGGCTTGTGGTTGCAATCGCAGTGCAGGGGGATCA
GTATCATCTTGGCCTGATCCTGTCTGATTCCTGGATACACGGCTCTCATGAAAGCATCATATTGCTTGAA
AGCCTGCTGGGCTTTACTACCCTCGGTATAAAACATCCCGCAGGACCTGCTCGAAAACTGGTTAGCTGCA
CAGCCGGCATCATTCACACAGCAGCGGGCGTCATTGTTGGCTATTTGCACCACACTTCTGCCCCAGCGGT
TTTGGGTGATTTTGGTTCGCTCGGGATTCTCCTTTAAGGCTCGTTGTCCGTTCTCGCTGGCCACATCCAT
CTCGATAATCTGCTCCTTCTGAATCATAATATTGCCATGCAGGCACTTCAGCTTGCCCTCATAATCATTG
CAGCCATGAGGCCACAACGCACAGCCTGTACATTCCCAATTATGGTGGGCGATCTGAGAAAAAGAATGTA
TCATTCCCTGCAGAAATCTTCCCATCATCGTGCTCAGTGTCTTGTGACTAGTGAAAGTTAACTGGATGCC
TCGGTGCTCTTCGTTTACGTACTGGTGACAGATGCGCTTGTATTGTTCGTGTTGCTCAGGCATTAGTTTA
AAACAGGTTCTAAGTTCGTTATCCAGCCTGTACTTCTCCATCAGCAGACACATCACTTCCATGCCTTTCT
CCCAAGCAGACACCAGGGGCAAGCTAATCGGATTCTTAACAGTGCAGGCAGCAGCTCCTTTAGCCAGAGG
GTCATCTTTAGCGATCTTCTCAATGCTTCTTTTGCCATCCTTCTCAACGATGCGCACGGGCGGGTAGCTG
AAACCCACTGCTACAAGTTGCGCCTCTTCTCTTTCTTCTTCGCTGTCTTGACTGATGTCTTGCATGGGGA
TATGTTTGGTCTTCCTTGGCTTCTTTTTGGGGGGTATCGGAGGAGGAGGACTGTCGCTCCGTTCCGGAGA
CAGGGAGGATTGTGACGTTTCGCTCACCATTACCAACTGACTGTCGGTAGAAGAACCTGACCCCACACGG
CGACAGGTGTTTTTCTTCGGGGGCAGAGGTGGAGGCGATTGCGAAGGGCTGCGGTCCGACCTGGAAGGCG
GATGACTGGCAGAACCCCTTCCGCGTTCGGGGGTGTGCTCCCTGTGGCGGTCGCTTAACTGATTTCCTTC
GCGGCTGGCCATTGTGTTCTCCTAGGCAGAGAAACAACAGACATGGAAACTCAGCCATTGCTGTCAACAT
CGCCACGAGTGCCATCACATCTCGTCCTCAGCGACGAGGAAAAGGAGCAGAGCTTAAGCATTCCACCGCC
CAGTCCTGCCACCACCTCTACCCTAGAAGATAAGGAGGTCGACGCATCTCATGACATGCAGAATAAAAAA
GCGAAAGAGTCTGAGACAGACATCGAGCAAGACCCGGGCTATGTGACACCGGTGGAACACGAGGAAGAGT
TGAAACGCTTTCTAGAGAGAGAGGATGAAAACTGCCCAAAACAGCGAGCAGATAACTATCACCAAGATGC
TGGAAATAGGGATCAGAACACCGACTACCTCATAGGGCTTGACGGGGAAGACGCGCTCCTTAAACATCTA
GCAAGACAGTCGCTCATAGTCAAGGATGCATTATTGGACAGAACTGAAGTGCCCATCAGTGTGGAAGAGC
TCAGCTGCGCCTACGAGCTTAACCTTTTTTCACCTCGTACTCCCCCCAAACGTCAGCCAAACGGCACCTG
CGAGCCAAATCCTCGCTTAAACTTTTATCCAGCTTTTGCTGTGCCAGAAGTACTGGCTACCTATCACATC
TTTTTTAAAAATCAAAAAATTCCAGTCTCCTGCCGCGCTAATCGCACCCGCGCCGATGCCCTACTCAATC
TGGGACCTGGTTCACGCTTACCTGATATAGCTTCCTTGGAAGAGGTTCCAAAGATCTTCGAGGGTCTGGG
CAATAATGAGACTCGGGCCGCAAATGCTCTGCAAAAGGGAGAAAATGGCATGGATGAGCATCACAGCGTT
CTGGTGGAATTGGAAGGCGATAATGCCAGACTCGCAGTACTCAAGCGAAGCGTCGAGGTCACACACTTCG
CATATCCCGCTGTCAACCTGCCCCCTAAAGTCATGACGGCGGTCATGGACCAGTTACTCATTAAGCGCGC
AAGTCCCCTTTCAGAAGACATGCATGACCCAGATGCCTGTGATGAGGGTAAACCAGTGGTCAGTGATGAG
CAGCTAACCCGATGGCTGGGCACCGACTCTCCCCGGGATTTGGAAGAGCGTCGCAAGCTTATGATGGCCG
TGGTGCTGGTTACCGTAGAACTAGAGTGTCTCCGACGTTTCTTTACCGATTCAGAAACCTTGCGCAAACT
CGAAGAGAATCTGCACTACACTTTTAGACACGGCTTTGTGCGGCAGGCATGCAAGATATCTAACGTGGAA
CTCACCAACCTGGTTTCCTACATGGGTATTCTGCATGAGAATCGCCTAGGACAAAGCGTGCTGCACAGCA
CCCTTAAGGGGGAAGCCCGCCGTGATTACATCCGCGATTGTGTCTATCTCTACCTGTGCCACACGTGGCA
AACCGGCATGGGTGTATGGCAGCAATGTTTAGAAGAACAGAACTTGAAAGAGCTTGACAAGCTCTTACAG
AAATCTCTTAAGGTTCTGTGGACAGGGTTCGACGAGCGCACCGTCGCTTCCGACCTGGCAGACCTCATCT
TCCCAGAGCGTCTCAGGGTTACTTTGCGAAACGGATTGCCTGACTTTATGAGCCAGAGCATGCTTAACAA
TTTTCGCTCTTTCATCCTGGAACGCTCCGGTATCCTGCCCGCCACCTGCTGCGCACTGCCCTCCGACTTT
GTGCCTCTCACCTACCGCGAGTGCCCCCCGCCGCTATGGAGTCACTGCTACCTGTTCCGTCTGGCCAACT
ATCTCTCCTACCACTCGGATGTGATCGAGGATGTGAGCGGAGACGGCTTGCTGGAGTGCCACTGCCGCTG
CAATCTGTGCACGCCCCACCGGTCCCTAGCTTGCAACCCCCAGTTGATGAGCGAAACCCAGATAATAGGC
ACCTTTGAATTGCAAGGCCCCAGCAGCCAAGGCGATGGGTCTTCTCCTGGGCAAAGTTTAAAACTGACCC
CGGGACTGTGGACCTCCGCCTACTTGCGCAAGTTTGCTCCGGAAGATTACCACCCCTATGAAATCAAGTT
CTATGAGGACCAATCACAGCCTCCAAAGGCCGAACTTTCGGCTTGCGTCATCACCCAGGGGGCAATTCTG
GCCCAATTGCAAGCCATCCAAAAATCCCGCCAAGAATTTCTACTGAAAAAGGGTAAGGGGGTCTACCTTG
ACCCCCAGACCGGCGAGGAACTCAACACAAGGTTCCCTCAGGATGTCCCAACGACGAGAAAACAAGAAGT
TGAAGGTGCAGCCGCCGCCCCCAGAAGATATGGAGGAAGATTGGGACAGTCAGGCAGAGGAGGCGGAGGA
GGACAGTCTGGAGGACAGTCTGGAGGAAGACAGTTTGGAGGAGGAAAACGAGGAGGCAGAGGAGGTGGAA
GAAGTAACCGCCGACAAACAGTTATCCTCGGCTGCGGAGACAAGCAACAGCGCTACCATCTCCGCTCCGA
GTCGAGGAACCCGGCGGCGTCCCAGCAGTAGATGGGACGAGACCGGACGCTTCCCGAACCCAACCAGCGC
TTCCAAGACCGGTAAGAAGGATCGGCAGGGATACAAGTCCTGGCGGGGGCATAAGAATGCCATCATCTCC
TGCTTGCATGAGTGCGGGGGCAACATATCCTTCACGCGGCGCTACTTGCTATTCCACCATGGGGTGAACT
TTCCGCGCAATGTTTTGCATTACTACCGTCACCTCCACAGCCCCTACTATAGCCAGCAAATCCCGACAGT
CTCGACAGATAAAGACAGCGGCGGCGACCTCCAACAGAAAACCAGCAGCGGCAGTTAGAAAATACACAAC
AAGTGCAGCAACAGGAGGATTAAAGATTACAGCCAACGAGCCAGCGCAAACCCGAGAGTTAAGAAATCGG
ATCTTTCCAACCCTGTATGCCATCTTCCAGCAGAGTCGGGGTCAAGAGCAGGAACTGAAAATAAAAAACC
GATCTCTGCGTTCGCTCACCAGAAGTTGTTTGTATCACAAGAGCGAAGATCAACTTCAGCGCACTCTCGA
GGACGCCGAGGCTCTCTTCAACAAGTACTGCGCGCTGACTCTTAAAGAGTAGGCAGCGACCGCGCTTATT
CAAAAAAGGCGGGAATTACATCATCCTCGACATGAGTAAAGAAATTCCCACGCCTTACATGTGGAGTTAT
CAACCCCAAATGGGATTGGCAGCAGGCGCCTCCCAGGACTACTCCACCCGCATGAATTGGCTCAGCGCCG
GGCCTTCTATGATTTCTCGAGTTAATGATATACGCGCCTACCGAAACCAAATACTTTTGGAACAGTCAGC
TCTTACCACCACGCCCCGCCAACACCTTAATCCCAGAAATTGGCCCGCCGCCCTAGTGTACCAGGAAAGT
CCCGCTCCCACCACTGTATTACTTCCTCGAGACGCCCAGGCCGAAGTCCAAATGACTAATGCAGGTGCGC
AGTTAGCTGGCGGCTCCACCCTATGTCGTCACAGGCCTCGGCATAATATAAAACGCCTGATGATCAGAGG
CCGAGGTATCCAGCTCAACGACGAGTCGGTGAGCTCTCCGCTTGGTCTACGACCAGACGGAATCTTTCAG
ATTGCCGGCTGCGGGAGATCTTCCTTCACCCCTCGTCAGGCTGTTCTGACTTTGGAAAGTTCGTCTTCGC
AACCCCGCTCGGGCGGAATCGGGACCGTTCAATTTGTAGAGGAGTTTACTCCCTCTGTCTACTTCAACCC
CTTCTCCGGATCTCCTGGGCACTACCCGGACGAGTTCATACCGAACTTCGACGCGATTAGCGAGTCAGTG
GACGGCTACGATTGATGTCTGGTGACGCGGCTGAGCTATCTCGGCTGCGACATCTAGACCACTGCCGCCG
CTTTCGCTGCTTTGCCCGGGAACTTATTGAGTTCATCTACTTCGAACTCCCCAAGGATCACCCTCAAGGT
CCGGCCCACGGAGTGCGGATTACTATCGAAGGCAAAATAGACTCTCGCCTGCAACGAATTTTCTCCCAGC
GGCCCGTGCTGATCGAGCGAGACCAGGGAAACACCACGGTTAGTAATCAATTACGGGGTCATTAGTTCAT
AGCCCATATATGGAGTTAACTTGTTTATTGCAGCTTATAATGGTTACAAATAAAGCAATAGCATCACAAA
TTTCACAAATAAAGCATTTTTTTCACTGCATTCTAGTTGTGGTTTGTCCAAACTCATCAATGTATCTTAT
CATGTCTGCTCGAAGCGGCCGGCCGCCCCGACTCTAGAGTCGCGGCCTCATTAGGAAGTTCCTATACTTT
CTAGAGAATAGGAACTTCTCAGAAGAACTCGTCAAGAAGGCGATAGAAGGCGATGCGCTGCGAATCGGGA
GCGGCGATACCGTAAAGCACGAGGAAGCGGTCAGCCCATTCGCCGCCAAGCTCTTCAGCAATATCACGGG
TAGCCAACGCTATGTCCTGATAGCGGTCCGCCACACCCAGCCGGCCACAGTCGATGAATCCAGAAAAGCG
GCCATTTTCCACCATGATATTCGGCAAGCAGGCATCGCCATGGGTCACGACGAGATCCTCGCCGTCGGGC
ATGCGCGCCTTGAGCCTGGCGAACAGTTCGGCTGGCGCGAGCCCCTGATGCTCTTCGTCCAGATCATCCT
GATCGACAAGACCGGCTTCCATCCGAGTACGTGCTCGCTCGATGCGATGTTTCGCTTGGTGGTCGAATGG
GCAGGTAGCCGGATCAAGCGTATGCAGCCGCCGCATTGCATCAGCCATGATGGATACTTTCTCGGCAGGA
GCAAGGTGAGATGACAGGAGATCCTGCCCCGGCACTTCGCCCAATAGCAGCCAGTCCCTTCCCGCTTCAG
TGACAACGTCGAGCACAGCTGCGCAAGGAACGCCCGTCGTGGCCAGCCACGATAGCCGCGCTGCCTCGTC
CTGCAGTTCATTCAGGGCACCGGACAGGTCGGTCTTGACAAAAAGAACCGGGCGCCCCTGCGCTGACAGC
CGGAACACGGCGGCATCAGAGCAGCCGATTGCCTGTTGTGCCCAGTCATAGCCGAATAGCCTCTCCACCC
AAGCGGCCGGAGAACCTGCGTGCAATCCATCTTGTTCAATGGCCGATCCCATAACACCCCTTGTATTACT
GTTTATGTAAGCAGACAGTTTTACTGTTCGTGATGATATATTTTTATCTTGTGCAATGTAACAGGTTGTG
GCCATAGCGGGCCCGGGATTTTCCTCCACGTCCCCGCATGTTAGAAGACTTCCCCTGCCCTCGGCTCTGG
AAGTTCCTATACTTTCTAGAGAATAGGAACTTCCCGCCAGAATGCGTTCGCACAGCCGCCAGCCGGTCAC
TCCGTTGATGGTTACTCGGAACAGCAGGGAGCCGTCGGGGTTGATCAGGCGCTCGTCGATAATTTTGTTG
CCGTTCCACAGGGTCCCTGTTACAGTGATCTTTTTGCCGTCGAACACGGCGATGCCTTCATACGGCCGTC
CGAAATAGTCGATCATGTTCGGCGTAACCCCGTCGATTACCAGTGTGCCATAGTGCAGGATCACCTTAAA
GTGATGATCATCCACAGGGTACACCACCTTAAAAATTTTTTCGATCTGGCCCATTTGGTCGCCGCTCAGA
CCTTCATACGGGATGATGACATGGATGTCGATCTTCAGCCCATTTTCACCGCTCAGGACAATCCTTTGGA
TCGGAGTTACGGACACCCCGAGATTCTGAAACAAACTGGACACACCTCCCTGTTCAAGGACTTGGTCCAG
GTTGTAGCCGGCTGTCTGTCGCCAGTCCCCAACGAAATCTTCGAGTGTGAAGACCATGGATCCGGGCCCG
GGGTTTTCTTCAACGTCTCCAGCCTGCTTCAGCAGGCTGAAGTTAGTAGCTCCGCTTCCTCGAGCTCGAG
ATCTGGCGAAGGCGATGGGGGTCTTGAAGGCGTGCTGGTACTCCACGATGCCCAGCTCGGTGTTGCTGTG
CAGCTCCTCCACGCGGCGGAAGGCGAACATGGGGCCCCCGTTCTGCAGGATGCTGGGGTGGATGGCGCTC
TTGAAGTGCATGTGGCTGTCCACCACGAAGCTGTAGTAGCCGCCGTCGCGCAGGCTGAAGGTGCGGGCGA
AGCTGCCCACCAGCACGTTATCGCCCATGGGGTGCAGGTGCTCCACGGTGGCGTTGCTGCGGATGATCTT
GTCGGTGAAGATCACGCTGTCCTCGGGGAAGCCGGTGCCCACCACCTTGAAGTCGCCGATCACGCGGCCG
GCCTCGTAGCGGTAGCTGAAGCTCACGTGCAGCACGCCGCCGTCCTCGTACTTCTCGATGCGGGTGTTGG
TGTAGCCGCCGTTGTTGATGGCGTGCAGGAAGGGGTTCTCGTAGCCGCTGGGGTAGGTGCCGAAGTGGTA
GAAGCCGTAGCCCATCACGTGGCTCAGCAGGTAGGGGCTGAAGGTCAGGGCGCCTTTGGTGCTCTTCATC
TTGTTGGTCATGCGGCCCTGCTCGGGGGTGCCCTCTCCGCCGCCCACCAGCTCGAACTCCACGCCGTTCA
GGGTGCCGGTGATGCGGCACTCGATCTTCATGGCGGGCATGGTGGCGACCGGTAGCGCTAGCGGCTTCGG
TACCACGCGTTCGCTCGAATTAATCAATTCTTTGCCAAAATGATGAGACAGCACAATAACCAGCACGTTG
CCCAGGAGCTGTAGGAAAAAGAAGAAGGCATGAACATGGTTAGCAGAGGCTCTAGAGCCGCCGGTCACAC
GCCAGAAGCCGAACCCCGCCCTGCCCCGTCCCCCCCGAAGGCAGCCGTCCCCCCGCGGACAGCCCCGAGG
CTGGAGAGGGAGAAGGGGACGGCGGCGCGGCGACGCACGAAGGCCCTCCCCGCCCATTTCCTTCCTGCCG
GGGCCCTCCCGGAGCCCCTCAAGGCTTTCACGCAGCCACAGAAAAGAAACAAGCCGTCATTAAACCAAGC
GCTAATTACAGCCCGGAGGAGAAGGGCCGTCCCGCCCGCTCACCTGTGGGAGTAACGCGGTCAGTCAGAG
CCGGGGCGGGCGGCGCGAGGCGGCGCGGAGCGGGGCACGGGGCGAAGGCAACGCAGCGACTCCCGCCCGC
CGCGCGCTTCGCTTTTTATAGGGCCGCCGCCGCCGCCGCCTCGCCATAAAAGGAAACTTTCGGAGCGCGC
CGCTCTGATTGGCTGCCGCCGCACCTCTCCGCCTCGCCCCGCCCCGCCCCTCGCCCCGCCCCGCCCCGCC
TGGCGCGCGCCCCCCCCCCCCCCCCGCCCCCATCGCTGCACAAAATAATTAAAAAATAAATAAATACAAA
ATTGGGGGTGGGGAGGGGGGGGAGATGGGGAGAGTGAAGCAGAACGTGGGGCTCACCTCGACCATGGTAA
TAGCGATGACTAATACGTAGATGTACTGCCAAGTAGGAAAGTCCCATAAGGTCATGTACTGGGCATAATG
CCAGGCGGGCCATTTACCGTCATTGACGTCAATAGGGGGCGTACTTGGCATATGATACACTTGATGTACT
GCCAAGTGGGCAGTTTACCGTAAATACTCCACCCATTGACGTCAATGGAAAGTCCCTATTGGCGTTACTA
TGGGAACATACGTCATTATTGACGTCAATGGGCGGGGGTCGTTGGGCGGTCAGCCAGGCGGGCCATTTAC
CGTAAGTTATGTAACGCGGAACAACAACAATTGCATTCATTTTATGTTTCAGGTTCAGGGGGAGGTGTGG
TCCTGCGATTCCATCGAGTGCACCTACACCCTGCTGAAGACCCTATGCGGCCTAAGAGACCTGCTACCAA
TGAATTAAAAAAAAATGATTAATAAAAAATCACTTACTTGAAATCAGCAATAAGGTCTCTGTTGAAATTT
TCTCCCAGCAGCACCTCACTTCCCTCTTCCCAACTCTGGTATTCTAAACCCCGTTCAGCGGCATACTTTC
TCCATACTTTAAAGGGGATGTCAAATTTTAGCTCCTCTCCTGTACCCACAATCTTCATGTCTTTCTTCCC
AGATGACCAAGAGAGTCCGGCTCAGTGACTCCTTCAACCCTGTCTACCCCTATGAAGATGAAAGCACCTC
CCAACACCCCTTTATAAACCCAGGGTTTATTTCCCCAAATGGCTTCACACAAAGCCCAGACGGAGTTCTT
ACTTTAAAATGTTTAACCCCACTAACAACCACAGGCGGATCTCTACAGCTAAAAGTGGGAGGGGGACTTA
CAGTGGATGACACTGATGGTACCTTACAAGAAAACATACGTGCTACAGCACCCATTACTAAAAATAATCA
CTCTGTAGAACTATCCATTGGAAATGGATTAGAAACTCAAAACAATAAACTATGTGCCAAATTGGGAAAT
GGGTTAAAATTTAACAACGGTGACATTTGTATAAAGGATAGTATTAACACCTTATGGACTGGAATAAACC
CTCCACCTAACTGTCAAATTGTGGAAAACACTAATACAAATGATGGCAAACTTACTTTAGTATTAGTAAA
AAATGGAGGGCTTGTTAATGGCTACGTGTCTCTAGTTGGTGTATCAGACACTGTGAACCAAATGTTCACA
CAAAAGACAGCAAACATCCAATTAAGATTATATTTTGACTCTTCTGGAAATCTATTAACTGAGGAATCAG
ACTTAAAAATTCCACTTAAAAATAAATCTTCTACAGCGACCAGTGAAACTGTAGCCAGCAGCAAAGCCTT
TATGCCAAGTACTACAGCTTATCCCTTCAACACCACTACTAGGGATAGTGAAAACTACATTCATGGAATA
TGTTACTACATGACTAGTTATGATAGAAGTCTATTTCCCTTGAACATTTCTATAATGCTAAACAGCCGTA
TGATTTCTTCCAATGTTGCCTATGCCATACAATTTGAATGGAATCTAAATGCAAGTGAATCTCCAGAAAG
CAACATAGCTACGCTGACCACATCCCCCTTTTTCTTTTCTTACATTACAGAAGACGACAACTAAAATAAA
GTTTAAGTGTTTTTATTTAAAATCACAAAATTCGAGTAGTTATTTTGCCTCCACCTTCCCATTTGACAGA
ATACACAGTCCTTTCTCCCCGGCTGGCCTTAAAAAGCATCATATCATGGGTAACAGACATATTCTTAGGT
GTTATATTCCACACGGTTTCCTGTCGAGCCAAACGCTCATCAGTGATATTAATAAACTCCCCGGGCAGCT
CACTTAAGTTCATGTCGCTGTCCAGCTGCTGAGCCACAGGCTGCTGTCCAACTTGCGGTTGCTTAACGGG
CGGCGAAGGAGAAGTCCACGCCTACATGGGGGTAGAGTCATAATCGTGCATCAGGATAGGGCGGTGGTGC
TGCAGCAGCGCGCGAATAAACTGCTGCCGCCGCCGCTCCGTCCTGCAGGAATACAACATGGCAGTGGTCT
CCTCAGCGATGATTCGCACCGCCCGCAGCATAAGGCGCCTTGTCCTCCGGGCACAGCAGCGCACCCTGAT
CTCACTTAAATCAGCACAGTAACTGCAGCACAGCACCACAATATTGTTCAAAATCCCACAGTGCAAGGCG
CTGTATCCAAAGCTCATGGCGGGGACCACAGAACCCACGTGGCCATCATACCACAAGCGCAGGTAGATTA
AGTGGCGACCCCTCATAAACACGCTGGACATAAACATTACCTCTTTTGGCATGTTGTAATTCACCACCTC
CCGGTACCATATAAACCTCTGATTAAACATGGCGCCATCCACCACCATCCTAAACCAGCTGGCCAAAACC
TGCCCGCCGGCTATACACTGCAGGGAACCGGGACTGGAACAATGACAGTGGAGAGCCCAGGACTCGTAAC
CATGGATCATCATGCTCGTCATGATATCAATGTTGGCACAACACAGGCACACGTGCATACACTTCCTCAG
GATTACAAGCTCCTCCCGCGTTAGAACCATATCCCAGGGAACAACCCATTCCTGAATCAGCGTAAATCCC
ACACTGCAGGGAAGACCTCGCACGTAACTCACGTTGTGCATTGTCAAAGTGTTACATTCGGGCAGCAGCG
GATGATCCTCCAGTATGGTAGCGCGGGTTTCTGTCTCAAAAGGAGGTAGACGATCCCTACTGTACGGAGT
GCGCCGAGACAACCGAGATCGTGTTGGTCGTAGTGTCATGCCAAATGGAACGCCGGACGTAGTCATTCTC
GTATTTTGTATAGCAAAACGCGGCCCTGGCAGAACACACTCTTCTTCGCCTTCTATCCTGCCGCTTAGCG
TGTTCCGTGTGATAGTTCAAGTACAGCCACACTCTTAAGTTGGTCAAAAGAATGCTGGCTTCAGTTGTAA
TCAAAACTCCATCGCATCTAATTGTTCTGAGGAAATCATCCACGGTAGCATATGCAAATCCCAACCAAGC
AATGCAACTGGATTGCGTTTCAAGCAGGAGAGGAGAGGGAAGAGACGGAAGAACCATGTTAATTTTTATT
CCAAACGATCTCGCAGTACTTCAAATTGTAGATCGCGCAGATGGCATCTCTCGCCCCCACTGTGTTGGTG
AAAAAGCACAGCTAAATCAAAAGAAATGCGATTTTCAAGGTGCTCAACGGTGGCTTCCAACAAAGCCTCC
ACGCGCACATCCAAGAACAAAAGAATACCAAAAGAAGGAGCATTTTCTAACTCCTCAATCATCATATTAC
ATTCCTGCACCATTCCCAGATAATTTTCAGCTTTCCAGCCTTGAATTATTCGTGTCAGTTCTTGTGGTAA
ATCCAATCCACACATTACAAACAGGTCCCGGAGGGCGCCCTCCACCACCATTCTTAAACACACCCTCATA
ATGACAAAATATCTTGCTCCTGTGTCACCTGTAGCGAATTGAGAATGGCAACATCAATTGACATGCCCTT
GGCTCTAAGTTCTTCTTTAAGTTCTAGTTGTAAAAACTCTCTCATATTATCACCAAACTGCTTAGCCAGA
AGCCCCCCGGGAACAAGAGCAGGGGACGCTACAGTGCAGTACAAGCGCAGACCTCCCCAATTGGCTCCAG
CAAAAACAAGATTGGAATAAGCATATTGGGAACCACCAGTAATATCATCGAAGTTGCTGGAAATATAATC
AGGCAGAGTTTCTTGTAGAAATTGAATAAAAGAAAAATTTGCCAAAAAAACATTCAAAACCTCTGGGATG
CAAATGCAATAGGTTACCGCGCTGCGCTCCAACATTGTTAGTTTTGAATTAGTCTGCAAAAATAAAAAAA
AAACAAGCGTCATATCATAGTAGCCTGACGAACAGGTGGATAAATCAGTCTTTCCATCACAAGACAAGCC
ACAGGGTCTCCAGCTCGACCCTCGTAAAACCTGTCATCGTGATTAAACAACAGCACCGAAAGTTCCTCGC
GGTGACCAGCATGAATAAGTCTTGATGAAGCATACAATCCAGACATGTTAGCATCAGTTAAGGAGAAAAA
ACAGCCAACATAGCCTTTGGGTATAATTATGCTTAATCGTAAGTATAGCAAAGCCACCCCTCGCGGATAC
AAAGTAAAAGGCACAGGAGAATAAAAAATATAATTATTTCTCTGCTGCTGTTTAGGCAACGTCGCCCCCG
GTCCCTCTAAATACACATACAAAGCCTCATCAGCCATGGCTTACCAGAGAAAGTACAGCGGGCACACAAA
CCACAAGCTCTAAAGTCACTCTCCAACCTCTCCACAATATATATACACAAGCCCTAAACTGACGTAATGG
GACTAAAGTGTAAAAAATCCCGCCAAACCCAACACACACCCCGAAACTGCGTCACCAGGGAAAAGTACAG
TTTCACTTCCGCAATCCCAACAAGCGTCACTTCCTCTTTCTCACGGTACGTCACATCCCATTAACTTACA
ACGTCATTTTCCCACGGCCGCGCCGCCCCTTTTAACCGTTAACCCCACAGCCAATCACCACACGGCCCAC
ACTTTTTAAAATCACCTCATTTACATATTGGCACCATTCCATCTATAAGGTATATTATTGATGATGGCCA
AGCTATTTAGGTGACACTATAGAATACTCAAGCTATGCATCAAGCTTGGTACCGAGCTCGGATCCACTAG
TAACGGCCGCCAGTGTGCTGGAATTCGCCCTTGTTTAAACGCGATCGCTTGAGATCGTTTTGGTCTGCGC
GTAATCTCTTGCTCTGAAAACGAAAAAACCGCCTTGCAGGGCGGTTTTTCGAAGGTTCTCTGAGCTACCA
ACTCTTTGAACCGAGGTAACTGGCTTGGAGGAGCGCAGTCACCAAAACTTGTCCTTTCAGTTTAGCCTTA
ACCGGCGCATGACTTCAAGACTAACTCCTCTAAATCAATTACCAGTGGCTGCTGCCAGTGGTGCTTTTGC
ATGTCTTTCCGGGTTGGACTCAAGACGATAGTTACCGGATAAGGCGCAGCGGTCGGACTGAACGGGGGGT
TCGTGCATACAGTCCAGCTTGGAGCGAACTGCCTACCCGGAACTGAGTGTCAGGCGTGGAATGAGACAAA
CGCGGCCATAACAGCGGAATGACACCGGTAAACCGAAAGGCAGGAACAGGAGAGCGCACGAGGGAGCCGC
CAGGGGGAAACGCCTGGTATCTTTATAGTCCTGTCGGGTTTCGCCACCACTGATTTGAGCGTCAGATTTC
GTGATGCTTGTCAGGGGGGCGGAGCCTATGGAAAAACGGCTTTGCCGCGGCCCTCTCACTTCCCTGTTAA
GTATCTTCCTGGCATCTTCCAGGAAATCTCCGCCCCGTTCGTAAGCCATTTCCGCTCGCCGCAGTCGAAC
GACCGAGCGTAGCGAGTCAGTGAGCGAGGAAGCGGAATATATCCTGTATCACATATTCTGCTGACGCACC
GGTGCAGCCTTTTTTCTCCTGCCACATGAAGCACTTCACTGACACCCTCATCAGTGCCAACATAGTAAGC
CAGTATACACTCCGCTAGCGCGATCGCTTAATTAATTTAAATCCTGCAGGGTTTAAACGGCCGGCCTAGG
GATAACAGGGTAATCGTAACTATAACGGTCCTAAGGTAGCGAATGATGTCCGGCGGTGCTTTTGCCGTTA
CGCACCACCCCGTCAGTAGCTGAACAGGAGGGACAGCTGATAGAAACAGAAGCCACTGGAGCACCTCAAA
AACACCATCATACACTAAATCAGTAAGTTGGCAGCATCACCCGACGCACTTTGCGCCGAATAAATACCTG
TGACGGAAGATCACTTCGCAGAATAAATAAATCCTGGTGTCCCTGTTGATACCGGGAAGCCCTGGGCCAA
CTTTTGGCGAAAATGAGACGTTGATCGGCACGTAAGAGGTTCCAACTTTCACCATAATGAAATAAGATCA
CTACCGGGCGTATTTTTTGAGTTATCGAGATTTTCAGGAGCTAAGGAAGCTAAAATGGAGAAAAAAATCA
CTGGATATACCACCGTTGATATATCCCAATGGCATCGTAAAGAACATTTTGAGGCATTTCAGTCAGTTGC
TCAATGTACCTATAACCAGACCGTTCAGCTGGATATTACGGCCTTTTTAAAGACCGTAAAGAAAAATAAG
CACAAGTTTTATCCGGCCTTTATTCACATTCTTGCCCGCCTGATGAATGCTCATCCGGAATTCCGTATGG
CAATGAAAGACGGTGAGCTGGTGATATGGGATAGTGTTCACCCTTGTTACACCGTTTTCCATGAGCAAAC
TGAAACGTTTTCATCGCTCTGGAGTGAATACCACGACGATTTCCGGCAGTTTCTACACATATATTCGCAA
GATGTGGCGTGTTACGGTGAAAACCTGGCCTATTTCCCTAAAGGGTTTATTGAGAATATGTTTTTCGTCT
CAGCCAATCCCTGGGTGAGTTTCACCAGTTTTGATTTAAACGTGGCCAATATGGACAACTTCTTCGCCCC
CGTTTTCACCATGGGCAAATATTATACGCAAGGCGACAAGGTGCTGATGCCGCTGGCGATTCAGGTTCAT
CATGCCGTCTGTGATGGCTTCCATGTCGGCAGAATGCTTAATGAATTACAACAGTACTGCGATGAGTGGC
AGGGCGGGGCGTAACCTGCAGGTTAATTAAGGAAGGGCGAATTCTGCAGATATCCATCACACTGGCGGCC
GCTCGAGCATGCATCTAGAGGGCCCAATTCGCCCTATAGTGAGTCGTATTACAATTCACTGGCC (SEQ
ID NO: 176)
The nucleotide sequence of PS3:
CATCATCAATAATATACCTTATAGATGGAATGGTGCCAATATGTAAATGAGGTGATTTTAAAAAGTGTGG
GCCGTGTGGTGATTGGCTGTGGGGTTAACGGTTAAAAGGGGCGGCGCGGCCGTGGGAAAATGACGTTTTA
TGGGGGTGGAGTTTATAACTTCGTATAGCATACATTATACGAAGTTATTTTTGCAAGTTGTCGCGGGAAA
TGTTACGCATAAAAAGGCTTCTTTTCTCACGGAACTACTTAGTTTTCCCACGGTATTTAACAGGAAATGA
GGTAGTTTTGACCGGATGCAAGTGAAAATTGCTGATTTTCGCGCGAAAACTGAATGAGGAAGTGTTTTTC
TGAATAATGTGGTATTTATGGCAGGGTGGAGTATTTGTTCAGGGCCAGGTAGACTTTGACCCATTACGTG
GAGGTTTCGATTACCGTGTTTTTTACCTGAATTTCCGCGTACCGTGTCAAAGTCTTCTGTTTTTACGTAG
GTGTCAGCTGATCGCTAGGGTATTTATAACTTCGTATAGCATACATTATACGAAGTTATGGAATGTTTAT
GCCTTACCAGTGTAACATGAATCATGTGAAAGTGTTGTTGGAACCAGATGCCTTTTCCAGAATGAGCCTA
ACAGGAATCTTTGACATGAACACGCAAATCTGGAAGATCCTGAGGTATGATGATACGAGATCGAGGGTGC
GCGCATGCGAATGCGGAGGCAAGCATGCCAGGTTCCAGCCGGTGTGTGTAGATGTGACCGAAGATCTCAG
ACCGGATCATTTGGTTATTGCCCGCACTGGAGCAGAGTTCGGATCCAGTGGAGAAGAAACTGACTAAGGT
GAGTATTGGGAAAACTTTGGGGTGGGATTTTCAGATGGACAGATTGAGTAAAAATTTGTTTTTTCTGTCT
TGCAGCTGACATGAGTGGAAATGCTTCTTTTAAGGGGGGAGTCTTCAGCCCTTATCTGACAGGGCGTCTC
CCATCCTGGGCAGGAGTTCGTCAGAATGTTATGGGATCTACTGTGGATGGAAGACCCGTTCAACCCGCCA
ATTCTTCAACGCTGACCTATGCTACTTTAAGTTCTTCACCTTTGGACGCAGCTGCAGCCGCTGCCGCCGC
CTCTGTCGCCGCTAACACTGTGCTTGGAATGGGTTACTATGGAAGCATCGTGGCTAATTCCACTTCCTCT
AATAACCCTTCTACACTGACTCAGGACAAGTTACTTGTCCTTTTGGCCCAGCTGGAGGCTTTGACCCAAC
GTCTGGGTGAACTTTCTCAGCAGGTGGCCGAGTTGCGAGTACAAACTGAGTCTGCTGTCGGCACGGCAAA
GTCTAAATAAAAAAAATTCCAGAATCAATGAATAAATAAACGAGCTTGTTGTTGATTTAAAATCAAGTGT
TTTTATTTCATTTTTCGCGCACGGTATGCCCTGGACCACCGATCTCGATCATTGAGAACTCGGTGGATTT
TTTCCAGAATCCTATAGAGGTGGGATTGAATGTTTAGATACATGGGCATTAGGCCGTCTTTGGGGTGGAG
ATAGCTCCATTGAAGGGATTCATGCTCCGGGGTAGTGTTGTAAATCACCCAGTCATAACAAGGTCGCAGT
GCATGGTGTTGCACAATATCTTTTAGAAGTAGGCTGATTGCCACAGATAAGCCCTTGGTGTAGGTGTTTA
CAAACCGGTTGAGCTGGGAGGGGTGCATTCGAGGTGAAATTATGTGCATTTTGGATTGGATTTTTAAGTT
GGCAATATTGCCGCCAAGATCCCGTCTTGGGTTCATGTTATGAAGGACTACCAAGACGGTGTATCCGGTA
CATTTAGGAAATTTATCGTGCAGCTTGGATGGAAAAGCGTGGAAAAATTTGGAGACACCCTTGTGTCCTC
CGAGATTTTCCATGCACTCATCCATGATAATAGCAATGGGGCCGTGGGCAGCGGCGCGGGCAAACACGTT
CCGTGGGTCTGACACATCATAGTTATGTTCCTGAGTTAAATCATCATAAGCCATTTTAATGAATTTGGGG
CGGAGCGTACCAGATTGGGGTATGAATGTTCCTTCGGGCCCCGGAGCATAGTTCCCCTCACAGATTTGCA
TTTCCCAAGCTTTCAGTTCTGAGGGTGGAATCATGTCCACCTGGGGGGCTATGAAGAACACCGTTTCGGG
GGCGGGGGTGATTAGTTGGGATGATAGCAAGTTTCTGAGCAATTGAGATTTGCCACATCCGGTGGGGCCA
TAAATAATTCCGATTACAGGTTGCAGGTGGTAGTTTAGGGAACGGCAACTGCCGTCTTCTCGAAGCAAGG
GGGCCACCTCGTTCATCATTTCCCTTACATGCATATTTTCCCGCACCAAATCCATTAGGAGGCGCTCTCC
TCCTAGTGATAGAAGTTCTTGTAGTGAGGAAAAGTTTTTCAGCGGTTTTAGACCGTCAGCCATGGGCATT
TTGGAAAGAGTTTGCTGCAAAAGTTCTAGTCTGTTCCACAGTTCAGTGATGTGTTCTATGGCATCTCGAT
CCAGCAGACCTCCTCGTTTCGCGGGTTTGGACGGCTCCTGGAGTAGGGTATGAGACGATGGGCGTCCAGC
GCTGCCAGGGTTCGGTCCTTCCAGGGTCTCAGTGTTCGAGTCAGGGTTGTTTCCGTCACAGTGAAGGGGT
GTGCGCCTGCTTGGGCGCTTGCCAGGGTGCGCTTCAGACTCATTCTGCTGGTGGAGAACTTCTGTCGCTT
GGCGCCCTGTATGTCGGCCAAGTAGCAGTTTACCATGAGTTCGTAGTTGAGCGCCTCGGCTGCGTGGCCT
TTGGCGCGGAGCTTACCTTTGGAAGTTTTCTTGCATACCGGGCAGTATAGGCATTTCAGCGCATACAGCT
TGGGCGCAAGGAAAATGGATTCTGGGGAGTATGCATCCGCGCCGCAGGAGGCGCAAACAGTTTCACATTC
CACCAGCCAGGTTAAATCCGGTTCATTGGGGTCAAAAACAAGTTTTCCGCCATATTTTTTGATGCGTTTC
TTACCTTTGGTCTCCATAAGTTCGTGTCCTCGTTGAGTGACAAACAGGCTGTCCGTATCTCCGTAGACTG
ATTTTACAGGCCTCTTCTCCAGTGGAGTGCCTCGGTCTTCTTCGTACAGGAACTCTGACCACTCTGATAC
AAAGGCGCGCGTCCAGGCCAGCACAAAGGAGGCTATGTGGGAGGGGTAGCGATCGTTGTCAACCAGGGGG
TCCACCTTTTCCAAAGTATGCAAACACATGTCACCCTCTTCAACATCCAGGAATGTGATTGGCTTGTAGG
TGTATTTCACGTGACCTGGGGTCCCCGCTGGGGGGGTATAAAAGGGGGCGGTTCTTTGCTCTTCCTCACT
GTCTTCCGGATCGCTGTCCAGGAACGTCAGCTGTTGGGGTAGGTATTCCCTCTCGAAGGCGGGCATGACC
TCTGCACTCAGGTTGTCAGTTTCTAAGAACGAGGAGGATTTGATATTGACAGTGCCGGTTGAGATGCCTT
TCATGAGGTTTTCGTCCATTTGGTCAGAAAACACAATTTTTTTATTGTCAAGTTTGGTGGCAAATGATCC
ATACAGGGCGTTGGATAAAAGTTTGGCAATGGATCGCATGGTTTGGTTCTTTTCCTTGTCCGCGCGCTCT
TTGGCGGCGATGTTGAGTTGGACATACTCGCGTGCCAGGCACTTCCATTCGGGGAAGATAGTTGTTAATT
CATCTGGCACGATTCTCACTTGCCACCCTCGATTATGCAAGGTAATTAAATCCACACTGGTGGCCACCTC
GCCTCGAAGGGGTTCATTGGTCCAACAGAGCCTACCTCCTTTCCTAGAACAGAAAGGGGGAAGTGGGTCT
AGCATAAGTTCATCGGGAGGGTCTGCATCCATGGTAAAGATTCCCGGAAGTAAATCCTTATCAAAATAGC
TGATGGGAGTGGGGTCATCTAAGGCCATTTGCCATTCTCGAGCTGCCAGTGCGCGCTCATATGGGTTAAG
GGGACTGCCCCAGGGCATGGGATGGGTGAGAGCAGAGGCATACATGCCACAGATGTCATAGACGTAGATG
GGATCCTCAAAGATGCCTATGTAGGTTGGATAGCATCGCCCCCCTCTGATACTTGCTCGCACATAGTCAT
ATAGTTCATGTGATGGCGCTAGCAGCCCCGGACCCAAGTTGGTGCGATTGGGTTTTTCTGTTCTGTAGAC
GATCTGGCGAAAGATGGCGTGAGAATTGGAAGAGATGGTGGGTCTTTGAAAAATGTTGAAATGGGCATGA
GGTAGACCTACAGAGTCTCTGACAAAGTGGGCATAAGATTCTTGAAGCTTGGTTACCAGTTCGGCGGTGA
CAAGTACGTCTAGGGCGCAGTAGTCAAGTGTTTCTTGAATGATGTCATAACCTGGTTGGTTTTTCTTTTC
CCACAGTTCGCGGTTGAGAAGGTATTCTTCGCGATCCTTCCAGTACTCTTCTAGCGGAAACCCGTCTTTG
TCTGCACGGTAAGATCCTAGCATGTAGAACTGATTAACTGCCTTGTAAGGGCAGCAGCCCTTCTCTACGG
GTAGAGAGTATGCTTGAGCAGCTTTTCGTAGCGAAGCGTGAGTAAGGGCAAAGGTGTCTCTGACCATGAC
TTTGAGAAATTGGTATTTGAAGTCCATGTCGTCACAGGCTCCCTGTTCCCAGAGTTGGAAGTCTACCCGT
TTCTTGTAGGCGGGGTTGGGCAAAGCGAAAGTAACATCATTGAAGAGAATCTTACCGGCTCTGGGCATAA
AATTGCGAGTGATGCGGAAAGGCTGTGGTACTTCCGCTCGATTGTTGATCACCTGGGCAGCTAGGACGAT
TTCGTCGAAACCGTTGATGTTGTGTCCTACGATGTATAATTCTATGAAACGCGGCGTGCCTCTGACGTGA
GGTAGCTTACTGAGCTCATCAAAGGTTAGGTCTGTGGGGTCAGATAAGGCGTAGTGTTCGAGAGCCCATT
CGTGCAGGTGAGGATTTGCATGTAGGAATGATGACCAAAGATCTACCGCCAGTGCTGTTTGTAACTGGTC
CCGATACTGACGAAAATGCCGGCCAATTGCCATTTTTTCTGGAGTGACACAGTAGAAGGTTCTGGGGTCT
TGTTGCCATCGATCCCACTTGAGTTTAATGGCTAGATCGTGGGCCATGTTGACGAGACGCTCTTCTCCTG
AGAGTTTCATGACCAGCATGAAAGGAACTAGTTGTTTGCCAAAGGATCCCATCCAGGTGTAAGTTTCCAC
ATCGTAGGTCAGGAAGAGTCTTTCTGTGCGAGGATGAGAGCCGATCGGGAAGAACTGGATTTCCTGCCAC
CAGTTGGAGGATTGGCTGTTGATGTGATGGAAGTAGAAGTTTCTGCGGCGCGCCGAGCATTCGTGTTTGT
GCTTGTACAGACGGCCGCAGTAGTCGCAGCGTTGCACGGGTTGTATCTCGTGAATGAGCTGTACCTGGCT
TCCCTTGACGAGAAATTTCAGTGGGAAGCCGAGGCCTGGCGATTGTATCTCGTGCTCTTCTATATTCGCT
GTATCGGCCTGTTCATCTTCTGTTTCGATGGTGGTCATGCTGACGAGCCCCCGCGGGAGGCAAGTCCAGA
CCTCGGCGCGGGAGGGGCGGAGCTGAAGGACGAGAGCGCGCAGGCTGGAGCTGTCCAGAGTCCTGAGACG
CTGCGGACTCAGGTTAGTAGGTAGGGACAGAAGATTAACTTGCATGATCTTTTCCAGGGCGTGCGGGAGG
TTCAGATGGTACTTGATTTCCACAGGTTCGTTTGTAGAGACGTCAATGGCTTGCAGGGTTCCGTGTCCTT
TGGGCGCCACTACCGTACCTTTGTTTTTTCTTTTGATCGGTGGTGGCTCTCTTGCTTCTTGCATGCTCAG
AAGCGGTGACGGGGACGCGCGCCGGGCGGCAGCGGTTGTTCCGGACCCGGGGGCATGGCTGGTAGTGGCA
CGTCGGCGCCGCGCACGGGCAGGTTCTGGTATTGCGCTCTGAGAAGACTTGCGTGCGCCACCACGCGTCG
ATTGACGTCTTGTATCTGACGTCTCTGGGTGAAAGCTACCGGCCCCGTGAGCTTGAACCTGAAAGAGAGT
TCAACAGAATCAATTTCGGTATCGTTAACGGCAGCTTGTCTCAGTATTTCTTGTACGTCACCAGAGTTGT
CCTGGTAGGCGATCTCCGCCATGAACTGCTCGATTTCTTCCTCCTGAAGATCTCCGCGACCCGCTCTTTC
GACGGTGGCCGCGAGGTCATTGGAGATACGGCCCATGAGTTGGGAGAATGCATTCATGCCCGCCTCGTTC
CAGACGCGGCTGTAAACCACGGCCCCCTCGGAGTCTCTTGCGCGCATCACCACCTGAGCGAGGTTAAGCT
CCACGTGTCTGGTGAAGACCGCATAGTTGCATAGGCGCTGAAAAAGGTAGTTGAGTGTGGTGGCAATGTG
TTCGGCGACGAAGAAATACATGATCCATCGTCTCAGCGGCATTTCGCTAACATCGCCCAGAGCTTCCAAG
CGCTCCATGGCCTCGTAGAAGTCCACGGCAAAATTAAAAAACTGGGAGTTTCGCGCGGACACGGTCAATT
CCTCCTCGAGAAGACGGATGAGTTCGGCTATGGTGGCCCGTACTTCGCGTTCGAAGGCTCCCGGGATCTC
TTCTTCCTCTTCTATCTCTTCTTCCACTAACATCTCTTCTTCGTCTTCAGGCGGGGGCGGAGGGGGCACG
CGGCGACGTCGACGGCGCACGGGCAAACGGTCGATGAATCGTTCAATGACCTCTCCGCGGCGGCGGCGCA
TGGTTTCAGTGACGGCGCGGCCGTTCTCGCGCGGTCGCAGAGTAAAAACACCGCCGCGCATCTCCTTAAA
GTGGTGACTGGGAGGTTCTCCGTTTGGGAGGGAGAGGGCGCTGATTATACATTTTATTAATTGGCCCGTA
GGGACTGCGCGCAGAGATCTGATCGTGTCAAGATCCACGGGATCTGAAAACCTTTCGACGAAAGCGTCTA
ACCAGTCACAGTCACAAGGTAGGCTGAGTACGGCTTCTTGTGGGCGGGGGTGGTTATGTGTTCGGTCTGG
GTCTTCTGTTTCTTCTTCATCTCGGGAAGGTGAGACGATGCTGCTGGTGATGAAATTAAAGTAGGCAGTT
CTAAGACGGCGGATGGTGGCGAGGAGCACCAGGTCTTTGGGTCCGGCTTGCTGGATACGCAGGCGATTGG
CCATTCCCCAAGCATTATCCTGACATCTAGCAAGATCTTTGTAGTAGTCTTGCATGAGCCGTTCTACGGG
CACTTCTTCCTCACCCGTTCTGCCATGCATACGTGTGAGTCCAAATCCGCGCATTGGTTGTACCAGTGCC
AAGTCAGCTACGACTCTTTCGGCGAGGATGGCTTGCTGTACTTGGGTAAGGGTGGCTTGAAAGTCATCAA
AATCCACAAAGCGGTGGTAAGCCCCTGTATTAATGGTGTAAGCACAGTTGGCCATGACTGACCAGTTAAC
TGTCTGGTGACCAGGGCGCACGAGCTCGGTGTATTTAAGGCGCGAATAGGCGCGGGTGTCAAAGATGTAA
TCGTTGCAGGTGCGCACCAGATACTGGTACCCTATAAGAAAATGCGGCGGTGGTTGGCGGTAGAGAGGCC
ATCGTTCTGTAGCTGGAGCGCCAGGGGCGAGGTCTTCCAACATAAGGCGGTGATAGCCGTAGATGTACCT
GGACATCCAGGTGATTCCTGCGGCGGTAGTAGAAGCCCGAGGAAACTCGCGTACGCGGTTCCAAATGTTG
CGTAGCGGCATGAAGTAGTTCATTGTAGGCACGGTTTGACCAGTGAGGCGCGCGCAGTCATTGATGCTCT
ATAGACACGGAGAAAATGAAAGCGTTCAGCGACTCGACTCCGTAGCCTGGAGGAACGTGAACGGGTTGGG
TCGCGGTGTACCCCGGTTCGAGACTTGTACTCGAGCCGGCCGGAGCCGCGGCTAACGTGGTATTGGCACT
CCCGTCTCGACCCAGCCTACAAAAATCCAGGATACGGAATCGAGTCGTTTTGCTGGTTTCCGAATGGCAG
GGAAGTGAGTCCTATTTTTTTTTTTTTTGCCGCTCAGATGCATCCCGTGCTGCGACAGATGCGCCCCCAA
CAACAGCCCCCCTCGCAGCAGCAGCAGCAGCAACCACAAAAGGCTGTCCCTGCAACTACTGCAACTGCCG
CCGTGAGCGGTGCGGGACAGCCCGCCTATGATCTGGACTTGGAAGAGGGCGAAGGACTGGCACGTCTAGG
TGCGCCTTCGCCCGAGCGGCATCCGCGAGTTCAACTGAAAAAAGATTCTCGCGAGGCGTATGTGCCCCAA
CAGAACCTATTTAGAGACAGAAGCGGCGAGGAGCCGGAGGAGATGCGAGCTTCCCGCTTTAACGCGGGTC
GTGAGCTGCGTCACGGTTTGGACCGAAGACGAGTGTTGCGAGACGAGGATTTCGAAGTTGATGAAGTGAC
AGGGATCAGTCCTGCCAGGGCACACGTGGCTGCAGCCAACCTTGTATCGGCTTACGAGCAGACAGTAAAG
GAAGAGCGTAACTTCCAAAAGTCTTTTAATAATCATGTGCGAACCCTGATTGCCCGCGAAGAAGTTACCC
TTGGTTTGATGCATTTGTGGGATTTGATGGAAGCTATCATTCAGAACCCTACTAGCAAACCTCTGACCGC
CCAGCTGTTTCTGGTGGTGCAACACAGCAGAGACAATGAGGCTTTCAGAGAGGCGCTGCTGAACATCACC
GAACCCGAGGGGAGATGGTTGTATGATCTTATCAACATTCTACAGAGTATCATAGTGCAGGAGCGGAGCC
TGGGCCTGGCCGAGAAGGTAGCTGCCATCAATTACTCGGTTTTGAGCTTGGGAAAATATTACGCTCGCAA
AATCTACAAGACTCCATACGTTCCCATAGACAAGGAGGTGAAGATAGATGGGTTCTACATGCGCATGACG
CTCAAGGTCTTGACCCTGAGCGATGATCTTGGGGTGTATCGCAATGACAGAATGCATCGCGCGGTTAGCG
CCAGCAGGAGGCGCGAGTTAAGCGACAGGGAACTGATGCACAGTTTGCAAAGAGCTCTGACTGGAGCTGG
AACCGAGGGTGAGAATTACTTCGACATGGGAGCTGACTTGCAGTGGCAGCCTAGTCGCAGGGCTCTGAGC
GCCGCGACGGCAGGATGTGAGCTTCCTTACATAGAAGAGGCGGATGAAGGCGAGGAGGAAGAGGGCGAGT
ACTTGGAAGACTGATGGCACAACCCGTGTTTTTTGCTAGATGGAACAGCAAGCACCGGATCCCGCAATGC
GGGCGGCGCTGCAGAGCCAGCCGTCCGGCATTAACTCCTCGGACGATTGGACCCAGGCCATGCAACGTAT
CATGGCGTTGACGACTCGCAACCCCGAAGCCTTTAGACAGCAACCCCAGGCCAACCGTCTATCGGCCATC
ATGGAAGCTGTAGTGCCTTCCCGATCTAATCCCACTCATGAGAAGGTCCTGGCCATCGTGAACGCGTTGG
TGGAGAACAAAGCTATTCGTCCAGATGAGGCCGGACTGGTATACAACGCTCTCTTAGAACGCGTGGCTCG
CTACAACAGTAGCAATGTGCAAACCAATTTGGACCGTATGATAACAGATGTACGCGAAGCCGTGTCTCAG
CGCGAAAGGTTCCAGCGTGATGCCAACCTGGGTTCGCTGGTGGCGTTAAATGCTTTCTTGAGTACTCAGC
CTGCTAATGTGCCGCGTGGTCAACAGGATTATACTAACTTTTTAAGTGCTTTGAGACTGATGGTATCAGA
AGTACCTCAGAGCGAAGTGTATCAGTCCGGTCCTGATTACTTCTTTCAGACTAGCAGACAGGGCTTGCAG
ACGGTAAATCTGAGCCAAGCTTTTAAAAACCTTAAAGGTTTGTGGGGAGTGCATGCCCCGGTAGGAGAAA
GAGCAACCGTGTCTAGCTTGTTAACTCCGAACTCCCGCCTGTTATTACTGTTGGTAGCTCCTTTCACCGA
CAGCGGTAGCATCGACCGTAATTCCTATTTGGGTTACCTACTAAACCTGTATCGCGAAGCCATAGGGCAA
AGTCAGGTGGACGAGCAGACCTATCAAGAAATTACCCAAGTCAGTCGCGCTTTGGGACAGGAAGACACTG
GCAGTTTGGAAGCCACTCTGAACTTCTTGCTTACCAATCGGTCTCAAAAGATCCCTCCTCAATATGCTCT
TACTGCGGAGGAGGAGAGGATCCTTAGATATGTGCAGCAGAGCGTGGGATTGTTTCTGATGCAAGAGGGG
GCAACTCCGACTGCAGCACTGGACATGACAGCGCGAAATATGGAGCCCAGCATGTATGCCAGTAACCGAC
CTTTCATTAACAAACTGCTGGACTACTTGCACAGAGCTGCCGCTATGAACTCTGATTATTTCACCAATGC
CATCTTAAACCCGCACTGGCTGCCCCCACCTGGTTTCTACACGGGCGAATATGACATGCCCGACCCTAAT
GACGGATTTCTGTGGGACGACGTGGACAGCGATGTTTTTTCACCTCTTTCTGATCATCGCACGTGGAAAA
AGGAAGGCGGTGATAGAATGCATTCTTCTGCATCGCTGTCCGGGGTCATGGGTGCTACCGCGGCTGAGCC
CGAGTCTGCAAGTCCTTTTCCTAGTCTACCCTTTTCTCTACACAGTGTACGTAGCAGCGAAGTGGGTAGA
ATAAGTCGCCCGAGTTTAATGGGCGAAGAGGAGTACCTAAACGATTCCTTGCTCAGACCGGCAAGAGAAA
AAAATTTCCCAAACAATGGAATAGAAAGTTTGGTGGATAAAATGAGTAGATGGAAGACTTATGCTCAGGA
TCACAGAGACGAGCCTGGGATCATGGGGACTACAAGTAGAGCGAGCCGTAGACGCCAGCGCCATGACAGA
CAGAGGGGTCTTGTGTGGGACGATGAGGATTCGGCCGATGATAGCAGCGTGTTGGACTTGGGTGGGAGAG
GAAGGGGCAACCCGTTTGCTCATTTGCGCCCTCGCTTGGGTGGTATGTTGTGAAAAAAAATAAAAAAGAA
AAACTCACCAAGGCCATGGCGACGAGCGTACGTTCGTTCTTCTTTATTATCTGTGTCTAGTATAATGAGG
CGAGTCGTGCTAGGCGGAGCGGTGGTGTATCCGGAGGGTCCTCCTCCTTCGTACGAGAGCGTGATGCAGC
AGCAGCAGGCGACGGCGGTGATGCAATCCCCACTGGAGGCTCCCTTTGTGCCTCCGCGATACCTGGCACC
TACGGAGGGCAGAAACAGCATTCGTTACTCGGAACTGGCACCTCAGTACGATACCACCAGGTTGTATCTG
GTGGACAACAAGTCGGCGGACATTGCTTCTCTGAACTATCAGAATGACCACAGCAACTTCTTGACCACGG
TGGTGCAGAACAATGACTTTACCCCTACGGAAGCCAGCACCCAGACCATTAACTTTGATGAACGATCGCG
GTGGGGCGGTCAGCTAAAGACCATCATGCATACTAACATGCCAAACGTGAACGAGTATATGTTTAGTAAC
AAGTTCAAAGCGCGTGTGATGGTGTCCAGAAAACCTCCCGACGGTGCTGCAGTTGGGGATACTTATGATC
ACAAGCAGGATATTTTGGAATATGAGTGGTTCGAGTTTACTTTGCCAGAAGGCAACTTTTCAGTTACTAT
GACTATTGATTTGATGAACAATGCCATCATAGATAATTACTTGAAAGTGGGTAGACAGAATGGAGTGCTT
GAAAGTGACATTGGTGTTAAGTTCGACACCAGGAACTTCAAGCTGGGATGGGATCCCGAAACCAAGTTGA
TCATGCCTGGAGTGTATACGTATGAAGCCTTCCATCCTGACATTGTCTTACTGCCTGGCTGCGGAGTGGA
TTTTACCGAGAGTCGTTTGAGCAACCTTCTTGGTATCAGAAAAAAACAGCCATTTCAAGAGGGTTTTAAG
ATTTTGTATGAAGATTTAGAAGGTGGTAATATTCCGGCCCTCTTGGATGTAGATGCCTATGAGAACAGTA
AGAAAGAACAAAAAGCCAAAATAGAAGCTGCTACAGCTGCTGCAGAAGCTAAGGCAAACATAGTTGCCAG
CGACTCTACAAGGGTTGCTAACGCTGGAGAGGTCAGAGGAGACAATTTTGCGCCAACACCTGTTCCGACT
GCAGAATCATTATTGGCCGATGTGTCTGAAGGAACGGACGTGAAACTCACTATTCAACCTGTAGAAAAAG
ATAGTAAGAATAGAAGCTATAATGTGTTGGAAGACAAAATCAACACAGCCTATCGCAGTTGGTATCTTTC
GTACAATTATGGCGATCCCGAAAAAGGAGTGCGTTCCTGGACATTGCTCACCACCTCAGATGTCACCTGC
GGAGCAGAGCAGGTTTACTGGTCGCTTCCAGACATGATGAAGGATCCTGTCACTTTCCGCTCCACTAGAC
AAGTCAGTAACTACCCTGTGGTGGGTGCAGAGCTTATGCCCGTCTTCTCAAAGAGCTTCTACAACGAACA
AGCTGTGTACTCCCAGCAGCTCCGCCAGTCCACCTCGCTTACGCACGTCTTCAACCGCTTTCCTGAGAAC
CAGATTTTAATCCGTCCGCCGGCGCCCACCATTACCACCGTCAGTGAAAACGTTCCTGCTCTCACAGATC
ACGGGACCCTGCCGTTGCGCAGCAGTATCCGGGGAGTCCAACGTGTGACCGTTACTGACGCCAGACGCCG
CACCTGTCCCTACGTGTACAAGGCACTGGGCATAGTCGCACCGCGCGTCCTTTCAAGCCGCACTTTCTAA
AAAAAAAATGTCCATTCTTATCTCGCCCAGTAATAACACCGGTTGGGGTCTGCGCGCTCCAAGCAAGATG
TACGGAGGCGCACGCAAACGTTCTACCCAACATCCCGTGCGTGTTCGCGGACATTTTCGCGCTCCATGGG
GTGCCCTCAAGGGCCGCACTCGCGTTCGAACCACCGTCGATGATGTAATCGATCAGGTGGTTGCCGACGC
CCGTAATTATACTCCTACTGCGCCTACATCTACTGTGGATGCAGTTATTGACAGTGTAGTGGCTGACGCT
CGCAACTATGCTCGACGTAAGAGCCGGCGAAGGCGCATTGCCAGACGCCACCGAGCTACCACTGCCATGC
GAGCCGCAAGAGCTCTGCTACGAAGAGCTAGACGCGTGGGGCGAAGAGCCATGCTTAGGGCGGCCAGACG
TGCAGCTTCGGGCGCCAGCGCCGGCAGGTCCCGCAGGCAAGCAGCCGCTGTCGCAGCGGCGACTATTGCC
GACATGGCCCAATCGCGAAGAGGCAATGTATACTGGGTGCGTGACGCTGCCACCGGTCAACGTGTACCCG
TGCGCACCCGTCCCCCTCGCACTTAGAAGATACTGAGCAGTCTCCGATGTTGTGTCCCAGCGGCGAGGAT
GTCCAAGCGCAAATACAAGGAAGAAATGCTGCAGGTTATCGCACCTGAAGTCTACGGCCAACCGTTGAAG
GATGAAAAAAAACCCCGCAAAATCAAGCGGGTTAAAAAGGACAAAAAAGAAGAGGAAGATGGCGATGATG
GGCTGGCGGAGTTTGTGCGCGAGTTTGCCCCACGGCGACGCGTGCAATGGCGTGGGCGCAAAGTTCGACA
TGTGTTGAGACCTGGAACTTCGGTGGTCTTTACACCCGGCGAGCGTTCAAGCGCTACTTTTAAGCGTTCC
TATGATGAGGTGTACGGGGATGATGATATTCTTGAGCAGGCGGCTGACCGATTAGGCGAGTTTGCTTATG
GCAAGCGTAGTAGAATAACTTCCAAGGATGAGACAGTGTCAATACCCTTGGATCATGGAAATCCCACCCC
TAGTCTTAAACCGGTCACTTTGCAGCAAGTGTTACCCGTAACTCCGCGAACAGGTGTTAAACGCGAAGGT
GAAGATTTGTATCCCACTATGCAACTGATGGTACCCAAACGCCAGAAGTTGGAGGACGTTTTGGAGAAAG
TAAAAGTGGATCCAGATATTCAACCTGAGGTTAAAGTGAGACCCATTAAGCAGGTAGCGCCTGGTCTGGG
GGTACAAACTGTAGACATTAAGATTCCCACTGAAAGTATGGAAGTGCAAACTGAACCCGCAAAGCCTACT
GCCACCTCCACTGAAGTGCAAACGGATCCATGGATGCCCATGCCTATTACAACTGACGCCGCCGGTCCCA
CTCGAAGATCCCGACGAAAGTACGGTCCAGCAAGTCTGTTGATGCCCAATTATGTTGTACACCCATCTAT
TATTCCTACTCCTGGTTACCGAGGCACTCGCTACTATCGCAGCCGAAACAGTACCTCCCGCCGTCGCCGC
AAGACACCTGCAAATCGCAGTCGTCGCCGTAGACGCACAAGCAAACCGACTCCCGGCGCCCTGGTGCGGC
AAGTGTACCGCAATGGTAGTGCGGAACCTTTGACACTGCCGCGTGCGCGTTACCATCCGAGTATCATCAC
TTAATCAATGTTGCCGCTGCCTCCTTGCAGATATGGCCCTCACTTGTCGCCTTCGCGTTCCCATCACTGG
TTACCGAGGAAGAAACTCGCGCCGTAGAAGAGGGATGTTGGGACGCGGAATGCGACGCTACAGGCGACGG
CGTGCTATCCGCAAGCAATTGCGGGGTGGTTTTTTACCAGCCTTAATTCCAATTATCGCTGCTGCAATTG
GCGCGATACCAGGCATAGCTTCCGTGGCGGTTCAGGCCTCGCAACGACATTGACATTGGAAAAAAAACGT
ATAAATAAAAAAAAATACAATGGACTCTGACACTCCTGGTCCTGTGACTATGTTTTCTTAGAGATGGAAG
ACATCAATTTTTCATCCTTGGCTCCGCGACACGGCACGAAGCCGTACATGGGCACCTGGAGCGACATCGG
CACGAGCCAACTGAACGGGGGCGCCTTCAATTGGAGCAGTATCTGGAGCGGGCTTAAAAATTTTGGCTCA
ACCATAAAAACATACGGGAACAAAGCTTGGAACAGCAGTACAGGACAGGCGCTTAGAAATAAACTTAAAG
ACCAGAACTTCCAACAAAAAGTAGTCGATGGGATAGCTTCCGGCATCAATGGAGTGGTAGATTTGGCTAA
CCAGGCTGTGCAGAAAAAGATAAACAGTCGTTTGGACCCGCCGCCAGCAACCCCAGGTGAAATGCAAGTG
GAGGAAGAAATTCCTCCGCCAGAAAAACGAGGCGACAAGCGTCCGCGTCCCGATTTGGAAGAGACGCTGG
TGACGCGCGTAGATGAACCGCCTTCTTATGAGGAAGCAACGAAGCTTGGAATGCCCACCACTAGACCGAT
AGCCCCAATGGCCACCGGGGTGATGAAACCTTCTCAGTTGCATCGACCCGTCACCTTGGATTTGCCCCCT
CCCCCTGCTGCTACTGCTGTACCCGCTTCTAAGCCTGTCGCTGCCCCGAAACCAGTCGCCGTAGCCAGGT
CACGTCCCGGGGGCGCTCCTCGTCCAAATGCGCACTGGCAAAATACTCTGAACAGCATCGTGGGTCTAGG
CGTGCAAAGTGTAAAACGCCGTCGCTGCTTTTAATTAAATATGGAGTAGCGCTTAACTTGCCTATCTGTG
TATATGTGTCATTACACGCCGTCACAGCAGCAGAGGAAAAAAGGAAGAGGTCGTGCGTCGACGCTGAGTT
ACTTTCAAGATGGCCACCCCATCGATGCTGCCCCAATGGGCATACATGCACATCGCCGGACAGGATGCTT
CGGAGTACCTGAGTCCGGGTCTGGTGCAGTTCGCCCGCGCCACAGACACCTACTTCAATCTGGGAAATAA
GTTTAGAAATCCCACCGTAGCGCCGACCCACGATGTGACCACCGACCGTAGCCAGCGGCTCATGTTGCGC
TTCGTGCCCGTTGACCGGGAGGACAATACATACTCTTACAAAGTGCGGTACACCCTGGCCGTGGGCGACA
ACAGAGTGCTGGATATGGCCAGCACGTTCTTTGACATTAGGGGCGTGTTGGACAGAGGTCCCAGTTTCAA
ACCCTATTCTGGTACGGCTTACAACTCTCTGGCTCCTAAAGGCGCTCCAAATGCATCTCAATGGATTGCA
AAAGGCGTACCAACTGCAGCAGCCGCAGGCAATGGTGAAGAAGAACATGAAACAGAGGAGAAAACTGCTA
CTTACACTTTTGCCAATGCTCCTGTAAAAGCCGAGGCTCAAATTACAAAAGAGGGCTTACCAATAGGTTT
GGAGATTTCAGCTGAAAACGAATCTAAACCCATCTATGCAGATAAACTTTATCAGCCAGAACCTCAAGTG
GGAGATGAAACTTGGACTGACCTAGACGGAAAAACCGAAGAGTATGGAGGCAGGGCTCTAAAGCCTACTA
CTAACATGAAACCCTGTTACGGGTCCTATGCGAAGCCTACTAATTTAAAAGGTGGTCAGGCAAAACCGAA
AAACTCGGAACCGTCGAGTGAAAAAATTGAATATGATATTGACATGGAATTTTTTGATAACTCATCGCAA
AGAACAAACTTCAGTCCTAAAATTGTCATGTATGCAGAAAATGTAGGTTTGGAAACGCCAGACACTCATG
TAGTGTACAAACCTGGAACAGAAGACACAAGTTCCGAAGCTAATTTGGGACAACAGTCTATGCCCAACAG
ACCCAACTACATTGGCTTCAGAGATAACTTTATTGGACTCATGTACTATAACAGTACTGGTAACATGGGG
GTGCTGGCTGGTCAAGCGTCTCAGTTAAATGCAGTGGTTGACTTGCAGGACAGAAACACAGAACTTTCTT
ACCAACTCTTGCTTGACTCTCTGGGCGACAGAACCAGATACTTTAGCATGTGGAATCAGGCTGTGGACAG
TTATGATCCTGATGTACGTGTTATTGAAAATCATGGTGTGGAAGATGAACTTCCCAACTATTGTTTTCCA
CTGGACGGCATAGGTGTTCCAACAACCAGTTACAAATCAATAGTTCCAAATGGAGAAGATAATAATAATT
GGAAAGAACCTGAAGTAAATGGAACAAGTGAGATCGGACAGGGTAATTTGTTTGCCATGGAAATTAACCT
TCAAGCCAATCTATGGCGAAGTTTCCTTTATTCCAATGTGGCTCTGTATCTCCCAGACTCGTACAAATAC
ACCCCGTCCAATGTCACTCTTCCAGAAAACAAAAACACCTACGACTACATGAACGGGCGGGTGGTGCCGC
CATCTCTAGTAGACACCTATGTGAACATTGGTGCCAGGTGGTCTCTGGATGCCATGGACAATGTCAACCC
ATTCAACCACCACCGTAACGCTGGCTTGCGTTACCGATCTATGCTTCTGGGTAACGGACGTTATGTGCCT
TTCCACATACAAGTGCCTCAAAAATTCTTCGCTGTTAAAAACCTGCTGCTTCTCCCAGGCTCCTACACTT
ATGAGTGGAACTTTAGGAAGGATGTGAACATGGTTCTACAGAGTTCCCTCGGTAACGACCTGCGGGTAGA
TGGCGCCAGCATCAGTTTCACGAGCATCAACCTCTATGCTACTTTTTTCCCCATGGCTCACAACACCGCT
TCCACCCTTGAAGCCATGCTGCGGAATGACACCAATGATCAGTCATTCAACGACTACCTATCTGCAGCTA
ACATGCTCTACCCCATTCCTGCCAATGCAACCAATATTCCCATTTCCATTCCTTCTCGCAACTGGGCGGC
TTTCAGAGGCTGGTCATTTACCAGACTGAAAACCAAAGAAACTCCCTCTTTGGGGTCTGGATTTGACCCC
TACTTTGTCTATTCTGGTTCTATTCCCTACCTGGATGGTACCTTCTACCTGAACCACACTTTTAAGAAGG
TTTCCATCATGTTTGACTCTTCAGTGAGCTGGCCTGGAAATGACAGGTTACTATCTCCTAACGAATTTGA
AATAAAGCGCACTGTGGATGGCGAAGGCTACAACGTAGCCCAATGCAACATGACCAAAGACTGGTTCTTG
GTACAGATGCTCGCCAACTACAACATCGGCTATCAGGGCTTCTACATTCCAGAAGGATACAAAGATCGCA
TGTATTCATTTTTCAGAAACTTCCAGCCCATGAGCAGGCAGGTGGTTGATGAGGTCAATTACAAAGACTT
CAAGGCCGTCGCCATACCCTACCAACACAACAACTCTGGCTTTGTGGGTTACATGGCTCCGACCATGCGC
CAAGGTCAACCCTATCCCGCTAACTATCCCTATCCACTCATTGGAACAACTGCCGTAAATAGTGTTACGC
AGAAAAAGTTCTTGTGTGACAGAACCATGTGGCGCATACCGTTCTCGAGCAACTTCATGTCTATGGGGGC
CCTTACAGACTTGGGACAGAATATGCTCTATGCCAACTCAGCTCATGCTCTGGACATGACCTTTGAGGTG
GATCCCATGGATGAGCCCACCCTGCTTTATCTTCTCTTCGAAGTTTTCGACGTGGTCAGAGTGCATCAGC
CACACCGCGGCATCATCGAGGCAGTCTACCTGCGTACACCGTTCTCGGCCGGTAACGCTACCACGTAAGA
AGCTTCTTGCTTCTTGCAAATAGCAGCTGCAACCATGGCCTGCGGATCCCAAAACGGCTCCAGCGAGCAA
GAGCTCAGAGCCATTGTCCAAGACCTGGGTTGCGGACCCTATTTTTTGGGAACCTACGATAAGCGCTTCC
CGGGGTTCATGGCCCCCGATAAGCTCGCCTGTGCCATTGTAAATACGGCCGGACGTGAGACGGGGGGAGA
GCACTGGTTGGCTTTCGGTTGGAACCCACGTTCTAACACCTGCTACCTTTTTGATCCTTTTGGATTCTCG
GATGATCGTCTCAAACAGATTTACCAGTTTGAATATGAGGGTCTCCTGCGCCGCAGCGCTCTTGCTACCA
AGGACCGCTGTATTACGCTGGAAAAATCTACCCAGACCGTGCAGGGCCCCCGTTCTGCCGCCTGCGGACT
TTTCTGCTGCATGTTCCTTCACGCCTTTGTGCACTGGCCTGACCGTCCCATGGACGGAAACCCCACCATG
AAATTGCTAACTGGAGTGCCAAACAACATGCTTCATTCTCCTAAAGTCCAGCCCACCCTGTGTGACAATC
AAAAAGCACTCTACCATTTTCTTAATACCCATTCGCCTTATTTTCGCTCTCATCGTACACACATCGAAAG
GGCCACTGCGTTCGACCGTATGGATGTTCAATAATGACTCATGTAAACAACGTGTTCAATAAACATCACT
TTATTTTTTTACATGTATCAAGGCTCTGGATTACTTATTTATTTACAAGTCGAATGGGTTCTGACGAGAA
TCAGAATGACCCGCAGGCAGTGATACGTTGCGGAACTGATACTTGGGTTGCCACTTGAATTCGGGAATCA
CCAACTTGGGAACCGGTATATCGGGCAGGATGTCACTCCACAGCTTTCTGGTCAGCTGCAAAGCTCCAAG
CAGGTCAGGAGCCGAAATCTTGAAATCACAATTAGGACCAGTGCTCTGAGCGCGAGAGTTGCGGTACACC
GGATTGCAGCACTGAAACACCATCAGCGACGGATGTCTCACGCTTGCCAGCACGGTGGGATCTGCAATCA
TGCCCACATCCAGATCTTCAGCATTGGCAATGCTGAACGGGGTCATCTTGCAGGTCTGCCTACCCATGGC
GGGCACCCAATTAGGCTTGTGGTTGCAATCGCAGTGCAGGGGGATCAGTATCATCTTGGCCTGATCCTGT
CTGATTCCTGGATACACGGCTCTCATGAAAGCATCATATTGCTTGAAAGCCTGCTGGGCTTTACTACCCT
CGGTATAAAACATCCCGCAGGACCTGCTCGAAAACTGGTTAGCTGCACAGCCGGCATCATTCACACAGCA
GCGGGCGTCATTGTTGGCTATTTGCACCACACTTCTGCCCCAGCGGTTTTGGGTGATTTTGGTTCGCTCG
GGATTCTCCTTTAAGGCTCGTTGTCCGTTCTCGCTGGCCACATCCATCTCGATAATCTGCTCCTTCTGAA
TCATAATATTGCCATGCAGGCACTTCAGCTTGCCCTCATAATCATTGCAGCCATGAGGCCACAACGCACA
GCCTGTACATTCCCAATTATGGTGGGCGATCTGAGAAAAAGAATGTATCATTCCCTGCAGAAATCTTCCC
ATCATCGTGCTCAGTGTCTTGTGACTAGTGAAAGTTAACTGGATGCCTCGGTGCTCTTCGTTTACGTACT
GGTGACAGATGCGCTTGTATTGTTCGTGTTGCTCAGGCATTAGTTTAAAACAGGTTCTAAGTTCGTTATC
CAGCCTGTACTTCTCCATCAGCAGACACATCACTTCCATGCCTTTCTCCCAAGCAGACACCAGGGGCAAG
CTAATCGGATTCTTAACAGTGCAGGCAGCAGCTCCTTTAGCCAGAGGGTCATCTTTAGCGATCTTCTCAA
TGCTTCTTTTGCCATCCTTCTCAACGATGCGCACGGGCGGGTAGCTGAAACCCACTGCTACAAGTTGCGC
CTCTTCTCTTTCTTCTTCGCTGTCTTGACTGATGTCTTGCATGGGGATATGTTTGGTCTTCCTTGGCTTC
TTTTTGGGGGGTATCGGAGGAGGAGGACTGTCGCTCCGTTCCGGAGACAGGGAGGATTGTGACGTTTCGC
TCACCATTACCAACTGACTGTCGGTAGAAGAACCTGACCCCACACGGCGACAGGTGTTTTTCTTCGGGGG
CAGAGGTGGAGGCGATTGCGAAGGGCTGCGGTCCGACCTGGAAGGCGGATGACTGGCAGAACCCCTTCCG
CGTTCGGGGGTGTGCTCCCTGTGGCGGTCGCTTAACTGATTTCCTTCGCGGCTGGCCATTGTGTTCTCCT
AGGCAGAGAAACAACAGACATGGAAACTCAGCCATTGCTGTCAACATCGCCACGAGTGCCATCACATCTC
GTCCTCAGCGACGAGGAAAAGGAGCAGAGCTTAAGCATTCCACCGCCCAGTCCTGCCACCACCTCTACCC
TAGAAGATAAGGAGGTCGACGCATCTCATGACATGCAGAATAAAAAAGCGAAAGAGTCTGAGACAGACAT
CGAGCAAGACCCGGGCTATGTGACACCGGTGGAACACGAGGAAGAGTTGAAACGCTTTCTAGAGAGAGAG
GATGAAAACTGCCCAAAACAGCGAGCAGATAACTATCACCAAGATGCTGGAAATAGGGATCAGAACACCG
ACTACCTCATAGGGCTTGACGGGGAAGACGCGCTCCTTAAACATCTAGCAAGACAGTCGCTCATAGTCAA
GGATGCATTATTGGACAGAACTGAAGTGCCCATCAGTGTGGAAGAGCTCAGCTGCGCCTACGAGCTTAAC
CTTTTTTCACCTCGTACTCCCCCCAAACGTCAGCCAAACGGCACCTGCGAGCCAAATCCTCGCTTAAACT
TTTATCCAGCTTTTGCTGTGCCAGAAGTACTGGCTACCTATCACATCTTTTTTAAAAATCAAAAAATTCC
AGTCTCCTGCCGCGCTAATCGCACCCGCGCCGATGCCCTACTCAATCTGGGACCTGGTTCACGCTTACCT
GATATAGCTTCCTTGGAAGAGGTTCCAAAGATCTTCGAGGGTCTGGGCAATAATGAGACTCGGGCCGCAA
ATGCTCTGCAAAAGGGAGAAAATGGCATGGATGAGCATCACAGCGTTCTGGTGGAATTGGAAGGCGATAA
TGCCAGACTCGCAGTACTCAAGCGAAGCGTCGAGGTCACACACTTCGCATATCCCGCTGTCAACCTGCCC
CCTAAAGTCATGACGGCGGTCATGGACCAGTTACTCATTAAGCGCGCAAGTCCCCTTTCAGAAGACATGC
ATGACCCAGATGCCTGTGATGAGGGTAAACCAGTGGTCAGTGATGAGCAGCTAACCCGATGGCTGGGCAC
CGACTCTCCCCGGGATTTGGAAGAGCGTCGCAAGCTTATGATGGCCGTGGTGCTGGTTACCGTAGAACTA
GAGTGTCTCCGACGTTTCTTTACCGATTCAGAAACCTTGCGCAAACTCGAAGAGAATCTGCACTACACTT
TTAGACACGGCTTTGTGCGGCAGGCATGCAAGATATCTAACGTGGAACTCACCAACCTGGTTTCCTACAT
GGGTATTCTGCATGAGAATCGCCTAGGACAAAGCGTGCTGCACAGCACCCTTAAGGGGGAAGCCCGCCGT
GATTACATCCGCGATTGTGTCTATCTCTACCTGTGCCACACGTGGCAAACCGGCATGGGTGTATGGCAGC
AATGTTTAGAAGAACAGAACTTGAAAGAGCTTGACAAGCTCTTACAGAAATCTCTTAAGGTTCTGTGGAC
AGGGTTCGACGAGCGCACCGTCGCTTCCGACCTGGCAGACCTCATCTTCCCAGAGCGTCTCAGGGTTACT
TTGCGAAACGGATTGCCTGACTTTATGAGCCAGAGCATGCTTAACAATTTTCGCTCTTTCATCCTGGAAC
GCTCCGGTATCCTGCCCGCCACCTGCTGCGCACTGCCCTCCGACTTTGTGCCTCTCACCTACCGCGAGTG
CCCCCCGCCGCTATGGAGTCACTGCTACCTGTTCCGTCTGGCCAACTATCTCTCCTACCACTCGGATGTG
ATCGAGGATGTGAGCGGAGACGGCTTGCTGGAGTGCCACTGCCGCTGCAATCTGTGCACGCCCCACCGGT
CCCTAGCTTGCAACCCCCAGTTGATGAGCGAAACCCAGATAATAGGCACCTTTGAATTGCAAGGCCCCAG
CAGCCAAGGCGATGGGTCTTCTCCTGGGCAAAGTTTAAAACTGACCCCGGGACTGTGGACCTCCGCCTAC
TTGCGCAAGTTTGCTCCGGAAGATTACCACCCCTATGAAATCAAGTTCTATGAGGACCAATCACAGCCTC
CAAAGGCCGAACTTTCGGCTTGCGTCATCACCCAGGGGGCAATTCTGGCCCAATTGCAAGCCATCCAAAA
ATCCCGCCAAGAATTTCTACTGAAAAAGGGTAAGGGGGTCTACCTTGACCCCCAGACCGGCGAGGAACTC
AACACAAGGTTCCCTCAGGATGTCCCAACGACGAGAAAACAAGAAGTTGAAGGTGCAGCCGCCGCCCCCA
GAAGATATGGAGGAAGATTGGGACAGTCAGGCAGAGGAGGCGGAGGAGGACAGTCTGGAGGACAGTCTGG
AGGAAGACAGTTTGGAGGAGGAAAACGAGGAGGCAGAGGAGGTGGAAGAAGTAACCGCCGACAAACAGTT
ATCCTCGGCTGCGGAGACAAGCAACAGCGCTACCATCTCCGCTCCGAGTCGAGGAACCCGGCGGCGTCCC
AGCAGTAGATGGGACGAGACCGGACGCTTCCCGAACCCAACCAGCGCTTCCAAGACCGGTAAGAAGGATC
GGCAGGGATACAAGTCCTGGCGGGGGCATAAGAATGCCATCATCTCCTGCTTGCATGAGTGCGGGGGCAA
CATATCCTTCACGCGGCGCTACTTGCTATTCCACCATGGGGTGAACTTTCCGCGCAATGTTTTGCATTAC
TACCGTCACCTCCACAGCCCCTACTATAGCCAGCAAATCCCGACAGTCTCGACAGATAAAGACAGCGGCG
GCGACCTCCAACAGAAAACCAGCAGCGGCAGTTAGAAAATACACAACAAGTGCAGCAACAGGAGGATTAA
AGATTACAGCCAACGAGCCAGCGCAAACCCGAGAGTTAAGAAATCGGATCTTTCCAACCCTGTATGCCAT
CTTCCAGCAGAGTCGGGGTCAAGAGCAGGAACTGAAAATAAAAAACCGATCTCTGCGTTCGCTCACCAGA
AGTTGTTTGTATCACAAGAGCGAAGATCAACTTCAGCGCACTCTCGAGGACGCCGAGGCTCTCTTCAACA
AGTACTGCGCGCTGACTCTTAAAGAGTAGGCAGCGACCGCGCTTATTCAAAAAAGGCGGGAATTACATCA
TCCTCGACATGAGTAAAGAAATTCCCACGCCTTACATGTGGAGTTATCAACCCCAAATGGGATTGGCAGC
AGGCGCCTCCCAGGACTACTCCACCCGCATGAATTGGCTCAGCGCCGGGCCTTCTATGATTTCTCGAGTT
AATGATATACGCGCCTACCGAAACCAAATACTTTTGGAACAGTCAGCTCTTACCACCACGCCCCGCCAAC
ACCTTAATCCCAGAAATTGGCCCGCCGCCCTAGTGTACCAGGAAAGTCCCGCTCCCACCACTGTATTACT
TCCTCGAGACGCCCAGGCCGAAGTCCAAATGACTAATGCAGGTGCGCAGTTAGCTGGCGGCTCCACCCTA
TGTCGTCACAGGCCTCGGCATAATATAAAACGCCTGATGATCAGAGGCCGAGGTATCCAGCTCAACGACG
AGTCGGTGAGCTCTCCGCTTGGTCTACGACCAGACGGAATCTTTCAGATTGCCGGCTGCGGGAGATCTTC
CTTCACCCCTCGTCAGGCTGTTCTGACTTTGGAAAGTTCGTCTTCGCAACCCCGCTCGGGCGGAATCGGG
ACCGTTCAATTTGTAGAGGAGTTTACTCCCTCTGTCTACTTCAACCCCTTCTCCGGATCTCCTGGGCACT
ACCCGGACGAGTTCATACCGAACTTCGACGCGATTAGCGAGTCAGTGGACGGCTACGATTGATGTCTGGT
GACGCGGCTGAGCTATCTCGGCTGCGACATCTAGACCACTGCCGCCGCTTTCGCTGCTTTGCCCGGGAAC
TTATTGAGTTCATCTACTTCGAACTCCCCAAGGATCACCCTCAAGGTCCGGCCCACGGAGTGCGGATTAC
TATCGAAGGCAAAATAGACTCTCGCCTGCAACGAATTTTCTCCCAGCGGCCCGTGCTGATCGAGCGAGAC
CAGGGAAACACCACGGTTAGTAATCAATTACGGGGTCATTAGTTCATAGCCCATATATGGAGTTAACTTG
TTTATTGCAGCTTATAATGGTTACAAATAAAGCAATAGCATCACAAATTTCACAAATAAAGCATTTTTTT
CACTGCATTCTAGTTGTGGTTTGTCCAAACTCATCAATGTATCTTATCATGTCTGCTCGAAGCGGCCGGC
CGCCCCGACTCTAGAGTCGCGGCCTCATTAGGAAGTTCCTATACTTTCTAGAGAATAGGAACTTCTCAGA
AGAACTCGTCAAGAAGGCGATAGAAGGCGATGCGCTGCGAATCGGGAGCGGCGATACCGTAAAGCACGAG
GAAGCGGTCAGCCCATTCGCCGCCAAGCTCTTCAGCAATATCACGGGTAGCCAACGCTATGTCCTGATAG
CGGTCCGCCACACCCAGCCGGCCACAGTCGATGAATCCAGAAAAGCGGCCATTTTCCACCATGATATTCG
GCAAGCAGGCATCGCCATGGGTCACGACGAGATCCTCGCCGTCGGGCATGCGCGCCTTGAGCCTGGCGAA
CAGTTCGGCTGGCGCGAGCCCCTGATGCTCTTCGTCCAGATCATCCTGATCGACAAGACCGGCTTCCATC
CGAGTACGTGCTCGCTCGATGCGATGTTTCGCTTGGTGGTCGAATGGGCAGGTAGCCGGATCAAGCGTAT
GCAGCCGCCGCATTGCATCAGCCATGATGGATACTTTCTCGGCAGGAGCAAGGTGAGATGACAGGAGATC
CTGCCCCGGCACTTCGCCCAATAGCAGCCAGTCCCTTCCCGCTTCAGTGACAACGTCGAGCACAGCTGCG
CAAGGAACGCCCGTCGTGGCCAGCCACGATAGCCGCGCTGCCTCGTCCTGCAGTTCATTCAGGGCACCGG
ACAGGTCGGTCTTGACAAAAAGAACCGGGCGCCCCTGCGCTGACAGCCGGAACACGGCGGCATCAGAGCA
GCCGATTGCCTGTTGTGCCCAGTCATAGCCGAATAGCCTCTCCACCCAAGCGGCCGGAGAACCTGCGTGC
AATCCATCTTGTTCAATGGCCGATCCCATAACACCCCTTGTATTACTGTTTATGTAAGCAGACAGTTTTA
CTGTTCGTGATGATATATTTTTATCTTGTGCAATGTAACAGGTTGTGGCCATAGCGGGCCCGGGATTTTC
CTCCACGTCCCCGCATGTTAGAAGACTTCCCCTGCCCTCGGCTCTGGAAGTTCCTATACTTTCTAGAGAA
TAGGAACTTCCCGCCAGAATGCGTTCGCACAGCCGCCAGCCGGTCACTCCGTTGATGGTTACTCGGAACA
GCAGGGAGCCGTCGGGGTTGATCAGGCGCTCGTCGATAATTTTGTTGCCGTTCCACAGGGTCCCTGTTAC
AGTGATCTTTTTGCCGTCGAACACGGCGATGCCTTCATACGGCCGTCCGAAATAGTCGATCATGTTCGGC
GTAACCCCGTCGATTACCAGTGTGCCATAGTGCAGGATCACCTTAAAGTGATGATCATCCACAGGGTACA
CCACCTTAAAAATTTTTTCGATCTGGCCCATTTGGTCGCCGCTCAGACCTTCATACGGGATGATGACATG
GATGTCGATCTTCAGCCCATTTTCACCGCTCAGGACAATCCTTTGGATCGGAGTTACGGACACCCCGAGA
TTCTGAAACAAACTGGACACACCTCCCTGTTCAAGGACTTGGTCCAGGTTGTAGCCGGCTGTCTGTCGCC
AGTCCCCAACGAAATCTTCGAGTGTGAAGACCATGGATCCGGGCCCGGGGTTTTCTTCAACGTCTCCAGC
CTGCTTCAGCAGGCTGAAGTTAGTAGCTCCGCTTCCTCGAGCTCGAGATCTGGCGAAGGCGATGGGGGTC
TTGAAGGCGTGCTGGTACTCCACGATGCCCAGCTCGGTGTTGCTGTGCAGCTCCTCCACGCGGCGGAAGG
CGAACATGGGGCCCCCGTTCTGCAGGATGCTGGGGTGGATGGCGCTCTTGAAGTGCATGTGGCTGTCCAC
CACGAAGCTGTAGTAGCCGCCGTCGCGCAGGCTGAAGGTGCGGGCGAAGCTGCCCACCAGCACGTTATCG
CCCATGGGGTGCAGGTGCTCCACGGTGGCGTTGCTGCGGATGATCTTGTCGGTGAAGATCACGCTGTCCT
CGGGGAAGCCGGTGCCCACCACCTTGAAGTCGCCGATCACGCGGCCGGCCTCGTAGCGGTAGCTGAAGCT
CACGTGCAGCACGCCGCCGTCCTCGTACTTCTCGATGCGGGTGTTGGTGTAGCCGCCGTTGTTGATGGCG
TGCAGGAAGGGGTTCTCGTAGCCGCTGGGGTAGGTGCCGAAGTGGTAGAAGCCGTAGCCCATCACGTGGC
TCAGCAGGTAGGGGCTGAAGGTCAGGGCGCCTTTGGTGCTCTTCATCTTGTTGGTCATGCGGCCCTGCTC
GGGGGTGCCCTCTCCGCCGCCCACCAGCTCGAACTCCACGCCGTTCAGGGTGCCGGTGATGCGGCACTCG
ATCTTCATGGCGGGCATGGTGGCGACCGGTAGCGCTAGCGGCTTCGGTACCACGCGTTCGCTCGAATTAA
TCAATTCTTTGCCAAAATGATGAGACAGCACAATAACCAGCACGTTGCCCAGGAGCTGTAGGAAAAAGAA
GAAGGCATGAACATGGTTAGCAGAGGCTCTAGAGCCGCCGGTCACACGCCAGAAGCCGAACCCCGCCCTG
CCCCGTCCCCCCCGAAGGCAGCCGTCCCCCCGCGGACAGCCCCGAGGCTGGAGAGGGAGAAGGGGACGGC
GGCGCGGCGACGCACGAAGGCCCTCCCCGCCCATTTCCTTCCTGCCGGGGCCCTCCCGGAGCCCCTCAAG
GCTTTCACGCAGCCACAGAAAAGAAACAAGCCGTCATTAAACCAAGCGCTAATTACAGCCCGGAGGAGAA
GGGCCGTCCCGCCCGCTCACCTGTGGGAGTAACGCGGTCAGTCAGAGCCGGGGCGGGCGGCGCGAGGCGG
CGCGGAGCGGGGCACGGGGCGAAGGCAACGCAGCGACTCCCGCCCGCCGCGCGCTTCGCTTTTTATAGGG
CCGCCGCCGCCGCCGCCTCGCCATAAAAGGAAACTTTCGGAGCGCGCCGCTCTGATTGGCTGCCGCCGCA
CCTCTCCGCCTCGCCCCGCCCCGCCCCTCGCCCCGCCCCGCCCCGCCTGGCGCGCGCCCCCCCCCCCCCC
CCGCCCCCATCGCTGCACAAAATAATTAAAAAATAAATAAATACAAAATTGGGGGTGGGGAGGGGGGGGA
GATGGGGAGAGTGAAGCAGAACGTGGGGCTCACCTCGACCATGGTAATAGCGATGACTAATACGTAGATG
TACTGCCAAGTAGGAAAGTCCCATAAGGTCATGTACTGGGCATAATGCCAGGCGGGCCATTTACCGTCAT
TGACGTCAATAGGGGGCGTACTTGGCATATGATACACTTGATGTACTGCCAAGTGGGCAGTTTACCGTAA
ATACTCCACCCATTGACGTCAATGGAAAGTCCCTATTGGCGTTACTATGGGAACATACGTCATTATTGAC
GTCAATGGGCGGGGGTCGTTGGGCGGTCAGCCAGGCGGGCCATTTACCGTAAGTTATGTAACGCGGAACA
ACAACAATTGCATTCATTTTATGTTTCAGGTTCAGGGGGAGGTGTGGTCCTGCGATTCCATCGAGTGCAC
CTACACCCTGCTGAAGACCCTATGCGGCCTAAGAGACCTGCTACCAATGAATTAAAAAAAAATGATTAAT
AAAAAATCACTTACTTGAAATCAGCAATAAGGTCTCTGTTGAAATTTTCTCCCAGCAGCACCTCACTTCC
CTCTTCCCAACTCTGGTATTCTAAACCCCGTTCAGCGGCATACTTTCTCCATACTTTAAAGGGGATGTCA
AATTTTAGCTCCTCTCCTGTACCCACAATCTTCATGTCTTTCTTCCCAGATGACCAAGAGAGTCCGGCTC
AGTGACTCCTTCAACCCTGTCTACCCCTATGAAGATGAAAGCACCTCCCAACACCCCTTTATAAACCCAG
GGTTTATTTCCCCAAATGGCTTCACACAAAGCCCAGACGGAGTTCTTACTTTAAAATGTTTAACCCCACT
AACAACCACAGGCGGATCTCTACAGCTAAAAGTGGGAGGGGGACTTACAGTGGATGACACTGATGGTACC
TTACAAGAAAACATACGTGCTACAGCACCCATTACTAAAAATAATCACTCTGTAGAACTATCCATTGGAA
ATGGATTAGAAACTCAAAACAATAAACTATGTGCCAAATTGGGAAATGGGTTAAAATTTAACAACGGTGA
CATTTGTATAAAGGATAGTATTAACACCTTATGGACTGGAATAAACCCTCCACCTAACTGTCAAATTGTG
GAAAACACTAATACAAATGATGGCAAACTTACTTTAGTATTAGTAAAAAATGGAGGGCTTGTTAATGGCT
ACGTGTCTCTAGTTGGTGTATCAGACACTGTGAACCAAATGTTCACACAAAAGACAGCAAACATCCAATT
AAGATTATATTTTGACTCTTCTGGAAATCTATTAACTGAGGAATCAGACTTAAAAATTCCACTTAAAAAT
AAATCTTCTACAGCGACCAGTGAAACTGTAGCCAGCAGCAAAGCCTTTATGCCAAGTACTACAGCTTATC
CCTTCAACACCACTACTAGGGATAGTGAAAACTACATTCATGGAATATGTTACTACATGACTAGTTATGA
TAGAAGTCTATTTCCCTTGAACATTTCTATAATGCTAAACAGCCGTATGATTTCTTCCAATGTTGCCTAT
GCCATACAATTTGAATGGAATCTAAATGCAAGTGAATCTCCAGAAAGCAACATAGCTACGCTGACCACAT
CCCCCTTTTTCTTTTCTTACATTACAGAAGACGACAACTAAAATAAAGTTTAAGTGTTTTTATTTAAAAT
CACAAAATTCGAGTAGTTATTTTGCCTCCACCTTCCCATTTGACAGAATACACAGTCCTTTCTCCCCGGC
TGGCCTTAAAAAGCATCATATCATGGGTAACAGACATATTCTTAGGTGTTATATTCCACACGGTTTCCTG
TCGAGCCAAACGCTCATCAGTGATATTAATAAACTCCCCGGGCAGCTCACTTAAGTTCATGTCGCTGTCC
AGCTGCTGAGCCACAGGCTGCTGTCCAACTTGCGGTTGCTTAACGGGCGGCGAAGGAGAAGTCCACGCCT
ACATGGGGGTAGAGTCATAATCGTGCATCAGGATAGGGCGGTGGTGCTGCAGCAGCGCGCGAATAAACTG
CTGCCGCCGCCGCTCCGTCCTGCAGGAATACAACATGGCAGTGGTCTCCTCAGCGATGATTCGCACCGCC
CGCAGCATAAGGCGCCTTGTCCTCCGGGCACAGCAGCGCACCCTGATCTCACTTAAATCAGCACAGTAAC
TGCAGCACAGCACCACAATATTGTTCAAAATCCCACAGTGCAAGGCGCTGTATCCAAAGCTCATGGCGGG
GACCACAGAACCCACGTGGCCATCATACCACAAGCGCAGGTAGATTAAGTGGCGACCCCTCATAAACACG
CTGGACATAAACATTACCTCTTTTGGCATGTTGTAATTCACCACCTCCCGGTACCATATAAACCTCTGAT
TAAACATGGCGCCATCCACCACCATCCTAAACCAGCTGGCCAAAACCTGCCCGCCGGCTATACACTGCAG
GGAACCGGGACTGGAACAATGACAGTGGAGAGCCCAGGACTCGTAACCATGGATCATCATGCTCGTCATG
ATATCAATGTTGGCACAACACAGGCACACGTGCATACACTTCCTCAGGATTACAAGCTCCTCCCGCGTTA
GAACCATATCCCAGGGAACAACCCATTCCTGAATCAGCGTAAATCCCACACTGCAGGGAAGACCTCGCAC
GTAACTCACGTTGTGCATTGTCAAAGTGTTACATTCGGGCAGCAGCGGATGATCCTCCAGTATGGTAGCG
CGGGTTTCTGTCTCAAAAGGAGGTAGACGATCCCTACTGTACGGAGTGCGCCGAGACAACCGAGATCGTG
TTGGTCGTAGTGTCATGCCAAATGGAACGCCGGACGTAGTCATTCTCGTATTTTGTATAGCAAAACGCGG
CCCTGGCAGAACACACTCTTCTTCGCCTTCTATCCTGCCGCTTAGCGTGTTCCGTGTGATAGTTCAAGTA
CAGCCACACTCTTAAGTTGGTCAAAAGAATGCTGGCTTCAGTTGTAATCAAAACTCCATCGCATCTAATT
GTTCTGAGGAAATCATCCACGGTAGCATATGCAAATCCCAACCAAGCAATGCAACTGGATTGCGTTTCAA
GCAGGAGAGGAGAGGGAAGAGACGGAAGAACCATGTTAATTTTTATTCCAAACGATCTCGCAGTACTTCA
AATTGTAGATCGCGCAGATGGCATCTCTCGCCCCCACTGTGTTGGTGAAAAAGCACAGCTAAATCAAAAG
AAATGCGATTTTCAAGGTGCTCAACGGTGGCTTCCAACAAAGCCTCCACGCGCACATCCAAGAACAAAAG
AATACCAAAAGAAGGAGCATTTTCTAACTCCTCAATCATCATATTACATTCCTGCACCATTCCCAGATAA
TTTTCAGCTTTCCAGCCTTGAATTATTCGTGTCAGTTCTTGTGGTAAATCCAATCCACACATTACAAACA
GGTCCCGGAGGGCGCCCTCCACCACCATTCTTAAACACACCCTCATAATGACAAAATATCTTGCTCCTGT
GTCACCTGTAGCGAATTGAGAATGGCAACATCAATTGACATGCCCTTGGCTCTAAGTTCTTCTTTAAGTT
CTAGTTGTAAAAACTCTCTCATATTATCACCAAACTGCTTAGCCAGAAGCCCCCCGGGAACAAGAGCAGG
GGACGCTACAGTGCAGTACAAGCGCAGACCTCCCCAATTGGCTCCAGCAAAAACAAGATTGGAATAAGCA
TATTGGGAACCACCAGTAATATCATCGAAGTTGCTGGAAATATAATCAGGCAGAGTTTCTTGTAGAAATT
GAATAAAAGAAAAATTTGCCAAAAAAACATTCAAAACCTCTGGGATGCAAATGCAATAGGTTACCGCGCT
GCGCTCCAACATTGTTAGTTTTGAATTAGTCTGCAAAAATAAAAAAAAAACAAGCGTCATATCATAGTAG
CCTGACGAACAGGTGGATAAATCAGTCTTTCCATCACAAGACAAGCCACAGGGTCTCCAGCTCGACCCTC
GTAAAACCTGTCATCGTGATTAAACAACAGCACCGAAAGTTCCTCGCGGTGACCAGCATGAATAAGTCTT
GATGAAGCATACAATCCAGACATGTTAGCATCAGTTAAGGAGAAAAAACAGCCAACATAGCCTTTGGGTA
TAATTATGCTTAATCGTAAGTATAGCAAAGCCACCCCTCGCGGATACAAAGTAAAAGGCACAGGAGAATA
AAAAATATAATTATTTCTCTGCTGCTGTTTAGGCAACGTCGCCCCCGGTCCCTCTAAATACACATACAAA
GCCTCATCAGCCATGGCTTACCAGAGAAAGTACAGCGGGCACACAAACCACAAGCTCTAAAGTCACTCTC
CAACCTCTCCACAATATATATACACAAGCCCTAAACTGACGTAATGGGACTAAAGTGTAAAAAATCCCGC
CAAACCCAACACACACCCCGAAACTGCGTCACCAGGGAAAAGTACAGTTTCACTTCCGCAATCCCAACAA
GCGTCACTTCCTCTTTCTCACGGTACGTCACATCCCATTAACTTACAACGTCATTTTCCCACGGCCGCGC
CGCCCCTTTTAACCGTTAACCCCACAGCCAATCACCACACGGCCCACACTTTTTAAAATCACCTCATTTA
CATATTGGCACCATTCCATCTATAAGGTATATTATTGATGATGGCCAAGCTATTTAGGTGACACTATAGA
ATACTCAAGCTATGCATCAAGCTTGGTACCGAGCTCGGATCCACTAGTAACGGCCGCCAGTGTGCTGGAA
TTCGCCCTTGTTTAAACGCGATCGCTTGAGATCGTTTTGGTCTGCGCGTAATCTCTTGCTCTGAAAACGA
AAAAACCGCCTTGCAGGGCGGTTTTTCGAAGGTTCTCTGAGCTACCAACTCTTTGAACCGAGGTAACTGG
CTTGGAGGAGCGCAGTCACCAAAACTTGTCCTTTCAGTTTAGCCTTAACCGGCGCATGACTTCAAGACTA
ACTCCTCTAAATCAATTACCAGTGGCTGCTGCCAGTGGTGCTTTTGCATGTCTTTCCGGGTTGGACTCAA
GACGATAGTTACCGGATAAGGCGCAGCGGTCGGACTGAACGGGGGGTTCGTGCATACAGTCCAGCTTGGA
GCGAACTGCCTACCCGGAACTGAGTGTCAGGCGTGGAATGAGACAAACGCGGCCATAACAGCGGAATGAC
ACCGGTAAACCGAAAGGCAGGAACAGGAGAGCGCACGAGGGAGCCGCCAGGGGGAAACGCCTGGTATCTT
TATAGTCCTGTCGGGTTTCGCCACCACTGATTTGAGCGTCAGATTTCGTGATGCTTGTCAGGGGGGCGGA
GCCTATGGAAAAACGGCTTTGCCGCGGCCCTCTCACTTCCCTGTTAAGTATCTTCCTGGCATCTTCCAGG
AAATCTCCGCCCCGTTCGTAAGCCATTTCCGCTCGCCGCAGTCGAACGACCGAGCGTAGCGAGTCAGTGA
GCGAGGAAGCGGAATATATCCTGTATCACATATTCTGCTGACGCACCGGTGCAGCCTTTTTTCTCCTGCC
ACATGAAGCACTTCACTGACACCCTCATCAGTGCCAACATAGTAAGCCAGTATACACTCCGCTAGCGCGA
TCGCTTAATTAATTTAAATCCTGCAGGGTTTAAACGGCCGGCCTAGGGATAACAGGGTAATCGTAACTAT
AACGGTCCTAAGGTAGCGAATGATGTCCGGCGGTGCTTTTGCCGTTACGCACCACCCCGTCAGTAGCTGA
ACAGGAGGGACAGCTGATAGAAACAGAAGCCACTGGAGCACCTCAAAAACACCATCATACACTAAATCAG
TAAGTTGGCAGCATCACCCGACGCACTTTGCGCCGAATAAATACCTGTGACGGAAGATCACTTCGCAGAA
TAAATAAATCCTGGTGTCCCTGTTGATACCGGGAAGCCCTGGGCCAACTTTTGGCGAAAATGAGACGTTG
ATCGGCACGTAAGAGGTTCCAACTTTCACCATAATGAAATAAGATCACTACCGGGCGTATTTTTTGAGTT
ATCGAGATTTTCAGGAGCTAAGGAAGCTAAAATGGAGAAAAAAATCACTGGATATACCACCGTTGATATA
TCCCAATGGCATCGTAAAGAACATTTTGAGGCATTTCAGTCAGTTGCTCAATGTACCTATAACCAGACCG
TTCAGCTGGATATTACGGCCTTTTTAAAGACCGTAAAGAAAAATAAGCACAAGTTTTATCCGGCCTTTAT
TCACATTCTTGCCCGCCTGATGAATGCTCATCCGGAATTCCGTATGGCAATGAAAGACGGTGAGCTGGTG
ATATGGGATAGTGTTCACCCTTGTTACACCGTTTTCCATGAGCAAACTGAAACGTTTTCATCGCTCTGGA
GTGAATACCACGACGATTTCCGGCAGTTTCTACACATATATTCGCAAGATGTGGCGTGTTACGGTGAAAA
CCTGGCCTATTTCCCTAAAGGGTTTATTGAGAATATGTTTTTCGTCTCAGCCAATCCCTGGGTGAGTTTC
ACCAGTTTTGATTTAAACGTGGCCAATATGGACAACTTCTTCGCCCCCGTTTTCACCATGGGCAAATATT
ATACGCAAGGCGACAAGGTGCTGATGCCGCTGGCGATTCAGGTTCATCATGCCGTCTGTGATGGCTTCCA
TGTCGGCAGAATGCTTAATGAATTACAACAGTACTGCGATGAGTGGCAGGGCGGGGCGTAACCTGCAGGT
TAATTAAGGAAGGGCGAATTCTGCAGATATCCATCACACTGGCGGCCGCTCGAGCATGCATCTAGAGGGC
CCAATTCGCCCTATAGTGAGTCGTATTACAATTCACTGGCC (SEQ ID NO: 177)
The nucleotide sequence of pHCA-Dual2-FI-globin-mgmtGFP:
GGCCGAAGGATTACATGAGCTTAGAAATGTAATTAGCATAGTGTGTGGCATAGTGTAGATACCAAATAAA
TATGATCTCTCCTTCTACTCTTGAAAATGCAAACACATTCTTGGTGGTCCTAAAATAGCCTGTAACATGG
TTTACTCAGCAGCATTTGCTATTCAAGGCAGATCTGCCTTTAGTCATTGGCTGCGCTCCTGAACAGCTGT
GTGAAAGGCTAACTTTTGTAAACCAAATCAAAATAAAATGCAGCAAAAATTTGTCACTGAAAGGAAATCC
TCAGTATATCCTTTTATGAAATGAAAGATCCCTCATCCAAACTTAACTTTTTTAAAAGTGCGCATTTGGA
GATATAGCCCTTTCTTATGAATCCTAATTCAATTTTGGCCATAAACACACGTTGATGTTCCCCACCCCAA
AGCACATAGCAACAAGAGTAGGTTCTATATTGAAAATAATGACAATTTAAAAACATGTACTTATTTCACT
GTATGTGGACAGTGTCTATGATTGCATCATGAAGTGTCATATAACCATGTACGTGTACATGAGAGAGAGA
TAGAGAGAGAAGTGGTAGGGTGGTGGTGGTAGAGGGGATGGCGATAGTAATCATGGTAATGGTAGAGGTG
ATGGAGGTGGTAATGACGGAGGTAAGGGTGGTAGTGATGATGGTGGTGGTGGTAATGGTGGTGGATGTGG
TGGTGGCAATTGGGATGGTGGGATGGTGGTAGCCATGGTGATGGTGGTAATGGTGTTGATTTAAAGGGTG
GTGGTAGTGAAGGTGAGGGTAGTGGTGGTGGAGGTGGTGGTGCTGGTAGCAATAGTGATGGTGGTGATGG
TGTTGATGAGGGTGTTGGGATCAGGGTGAGTTCCCACAGTATATTTCATTCTTGTTGTACCACTCTGTCA
ACAGCACCACTGACTGGGACAGAGGAAGAAGGCACACTCTGAATGTGTTATTAACAGAAACCTCAAAACA
GTCTGTCTCCTTGTAGTCATTCAAAATTATCTTTTTCTTACCTGGAAAACTGAAACTGAATTACCGGGAA
AAACACAGGAGATTTTTGTTTGTTAATATGCTGCCAATAAAGTAATTTTATGTCAAATTTAACTACAGGA
AAGGGCAAGGCATTTTCTAAGTTCCTTAGATGTCATGTGGCTAAAAAAAACAAAAGGATGGACAGCAGTT
AGATACTGTACACTTAGCTGTTTGAAGCCATATATTCAGAAAGCAGATGTTGGGAGTTGGTGTTTGAGGA
CTGATTTCCTGGAGGTATTTTATATAGGCCAAGTTCATTGTTCTAAACTCTAAGGGCTTGACTTGAGGGA
GGAAAAGAGGCAAGAACATGTTTAGTTTTGCTGACAGCATCACATGGGCAGCCCTAAGGCTAGACAACTT
TAGGGCCTGAAGCTTATTCTAGGAAAGAAGCACCTACAGAGTGGCACTGGGCTCCCCTCCACTATAGAGA
TGAAGTCATATGACAGTAAAGGGCAGGCAGGGCTGCCTAGGGGGCCCAGAACTGACACTTCCATTAGAAT
GAGCACAGGCCAGGGAGAGAAGTGGGGAACCAGAGAGAAGGAGCTGGAATTCCTTTCTCTCCATACATAA
ATGCCTGCAGAGTCCCATTTCAGAATCCGGCAGACAAAGCCACCAATGTGATCCCCATGACCTTATAAAC
ATTCATTAAAATGCATTTCAAGGCATGTGATGGCCTCCCCACCCCCTAGATAATGAGAAAACAAAGGTTT
CTCTTCTGATAGAGACAAGTTCAGCTCTGAAGTCAACATTATTTCTGGTTCTGTCTGAACAATGACATAT
GGCAACTCTTCCCTTTCTATAGTTCTAGTCCAGAATGACAAAAAAGGGGAAAAATTTCTTAGAGAAGGTA
GAGATTATACGAATACAGTCCATGAAATGAGCATAAGGAGAATAAAGAATATAACTTATCCAAAGAAGTC
TGGCAGGCTGTTATAAATGCTTGATTTTGGACACTGTAGTTGGAGGTTTAACATGGACACCAATAAAAAG
GTCAGCAAAGGGTATGCACTGTTCCTATTGGGCAAGAAGATAGGAGGTCAAAGGTAACCAGGAAAGATAA
ACTCAGGGAGACTTATTTTCCCTCCAGAGGGCACTGGGCTTGTAGGCCCTGGGCAAAATTGTCAAAAAGG
TGAAAATCGCCTGTGGTTTATTTAGTCTGCTCTTTCTTCACTAGTGCCTCACCAGTTCAGTTCAGGCCAA
TTTGCTAGCTACCACATTTGTAGAGGTTTTACTTGCTTTAAAAAACCTCCCACACCTCCCCCTGAACCTG
AAACATAAAATGAATGCAATTGTTGTTGTTAACTTGTTTATTGCAGCTTATAATGGTTACAAATAAAGCA
ATAGCATCACAAATTTCACAAATAAAGCATTTTTTTCACTGCATTCTAGTTGTGGTTTGTCCAAACTCAT
CAATGTATCTTATCATGTCTGAAGCTTTCTAGGTATTGAATAAGAAAAATGAAGTTAAGGTGGTTGATGG
TAACACTATGCTAATAACTGCAGAGCCAGAAGCACCATAAGGGACATGATAAGGGAGCCAGCAGACCTCT
GATCTCTTCCTGAATGCTAATCTTAAACATCCTGAGGAAGAATGGGACTTCCATTTGGGGTGGGCCTATG
ATAGGGTAATAAGACAGTAGTGAATATCAAGCTACAAAAAGCCCCCTTTCAAATTCTTCTCAGTCCTAAC
TTTTCATACTAAGCCCAGTCCTTCCAAAGCAGACTGTGAAAGAGTGATAGTTCCGGGAGACTAGCACTGC
AGATTCCGGGTCACTGTGAGTGGGGGAGGCAGGGAAGAAGGGCTCACAGGACAGTCAAACCATGCCCCCT
GTTTTTCCTTCTTCAAGTAGACCTCTATAAGACAACAGAGACAACTAAGGCTGAGTGGCCAGGCGAGGAG
AAACCATCTCGCCGTAAAACATGGAAGGAACACTTCAGGGGAAAGGTGGTATCTCTAAGCAAGAGAACTG
AGTGGAGTCAAGGCTGAGAGATGCAGGATAAGCAAATGGGTAGTGAAAAGACATTCATGAGGACAGCTAA
AACAATAAGTAATGTAAAATACAGCATAGCAAAACTTTAACCTCCAAATCAAGCCTCTACTTGAATCCTT
TTCTGAGGGATGAATAAGGCATAGGCATCAGGGGCTGTTGCCAATGTGCATTAGCTGTTTGCAGCCTCAC
CTTCTTTCATGGAGTTTAAGATATAGTGTATTTTCCCAAGGTTTGAACTAGCTCTTCATTTCTTTATGTT
TTAAATGCACTGACCTCCCACATTCCCTTTTTAGTAAAATATTCAGAAATAATTTAAATACATCATTGCA
ATGAAAATAAATGTTTTTTATTAGGCAGAATCCAGATGCTCAAGGCCCTTCATAATATCCCCCAGTTTAG
TAGTTGGACTTAGGGAACAAAGGAACCTTTAATAGAAATTGGACAGCAAGAAAGCGAGCTTAGTGATACT
TGTGGGCCAGGGCATTAGCCACACCAGCCACCACTTTCTGATAGGCAGCCTGCACTGGTGGGGTGGCGGC
CGCCCTAGGATTATGGCACTGGTAGAATTCACTACTTATGGCACTGGTAGAATTCACTACTTATGGCACT
GGTAGAATTCACTACTTATGGCACTGGTAGAATTCACTATCGTTGTGCTTGATCTAACCATGTTTCATTG
TGCTTGATCTAACCATGTTTCATTGTGCTTGATCTAACCATGTTTCATTGTGCTTGATCTAACCATGTAT
CGCCCGGGGGCGGCCGCACACAAAAAACCAACACACAGATCTAATGAAAATAAAGATCTTTTATTGAATT
CTTAGCTGGCCTCCACCTTTCTCTTCTTCTTGGGGCTGTCGCCTCCCAGCTGAGACAGGTCGATCCGTGT
CTCGTACAGGCCGGTGATGCTCTGGTGGATCAGGGTGGCGTCCAGCACCTCTTTGGTGCTGGTGTACCTC
TTCCGGTCGATGGTGGTGTCAAAGTACTTGAAGGCGGCAGGGGCTCCCAGATTGGTCAGGGTAAACAGGT
GGATGATATTCTCGGCCTGCTCTCTGATGGGCTTATCCCGGTGCTTGTTGTAGGCGGACAGCACTTTGTC
CAGATTAGCGTCGGCCAGGATCACTCTCTTGGAGAACTCGCTGATCTGCTCGATGATCTCGTCCAGGTAG
TGCTTGTGCTGTTCCACAAACAGCTGTTTCTGCTCATTATCCTCGGGGGAGCCCTTCAGCTTCTCATAGT
GGCTGGCCAGGTACAGGAAGTTCACATATTTGGAGGGCAGGGCCAGTTCGTTTCCCTTCTGCAGTTCGCC
GGCAGAGGCCAGCATTCTCTTCCGGCCGTTTTCCAGCTCGAACAGGGAGTACTTAGGCAGCTTGATGATC
AGGTCCTTTTTCACTTCTTTGTAGCCCTTGGCTTCCAGAAAGTCGATGGGATTCTTCTCGAAGCTGCTTC
TTTCCATGATGGTGATCCCCAGCAGCTCTTTCACACTCTTCAGTTTCTTGGACTTGCCCTTTTCCACTTT
GGCCACCACCAGCACAGAATAGGCCACGGTGGGGCTGTCGAAGCCGCCGTACTTCTTAGGGTCCCAGTCC
TTCTTTCTGGCGATCAGCTTATCGCTGTTCCTCTTGGGCAGGATAGACTCTTTGCTGAAGCCGCCTGTCT
GCACCTCGGTCTTTTTCACGATATTCACTTGGGGCATGCTCAGCACTTTCCGCACGGTGGCAAAATCCCG
GCCCTTATCCCACACGATCTCCCCGGTTTCGCCGTTTGTCTCGATCAGAGGCCGCTTCCGGATCTCGCCG
TTGGCCAGGGTAATCTCGGTCTTGAAAAAGTTCATGATGTTGCTGTAGAAGAAGTACTTGGCGGTAGCCT
TGCCGATTTCCTGCTCGCTCTTGGCGATCATCTTCCGCACGTCGTACACCTTGTAGTCGCCGTACACGAA
CTCGCTTTCCAGCTTAGGGTACTTTTTGATCAGGGCGGTTCCCACGACGGCGTTCAGGTAGGCGTCGTGG
GCGTGGTGGTAGTTGTTGATCTCGCGCACTTTGTAAAACTGGAAATCCTTCCGGAAATCGGACACCAGCT
TGGACTTCAGGGTGATCACTTTCACTTCCCGGATCAGCTTGTCATTCTCGTCGTACTTAGTGTTCATCCG
GGAGTCCAGGATCTGTGCCACGTGCTTTGTGATCTGCCGGGTTTCCACCAGCTGTCTCTTGATGAAGCCG
GCCTTATCCAGTTCGCTCAGGCCGCCTCTCTCGGCCTTGGTCAGATTGTCGAACTTTCTCTGGGTAATCA
GCTTGGCGTTCAGCAGCTGCCGCCAGTAGTTCTTCATCTTCTTCACGACCTCTTCGGAGGGCACGTTGTC
GCTCTTGCCCCGGTTCTTGTCGCTTCTGGTCAGCACCTTGTTGTCGATGGAGTCGTCCTTCAGAAAGCTC
TGAGGCACGATATGGTCCACATCGTAGTCGGACAGCCGGTTGATGTCCAGTTCCTGGTCCACGTACATAT
CCCGCCCATTCTGCAGGTAGTACAGGTACAGCTTCTCGTTCTGCAGCTGGGTGTTTTCCACGGGGTGTTC
TTTCAGGATCTGGCTGCCCAGCTCTTTGATGCCCTCTTCGATCCGCTTCATTCTCTCGCGGCTGTTCTTC
TGTCCCTTCTGGGTGGTCTGGTTCTCTCTGGCCATTTCGATCACGATGTTCTCGGGCTTGTGCCGGCCCA
TCACTTTCACGAGCTCGTCCACCACCTTCACTGTCTGCAGGATGCCCTTCTTAATGGCGGGGCTGCCGGC
CAGATTGGCAATGTGCTCGTGCAGGCTATCGCCCTGGCCGGACACCTGGGCTTTCTGGATGTCCTCTTTA
AAGGTCAGGCTGTCGTCGTGGATCAGCTGCATGAAGTTTCTGTTGGCGAAGCCGTCGGACTTCAGGAAAT
CCAGGATTGTCTTGCCGGACTGCTTGTCCCGGATGCCGTTGATCAGCTTCCGGCTCAGCCTGCCCCAGCC
GGTGTATCTCCGCCGCTTCAGCTGCTTCATCACTTTGTCGTCGAACAGGTGGGCATAGGTTTTCAGCCGT
TCCTCGATCATCTCTCTGTCCTCAAACAGTGTCAGGGTCAGCACGATATCTTCCAGAATGTCCTCGTTTT
CCTCATTGTCCAGGAAGTCCTTGTCCTTGATAATTTTCAGCAGATCGTGGTATGTGCCCAGGGAGGCGTT
GAACCGATCTTCCACGCCGGAGATTTCCACGGAGTCGAAGCACTCGATTTTCTTGAAGTAGTCCTCTTTC
AGCTGCTTCACGGTCACTTTCCGGTTGGTCTTGAACAGCAGGTCCACGATGGCCTTTTTCTGCTCGCCGC
TCAGGAAGGCGGGCTTTCTCATTCCCTCGGTCACGTATTTCACTTTGGTCAGCTCGTTATACACGGTGAA
GTACTCGTACAGCAGGCTGTGCTTGGGCAGCACCTTCTCGTTGGGCAGGTTCTTATCGAAGTTGGTCATC
CGCTCGATGAAGCTCTGGGCGGAAGCGCCCTTGTCCACCACTTCCTCGAAGTTCCAGGGGGTGATGGTTT
CCTCGCTCTTTCTGGTCATCCAGGCGAATCTGCTGTTTCCCCTGGCCAGAGGGCCCACGTAGTAGGGGAT
GCGGAAGGTCAGGATCTTCTCGATCTTTTCCCGGTTGTCCTTCAGGAATGGGTAAAAATCTTCCTGCCGC
CGCAGAATGGCGTGCAGCTCTCCCAGGTGGATCTGGTGGGGGATGCTGCCGTTGTCGAAGGTCCGCTGCT
TCCGCAGCAGGTCCTCTCTGTTCAGCTTCACGAGCAGTTCCTCGGTGCCGTCCATCTTTTCCAGGATGGG
CTTGATGAACTTGTAGAACTCTTCCTGGCTGGCTCCGCCGTCAATGTAGCCGGCGTAGCCGTTCTTGCTC
TGGTCGAAGAAAATCTCTTTGTACTTCTCAGGCAGCTGCTGCCGCACGAGAGCTTTCAGCAGGGTCAGGT
CCTGGTGGTGCTCGTCGTATCTCTTGATCATAGAGGCGCTCAGGGGGGCCTTGGTGATCTCGGTGTTCAC
TCTCAGGATGTCGCTCAGCAGGATGGCGTCGGACAGGTTCTTGGCGGCCAGAAACAGGTCGGCGTACTGG
TCGCCGATCTGGGCCAGCAGGTTGTCCAGGTCGTCGTCGTAGGTGTCCTTGCTCAGCTGCAGTTTGGCAT
CCTCGGCCAGGTCGAAGTTGCTCTTGAAGTTGGGGGTCAGGCCCAGGCTCAGGGCAATCAGGTTGCCGAA
CAGGCCATTCTTCTTCTCGCCGGGCAGCTGGGCGATCAGATTTTCCAGCCGTCTGCTCTTGCTCAGTCTG
GCAGACAGGATGGCCTTGGCGTCCACGCCGCTGGCGTTGATGGGGTTTTCCTCGAACAGCTGGTTGTAGG
TCTGCACCAGCTGGATGAACAGCTTGTCCACGTCGCTGTTGTCGGGGTTCAGGTCGCCCTCGATCAGGAA
GTGGCCCCGGAACTTGATCATGTGGGCCAGGGCCAGATAGATCAGCCGCAGGTCGGCCTTGTCGGTGCTG
TCCACCAGTTTCTTTCTCAGGTGGTAGATGGTGGGGTACTTCTCGTGGTAGGCCACCTCGTCCACGATGT
TGCCGAAGATGGGGTGCCGCTCGTGCTTCTTATCCTCTTCCACCAGGAAGGACTCTTCCAGTCTGTGGAA
GAAGCTGTCGTCCACCTTGGCCATCTCGTTGCTGAAGATCTCTTGCAGATAGCAGATCCGGTTCTTCCGT
CTGGTGTATCTTCTTCTGGCGGTTCTCTTCAGCCGGGTGGCCTCGGCTGTTTCGCCGCTGTCGAACAGCA
GGGCTCCGATCAGGTTCTTCTTGATGCTGTGCCGGTCGGTGTTGCCCAGCACCTTGAATTTCTTGCTGGG
CACCTTGTACTCGTCGGTGATCACGGCCCAGCCCACAGAGTTGGTGCCGATGTCCAGGCCGATGCTGTAC
TTCTTGTCGGACGCTTCGACCTTGCGCTTTTTCTTCGGCGAAGCGTAATCTGGAACATCGTATGGGTACA
TGGTGGCCCTAGGATCGATTACTAGCTCACGACACCTGAAATGGAAGAAAAAAACTTTGAACCACTGTCT
GAGGCTTGAGAATGAACCAAGATCCAAACTCAAAAAGGGCAAATTCCAAGGAGAATTACATCAAGTGCCA
AGCTGGCCTAACTTCAGTCTCCACCCACTCAGTGTGGGGAAACTCCATCGCATAAAACCCCTCCCCCCAA
CCTAAAGACGACGTACTCCAAAAGCTCGAGAACTAATCGAGGTGCCTGGACGGCGCCCGGTACTCCGTGG
AGTCACATGAAGCGACGGCTGAGGACGGAAAGGCCCTTTTCCTTTGTGTGGGTGACTCACCCGCCCGCTC
TCCCGAGCGCCGCGTCCTCCATTTTGAGCTCCCTGCAGCAGGGCCGGGAAGCGGCCATCTTTCCGCTCAC
GCAACTGGTGCCGACCGGGCCAGCCTTGCCGCCCAGGGCGGGGCGATACACGGCGGCGCGAGGCCAGGCA
CCAGAGCAGGCCGGCGAGCTTGAGACTACCCCCGTCCGATTCTCGGTGGCCGCGCTCGCAGGCCCCGCCT
CGCCGAACATGTGCGCTGGGACGCACGGGCCCCGTCGCCGCCCGCGGCCCCAAAAACCGAAATACCAGTG
TGCAGATCTTGGCCCGCATTTACAAGACTATCTTGCCAGAAAAAAAGCGTCGCAGCAGGTCATCAAAAAT
TTTAAATGGCTAGAGACTTATCGAAAGCAGCGAGACAGGCGCGAAGGTGCCACCAGATTCGCACGCGGCG
GCCCCAGCGCCCAGGCCAGGCCTCAACTCAAGCACGAGGCGAAGGGGCTCCTTAAGCGCAAGGCCTCGAA
CTCTCCCACCCACTTCCAACCCGAAGCTCGGGATCAAGAATCACGTACTGCAGCCAGGGGCGTGGAAGTA
ATTCAAGGCACGCAAGGGCCATAACCCGTAAAGAGGCCAGGCCCGCGGGAACCACACACGGCACTTACCT
GTGTTCTGGCGGCAAACCCGTTGCGAAAAAGAACGTTCACGGCGACTACTGCACTTATATACGGTTCTCC
CCCACCCTCGGGAAAAAGGCGGAGCCAGTACACGACATCACTTTCCCAGTTTACCCCGCGCCACCTTCTC
TAGGCACCGGTTCAATTGCCGACCCCTCCCCCCAACTTCTCGGGGACTGTGGGCGATGTGCGCTCTGCCC
ACTGACGGGCACCGGAGCCTCACGCATGCTCTTCTCCACCTCAGTGATGACGAGAGCGGGCGGGTGAGGG
GGCGGGAACGCAGCGATCTCTGGGTTCTACGTTAGTGGGAGTTTAACGACGGTCCCTGGGATTCCCCAAG
GCAGGGGCGAGTCCTTTTGTATGAATTACTCAAATCGATTAGGATCCGGCGCGCCCACCGCGGAAAAAAA
GCACCGACTCGGTGCCACTTTTTCAAGTTGATAACGGACTAGCCTTATTTTAACTTGCTATTTCTAGCTC
TAAAACGTGATAAAAGCAACTGTTAGCGGTGTTTCGTCCTTTCCACAAGATATATAAAGCCAAGAAATCG
AAATACTTTCAAGTTACGATAAGCATATGATAGTCCATTTTAAAACATAATTTTAAAACTGCAAACTACC
CAAGAAATTATTACTTTCTACGTCACGTATTTTGTACTAATATCTTTGTGTTTACAGTCAAATTAATTCC
AATTATCTCTCTAACAGCCTTGTATCGTATATGCAAATATGAAGGAATCATGGGAAATAGGCCCTCGGCG
CGCCCACCGCGGAAAAAAAGCACCGACTCGGTGCCACTTTTTCAAGTTGATAACGGACTAGCCTTATTTT
AACTTGCTATTTCTAGCTCTAAAACTGACCAATAGCCTTGACAAGCGGTGTTTCGTCCTTTCCACAAGAT
ATATAAAGCCAAGAAATCGAAATACTTTCAAGTTACGGTAAGCATATGATAGTCCATTTTAAAACATAAT
TTTAAAACTGCAAACTACCCAAGAAATTATTACTTTCTACGTCACGTATTTTGTACTAATATCTTTGTGT
TTACAGTCAAATTAATTCTAATTATCTCTCTAACAGCCTTGTATCGTATATGCAAATATGAAGGAATCAT
GGGAAATAGGCCCTCGGATCCTTAATTGCTAGCGAGGCCTGTACTCTGTTTTCACAGGAAGAAATCCTCA
CCCAGTCTTCCCCAAACACATTCCCAGGTTCTGTCATTAGTGGGATAGAGATGATTACTGTGGGGAGAAG
AGAAACATCTGGATGGATTTGGTGAGGTTGATCTATAGAGGAAGTAGGTGCTGCCTGAGGTAGCTGTAAT
AGAAGCTAAAGGTCAAAGGAGAGGGCCCTGTCCCAATCCAGATGACTCCACTTCTGCTGGACCCAGGTTC
ACAAGCTTAATCTACATTTCACCTAAATTTGGCTAACAAGCCCAAAATCACACAGGCAAAGGGAGAAGTG
GAGGCAGAACCGAGGTTGGAGGCCACCAGGGCCACCGGGCAGAGATCATTTAAGCCCAACCTTCTCACTT
CTCCCTGGGCTCTGCCTCTCTTAAAGGACCTTGTGGTGTGACCTCTTGTAGGTCCCTTTCACACTCGGGG
CCTCAGTTTCCCCACTGTAAAGTGAATGGGTCCCAGCTTTGGTAAGCTTATGCTTACCTGATGCTTTCTT
CCTGGGCTGCTCTTGTAGAGAAAAGATAAATCTTCTTCCTCCATCCACGAGGGCTTCTTTCCCTGGGGGT
GAGAGTAGGCTGAGGAGAGCCACTTGCACACACTCTTAAAGAAAGTATTACCTGCACCAGCTCAGTGAGA
GGCACAGATCAGACTGTTACTTGAATCAAATTATGAGCCTCCCCAAATATATCTATGACATTTAAATAGG
GGATTACTTGAACATAGACTTTGGGATCCGGTGTGGAGTGCAGGAGACTAGCAAAGTGAATCCTGAGAGT
AGCAGGTCTGCACCTGTTGGATCGAGAAAGGCGGCCTACAATTCTGGTCAAATGAGCTGTGCTTATTGAC
ATATTCTATTAGAGAGTACTACCAGGTCACCAGTCACCAGAAAGGCTGCCAGCTCTCCAACCACCTCCAG
GGAACTATCCTGAATGGGGCCTTAACAAGCCTAAGAGAGGGTTGGTTTGGGTCCCAAGCCAATATTTGCT
CTGCTTTATGTCAGTCATATGGAACCCAAACCAACCCTCTCCTATGTGCCTCACCAGTCGGTGCAGGGAT
CCCAATTTCAAGTTTGGTTTTTTATGGTCAAAGTCCAGCATAGATTAAATGAAGGGGTGTGATGATGGTG
TTAAAAGAGAACTCCAGACCAGTTTAACTCTTGGACACACATCCCATCTCACCATGGTGCTTCCAACCTT
CCAGAGATGATGGGCTCCTATTTTCTGATGACAAAGCCCTCCACAGGATTGCTGCCTGGCCATCAGGGAG
TGCCTCTGTAACTGAGGCTGAGATCCCACTTTCAGTCCTCCAGCTGTGGCCCATCCCTGCTCCGCCCACC
GGGTATGGCCTGTCCTAGGCTCTTAGGTATGGCTGCATTGTGAAATGATGGCTACAGAGCTGGCATCTCC
TGTAGTCTGGTTCATCTAGTGCACTACCTCATAGTTAAAAGAAATCTGTTTAAGCCACTGAGGGTGGCTC
CTAGTGCCAACTCCAAGAACAGGAAGCTTCCCTTTTTTGGGAGGAGGGGCAGATGGTAACATGGATCGTC
CAGGTCAATGGGAGCAGGGCAACCACAGTAAGTACTGGACAACAACACAAAACTCCATGTGTGGCTTCCA
TCGAGTCCCTCTCCAATTGGTTTGGTCTTCTCCGTCCCATGCAGCACTTTAGCAAGGGGCCTGGCTGAAG
GCTATGAATTGTGTGGAGCCTCCTCATTGCAGTCTCCAACCATCTGATGCTGGGAAAATGTCACCAGGAT
GCAGCCATGCCGTGTGGCCAATGAACCGAGAAAACACCCCTTTTCTAGAATGCTCTAAAGAGGCAGAATA
ATCCAGAGGTGAGGAAGGAAATACTCCACCAGAGACCCAGGCAGTTCCTACAAAAGCCAGACTTTCCTTC
ACCTAGGGAGTGACAAGACCAGTGGAAAACACTCTCAAGCAGTAACCCCCAAATGCTCTGCAAGCCAGTG
GCGTCCAGATACCGCACAAGCGAGTGGGCTGTCTAATCCCATCATCATGATGTAAATATCTCTAGGCTGC
CCCGGGCTGTGCCTGACCCTGTCTTCAGCTTTCCACACCTCCACCTACAGCCCATGCACAGAAGGACCAC
CCAGGAATGCTGCAAGTGTGGCACCTCCAGGGCCACCCAGGGAGAAGGAGGGCAGCTATGCTGGTGGCTC
CAGGCCCATTTGGCGGGTGGTACCTTCACACCACAAAGCCCAAACTGAGGCCCCAGATTTGGCTGATGAG
GGCATATTGGACAGGGGTCACTTATGCTCTTCCCCATTGCCACCTGGCCTCTGGCTACCTGGACTTGGCT
ACCTGTGGATCCTCTCACAGGTGCCACCATCTTGGCTGAGTCTCCAGATGCGAGGTCCCTGAGGCAGTGG
CAGGCTTCTCGCTAATGCTGATGGGATTAGGAATGGGATAGGTGGGGAGGGCCCTGGACTGGGCCCTGAT
GAGCCAAGTGGGTTTTTAGAGGGGCTACTGGTACATTTCAGGGACAGGACATCTGGTAGAGCTAAGCTGG
GGCAATAAGGAGCCACTGCTAATCTGAGAGCTAGAAACAATCAGCTTCTGGGTCATTATTAATTAGGGTA
GTTTGGGCTGTGTGGAAGTCACGTACTATATGGGGTAGCCACAGCTCTCTCTACAGATAATCTCTAAGAC
TTCTGATTGGGACCGTGTGAATGCAGTAGCAATATCTCTTCTTACTGCCAGGCCCTGCCAGTCCTGCCTC
CACGCCCTGGCTGGCCCCCCTTATGATCTGACCCATGCCAGGCTGCCATAGTATGTTACTTCTGCATTAG
CACTCCTTGGGACCTGCCTCTCCACTGTCCCTCAGACTTTAAAGAACTATACAAACCCAAGGGGCTCTTC
CCAAGAGAATTGATATGACTTGAGGTGATTCCATTTCTGGAAGTAGTCACTCCATTTTCTGCCTCACTCT
TTCAGTGCTTCACAGAGCAGGTTCGATTAAGCACACAGATTAATTAAGCACACAGATTAATCGTAACTAT
AACGGTCCTAAGGTAGCGAAAGCTTGCATGCCTGCAGGTCGACTCTAGAGGATCAAACCTAGCCACCGCG
GTGGCGGCCGGCTAGCCGGCTAGCCGGCTAGCCCTAGAACTAGTAACGGCCGCCAGTGTGCTGGAATTCG
GCTTGTAAGGTACCGGTGAAGTTCCTATACTTTCTAGAGAATAGGAACTTCGGAATAGGAACTTCTACCT
AGATGCATGCTCGAGCGGCCCCTACAGTTGAAGTCGGAAGTTTACATACACTTAAGTTGGAGTCATTAAA
ACTCGTTTTTCAACTACTCCACAAATTTCTTGTTAACAAACAATAGTTTTGGCAAGTCAGTTAGGACATC
TACTTTGTGCATGACACAAGTCATTTTTCCAACAATTGTTTACAGACAGATTATTTCACTTATAATTCAC
TGTATCACAATTCCAGTGGGTCAGAAGTTTACATACACTAAGTTGACTGTGCCTTTAAACAGCTTGGAAA
ATTCCAGAAAATGATGTCATGGCTTTAGAAGCTTCTGATAGACTAATTGACATCATTTGAGTCAATTGGA
GGTGTACCTGTGGATGTATTTCAAGGAATTCTGTGGAATGTGTGTCAGTTAGGGTGTGGAAAGTCCCCAG
GCTCCCCAGCAGGCAGAAGTATGCAAAGCATGCATATCGATACTAGTTTATAAGATCTCGAGCTAGGGTA
CCGTCAAGGCTGCAGTGAGACATGATCTTGCCACTGCACTCCAGCCTGGACAGCAGAGTGAAACCTTGCC
TCACGAAACAGAATACAAAAACAAACAAACAAAAAACTGCTCCGCAATGCGCTTCCTTGATGCTCTACCA
CATAGGTCTGGGTACTTTGTACACATTATCTCATTGCTGTTCATAATTGTTAGATTAATTTTGTAATATT
GATATTATTCCTAGAAAGCTGAGGCCTCAAGATGATAACTTTTATTTTCTGGACTTGTAATAGCTTTCTC
TTGTATTCACCATGTTGTAACTTTCTTAGAGTAGTAACAATATAAAGTTATTGTGAGTTTTTGCAAACAC
AGCAAACACAACGACCCATATAGACATTGATGTGAAATTGTCTATTGTCAATTTATGGGAAAACAAGTAT
GTACTTTTTCTACTAAGCCATTGAAACAGGAATAACAGAACAAGATTGAAAGAATACATTTTCCGAAATT
ACTTGAGTATTATACAAAGACAAGCACGTGGACCTGGGAGGAGGGTTATTGTCCATGACTGGTGTGTGGA
GACAAATGCAGGTTTATAATAGATGGGATGGCATCTAGCGCAATGACTTTGCCATCACTTTTAGAGAGCT
CTTGGGGACCCCAGTACACAAGAGGGGACGCAGGGTATATGTAGACATCTCATTCTTTTTCTTAGTGTGA
GAATAAGAATAGCCATGACCTGAGTTTATAGACAATGAGCCCTTTTCTCTCTCCCACTCAGCAGCTATGA
GATGGCTTGCCCTGCCTCTCTACTAGGCTGACTCACTCCAAGGCCCAGCAATGGGCAGGGCTCTGTCAGG
GCTTTGATAGCACTATCTGCAGAGCCAGGGCCGAGAAGGGGTGGACTCCAGAGACTCTCCCTCCCATTCC
CGAGCAGGGTTTGCTTATTTATGCATTTAAATGATATATTTATTTTAAAAGAAATAACAGGAGACTGCCC
AGCCCTGGCTGTGACATGGAAACTATGTAGAATATTTTGGGTTCCATTTTTTTTTCCTTCTTTCAGTTAG
AGGAAAAGGGGCTCAGGATCCACTTGCCCAGTGTTCTTCCTTAGTTCCTACCTTCGACCTTGATCCTCCT
TTATCTTCCTGAACCCTGCTGAGATGATCTATGTGGGGAGAATGGCTTCTTTGAGAAACATCTTCTTCGT
TAGTGGCCTGCCCCTCATTCCCACTTTAATATCCAGAATCACTATAAGAAGAATATAATAAGAGGAATAA
CTCTTATTATAGGTAAGGGAAAATTAAGAGGCATACGTGATGGGATGAGTAAGAGAGGAGAGGGAAGGAT
TAATGGACGATAAAATCTACTACTATTTGTTGAGACCTTTTATAGTCTAATCAATTTTGCTATTGTTTTC
CATCCTCACGCTAACTCCATAAAAAAACACTATTATTATCTTTATTTTGCCATGACAAGACTGAGCTCAG
AAGAGTCAAGCATTTGCCTAAGGTCGGACATGTCAGAGGCAGTGCCAGACCTATGTGAGACTCTGCAGCT
ACTGCTCATGGGCCCTGTGCTGCACTGATGAGGAGGATCAGATGGATGGGGCAATGAAGCAAAGGAATCA
TTCTGTGGATAAAGGAGACAGCCATGAAGAAGTCTATGACTGTAAATTTGGGAGCAGGAGTCTCTAAGGA
CTTGGATTTCAAGGAATTTTGACTCAGCAAACACAAGACCCTCACGGTGACTTTGCGAGCTGGTGTGCCA
GATGTGTCTATCAGAGGTTCCAGGGAGGGTGGGGTGGGGTCAGGGCTGGCCACCAGCTATCAGGGCCCAG
ATGGGTTATAGGCTGGCAGGCTCAGATAGGTGGTTAGGTCAGGTTGGTGGTGCTGGGTGGAGTCCATGAC
TCCCAGGAGCCAGGAGAGATAGACCATGAGTAGAGGGCAGACATGGGAAAGGTGGGGGAGGCACAGCATA
GCAGCATTTTTCATTCTACTACTACATGGGACTGCTCCCCTATACCCCCAGCTAGGGGCAAGTGCCTTGA
CTCCTATGTTTTCAGGATCATCATCTATAAAGTAAGAGTAATAATTGTGTCTATCTCATAGGGTTATTAT
GAGGATCAAAGGAGATGCACACTCTCTGGACCAGTGGCCTAACAGTTCAGGACAGAGCTATGGGCTTCCT
ATGTATGGGTCAGTGGTCTCAATGTAGCAGGCAAGTTCCAGAAGATAGCATCAACCACTGTTAGAGATAT
ACTGCCAGTCTCAGAGCCTGATGTTAATTTAGCAATGGGCTGGGACCCTCCTCCAGTAGAACCTTCTAAC
CAGCTGCTGCAGTCAAAGTCGAATGCAGCTGGTTAGACTTTTTTTAATGAAAGCTTGCATGCAGCACTTT
GGGAGGCTGAGGTGGGTGGACTGCTTGGAGCTCAGGAGTTCAAGACCATCTTGGACAACATGGTGATACC
CTGCCTCTACAAAAAGTACAAAAATTAGCCTGGCATGGTGGTGTGCACCTGTAATCCCAGCTATTAGGGT
GGCTGAGGCAGGAGAATTGCTTGAACCCGGGAGGCGGAGGTTGCAGTGAGCTGAGATCGTGCCACTGCAC
TCCAGCCTGGGGGACAGAGCACATTATAATTAACTGTTATTTTTTACTTGGACTCTTGTGGGGAATAAGA
TACATGTTTTATTCTTATTTATGATTCAAGCACTGAAAATAGTGTTTAGCATCCAGCAGGTGCTTCAAAA
CCATTTGCTGAATGATTACTATACTTTTTACAAGCTCAGCTCCCTCTATCCCTTCCAGCATCCTCATCTC
TGATTAAATAAGCTTCAGTTTTTCCTTAGTTCCTGTTACATTTCTGTGTGTCTCCATTAGTGACCTCCCA
TAGTCCAAGCATGAGCAGTTCTGGCCAGGCCCCTGTCGGGGTCAGTGCCCCACCCCCGCCTTCTGGTTCT
GTGTAACCTTCTAAGCAAACCTTCTGGCTCAAGCACAGCAATGCTGAGTCATGATGAGTCATGCTGAGGC
TTAGGGTGTGTGCCCAGATGTTCTCAGCCTAGAGTGATGACTCCTATCTGGGTCCCCAGCAGGATGCTTA
CAGGGCAGATGGCAAAAAAAAGGAGAAGCTGACCACCTGACTAAAACTCCACCTCAAACGGCATCATAAA
GAAAATGGATGCCTGAGACAGAATGTGACATATTCTAGAATATATTATTTCCTGAATATATATATATATA
TACACATATACCATATGAAACACCTCTAGGCTATAAGGCAACAGAGCTCCTTTTTTTTTTTTCTGTGCTT
TCCTGGCTGTCCAAATCTCTAATGATAAGCATACTTCTATTCAATGAGAATATTCTGTAAGATTATAGTT
AAGAATTGTGGGAGCCATTCCGTCTCTTATAGTTAAATTTGAGCTTCTTTTATGATCACTGTTTTTTTAA
TATGCTTTAAGTTCTGGGGTACATGTGCCATGGTGGTTTGCTGCACCCATCAACCCGTCATCTACATTAG
GTATTTCTCCTAATGCTATCCTTCCCCTAGCCCCCCACCCCCAACAGGCCCCAGTGTGTGATGTTCCCCT
CCCTGTGTCCATGGATCACTGGTTTTTTTTTGTTTTTTTTTTTTTTTTAAAGTCTCAGTTAAATTTTTGG
AATGTAATTTATTTTCCTGGTATCCTAAGGACTTGCAAGTTATCTGGTCACTTTAGCCCTCACGTTTTGA
TGATAATCACATATTTGTAAACACAACACACACACACACACACACACACATATATATATATATAAAACAT
ATATATACATAAACACACATAACATATTTATCGGGCATTTCTGAGCAACTAATCATGCAGGACTCTCAAA
CACTAACCTATAGCCTTTTCTATGTATCTACTTGTGTAGAAACCAAGCGTGGGGACTGAGAAGGCAATAG
CAGGAGCATTCTGACTCTCACTGCCTTTAGCTAGGCCCCTCCCTCATCACAGCTCAGCATAGTCCTGAGC
TCTTATCTATATCCACACACAGTTTCTGACGCTGCCCAGCTATCACCATCCCAAGTCTAAAGAAAAAAAT
AATGGGTTTGCCCATCTCTGTTGATTAGAAAACAAAACAAAATAAAATAAGCCCCTAAGCTCCCAGAAAA
CATGACTAAACCAGCAAGAAGAAGAAAATACAATAGGTATATGAGGAGACTGGTGACACTAAGTGTCTGA
ATGAGGCTTGAGTACAGAAAAGAGGCTCTAGCAGCATAGTGGTTTAGAGGAGATGTTTCTTTCCTTCACA
GATGCCTTAGCCTCAATAAGCTTGCGGTTGTGGAAGTTTACTTGTTTATCACCGGTGACGTCCATGAGCA
AATTAAGAAAAACAACAACAAATGAATGCATATATATGTATATGTATGTGTGTATATATACACATATATA
TATATATTTTTTTTCTTTTCTTACCAGAAGGTTTTAATCCAAATAAGGAGAAGATATGCTTAGAACTGAG
GTAGAGTTTTCATCCATTCTGTCCTGTAAGTATTTTGCATATTCTGGAGACGCAGGAAGAGATCCATCTA
CATATCCCAAAGCTGAATTATGGTAGACAAAACTCTTCCACTTTTAGTGCATCAATTTCTTATTTGTGTA
ATAAGAAAATTGGGAAAACGATCTTCAATATGCTTACCAAGCTGTGATTCCAAATATTACGTAAATACAC
TTGCAAAGGAGGATGTTTTTAGTAGCAATTTGTACTGATGGTATGGGGCCAAGAGATATATCTTAGAGGG
AGGGCTGAGGGTTTGAAGTCCAACTCCTAAGCCAGTGCCAGAAGAGCCAAGGACAGGTACGGCTGTCATC
ACTTAGACCTCACCCTGTGGAGCCACACCCTAGGGTTGGCCAATCTACTCCCAGGAGCAGGGAGGGCAGG
AGCCAGGGCTGGGCATAAAAGTCAGGGCAGAGCCATCTATTGCTTACATTTGCTTCTGACACAACTGTGT
TCACTAGCAACCTCAAACAGACACCATGGGTCATTTCACAGAGGAGGACAAGGCTACTATCACAAGCCTG
TGGGGCAAGGTGAATGTGGAAGATGCTGGAGGAGAAACCCTGGGAAGGTAGGCTCTGGTGACCAGGACAA
GGGAGGGAAGGAAGGACCCTGTGCCTGGCAAAAGTCCAGGTCGCTTCTCAGGATTTGTGGCACCTTCTGA
CTGTCAAACTGTTCTTGTCAATCTCACAGGCTCCTGGTTGTCTACCCATGGACCCAGAGGTTCTTTGACA
GCTTTGGCAACCTGTCCTCTGCCTCTGCCATCATGGGCAACCCCAAAGTCAAGGCACATGGCAAGAAGGT
GCTGACTTCCTTGGGAGATGCCATAAAGCACCTGGATGATCTCAAGGGCACCTTTGCCCAGCTGAGTGAA
CTGCACTGTGACAAGCTGCATGTGGATCCTGAGAACTTCAAGGTGAGTCCAGGAGATGTTTCAGCCCTGT
TGCCTTTAGTCTCGAGGCAACTTAGACAACTGAGTATTGATCTGAGCACAGCAGGGTGTGAGCTGTTTGA
AGATACTGGGGTTGGGGGTGAAGAAACTGCAGAGGACTAACTGGGCTGAGACCCAGTGGTAATGTTTTAG
GGCCTAAGGAGCGCCTCTAAAAATCTAGATGGACAATTTTGACTTTGAGAAAAGAGAGGTGGAAATGAGG
AAAATGACTTTTATTAGATTCCAGTAGAAAGAACTTTCATCTTTCCCTCATTTTTGTTCGTTTTAAAACA
TCTATCTGGAGGCAGGACAAGTATGGTCGTTAAAAAGATGCAGGCAGAAGGCATATATTGGCTCAGTCAA
AGTGGGGAACTTTGGTGGCCAAACATACATTGCTAAGGCTATTCCTATATCAGCTGGACACATATAAAAT
GCTGCTAATGCTTCATTACAAACTTATATCCTTTAATTCCAGATGGGGGCAAAGTATGTCCAGGGGTGAG
GAACAATTGAAACATTTGGGCTGGAGTAGATTTTGAAAGTCAGCTCTGTGTGTGTGTGTGTGTGCGCGCG
CGCGTGTGTGTGTGTGTGTGTCAACGTGTGTTTCTTTTAACGTCTTCAGCCTACAACATACAGGGTTCAT
GGTGGCAAGAAGATAGCAAGATTTAAATTATGGCCAGTGACTAGTGCTTGAAGGGGAACAACTACCTGCA
TTTAATGGGAAGGCAAAATCTCAGGCTTTGAGGGAAGTTAACATAGGCTTGATTCTGGGTAGAAGCTGGG
TGTGTAGTTATCTGGAGGCCAGGCTGGAGCTCTCAGCTCACTATGGGTTCATCTTTATTGTCTCCTTTCA
TCTCAACAGCTCCTGGGAAATGTGCTGGTGACCGTTTTGGCAATCCATTTCGGCAAAGAATTCACCCCTG
AGGTGCAGGCTTCCTGGCAGAAGATGGTGACTGCAGTGGCCAGTGCCCTGTCCTCCAGATACCACTGAGC
CTCTTGCCCATGATTCAGAGCTTTCAAGGATAGGCTTTATTCTGCAAGCAATTCAAATAATAAATCTATT
CTGCTGAGAGATCACACATGATTTTCTTCAGCTCTTTTTTTTACATCTTTTTAAATATATGAGCCACAAA
GGGTTTATATTGAGGGAAGTGTGTATGTGTATTTCTGCATGCCTGTTTGTGTTTGTGGTGTGTGCATGCT
CCTCATTTATTTTTATATGAGATGTGCATTTTGTTGAGCAAATAAAAGCAGTAAAGACACTTGTACACGG
GAGTTCTGCAAGTGGGAGTAAATGGTGTAGGAGAAATCCGGTGGGAAGAAAGACCTCTATAGGACAGGAC
TTCTCAGAAACAGATGTTTTGGAAGAGATGGGAAAAGGTTCAGTGAAGACCTGGGGGCTGGATTGATTGC
AGCTGAGTAGCAAGGATGGTTCTTAATGAAGGGAAAGTGTTCCAAGCTTTAGGAATTCAAGGTTTAGTCA
GGTGTAGCAATTCTATTTTATTAGGAGGAATACTATTTCTAATGGCACTTAGCTTTTCACAGCCCTTGTG
GATGCCTAAGAAAGTGAAATTAATCCCATGCCCTCAAGTGTGCAGATTGGTCACAGCATTTCAAGGGAGA
GACCTCATTGTAAGACTCTGGGGGAGGTGGGGACTTAGGTGTAAGAAATGAATCAGCAGAGGCTCACAAG
TCAGCATGAGCATGTTATGTCTGAGAAACAGACCAGCACTGTGAGATCAAAATGTAGTGGGAAGAATTTG
TACAACATTAATTGGAAGGCTTACTTAATGGAATTTTTGTATAGTTGGATGTTAGTGCATCTCTATAAGT
AAGAGTTTAATATGATGGTGTTACGGACCTAATGTTTGTGTCTCCTCAAAATTCACATGCTGAATCCCCA
ACTCCCAACTGACCTTATCTGTGGGGGAGGCTTTTGAAAAGTAATTAGGTTTAGATGAGCTCATAAGAGC
AGATCCCCATCATAAAATTATTTTCCTTATCAGAAGCAGAGAGACAAGCCATTTCTCTTTCCTCCCGGTG
AGGACACAGTGAGAAGTCCGCCATCTGCAATCCAGGAAGAGAACCCTGACCACGAGTCAGCCTTCAGAAA
TGTGAGAAAAAACTCTGTTGTTGAAGCCACCCAGTCTTTTGTATTTTGTTATAGCACCTTGCACTGAGTA
AGGCAGATGAAGAAGGAGAAAAAAATAAGCTTATCGAAACGCGTCCCCATCCTCACTGACTCCGTCCTGG
AGTTGGATGAGAGATAATGGCCTTACGTTGTGCCAGGGGAGGGTCGGGCTGGATTTAGCAAGATTTACCT
TCTCCAAAGAGCGGTGCTGCAGTGGCACAGCTGCCCACGGAGGTGGGGGGGTCACCGTCCCTGGAGGTGA
TGAAGAACTGTGGGGATGTGGCACTGAGGGACATGGCCAGTGGGCACGGTGGGTGGGTTGGGGTTGGTCT
TGGGGATCTTGGAGGGCTTTTCCAGCCTTCATGATTTGACGATTGTATGAACATCTACATGGCAATTCTC
CAGCTGCCTGTCCCAGTCCTACTGACCCAGCTGTATCTCTCCAGGCAAGCTCTTCCACCCCTTCTGCTTG
CATCCAGACACCATCAAACATGCAGGCTCAGACACAGGGACCAGCAGTGTCTGTGGCCTTTTTGTGCTCC
TCTCCATGCTGGGTTTTAACTTGCTCTTTGTCCTTCTATCCTATCTTCTTATCCTTAAGGCTGTTCTGAA
CGCTGTGACTTGGAGAGTGTCCCAGAGCCCTCAACACCTGCATGTCCCACGTCCATGCTGTCCTGCACTT
CCTTATCCCCAAGATCTGCCTCTCCGTGATGCACTGAATTGGCAAACATGTGTCACCCCAGACCAACAAT
GTCACAGCAAACTCCCCCTTGATAGGACAAGGGGGAATGGCTTTACACTGAGACAGGGGAGGTTTGGGTT
GGATATGAGGAGGCAGTTTTTCCCCCAGAGGGTGGTGACGCACTGAACAGGTTGCCCAAGGAGGCTGTGG
ATGCCCCATCCCTGCAGGCATTCAAGGCCAGGCTGGATGTGGCTCTGGGCAGCCTGGGCTGCTGGTTGAT
GACCCTGCACATAGCAGGGGGTTGGATCTGGATGAGCACTGTGCTCCTTTGCAACCCAGGCCGTTCTATG
ATTCTGTCATTCTAAATCTCTCTTTCAGCCTAAAGCTTTTTCCCCGTATCCCCCCAGGTGTCTGCAGGCT
CAAAGAGCAGCGAGAAGCGTTCAGAGGAAAGCGATCCCGTGCCACCTTCCCCGTGCCCGGGCTGTCCCCG
CACGCTGCCGGCTCGGGGATGCGGGGGGAGCGCCGGACCGGAGCGGAGCCCCGGGCGGCTCGCTGCTGCC
CCCTAGCGGGGGAGGGACGTAATTACATCCCTGGGGGCTTTGGGGGGGGGCTGTCCCTATCGATTACTAG
TTTAGCCATAGAGCCCACCGCATCCCCAGCATGCCTGCTATTGTCTTCCCAATCCTCCCCCTTGCTGTCC
TGCCCCACCCCACCCCCCAGAATAGAATGACACCTACTCAGACAATGCGATGCAATTTCCTCATTTTATT
AGGAAAGGACAGTGGGAGTGGCACCTTCCAGGGTCAAGGAAGGCACGGGGGAGGGGCAAACAACAGATGG
CTGGCAACTAGAAGGCACAGTCGCTCGAAGAGCGGCCGCTCGCTTTACTTGTACAGCTCGTCCATGCCGA
GAGTGATCCCGGCGGCGGTCACGAACTCCAGCAGGACCATGTGATCGCGCTTCTCGTTGGGGTCTTTGCT
CAGGGCGGACTGGGTGCTCAGGTAGTGGTTGTCGGGCAGCAGCACGGGGCCGTCGCCGATGGGGGTGTTC
TGCTGGTAGTGGTCGGCGAGCTGCACGCTGCCGTCCTCGATGTTGTGGCGGATCTTGAAGTTCACCTTGA
TGCCGTTCTTCTGCTTGTCGGCCATGATATAGACGTTGTGGCTGTTGTAGTTGTACTCCAGCTTGTGCCC
CAGGATGTTGCCGTCCTCCTTGAAGTCGATGCCCTTCAGCTCGATGCGGTTCACCAGGGTGTCGCCCTCG
AACTTCACCTCGGCGCGGGTCTTGTAGTTGCCGTCGTCCTTGAAGAAGATGGTGCGCTCCTGGACGTAGC
CTTCGGGCATGGCGGACTTGAAGAAGTCGTGCTGCTTCATGTGGTCGGGGTAGCGGCTGAAGCACTGCAC
GCCGTAGGTCAGGGTGGTCACGAGGGTGGGCCAGGGCACGGGCAGCTTGCCGGTGGTGCAGATGAACTTC
AGGGTCAGCTTGCCGTAGGTGGCATCGCCCTCGCCCTCGCCGGACACGCTGAACTTGTGGCCGTTTACGT
CGCCGTCCAGCTCGACCAGGATGGGCACCACCCCGGTGAACAGCTCCTCGCCCTTGCTCACCATGGGCCC
TGGGTTGGACTCCACGTCTCCCGCCAACTTGAGAAGGTCAAAATTCAAAGTCTGTTTCACCTCGAGGTTT
CGGCCAGCAGGCGGGGAGCCCGAGGTAGCTCCCGCTCCCTTGAGCCAGGCCCCTGCCAGACCTGAGCTCC
CTCCCAAGCCTGGCTTCCCCAACCGGTGGCCTTCATGGGCCAGAAGCCATTCCTTCACGGCTAGCCCTCC
GGAGTAGTTGCCCACGGCTCCGCTGCTGCAGACCACTCTGTGGCACGGGATGAGGATCTTGACAGGATTG
CCTCTCATGGCGCCTCCCACTGCTCGCGCGGCTTTGGGGTTGCCGGCCAGGGCGGCCAATTGCTGGTAAG
AAATCACTTCTCCGAATTTCACAACCTTAAGCAGCTTCCATAACACCTGACGCGTGAACGACTCTTGCTG
GAAAACGGGATGGTGAAGCGCTGGCACGGGGAACTCTTCGATAGCCTCGGGCTGGTGGAAATAGGCATTC
AGCCAGGCTGTGCACTGCATCAGGGGCTCCGGACCTCCGAGAACCGCAGCGGGGGCTGGGACCTCCACGG
CATCAGCTGCAGACGTCCCCTTGCCCAGGAGCTTTATTTCGTGCAGACCCTGCTCACAACCAGACAGCTC
CAGCTTCCCCAAAGGGCTGTCCAGTGTGGTGCGTTTCATTTCACAATCCTTGTCCATGGTGGCCCTAGGC
CCTGGGGAGAGAGGTCGGTGATTCGGTCAACGAGGGAGCCGACTGCCGACGTGCGCTCCGGAGGCTTGCA
GAATGCGGAACACCGCGCGGGCAGGAACAGGGCCCACACTACCGCCCCACACCCCGCCTCCCGCACCGCC
CCTTCCCGGCCGCTGCTCTCGGCGCGCCCCGCTGAGCAGCCGCTATTGGCCACAGCCCATCGCGGTCGGC
GCGCTGCCATTGCTCCCTGGCGCTGTCCGTCTGCGAGGGTACTAGTGAGACGTGCGGCTTCCGTTTGTCA
CGTCCGGCACGCCGCGAACCGCAAGGAACCTTCCCGACTTAGGGGCGGAGCAGGAAGCGTCGCCGGGGGG
CCCACAAGGGTAGCGGCGAAGATCCGGGTGACGCTGCGAACGGACGTGAAGAATGTGCGAGACCCAGGGT
CGGCGCCGCTGCGTTTCCCGGAACCACGCCCAGAGCAGCCGCGTCCCTGCGCAAACCCAGGGCTGCCTTG
GAAAAGGCGCAACCCCAACCCCGTGGAAATAAATCGATAACTAGTGATATCATCATGTCTGGATCCCATC
ACAAAGCTCTGACCTCAATCCTATAGAAAGGAGGAATGAGCCAAAATTCACCCAACTTATTGTGGGAAGC
TTGTGGAAGGCTACTCGAAATGTTTGACCCAAGTTAAACAATTTAAAGGCAATGCTACCAAATACTAATT
GAGTGTATGTTAACTTCTGACCCACTGGGAATGTGATGAAAGAAATAAAAGCTGAAATGAATCATTCTCT
CTACTATTATTCTGATATTTCACATTCTTAAAATAAAGTGGTGATCCTAACTGACCTTAAGACAGGGAAT
CTTTACTCGGATTAAATGTCAGGAATTGTGAAAAAAGTGAGTTTAAATGTATTTGGCTAAGGTGTATGTA
AACTTCCGACTTCAACTGTAGGGGATCCTCTAGGGCCGCCAGTGTGATGGATATCTGCAGAATTCGGCTT
CAGGTACCGTCGACGATGTAGGTCACGGTCTCGAAGCCGCGGTGCGGGTGCCAGGGCGTGCCCTTGGGCT
CCCCGGGCGCGTACTCCACCTCACCCATCTGGTCCATCATGATGAACGGGTCGAGGTGGCGGTAGTTGAT
CCCGGCGAACGCGCGGCGCACCGGGAAGCCCTCGCCCTCGAAACCGCTGGGCGCGGTGGTCACGGTGAGC
ACGGGACGTGCGACGGCGTCGGCGGGTGCGGATACGCGGGGCAGCGTCAGCGGGTTCTCGACGGTCACGG
CGGGCATGTCGACAAGCCGAATTCCAGCACACTGGCGGCCGTTACTAGGTAGCTAGCTCGAGCCTTCGAA
GATCTCCTAGGGAAGTTCCTATACTTTCTAGAGAATAGGAACTTCGGAATAGGAACTTCACCGGTGGGTG
AAAAGCCGAATTCTGCAGATATCAAGCTTATCGATACCGTCGACCTCGAGGGGGGGCCCGGTTAGATCCC
CGGGTACCGAGCTCGAATATCTATGTCGGGTGCGGAGAAAGAGGTAATGAAATGGATTAAGTGGCAGGAT
TAATTAAGTGGCAGGATTAATCTTCGAACGAAGGAGCCATCCAACTAACCGTCATGTTCGGGCAACCGAA
GAAGGGAGTGGCAGGATTTCCTTTGGAGACTTCTGGAATTAGACAGCAGTTTAATGCAAGCATCTAAATT
CTCTTCCTCCCAGAGTCTCATTAAAACTACAGTAAGAGTTTGTGTTTTGTTTTGTTTTTAAAGACAAAAT
CCCACCAGGATAGAGAGAATAGGAGAGGAGATAACAGCATCATAATTTATGAAACTAAAATGCAGATAGA
CCAGGATTAACTGACTACACAGCACCAAGGAAGCTGAATCACAAGACAGCAGAGGAGAAAACTGGAAAGG
ATCGTGGTCTATACGGCAGAATCTTCCCAAGCCTCAGGAGGAGGAGCTCTAGATGCTTATGATGGCAACT
AAAGCCTAAAAGCTAATTCATTTTAAAGTTCTTCCAAATGCATAGGGTTTTATTTTTCCAGACCTGGGTT
CAGATGGGGAATTTGACAAACAATGGAAAGGGGGAAAAACAACAATCTAAACACTGAGTGACAAAGTAAC
AAAGAAATAGTCTAGCTATCAGCCAGTCAAGCCAGCCTTGGCTTTGCTATCCAAAGTAGTCAGTCTAATT
CTACCACCAGTTTCTGTTCCTGTAGCTGTCTACTGCCTGCCAGGGACTCTGCCTTCCCACCCACAACTAC
CAATGGAAGGATGTGGTGACCATACCAGTGGCTGCTGACATCTCCTGCCATGGGAAGCATAATTGCCTCC
AGCAGCCTCCCCCTTAGATCCATCATTTTTGTTGCACTTGGCCTGGGCTGTACTCCCGGCCAATGACTGA
ACATGGTGAGCATAGTAATGCAGGCCCATTTCTGTGAGGAGCAGGACTCCTCCAGTAGGTGACTTTGGCT
CAAGGACTCTCTATTGGCCTGGTTGAACTTTTCCTGAACTGTGCTACTGTCTGAGACTCTTCTTACCCAA
TCCTCTTTCTCGCCCCAATTGTCACAGACCACCTGCATTGTGGTCTGAGTCTCTCCCCACCTTCTCTTGC
TCTTCCCTGTTTATCTTTCACAGGCATTTCCCCCAGTACATTCCTTGAATGTCTAACCCGATACGGGTGC
CTGACTTTTGGCAGACCTAAGCAGACAAAAAGGAGTACTTGGTTACCTAGCTCTTCTTTCTACCACAAAC
ATCGAGGGAACCCTTTTTCCCTCACCCCTCTGCCACACCCCCACTGCCCCAGTGAACAACCACAGAGAGA
GCTGTGGTATAATATTAGGCTGGTGCAAAAGTAATTGCGGTTTTTGCCATTACTTTTAATGGTAAAAACC
GCAATTACTTTTGCACCTACCTAGTATTTGTGTCCCCCCAAATTCATATGTTGAAACCTAACCCACAATA
TGATGTCATTAGGAGGCAAGACCTTGAGGAGGTGATTAGATGATGGGGTGGAGCTCTCCTGAATGAGATT
AGTGCCCTTATAAGAAGAAGCCCAAGGAAGCTACCTTGACTCTTCCATCACATGAGAATGCAGCAAGAAG
GCACCATCTACTAATCAGGAAGAGAGCTCTCACCAGACACTGAATCTGCCAGTGTCTTGATCTTGAAGTT
CCCAGCCTCCAGAACTATGCATAATGCATTTCCATTGTCTCTAAGCCACCCAGCCTATGGTATTTTGTCA
TAGCAGCCTGAACTGACTAAGACAGTGAGCCACATGAGAAGTGCCCCAACCCCTCCCTTAAGCACTTGGC
TCACAGATCAGTGGGTTCATTTCTGCCTGAGTTTTATTGTTATTCTGTAGATTTCTTGGGCTAGATATAT
TTTTCTGTTATTTTCCTTCTTCACCTCAGTCATGAATTGGTTGTTTTAAAAAAGACAATGTAAGTCATGG
GGAAACTCCTGACAACTCTACTCTCCTAGGGTTCCTGATAAAAGGGGATTCAGTTGAGTCCTCTGATGGT
CTCTACCTGCCAAAGTCCAGCAGCCCTTAGCAAACATGCTGCTCGTTTCTGTAGAGAAGGTGCTGGTGTC
CCACCATACTTCTCTCTCCCTCATGAAGGGCTTGCGACCCAGCAAATGGGTGGCTTATATGGGTCTGTTT
CAAAGGAAGAGCCAGCTCTGGGAAGAAAAACGATGAGCATAAGCATAACCTACCACTGTGCCTGGGAAAG
CAGACAACTTTTTTGATGTGTGAATATCTAATGAGAATGGAATCCATCAATTACCTTAAACTTAGGCACA
GTCTTCAAATTCAATATATGTGGGATATACTTTTAGTCAGTTTGTAGACGTTATTTGTAATAAATAATCT
GGCTTCTCTAAAGAAATTATTTTAAGTGTTTGGTTTGGTTTGATTTAATGGTAAAATTATATTTAGTGGC
AGAGAATTATAGCAATGGTGATAAACTATAGAGTGTCATAAGTTCATATCTTATTCTCACATTTGAAGCT
GCCTGCAGATGCATTCAAGATGCAGCCAGAAGTCAGGAGACTCAGGCTGTTATTTGGAGCTCATCATTTT
ACAGCCTTGCTGGACTCCCACTTTCTCAGGGGAAAAATGTGGTGTTGACCCAGATTAGCTCTCCAGGCCC
TGCTGAGTTGGGCACTCTGTAAGCTGGAGGGTCTTCTATTGTCTTCACCTAAGTGTCAATCAACAACCCA
AATGGGCATGGGGGAAGAGGGAGCTGGGCCAATGCCCAGGGTGCCTGGTAGAGAGATACCTTGGGCACTG
GAAGGCACCAGCTTCCCAGAGAGAAGGGGGAGGGCCATGAAAAAGTTGGCTGTAGATGCCAGGGACACTG
GGACTCTCCAGCTGTGTGTTTGTGTCTTCTGAAGACTTATGTTTCATTCCTTTGGAGCATGCATAATCAT
ACACTGTGGGATGTGTTATATAGATTGCTTGATAGTTCACCACTGTAATAAAATACTGTGACTGGAATCT
GCTCCCAGTCTGCCTTTGATAGCACTTGTGCAACACACATTTACTGAGCATTTACAGTGATCCAGGACCT
GTGTTGTGAAAACATTGATGGACAAGGCAGATGGTGGAGCACGTCAGTGAGGATTTTTAACAAAGGCTGG
TAAGTGCTATAAAGGAACATTGTAGGACACTAGAGAACAAAGAACAGGAGAACCTGACTTAGGCTGGGGT
GGGGCGTTGGTTAGAGGAGGCTCCTTGGAGGACATGAGGTTTAAGCTGTGACCTGAGGATGAATAGATGT
TGGCCAGGTGAGGTTAATCTGGGGCTCAAGATCGAGCATTAAGCTTGTCAGCCTTACCAGTAAAAAAGAA
AACCTATTAAAAAAACACCACTCGACACGGCACCAGCTCAATCAGTCACAGTGTAAAAAAGGGCCAAGTG
CAGAGCGAGTATATATAGGACTAAAAAATGACGTAACGGTTAAAGTCCACAAAAAACACCCAGAAAACCG
CACGCGAACCTACGCCCAGAAACGAAAGCCAAAAAACCCACAACTTCCTCAAATCGTCACTTCCGTTTTC
CCACGTTACGTAACTTCCCATTTTAAGAAAACTACAATTCCCAACACATACAAGTTACTCCGCCCTAAAA
CCTACGTCACCCGCCCCGTTCCCACGCCCCGCGCCACGTCACAAACTCCACCCCCTCATTATCATATTGG
CTTCAATCCAAAATAAGGTATATTATTGATGATGTTTAAACTACGGCCCGGTACCCAGCTTTTGTTCCCT
TTAGTGAGGGTTAATTGCGCGCTTGGCGTAATCATGGTCATAGCTGTTTCCTGTGTGAAATTGTTATCCG
CTCACAATTCCACACAACATACGAGCCGGAAGCATAAAGTGTAAAGCCTGGGGTGCCTAATGAGTGAGCT
AACTCACATTAATTGCGTTGCGCTCACTGCCCGCTTTCCAGTCGGGAAACCTGTCGTGCCAGCTGCATTA
ATGAATCGGCCAACGCGCGGGGAGAGGCGGTTTGCGTATTGGGCGCTCTTCCGCTTCCTCGCTCACTGAC
TCGCTGCGCTCGGTCGTTCGGCTGCGGCGAGCGGTATCAGCTCACTCAAAGGCGGTAATACGGTTATCCA
CAGAATCAGGGGATAACGCAGGAAAGAACATGTGAGCAAAAGGCCAGCAAAAGGCCAGGAACCGTAAAAA
GGCCGCGTTGCTGGCGTTTTTCCATAGGCTCCGCCCCCCTGACGAGCATCACAAAAATCGACGCTCAAGT
CAGAGGTGGCGAAACCCGACAGGACTATAAAGATACCAGGCGTTTCCCCCTGGAAGCTCCCTCGTGCGCT
CTCCTGTTCCGACCCTGCCGCTTACCGGATACCTGTCCGCCTTTCTCCCTTCGGGAAGCGTGGCGCTTTC
TCATAGCTCACGCTGTAGGTATCTCAGTTCGGTGTAGGTCGTTCGCTCCAAGCTGGGCTGTGTGCACGAA
CCCCCCGTTCAGCCCGACCGCTGCGCCTTATCCGGTAACTATCGTCTTGAGTCCAACCCGGTAAGACACG
ACTTATCGCCACTGGCAGCAGCCACTGGTAACAGGATTAGCAGAGCGAGGTATGTAGGCGGTGCTACAGA
GTTCTTGAAGTGGTGGCCTAACTACGGCTACACTAGAAGGACAGTATTTGGTATCTGCGCTCTGCTGAAG
CCAGTTACCTTCGGAAAAAGAGTTGGTAGCTCTTGATCCGGCAAACAAACCACCGCTGGTAGCGGTGGTT
TTTTTGTTTGCAAGCAGCAGATTACGCGCAGAAAAAAAGGATCTCAAGAAGATCCTTTGATCTTTTCTAC
GGGGTCTGACGCTCAGTGGAACGAAAACTCACGTTAAGGGATTTTGGTCATGAGATTATCAAAAAGGATC
TTCACCTAGATCCTTTTAAATTAAAAATGAAGTTTTAAATCAATCTAAAGTATATATGAGTAAACTTGGT
CTGACAGTTACCAATGCTTAATCAGTGAGGCACCTATCTCAGCGATCTGTCTATTTCGTTCATCCATAGT
TGCCTGACTCCCCGTCGTGTAGATAACTACGATACGGGAGGGCTTACCATCTGGCCCCAGTGCTGCAATG
ATACCGCGAGACCCACGCTCACCGGCTCCAGATTTATCAGCAATAAACCAGCCAGCCGGAAGGGCCGAGC
GCAGAAGTGGTCCTGCAACTTTATCCGCCTCCATCCAGTCTATTAATTGTTGCCGGGAAGCTAGAGTAAG
TAGTTCGCCAGTTAATAGTTTGCGCAACGTTGTTGCCATTGCTACAGGCATCGTGGTGTCACGCTCGTCG
TTTGGTATGGCTTCATTCAGCTCCGGTTCCCAACGATCAAGGCGAGTTACATGATCCCCCATGTTGTGCA
AAAAAGCGGTTAGCTCCTTCGGTCCTCCGATCGTTGTCAGAAGTAAGTTGGCCGCAGTGTTATCACTCAT
GGTTATGGCAGCACTGCATAATTCTCTTACTGTCATGCCATCCGTAAGATGCTTTTCTGTGACTGGTGAG
TACTCAACCAAGTCATTCTGAGAATAGTGTATGCGGCGACCGAGTTGCTCTTGCCCGGCGTCAATACGGG
ATAATACCGCGCCACATAGCAGAACTTTAAAAGTGCTCATCATTGGAAAACGTTCTTCGGGGCGAAAACT
CTCAAGGATCTTACCGCTGTTGAGATCCAGTTCGATGTAACCCACTCGTGCACCCAACTGATCTTCAGCA
TCTTTTACTTTCACCAGCGTTTCTGGGTGAGCAAAAACAGGAAGGCAAAATGCCGCAAAAAAGGGAATAA
GGGCGACACGGAAATGTTGAATACTCATACTCTTCCTTTTTCAATATTATTGAAGCATTTATCAGGGTTA
TTGTCTCATGAGCGGATACATATTTGAATGTATTTAGAAAAATAAACAAATAGGGGTTCCGCGCACATTT
CCCCGAAAAGTGCCACCTGACGCGCCCTGTAGCGGCGCATTAAGCGCGGCGGGTGTGGTGGTTACGCGCA
GCGTGACCGCTACACTTGCCAGCGCCCTAGCGCCCGCTCCTTTCGCTTTCTTCCCTTCCTTTCTCGCCAC
GTTCGCCGGCTTTCCCCGTCAAGCTCTAAATCGGGGGCTCCCTTTAGGGTTCCGATTTAGTGCTTTACGG
CACCTCGACCCCAAAAAACTTGATTAGGGTGATGGTTCACGTAGTGGGCCATCGCCCTGATAGACGGTTT
TTCGCCCTTTGACGTTGGAGTCCACGTTCTTTAATAGTGGACTCTTGTTCCAAACTGGAACAACACTCAA
CCCTATCTCGGTCTATTCTTTTGATTTATAAGGGATTTTGCCGATTTCGGCCTATTGGTTAAAAAATGAG
CTGATTTAACAAAAATTTAACGCGAATTTTAACAAAATATTAACGCTTACAATTTCCATTCGCCATTCAG
GCTGCGCAACTGTTGGGAAGGGCGATCGGTGCGGGCCTCTTCGCTATTACGCCAGCTGGCGAAAGGGGGA
TGTGCTGCAAGGCGATTAAGTTGGGTAACGCCAGGGTTTTCCCAGTCACGACGTTGTAAAACGACGGCCA
GTGAGCGCGCGTAATACGACTCACTATAGGGCGAATTGGAGCTCCACTACGTAGTTTAAACATCATCAAT
AATATACCTTATTTTGGATTGAAGCCAATATGATAATGAGGGGGTGGAGTTTGTGACGTGGCGCGGGGCG
TGGGAACGGGGCGGGTGACGTAGTAGTGTGGCGGAAGTGTGATGTTGCAAGTGTGGCGGAACACATGTAA
GCGACGGATGTGGCAAAAGTGACGTTTTTGGTGTGCGCCGGTGTACACAGGAAGTGACAATTTTCGCGCG
GTTTTAGGCGGATGTTGTAGTAAATTTGGGCGTAACCGAGTAAGATTTGGCCATTTTCGCGGGAAAACTG
AATAAGAGGAAGTGAAATCTGAATAATTTTGTGTTACTCATAGCGCGTAATATTTGTCTAGGGCCGCGGG
GACTTTGACCGTTTACGTGGAGACTCGCCCAGGTGTTTTTCTCAGGTGTTTTCCGCGTTCCGGGTCAAAG
TTGGCGTTTTGATTC (SEQ ID NO: 178)
The nucleotide sequence of LCR-F8
(Ad5 5′end: 1→440; Sleeping beauty IR:6169→6395, 20868→21095;
HS4-HS1: 6661→10971; Beta promoter: 10991→11653; ET3: 11690→16093;
Mgmt: 18661→19284 (reverse); Ef1a promoter: 19302→20636 (reverse); and
Ad5 3′end: 33317→33514):
CATCATCAATAATATACCTTATTTTGGATTGAAGCCAATATGATAATGAGGGGGTGGAGTTTGTGACGTG
GCGCGGGGCGTGGGAACGGGGCGGGTGACGTAGTAGTGTGGCGGAAGTGTGATGTTGCAAGTGTGGCGGA
ACACATGTAAGCGACGGATGTGGCAAAAGTGACGTTTTTGGTGTGCGCCGGTGTACACAGGAAGTGACAA
TTTTCGCGCGGTTTTAGGCGGATGTTGTAGTAAATTTGGGCGTAACCGAGTAAGATTTGGCCATTTTCGC
GGGAAAACTGAATAAGAGGAAGTGAAATCTGAATAATTTTGTGTTACTCATAGCGCGTAATATTTGTCTA
GGGCCGCGGGGACTTTGACCGTTTACGTGGAGACTCGCCCAGGTGTTTTTCTCAGGTGTTTTCCGCGTTC
CGGGTCAAAGTTGGCGTTTTGATTCGGCCGAAGGATTACATGAGCTTAGAAATGTAATTAGCATAGTGTG
TGGCATAGTGTAGATACCAAATAAATATGATCTCTCCTTCTACTCTTGAAAATGCAAACACATTCTTGGT
GGTCCTAAAATAGCCTGTAACATGGTTTACTCAGCAGCATTTGCTATTCAAGGCAGATCTGCCTTTAGTC
ATTGGCTGCGCTCCTGAACAGCTGTGTGAAAGGCTAACTTTTGTAAACCAAATCAAAATAAAATGCAGCA
AAAATTTGTCACTGAAAGGAAATCCTCAGTATATCCTTTTATGAAATGAAAGATCCCTCATCCAAACTTA
ACTTTTTTAAAAGTGCGCATTTGGAGATATAGCCCTTTCTTATGAATCCTAATTCAATTTTGGCCATAAA
CACACGTTGATGTTCCCCACCCCAAAGCACATAGCAACAAGAGTAGGTTCTATATTGAAAATAATGACAA
TTTAAAAACATGTACTTATTTCACTGTATGTGGACAGTGTCTATGATTGCATCATGAAGTGTCATATAAC
CATGTACGTGTACATGAGAGAGAGATAGAGAGAGAAGTGGTAGGGTGGTGGTGGTAGAGGGGATGGCGAT
AGTAATCATGGTAATGGTAGAGGTGATGGAGGTGGTAATGACGGAGGTAAGGGTGGTAGTGATGATGGTG
GTGGTGGTAATGGTGGTGGATGTGGTGGTGGCAATTGGGATGGTGGGATGGTGGTAGCCATGGTGATGGT
GGTAATGGTGTTGATTTAAAGGGTGGTGGTAGTGAAGGTGAGGGTAGTGGTGGTGGAGGTGGTGGTGCTG
GTAGCAATAGTGATGGTGGTGATGGTGTTGATGAGGGTGTTGGGATCAGGGTGAGTTCCCACAGTATATT
TCATTCTTGTTGTACCACTCTGTCAACAGCACCACTGACTGGGACAGAGGAAGAAGGCACACTCTGAATG
TGTTATTAACAGAAACCTCAAAACAGTCTGTCTCCTTGTAGTCATTCAAAATTATCTTTTTCTTACCTGG
AAAACTGAAACTGAATTACCGGGAAAAACACAGGAGATTTTTGTTTGTTAATATGCTGCCAATAAAGTAA
TTTTATGTCAAATTTAACTACAGGAAAGGGCAAGGCATTTTCTAAGTTCCTTAGATGTCATGTGGCTAAA
AAAAACAAAAGGATGGACAGCAGTTAGATACTGTACACTTAGCTGTTTGAAGCCATATATTCAGAAAGCA
GATGTTGGGAGTTGGTGTTTGAGGACTGATTTCCTGGAGGTATTTTATATAGGCCAAGTTCATTGTTCTA
AACTCTAAGGGCTTGACTTGAGGGAGGAAAAGAGGCAAGAACATGTTTAGTTTTGCTGACAGCATCACAT
GGGCAGCCCTAAGGCTAGACAACTTTAGGGCCTGAAGCTTATTCTAGGAAAGAAGCACCTACAGAGTGGC
ACTGGGCTCCCCTCCACTATAGAGATGAAGTCATATGACAGTAAAGGGCAGGCAGGGCTGCCTAGGGGGC
CCATTGAAATTGCGGCCGCAAATAATGGGCCCGGAGCAAGAGAGAGGGAGGCAATGACAGCAGAGACATG
CCTGCGCCTTGGGTTTGAGTGCCCAGTGGTCAAATCCACTTCCCTGTGGCTGATGCTTGCCTTTCTAACT
TTGGAATTTAGGGGTTGGAGATCTGGTGAGAAGGTAGGAGGGAGATGAGGAGGAGAAGGGAAAGGCAGGA
AGGAAGGGGAGGGAAAGGAAAAGCAAAAGGGGAGGAGGAAGGTTTCCAACAAATTATTCTATATCAACTG
CGGAAATCAAAATTTGTTGCCCAAATCTTAGAAGCTCATGTCCCTCCTCCCCAGAAGTCTGGAATGCAGC
ACTCCAGGGGTAGCTTATAACCCAAATATCTATCTGTAAAAAGAGAAACATTGGGCTTTCGAGCTGTGGA
TTCTCAGTAAAAGCAAGAGGCCTCAGCCTACACAGGCCAGCCCAGAGTTTGAGGAACCCCAGGCCCACAC
CCACAGGGCTGGCCCCTGGGTCTGCATACTCCCTAGAAATGTGCACACTTCTGAGCCTCAACTCTGTCCT
GGAGTCTAACAGCATCCCTCTCCTTCCTGGGGCAGTTCCACCTCCAGAAACCTGTTACCTTGGGCCTTAT
GTCAAGGAAACTGTGGGAAAGAGCTAGGCAGGAATGCAGATGAGGCCAGCATGGGCTCCTAAAAGTTTAG
AAATAGGCAGTGTCATGCTCCCAGGTGCCTGCATAAACCAGCTGAAAAATGGAGCTCCCCTCACCAGCAC
TCTCCCTTCAAACAGACTGTGATTTGCAGGTCACTGGTTTACCAAGCCAGGCTACCCAGGCAGGACCCAG
ATGCCAAGCCCAGTGGTGTCCTGCAAGCTGAGCAGTGCTCAGTTCTTGCAAAAAAAGGTCTGTGTGAAGG
CAAGGCCTCTGCCTGGCTTCTCACCCCAGTTGGGTGTCTGGAACAGGAAGGAGCCCTTACTGCAGAAAAA
GGAGGAGGGAGCAAAGGGAGCGAACAGCTGCGTGCTCCATGGGGAGGATCCCCAAAGTAGAAAGGCGCAT
ACACACTGCAGCCCTTGACCCAGAATGCTCACAGCTACATTACAGATTCAGGTCTCCTCAGTGTAGTGGG
GCTGCTGATGAGACTGTGGCATCCTCAGGGGTCAGGACACACATTTTCCATCACTCTTCTGATGGCAAAA
AACCTCTGAGCCAATGCCAACCTCTGATCATTAAAAAAAAGTGCTCACAGCAGTGTGTGGTTTAGGATCA
TGCCCTGTGTGGTTTGGAACACGTGCACAACCACACCTTGTTCATCACCATCCCAGAAACCCTGACGCAG
GCAAAGAGCAGAGTTATTAACCCTACTTTACTGATGTGGATACTGAGGCCCAGAGGCTCATGCAAGTTAT
CAATAAGTGGCAGGGACAGTTGCCTCTAGATTAACTAGCCCCTAGGATCACCTGGGTCTTGGAAGGGGAC
CCATAAACATGAGCTCCCCTCTCTTGGGGCCAGATTTGCACCTGTGCCGCGCCTTCAGCCTGCATGAAGT
AGGGGCTGCTGGCAAAGACTCAAAGCTGTAAATCTGGGTTTTCTCTTGAGGCTTCTAAGGGAGCTGTTTC
GACAACTCACTCTGTTCCCAGCTGGCTGCCCCTGCATAGGGTTTTAAAGCAGCCTAGCTTTCTGCCAGGC
TTGGCAGTGGACAACGCTGGTCAGAACATCCCAGAGAGCTACCAGAATGAAGTAAGTTTGCTTCTACTCT
TTACCTGTTTATGGGCTGTCTCTGCCACTGGAATGAAAGGCACTGAGAACAGTGCCTGGCCTGCAGAAGG
CCCTGGAAATACCTGAGCTCCTAATCTGGGAATAGGAGTAGGAAGAGCTTTGGAGGCAGGGCACCTGAGT
TTGAGATCTACAACTTCCTGCCTGTGTGACATTGGGAAAGTCTCCATCCTTTCTGAGCCTCAGTCTCCAC
CCTGGGGAAGTGGAAATATCAATCTCTGTGACACAGAAGCAAATGAGCGAATGTGCACAAAGTACCTTGC
ACAAGAGAGACGCTCAAACACTTGCCTCCAGGTTTCACCGAGAACTACAGAGTAAGATAGATTTGTTCCC
AGTGGAGGAAGCCTGGGAATAATTTGCCCCTAGACTATGAATTCCTGGGGCTCAAGATCGAGCACAGGGC
CAGGCACACAGAAGGGACCCTGGAAATGTGGCAGGAGGCCAGAGATAGACAGGCCCTTAGAGCTCATACC
CATGCCCTCTGACCTCAAGAAGAAAGAAACCTGCTCAAAATCTCACAAAGAGCTTGTTCCAACCCTGAAT
CGAGTCTGAGGACTCCTTCCTGAGTCCAGCACTTTTTCTGCAAGAAGTATATGCCTCCAAAGCTGATGGG
CGCAAATCTTGAACCCCGTCACATAAACACAAAGGGAGGAGGTGACTAGAGCTCCTCCTACTGGATATGT
CTAAGGTCACCAGTCTAAAGAAAAGGGATGGATAGAATGAGGCCAGTATTTTTGCAGCCATCCAAATGTC
CACATACGCTGTTACACTGAGGGCTCCTCTCTCCCCCGTCTTCAGCCCTACTTGCATTTAGAGGTGAGAA
AGATATGGGCTGAGGGGTTGTTTTTCATCGTATTGTAGATGGAAAGCACACTGCCCTTGGGGCCATCCAA
ATGTGGACCTTGATGTAGCACCCCACCTTCTGGATGGCCATCCTTCTGAAAGTCACTGAATTTCTCAGAC
TTTATTCTCTTTATCCATAAAGAAGGAGAATAATAATAATCCCCCCACCCTGCCCAACCACTGACTGGTT
GGGAAGCTCAGAAGAAATACTGGGCACGGCATCCCATTGTAATCTATAGAGTGAGTCGCTTCTTAATATT
AAATGGCTGAACACAGAAGATGTGCAAAAAGTACTGTGTCCCCTTCCTCCTCCAACTGAACATTTCATGC
CCTTTGCACCCTCATTTTGTCTAGGAGCTGCCTTATGAAGGGAATAGGTACCTGCTCCGAGCTGGAGGAA
TCTTTGCCACTTATGGTGGGGTATGGACTGAGACAGAGATGGCATGTGACATGCGCACTGAGTCTCAACT
CCATGCAGGCTCTGGAGCACTCTCAAATTGGAGTACTAATGCCTTTTAAATTCTCACACTAGCAATCCTT
TGACCTACTGATCTAGGGATCTAGGGAAAGAATCGTGATCTTAACTTCAAAGGGAAGGACAAAATGTTCT
GCCTCCTGTTAAAACTCCATACACTAAGTGCAGAGACTGGATGCCTTATTAACCTTGGGTAGATGCCCAA
ATGTTCAAAAGGTCAAACTCTTCTGTTCCCCAGATCGCCAGAGTCATTAACCAGTCACACTATTAAATGA
ATGAACAGATGCTGAAAAGGTACTTGCATTACTGAGATTTCTTATGGTGATGGCCCCTGCCTGATATGTA
TTCAGCATTTTGTAGTTTTCAATGTGCATTAGAGTATAGTGGTGATGACATTGGCCTCTGAGTTTGCCAC
TTCTTATATCTGTGACTTTGGTCAAATTGCTTAATCTCTCTGAGTCTCGGTTTCCTGGAGATAATAATAG
CTTCTTCTTCCCAGGGTTATCATGAGGATTACAGGAGATAATGCCCCAAAAATGCTTAGTAAAGTGCCTA
GCACCTAGTCAATGCTGAATTAAAGGTGGTTATTCTTACTTTTCGTTCATTTGAACTTTGTTCTCAGGGA
GGGCAAAGGATAGACAAAGCCCCATAGCTAGTGAGGAGTAGCTGCAAGACTAGAACCCAGGTGTTCTGAG
CCCTAGTCTTAGGCCAAGAACAACTGTTACGTGAGATGCACGTTTTCCTTCAAGGGAGCTCACAATTATT
TCCATGTAAATTCAAGGACTGCTAAAAGAGAACTCTCCTCTGGGACTGATAAACATCTAGTCGAGTATCG
ACAAATAGGGGTTCCGCGCACATTTCCCCGAAAAGTGCCACCTGACGTAACTATAACGGTCCTAAGGTAG
CGAAAGCTTGCATGCCTGCAGGTCGACTCTAGAGGATCAAACCTAGCCACCGCGGTGGCGGCCGGCTAGC
CGGCTAGCCGGCTAGCCCTAGAACTAGTAACGGCCGCCAGTGTGCTGGAATTCGGCTTGTAAGGTACCGG
TGAAGTTCCTATACTTTCTAGAGAATAGGAACTTCGGAATAGGAACTTCTACCTAGATGCATGCTCGAGC
GGCCCCTACAGTTGAAGTCGGAAGTTTACATACACTTAAGTTGGAGTCATTAAAACTCGTTTTTCAACTA
CTCCACAAATTTCTTGTTAACAAACAATAGTTTTGGCAAGTCAGTTAGGACATCTACTTTGTGCATGACA
CAAGTCATTTTTCCAACAATTGTTTACAGACAGATTATTTCACTTATAATTCACTGTATCACAATTCCAG
TGGGTCAGAAGTTTACATACACTAAGTTGACTGTGCCTTTAAACAGCTTGGAAAATTCCAGAAAATGATG
TCATGGCTTTAGAAGCTTCTGATAGACTAATTGACATCATTTGAGTCAATTGGAGGTGTACCTGTGGATG
TATTTCAAGGAATTCTGTGGAATGTGTGTCAGTTAGGGTGTGGAAAGTCCCCAGGCTCCCCAGCAGGCAG
AAGTATGCAAAGCATGCATATCGATACTAGTTTATAAGATCTCGAGCTAGCGGCCGTTTGTTAATTAAGT
CGACGGTACCGTCAAGGCTGCAGTGAGACATGATCTTGCCACTGCACTCCAGCCTGGACAGCAGAGTGAA
ACCTTGCCTCACGAAACAGAATACAAAAACAAACAAACAAAAAACTGCTCCGCAATGCGCTTCCTTGATG
CTCTACCACATAGGTCTGGGTACTTTGTACACATTATCTCATTGCTGTTCATAATTGTTAGATTAATTTT
GTAATATTGATATTATTCCTAGAAAGCTGAGGCCTCAAGATGATAACTTTTATTTTCTGGACTTGTAATA
GCTTTCTCTTGTATTCACCATGTTGTAACTTTCTTAGAGTAGTAACAATATAAAGTTATTGTGAGTTTTT
GCAAACACAGCAAACACAACGACCCATATAGACATTGATGTGAAATTGTCTATTGTCAATTTATGGGAAA
ACAAGTATGTACTTTTTCTACTAAGCCATTGAAACAGGAATAACAGAACAAGATTGAAAGAATACATTTT
CCGAAATTACTTGAGTATTATACAAAGACAAGCACGTGGACCTGGGAGGAGGGTTATTGTCCATGACTGG
TGTGTGGAGACAAATGCAGGTTTATAATAGATGGGATGGCATCTAGCGCAATGACTTTGCCATCACTTTT
AGAGAGCTCTTGGGGACCCCAGTACACAAGAGGGGACGCAGGGTATATGTAGACATCTCATTCTTTTTCT
TAGTGTGAGAATAAGAATAGCCATGACCTGAGTTTATAGACAATGAGCCCTTTTCTCTCTCCCACTCAGC
AGCTATGAGATGGCTTGCCCTGCCTCTCTACTAGGCTGACTCACTCCAAGGCCCAGCAATGGGCAGGGCT
CTGTCAGGGCTTTGATAGCACTATCTGCAGAGCCAGGGCCGAGAAGGGGTGGACTCCAGAGACTCTCCCT
CCCATTCCCGAGCAGGGTTTGCTTATTTATGCATTTAAATGATATATTTATTTTAAAAGAAATAACAGGA
GACTGCCCAGCCCTGGCTGTGACATGGAAACTATGTAGAATATTTTGGGTTCCATTTTTTTTTCCTTCTT
TCAGTTAGAGGAAAAGGGGCTCAGGATCCACTTGCCCAGTGTTCTTCCTTAGTTCCTACCTTCGACCTTG
ATCCTCCTTTATCTTCCTGAACCCTGCTGAGATGATCTATGTGGGGAGAATGGCTTCTTTGAGAAACATC
TTCTTCGTTAGTGGCCTGCCCCTCATTCCCACTTTAATATCCAGAATCACTATAAGAAGAATATAATAAG
AGGAATAACTCTTATTATAGGTAAGGGAAAATTAAGAGGCATACGTGATGGGATGAGTAAGAGAGGAGAG
GGAAGGATTAATGGACGATAAAATCTACTACTATTTGTTGAGACCTTTTATAGTCTAATCAATTTTGCTA
TTGTTTTCCATCCTCACGCTAACTCCATAAAAAAACACTATTATTATCTTTATTTTGCCATGACAAGACT
GAGCTCAGAAGAGTCAAGCATTTGCCTAAGGTCGGACATGTCAGAGGCAGTGCCAGACCTATGTGAGACT
CTGCAGCTACTGCTCATGGGCCCTGTGCTGCACTGATGAGGAGGATCAGATGGATGGGGCAATGAAGCAA
AGGAATCATTCTGTGGATAAAGGAGACAGCCATGAAGAAGTCTATGACTGTAAATTTGGGAGCAGGAGTC
TCTAAGGACTTGGATTTCAAGGAATTTTGACTCAGCAAACACAAGACCCTCACGGTGACTTTGCGAGCTG
GTGTGCCAGATGTGTCTATCAGAGGTTCCAGGGAGGGTGGGGTGGGGTCAGGGCTGGCCACCAGCTATCA
GGGCCCAGATGGGTTATAGGCTGGCAGGCTCAGATAGGTGGTTAGGTCAGGTTGGTGGTGCTGGGTGGAG
TCCATGACTCCCAGGAGCCAGGAGAGATAGACCATGAGTAGAGGGCAGACATGGGAAAGGTGGGGGAGGC
ACAGCATAGCAGCATTTTTCATTCTACTACTACATGGGACTGCTCCCCTATACCCCCAGCTAGGGGCAAG
TGCCTTGACTCCTATGTTTTCAGGATCATCATCTATAAAGTAAGAGTAATAATTGTGTCTATCTCATAGG
GTTATTATGAGGATCAAAGGAGATGCACACTCTCTGGACCAGTGGCCTAACAGTTCAGGACAGAGCTATG
GGCTTCCTATGTATGGGTCAGTGGTCTCAATGTAGCAGGCAAGTTCCAGAAGATAGCATCAACCACTGTT
AGAGATATACTGCCAGTCTCAGAGCCTGATGTTAATTTAGCAATGGGCTGGGACCCTCCTCCAGTAGAAC
CTTCTAACCAGCTGCTGCAGTCAAAGTCGAATGCAGCTGGTTAGACTTTTTTTAATGAAAGCTTGCATGC
AGCACTTTGGGAGGCTGAGGTGGGTGGACTGCTTGGAGCTCAGGAGTTCAAGACCATCTTGGACAACATG
GTGATACCCTGCCTCTACAAAAAGTACAAAAATTAGCCTGGCATGGTGGTGTGCACCTGTAATCCCAGCT
ATTAGGGTGGCTGAGGCAGGAGAATTGCTTGAACCCGGGAGGCGGAGGTTGCAGTGAGCTGAGATCGTGC
CACTGCACTCCAGCCTGGGGGACAGAGCACATTATAATTAACTGTTATTTTTTACTTGGACTCTTGTGGG
GAATAAGATACATGTTTTATTCTTATTTATGATTCAAGCACTGAAAATAGTGTTTAGCATCCAGCAGGTG
CTTCAAAACCATTTGCTGAATGATTACTATACTTTTTACAAGCTCAGCTCCCTCTATCCCTTCCAGCATC
CTCATCTCTGATTAAATAAGCTTCAGTTTTTCCTTAGTTCCTGTTACATTTCTGTGTGTCTCCATTAGTG
ACCTCCCATAGTCCAAGCATGAGCAGTTCTGGCCAGGCCCCTGTCGGGGTCAGTGCCCCACCCCCGCCTT
CTGGTTCTGTGTAACCTTCTAAGCAAACCTTCTGGCTCAAGCACAGCAATGCTGAGTCATGATGAGTCAT
GCTGAGGCTTAGGGTGTGTGCCCAGATGTTCTCAGCCTAGAGTGATGACTCCTATCTGGGTCCCCAGCAG
GATGCTTACAGGGCAGATGGCAAAAAAAAGGAGAAGCTGACCACCTGACTAAAACTCCACCTCAAACGGC
ATCATAAAGAAAATGGATGCCTGAGACAGAATGTGACATATTCTAGAATATATTATTTCCTGAATATATA
TATATATATACACATATACCATATGAAACACCTCTAGGCTATAAGGCAACAGAGCTCCTTTTTTTTTTTT
CTGTGCTTTCCTGGCTGTCCAAATCTCTAATGATAAGCATACTTCTATTCAATGAGAATATTCTGTAAGA
TTATAGTTAAGAATTGTGGGAGCCATTCCGTCTCTTATAGTTAAATTTGAGCTTCTTTTATGATCACTGT
TTTTTTAATATGCTTTAAGTTCTGGGGTACATGTGCCATGGTGGTTTGCTGCACCCATCAACCCGTCATC
TACATTAGGTATTTCTCCTAATGCTATCCTTCCCCTAGCCCCCCACCCCCAACAGGCCCCAGTGTGTGAT
GTTCCCCTCCCTGTGTCCATGGATCACTGGTTTTTTTTTGTTTTTTTTTTTTTTTTAAAGTCTCAGTTAA
ATTTTTGGAATGTAATTTATTTTCCTGGTATCCTAAGGACTTGCAAGTTATCTGGTCACTTTAGCCCTCA
CGTTTTGATGATAATCACATATTTGTAAACACAACACACACACACACACACACACACATATATATATATA
TAAAACATATATATACATAAACACACATAACATATTTATCGGGCATTTCTGAGCAACTAATCATGCAGGA
CTCTCAAACACTAACCTATAGCCTTTTCTATGTATCTACTTGTGTAGAAACCAAGCGTGGGGACTGAGAA
GGCAATAGCAGGAGCATTCTGACTCTCACTGCCTTTAGCTAGGCCCCTCCCTCATCACAGCTCAGCATAG
TCCTGAGCTCTTATCTATATCCACACACAGTTTCTGACGCTGCCCAGCTATCACCATCCCAAGTCTAAAG
AAAAAAATAATGGGTTTGCCCATCTCTGTTGATTAGAAAACAAAACAAAATAAAATAAGCCCCTAAGCTC
CCAGAAAACATGACTAAACCAGCAAGAAGAAGAAAATACAATAGGTATATGAGGAGACTGGTGACACTAA
GTGTCTGAATGAGGCTTGAGTACAGAAAAGAGGCTCTAGCAGCATAGTGGTTTAGAGGAGATGTTTCTTT
CCTTCACAGATGCCTTAGCCTCAATAAGCTTGCGGTTGTGGAAGTTTACTTGTTTATCACCGGTGACGTC
CATGAGCAAATTAAGAAAAACAACAACAAATGAATGCATATATATGTATATGTATGTGTGTATATATACA
CATATATATATATATTTTTTTTCTTTTCTTACCAGAAGGTTTTAATCCAAATAAGGAGAAGATATGCTTA
GAACTGAGGTAGAGTTTTCATCCATTCTGTCCTGTAAGTATTTTGCATATTCTGGAGACGCAGGAAGAGA
TCCATCTACATATCCCAAAGCTGAATTATGGTAGACAAAACTCTTCCACTTTTAGTGCATCAATTTCTTA
TTTGTGTAATAAGAAAATTGGGAAAACGATCTTCAATATGCTTACCAAGCTGTGATTCCAAATATTACGT
AAATACACTTGCAAAGGAGGATGTTTTTAGTAGCAATTTGTACTGATGGTATGGGGCCAAGAGATATATC
TTAGAGGGAGGGCTGAGGGTTTGAAGTCCAACTCCTAAGCCAGTGCCAGAAGAGCCAAGGACAGGTACGG
CTGTCATCACTTAGACCTCACCCTGTGGAGCCACACCCTAGGGTTGGCCAATCTACTCCCAGGAGCAGGG
AGGGCAGGAGCCAGGGCTGGGCATAAAAGTCAGGGCAGAGCCATCTATTGCTTACATTTGCTTCTGACAC
AACTGTGTTCACTAGCAACCTCAAACAGACACCTCGAGTTTCGATTGCTAGCTTCGAACTCGAGCCACCA
TGCAGCTAGAGCTCTCCACCTGTGTCTTTCTGTGTCTCTTGCCACTCGGCTTTAGTGCCATCAGGAGATA
CTACCTGGGCGCAGTGGAACTGTCCTGGGACTACCGGCAAAGTGAACTCCTCCGTGAGCTGCACGTGGAC
ACCAGATTTCCTGCTACAGCGCCAGGAGCTCTTCCGTTGGGCCCGTCAGTCCTGTACAAAAAGACTGTGT
TCGTAGAGTTCACGGATCAACTTTTCAGCGTTGCCAGGCCCAGGCCACCATGGATGGGTCTGCTGGGTCC
TACCATCCAGGCTGAGGTTTACGACACGGTGGTCGTTACCCTGAAGAACATGGCTTCTCATCCCGTTAGT
CTTCACGCTGTCGGCGTCTCCTTCTGGAAATCTTCCGAAGGCGCTGAATATGAGGATCACACCAGCCAAA
GGGAGAAGGAAGACGATAAAGTCCTTCCCGGTAAAAGCCAAACCTACGTCTGGCAGGTCCTGAAAGAAAA
TGGTCCAACAGCCTCTGACCCACCATGTCTTACCTACTCATACCTGTCTCACGTGGACCTGGTGAAAGAC
CTGAATTCGGGCCTCATTGGAGCCCTGCTGGTTTGTAGAGAAGGGAGTCTGACCAGAGAAAGGACCCAGA
ACCTGCACGAATTTGTACTACTTTTTGCTGTCTTTGATGAAGGGAAAAGTTGGCACTCAGCAAGAAATGA
CTCCTGGACACGGGCCATGGATCCCGCACCTGCCAGGGCCCAGCCTGCAATGCACACAGTCAATGGCTAT
GTCAACAGGTCTCTGCCAGGTCTGATCGGATGTCATAAGAAATCAGTCTACTGGCACGTGATTGGAATGG
GCACCAGCCCGGAAGTGCACTCCATTTTTCTTGAAGGCCACACGTTTCTCGTGAGGCACCATCGCCAGGC
TTCCTTGGAGATCTCGCCACTAACTTTCCTCACTGCTCAGACATTCCTGATGGACCTTGGCCAGTTCCTA
CTGTTTTGTCATATCTCTTCCCACCACCATGGTGGCATGGAGGCTCACGTCAGAGTAGAAAGCTGCGCCG
AGGAGCCCCAGCTGCGGAGGAAAGCTGATGAAGAGGAAGATTATGATGACAATTTGTACGACTCGGACAT
GGACGTGGTCCGGCTCGATGGTGACGACGTGTCTCCCTTTATCCAAATCCGCTCAGTTGCCAAGAAGCAT
CCTAAAACTTGGGTACATTACATTGCTGCTGAAGAGGAGGACTGGGACTATGCTCCCTTAGTCCTCGCCC
CCGATGACAGAAGTTATAAAAGTCAATATTTGAACAATGGCCCTCAGCGGATTGGTAGGAAGTACAAAAA
AGTCCGATTTATGGCATACACAGATGAAACCTTTAAGACGCGTGAAGCTATTCAGCATGAATCAGGAATC
TTGGGACCTTTACTTTATGGGGAAGTTGGAGACACACTGTTGATTATATTTAAGAATCAAGCAAGCAGAC
CATATAACATCTACCCTCACGGAATCACTGATGTCCGTCCTTTGTATTCAAGGAGATTACCAAAAGGTGT
AAAACATTTGAAGGATTTTCCAATTCTGCCAGGAGAAATATTCAAATATAAATGGACAGTGACTGTAGAA
GATGGGCCAACTAAATCAGATCCGCGGTGCCTGACCCGCTATTACTCTAGTTTCGTTAATATGGAGAGAG
ATCTAGCTTCAGGACTCATTGGCCCTCTCCTCATCTGCTACAAAGAATCTGTAGATCAAAGAGGAAACCA
GATAATGTCAGACAAGAGGAATGTCATCCTGTTTTCTGTATTTGATGAGAACCGAAGCTGGTACCTCACA
GAGAATATACAACGCTTTCTCCCCAATCCAGCTGGAGTGCAGCTTGAGGATCCAGAGTTCCAAGCCTCCA
ACATCATGCACAGCATCAATGGCTATGTTTTTGATAGTTTGCAGTTGTCAGTTTGTTTGCATGAGGTGGC
ATACTGGTACATTCTAAGCATTGGAGCACAGACTGACTTCCTTTCTGTCTTCTTCTCTGGATATACCTTC
AAACACAAAATGGTCTATGAAGACACACTCACCCTATTCCCATTCTCAGGAGAAACTGTCTTCATGTCGA
TGGAAAACCCAGGTCTATGGATTCTGGGGTGCCACAACTCAGACTTTCGGAACAGAGGCATGACCGCCTT
ACTGAAGGTTTCTAGTTGTGACAAGAACACTGGTGATTATTACGAGGACAGTTATGAAGATATTTCAGCA
TACTTGCTGAGTAAAAACAATGCCATTGAACCTAGGAGCTTTGCCCAGAATTCAAGACCCCCTAGTGCGA
GCGCTCCAAAGCCTCCGGTCCTGCGACGGCATCAGAGGGACATAAGCCTTCCTACTTTTCAGCCGGAGGA
AGACAAAATGGACTATGATGATATCTTCTCAACTGAAACGAAGGGAGAAGATTTTGACATTTACGGTGAG
GATGAAAATCAGGACCCTCGCAGCTTTCAGAAGAGAACCCGACACTATTTCATTGCTGCGGTGGAGCAGC
TCTGGGATTACGGGATGAGCGAATCCCCCCGGGCGCTAAGAAACAGGGCTCAGAACGGAGAGGTGCCTCG
GTTCAAGAAGGTGGTCTTCCGGGAATTTGCTGACGGCTCCTTCACGCAGCCGTCGTACCGCGGGGAACTC
AACAAACACTTGGGGCTCTTGGGACCCTACATCAGAGCGGAAGTTGAAGACAACATCATGGTAACTTTCA
AAAACCAGGCGTCTCGTCCCTATTCCTTCTACTCGAGCCTTATTTCTTATCCGGATGATCAGGAGCAAGG
GGCAGAACCTCGACACAACTTCGTCCAGCCAAATGAAACCAGAACTTACTTTTGGAAAGTGCAGCATCAC
ATGGCACCCACAGAAGACGAGTTTGACTGCAAAGCCTGGGCCTACTTTTCTGATGTTGACCTGGAAAAAG
ATGTGCACTCAGGCTTGATCGGCCCCCTTCTGATCTGCCGCGCCAACACCCTGAACGCTGCTCACGGTAG
ACAAGTGACCGTGCAAGAATTTGCTCTGTTTTTCACTATTTTTGATGAGACAAAGAGCTGGTACTTCACT
GAAAATGTGGAAAGGAACTGCCGGGCCCCCTGCCATCTGCAGATGGAGGACCCCACTCTGAAAGAAAACT
ATCGCTTCCATGCAATCAATGGCTATGTGATGGATACACTCCCTGGCTTAGTAATGGCTCAGAATCAAAG
GATCCGATGGTATCTGCTCAGCATGGGCAGCAATGAAAATATCCATTCGATTCATTTTAGCGGACACGTG
TTCAGTGTACGGAAAAAGGAGGAGTATAAAATGGCCGTGTACAATCTCTATCCGGGTGTCTTTGAGACAG
TGGAAATGCTACCGTCCAAAGTTGGAATTTGGCGAATAGAATGCCTGATTGGCGAGCACCTGCAAGCTGG
GATGAGCACGACTTTCCTGGTGTACAGCAAGAAGTGTCAGACTCCCCTGGGAATGGCTTCTGGACACATT
AGAGATTTTCAGATTACAGCTTCAGGACAATATGGACAGTGGGCCCCAAAGCTGGCCAGACTTCATTATT
CCGGATCAATCAATGCCTGGAGCACCAAGGAGCCCTTTTCTTGGATCAAGGTGGATCTGTTGGCACCAAT
GATTATTCACGGCATCAAGACCCAGGGTGCCCGTCAGAAGTTCTCCAGCCTCTACATCTCTCAGTTTATC
ATCATGTATAGTCTTGATGGGAAGAAGTGGCAGACTTATCGAGGAAATTCCACTGGAACCTTAATGGTCT
TCTTTGGCAATGTGGATTCATCTGGGATAAAACACAATATTTTTAACCCTCCAATTATTGCTCGATACAT
CCGTTTGCACCCAACTCATTATAGCATTCGCAGCACTCTTCGCATGGAGTTGATGGGCTGTGATTTAAAT
AGTTGCAGCATGCCATTGGGAATGGAGAGTAAAGCAATATCAGATGCACAGATTACTGCTTCATCCTACT
TTACCAATATGTTTGCCACCTGGTCTCCTTCAAAAGCTCGACTTCACCTCCAAGGGAGGAGTAATGCCTG
GAGACCTCAGGTGAATAATCCAAAAGAGTGGCTGCAAGTGGACTTCCAGAAGACAATGAAAGTCACAGGA
GTAACTACTCAGGGAGTAAAATCTCTGCTTACCAGCATGTATGTGAAGGAGTTCCTCATCTCCAGCAGTC
AAGATGGCCATCAGTGGACTCTCTTTTTTCAGAATGGCAAAGTAAAGGTTTTTCAGGGAAATCAAGACTC
CTTCACACCTGTGGTGAACTCTCTAGACCCACCGTTACTGACTCGCTACCTTCGAATTCACCCCCAGAGT
TGGGTGCACCAGATTGCCCTGAGGATGGAGGTTCTGGGCTGCGAGGCACAGGACCTCTACTGAGGGCGGC
CGCCACCCCACCAGTGCAGGCTGCCTATCAGAAAGTGGTGGCTGGTGTGGCTAATGCCCTGGCCCACAAG
TATCACTAAGCTCGCTTTCTTGCTGTCCAATTTCTATTAAAGGTTCCTTTGTTCCCTAAGTCCAACTACT
AAACTGGGGGATATTATGAAGGGCCTTGAGCATCTGGATTCTGCCTAATAAAAAACATTTATTTTCATTG
CAATGATGTATTTAAATTATTTCTGAATATTTTACTAAAAAGGGAATGTGGGAGGTCAGTGCATTTAAAA
CATAAAGAAATGAAGAGCTAGTTCAAACCTTGGGAAAATACACTATATCTTAAACTCCATGAAAGAAGGT
GAGGCTGCAAACAGCTAATGCACATTGGCAACAGCCCCTGATGCCTATGCCTTATTCATCCCTCAGAAAA
GGATTCAAGTAGAGGCTTGATTTGGAGGTTAAAGTTTTGCTATGCTGTATTTTACATTACTTATTGTTTT
AGCTGTCCTCATGAATGTCTTTTCACTACCCATTTGCTTATCCTGCATCTCTCAGCCTTGACTCCACTCA
GTTCTCTTGCTTAGAGATACCACCTTTCCCCTGAAGTGTTCCTTCCATGTTTTACGGCGAGATGGTTTCT
CCTCGCCTGGCCACTCAGCCTTAGTTGTCTCTGTTGTCTTATAGAGGTCTACTTGAAGAAGGAAAAACAG
GGGGCATGGTTTGACTGTCCTGTGAGCCCTTCTTCCCTGCCTCCCCCACTCACAGTGACCCGGAATCTGC
AGTGCTAGTCTCCCGGAACTATCACTCTTTCACAGTCTGCTTTGGAAGGACTGGGCTTAGTATGAAAAGT
TAGGACTGAGAAGAATTTGAAAGGGGGCTTTTTGTAGCTTGATATTCACTACTGTCTTATTACCCTATCA
TAGGCCCACCCCAAATGGAAGTCCCATTCTTCCTCAGGATGTTTAAGATTAGCATTCAGGAAGAGATCAG
AGGTCTGCTGGCTCCCTTATCATGTCCCTTATGGTGCTTCTGGCTCTGCAGTTATTAGCATAGTGTTACC
ATCAACCACCTTAACTTCATTTTTCTTATTCAATACCTAGAAAGCTTATCGACCCCATCCTCACTGACTC
CGTCCTGGAGTTGGATGAGAGATAATGGCCTTACGTTGTGCCAGGGGAGGGTCGGGCTGGATTTAGCAAG
ATTTACCTTCTCCAAAGAGCGGTGCTGCAGTGGCACAGCTGCCCACGGAGGTGGGGGGGTCACCGTCCCT
GGAGGTGATGAAGAACTGTGGGGATGTGGCACTGAGGGACATGGCCAGTGGGCACGGTGGGTGGGTTGGG
GTTGGTCTTGGGGATCTTGGAGGGCTTTTCCAGCCTTCATGATTTGACGATTGTATGAACATCTACATGG
CAATTCTCCAGCTGCCTGTCCCAGTCCTACTGACCCAGCTGTATCTCTCCAGGCAAGCTCTTCCACCCCT
TCTGCTTGCATCCAGACACCATCAAACATGCAGGCTCAGACACAGGGACCAGCAGTGTCTGTGGCCTTTT
TGTGCTCCTCTCCATGCTGGGTTTTAACTTGCTCTTTGTCCTTCTATCCTATCTTCTTATCCTTAAGGCT
GTTCTGAACGCTGTGACTTGGAGAGTGTCCCAGAGCCCTCAACACCTGCATGTCCCACGTCCATGCTGTC
CTGCACTTCCTTATCCCCAAGATCTGCCTCTCCGTGATGCACTGAATTGGCAAACATGTGTCACCCCAGA
CCAACAATGTCACAGCAAACTCCCCCTTGATAGGACAAGGGGGAATGGCTTTACACTGAGACAGGGGAGG
TTTGGGTTGGATATGAGGAGGCAGTTTTTCCCCCAGAGGGTGGTGACGCACTGAACAGGTTGCCCAAGGA
GGCTGTGGATGCCCCATCCCTGCAGGCATTCAAGGCCAGGCTGGATGTGGCTCTGGGCAGCCTGGGCTGC
TGGTTGATGACCCTGCACATAGCAGGGGGTTGGATCTGGATGAGCACTGTGCTCCTTTGCAACCCAGGCC
GTTCTATGATTCTGTCATTCTAAATCTCTCTTTCAGCCTAAAGCTTTTTCCCCGTATCCCCCCAGGTGTC
TGCAGGCTCAAAGAGCAGCGAGAAGCGTTCAGAGGAAAGCGATCCCGTGCCACCTTCCCCGTGCCCGGGC
TGTCCCCGCACGCTGCCGGCTCGGGGATGCGGGGGGAGCGCCGGACCGGAGCGGAGCCCCGGGCGGCTCG
CTGCTGCCCCCTAGCGGGGGAGGGACGTAATTACATCCCTGGGGGCTTTGGGGGGGGGCTGTCCCTATCG
ATTACTAGTTTTACCACATTTGTAGAGGTTTTACTTGCTTTAAAAAACCTCCCACACCTCCCCCTGAACC
TGAAACATAAAATGAATGCAATTGTTGTTGTTAACTTGTTTATTGCAGCTTATAATGGTTACAAATAAAG
CAATAGCATCACAAATTTCACAAATAAAGCATTTTTTTCACTGCATTCTAGTTGTGGTTTGTCCAAACTC
ATCAATGTATCTTATCATGTCTGCTCGAAGAGCGGCCGCTTCAGTTTCGGCCAGCAGGCGGGGAGCCCGA
GGTAGCTCCCGCTCCCTTGAGCCAGGCCCCTGCCAGACCTGAGCTCCCTCCCAAGCCTGGCTTCCCCAAC
CGGTGGCCTTCATGGGCCAGAAGCCATTCCTTCACGGCTAGCCCTCCGGAGTAGTTGCCCACGGCTCCGC
TGCTGCAGACCACTCTGTGGCACGGGATGAGGATCTTGACAGGATTGCCTCTCATGGCGCCTCCCACTGC
TCGCGCGGCTTTGGGGTTGCCGGCCAGGGCGGCCAATTGCTGGTAAGAAATCACTTCTCCGAATTTCACA
ACCTTAAGCAGCTTCCATAACACCTGACGCGTGAACGACTCTTGCTGGAAAACGGGATGGTGAAGCGCTG
GCACGGGGAACTCTTCGATAGCCTCGGGCTGGTGGAAATAGGCATTCAGCCAGGCTGTGCACTGCATCAG
GGGCTCCGGACCTCCGAGAACCGCAGCGGGGGCTGGGACCTCCACGGCATCAGCTGCAGACGTCCCCTTG
CCCAGGAGCTTTATTTCGTGCAGACCCTGCTCACAACCAGACAGCTCCAGCTTCCCCAAAGGGCTGTCCA
GTGTGGTGCGTTTCATTTCACAATCCTTGTCCATGGTGGCGACCGTCTAGCTCACGACACCTGAAATGGA
AGAAAAAAACTTTGAACCACTGTCTGAGGCTTGAGAATGAACCAAGATCCAAACTCAAAAAGGGCAAATT
CCAAGGAGAATTACATCAAGTGCCAAGCTGGCCTAACTTCAGTCTCCACCCACTCAGTGTGGGGAAACTC
CATCGCATAAAACCCCTCCCCCCAACCTAAAGACGACGTACTCCAAAAGCTCGAGAACTAATCGAGGTGC
CTGGACGGCGCCCGGTACTCCGTGGAGTCACATGAAGCGACGGCTGAGGACGGAAAGGCCCTTTTCCTTT
GTGTGGGTGACTCACCCGCCCGCTCTCCCGAGCGCCGCGTCCTCCATTTTGAGCTCCCTGCAGCAGGGCC
GGGAAGCGGCCATCTTTCCGCTCACGCAACTGGTGCCGACCGGGCCAGCCTTGCCGCCCAGGGCGGGGCG
ATACACGGCGGCGCGAGGCCAGGCACCAGAGCAGGCCGGCGAGCTTGAGACTACCCCCGTCCGATTCTCG
GTGGCCGCGCTCGCAGGCCCCGCCTCGCCGAACATGTGCGCTGGGACGCACGGGCCCCGTCGCCGCCCGC
GGCCCCAAAAACCGAAATACCAGTGTGCAGATCTTGGCCCGCATTTACAAGACTATCTTGCCAGAAAAAA
AGCGTCGCAGCAGGTCATCAAAAATTTTAAATGGCTAGAGACTTATCGAAAGCAGCGAGACAGGCGCGAA
GGTGCCACCAGATTCGCACGCGGCGGCCCCAGCGCCCAGGCCAGGCCTCAACTCAAGCACGAGGCGAAGG
GGCTCCTTAAGCGCAAGGCCTCGAACTCTCCCACCCACTTCCAACCCGAAGCTCGGGATCAAGAATCACG
TACTGCAGCCAGGGGCGTGGAAGTAATTCAAGGCACGCAAGGGCCATAACCCGTAAAGAGGCCAGGCCCG
CGGGAACCACACACGGCACTTACCTGTGTTCTGGCGGCAAACCCGTTGCGAAAAAGAACGTTCACGGCGA
CTACTGCACTTATATACGGTTCTCCCCCACCCTCGGGAAAAAGGCGGAGCCAGTACACGACATCACTTTC
CCAGTTTACCCCGCGCCACCTTCTCTAGGCACCGGTTCAATTGCCGACCCCTCCCCCCAACTTCTCGGGG
ACTGTGGGCGATGTGCGCTCTGCCCACTGACGGGCACCGGAGCCTCACGCATGCTCTTCTCCACCTCAGT
GATGACGAGAGCGGGCGGGTGAGGGGGCGGGAACGCAGCGATCTCTGGGTTCTACGTTAGTGGGAGTTTA
ACGACGGTCCCTGGGATTCCCCAAGGCAGGGGCGAGTCCTTTTGTATGAATTACTCAACTAGTGATATCT
TAATTAACAAACGGCCGCTGCATTCTAGTTGTGGTTTGTCCAAACTCATCAATGTATCTTATCATGTCTG
GATCCCATCACAAAGCTCTGACCTCAATCCTATAGAAAGGAGGAATGAGCCAAAATTCACCCAACTTATT
GTGGGAAGCTTGTGGAAGGCTACTCGAAATGTTTGACCCAAGTTAAACAATTTAAAGGCAATGCTACCAA
ATACTAATTGAGTGTATGTTAACTTCTGACCCACTGGGAATGTGATGAAAGAAATAAAAGCTGAAATGAA
TCATTCTCTCTACTATTATTCTGATATTTCACATTCTTAAAATAAAGTGGTGATCCTAACTGACCTTAAG
ACAGGGAATCTTTACTCGGATTAAATGTCAGGAATTGTGAAAAAGTGAGTTTAAATGTATTTGGCTAAGG
TGTATGTAAACTTCCGACTTCAACTGTAGGGGATCCTCTAGGGCCGCCAGTGTGATGGATATCTGCAGAA
TTCGGCTTCAGGTACCGTCGACGATGTAGGTCACGGTCTCGAAGCCGCGGTGCGGGTGCCAGGGCGTGCC
CTTGGGCTCCCCGGGCGCGTACTCCACCTCACCCATCTGGTCCATCATGATGAACGGGTCGAGGTGGCGG
TAGTTGATCCCGGCGAACGCGCGGCGCACCGGGAAGCCCTCGCCCTCGAAACCGCTGGGCGCGGTGGTCA
CGGTGAGCACGGGACGTGCGACGGCGTCGGCGGGTGCGGATACGCGGGGCAGCGTCAGCGGGTTCTCGAC
GGTCACGGCGGGCATGTCGACAAGCCGAATTCCAGCACACTGGCGGCCGTTACTAGGTAGCTAGCTCGAG
CCTTCGAAGATCTCCTAGGGAAGTTCCTATACTTTCTAGAGAATAGGAACTTCGGAATAGGAACTTCACC
GGTGGGTGAAAAGCCGAATTCTGCAGATATCAAGCTTATCGATACCGTCGACCTCGAGGGGGGGCCCGGT
TAGATCCCCGGGTACCGAGCTCGAATTCATCTATGTCGGGTGCGGAGAAAGAGGTAATGAAATGGCATTA
TGGGTATTATGGGTCTGCATTAACGATAGCTAGCCTCTAACTCCTAGACCGTCAGAACTGCTGGGCCCTT
CAAGACGGGCTGCTCACACCCACTCATGTTAAGCCTGGTGAGGCCTGTACTCTGTTTTCACAGGAAGAAA
TCCTCACCCAGTCTTCCCCAAACACATTCCCAGGTTGTGTCATTAGTGGGATAGAGATGATTATTGTGGG
GAGAAGAGAAACATCTGGATGGATTTGGTTAGGTTGATCTATAGAGGAAGTAGGTGCTGCCTGAGGTAGC
TGTAATAGAAGCTAAAGGTCAAAGGAGAGGGCCCTGTCCCAATCCAGATGACTCCACTTCTGCTGGACCC
AGGTTCACAAGCTTAATCTACATTTCACCTAAATTTGGCTAACAAGCCCAAAATCACACAGGCAAAGGGA
GAAGTGGAGGCAGAACCGAGGTTGGAGGCCACCAGGGCCACCGGGCAGAGATCATTTAAGCCCAACCTTC
TCACTTCTCCCTGGGCTCTGCCTCTCTTAAAGGACCTTGTGGTGTGACCTCTTGTAGGTCCCTTTCACAC
TCGGGGCCTCAGTTTCCCCACTGTAAAGTGAATGGGTCCCAGCTTTGGTAAGCTTATGCTTACCTGATGC
TTTCTTCCTGGGCTGCTCTTGTAGAGAAAAGATAAATCTTCTTCCTCCATCCACGAGGGCTTCTTTCCCT
GGGGGTGAGAGTAGGCTGAGGAGAGCCACTTGCACACACTCTTAAAGAAAGTATTACCTGCACCAGCTCA
GTGAGAGGCACAGATCAGACTGTTACTTGAATCAAATTATGAGCCTCCCCAAATATATCTATGACATTTA
AATAGGGGATTACTTGAACATAGACTTTGGGATCCGGTGTGGAGTGCAGGAGACTAGCAAAGTGAATCCT
GAGAGTAGCAGGTCTGCACCTGTTGGATCGAGAAAGGCGGCCTACAATTCTGGTCAAATGAGCTGTGCTT
ATTGACATATTCTATTAGAGAGTACTACCAGGTCACCAGTCACCAGAAAGGCTGCCAGCTCTCCAACCAC
CTCCAGGGAACTATCCTGAATGGGGCCTTAACAAGCCTAAGAGAGGGTTGGTTTGGGTCCCAAGCCAATA
TTTGCTCTGCTTTATGTCAGTCATATGGAACCCAAACCAACCCTCTCCTATGTGCCTCACCAGTCGGTGC
AGGGATCCCAATTTCAAGTTTGGTTTTTTATGGTCAAAGTCCAGCATAGATTAAATGAAGGGGTGTGATG
ATGGTGTTAAAAGAGAACTCCAGACCAGTTTAACTCTTGGACACACATCCCATCTCACCATGGTGCTTCC
AACCTTCCAGAGATGATGGGCTCCTATTTTCTGATGACAAAGCCCTCCACAGGATTGCTGCCTGGCCATC
AGGGAGTGCCTCTGTAACTGAGGCTGAGATCCCACTTTCAGTCCTCCAGCTGTGGCCCATCCCTGCTCCG
CCCACCGGGTATGGCCTGTCCTAGGCTCTTAGGTATGGCTGCATTGTGAAATGATGGCTACAGAGCTGGC
ATCTCCTGTAGTCTGGTTCATCTAGTGCACTACCTCATAGTTAAAAGAAATCTGTTTAAGCCACTGAGGG
TGGCTCCTAGTGCCAACTCCAAGAACAGGAAGCTTCCCTTTTTTGGGAGGAGGGGCAGATGGTAACATGG
ATCGTCCAGGTCAATGGGAGCAGGGCAACCACAGTAAGTACTGGACAACAACACAAAACTCCATGTGTGG
CTTCCATCGAGTCCCTCTCCAATTGGTTTGGTCTTCTCCGTCCCATGCAGCACTTTAGCAAGGGGCCTGG
CTGAAGGCTATGAATTGTGTGGAGCCTCCTCATTGCAGTCTCCAACCATCTGATGCTGGGAAAATGTCAC
CAGGATGCAGCCATGCCGTGTGGCCAATGAACCGAGAAAACACCCCTTTTCTAGAATGCTCTAAAGAGGC
AGAATAATCCAGAGGTGAGGAAGGAAATACTCCACCAGAGACCCAGGCAGTTCCTACAAAAGCCAGACTT
TCCTTCACCTAGGGAGTGACAAGACCAGTGGAAAACACTCTCAAGCAGTAACCCCCAAATGCTCTGCAAG
CCAGTGGCGTCCAGATACCGCACAAGCGAGTGGGCTGTCTAATCCCATCATCATGATGTAAATATCTCTA
GGCTGCCCTGGGCTGTGCCTGACCCTGTCTTCAGCTTTCCACACCTCCACCTACAGCCCATGCACAGAAG
GACCACCCAGGAATGCTGCAAGTGTGGCACCTCCAGGGCCACCCAGGGAGAAGGAGGGCAGCTATGCTGG
TGGCTCCAGGGGTGGTACCTTCACACCACAAAGCCCAAACTGAGGCCCCAGATTTGGCTGATGAGGGCAT
ATTGGACAGGGGTCACTTATGCTCTTCCCCATTGCCACCTGGCCTCTGGCTACCTGGACTTGGCTACCTG
TGGATCCTCTCACAGGTGCCACCATCTTGGCTGAGTCTCCAGATGCGAGGTCCCTGAGGCAGTGGCAGGC
TTCTCGCTAATGCTGATGGGATTAGGAATGGGATAGGTGGGGAGGGCCCTGGACTGGGCCCTGATGAGCC
AAGTGGGTTTTTAGAGGGGCTACTGGTACATTTCAGGGACAGGACATCTGGTAGAGCTAAGCTGGGGCAA
TAAGGAGCCACTGCTAATCTGAGAGCTAGAAACAATCAGCTTCTGGGTCATTATTAATTAGGGTAGTTTG
GGCTGTGTGGAAGTCACGTACTATATGGGGTAGCCACAGCTCTCTCTACAGATAATCTCTAAGACTTCTG
ATTGGGACCGTGTGAATGCAGTAGCAATATCTCTTCTTACTGCCAGGCCCTGCCAGTCCTGCCTCCACGC
CCTGGCTGGCCCCCCTTATGATCTGACCCATGCCAGGCTGCCATAGTATGTTACTTCTGCATTAGCACTC
CTTGGGACCTGCCTCTCCACTGTCCCTCAGACTTTAAAGAACTATACAAACCCAAGGGGCTCTTCCCAAG
AGAATTGATATGACTTGAGGTGATTCCATTTCTGGAAGTAGTCACTCCATTTTCTGCCTCACTCTTTCAG
TGCTTCACAGAGCAGGTTCGAACGAAGGAGCCATCCAACTAACCGTCATGTTCGGGCAACCGAAGAAGGG
AGTGGCAGGATTTCCTTTGGAGACTTCTGGAATTAGACAGCAGTTTAATGCAAGCATCTAAATTCTCTTC
CTCCCAGAGTCTCATTAAAACTACAGTAAGAGTTTGTGTTTTGTTTTGTTTTTAAAGACAAAATCCCACC
AGGATAGAGAGAATAGGAGAGGAGATAACAGCATCATAATTTATGAAACTAAAATGCAGATAGACCAGGA
TTAACTGACTACACAGCACCAAGGAAGCTGAATCACAAGACAGCAGAGGAGAAAACTGGAAAGGATCGTG
GTCTATACGGCAGAATCTTCCCAAGCCTCAGGAGGAGGAGCTCTAGATGTTCCCAGATCTGGGAGGTAAA
GTGGAATGGGGGGACATGGTCAGCGTAATGGGGTTGGGCTGGAAGCTGGTTAAGGAGCAGGCAGATCTCT
GAATCCCCTCTCTGACTCTGTGTCCCCAGGCATCTGCCTGTCCCCCACCCTGGAAGAGGTCTGGCTTGAC
CCTTTGTCTGGTGAATTTCCTGCTCTGCTTTCCTGGTCCTGCTGGCCAGATCAGTGGAGGCCACTCACTT
CACCCCACAGGGATGTTCTGTGTTGCCCTACACCTGGGAACTGGAGGTACTGGAGGCAGGCTGTGGTGAG
CTTGAAAGCAAAACACAGAGGGCAGTCCAATCTCTTTGGCCATATTTCTTCTGCATATCCAATACCATGT
CCACAACTCTGCTAGTGTCCTGATGGTGGTGGGCTCTACACATTCCCGGGAAGCTGAAGGCAGATAATGA
CCAGGACAGGTCAACCTCTCTTCTTCTGAAAGCCTTCATCTACTAATGGCCTGGGACTCTTCCCTTAAAT
GCTTAGATTGTGTCTTCCACTAAGGTTTTTTGCTGTTGCTGTTGTTTGTTTGTTTGTTTGTTTGTTTGTT
TGTTTTGAGACGGAATCTCACTCTGTCGCCCAGGCTGGAGTGTAGTGGCACAATCTCAGCTCACCACAAC
CTTCACCTCCTAGGTTGAAGGGATTCTCCTGCCTCAGCCTCCTGAGTAGCTAGGATTACAGGCACATGCC
ACCATGCCTGGCTAATTTTTGTATTTTTAGTAGAGACAGGATTTCGCCATGTTGGCCAGGCTGGTCTTGA
ACTCCTGACCTCAGGTGATCTGCCTACCTTGGTCTCCCAAAGTGCTGGGATTACTGGTGTGAGCCACCAC
ACCCGGCCAAGGTTTTTGTTTGTTTGTTTGTTTGTTTGTTTTGTATTGAGGCAGGGTATCACTCTGGTCA
CCCAGGCTGGAGTGCAGTAGTGCAATCACGGCTCACTGAAACCTCCACCTCCCTGGCGGGCTCAGGTGAT
CCTGCCACCTCAGCTTCCCAGGTAGCTGGGACTACAGGCTTGTACCACCACTCCCAGCTAATTTTTGCGT
TTTTAGTAGAGACAGGGTTTCCCCATGTTGCCCAGGTTGGTCTCAAACTCTGGGCTCAAGCGATCTGCCT
GCCTCAGCCTCCCAAAGTGCTGGGATTACAGGTGTAAGCCACCGTACCCGGCCCCGCCACTAAGGTTTTG
AAAATGAAGCAATTACAAGTTTAAGTCTATTAATAAGTGATGAAGCTATGTAGAAAAGCAGAATAATTAT
CTTGGATCAGGAAGGTCACATGAGGATCTACTTGGGGGTTGTCAATATTCTATTTCTTGACCTGATCAGT
GTTGACAGCAGGTTTTAATTTTTTACTTCTTTTTGTTTGTTTGTTTTTGAGACGGAGTCTTGCTCTGTCT
CCCAGGCTGGAGTGCAGTGGTATGATCTCGGCTCACTGCAACCTCCGCCTCCTGGGTTCAAGCTGTTCTC
CTGCCTCAGCCTCCCCAGTAGCTGGGATTACAGGCAGGCACCACCACGACCAGCTAATTTTTGTATTTTT
AGTAGAGACTGGGTTTCACCATCTTGGCCAGGCTGGTCTCGAACTTCTGATCTCGTGATCCGCCCTCCTT
GGCCTCCCAAAGTGCTGGGATTACAGGCTTGAGCCAGCGTGCCCGGCCCATTTTTTACTTCCTTATTAAA
CTGTACATATAGGCCTTGCACACTTTTCTGCATCAATGTTATATTCCACAATAAAGGGAAAAGGTATATA
CACAACTTGATACCAGTAATGTGAAACATATATTTCTACATAGAAAAAAAAATGACTGAAATACTGCACT
CCAATGTGTTCACACAGTAGTTGTTTCTGGATTATTTATATATTAAATGTTTATATATTGTATTATGCCA
TGAGGTTTGTGTTTTCTCTCCACTTTTCTGCATTTTCCAAGTTTACTACAAAGAGCACATATTACTCTTA
TAATCAGAAAGTCATAAAATATATTTAAAAAGACAAAATTGAAACTAATAAGGATCAACACAAAACAGAT
GAGCCATCTGTGGAAATCCGCACAGAATACTACCTAAAGAGATTGGTGACGTGCATGATCTCACTAGGAT
GAGCACAAAGCTTGCCAGAGCCTAGGGTCTATTTCTAGGGTTGGCTCTTGGAAGCCAGGATAGTTGTTAT
CTCTGGGAAGAGGGAGGGGCACACAAGGGGCTTCTAAAACATTCTGAATGTTCTATTTCTGAACCTGGTT
GGTGGGTACATGACTGTTGGTTTTATTATTATATGTTTTATATACTCTTCCGTATGTATGGTGTGGATTC
CAAAAAAAGATTTCCTTTAGAGAAAACCAGAATCACATAAGTAGAAAATATGGTGCTATGTTGAAGGAAC
AACTCAAGTTTATATAAAATCATCATCATTTATAGGCTTAAAAAGTTGCTTTGGAATTTTGGTCTAACTG
ACTTGTCTTTTCTGCAGCAAACCACGCTCCTTCTGGACGTGCTCCAGGCAGAGGGGATTAGGGTGGGTTC
AAGGCTGCAAGTACCTAGCTCAGCACACTCTCTTCAGGGGACTTAGAGTTTGTCTGGTGTTGGCTCTCTG
AGCTCTTGTCAGGAATGCCGACCCTTCCGAGGTTCAGGATTTGAAGCCTGCCTTCCCACCCCAGATTTGG
TCCACACAGACACTCAAGTATGTATTTCAACTACAAATGACCTGTACTTTCCTATTACTCCTCTCTTTCA
TGGTAACCTTTCTGGTATCCTTCCTTCCCTACATTTATGGGAGGGGGACATCATTCTCTGCTCTCCTGTC
ACTGAAGGCTCCACCTTCTGTCTTCTTCTGACCCATCTGGTTTTCCTGGGGCCACCTCCTCTCCTTACCA
CCCTAACGCTTTTGTAACTTGAGGAGAAATGAGAGATCACCTAGTCAGGTCATCATTCTCTGTAGATGAA
GAGGCCCAATGGTTTGCTCAAGAATTGCCAAGCGAGTTAAAGACAGAGAGTATGAGAGTCAGCAAGACCT
ACAGAAAGCATCTATCTGCACTGTTTTGCAGGGACTTAGCCTTTGTGTGTGGACTCCTGGAATGCCACCC
ACTAAGAAACATTGTCTGACACCAACTCCCCACTTGGTAGGTGGGGACACTGAAACTCATGGCAGGAAAG
GGCCTTGCCCCAAGCCAGGGCAGAGTGTCACTCATCACTCTCAATTTTCAGTCCAGGGCACCTTGTTGTG
ACTATCCCAAAGGCAGCCACTTTCCCTGGTCTGAAAGACCTGAAGAGAGAAGAGAAGAGAAGGATGGAAG
GCAGAGTATGCGGCTTTGATTCATTTCCTGGTGAAAACAGATCTATACGAGAAGCAAATTTCACGAAAGG
GAAGAGAAGAAAGTGTCCCATACGTTGCTGGCCTGTTTCAACCTTGCTTTGATTCTTGCTGAAAAGGGTA
CCGTGTATTTCTGAGTTCAACATGCAGACCAGTGTTAGGAAAGCCACTGCACCTCCACTTTAGCCTCCAG
GGCTGTGCCCTGCAAATGGCCTGCAGCCTTGGTGCCTCGCTCTCCAGACTGCATTTTGGAAGATGGGACA
GAGGCTTATGGAAGCCCACATTAGAACGGGGGAGCAGAATGGGTGAGATGAGGGATCCTTGATAGTGAAC
CAGATGAAGGAATGGTAGCCAAATGCCAGGCCTCCTTTGTGGCTTCAATCCAAAGGCTCTGGAGCCCTTC
CAGGGCAGAACATCAGGCATGTTTACCCCCACTGTCCTCAACAGTGACAGAGGTGCAATCTTGGGCAGCT
GGCCATTTTGAAAGCAACCTCCTTAATCTCAACTGGGAAGGCTCCCTAGCAGGACCCCTGTGTTGCACAC
CTGGAGGAAGCTAGACTAACCAGAAGCTCAGCACGGTTCCATCTGGGATGCCCAGGTCTGAGACGAAAAA
GGTAACTCTCTTTTCTGGGTCCTGGCCCAGTTGTGTCTCTCTCCACCTCATTCTCTGAGATGCCTGTCTC
CCCTTTTTTGTCCCATCAGGAGGCAAGAGCTATCACTGGGCCAGACTCCACCAGAAGCCAAGCCAGCTTG
TTACCCAGCTTCTCAGGGAGCAAAGAACAGCCTTGTTTCTATCTTATCCCCACTGTCCCCTGCCCCTGCC
CCACCTCCCAGCCATTCAGCTTCTGGCTTCCCCAGAGCTGCCTGCTTCTTTGTGGTCCTCCATTCCTTGA
AAAGACCTTCTAGTCATTAGTGTATATAAATGGCCACTTAGCCCAGATTACAGTGAGGTCAACAGCTGGG
GCTCTGAGAATTGTCACACACTGGCACAGGAGAGGAGGCTATTCTTCCAGAGAATTTGGAGGGCACTCCC
ATCCACTTACAACAAAAAGCCCATCCACTGTGCTTGGCAGTAGGTGATCTGAGAACCAATGGAACCAGGT
TAATCCTGTGGCACTGTTGAGTGAGGAGAGCAGTGGCGGGCACTGGAAAATATCAGAGACAAGGCAGGAG
ACCTGAAATCTAGGCTTAGCTCCTCATATACTTGGCAGCTGTATGACCTCAGACAACCAGTGTTACCTCT
CTAAGCCTCAGTTTCCTCATGCAAAAGGAGGGGGAATAACAACAGAGCCCACTGCTTGGGGGTGTTGTGA
GGACAGGATGAAAAAACAAACAGAAATCCCTCAGTACAGGATTCAGTGCAGTGGACAGTCTTGCAAGGTC
TGGTTCAGCCCTCCACCCCTACCCTCACCAGTATAAAGAACTCTGGCCTACAAGTCAGATGACCTGAGTT
TTAATCTCAGCTTTGCCATTAGCCGTGTGAACTTGAGAAAGTCCCTTTCCTTTTTACATCTATTGGGATG
ATCATGCATTTTTTGTCCTTTATTCTGTTAATATAGTGTGTTACATTGATTGCTTTTCATAGACTGAACC
AGCCTTGTATTCCAGGGATAAATCTCACTTGGTCATGGTGTATAATCCTTTATACAAATGTTGCTGGGTT
GAGTTTGCTAGTATTTTGTTGAAGATTTTTATGTCTTGATTCATAAGGAATATTGGTGTACCTTCCCCTT
TTATGGCCACAGTTTCCCTACAATGATGTAGTCGAACTAGACAACCTCCAATATCTTTCAGTATTCATGT
CCTCTGATTCTGTGAAACTAAGAAAATTAAGAAATAGTGATTCATAGGCACAAGGCAGGCAAAACTTAGA
CTCCTTGTAGAATAATTAGGAAGCCAAATATTCAGTGTGCTTATTTCTCAAATAACCTTAGTTTCTCCAG
TCTGCCCCAACTCCGAGGCCTGAATATCTCTAGATGCTTATGATGGCAACTAAAGCCTAAAAGCTAATTC
ATTTTAAAGTTCTTCCAAATGCATAGGGTTTTATTTTTCCAGACCTGGGTTCAGATGGGGAATTTGACAA
ACAATGGAAAGGGGGAAAAACAACAATCTAAACACTGAGTGACAAAGTAACAAAGAAATAGTCTAGCTAT
CAGCCAGTCAAGCCAGCCTTGGCTTTGCTATCCAAAGTAGTCAGTCTAATTCTACCACCAGTTTCTGTTC
CTGTAGCTGTCTACTGCCTGCCAGGGACTCTGCCTTCCCACCCACAACTACCAATGGAAGGATGTGGTGA
CCATACCAGTGGCTGCTGACATCTCCTGCCATGGGAAGCATAATTGCCTCCAGCAGCCTCCCCCTTAGAT
CCATCATTTTTGTTGCACTTGGCCTGGGCTGTACTCCCGGCCAATGACTGAACATGGTGAGCATAGTAAT
GCAGGCCCATTTCTGTGAGGAGCAGGACTCCTCCAGTAGGTGACTTTGGCTCAAGGACTCTCTATTGGCC
TGGTTGAACTTTTCCTGAACTGTGCTACTGTCTGAGACTCTTCTTACCCAATCCTCTTTCTCGCCCCAAT
TGTCACAGACCACCTGCATTGTGGTCTGAGTCTCTCCCCACCTTCTCTTGCTCTTCCCTGTTTATCTTTC
ACAGGCATTTCCCCCAGTACATTCCTTGAATGTCTAACCCGATACGGGTGCCTGACTTTTGGCAGACCTA
AGCAGACAAAAAGGAGTACTTGGTTACCTAGCTCTTCTTTCTACCACAAACATCGAGGGAACCCTTTTTC
CCTCACCCCTCTGCCACACCCCCACTGCCCCAGTGAACAACCACAGAGAGAGCTGTGGTATAATATTAGG
CTGGTGCAAAAGTAATTGCGGTTTTTGCCATTACTTTTAATGGTAAAAACCGCAATTACTTTTGCACCTA
CCTAGTATTTGTGTCCCCCCAAATTCATATGTTGAAACCTAACCCACAATATGATGTCATTAGGAGGCAA
GACCTTGAGGAGGTGATTAGATGATGGGGTGGAGCTCTCCTGAATGAGATTAGTGCCCTTATAAGAAGAA
GCCCAAGGAAGCTACCTTGACTCTTCCATCACATGAGAATGCAGCAAGAAGGCACCATCTACTAATCAGG
AAGAGAGCTCTCACCAGACACTGAATCTGCCAGTGTCTTGATCTTGAAGTTCCCAGCCTCCAGAACTATG
CATAATGCATTTCCATTGTCTCTAAGCCACCCAGCCTATGGTATTTTGTCATAGCAGCCTGAACTGACTA
AGACAGTGAGCCACATGAGAAGTGCCCCAACCCCTCCCTTAAGCACTTGGCTCACAGATCAGTGGGTTCA
TTTCTGCCTGAGTTTTATTGTTATTCTGTAGATTTCTTGGGCTAGATATATTTTTCTGTTATTTTCCTTC
TTCACCTCAGTCATGAATTGGTTGTTTTAAAAAAGACAATGTAAGTCATGGGGAAACTCCTGACAACTCT
ACTCTCCTAGGGTTCCTGATAAAAGGGGATTCAGTTGAGTCCTCTGATGGTCTCTACCTGCCAAAGTCCA
GCAGCCCTTAGCAAACATGCTGCTCGTTTCTGTAGAGAAGGTGCTGGTGTCCCACCATACTTCTCTCTCC
CTCATGAAGGGCTTGCGACCCAGCAAATGGGTGGCTTATATGGGTCTGTTTCAAAGGAAGAGCCAGCTCT
GGGAAGAAAAACGATGAGCATAAGCATAACCTACCACTGTGCCTGGGAAAGCAGACAACTTTTTTGATGT
GTGAATATCTAATGAGAATGGAATCCATCAATTACCTTAAACTTAGGCACAGTCTTCAAATTCAATATAT
GTGGGATATACTTTTAGTCAGTTTGTAGACGTTATTTGTAATAAATAATCTGGCTTCTCTAAAGAAATTA
TTTTAAGTGTTTGGTTTGGTTTGATTTAATGGTAAAATTATATTTAGTGGCAGAGAATTATAGCAATGGT
GATAAACTATAGAGTGTCATAAGTTCATATCTTATTCTCACATTTGAAGCTGCCTGCAGATGCATTCAAG
ATGCAGCCAGAAGTCAGGAGACTCAGGCTGTTATTTGGAGCTCATCATTTTACAGCCTTGCTGGACTCCC
ACTTTCTCAGGGGAAAAATGTGGTGTTGACCCAGATTAGCTCTCCAGGCCCTGCTGAGTTGGGCACTCTG
TAAGCTGGAGGGTCTTCTATTGTCTTCACCTAAGTGTCAATCAACAACCCAAATGGGCATGGGGGAAGAG
GGAGCTGGGCCAATGCCCAGGGTGCCTGGTAGAGAGATACCTTGGGCACTGGAAGGCACCAGCTTCCCAG
AGAGAAGGGGGAGGGCCATGAAAAAGTTGGCTGTAGATGCCAGGGACACTGGGACTCTCCAGCTGTGTGT
TTGTGCCTTCTGAAGACTTATGTTTCATTCCTTTGGAGCATGCATAATCATACACTGTGGGATGTGTTAT
ATAGATTGCTTGATAGTTCACCACTGTAATAAAATACTGTGACTGGAATCTGCTCCCAGTCTGCCTTTGA
TAGCACTTGTGCAACACACATTTACTGAGCATTTACAGTGATCCAGGACCTGTGTTGTGAAAACATTGAT
GGACAAGGCAGATGGTGGAGCACGTCAGTGAGGATTTTTAACAAAGGCTGGTAAGTGCTATAAAGGAACA
TTGTAGGACACTAGAGAACAAAGAACAGGAGAACCTGACTTAGGCTGGGGTGGGGCGTTGGTTAGAGGAG
GCTCCTTGGAGGACATGAGGTTTAAGCTGTGACCTGAGGATGAATAGATGTTGGCCAGTGAGGTTACCTC
AAATCGTCACTTCCGTTTTCCCACGTTACGTCACTTCCCATTTTAAGAAAACTACAATTCCCAACACATA
CAAGTTACTCCGCCCTAAAACCTACGTCACCCGCCCCGTTCCCACGCCCCGCGCCACGTCACAAACTCCA
CCCCCTCATTATCATATTGGCTTCAATCCAAAATAAGGTATATTATTGATGATG (SEQ ID NO:
179)
The nucleotide sequence of WL-PS1

| | |
|---|---|
| 1-->178 | Start: 2582 End: 2759 |
| loxP | Start: 2768 End: 2801 |
| 179-->344 | Start: 2808 End: 2973 |
| loxP | Start: 2974 End: 3007 |
| 3112-->27435 | Start: 3016 End: 27338 |
| lambda-1 | Start: 27393 End: 29862 (Complementary) |
| BGH polyA | Start: 30176 End: 30390 |
| copGFP | Start: 30415 End: 31080 (Complementary) |
| CMV | Start: 31127 End: 31779 (Complementary) |
| lambda-2 | Start: 31831 End: 33360 |
| 30544-->31879 | Start: 33421 End: 34756 |
| Ad5E4orf6 | Start: 34752 End: 35866 |
| 32972-->34794 | Start: 35864 End: 37686 |

taaacttggcgcgccctgagtgatttttctctggtcccgccgcatccataccgccagttgtttaccctcacaac
gttccagtaaccgggcatgttcatcatcagtaacccgtatcgtgagcatcctctctcgtttcatcggtatcatt
acccccatgaacagaaatcccccttacacggaggcatcagtgaccaaacaggaaaaaaccgcccttaacatggc
ccgctttatcagaagccagacattaacgcttctggagaaactcaacgagctggacgcggatgaacaggcagaca
tctgtgaatcgcttcacgaccacgctgatgagctttaccgcagctgcctcgcgcgtttcggtgatgacggtgaa
aacctctgacacatgcagctcccggagacggtcacagcttgtctgtaagcggatgccgggagcagacaagcccg
tcagggcgcgtcagcgggtgttggcgggtgtcggggcgcagccatgacccagtcacgtagcgatagcggagtgt
atactggcttaactatgcggcatcagagcagattgtactgagagtgcaccatatgcggtgtgaaataccgcaca
gatgcgtaaggagaaaataccgcatcaggcgctcttccgcttcctcgctcactgactcgctgcgctcggtcgtt
cggctgcggcgagcggtatcagctcactcaaaggcggtaatacggttatccacagaatcaggggataacgcagg
aaagaacatgtgagcaaaaggccagcaaaaggccaggaaccgtaaaaaggccgcgttgctggcgtttttccata
ggctccgcccccctgacgagcatcacaaaaatcgacgctcaagtcagaggtggcgaaacccgacaggactataa
agataccaggcgtttccccctggaagctccctcgtgcgctctcctgttccgaccctgccgcttaccggatacct
gtccgcctttctcccttcgggaagcgtggcgctttctcatagctcacgctgtaggtatctcagttcggtgtagg
tcgttcgctccaagctgggctgtgtgcacgaaccccccgttcagcccgaccgctgcgccttatccggtaactat
cgtcttgagtccaacccggtaagacacgacttatcgccactggcagcagccactggtaacaggattagcagagc
gaggtatgtaggcggtgctacagagttcttgaagtggtggcctaactacggctacactagaaggacagtatttg
gtatctgcgctctgctgaagccagttaccttcggaaaaagagttggtagctcttgatccggcaaacaaaccacc
gctggtagcggtggtttttttgtttgcaagcagcagattacgcgcagaaaaaaaggatctcaagaagatccttt
gatcttttctacggggtctgacgctcagtggaacgaaaactcacgttaagggattttggtcatgagattatcaa
aaaggatcttcacctagatccttttaaattaaaaatgaagttttaaatcaatctaaagtatatatgagtaaact
tggtctgacagttaccaatgcttaatcagtgaggcacctatctcagcgatctgtctatttcgttcatccatagt
tgcctgactccccgtcgtgtagataactacgatacgggagggcttaccatctggccccagtgctgcaatgatac
cgcgagacccacgctcaccggctccagatttatcageaataaaccagecagecggaagggccgagcgcagaagt
ggtcctgcaactttatccgcctccatccagtctattaattgttgccgggaagctagagtaagtagttcgccagt
taatagtttgcgcaacgttgttgccattgctgcaggcatcgtggtgtcacgctcgtcgtttggtatggcttcat
tcagctccggttcccaacgatcaaggcgagttacatgatcccccatgttgtgcaaaaaagcggttageteette
ggtcctccgatcgttgtcagaagtaagttggccgcagtgttatcactcatggttatggcagcactgcataattc
tcttactgtcatgccatccgtaagatgcttttctgtgactggtgagtactcaaccaagtcattctgagaatagt
gtatgcggcgaccgagttgctcttgcccggcgtcaacacgggataataccgcgccacatagcagaactttaaaa
gtgctcatcattggaaaacgttcttcggggcgaaaactctcaaggatcttaccgctgttgagatccagttcgat
gtaacccactcgtgcacccaactgatcttcagcatcttttactttcaccagcgtttctgggtgagcaaaaacag
gaaggcaaaatgccgcaaaaaagggaataagggcgacacggaaatgttgaatactcatactcttcctttttcaa
tattattgaagcatttatcagggttattgtctcatgagcggatacatatttgaatgtatttagaaaaataaaca
aataggggttccgcgcacatttccccgaaaagtgccacctgtctagctacgatatcctgtttaaacatcatcaa
taatataccttatagatggaatggtgccaatatgtaaatgaggtgattttaaaaagtgtgggccgtgtggtgat
tggctgtggggttaacggttaaaaggggcggcgcggccgtgggaaaatgacgttttatgggggtggagtttttt
tgcaagttgtcgcgggaaatgatttaaatataacttcgtatagcatacattatacgaagttatggatccttacg
cataaaaaggcttcttttctcacggaactacttagttttcccacggtatttaacaggaaatgaggtagttttga
ccggatgcaagtgaaaattgctgattttcgcgcgaaaactgaatgaggaagtgtttttctgaataatgtggtat
ttatggcagggtgataacttcgtatagcatacattatacgaagttatatttaaataggaatgtttatgccttac
cagtgtaacatgaatcatgtgaaagtgttgttggaaccagatgccttttccagaatgagcctaacaggaatctt
tgacatgaacacgcaaatctggaagatcctgaggtatgatgatacgagatcgagggtgcgcgcatgcgaatgcg
gaggcaagcatgccaggttccagccggtgtgtgtagatgtgaccgaagatctcagaccggatcatttggttatt
gcccgcactggagcagagttcggatccagtggagaagaaactgactaaggtgagtattgggaaaactttggggt
gggattttcagatggacagattgagtaaaaatttgttttttctgtcttgcagctgacatgagtggaaatgcttc
ttttaaggggggagtcttcagcccttatctgacagggcgtctcccatcctgggcaggagttcgtcagaatgtta
tgggatctactgtggatggaagacccgttcaacccgccaattcttcaacgctgacctatgctactttaagttct
teacctttggaegeagetgcageegetgcegeegcctctgtcgcegetaacactgtgettggaatgggttacta
tggaagcatcgtggctaattccacttcctctaataacccttctacactgactcaggacaagttacttgtccttt
tggcccagetggaggctttgacccaacgtctgggtgaactttctcageaggtggccgagttgcgagtacaaact
gagtctgctgtcggcacggcaaagtctaaataaaaaaaattccagaatcaatgaataaataaacgagcttgttg
ttgatttaaaatcaagtgtttttatttcatttttcgcgcacggtatgccctggaccaccgatctcgatcattga
gaactcggtggattttttccagaatcctatagaggtgggattgaatgtttagatacatgggcattaggccgtct
ttggggtggagatagetecattgaagggatteatgetccggggtagtgttgtaaatcacccagtcataacaagg
tcgcagtgcatggtgttgcacaatatcttttagaagtaggctgattgccacagataagcccttggtgtaggtgt
ttacaaaccggttgagctgggaggggtgcattcgaggtgaaattatgtgcattttggattggatttttaagttg
gcaatattgccgccaagatcccgtcttgggttcatgttatgaaggactaccaagacggtgtatccggtacattt
aggaaatttatcgtgcagcttggatggaaaagcgtggaaaaatttggagacacccttgtgtcctccgagatttt
ccatgcactcatccatgataatagcaatggggccgtgggcagcggcgcgggcaaacacgttccgtgggtctgac
acatcatagttatgttcctgagttaaatcatcataagccattttaatgaatttggggcggagcgtaccagattg
gggtatgaatgttccttcgggccccggagcatagttcccctcacagatttgcatttcccaagctttcagttctg
agggtggaatcatgtccacctggggggctatgaagaacaccgtttcgggggcgggggtgattagttgggatgat
agcaagtttctgagcaattgagatttgccacatccggtggggccataaataattccgattacaggttgcaggtg
gtagtttagggaacggcaactgccgtcttctcgaagcaagggggccacctcgttcatcatttcccttacatgca
tattttcccgcaccaaatccattaggaggcgctctcctcctagtgatagaagttcttgtagtgaggaaaagttt
ttcagcggttttagaccgtcagecatgggcattttggaaagagtttgetgcaaaagttctagtctgttccacag
ttcagtgatgtgttctatggcatctcgatccagcagacctcctcgtttcgcgggtttggacggctcctggagta
gggtatgagacgatgggcgtccagegetgccagggttcggtccttccagggtctcagtgttcgagtcagggttg
tttccgtcacagtgaaggggtgtgcgcctgcttgggcgcttgccagggtgcgcttcagactcattctgctggtg
gagaacttctgtcgcttggcgccctgtatgtcggccaagtagcagtttaccatgagttcgtagttgagcgcctc
ggctgcgtggcctttggcgcggagcttacctttggaagttttcttgcataccgggcagtataggcatttcagcg
catacagcttgggcgcaaggaaaatggattctggggagtatgcatccgcgccgcaggaggcgcaaacagtttca
cattccaccagccaggttaaatccggttcattggggtcaaaaacaagttttccgccatattttttgatgcgttt
cttacctttggtctccataagttcgtgtcctcgttgagtgacaaacaggctgtccgtatctccgtagactgatt
ttacaggcctcttctccagtggagtgcctcggtcttcttcgtacaggaactctgaccactctgatacaaaggcg
cgcgtccaggccagcacaaaggaggctatgtgggaggggtagcgatcgttgtcaaccagggggtccaccttttc
caaagtatgcaaacacatgtcaccctcttcaacatccaggaatgtgattggcttgtaggtgtatttcacgtgac
ctggggtcccegetgggggggtataaaagggggcggttctttgetcttcctcactgtcttccggategetgtcc
aggaacgtcagctgttggggtaggtattccctctcgaaggcgggcatgacctctgcactcaggttgtcagtttc
taagaacgaggaggatttgatattgacagtgccggttgagatgcctttcatgaggttttcgtccatttggtcag
aaaacacaatttttttattgtcaagtttggtggcaaatgatccatacagggcgttggataaaagtttggcaatg
gatcgcatggtttggttcttttccttgtccgcgcgctctttggcggcgatgttgagttggacatactcgcgtgc
caggcacttccattcggggaagatagttgttaattcatctggcacgattctcacttgccaccctcgattatgca
aggtaattaaatccacactggtggccacctcgcctcgaaggggttcattggtccaacagagcctacctcctttc
ctagaacagaaagggggaagtgggtctagcataagttcatcgggagggtctgcatccatggtaaagattccegg
aagtaaatccttatcaaaatagctgatgggagtggggtcatctaaggccatttgccattctcgagctgccagtg
egegetcatatgggttaaggggactgccccagggcatgggatgggtgagagcagaggcatacatgccacagatg
tcatagacgtagatgggatcctcaaagatgcctatgtaggttggatagcatcgcccccctctgatacttgctcg
cacatagtcatatagttcatgtgatggcgctagcagccccggacccaagttggtgcgattgggtttttctgttc
tgtagacgatctggcgaaagatggcgtgagaattggaagagatggtgggtctttgaaaaatgttgaaatgggca
tgaggtagacctacagagtctctgacaaagtgggcataagattcttgaagcttggttaccagttcggcggtgac
aagtacgtctagggcgcagtagtcaagtgtttcttgaatgatgtcataacctggttggtttttcttttcccaca
gttcgcggttgagaaggtattcttcgcgatccttccagtactcttctagcggaaacccgtctttgtctgcacgg
taagatcctagcatgtagaactgattaactgccttgtaagggcagcagcccttctctacgggtagagagtatgc
ttgagcagcttttcgtagcgaagcgtgagtaagggcaaaggtgtctctgaccatgactttgagaaattggtatt
tgaagtccatgtcgtcacaggctccctgttcccagagttggaagtctacccgtttcttgtaggcggggttgggc
aaagcgaaagtaacatcattgaagagaatcttaccggctctgggcataaaattgcgagtgatgcggaaaggctg
tggtacttccgctcgattgttgatcacctgggcagctaggacgatttcgtcgaaaccgttgatgttgtgtccta
cgatgtataattctatgaaacgcggcgtgcctctgacgtgaggtagcttactgagctcatcaaaggttaggtct
gtggggtcagataaggcgtagtgttegagageecattcgtgcaggtgaggatttgcatgtaggaatgatgacca
aagatctaccgccagtgctgtttgtaactggtcccgatactgacgaaaatgccggccaattgccattttttctg
gagtgacacagtagaaggttctggggtcttgttgccatcgatcccacttgagtttaatggctagatcgtgggcc
atgttgacgagacgctcttctcctgagagtttcatgaccagcatgaaaggaactagttgtttgccaaaggatcc
catccaggtgtaagtttccacatcgtaggtcaggaagagtctttctgtgcgaggatgagagccgatcgggaaga
actggatttcctgccaccagttggaggattggctgttgatgtgatggaagtagaagtttctgcggcgcgccgag
cattcgtgtttgtgcttgtacagacggccgcagtagtcgcagcgttgcacgggttgtatctcgtgaatgagctg
tacctggcttcccttgacgagaaatttcagtgggaagccgaggcctggcgattgtatctcgtgctcttctatat
tcgctgtatcggcctgttcatcttctgtttcgatggtggtcatgctgacgagcccccgcgggaggcaagtccag
acctcggcgcgggaggggcggagctgaaggacgagagcgcgcaggctggagctgtccagagtcctgagacgctg
cggactcaggttagtaggtagggacagaagattaacttgcatgatcttttccagggcgtgcgggaggttcagat
ggtacttgatttccacaggttcgtttgtagagacgtcaatggcttgcagggttccgtgtcctttgggcgccact
accgtacctttgttttttcttttgatcggtggtggctctcttgcttcttgcatgctcagaagcggtgacgggga
cgcgcgccgggcggcagcggttgttccggacccgggggcatggctggtagtggcacgtcggcgccgcgcacggg
caggttctggtattgcgctctgagaagacttgcgtgcgccaccacgcgtcgattgacgtcttgtatctgacgtc
tctgggtgaaagctaccggccccgtgagcttgaacctgaaagagagttcaacagaatcaatttcggtatcgtta
acggcagcttgtctcagtatttcttgtacgtcaccagagttgtcctggtaggcgatctccgccatgaactgctc
gatttcttcctcctgaagatctccgcgacccgctctttcgacggtggccgcgaggtcattggagatacggccca
tgagttgggagaatgcattcatgcccgcctcgttccagacgcggctgtaaaccacggccccctcggagtctctt
gcgcgcatcaccacctgagcgaggttaagctccacgtgtctggttaagaccgcatagttgcataggcgctgaaa
aaggtagttgagtgtggtggcaatgtgttcggcgacgaagaaatacatgatccatcgtctcagcggcatttcgc
taacatcgcccagagcttccaagcgctccatggcctcgtagaagtccacggcaaaattaaaaaactgggagttt
cgcgcggacacggtcaattcctcctcgagaagacggatgagttcggctatggtggcccgtacttcgcgttcgaa
ggctcccgggatctcttcttcctcttctatctcttcttccactaacatctcttcttcgtcttcaggcgggggcg
gagggggcacgcggcgacgtcgacggcgcacgggcaaacggtcgatgaatcgttcaatgacctctccgcggcgg
cggcgcatggtttcagtgacggcgcggccgttctcgcgcggtcgcagagtaaaaacaccgccgcgcatctcctt
aaagtggtgactgggaggttctccgtttgggagggagagggcgctgattatacattttattaattggcccgtag
ggactgcgcgcagagatctgatcgtgtcaagatccacgggatctgaaaacctttcgacgaaagcgtctaaccag
tcacagtcacaaggtaggctgagtacggcttcttgtgggcgggggtggttatgtgttcggtctgggtcttctgt
ttcttcttcatctcgggaaggtgagacgatgctgctggtgatgaaattaaagtaggcagttctaagacggcgga
tggtggcgaggagcaccaggtctttgggtccggcttgctggatacgcaggcgattggccattccccaagcatta
tcctgacatctagcaagatctttgtagtagtcttgcatgagccgttctacgggcacttcttcctcacccgttct
gccatgcatacgtgtgagtccaaatccgcgcattggttgtaccagtgccaagtcagctacgactctttcggcga
ggatggcttgctgtacttgggtaagggtggcttgaaagtcatcaaaatccacaaagcggtggtaagcccctgta
ttaatggtgtaagcacagttggccatgactgaccagttaactgtctggtgaccagggcgcacgagctcggtgta
tttaaggcgcgaataggcgcgggtgtcaaagatgtaatcgttgcaggtgcgcaccagatactggtaccctataa
gaaaatgcggcggtggttggcggtagagaggccatcgttctgtagctggagcgccaggggcgaggtcttccaac
ataaggcggtgatagccgtagatgtacctggacatccaggtgattcctgcggcggtagtagaagcccgaggaaa
ctcgcgtacgcggttccaaatgttgcgtagcggcatgaagtagttcattgtaggcacggtttgaccagtgaggc
gcgcgcagtcattgatgctctatagacacggagaaaatgaaagcgttcagcgactcgactccgtagcctggagg
aacgtgaacgggttgggtcgcggtgtaccccggttcgagacttgtactcgagccggccggagccgcggctaacg
tggtattggcactcccgtctcgacccagcctacaaaaatccaggatacggaatcgagtcgttttgctggtttcc
gaatggcagggaagtgagtcctatttttttttttttttgccgctcagaatgcatcccgtgctgcgacagatgcg
cccccaacaacagcccccctcgcagcagcagcagcagcaaccacaaaaggctgtccctgcaactactgcaactg
ccgccgtgagcggtgcgggacagcccgcctatgatctggacttggaagagggcgaaggactggcacgtctaggt
gcgccttcgcccgagcggcatccgcgagttcaactgaaaaaagattctcgcgaggcgtatgtgccccaacagaa
cctatttagagacagaagcggcgaggagccggaggagatgcgagcttcccgctttaacgcgggtcgtgagctgc
gtcacggtttggaccgaagacgagtgttgcgagacgaggatttcgaagttgatgaagtgacagggatcagtcct
gccagggcacacgtggctgcagccaaccttgtatcggcttacgagcagacagtaaaggaagagcgtaacttcca
aaagtcttttaataatcatgtgcgaaccctgattgcccgcgaagaagttacccttggtttgatgcatttgtggg
atttgatggaagctatcattcagaaccctactagcaaacctctgaccgcccagctgtttctggtggtgcaacac
agcagagacaatgaggctttcagagaggcgctgctgaacatcaccgaacccgaggggagatggttgtatgatct
tatcaacattctacagagtatcatagtgcaggagcggagcctgggcctggccgagaaggtagctgccatcaatt
actcggttttgagcttgggaaaatattacgctcgcaaaatctacaagactccatacgttcccatagacaaggag
gtgaagatagatgggttctacatgcgcatgacgctcaaggtcttgaccctgagcgatgatcttggggtgtatcg
caatgacagaatgcatcgcgcggttagcgccagcaggaggcgcgagttaagcgacagggaactgatgcacagtt
tgcaaagagctctgactggagctggaaccgagggtgagaattacttcgacatgggagctgacttgcagtggcag
cctaatcgcagggctctgagcgccgcgacggcaggatgtgagcttccttacatagaagaggcggatgaaggcga
ggaggaagagggcgagtacttggaagactgatggcacaacccgtgttttttgctagatggaacagcaagcaccg
gatcccgcaatgcgggcggcgctgcagagccagccgtccggcattaactcctcggacgattggacccaggccat
gcaacgtatcatggcgttgacgactcgcaaccccgaagcctttagacagcaaccccaggccaaccgtctatcgg
ccatcatggaagctgtagtgccttcccgatctaatcccactcatgagaaggtcctggccatcgtgaacgcgttg
gtggagaacaaagctattcgtccagatgaggccggactggtatacaacgctctcttagaacgcgtggctcgcta
caacagtagcaatgtgcaaaccaatttggaccgtatgataacagatgtacgcgaagccgtgtctcagcgcgaaa
ggttccagcgtgatgccaacctgggttcgctggtggcgttaaatgctttcttgagtactcagectgetaatgtg
ccgcgtggtcaacaggattatactaactttttaagtgctttgagactgatggtatcagaagtacctcagagcga
agtgtatcagtccggtcctgattacttctttcagactagcagacagggcttgcagacggtaaatctgagccaag
cttttaaaaaccttaaaggtttgtggggagtgcatgccccggtaggagaaagagcaaccgtgtctagcttgtta
acteegaacteeegectgttattactgttggtagetectttcaccgacageggtagcatcgaccgtaattecta
tttgggttacctactaaacctgtatcgcgaagccatagggcaaagtcaggtggacgagcagacctatcaagaaa
ttacccaagtcagtcgcgctttgggacaggaagacactggcagtttggaagccactctgaacttcttgcttacc
aatcggtctcaaaagatccctcctcaatatgctcttactgcggaggaggagaggatccttagatatgtgcagca
gagcgtgggattgtttctgatgcaagagggggcaactccgactgcagcactggacatgacagcgcgaaatatgg
agcccagcatgtatgccagtaaccgacctttcattaacaaactgctggactacttgcacagagctgccgctatg
aactctgattatttcaccaatgccatcttaaacccgcactggctgcccccacctggtttctacacgggcgaata
tgacatgcccgaccctaatgacggatttctgtgggacgacgtggacagcgatgttttttcacctctttctgatc
atcgcacgtggaaaaaggaaggcggtgatagaatgcattcttctgcatcgctgtccggggtcatgggtgctacc
gcggctgagcccgagtctgcaagtccttttcctagtctacccttttctctacacagtgtacgtagcagcgaagt
gggtagaataagtcgcccgagtttaatgggcgaagaggagtacctaaacgattccttgctcagaccggcaagag
aaaaaaatttcccaaacaatggaatagaaagtttggtggataaaatgagtagatggaagacttatgctcaggat
cacagagacgagcctgggatcatggggactacaagtagagcgagccgtagacgccagcgccatgacagacagag
gggtcttgtgtgggacgatgaggattcggccgatgatagcagcgtgttggacttgggtgggagaggaaggggca
acccgtttgctcatttgcgccctcgcttgggtggtatgttgtgaaaaaaaataaaaaagaaaaactcaccaagg
ccatggcgacgagcgtacgttcgttcttctttattatctgtgtctagtataatgaggcgagtcgtgctaggcgg
agcggtggtgtatccggagggtcctcctccttcgtacgagagcgtgatgcagcagcagcaggcgacggcggtga
tgcaatccccactggaggctccctttgtgcctccgcgatacctggcacctacggagggcagaaacagcattcgt
tactcggaactggcacctcagtacgataccaccaggttgtatctggtggacaacaagtcggcggacattgcttc
tctgaactatcagaatgaccacagcaacttcttgaccacggtggtgcagaacaatgactttacccctacggaag
ccagcacccagaccattaactttgatgaacgatcgcggtggggcggtcagctaaagaccatcatgcatactaac
atgccaaacgtgaacgagtatatgtttagtaacaagttcaaagcgcgtgtgatggtgtccagaaaacctcccga
cggtgctgcagttggggatacttatgatcacaagcaggatattttggaatatgagtggttcgagtttactttgc
cagaaggcaacttttcagttactatgactattgatttgatgaacaatgccatcatagataattacttgaaagtg
ggtagacagaatggagtgcttgaaagtgacattggtgttaagttcgacaccaggaacttcaagctgggatggga
tcccgaaaccaagttgatcatgcctggagtgtatacgtatgaagccttccatcctgacattgtcttactgcctg
gctgcggagtggattttaccgagagtcgtttgagcaaccttcttggtatcagaaaaaaacagccatttcaagag
ggttttaagattttgtatgaagatttagaaggtggtaatattccggccctcttggatgtagatgcctatgagaa
cagtaagaaagaacaaaaagccaaaatagaagctgctacagctgctgcagaagctaaggcaaacatagttgcca
gcgactctacaagggttgctaacgctggagaggtcagaggagacaattttgcgccaacacctgttccgactgca
gaatcattattggccgatgtgtctgaaggaacggacgtgaaactcactattcaacctgtagaaaaagatagtaa
gaatagaagctataatgtgttggaagacaaaatcaacacagcctatcgcagttggtatctttcgtacaattatg
gcgatcccgaaaaaggagtgcgttcctggacattgctcaccacctcagatgtcacctgcggagcagagcaggtt
tactggtcgcttccagacatgatgaaggatcctgtcactttccgctccactagacaagtcagtaactaccctgt
ggtgggtgcagagcttatgcccgtcttctcaaagagcttctacaacgaacaagctgtgtactcccagcagctcc
gccagtccacctcgcttacgcacgtcttcaaccgctttcctgagaaccagattttaatccgtccgccggcgccc
accattaccaccgtcagtgaaaacgttcctgctctcacagatcacgggaccctgccgttgcgcagcagtatccg
gggagtccaacgtgtgaccgttactgacgccagacgccgcacctgtccctacgtgtacaaggcactgggcatag
tcgcaccgcgcgtcctttcaagccgcactttctaaaaaaaaaatgtccattcttatctcgcccagtaataacac
cggttggggtctgcgcgctccaagcaagatgtacggaggcgcacgcaaacgttctacccaacatcccgtgcgtg
ttcgcggacattttcgcgctccatggggtgccctcaagggccgcactcgcgttcgaaccaccgtcgatgatgta
atcgatcaggtggttgccgacgcccgtaattatactcctactgcgcctacatctactgtggatgcagttattga
cagtgtagtggctgacgctcgcaactatgctcgacgtaagagccggcgaaggcgcattgccagacgccaccgag
ctaccactgccatgcgagccgcaagagctctgctacgaagagctagacgcgtggggcgaagagccatgcttagg
gcggccagacgtgcagcttcgggcgccagcgccggcaggtcccgcaggcaagcagccgctgtcgcagcggcgac
tattgccgacatggcccaatcgcgaagaggcaatgtatactgggtgcgtgacgctgccaccggtcaacgtgtac
ccgtgcgcacccgtccccctcgcacttagaagatactgagcagtctccgatgttgtgtcccagcggcgaggatg
tccaagcgcaaatacaaggaagaaatgctgcaggttatcgcacctgaagtctacggccaaccgttgaaggatga
aaaaaaaccccgcaaaatcaagcgggttaaaaaggacaaaaaagaagaggaagatggcgatgatgggctggcgg
agtttgtgcgcgagtttgccccacggcgacgcgtgcaatggcgtgggcgcaaagttcgacatgtgttgagacct
ggaacttcggtggtctttacacccggcgagcgttcaagcgctacttttaagcgttcctatgatgaggtgtacgg
ggatgatgatattcttgagcaggcggctgaccgattaggcgagtttgcttatggcaagcgtagtagaataactt
ccaaggatgagacagtgtcaatacccttggatcatggaaatcccacccctagtcttaaaccggtcactttgcag
caagtgttacccgtaactccgcgaacaggtgttaaacgcgaaggtgaagatttgtatcccactatgcaactgat
ggtacccaaacgccagaagttggaggacgttttggagaaagtaaaagtggatccagatattcaacctgaggtta
aagtgagacccattaagcaggtagcgcctggtctgggggtacaaactgtagacattaagattcccactgaaagt
atggaagtgcaaactgaacccgcaaagcctactgccacctccactgaagtgcaaacggatccatggatgcccat
gcctattacaactgacgccgccggtcccactcgaagatcccgacgaaagtacggtccagcaagtctgttgatgc
ccaattatgttgtacacccatctattattcctactcctggttaccgaggcactcgctactatcgcagccgaaac
agtacctcccgccgtcgccgcaagacacctgcaaatcgcagtcgtcgccgtagacgcacaagcaaaccgactcc
cggcgccctggtgcggcaagtgtaccgcaatggtagtgcggaacctttgacactgccgcgtgcgcgttaccatc
cgagtatcatcacttaatcaatgttgccgctgcctccttgcagatatggccctcacttgtcgccttcgcgttcc
catcactggttaccgaggaagaaactcgcgccgtagaagagggatgttgggacgcggaatgcgacgctacaggc
gacggcgtgctatccgcaagcaattgcggggtggttttttaccagccttaattccaattatcgctgctgcaatt
ggcgcgataccaggcatagcttccgtggcggttcaggcctcgcaacgacattgacattggaaaaaaaacgtata
aataaaaaaaaatacaatggactctgacactcctggtcctgtgactatgttttcttagagatggaagacatcaa
tttttcatccttggctccgcgacacggcacgaagccgtacatgggcacctggagcgacatcggcacgagccaac
tgaacgggggcgccttcaattggagcagtatctggagcgggcttaaaaattttggctcaaccataaaaacatac
gggaacaaagcttggaacagcagtacaggacaggcgcttagaaataaacttaaagaccagaacttccaacaaaa
agtagtcgatgggatagcttccggcatcaatggagtggtagatttggctaaccaggctgtgcagaaaaagataa
acagtcgtttggacccgccgccageaaccccaggtgaaatgcaagtggaggaagaaattcctccgccagaaaaa
cgaggcgacaagcgtccgcgtcccgatttggaagagacgctggtgacgcgcgtagatgaaccgccttcttatga
ggaagcaacgaagcttggaatgcccaccactagaccgatagccccaatggccaccggggtgatgaaaccttctc
agttgcatcgacccgtcaccttggatttgccccctccccctgctgctactgctgtacccgcttctaagcctgtc
gctgccccgaaaccagtcgccgtagccaggtcacgtcccgggggcgctcctcgtccaaatgcgcactggcaaaa
tactctgaacagcatcgtgggtctaggcgtgcaaagtgtaaaacgccgtcgctgcttttaattaaatatggagt
agcgcttaacttgcctatctgtgtatatgtgtcattacacgccgtcacagcagcagaggaaaaaaggaagaggt
cgtgcgtcgacgctgagttactttcaagatggccaccccatcgatgctgccccaatgggcatacatgcacatcg
ccggacaggatgcttcggagtacctgagtccgggtctggtgcagttcgcccgcgccacagacacctacttcaat
ctgggaaataagtttagaaatcccaccgtagcgccgacccacgatgtgaccaccgaccgtagccagcggctcat
gttgcgcttcgtgcccgttgaccgggaggacaatacatactcttacaaagtgcggtacaccctggccgtgggcg
acaacagagtgctggatatggccagcacgttctttgacattaggggcgtgttggacagaggtcccagtttcaaa
ccctattctggtacggcttacaactctctggctcctaaaggcgctccaaatgcatctcaatggattgcaaaagg
cgtaccaactgcagcagccgcaggcaatggtgaagaagaacatgaaacagaggagaaaactgctacttacactt
ttgccaatgctcctgtaaaagccgaggctcaaattacaaaagagggcttaccaataggtttggagatttcagct
gaaaacgaatctaaacccatctatgcagataaactttatcagccagaacctcaagtgggagatgaaacttggac
tgacctagacggaaaaaccgaagagtatggaggcagggctctaaagcctactactaacatgaaaccctgttacg
ggtcctatgcgaagcctactaatttaaaaggtggtcaggcaaaaccgaaaaactcggaaccgtcgagtgaaaaa
attgaatatgatattgacatggaattttttgataactcatcgcaaagaacaaacttcagtcctaaaattgtcat
gtatgcagaaaatgtaggtttggaaacgccagacactcatgtagtgtacaaacctggaacagaagacacaagtt
ccgaagctaatttgggacaacagtctatgcccaacagacccaactacattggcttcagagataactttattgga
ctcatgtactataacagtactggtaacatgggggtgctggctggtcaagcgtctcagttaaatgcagtggttga
cttgcaggacagaaacacagaactttcttaccaactcttgcttgactctctgggcgacagaaccagatacttta
gcatgtggaatcaggctgtggacagttatgatcctgatgtacgtgttattgaaaatcatggtgtggaagatgaa
cttcccaactattgttttccactggacggcataggtgttccaacaaccagttacaaatcaatagttccaaatgg
agaagataataataattggaaagaacctgaagtaaatggaacaagtgagatcggacagggtaatttgtttgcca
tggaaattaaccttcaagccaatctatggcgaagtttcctttattccaatgtggctctgtatctcccagactcg
tacaaatacaccccgtccaatgtcactcttccagaaaacaaaaacacctacgactacatgaacgggcgggtggt
gccgccatctctagtagacacctatgtgaacattggtgccaggtggtctctggatgccatggacaatgtcaacc
cattcaaccaccaccgtaacgctggcttgcgttaccgatctatgcttctgggtaacggacgttatgtgcctttc
cacatacaagtgcctcaaaaattcttcgctgttaaaaacctgctgcttctcccaggctcctacacttatgagtg
gaactttaggaaggatgtgaacatggttctacagagttccctcggtaacgacctgcgggtagatggcgccagca
tcagtttcacgagcatcaacctctatgctacttttttccccatggctcacaacaccgcttccacccttgaagcc
atgctgcggaatgacaccaatgatcagtcattcaacgactacctatctgcagctaacatgctctaccccattcc
tgccaatgcaaccaatattcccatttccattccttctcgcaactgggcggctttcagaggctggtcatttacca
gactgaaaaccaaagaaactccctctttggggtctggatttgacccctactttgtctattctggttctattccc
tacctggatggtaccttctacctgaaccacacttttaagaaggtttccatcatgtttgactcttcagtgagctg
gcctggaaatgacaggttactatctcctaacgaatttgaaataaagcgcactgtggatggcgaaggctacaacg
tagcccaatgcaacatgaccaaagactggttcttggtacagatgctcgccaactacaacatcggctatcagggc
ttctacattccagaaggatacaaagatcgcatgtattcatttttcagaaacttccagcccatgagcaggcaggt
ggttgatgaggtcaattacaaagacttcaaggccgtcgccataccctaccaacacaacaactctggctttgtgg
gttacatggctccgaccatgcgccaaggtcaaccctatcccgctaactatccctatccactcattggaacaact
gccgtaaatagtgttacgcagaaaaagttcttgtgtgacagaaccatgtggcgcataccgttctcgagcaactt
catgtctatgggggcccttacagacttgggacagaatatgctctatgccaactcagctcatgctctggacatga
cctttgaggtggatcccatggatgagcccaccctgctttatcttctcttcgaagttttcgacgtggtcagagtg
catcagccacaccgcggcatcatcgaggcagtctacctgcgtacaccgttctcggccggtaacgctaccacgta
agaagcttcttgettcttgcaaatagcagctgcaaccatggcctgcggatcccaaaacggctccagegageaag
agctcagagccattgtccaagacctgggttgcggaccctattttttgggaacctacgataagcgcttcccgggg
ttcatggcccccgataagctcgcctgtgccattgtaaatacggccggacgtgagacggggggagagcactggtt
ggctttcggttggaacccacgttctaacacctgctacctttttgatccttttggattctcggatgatcgtctca
aacagatttaccagtttgaatatgagggtctcctgcgccgcagcgctcttgctaccaaggaccgctgtattacg
ctggaaaaatctacccagaccgtgcagggcccccgttctgccgcctgcggacttttctgctgcatgttccttca
cgcctttgtgcactggcctgaccgtcccatggacggaaaccccaccatgaaattgctaactggagtgccaaaca
acatgcttcattctcctaaagtccagcccaccctgtgtgacaatcaaaaagcactctaccattttcttaatacc
cattcgccttattttcgctctcatcgtacacacatcgaaagggccactgcgttcgaccgtatggatgttcaata
atgactcatgtaaacaacgtgttcaataaacatcactttatttttttacatgtatcaaggctctggattactta
tttatttacaagtcgaatgggttctgacgagaatcagaatgacccgcaggcagtgatacgttgcggaactgata
cttgggttgccacttgaattcgggaatcaccaacttgggaaccggtatatcgggcaggatgtcactccacagct
ttctggtcagctgcaaagctccaagcaggtcaggagccgaaatcttgaaatcacaattaggaccagtgctctga
gcgcgagagttgcggtacaccggattgcagcactgaaacaccatcagegacggatgtctcacgcttgccagcac
ggtgggatctgcaatcatgcccacatccagatcttcagcattggcaatgctgaacggggtcatcttgcaggtct
gcctacccatggcgggcacccaattaggcttgtggttgcaatcgcagtgcagggggatcagtatcatcttggcc
tgatcctgtctgattcctggatacacggctctcatgaaagcatcatattgcttgaaagcctgctgggctttact
accctcgggataaaacatcccgcaggacctgctcgaaaactggttagcctgcacagccggcatcattcacacag
cagcgggcgtcattgttggctatttgcaccacacttctgccccagcggttttgggtgattttggttegeteggg
attctcctttaaggctcgttgtccgttctcgctggccacatccatctcgataatctgctccttctgaatcataa
tattgccatgcaggcacttcagcttgccctcataatcattgcagccatgaggccacaacgcacagcctgtacat
tcccaattatggtgggcgatctgagaaaaagaatgtatcattccctgcagaaatcttcccatcatcgtgctcag
tgtcttgtgactagtgaaagttaactggatgcctcggtgctcttcgtttacgtactggtgacagatgcgcttgt
attgttegtgttgctcaggcattagtttaaaacaggttctaagttegttatecageetgtacttetccatcage
agacacatcacttccatgcctttctcccaagcagacaccaggggcaagctaatcggattcttaacagtgcaggc
agcagctcctttagccagagggtcatctttagcgatcttctcaatgcttcttttgccatccttctcaacgatgc
gcacgggcgggtagctgaaacccactgetacaagttgcgcctcttctctttcttcttegetgtcttgactgatg
tcttgcatggggatatgtttggtcttccttggcttctttttggggggtatcggaggaggaggactgtcgctccg
ttccggagacagggaggattgtgacgtttcgctcaccattaccaactgactgtcggtagaagaacctgacccca
cacggcgacaggtgtttttcttcgggggcagaggtggaggcgattgcgaagggctgcggtccgacctggaaggc
ggatgactggcagaaccccttccgcgttcgggggtgtgetccctgtggcggtegettaactgatttccttcgcg
getggecattgtgttctcctaggcagagaaacaacagacatggaaactcagecattgetgtcaacatcgccacg
agtgccatcacatctcgtcctcagcgacgaggaaaaggagcagagcttaagcattccaccgcccagtcctgcca
ccacctctaccctagaagataaggaggtcgacgcatctcatgacatgcagaataaaaaagcgaaagagtctgag
acagacatcgagcaagacccgggctatgtgacaccggtggaacacgaggaagagttgaaacgctttctagagag
agaggatgaaaactgcccaaaacagcgagcagataactatcaccaagatgctggaaatagggatcagaacaccg
actacctcatagggcttgacggggaagacgcgctccttaaacatctagcaagacagtegetcatagtcaaggat
gcattattggacagaactgaagtgcccatcagtgtggaagagctcagctgcgcctacgagcttaaccttttttc
acctegtactccccccaaacgtcagccaaacggcacctgcgagccaaatcctegettaaacttttatccagctt
ttgctgtgccagaagtactggctacctatcacatcttttttaaaaatcaaaaaattccagtctcctgccgcgct
aatcgcacccgcgccgatgccctactcaatctgggacctggttcacgcttacctgatatagcttccttggaaga
ggttccaaagatcttcgagggtctgggcaataatgagactcgggccgcaaatgctctgcaaaagggagaaaatg
gcatggatgagcatcacagcgttctggtggaattggaaggcgataatgccagactcgcagtactcaagcgaagc
gtcgaggtcacacacttcgcatatcccgctgtcaacctgccccctaaagtcatgacggcggtcatggaccagtt
actcattaagcgcgcaagtcccctttcagaagacatgcatgacccagatgcetgtgatgagggtaaaccagtgg
tcagtgatgagcagctaacccgatggctgggcaccgactctccccgggatttggaagagcgtcgcaagcttatg
atggccgtggtgctggttaccgtagaactagagtgtctccgacgtttctttaccgattcagaaaccttgcgcaa
actcgaagagaatctgcactacacttttagacacggctttgtgcggcaggcatgcaagatatctaacgtggaac
tcaccaacctggtttcctacatgggtattctgcatgagaatcgcctaggacaaagcgtgctgcacagcaccctt
aagggggaagceegeegtgattacatccgcgattgtgtctatctctacctgtgccacacgtggcaaaceggcat
gggtgtatggcagcaatgtttagaagaacagaacttgaaagagcttgacaagctcttacagaaatctcttaagg
ttctgtggacagggttcgacgagcgcaccgtcgcttccgacctggcagacctcatcttcccagagcgtctcagg
gttactttgcgaaacggattgcctgactttatgagccagagcatgcttaacaattttcgctctttcatcctgga
acgctccggtatcctgcccgccacctgctgcgcactgccctccgactttgtgcctctcacctaccgcgagtgcc
ccccgcegetatggagtcactgetacctgttccgtctggccaactatctctcctaccactcggatgtgategag
gatgtgageggagaeggettgetggagtgccactgccgctgcaatctgtgcacgccccaccggtccctagcttg
caacccccagttgatgagcgaaacccagataataggcacctttgaattgcaaggccccagcagccaaggcgatg
ggtcttctcctgggcaaagtttaaaactgaccccgggactgtggacctccgcctacttgcgcaagtttgctccg
gaagattaccacccctatgaaatcaagttctatgaggaccaatcacagcctccaaaggccgaactttcggcttg
cgtcatcacccagggggcaattctggcccaattgcaagccatccaaaaatcccgccaagaatttctactgaaaa
agggtaagggggtctaccttgacccccagaccggcgaggaactcaacacaaggttccctcaggatgtcccaacg
acgagaaaacaagaagttgaaggtgcagccgccgcccccagaagatatggaggaagattgggacagtcaggcag
aggaggcggaggaggacagtctggaggacagtctggaggaagacagtttggaggaggaaaacgaggaggcagag
gaggtggaagaagtaaccgccgacaaacagttatcctcggctgcggagacaagcaacagcgctaccatctccgc
tccgagtcgaggaacccggcggcgtcccagcagtagatgggacgagaccggacgcttcccgaacccaaccagcg
cttccaagaccggtaagaaggatcggcagggatacaagtcctggcgggggcataagaatgccatcatctcctgc
ttgcatgagtgcgggggcaacatatccttcacgcggcgctacttgctattccaccatggggtgaactttccgcg
caatgttttgcattactaccgtcacctccacagcccctactatagccagcaaatcccgacagtctcgacagata
aagacagcggcggcgacctccaacagaaaaccagcagcggcagttagaaaatacacaacaagtgcagcaacagg
aggattaaagattacagccaacgagccagcgcaaacccgagagttaagaaatcggatctttccaaccctgtatg
ccatcttccagcagagtcggggtcaagagcaggaactgaaaataaaaaaccgatctctgcgttcgctcaccaga
agttgtttgtatcacaagagcgaagatcaacttcagcgcactctcgaggacgccgaggctctcttcaacaagta
ctgcgcgctgactcttaaagagtaggcagcgaccgcgcttattcaaaaaaggcgggaattacatcatcctcgac
atgagtaaagaaattcccacgccttacatgtggagttatcaaccccaaatgggattggcagcaggcgcctccca
ggactactccacccgcatgaattggctcagcgccgggccttctatgatttctcgagttaatgatatacgcgcct
accgaaaccaaatacttttggaacagtcagctcttaccaccacgccccgccaacaccttaatcccagaaattgg
cccgccgccctagtgtaccaggaaagtcccgctcccaccactgtattacttcctcgagacgcccaggccgaagt
ccaaatgactaatgcaggtgcgcagttagctggcggctccaccctatgtcgtcacaggcctcggcataatataa
aacgcctgatgatcagaggccgaggtatecagetcaacgacgagtcggtgagctctccgcttggtctacgacca
gaeggaatettteagattgeeggetgcgggagatcttccttcacccctcgtcaggctgttctgactttggaaag
ttcgtcttcgcaaccccgctcgggcggaatcgggaccgttcaatttgtagaggagtttactccctctgtctact
teaaceectteteeggatetectgggcactacccggacgagttcataccgaacttcgacgcgattagegagtea
gtggacggctacgattgatgtctggtgacgcggctgagctatctcggctgcgacatctagaccactgccgccgc
tttegetgetttgcccgggaacttattgagttcatetacttcgaactccccaaggatcaccctcaaggtccggc
ccacggagtgcggattactatcgaaggcaaaatagactctcgcctgcaacgaattttctcccagcggcccgtgc
tgatcgagcgagaccagggaaacaccacggttagtaatcaattacggggtcattagttcatagcccatatatgg
agttgcgatcgctgcgggccatgtcatacaccgccttcagagcagccggacctatctgcccgttcgtgccgtcg
ttgttaatcaccacatggttattctgctcaaacgtcccggacgcctgcgaccggctgtctgccatgctgcccgg
tgtaccgacataaccgccggtggcatagecgcgcatcagecggtaaagattccccacgccaatccggctggttg
cctccttcgtgaagacaaactcaccacggtgaacaatcccegetggetcatatttgccgccggttcccgtaaat
cctccggttgcaaaatggaatttcgccgcagcggcctgaatggctgtaccgcctgacgcggatgcgccgccacc
aacagccccgccaatggcgctgccgatactcccgacaatccccaccattgcctgcttaagcagaatttctgtca
tcatggacagcacggaacgggtgaagctgcgccagttctgctcactgccggtcagcatcgccgccatattctgt
gcaataccatcaaaggtctgcgtggetgcactttttacctgcgacatactgtccgtggcgctctcttcccactc
actccagccggacttcaggcctgccatccagttcccgcgaagctggtcttcagccgcccaggtctttttctgct
ctgacatgacgttattcagcgccagcggattatcgccatactgttccttcaggcgctgttccgtggcttcccgt
tetgcctgeeggtcagtcageccccggcttttcgcatcaatggcggcccgttttgcccgttgetgetgtgegaa
tttatccgcctgctgcgccagcgcgttcaggcgctcctgatacgtaaccttgtcgccaagtgcagccagctggc
gtttgtactccagcgtctcatctttatgcgccagcagggatttctcctgtgcagacagctggcgacgttgcgcc
gcctcctccagtaccgcgaactgactctccgccttccacaaatcccggcgctgetggetgattttctcatttgc
tccggcatgcttctccagcgtccggagttctgcctgaagcgtcagcagggcagcatgagcactgtcttcctgac
gatcgcccgcagacaccttcacgctggactgtttcggctttttcagcgtcgcttcataatcctttttcgccgcc
gecatcagcgtgttgtaatccgcctgcaggattttcccgtctttcagtgccttgttcagttcttcctgacgggc
ggtatatttctccagcggcgtctgcagccgttcgtaagccttctgcgcctcttcggtatatttcagccgtgacg
cttcggtatcgctctgctgctgcgcatttttgtcctgttgagtctgctgctcagccttctttcgggcggcttca
agcgcaagacgggccttttcacgatcatcccagtaacgcgcccgcgcttcatcgttaacaaaataatcatcctt
gcgcagattccagatgtcgtctgctttcttatacgcagcctctgccttaatcagcatctcctgcgcggtatcag
gacgaccaatatccagcaccgcatcccacatggatttgaatgcccgcgcagtcctgtctgcccaggtctccagc
gtgcccatgttctctttcaggcggcgggtctggtcatcaaaccctttcgttgcggcctcgttcgccgcctgcaa
tgccccggcttcatcgccggaacgctgcaactgagcaacatacgcaatctgctccgccgacacgttatggaact
ggcgagccatcgccgtcagccccgacgtcgggtctgtggtcagcttcccgaaggcttcagcgaccttgtccacc
tccacgccggatgcagaggagaaacgcgccacactctggctgatggacgcaatctgagectcaccgcttacccc
cgccttaaccagtgcgctgagtgactcgctggtctggttaaacgtcagccctgccgcctgcccggctctggaca
ggaccagcatacgatctgccgtcagtcccgcctgattgccggaaaggaccagcgttttgttgaaatcggacagg
gttgagttgccctgataccaggcatacgccagcgcaccggtcgccaccgccagcgaggtggcccccaccatcgg
cagggtgatcgcaccggcaagccccctgaacatggggatcatcccgccgaaggagtccttcacctgccccccct
gttgcagcaggatcagccacggactttgcccgcctgcaagctgcgtggccacgtcggtgaactgtgcaggcagc
atacgcatggcggctttatactgcccgacggaaatccccgctttctgtgcagccagcgcctgtcggctcagcga
ctgttcaacgactgccgctgtttttttcgcatcactttccgtaccagaaaaatgacgcctgactctggccatct
gctcgtcaaatctggccgcatccagactcaaatcaacgacgtcgactaagctctagcatttgtgaaccatcacc
ctaatcaagttttttggggtcgaggtgccgtaaagcactaaatcggaaccctaaagggagcccccgatttagag
cttgacggggaaagccggcgaacgtggcgagaaaggaagggaagaaagcgaaaggagcgggcgctagggcgctg
gcaagtgtagcggtcacgctgcgcgtaaccaccacacccgccgcgcttaatgcgccgctacagggcgcgtgggg
ataccccctagagccccagctggttctttccgcctcagaagccatagagcccaccgcatccccagcatgcctgc
tattgtcttcccaatcctcccccttgctgtcctgccccaccccaccccccagaatagaatgacacctactcaga
caatgcgatgcaatttcctcattttattaggaaaggacagtgggagtggcaccttccagggtcaaggaaggcac
gggggaggggcaaacaacagatggctggcaactagaaggcacagtcgaggctgatcagcgggtttgctagctta
ggcgaaggcgatgggggtcttgaaggcgtgctggtactccacgatgcccagctcggtgttgctgtgcagctcct
ccacgcggcggaaggcgaacatggggcccccgttctgcaggatgctggggtggatggcgctcttgaagtgcatg
tggctgtccaccacgaagctgtagtagccgccgtcgcgcaggctgaaggtgcgggcgaagctgcccaccagcac
gttatcgcccatggggtgcaggtgctccacggtggcgttgctgcggatgatcttgtcggtgaagatcacgctgt
cctcggggaagccggtgcccaccaccttgaagtcgccgatcacgcggccggcctcgtagcggtagctgaagctc
acgtgcagcacgccgccgtcctcgtacttctcgatgcgggtgttggtgtagccgccgttgttgatggcgtgcag
gaaggggttctcgtagccgctggggtaggtgccgaagtggtagaagccgtagcccatcacgtggctcagcaggt
aggggctgaaggtcagggcgcctttggtgctcttcatcttgttggtcatgcggccctgctcgggggtgccctct
ccgccgcccaccagctcgaactccacgccgttcagggtgccggtgatgcggcactcgatcttcatggcgggcat
ggtggctagcctagccagcttgggtctccctatagtgagtcgtattaatttcgataagccagtaagcagtgggt
tctctagttagccagagagctctgcttatatagacctcccaccgtacacgcctaccgcccatttgcgtcaatgg
ggcggagttgttacgacattttggaaagtcccgttgattttggtgccaaaacaaactcccattgacgtcaatgg
ggtggagacttggaaatccccgtgagtcaaaccgctatccacgcccattgatgtactgccaaaaccgcatcacc
atggtaatagcgatgactaatacgtagatgtactgccaagtaggaaagtcccataaggtcatgtactgggcata
atgccaggcgggccatttaccgtcattgacgtcaatagggggcgtacttggcatatgatacacttgatgtactg
ccaagtgggcagtttaccgtaaatactccacccattgacgtcaatggaaagtccctattggcgttactatggga
acatacgtcattattgacgtcaatgggcgggggtcgttgggcggtcagccaggcgggccatttaccgtaagtta
tgtaacgcggaactccatatatgggctatgaactaatgaccccgtaattgattactattaataactacaataat
caatgtcaacgcgtatatctggcccgtacatcgcgaagcagcgcaaaacgcctaaccctaagcagattcttcat
gcaattaagcttcgcggtgcttcttcagtacgctacggcaaatgtcatcgacgtttttatccggaaactgctgt
ctggctttttttgatttcagaattagcctgacgggcaatgctgcgaagggcgttttcctgctgaggtgtcattg
aacaagtcccatgtcggcaagcataagcacacagaatatgaagcccgctgccagaaaaatgcattccgtggttg
tcatacctggtttctctcatctgcttctgctttcgccaccatcatttccagcttttgtgaaagggatgcggcta
acgtatgaaattcttcgtctgtttctactggtattggcacaaacctgattccaatttgagcaaggctatgtgcc
atctcgatactcgttcttaactcaacagaagatgctttgtgcatacagcccctcgtttattatttatctcctca
gccageegetgtgctttcagtggatttcggataacagaaaggccgggaaatacccagcctcgctttgtaacgga
gtagacgaaagtgattgcgcctacccggatattatcgtgaggatgcgtcatcgccattgctccccaaatacaaa
accaatttcagccagtgcctcgtccattttttcgatgaactccggcacgatctcgtcaaaactcgccatgtact
tttcatcccgctcaatcacgacataatgcaggccttcacgcttcatacgcgggtcatagttggcaaagtaccag
gcattttttcgcgtcacccacatgctgtactgcacctgggccatgtaagctgactttatggcctcgaaaccacc
gagecggaacttcatgaaatcccgggaggtaaacgggcatttcagttcaaggccgttgccgtcactgcataaac
catcgggagagcaggcggtacgcatactttcgtcgcgatagatgatcggggattcagtaacattcacgccggaa
gtgaattcaaacagggttctggcgtcgttctcgtactgttttccccaggccagtgctttagcgttaacttccgg
agccacaccggtgcaaacctcagcaagcagggtgtggaagtaggacattttcatgtcaggccacttctttccgg
agcggggttttgctatcacgttgtgaacttctgaagcggtgatgacgccgagccgtaatttgtgccacgcatca
tccccctgttcgacagctctcacatcgatcccggtacgctgcaggataatgtccggtgtcatgctgccaccttc
tgctctgcggctttctgtttcaggaatccaagagcttttactgcttcggcctgtgtcagttctgacgatgcacg
aatgtcgcggcgaaatatctgggaacagagcggcaataagtcgtcatcccatgttttatccagggcgatcagca
gagtgttaatctcctgcatggtttcatcgttaaccggagtgatgtcgcgttccggctgacgttctgcagtgtat
gcagtattttcgacaatgcgctcggcttcatccttgtcatagataccagcaaatccgaagcggccgcggaacaa
caacaattgcattcattttatgtttcaggttcagggggaggtgtggtcctgcgattccatcgagtgcacctaca
ccctgctgaagaccctatgcggcctaagagacctgctaccaatgaattaaaaaaaaatgattaataaaaaatca
cttacttgaaatcagcaataaggtctctgttgaaattttctcccagcagcacctcacttccctcttcccaactc
tggtattctaaaccccgttcagcggcatactttctccatactttaaaggggatgtcaaattttagctcctctcc
tgtacccacaatcttcatgtctttcttcccagatgaccaagagagtccggctcagtgactccttcaaccctgtc
tacccctatgaagatgaaagcacctcccaacaccccttttataacccagggtttatttccccaaatggcttcac
acaaagcccagacggagttcttactttaaaatgtttaaccccactaacaaccacaggcggatctetacagetaa
aagtgggagggggacttacagtggatgacactgatggtaccttacaagaaaacatacgtgctacagcacccatt
actaaaaataatcactctgtagaactatccattggaaatggattagaaactcaaaacaataaactatgtgccaa
attgggaaatgggttaaaatttaacaacggtgacatttgtataaaggatagtattaacaccttatggactggaa
taaaccctccacctaactgtcaaattgtggaaaacactaatacaaatgatggcaaacttactttagtattagta
aaaaatggagggcttgttaatggctacgtgtctctagttggtgtatcagacactgtgaaccaaatgttcacaca
aaagacagcaaacatccaattaagattatattttgactcttctggaaatctattaactgaggaatcagacttaa
aaattccacttaaaaataaatcttctacagcgaccagtgaaactgtagccagcagcaaagcctttatgccaagt
actacagcttatcccttcaacaccactactagggatagtgaaaactacattcatggaatatgttactacatgac
tagttatgatagaagtctatttcccttgaacatttctataatgctaaacagccgtatgatttcttccaatgttg
cctatgccatacaatttgaatggaatctaaatgcaagtgaatctccagaaagcaacatagctacgctgaccaca
tccccctttttcttttcttacattacagaagacgacaactaaaataaagtttaagtgtttttatttaaaatcac
aaaattcgagtagttattttgcctccaccttcccatttgacagaatacacagtcctttctccccggctggcctt
aaaaagcatcatatcatgggtaacagacatattcttaggtgttatattccacacggtttcctgtcgagccaaac
getcatcagtgatattaataaactccccgggcagctcacttaagttcatgtegetgteeagetgetgagccaca
ggctgctgtccaacttgcggttgcttaacgggcggcgaaggagaagtccacgcctacatgggggtagagtcata
atcgtgcatcaggatagggcggtggtgctgcagcagcgcgcgaataaactgctgccgccgccgctccgtcctgc
aggaatacaacatggcagtggtctcctcagcgatgattcgcaccgcccgcagcataaggcgccttgtcctccgg
gcacagcagcgcaccctgatctcacttaaatcagcacagtaactgcagcacagcaccacaatattgttcaaaat
cccacagtgcaaggcgctgtatccaaagctcatggcggggaccacagaacccacgtggccatcataccacaagc
gcaggtagattaagtggcgacccctcataaacacgctggacataaacattacctcttttggcatgttgtaattc
accacctcccggtaccatataaacctctgattaaacatggcgccatccaccaccatcctaaaccagctggccaa
aacctgcccgccggctatacactgcagggaaccgggactggaacaatgacagtggagagcccaggactcgtaac
catggatcatcatgctcgtcatgatgtcaatgttggcacaacacaggcacacgtgcatacacttcctcaggatt
acaagctcctcccgcgttagaaccatatcccagggaacaacccattcctgaatcagcgtaaatcccacactgca
gggaagacctcgcacgtaactcacgttgtgcattgtcaaagtgttacattcgggcagcagcggatgatcctcca
gtatggtagcgcgggtttctgtctcaaaaggaggtagacgatccctactgtacggagtgcgccgagacaaccga
gatcgtgttggtcgtagtgtcatgccaaatggaacgccggacgtagtcattctcgtattttgtatagcaaaacg
cggccctggcagaacacactcttcttcgccttctatcctgccgcttagcgtgttccgtgtgatagttcaagtac
agccacactcttaagttggtcaaaagaatgctggcttcagttgtaatcaaaactccatcgcatctaattgttct
gaggaaatcatccacggtagcatatgcaaatcccaaccaagcaatgcaactggattgcgtttcaagcaggagag
gagagggaagagacggaagaaccatgttaatttttattccaaacgatctcgcagtacttcaaattgtagatcgc
gcagatggcatctctcgcccccactgtgttggtgaaaaagcacagctaaatcaaaagaaatgcgattttcaagg
tgctcaacggtggcttccaacaaagcctccacgcgcacatccaagaacaaaagaataccaaaagaaggagcatt
ttctaactcctcaatcatcatattacattcctgcaccattcccagataattttcagctttccagccttgaatta
ttcgtgtcagttcttgtggtaaatccaatccacacattacaaacaggtcccggagggcgccctccaccaccatt
cttaaacacaccctcataatgacaaaatatcttgctcctgtgtcacctgtagcgaattgagaatggcaacatca
attgacatgcccttggctctaagttcttctttaagttctagttgtaaaaactctctcatattatcaccaaactg
cttagccagaagccccccgggaacaagagcaggggacgctacagtgcagtacaagcgcagacctccccaattgg
ctccagcaaaaacaagattggaataagcatattgggaaccaccagtaatatcatcgaagttgctggaaatataa
tcaggcagagtttcttgtagaaattgaataaaagaaaaatttgccaaaaaaacattcaaaacctctgggatgca
aatgcaataggttaccgcgctgcgctccaacattgttagttttgaattagtctgcaaaaataaaaaaaaaacaa
gcgtcatatcatagtagcctgacgaacaggtggataaatcagtctttccatcacaagacaagccacagggtctc
cagctcgaccctcgtaaaacctgtcatcgtgattaaacaacagcaccgaaagttcctcgcggtgaccagcatga
ataagtcttgatgaagcatacaatccagacatgttagcatcagttaaggagaaaaaacagccaacatagccttt
gggtataattatgcttaatcgtaagtatagcaaagccacccctcgcggatacaaagtaaaaggcacaggagaat
aaaaaatataattatttctctgctgctgtttaggcaacgtcgcccccggtccctctaaatacacatacaaagcc
tcatcagccatggcttaccagagaaagtacagcgggcacacaaaccacaagctctaaagtcactctccaacctc
tccacaatatatatacacaagccctaaactgacgtaatgggactaaagtgtaaaaaatcccgccaaacccaaca
cacaccccgaaactgcgtcaccagggaaaagtacagtttcacttccgcaatcccaacaagcgtcacttcctctt
tctcacggtacgtcacatcccattaacttacaacgtcattttcccacggccgcgccgccccttttaaccgttaa
ccccacagccaatcaccacacggcccacactttttaaaatcacctcatttacatattggcaccattccatctat
aaggtatattattgatgatgtt (SEQ ID NO: 180)
Mutated packaging signal sequence:
CATCATCAATAATATACCTTATAGATGGAATGGTGCCAATATGTAAATGAGGTGATTTTAAAAAGTGTGGGCCG
TGTGGTGATTGGCTGTGGGGTTAACGGTTAAAAGGGGCGGCGCGGCCGTGGGAAAATGACGTTATTTAAATATA
ACTTCGTATAGCATACATTATACGAAGTTATGAGGTAGTTTTGTTCAGGGGCAAGTGAAAATTGACCCATTACG
CGCGAAAACTGAATGAGGAAGTGTTTTTCTGAATAATGTGGTATTTATGGCAGGGTGGAGTATTTGACCGGATC
CAGGTAGACTTTGCTGATTTTCGTGGAGGTTTATAACTTCGTATAGCATACATTATACGAAGTTATATTTAAAT
(SEQ ID NO: 181)
Ad35 ITR 1-137bp:
CATCATCAATAATATACCTTATAGATGGAATGGTGCCAATATGTAAATGAGGTGATTTTAAAAAGTGTGGGCCG
TGTGGTGATTGGCTGTGGGGTTAACGGTTAAAAGGGGCGGCGCGGCCGTGGGAAAATGACGTT (SEQ ID
NO: 182)
XTEN linker: SGSETPGTSESATPES (SEQ ID NO: 183)
Loxp site: ATAACTTCGTATAGCATACATTATACGAAGTTAT (SEQ ID NO: 184)
Mutated packaging signal:
GAGGTAGTTTTGTTCAGGGGCAAGTGAAAATTGACCCATTACGCGCGAAAACTGAATGAGGAAGTGTTTTTCTG
AATAATGTGGTATTTATGGCAGGGTGGAGTATTTGACCGGATCCAGGTAGACTTTGCTGATTTTCGTGGAGGTT
T (SEQ ID NO: 185)
Vector from FIG. 29
Catcatcaataatataccttattttggattgaagccaatatgataatgagggggtggagtttgtgacgtggcgc
ggggcgtgggaacggggcgggtgacgtagtagtgtggcggaagtgtgatgttgcaagtgtggcggaacacatgt
aagcgacggatgtggcaaaagtgacgtttttggtgtgcgccggtgtacagccctaggataacttcgtatagcat
acattatacgaagttatactagtacgcccgggcgtatcgatacgatatcggtccggacaggaagtgacaatttt
cgcgcggttttaggcggatgttgtagtaaatttgggcgtaaccgagtaagatttggccattttcgcgggaaaac
tgaataagaggaagtgaaatctgaataattttgtgttactcatagcgcgtaatggatccgcgttaaccggcggc
cgcattctagacggaattcataacttcgtatagcatacattatacgaagttatgctagccgaagcttgagctcg
tcgagggatctgggcgtggttaagggtgggaaagaatatataaggtgggggtcttatgtagttttgtatctgtt
ttgcagca (SEQ ID NO: 186)
Adenovirus serotype 5
Catcatcaataatataccttattttggattgaagccaatatgataatgagggggtggagtttgtgacgtggcgc
ggggcgtgggaacggggcgggtgacgtagtagtgtggcggaagtgtgatgttgcaagtgtggcggaacacatgt
aagcgacggatgtggcaaaagtgacgtttttggtgtgcgccggtgtacacaggaagtgacaattttcgcgcggt
tttaggcggatgttgtagtaaatttgggcgtaaccgagtaagatttggccattttcgcgggaaaactgaataag
aggaagtgaaatctgaataattttgtgttactcatagcgcgtaatatttgtctagggccgcgggactttgaccg
tttacgtggagactcgcccaggtgtttttctcaggtgttttccgcgttccgggtcaaagttggcgttttattat
tatagtcagctgacgtgtagtgtatttatacccggtgagttcctcaagaggccactcttgagtgccagcgagta
gagttttctcctccgagccgctccgacaccgggactgaaaatgagacatattatctgccacggaggtgttatta
ccgaaga (SEQ ID NO: 187)
Adenovirus serotype
Catcatcaataatataccttatagatggaatggtgccaatatgtaaatgaggtgattttaaaaagtgtgggccg
tgtggtgattggctgtggggttaacggttaaaaggggcggcgcggccgtgggaaaatgacgttttatgggggtg
gagtttttttgcaagttgtcgcgggaaatgttacgcataaaaaggcttcttttctcacggaactacttagtttt
cccacggtatttaacaggaaatgaggtagttttgaccggatgcaagtgaaaattgctgattttcgcgcgaaaac
tgaatgaggaagtgtttttctgaataatgtggtatttatggcagggtggagtatttgttcagggccaggtagac
tttgacccattacgtggaggtttcgattaccgtgttttttacctgaatttccgcgtaccgtgtcaaagtcttct
gtttttacgtaggtgtcagctgatcgctagggtatttatacctcagggtttgtgtcaagaggccactcttgagt
gccagcgagaagagttttctcctctgcgccggcagtttaataataaaaaaatgagagatttgcgatttctgcct
caggaaat (SEQ ID NO: 188)
Exemplary HDAd35 donor vector (HDAd35-T4-Ef1a-mgmt)
(Ad35 5′end: 1→481; FRT (Complementary): 14126→14159; pT4 LIR:
14220→14463; EF1a: 14491→15825; mgmt: 15843→16466; pA: 16484→16705; pT4
RIR: 16735→17000; FRT (Complementary): 17107→17140; and Ad35 3′end:
28823→29230):
catcatcaataatataccttatagatggaatggtgccaatatgtaaatgaggtgattttaaaaagtgtgggccg
tgtggtgattggctgtggggttaacggttaaaaggggcggcgcggccgtgggaaaatgacgttttatgggggtg
gagtttttttgcaagttgtcgcgggaaatgttacgcataaaaaggcttcttttctcacggaactacttagtttt
cccacggtatttaacaggaaatgaggtagttttgaccggatgcaagtgaaaattgctgattttcgcgcgaaaac
tgaatgaggaagtgtttttctgaataatgtggtatttatggcagggtggagtatttgttcagggccaggtagac
tttgacccattacgtggaggtttcgattaccgtgttttttacctgaatttccgcgtaccgtgtcaaagtcttct
gtttttacgtaggtgtcagctgatcgctagggtatttaccggtattcaaggattacatgagcttagaaatgtaa
ttagcatagtgtgtggcatagtgtagataccaaataaatatgatctctccttctactcttgaaaatgcaaacac
attcttggtggtcctaaaatagcctgtaacatggtttactcagcagcatttgctattcaaggcagatctgcctt
tagtcattggctgcgctcctgaacagctgtgtgaaaggctaacttttgtaaaccaaatcaaaataaaatgcagc
aaaaatttgtcactgaaaggaaatcctcagtatatccttttatgaaatgaaagatccctcatccaaacttaact
tttttaaaagtgcgcatttggagatatagccctttcttatgaatcctaattcaattttggccataaacacacgt
tgatgttccccaccccaaagcacatagcaacaagagtaggttctatattgaaaataatgacaatttaaaaacat
gtacttatttcactgtatgtggacagtgtctatgattgcatcatgaagtgtcatataaccatgtacgtgtacat
gagagagagatagagagagaagtggtagggtggtggtggtagaggggatggcgatagtaatcatggtaatggta
gaggtgatggaggtggtaatgacggaggtaagggtggtagtgatgatggtggtggtggtaatggtggtggatgt
ggtggtggcaattgggatggtgggatggtggtagccatggtgatggtggtaatggtgttgatttaaagggtggt
ggtagtgaaggtgagggtagtggtggtggaggtggtggtgctggtagcaatagtgatggtggtgatggtgttga
tgagggtgttgggatcagggtgagttcccacagtatatttcattcttgttgtaccactctgtcaacagcaccac
tgactgggacagaggaagaaggcacactctgaatgtgttattaacagaaacctcaaaacagtctgtctccttgt
agtcattcaaaattatctttttcttacctggaaaactgaaactgaattaccgggaaaaacacaggagatttttg
tttgttaatatgctgccaataaagtaattttatgtcaaatttaactacaggaaagggcaaggcattttctaagt
tccttagatgtcatgtggctaaaaaaaacaaaaggatggacageagttagatactgtacacttagctgtttgaa
gccatatattcagaaagcagatgttgggagttggtgtttgaggactgatttcctggaggtattttatataggcc
aagttcattgttctaaactctaagggcttgacttgagggaggaaaagaggcaagaacatgtttagttttgctga
cagcatcacatgggcagccctaaggctagacaactttagggcctgaagcttattctaggaaagaagcacctaca
gagtggcactgggctcccctccactatagagatgaagtcatatgacagtaaagggcaggcagggctgcctaggg
ggcccagaactgacacttccattagaatgagcacaggccagggagagaagtggggaaccagagagaaggagctg
gaattctagtaggacaaacggtaagtgaacaacaagaacaagttaagagtgtgtgcagtattctttcaaagact
gaaaaaatagtgatgtgatagaatggcaggtggctctgagcaggccaggagaaggactgggggcagagcatccc
aggcaggagggcagcaagtgggaaggccctggggtggggcttttggactgttccagtgacgggcaggcagccag
tgtgcctgtcacacaatgcaccagggaagtagtcgtgaatttgcagagggtcttgcaggctatgggaaagggat
tggattgtattttgtttgtagggaagccatcgggggacttaagcagaggaaggattggcttcatctctttgaaa
aagttctctctggatgetgatgggaggagaaatggaaggaaaagaaacacttttaggggcaagaacttttgaga
agggtggaattgggagtgtggagttggggccagctttggcacaggaggggaagctaaacacgtggccgcatgag
ggcctgtaattctacctgaaatgggtaccatttgttagggtaaacaaatgaaccaaatgcccagtgatacagac
caagtgttggcaaacttcttctgtgatggcccaggtagtaaatgtctcaggcttcgcaggccatgtggtctctg
ttgaagctctgtgtagtagacaatatgttaatgactgggcgtgactgtgtgctaataaaagtttatttacaaaa
acageeegtgggetggatttageteacaggctgtagtttgccaacctctgacctagagcatgaactgagcatct
tcttggagggaaataagttctttccaagttgccctcctcacattgcagggggccatgtaggcccattattcaca
gaagagtgggtgggcaacctttctggagcagaaaaacgtaaagatttcttccgtagtgcaagtaaggtgaccat
ttctaaaccgtgcaagtgatccagcagtcccaaaagttgtttcacttctcattgtgcgcccgttctcaggtgct
ccgaagcttccagtcctttgtagggacatggatgaaattggaaatcatcattctcagtaaactatcgcaagaac
aaaaaaccaaacaccgcatattctcactcataggtgggaattgaacaatgagatcacatggacacgggaagggg
aacatcacattctggggactgttgtggggtggggggaggggggagggatagcattgggagatatacctaatgct
agatgacaagttagtgggtgcagcgcaccagtgtggcacatgtatacatatgtaactaacctgcacaatgtgca
catgtaccctaaaacttaaagtataataataaaagaaaaaaaaaaagagaggagagaaacatcatcccctccag
gatacccttgggccttgttcttatagtcttgtacattgttgaacaatttgcatgggetagtggattaaagcaca
ccctccaccctcaggccctcaagggtctctatgataatacagtctcaccttctaccctttccatcaccatccta
ggtgctatggccaaccttgaggctgccatgttaggtctatgcatttcccacctccaccacataactctctgaag
gccaggtagtttcctattcatcttggtaaccccaaagcctcgtgacagggctcagctggcatctgcggatgtga
atgaaccattggagaaaatggtactctgcaaataactctgttattttcccatttcctgtgtaaggcctagagac
aatgactttttaattgcaccccttcccctctgtatgacactggccttctcttgtgtccagcaatgtgggtggcc
tagatgatttctaagggacttctggccaagatgaacagcagctgcatcttactgagcatttactatgtgccata
tactcagecacagetctaggggcatagaagcaggagctctcagggtcagggcagtgagtgageaagcgageace
tatgccagccctgcctctggatggggacttgagagggtgatggaagcctgcagcactggagggaggcagacaaa
gacaggcctgtgetgagggggcceggageaagagagagggaggcaatgacagcagagacatgcetgcgccttgg
gtttgagtgcccagtggtcaaatccacttccctgtggctgatgcttgcctttctaactttggaatttaggggtt
ggagatctggtgagaaggtaggagggagatgaggaggagaagggaaaggcaggaaggaaggggagggaaaggaa
aagcaaaaggggaggaggaaggtttccaacaaattattctatatcaactgcggaaatcaaaatttgttgcccaa
atcttagaagctcatgtccctcctccccagaagtctggaatgcagcactccaggggtagcttataacccaaata
tctatctgtaaaaagagaaacattgggctttcgagctgtggattctcagtaaaagcaagaggcctcagcctaca
caggccagcccagagtttgaggaaccccaggcccacacccacagggctggcccctgggtctgcatactccctag
aaatgtgcacacttctgagcctcaactctgtcctggagtctaacagcatccctctccttcctggggcagttcca
cctccagaaacctgttaccttgggccttatgtcaaggaaactgtgggaaagagctaggcaggaatgcagatgag
gccagcatgggctcctaaaagtttagaaataggcagtgtcatgctcccaggtgcctgcataaaccagctgaaaa
atggagctcccctcaccagcactctcccttcaaacagactgtgatttgcaggtcactggtttaccaagccaggc
tacccaggcaggacccagatgccaagcccagtggtgtcctgcaagctgagcagtgctcagttcttgcaaaaaaa
ggtctgtgtgaaggcaaggcctctgcctggcttctcaccccagttgggtgtctggaacaggaaggagcccttac
tgcagaaaaaggaggagggagcaaagggagcgaacagctgcgtgctccatggggaggatccccaaagtagaaag
gcgcatacacactgcagcccttgacccagaatgctcacagctacattacagattcaggtctcctcagtgtagtg
gggctgctgatgagactgtggcatcctcaggggtcaggacacacattttccatcactcttctgatggcaaaaaa
cctctgagccaatgccaacctctgatcattaaaaaaaagtgctcacagcagtgtgtggtttaggatcatgccct
gtgtggtttggaacacgtgcacaaccacaccttgttcatcaccatcccagaaaccctgacgcaggcaaagagca
gagttattaaccctactttactgatgtggatactgaggcccagaggctcatgcaagttatcaataagtggcagg
gacagttgcctctagattaactagcccctaggatcacctgggtcttggaaggggacccataaacatgagctccc
ctctettggggccagatttgcacctgtgccgcgccttcagectgcatgaagtaggggctgetggcaaagactea
aagctgtaaatctgggttttctcttgaggcttctaagggagctgtttcgacaactcactctgttcccagctggc
tgcccctgcatagggttttaaagcagcctagctttctgccaggcttggcagtggacaacgctggtcagaacatc
ccagagagctaccagaatgaagtaagtttgcttctactctttacctgtttatgggctgtctctgccactggaat
gaaaggcactgagaacagtgcctggcctgcagaaggccctggaaatacctgagctcctaatctgggaataggag
taggaagagctttggaggcagggcacctgagtttgagatctacaacttcctgcctgtgtgacattgggaaagtc
tccatcctttctgagcctcagtctccaccctggggaagtggaaatatcaatctctgtgacacagaagcaaatga
gcgaatgtgcacaaagtaccttgcacaagagagacgctcaaacacttgcctccaggtttcaccgagaactacag
agtaagatagatttgttcccagtggaggaagcctgggaataatttgcccctagactatgaattcctggggctca
agatcgagcacagggccaggcacacagaagggaccctggaaatgtggcaggaggccagagatagacaggccctt
agagctcatacccatgccctctgacctcaagaagaaagaaacctgctcaaaatctcacaaagagcttgttccaa
ccctgaatcgagtctgaggactccttcctgagtccagcactttttctgcaagaagtatatgcctccaaagctga
tgggcgcaaatcttgaaccccgtcacataaacacaaagggaggaggtgactagagctcctcctactggatatgt
ctaaggtcaccagtctaaagaaaagggatggatagaatgaggccagtatttttgcagccatccaaatgtccaca
tacgctgttacactgagggctcctctctcccccgtcttcagccctacttgcatttagaggtgagaaagatatgg
gctgaggggttgtttttcatcgtattgtagatggaaagcacactgcccttggggccatccaaatgtggaccttg
atgtagcaccccaccttctggatggccatccttctgaaagtcactgaatttctcagactttattctctttatcc
ataaagaaggagaataataataatccccccaccctgcccaaccactgactggttgggaagctcagaagaaatac
tgggcacggcatcccattgtaatctatagagtgagtcgcttcttaatattaaatggctgaacacagaagatgtg
caaaaagtactgtgtccccttcctcctccaactgaacatttcatgccctttgcaccctcattttgtctaggagc
tgccttatgaagggaataggtacctgctccgagctggaggaatctttgccacttatggtggggtatggactgag
acagagatggcatgtgacatgcgcactgagtctcaactccatgcaggctctggagcactctcaaattggagtac
taatgccttttaaattctcacactagcaatcctttgacctactgatctagggatctagggaaagaatcgtgate
ttaacttcaaagggaaggacaaaatgttctgcctcctgttaaaactccatacactaagtgcagagactggatgc
cttattaaccttgggtagatgcccaaatgttcaaaaggtcaaactcttctgttccccagatcgccagagtcatt
aaccagtcacactattaaatgaatgaacagatgctgaaaaggtacttgcattactgagatttcttatggtgatg
gcccctgcctgatatgtattcagcattttgtagttttcaatgtgcattagagtatagtggtgatgacattggcc
tctgagtttgccacttcttatatctgtgactttggtcaaattgcttaatctctctgagtctcggtttcctggag
ataataatagcttcttcttcccagggttatcatgaggattacaggagataatgccccaaaaatgcttagtaaag
tgcctagcacctagtcaatgctgaattaaaggtggttattcttacttttcgttcatttgaactttgttctcagg
gagggcaaaggatagacaaagccccatagctagtgaggagtagctgcaagactagaacccaggtgttctgagcc
ctagtcttaggccaagaacaactgttacgtgagatgcacgttttccttcaagggagctcacaattatttccatg
taaattcaaggactgctaaaagagaactctcctctgggactgatatcattttatttcaagattgatttgaaaca
tgttttttgtttgtttgtttgttttctaggaaagaacaagagaaccagttaagctgaatgcctgaagcaaatcc
ctgttagcgatgttttcaggatgagggagagtggtgcaagaaacgtgcttccagatgcacatggtttcctggga
ctagggttcagggtgtcatccctgggtgttattaagtgtcagaaggagagcaaacaagggaaacatctgagatc
cagctaaggctacaccctggaaatgcaagcccagctcttgcaaaggacctcctttggccactcaccttccaggc
cttacaataacttgtttggactgcaggtttcttggtggactcacaggccattctgcttttatttggtcaacctc
agttcacaagcacccagatgctgagatcctcagcatgtgcagcagagtttcatattagcactgggtacctttct
gaggctacagggataccgtacagcagcacctgtcacgtccagccaaaggagtgggctctctcaatgtcatccaa
tgctgtttcaactgtgaagaagaccatctgagagagttgcttttggaggctgaggcaaatttttaaaattcttt
gttctcctcaactggggtgaattcttggtcttctaggacagcttgaagttttagaaagagtcaagccactcaga
accaacagagaactctttcagagaacaaggtgtggcatagaggaggcagagggctgatcttgatcaaatccaaa
gtgtgactctaaagcaatgaatgtgaatttttggcaaagcttacaaagggctctaaaggccatctgcaaagaga
agccaagcctgatcgatgaatcactagtgcggccggatatcgatcggcacgctgttgattttctcatagtaagg
aacagtgggccctttcagtcccacttctgtagtctgtggtactacaaatggtgagcccatgatgttgccattca
tagggttattctccagcagtaatgactggccagccactcccatagccgcggggctaggatttattgtcaatgga
gggacctgcagttctgcacaagcagtactaggatgagcacctgggcccattgcaagggtgacatcttcaaggca
aggcctcttaattttattagggtagcccccatcagecatgtctggaaactggaagtggtcttcttcttgtctcc
tcttaacagttccctgtgaatggaagagaagagaggaggagaagagaggagaggagaagggaagagaggtgaca
cacacacacacacacacacacacacacacagagagagagagagagagacagagagaaagagagagagagagaga
gaggaatttttataaaggtttggcacattaaagctaatgaacaggaaatgtgcatgataaaacagacctctcag
tttaaagacttatagttgtgaaaactataaaatacagcctgtctttggaaccatagtgcttatttattcattat
tatgtttcatctaaactgtctaattacatttcaaataaggcattatgttgtctgtatactaaaacgggatagaa
cgttattcaaagggtaatctgcccacttcaaggagagttcaacaaaactatgcagaagtcactaaatgaaccat
gctgccaaaggcaggcattggagagaaaactagaagtagctaaatagttttaattctttcctgtctacagacac
atagattttaacgaaggaataccatagtatagaattgaacttttaggctgccttctagtcttggttaaatgcat
caggctgcagtggtaaaattgaatacaacagagcccttacaggaaagaagtagatctggatgtgttttcttggg
gagctgtttaaaatactgtttttgggaaagcacaagtttcagaacagtcattgtaggcatcgtattcattgttc
catttatttttacacacacacacacacacacacacacacacactctcacacattgctatgtgtacacaaaaata
atttggaagaacctatacccaacaatttggagtggtcatttatttgggatgactggcaattccctttctattct
cttcatttctgcttgtttgtctttaacgagaacgactcataatccaaaaatttaaaaaagtataaagttatcta
aataagaaattttcctctgaagatgcatcctcaggttggggagatattaaacaatgagaaaaggccccaatctg
ggatctgaaccttgggggagctgcccatcatttatagaagcacagcctttgggaacaaagcaaagtcactagca
atgtgagacttcctactcttcatggcttcatacagtcatccatcgctgttgtgttaatgaccatgacctgtatg
ttagcaggtaaatgggaaaggaagtgggggcaaaggagtatgtgcaggaatgatcaaaataaggaaaggaagag
agggatctggaaatcacctgaatgccgataggtgaacaggtagaattcttttaaagcttcccccacccggtacc
ccccaaataacccctttccagctttggaagtttcactaggacatacagtgctcatcctctgatgtcaccttaag
tttggctcttctggtttgatgagcttgtagcccactaggagctcaaggcatgcatggggccacttgccagcacg
atgaggggcatgactgtcatggccaagtgaacatcaaagcagatccccagggctgtatgtctcaggccttggtg
cacatcagaatcacttagaaacatccacattcctgggccctcccaccacaaactgacagcttcatccagggtgt
ggcccaggcatcgggagtttttccaacagctccatggctgattctcaacagaaaaccactggcccagagcaagg
gtggaggcagcgtggcatagggctctgaccttggccttgccactgaacctctcagagccccagtttctttatgt
gtaaaatgagtgtaattatagttcttttctcatgaaggtgctctgactattaagtgaaacggggcacattgtat
gacacctaatagctcctcactaactggtacccggcattataaagggcaggtatggaagggttctgggagtccaa
tacccttcttaaagacagagaggtctctgagacccagagaggggcaggccttacccagagttgctcagccagag
ggcaacaaggcccaggtcagatgcagggcccctccaccaccactcagctgcctccagacccactgccttcgcca
tgttgttggtaggacactgcatcgcccccacagaaggggcttgccaacttgagtgagaggacttgcacacttct
ttgacttttcttttgagatgcccacaatctgaacaagggcacttcaagggacagctctgtcaccaaactcatct
gaggcctgaataccatgggtcaggcaggaatgggttggagaggtgtagagcaggcacaataagagggctgaggc
ccatgcagtcatcagtgcccactttcccaggagtctgactgggcacagcacccatagtgtccctgagctggtcc
atggagcagctcactaactgtttggcccacagcaggtgctcagtaaatggcagttgaacgaatcaatggacaaa
ggaacataaattacccaacacacagggagctcagccatttactcaatccattatggagtaacctacaaacaagc
cactgggtcccaaactgaaattgtgtctcttctacattctcccaaagaatccaataggttaaaaatagaaatgt
atgaaatagatcaatcagggatgattgcatgtggatttgacataaggatcccctgcagggagtctgagctggca
acagtcaggcccaaagtgctgtccatgatgtctcgaactgcaagacagttttaacaatggcgaagcaatgcaga
accaggcaggccaaggagggggtgggggttggggaaaggaagggagggaaggggctgtgaggggcaatggtctg
gcatccctgccacgtgagcctctgaaatttgctggcagcttctatgggctcccagagctttcacttaattgttg
gtctgccactaacctgctgggagtaaggtgcagggatggaggaggcagggcatgaccaccagacactaaaggta
ccagctggggccactggcaaagggaaggaggctgcacctctcctacatgagagcccgtatacacacaccttttc
cagcactcatcaactgcatcccaagcaaatggtccctgatcaattccaattctagaaaccaactgactactcaa
taacaaagtagatcccagcaggccgccactgctggagcggatgccacttttgctatgccaagtctgtggctgga
cagctgctggcatgtacactcactgactttcataaggatgcctaataaagggggcaggctcacctggcttttct
caggggtggggtttggggtgccgatagaggctgctgttttggcagagtggcaagctgcaagcctcttctgagct
ttcatttttcaatggacttcagtgagaattcactttgtcagaggccatgcagctccatgttttggatttcatgg
aatgagctttcaacagtgagectgaagtgccctggctgaacageaagaacaccagecaaccetaaacaaggceg
aggagaggcggctgtgtttacacggaaggctcagccttgctgtaatagcgtctgccttcaccagacatcagtga
ggcgtggaaatctattatccagttaattttgcccctagataaagacttgctttcgtgtcttctctttcacagtc
ccatgatctgttactcatctcaactgcgagaagttggctgggctttcccctgtgcccagtgccacactcgtgcc
ttcactgggtcacctgtgcctgtggctgatgccgctgaggttttgcctgcccagactgggtgtttctgactaaa
tcccacagccaccattttagatcaagggcaggagatagctcactgctccggaatgacctcccctcccagaatcc
tggtaggggcggaaggtccccaaccaagctcccagccctttctaaatgaatctccctgcttcacccatgtgctt
ttctccagtctctgcggtcttgatgacagcagggtattagtcctagctgtcccacagctcctacttctttcagg
cctctccctgtgacaatcagtagccactggcaggatttcctcagagcatatctcgatttgctttcagacaatta
gttaaaaggacactggaccccagacgtcccaactcccagccagagccctcacaggcccggcctttggtggtgag
gaagggggagggagtgagtgacagtgccctggcatcttttagaaacgaattcctttctctccatacataaatgc
ctgcagagtcccatttcagaatccggcagacaaagccaccaatgtgatccccatgaccttataaacattcatta
aaatgcatttcaaggcatgtgatggcctccccaccccctagataatgagaaaacaaaggtttctcttctgatag
agacaagttcagctctgaagtcaacattatttctggttctgtctgaacaatgacatatggcaactcttcccttt
ctatagttctagtccagaatgacaaaaaaggggaaaaatttcttagagaaggtagagattatacgaatacagtc
catgaaatgagcataaggagaataaagaatataacttatccaaagaagtctggcaggctgttataaatgcttga
ttttggacactgtagttggaggtttaacatggacaccaataaaaaggtcagcaaagggtatgcactgttcctat
tgggcaagaagataggaggtcaaaggtaaccaggaaagataaactcagggagacttattttccctccagagggc
actgggcttgtaggccctgggcaaaattgtcaaaaaggtgaaaatcgcctgtggtttatttagtctgctctttc
ttcactagtgcctcaccagttcagttcaggccaatttgctagaaggtagcgaacgatcgaccggtgaagttcct
atactttctagagaataggaacttcggaataggaacttctacctagatgcatgetcagagcggcccctagetag
cgtttaaaacctacagttgaagtcggaagtttacatacacttaagttggagtcattaaaactcgtttttcaact
actccacaaatttcttgttaacaaacaatagttttggcaagtcagttaggacatctactttgtgcatgacacaa
gtcatttttccaacaattgtttacagacagattatttcacttataattcactgtatcacaattccagtgggtca
gaagtgtacatacacgcgcttgactgtgcctttaagcttttaattaatggatcactagttgagtaattcataca
aaaggactcgcccctgccttggggaatcccagggaccgtcgttaaactcccactaacgtagaacccagagatcg
ctgcgttcccgccccctcaccegeeegetctcgtcatcactgaggtggagaagagcatgcgtgaggctccggtg
cccgtcagtgggcagagcgcacatcgcccacagtccccgagaagttggggggaggggtcggcaattgaaccggt
gcctagagaaggtggcgcggggtaaactgggaaagtgatgtcgtgtactggctccgcctttttcccgagggtgg
gggagaaccgtatataagtgcagtagtcgccgtgaacgttctttttcgcaacgggtttgccgccagaacacagg
taagtgccgtgtgtggttcccgcgggcctggcctctttacgggttatggcccttgcgtgccttgaattacttcc
acgcccctggctgcagtacgtgattcttgatcccgagcttcgggttggaagtgggtgggagagttcgaggcctt
gcgettaaggagccccttcgcctcgtgettgagttgaggcctggcctgggcgetggggccgccgcgtgcgaatc
tggtggcaccttcgcgcctgtctegetgctttcgataagtctctagccatttaaaatttttgatgacctgetgc
gacgctttttttctggcaagatagtcttgtaaatgcgggccaagatctgcacactggtatttcggtttttgggg
ccgcgggcggcgacggggcccgtgcgtcccagcgcacatgttcggcgaggcggggcctgcgagcgcggccaccg
agaatcggacgggggtagtctcaagctcgccggcctgctctggtgcctggcctcgcgccgccgtgtatcgcccc
gccctgggcggcaaggctggcccggtcggcaccagttgcgtgagcggaaagatggccgcttcccggccctgctg
cagggagctcaaaatggaggacgcggcgctcgggagagcgggcgggtgagtcacccacacaaaggaaaagggcc
tttccgtcctcageegtegettcatgtgactccacggagtaccgggcgccgtccaggcacctcgattagttetc
gagcttttggagtacgtcgtctttaggttggggggaggggttttatgcgatggagtttccccacactgagtggg
tggagactgaagttaggccagcttggcacttgatgtaattctccttggaatttgccctttttgagtttggatct
tggttcattctcaagcctcagacagtggttcaaagtttttttcttccatttcaggtgtcgtgagctagacggtc
gccaccatggacaaggattgtgaaatgaaacgcaccacactggacagccctttggggaagctggagctgtctgg
ttgtgagcagggtctgcacgaaataaagctcctgggcaaggggacgtctgcagctgatgccgtggaggtcccag
cccccgctgcggttctcggaggtccggagcccctgatgcagtgcacagcctggctgaatgcctatttccaccag
cccgaggctatcgaagagttccccgtgccagcgcttcaccatcccgttttccagcaagagtcgttcacgcgtca
ggtgttatggaagctgcttaaggttgtgaaattcggagaagtgatttcttaccagcaattggccgccctggccg
gcaaccccaaagccgcgcgagcagtgggaggcgccatgagaggcaatcctgtcaagatcctcatcccgtgccac
agagtggtctgcagcagcggagccgtgggcaactactccggagggctagccgtgaaggaatggcttctggccca
tgaaggccaccggttggggaagccaggcttgggagggagctcaggtctggcaggggcctggctcaagggagcgg
gagetacctcgggctccccgcctgetggccgaaactgaagcggcegetcttcgagcagacatgataagatacat
tgatgagtttggacaaaccacaactagaatgcagtgaaaaaaatgctttatttgtgaaatttgtgatgctattg
ctttatttgtaaccattataagctgcaataaacaagttaacaacaacaattgcattcattttatgtttcaggtt
cagggggaggtgtgggaggttttttaaagcaagtaaaacctctacaaatgtggtaaaactagtaatcgatttaa
ttaaagatctttaaacaatttaaaggcaatgctaccaaatactaagcgcgtgtatgtacacttctgacccactg
ggaatgtgatgaaagaaataaaagctgaaatgaatcattctctctactattattctgatatttcacattcttaa
aataaagtggtgatcctaactgaccttaagacagggaatctttactcggattaaatgtcaggaattgtgaaaaa
gtgagtttaaatgtatttggctaaggtgtatgtaaacttccgacttcaactgtagtttaaaacgggcccgtagt
ctagggccgccagtgtgatggagttcggcttcaggtacagcacactggcggccgttactaggtagctagagcct
tcagactctagggaagttcctatactttctagagaataggaacttcggaataggaacttcacccatggcgatcg
ctagcctctaactcctagaccgtcagaactgctgggcccttcaagacgggctgctcacacccactcatgttaag
cctggtgaggcctgtactctgttttcacaggaagaaatcctcacccagtcttccccaaacacattcccaggttg
tgtcattagtgggatagagatgattattgtggggagaagagaaacatctggatggatttggtgaggttgatcta
tagaggaagtaggtgctgcctgaggtagctgtaatagaagctaaaggtcaaaggagagggccctgtcccaatcc
agatgactccacttctgctggacccaggttcacaagcttaatctacatttcacctaaatttggctaacaagccc
aaaatcacacaggcaaagggagaagtggaggcagaaccgaggttggaggccaccagggccaccgggcagagatc
atttaagcccaaccttctcacttctccctgggctctgcctctcttaaaggaccttgtggtgtgacctcttgtag
gtccctttcacactcggggcctcagtttccccactgtaaagtgaatgggtcccagctttggtaagcttatgctt
acctgatgctttcttcctgggctgctcttgtagagaaaagataaatcttcttcctccatccacgagggctcctt
tccctgggggtgagagtaggctgaggagagccacttgcacacacccttaaagaaagtattacctgcaccagctc
agtgagaggcacagatcagactgttacttgaatcaaattatgagcctccccaaatatatctatgacatttaaat
aggggattacttgaacatagactgtgggatccggtgtggagtgcgggagactagcaaagtgaatcctgagagta
gcaggtctgcacctgttggatcgagaaaggcggcctacaattctggtcaaatgagctgtgcttattgacatatt
ctattagagagtactaccaggtcaccagtcaccagaaaggctgccagctctccaaccacctccagggaactatc
ctgaatggggccttaacaagtctaagagagggttggtttgggtcccaagccaatatttgctctgctttatgtca
gtcatatggaacccaaaccaaccctctcctatgtgcctcaccagtcggtgcagggatcccaatttcaagtttgg
ttttttatggtcaaagtccagcatagattaaatgaaggggtgtgatgatggtgttaaaagagaactccagacca
gtttaactcttggacacacatcccatctcaccatggtgcttccaaccttccagagatgatgggctcctattttc
tgatgacaaagccctccacaggattgctgcctggccatcagggagtgcctctgtaactgaggctgagatcccac
tttcagtcctccagctgtggcccatccctgctccgcccaccgggtatggcctgtcctaggctcttaggtatggc
tgcattgtgaaatgatggctacagagctggcatctcctgtagtctggttcatctagtgcactacctcatagtta
aaagaaatctgtttaagccactgagggtggctcctagtgccaactccaagaacaggaagcttcccttttttggg
aggaggggcagatggtaacatggatcgtccaggtcaatgggagcagggcaaccacagtaagtactggacaacaa
cacaaaactccatgtgtggcttccatcgagtccctctccaattggtttggtcttctccgtcccatgcagcactt
tagcaaggggcctggctgaaggctatgaattgtgtggagcctcctcattgcagtctccaaccatctgatgctgg
gaaaatgtcaccaggatgcagccatgccgtgtggccaatgaaccgagaaaacaccccttttctagaatgctcta
aagaggcagaataatccagaggtgaggaaggaaatactccaccagagacccaggcagttcctacaaaagccaga
ctttccttcacctagggagtgacaagaccagtggaaaacactctcaagcagtaacccccaaatgctctgcaagc
cagtggcgtccagataccgcacaagcgagtgggctgtctaatcccatcatcatgatgtaaatatctctaggctg
ccctgggctgtgcctgaccctgtcttcagctttccacacctccacctacagcccatgcacagaaggaccaccca
ggaatgctgcaagtgtggcacctccagggccacccagggagaaggagggcagctatgctggtggctccaggccc
atttggcgggtggtaccttcacaccacaaagcccaaactgaggccccagatttggctgatgagggcatattgga
caggggtcacttatgetcttccccattgccacctggcctctggctacctggaettggetacctgtggatoctet
cacaggtgccaccatcttggctgagtctccagatgcgaggtccctgaggcagtggcgggcttctegetaatget
gatgggattaggaatgggataggtggggagggccctggactgggccctgatgagccaagtgggtttttagaggg
gctactggtacatttcagggacaggacatctggtagagctaagctggggcaataaggagccactgctaatctga
gagctagaaacaatcagcttctgggtcattattaattagggtagtttgggctgtgtggaagtcacgtactatat
ggggtagccacagctctctctacagataatctctaagacttctgattgggactgtgtgaatgcagtagcaatat
ctcttcttactgccaggccctgccagtcctgcctccacgccctggctggccccccttatgatctgacccatgcc
aggctgccatagtatgttacttctgcattagcactccttgggacctgcctctccactgtccctcagactttaaa
gaactatacaaacccaaggggctcttcccaagagaattgatatgacttgaggtgattccatttctggaagtagt
cactccattttctgcctcactctttcagtgcttcacagagcaggttcgaacgaaggagccatccaactaaccgt
catgttcgggcaaccgaagaagggagtggcaggatttcctttggagacttctggaattagacagcagtttaatg
caagcatctaaattctctccctcccagagtctcattaaaactacagtaagagtttgtgttttgttttgttttta
aagacaaaatcccaccaggatagagagaataggagaggagataacagcatcataatttatgaaactaaaatgca
gatagaccaggattaactgactacacageaccaaggaagctgaatcacaagacagcagaggagaaaactggaaa
ggatcgtggtctatacggcagaatcttcccaagcctcaggaggaggagctctagatgttcccagatctgggagg
taaagtggaatggggggacatggtcagcgtaatggggttgggctggaagcaggttaaggagcaggcagatctct
gaatcccctctctgactctgtgtccccaggcatctgcctgtcccccaccctggaagaggtctggcttgaccctt
tgtctggtgaatttcctgetetgetttcctggtcctgetggccggatcagtggaggccactcacttcaceccac
agggatgttctgtgttgccctacacctgggaactggaggtactggaggcaggctgtggtgagcttgaaagcaaa
acacagagggcagtccaatctctttggccatatttcttctgcatatccaataccatgtccacaactctgctagt
gtcctgatggtggtgggctctacacattcccgggaagctgaaggcagataatgaccaggacaggtcaacctctc
ttcttctgaaagccttcatctactaatggcctgggactcttcccttaaatgcttagattgtgtcttccactaag
gttttttgctgttgctgttgtttgtttgtttgtttgtttgtttgtttgtttgttttgagacggaatctcactct
gtcgcccaggctggagtgtagtggcacaatctcagctcaccacaaccttcacctcctaggttgaaggggttctc
ctgcctcagcctcctgtgtagctaggattacaggcacatgccaccatgcctggctaatttttgtatttttggta
gagacaggatttcgccatgttggccaggctggtcttgaactcctgacctcaggtgatctgcctaccttggtctc
ccaaagtgctgggattacaggtgtgagccaccacacccggccaaggtttttgtttgtttgtttgtttgtttgtt
tgttttgtattgaggcagggtatcactctggtcacccaggctggagtgcagtagtgcaatcacggctcactgaa
acctccacctccctggcgggctcaggtgatcctgccacctcagcttcccaggtagctgggactacaggcttgta
ccaccactcccagctaatttttgcgtttttagtagagacagggtttccccatgttgcccaggttggtctcaaac
tctgggctcaagcgatctgcctgcctcagcctcccaaagtgctgggattacaggtgtaagccaccgtacccggc
cccgccactaaggttttgaaaatgaagcaattacaagtttaagtctattaataagtgatgaagccatgtagaaa
agcagaataattatcttggatcaggaaggtcacatgaggatctacttgggggttgtcaatattctatttcttga
cctgatcagtgttgacagcaggttttaattttttacttctttttgtttgtttgtttttgagacggagtcttgct
ctgtctcccaggctggagtgcagtggtatgatctcggctcactgcaacctccgcctcctgggttcaagctgttc
tcctgcctcagcctccccagtagctgggattacaggcaggcaccaccacgaccagctaatttttgtatttttag
tagagactgggtttcaccatcttggccaggctggtctcgaacttctgatctcgtgatccgccctccttggcctc
ccaaagtgctgggattacaggcttgagccagcgtgcccggcccattttttacttccttattaaactgtacatat
aggccttgcacacttttctgcatcaatgttatattccacaataaagggaaaaggtatatacacaacttgatacc
agtaatgtgaaacatatatttctacatagaaaaaaaaatgactgaaatactgcactccaatgtgttcacacagt
agttgtttctggattatttatatattaaatgtttatatattgtattatgccatgaggtttgtgttttctctcca
cttttctgcattttccaagtttactacaaagagcacatattactcttataatcagaaagtcataaaatatattt
aaaaagacaaaattgaaactaataaggatcaacacaaaacagatgagecatctgtggaaatccgcacagaatac
tacctaaagagattggtgacgtgcatgatctcactaggatgagcacaaagcttgccagagcctagggtctattt
ctagggttggctcttggaagccaggatagttgttatctctgggaagagggaggggcacacaaggggcttctaaa
acattctgaatgttctatttctgaacctggttggtgggtacatgactgttggttttattattatatgttttata
tactcttccgtatgtatggtgtggattccaaaaaaagatttcctttagagaaaaccagaatcacataagtagaa
aatatggtgctatgttgaaggaacaactcaagtttatataaaatcatcatcatttataggcttaaaaagttgct
ttggaattttggtctaactgacttgtcttttctgcagcaaaccacgctccttctggacgtgctccaggcagagg
ggattagggtgggttcaaggctgcaagtacctagctcagcacactctcttcaggggacttagagtttgtctggt
gttggctctctgagctcttgtcaggaatgccgacccttccgaggttcaggatttgaagcctgccttcccacccc
agatttggtccacacagacactcaagtatgtatttcaactacaaatgacctgtactttcctattactcctctct
ttcatggtaacctttctggtatccttccttccctacatttatgggagggggacatcattctctgctctcctgtc
actgaaggctccaccttctgtcttcttctgacccatctggttttcctggggccacctcctctccttaccaccct
aacgcttttgtaacttgaggagaaatgagagatcacctagtcaggtcatcattctctgtagatgaagaggccca
atggtttgctcaagaattgccaagcgagttaaagacagagagtatgagagtcagcaagacctacagaaagcatc
tatctgcactgttttgcagggacttagcctttgtgtgtggactcctggaatgccacccactaagaaacattgtc
tgacaccaactccccacttggtaggtggggacactgaaactcatggcaggaaagggccttgccccaagccaggg
cagagtgtcactcatcactctcaattttcagtccagggcaccttgttgtgactatcccaaaggcagccactttc
cctggtctgaaagacctgaagagagaagagaagagaaggatggaaggcagagtatgcggctttgattcatttcc
tggtgaaaacagatctatacgagaagcaaatttcacgaaagggaagagaagaaagtgtcccatacgttgctggc
ctgtttcaaccttgctttgattcttgctgaaaagggtaccgtgtatttctgagttcaacatgcagaccagtgtt
aggaaagccactgcacctccactttagcctccagggctgtgccctgcaaatggcctgcagccttggtgcctcgc
tctccagactgcattttggaagatgggacagaggcttatggaagcccacattagaacgggggagcagaatgggt
gagatgagggatccttgatagtgaaccagatgaaggaatggtagccaaatgccaggcctcctttgtggcttcaa
tccaaaggctctggagcccttccagggcagaacatcaggcatgtttacccccactgtcctcaacagtgacagag
gtgcaatcttgggcagctggccattttgaaagcaacctccttaatctcaactgggaaggctccctagcaggacc
cctgtgttgcacacctggaggaagctagactaaccagaagctcagcacggttccatctgggatgcccaggtctg
agacgaaaaaggtaactctcttttctgggtcctggcccagttgtgtctctctccacctcattctctgagatgcc
tgtctccccttttttgtcccatcaggaggcaagagctatcactgggccagactccaccagaagccaagccagct
tgttacccagcttctcagggagcaaagaacagccttgtttctatcttatccccactgtcccctgcccctgcccc
acctcccagecattcagcttctggcttccccagagctgcctgettctttgtggtcctccattccttgaaaagac
cttctagtcattagtgtatataaatggccacttagcccagattacagtgaggtcaacagctggggctctgagaa
ttgtcacacactggcacaggagaggaggctattcttccagagaatttggagggcactcccatccacttacaaca
aaaagcccatccactgtgcttggcagtaggtgatctgagaaccaatggaaccaggttaatcctgtggcactgtt
gagtgaggagagcagtggcgggcactggaaaatatcagagacaaggcaggagacctgaaatctaggcttagctc
ctcatatacttggcagctgtatgacctcagacaaccagtgttacctctctaagcctcagtttcctcatgcaaaa
ggagggggaataacaacagagcccactgcttgggggtgttgtgaggacaggatgaaaaaacaaacagaaatccc
tcagtacaggattcagtgcagtggacagtcttgcaaggtctggttcagccctccacccctaccctcaccagtat
aaagaactctggcctacaagtcagatgacctgagttttaatctcagctttgccattagecgtgtgaacttgaga
aagtccctttcctttttacatctattgggatgatcatgcattttttgtcctttattctgttaatatagtgtgtt
acattgattgcttttcatagactgaaccagccttgtattccagggataaatctcacttggtcatggtgtataat
cctttatacaaatgttgctgggttgagtttgctagtattttgttgaagatttttatgtcttgattcataaggaa
tattggtgtaccttccccttttatggccacagtttccctacaatgatgtagtcgaactagacaacctccaatat
ctttcagtattcatgtcctctgattctgtgaaactaagaaaattaagaaatagtgattcataggcacaaggcag
gcaaaacttagactccttgtagaataattaggaagccaaatattcagtgtgcttatttctcaaataaccttagt
ttctccagtctgccccaactccgaggcctgaatatctctagatgcttatgatggcaactaaagcctaaaagcta
attcattttaaagttcttccaaatgcatagggttttatttttccagacctgggttcagatggggaatttgacaa
acaatggaaagggggaaaaacaacaatctaaacactgagtgacaaagtaacaaagaaatagtctagetatcage
cagtcaagccagccttggctttgctatccaaagtagtcagtctaattctaccaccagtttctgttcctgtagct
gtctactgcctgccagggactctgccttcccacccacaactaccaatggaaggatgtggtgaccataccagtgg
ctgetgacatctcctgccatgggaagcataattgcctccagcagcctcccccttagatecatcatttttgttgc
acttggcctgggctgtactcccggccaatgactgaacatggtgagcatagtaatgcaggcccatttctgtgagg
agcaggactcctccagtaggtgactttggctcaaggactctctattggcctggttgaacttttcctgaactgtg
ctactgtctgagactcttcttacccaatcctctttctcgccccaattgtcacagaccacctgcattgtggtctg
agtctctccccaccttctcttgctcttccctgtttatctttcacaggcatttcccccagtacattccttgaatg
tctaacccgatacgggtgcctgacttttggcagacctaagcagacaaaaaggagtacttggttacctagctctt
ctttctaccacaaacatcgagggaaccctttttccctcacccctctgccacacccccactgccccagtgaacaa
ccacagagagagctgtggtataatattaggctggtgcaaaagtaattgcggtttttgccattacttttaatggt
aaaaaccgcaattacttttgcacctacctagtatttgtgtccccccaaattcatatgttgaaacctaacccaca
atatgatgtcattaggaggcaagaccttgaggaggtgattagatgatggggtggagctctcctgaatgagatta
gtgcccttataagaagaagcccaaggaagctaccttgactcttccatcacatgagaatgcagcaagaaggcacc
atctactaatcaggaagagagctctcaccagacactgaatctgccagtgtcttgatcttgaagttcccagcctc
cagaactatgcataatgcatttccattgtctctaagccacccagcctatggtattttgtcatagcagcctgaac
tgactaagacagtgagccacatgagaagtgccccaacccctcccttaagcacttggctcacagatcagtgggtt
catttctgcctgagttttattgttattctgtagatttcttgggctagatatatttttctgttattttccttctt
cacctcagtcatgaattggttgttttaaaaaagacaatgtaagtcatggggaaactcctgacaactctactctc
ctagggttcctgataaaaggggattcagttgagtcctctgatggtctctacctgccaaagtccagcagccctta
gcaaacatgctgctcgtttctgtagagaaggtgctggtgtcccaccatacttctctctccctcatgaagggctt
gcgacccagcaaatgggtggcttatatgggtctgtttcaaaggaagagccagctctgggaagaaaaacgatgag
cataagcataacctaccactgtgcctgggaaagcagacaacttttttgatgtgtgaatatctaatgagaatgga
atccatcaattaccttaaacttaggcacagtcttcaaattcaatatatgtgggatatacttttagtcagtttgt
agacgttatttgtaataaataatctggcttctctaaagaaattattttaagtgtttggtttggtttgatttaat
ggtaaaattatatttagtggcagagaattatagcaatggtgataaactatagagtgtcataagttcatatctta
ttctcacatttgaagctgcctgcagatgcattcaagatgcagccagaagtcaggagactcaggctgttatttgg
agctcatcattttacagccttgctggactcccactttctcaggggaaaaatgtggtgttgacccagattagctc
tccaggccctgctgagttgggcactctgtaagctggagggtcttctattgtcttcacctaagtgtcaatcaaca
acccaaatgggcatgggggaagagggagctgggccaatgcccagggtgcctggtagagagataccttgggcact
ggaaggcaccagcttcccagagagaagggggagggccatgaaaaagttggctgtagatgccagggacactggga
ctctccagctgtgtgtttgtgtcttctgaagacttatgtttcattcctttggagcatgcataatcatacactgt
gggatgtgttatatagattgcttgatagttcaccactgtaataaaatactgtgactggaatctgctcccagtct
gcctttgatagcacttgtgcaacacacatttactgagcatttacagtgatccaggacctgtgttgtgaaaacat
tgatggacaaggcagatggtggagcacgtcagtgaggatttttaacaaaggctggtaagtgctataaaggaaca
ttgtaggacactagagaacaaagaacaggagaacctgacttaggctggggtggggcgttggttagaggaggctc
cttggaggacatgaggtttaagctgtgacctgaggatgaatagatgttggccaggtgaggtaccggtatttgtc
agccttaccagtaaaaaagaaaacctattaaaaaaaaaatacacatacaaagcctcatcagecatggcttacca
gagaaagtacagcgggcacacaaaccacaagctctaaagtcactctccaacctctccacaatatatatacacaa
gccctaaactgacgtaatgggactaaagtgtaaaaaatcccgccaaacccaacacacaccccgaaactgcgtca
ccagggaaaagtacagtttcacttccgcaatcccaacaagcgtcacttcctctttctcacggtacgtcacatcc
cattaacttacaacgtcattttcccacggccgcgccgccccttttaaccgttaaccccacagccaatcaccaca
cggcccacactttttaaaatcacctcatttacatattggcaccattccatctataaggtatattattgatgatg
(SEQ ID NO: 189)
Exemplary HDAd35 donor vector (HDAd35-T4-Ef1a-mgmt-GFP)
(Ad35 5′end: 1→481; FRT (Complementary): 14126→14159; pT4 LIR:
14220→14463; Ef1a promoter: 14478→15812; mgmt: 15830→16450;2A:
16451→16522; GFP: 16523→17242; SV40pA: 17269→17390; pT4 RIR: 17501→17766;
FRT (Complementary): 17873→17906; and Ad35 3′end: 29589→29996):
catcatcaataatataccttatagatggaatggtgccaatatgtaaatgaggtgattttaaaaagtgtgggccg
tgtggtgattggctgtggggttaacggttaaaaggggcggcgcggccgtgggaaaatgacgttttatgggggtg
gagtttttttgcaagttgtcgcgggaaatgttacgcataaaaaggcttcttttctcacggaactacttagtttt
cccacggtatttaacaggaaatgaggtagttttgaccggatgcaagtgaaaattgctgattttcgcgcgaaaac
tgaatgaggaagtgtttttctgaataatgtggtatttatggcagggtggagtatttgttcagggccaggtagac
tttgacccattacgtggaggtttcgattaccgtgttttttacctgaatttccgcgtaccgtgtcaaagtcttct
gtttttacgtaggtgtcagctgatcgctagggtatttaccggtattcaaggattacatgagcttagaaatgtaa
ttagcatagtgtgtggcatagtgtagataccaaataaatatgatctctccttctactcttgaaaatgcaaacac
attcttggtggtcctaaaatagcctgtaacatggtttactcagcagcatttgctattcaaggcagatctgcctt
tagtcattggctgcgctcctgaacagctgtgtgaaaggctaacttttgtaaaccaaatcaaaataaaatgcagc
aaaaatttgtcactgaaaggaaatcctcagtatatccttttatgaaatgaaagatccctcatccaaacttaact
tttttaaaagtgcgcatttggagatatagccctttcttatgaatcctaattcaattttggccataaacacacgt
tgatgttccccaccccaaagcacatagcaacaagagtaggttctatattgaaaataatgacaatttaaaaacat
gtacttatttcactgtatgtggacagtgtctatgattgcatcatgaagtgtcatataaccatgtacgtgtacat
gagagagagatagagagagaagtggtagggtggtggtggtagaggggatggcgatagtaatcatggtaatggta
gaggtgatggaggtggtaatgacggaggtaagggtggtagtgatgatggtggtggtggtaatggtggtggatgt
ggtggtggcaattgggatggtgggatggtggtagccatggtgatggtggtaatggtgttgatttaaagggtggt
ggtagtgaaggtgagggtagtggtggtggaggtggtggtgctggtagcaatagtgatggtggtgatggtgttga
tgagggtgttgggatcagggtgagttcccacagtatatttcattcttgttgtaccactctgtcaacagcaccac
tgactgggacagaggaagaaggcacactctgaatgtgttattaacagaaacctcaaaacagtctgtctccttgt
agtcattcaaaattatctttttcttacctggaaaactgaaactgaattaccgggaaaaacacaggagatttttg
tttgttaatatgctgccaataaagtaattttatgtcaaatttaactacaggaaagggcaaggcattttctaagt
tcettagatgtcatgtggctaaaaaaaacaaaaggatggacageagttagatactgtacacttagctgtttgaa
gccatatattcagaaagcagatgttgggagttggtgtttgaggactgatttcctggaggtattttatataggcc
aagttcattgttctaaactctaagggcttgacttgagggaggaaaagaggcaagaacatgtttagttttgctga
cagcatcacatgggcagccctaaggctagacaactttagggcctgaagcttattctaggaaagaagcacctaca
gagtggcactgggctcccctccactatagagatgaagtcatatgacagtaaagggcaggcagggctgcctaggg
ggcccagaactgacacttccattagaatgagcacaggccagggagagaagtggggaaccagagagaaggagctg
gaattctagtaggacaaacggtaagtgaacaacaagaacaagttaagagtgtgtgcagtattctttcaaagact
gaaaaaatagtgatgtgatagaatggcaggtggctctgagcaggccaggagaaggactgggggcagagcatccc
aggcaggagggcagcaagtgggaaggccctggggtggggcttttggactgttccagtgacgggcaggcagccag
tgtgcctgtcacacaatgcaccagggaagtagtcgtgaatttgcagagggtcttgcaggctatgggaaagggat
tggattgtattttgtttgtagggaagccatcgggggacttaagcagaggaaggattggcttcatctctttgaaa
aagttctctctggatgetgatgggaggagaaatggaaggaaaagaaacacttttaggggcaagaacttttgaga
agggtggaattgggagtgtggagttggggccagctttggcacaggaggggaagctaaacacgtggccgcatgag
ggcctgtaattctacctgaaatgggtaccatttgttagggtaaacaaatgaaccaaatgcccagtgatacagac
caagtgttggcaaacttcttctgtgatggcccaggtagtaaatgtctcaggcttcgcaggccatgtggtctctg
ttgaagctctgtgtagtagacaatatgttaatgactgggcgtgactgtgtgctaataaaagtttatttacaaaa
acageeegtgggetggatttageteacaggctgtagtttgccaacctctgacctagagcatgaactgagcatct
tcttggagggaaataagttctttccaagttgccctcctcacattgcagggggccatgtaggcccattattcaca
gaagagtgggtgggcaacctttctggagcagaaaaacgtaaagatttcttccgtagtgcaagtaaggtgaccat
ttctaaaccgtgcaagtgatccagcagtcccaaaagttgtttcacttctcattgtgcgcccgttctcaggtgct
ccgaagcttccagtcctttgtagggacatggatgaaattggaaatcatcattctcagtaaactatcgcaagaac
aaaaaaccaaacaccgcatattctcactcataggtgggaattgaacaatgagatcacatggacacgggaagggg
aacatcacattctggggactgttgtggggtggggggaggggggagggatagcattgggagatatacctaatgct
agatgacaagttagtgggtgcagcgcaccagtgtggcacatgtatacatatgtaactaacctgcacaatgtgca
catgtaccctaaaacttaaagtataataataaaagaaaaaaaaaaagagaggagagaaacatcatcccctccag
gatacccttgggccttgttcttatagtcttgtacattgttgaacaatttgcatgggetagtggattaaagcaca
ccctccaccctcaggccctcaagggtctctatgataatacagtctcaccttctaccctttccatcaccatccta
ggtgctatggccaaccttgaggctgccatgttaggtctatgcatttcccacctccaccacataactctctgaag
gccaggtagtttcctattcatcttggtaaccccaaagcctcgtgacagggctcagctggcatctgcggatgtga
atgaaccattggagaaaatggtactctgcaaataactctgttattttcccatttcctgtgtaaggcctagagac
aatgactttttaattgcaccccttcccctctgtatgacactggccttctcttgtgtccagcaatgtgggtggcc
tagatgatttctaagggacttctggccaagatgaacagcagctgcatcttactgagcatttactatgtgccata
tactcagecacagetctaggggcatagaagcaggagctctcagggtcagggcagtgagtgageaagcgageace
tatgeeagecctgcctctggatggggacttgagagggtgatggaagcctgcagcactggagggaggcagacaaa
gacaggcctgtgetgagggggcceggageaagagagagggaggcaatgacagcagagacatgcetgcgccttgg
gtttgagtgcccagtggtcaaatccacttccctgtggctgatgcttgcctttctaactttggaatttaggggtt
ggagatctggtgagaaggtaggagggagatgaggaggagaagggaaaggcaggaaggaaggggagggaaaggaa
aagcaaaaggggaggaggaaggtttccaacaaattattctatatcaactgcggaaatcaaaatttgttgcccaa
atcttagaagctcatgtccctcctccccagaagtctggaatgcagcactccaggggtagcttataacccaaata
tetatctgtaaaaagagaaacattgggetttcgagctgtggattctcagtaaaagcaagaggcctcagcctaca
caggccagcccagagtttgaggaaccccaggcccacacccacagggctggcccctgggtctgcatactccctag
aaatgtgcacacttctgagcctcaactctgtcctggagtctaacagcatccctctccttcctggggcagttcca
cctccagaaacctgttaccttgggccttatgtcaaggaaactgtgggaaagagctaggcaggaatgcagatgag
gccagcatgggetcctaaaagtttagaaataggcagtgtcatgetcccaggtgcctgcataaaccagctgaaaa
atggagctcccctcaccagcactctcccttcaaacagactgtgatttgcaggtcactggtttaccaagccaggc
tacccaggcaggacccagatgccaagcccagtggtgtcctgcaagctgagcagtgctcagttcttgcaaaaaaa
ggtctgtgtgaaggcaaggcctctgcctggcttctcaccccagttgggtgtctggaacaggaaggagcccttac
tgcagaaaaaggaggagggagcaaagggagcgaacagctgcgtgctccatggggaggatccccaaagtagaaag
gcgcatacacactgcagcccttgacccagaatgctcacagctacattacagattcaggtctcctcagtgtagtg
gggctgctgatgagactgtggcatcctcaggggtcaggacacacattttccatcactcttctgatggcaaaaaa
cctctgagccaatgccaacctctgatcattaaaaaaaagtgctcacagcagtgtgtggtttaggatcatgccct
gtgtggtttggaacacgtgcacaaccacaccttgttcatcaccatcccagaaaccctgacgcaggcaaagagca
gagttattaaccctactttactgatgtggatactgaggcccagaggctcatgcaagttatcaataagtggcagg
gacagttgcctctagattaactagcccctaggatcacctgggtcttggaaggggacccataaacatgagctccc
ctctcttggggccagatttgcacctgtgccgcgccttcagectgcatgaagtaggggctgetggcaaagactea
aagctgtaaatctgggttttctcttgaggcttctaagggagctgtttcgacaactcactctgttcccagctggc
tgcccctgcatagggttttaaagcagcctagctttctgccaggcttggcagtggacaacgctggtcagaacatc
ccagagagctaccagaatgaagtaagtttgcttctactctttacctgtttatgggctgtctctgccactggaat
gaaaggcactgagaacagtgcctggcctgcagaaggccctggaaatacctgagctcctaatctgggaataggag
taggaagagctttggaggcagggcacctgagtttgagatctacaacttcctgcctgtgtgacattgggaaagtc
tccatcctttctgagcctcagtctccaccctggggaagtggaaatatcaatctctgtgacacagaagcaaatga
gcgaatgtgcacaaagtaccttgcacaagagagacgctcaaacacttgcctccaggtttcaccgagaactacag
agtaagatagatttgttcccagtggaggaagcctgggaataatttgcccctagactatgaattcctggggctca
agatcgagcacagggccaggcacacagaagggaccctggaaatgtggcaggaggccagagatagacaggccctt
agagctcatacccatgccctctgacctcaagaagaaagaaacctgctcaaaatctcacaaagagcttgttccaa
ccctgaatcgagtctgaggactccttcctgagtccagcactttttctgcaagaagtatatgcctccaaagctga
tgggcgcaaatcttgaaccccgtcacataaacacaaagggaggaggtgactagagctcctcctactggatatgt
ctaaggtcaccagtctaaagaaaagggatggatagaatgaggccagtatttttgcagccatccaaatgtccaca
tacgctgttacactgagggctcctctctcccccgtcttcagccctacttgcatttagaggtgagaaagatatgg
gctgaggggttgtttttcatcgtattgtagatggaaagcacactgcccttggggccatccaaatgtggaccttg
atgtagcaccccaccttctggatggccatccttctgaaagtcactgaatttctcagactttattctctttatcc
ataaagaaggagaataataataatccccccaccctgcccaaccactgactggttgggaagctcagaagaaatac
tgggcacggcatcccattgtaatctatagagtgagtcgcttcttaatattaaatggctgaacacagaagatgtg
caaaaagtactgtgtccccttcctcctccaactgaacatttcatgccctttgcaccctcattttgtctaggagc
tgccttatgaagggaataggtacctgctccgagctggaggaatctttgccacttatggtggggtatggactgag
acagagatggcatgtgacatgcgcactgagtctcaactccatgcaggctctggagcactctcaaattggagtac
taatgccttttaaattctcacactagcaatcctttgacctactgatctagggatctagggaaagaatcgtgate
ttaacttcaaagggaaggacaaaatgttctgcctcctgttaaaactccatacactaagtgcagagactggatgc
cttattaaccttgggtagatgcccaaatgttcaaaaggtcaaactcttctgttccccagatcgccagagtcatt
aaccagtcacactattaaatgaatgaacagatgctgaaaaggtacttgcattactgagatttcttatggtgatg
gcccctgcctgatatgtattcagcattttgtagttttcaatgtgcattagagtatagtggtgatgacattggcc
tctgagtttgccacttcttatatctgtgactttggtcaaattgcttaatctctctgagtctcggtttcctggag
ataataatagcttcttcttcccagggttatcatgaggattacaggagataatgccccaaaaatgcttagtaaag
tgcctagcacctagtcaatgctgaattaaaggtggttattcttacttttcgttcatttgaactttgttctcagg
gagggcaaaggatagacaaagccccatagctagtgaggagtagctgcaagactagaacccaggtgttctgagcc
ctagtcttaggccaagaacaactgttacgtgagatgcacgttttccttcaagggagctcacaattatttccatg
taaattcaaggactgctaaaagagaactctcctctgggactgatatcattttatttcaagattgatttgaaaca
tgttttttgtttgtttgtttgttttctaggaaagaacaagagaaccagttaagctgaatgcctgaagcaaatcc
ctgttagcgatgttttcaggatgagggagagtggtgcaagaaacgtgcttccagatgcacatggtttcctggga
ctagggttcagggtgtcatccctgggtgttattaagtgtcagaaggagagcaaacaagggaaacatctgagatc
cagctaaggctacaccctggaaatgcaagcccagctcttgcaaaggacctcctttggccactcaccttccaggc
cttacaataacttgtttggactgcaggtttcttggtggactcacaggccattctgcttttatttggtcaacctc
agttcacaagcacccagatgetgagatcctcagcatgtgcagcagagtttcatattagcactgggtacctttct
gaggctacagggataccgtacagcagcacctgtcacgtccagccaaaggagtgggctctctcaatgtcatccaa
tgctgtttcaactgtgaagaagaccatctgagagagttgcttttggaggctgaggcaaatttttaaaattcttt
gttctcctcaactggggtgaattcttggtcttctaggacagcttgaagttttagaaagagtcaagccactcaga
accaacagagaactctttcagagaacaaggtgtggcatagaggaggcagagggctgatcttgatcaaatccaaa
gtgtgactctaaagcaatgaatgtgaatttttggcaaagcttacaaagggctctaaaggccatctgcaaagaga
agccaagcctgatcgatgaatcactagtgcggccggatatcgatcggcacgctgttgattttctcatagtaagg
aacagtgggccctttcagtcccacttctgtagtctgtggtactacaaatggtgagcccatgatgttgccattca
tagggttattctccagcagtaatgactggccagccactcccatagccgcggggctaggatttattgtcaatgga
gggacctgcagttctgcacaagcagtactaggatgagcacctgggcccattgcaagggtgacatcttcaaggca
aggcctcttaattttattagggtagcccccatcagecatgtctggaaactggaagtggtcttcttcttgtctcc
tcttaacagttccctgtgaatggaagagaagagaggaggagaagagaggagaggagaagggaagagaggtgaca
cacacacacacacacacacacacacacacagagagagagagagagagacagagagaaagagagagagagagaga
gaggaatttttataaaggtttggcacattaaagctaatgaacaggaaatgtgcatgataaaacagacctctcag
tttaaagacttatagttgtgaaaactataaaatacagcctgtctttggaaccatagtgcttatttattcattat
tatgtttcatctaaactgtctaattacatttcaaataaggcattatgttgtctgtatactaaaacgggatagaa
cgttattcaaagggtaatctgcccacttcaaggagagttcaacaaaactatgcagaagtcactaaatgaaccat
gctgccaaaggcaggcattggagagaaaactagaagtagctaaatagttttaattctttcctgtctacagacac
atagattttaacgaaggaataccatagtatagaattgaacttttaggctgccttctagtcttggttaaatgcat
caggctgcagtggtaaaattgaatacaacagagcccttacaggaaagaagtagatctggatgtgttttcttggg
gagctgtttaaaatactgtttttgggaaagcacaagtttcagaacagtcattgtaggcatcgtattcattgttc
catttatttttacacacacacacacacacacacacacacacactctcacacattgctatgtgtacacaaaaata
atttggaagaacctatacccaacaatttggagtggtcatttatttgggatgactggcaattccctttctattct
cttcatttctgcttgtttgtctttaacgagaacgactcataatccaaaaatttaaaaaagtataaagttatcta
aataagaaattttcctctgaagatgcatcctcaggttggggagatattaaacaatgagaaaaggccccaatctg
ggatctgaaccttgggggagctgcccatcatttatagaagcacagcctttgggaacaaagcaaagtcactagca
atgtgagacttcctactcttcatggcttcatacagtcatccatcgctgttgtgttaatgaccatgacctgtatg
ttagcaggtaaatgggaaaggaagtgggggcaaaggagtatgtgcaggaatgatcaaaataaggaaaggaagag
agggatctggaaatcacctgaatgccgataggtgaacaggtagaattcttttaaagcttcccccacccggtacc
ccccaaataacccctttccagctttggaagtttcactaggacatacagtgctcatcctctgatgtcaccttaag
tttggctcttctggtttgatgagcttgtagcccactaggagctcaaggcatgcatggggccacttgccagcacg
atgaggggcatgactgtcatggccaagtgaacatcaaagcagatccccagggctgtatgtctcaggccttggtg
cacatcagaatcacttagaaacatccacattcctgggccctcccaccacaaactgacagcttcatccagggtgt
ggcccaggcatcgggagtttttccaacagctccatggctgattctcaacagaaaaccactggcccagagcaagg
gtggaggcagcgtggcatagggctctgaccttggccttgccactgaacctctcagagccccagtttctttatgt
gtaaaatgagtgtaattatagttcttttctcatgaaggtgctctgactattaagtgaaacggggcacattgtat
gacacctaatagctcctcactaactggtacccggcattataaagggcaggtatggaagggttctgggagtccaa
tacccttcttaaagacagagaggtctctgagacccagagaggggcaggccttacccagagttgctcagccagag
ggcaacaaggcccaggtcagatgcagggcccctccaccaccactcagctgcctccagacccactgccttcgcca
tgttgttggtaggacactgcatcgcccccacagaaggggcttgccaacttgagtgagaggacttgcacacttct
ttgacttttcttttgagatgcccacaatctgaacaagggcacttcaagggacagctctgtcaccaaactcatct
gaggcctgaataccatgggtcaggcaggaatgggttggagaggtgtagagcaggcacaataagagggctgaggc
ccatgcagtcatcagtgcccactttcccaggagtctgactgggcacagcacccatagtgtccctgagctggtcc
atggagcagctcactaactgtttggcccacagcaggtgctcagtaaatggcagttgaacgaatcaatggacaaa
ggaacataaattacccaacacacagggagctcagccatttactcaatccattatggagtaacctacaaacaagc
cactgggtcccaaactgaaattgtgtctcttctacattctcccaaagaatccaataggttaaaaatagaaatgt
atgaaatagatcaatcagggatgattgcatgtggatttgacataaggatcccctgcagggagtctgagctggca
acagtcaggcccaaagtgctgtccatgatgtctcgaactgcaagacagttttaacaatggcgaagcaatgcaga
accaggcaggccaaggagggggtgggggttggggaaaggaagggagggaaggggctgtgaggggcaatggtctg
gcatccctgccacgtgagcctctgaaatttgctggcagcttctatgggctcccagagctttcacttaattgttg
gtctgccactaacctgctgggagtaaggtgcagggatggaggaggcagggcatgaccaccagacactaaaggta
ccagctggggccactggcaaagggaaggaggctgcacctctcctacatgagagcccgtatacacacaccttttc
cagcactcatcaactgcatcccaagcaaatggtccctgatcaattccaattctagaaaccaactgactactcaa
taacaaagtagatcccagcaggccgccactgctggagcggatgccacttttgctatgccaagtctgtggctgga
cagctgctggcatgtacactcactgactttcataaggatgcctaataaagggggcaggctcacctggcttttct
caggggtggggtttggggtgccgatagaggctgctgttttggcagagtggcaagctgcaagcctcttctgagct
ttcatttttcaatggacttcagtgagaattcactttgtcagaggccatgcagctccatgttttggatttcatgg
aatgagctttcaacagtgagectgaagtgccctggctgaacageaagaacaccagecaaccetaaacaaggceg
aggagaggcggctgtgtttacacggaaggctcagccttgctgtaatagcgtctgccttcaccagacatcagtga
ggcgtggaaatctattatccagttaattttgcccctagataaagacttgctttcgtgtcttctctttcacagtc
ccatgatctgttactcatctcaactgcgagaagttggctgggctttcccctgtgcccagtgccacactcgtgcc
ttcactgggtcacctgtgcctgtggctgatgccgctgaggttttgcctgcccagactgggtgtttctgactaaa
tcccacagccaccattttagatcaagggcaggagatagctcactgctccggaatgacctcccctcccagaatcc
tggtaggggcggaaggtccccaaccaagctcccagccctttctaaatgaatctccctgcttcacccatgtgctt
ttctccagtctctgcggtcttgatgacagcagggtattagtcctagctgtcccacagctcctacttctttcagg
cctctccctgtgacaatcagtagccactggcaggatttcctcagagcatatctcgatttgctttcagacaatta
gttaaaaggacactggaccccagacgtcccaactcccagccagagccctcacaggcccggcctttggtggtgag
gaagggggagggagtgagtgacagtgccctggcatcttttagaaacgaattcctttctctccatacataaatgc
ctgcagagtcccatttcagaatccggcagacaaagccaccaatgtgatccccatgaccttataaacattcatta
aaatgcatttcaaggcatgtgatggcctccccaccccctagataatgagaaaacaaaggtttctcttctgatag
agacaagttcagctctgaagtcaacattatttctggttctgtctgaacaatgacatatggcaactcttcccttt
ctatagttctagtccagaatgacaaaaaaggggaaaaatttcttagagaaggtagagattatacgaatacagtc
catgaaatgagcataaggagaataaagaatataacttatccaaagaagtctggcaggctgttataaatgcttga
ttttggacactgtagttggaggtttaacatggacaccaataaaaaggtcagcaaagggtatgcactgttcctat
tgggcaagaagataggaggtcaaaggtaaccaggaaagataaactcagggagacttattttccctccagagggc
actgggcttgtaggccctgggcaaaattgtcaaaaaggtgaaaatcgcctgtggtttatttagtctgctctttc
ttcactagtgcctcaccagttcagttcaggccaatttgctagaaggtagcgaacgatcgaccggtgaagttcct
atactttctagagaataggaacttcggaataggaacttctacetagatgeatgetcagagcggcccctagetag
cgtttaaaacctacagttgaagtcggaagtttacatacacttaagttggagtcattaaaactcgtttttcaact
actccacaaatttcttgttaacaaacaatagttttggcaagtcagttaggacatctactttgtgcatgacacaa
gtcatttttccaacaattgtttacagacagattatttcacttataattcactgtatcacaattccagtgggtca
gaagtgtacatacacgcgcttgactgtgcctttaagcttttaattaagagtaattcatacaaaaggactcgccc
ctgccttggggaatcccagggaccgtcgttaaactcccactaacgtagaacccagagatcgctgcgttcccgcc
eeetcaccegeeegetctcgtcateactgaggtggagaagagcatgcgtgaggctccggtgcccgtcagtggge
agagcgcacatcgcccacagtccccgagaagttggggggaggggtcggcaattgaaccggtgcctagagaaggt
ggcgcggggtaaactgggaaagtgatgtcgtgtactggctccgcctttttcccgagggtgggggagaaccgtat
ataagtgcagtagtcgccgtgaacgttctttttcgcaacgggtttgccgccagaacacaggtaagtgccgtgtg
tggttcccgcgggcctggcctctttacgggttatggcccttgcgtgccttgaattacttccacgcccctggctg
cagtacgtgattcttgatcccgagcttcgggttggaagtgggtgggagagttcgaggccttgcgcttaaggagc
cccttcgcctcgtgcttgagttgaggcctggcctgggcgctggggccgccgcgtgcgaatctggtggcaccttc
gcgcctgtctegetgctttcgataagtctctagecatttaaaatttttgatgacctgetgcgacgctttttttc
tggcaagatagtcttgtaaatgcgggccaagatctgcacactggtatttcggtttttggggccgcgggcggcga
cggggcccgtgcgtcccagcgcacatgttcggcgaggcggggcctgcgagcgcggccaccgagaatcggacggg
ggtagtctcaagctcgccggcctgctctggtgcctggcctcgcgccgccgtgtatcgccccgccctgggcggca
aggctggcccggtcggcaccagttgcgtgagcggaaagatggccgcttcccggccctgctgcagggagctcaaa
atggaggacgcggcgctcgggagagcgggcgggtgagtcacccacacaaaggaaaagggcctttccgtcctcag
ccgtcgcttcatgtgactccacggagtaccgggcgccgtccaggcacctcgattagttctcgagcttttggagt
acgtcgtctttaggttggggggaggggttttatgcgatggagtttccccacactgagtgggtggagactgaagt
taggccagcttggcacttgatgtaattctccttggaatttgccctttttgagtttggatcttggttcattctca
agcctcagacagtggttcaaagtttttttcttccatttcaggtgtcgtgagaattcgatatcccaccatggaca
aggattgtgaaatgaaacgcaccacactggacagccctttggggaagctggagctgtctggttgtgagcagggt
ctgcacgaaataaagctcctgggcaaggggacgtctgcagctgatgccgtggaggtcccagcccccgctgcggt
tctcggaggtccggagcccctgatgcagtgcacagcctggctgaatgcctatttccaccagcccgaggctatcg
aagagttccccgtgccagcgcttcaccatcccgttttccagcaagagtcgttcacgcgtcaggtgttatggaag
ctgcttaaggttgtgaaattcggagaagtgatttcttaccagcaattggccgccctggccggcaaccccaaagc
cgcgcgagcagtgggaggcgccatgagaggcaatcctgtcaagatcctcatcccgtgccacagagtggtctgca
gcagcggagccgtgggcaactactccggagggctagccgtgaaggaatggcttctggcccatgaaggccaccgg
ttggggaagccaggcttgggagggagctcaggtctggcaggggcctggctcaagggagcgggagctacctcggg
ctccccgcctgctggccgaaacctcgaggtgaaacagactttgaattttgaccttctcaagttggcgggagacg
tggagtecaacecagggeecatggtgageaagggcgaggagctgttcaccggggtggtgeecatcetggtegag
ctggacggcgacgtaaacggccacaagttcagcgtgtccggcgagggcgagggcgatgccacctacggcaagct
gaccctgaagttcatctgcaccaccggcaagctgcccgtgccctggcccaccctcgtgaccaccctgacctacg
gcgtgcagtgetteageegetaccccgaccacatgaagcagcacgacttcttcaagtccgccatgcccgaaggc
tacgtccaggagcgcaccatcttcttcaaggacgacggcaactacaagacccgcgccgaggtgaagttcgaggg
cgacaccctggtgaaccgcatcgagctgaagggcatcgacttcaaggaggacggcaacatcctggggcacaagc
tggagtacaactacaacagccacaacgtctatatcatggccgacaagcagaagaacggcatcaaggtgaacttc
aagatccgccacaacatcgaggacggcagcgtgcagctcgccgaccactaccagcagaacacccccatcggcga
cggccccgtgetgctgcccgacaaccactacctgageacccagtccgccctgagcaaagaccccaacgagaagc
gcgatcacatggtcetgetggagttcgtgaccgccgcegggateacteteggcatggacgagetgtacaagtaa
agcggccgctcttcgagcagatatcataagatacattgatgagtttggacaaaccacaactagaatgcagtgaa
aaaaatgctttatttgtgaaatttgtgatgctattgctttatttgtaaccattataagctgcaataaacaagtt
acgttaacaacaacaattgcattcattttatgtttcaggttcagggggaggtgtgggaggttttttaaagcaag
taaaacctctacaaatgtggtattaattaaagatctttaaacaatttaaaggcaatgctaccaaatactaagcg
cgtgtatgtacacttctgacccactgggaatgtgatgaaagaaataaaagctgaaatgaatcattctctctact
attattctgatatttcacattcttaaaataaagtggtgatcctaactgaccttaagacagggaatctttactcg
gattaaatgtcaggaattgtgaaaaagtgagtttaaatgtatttggctaaggtgtatgtaaacttccgacttca
actgtagtttaaaaegggecegtagtctagggcegeeagtgtgatggagttcggcttcaggtacageacactgg
cggccgttactaggtagctagagccttcagactctagggaagttcctatactttctagagaataggaacttcgg
aataggaacttcacccatggcgategetagectctaactcctagaccgtcagaactgetgggccettcaagacg
ggctgctcacacccactcatgttaagcctggtgaggcctgtactctgttttcacaggaagaaatcctcacccag
tcttccccaaacacattcccaggttgtgtcattagtgggatagagatgattattgtggggagaagagaaacatc
tggatggatttggtgaggttgatctatagaggaagtaggtgctgcctgaggtagctgtaatagaagctaaaggt
caaaggagagggccctgtcccaatccagatgactccacttctgetggacccaggttcacaagcttaatctacat
ttcacctaaatttggctaacaagcccaaaatcacacaggcaaagggagaagtggaggcagaaccgaggttggag
gccaccagggccaccgggcagagatcatttaagcccaaccttctcacttctccctgggctctgcctctcttaaa
ggaccttgtggtgtgacctcttgtaggtccctttcacactcggggcctcagtttccccactgtaaagtgaatgg
gtcccagctttggtaagettatgettacctgatgetttcttcctgggctgctcttgtagagaaaagataaatct
tcttcctccatccacgagggctcctttccctgggggtgagagtaggctgaggagagccacttgcacacaccctt
aaagaaagtattacctgcaccagctcagtgagaggcacagatcagactgttacttgaatcaaattatgagcctc
cccaaatatatctatgacatttaaataggggattacttgaacatagactgtgggatccggtgtggagtgcggga
gactagcaaagtgaatcctgagagtagcaggtctgcacctgttggatcgagaaaggcggcctacaattctggtc
aaatgagctgtgcttattgacatattctattagagagtactaccaggtcaccagtcaccagaaaggctgccagc
tctccaaccacctccagggaactatcctgaatggggccttaacaagtctaagagagggttggtttgggtcccaa
gccaatatttgctctgctttatgtcagtcatatggaacccaaaccaaccctctcctatgtgcctcaccagtcgg
tgcagggatcccaatttcaagtttggttttttatggtcaaagtccagcatagattaaatgaaggggtgtgatga
tggtgttaaaagagaactccagaccagtttaactcttggacacacatcccatctcaccatggtgcttccaacct
tccagagatgatgggctcctattttctgatgacaaagccctccacaggattgctgcctggccatcagggagtgc
ctctgtaactgaggctgagatcccactttcagtcctccagctgtggcccatccctgctccgcccaccgggtatg
gcctgtcctaggctcttaggtatggctgcattgtgaaatgatggctacagagctggcatctcctgtagtctggt
tcatctagtgcactacctcatagttaaaagaaatctgtttaagccactgagggtggctcctagtgccaactcca
agaacaggaagcttcccttttttgggaggaggggcagatggtaacatggatcgtccaggtcaatgggagcaggg
caaccacagtaagtactggacaacaacacaaaactccatgtgtggcttccatcgagtccctctccaattggttt
ggtcttctccgtcccatgcagcactttagcaaggggcctggctgaaggctatgaattgtgtggagcctcctcat
tgcagtctccaaccatctgatgctgggaaaatgtcaccaggatgcagccatgccgtgtggccaatgaaccgaga
aaacaccccttttctagaatgctctaaagaggcagaataatccagaggtgaggaaggaaatactccaccagaga
cccaggcagttcctacaaaagccagactttccttcacctagggagtgacaagaccagtggaaaacactctcaag
cagtaacccccaaatgctctgcaagccagtggcgtccagataccgcacaagcgagtgggctgtctaatcccatc
atcatgatgtaaatatctctaggctgccctgggctgtgcctgaccctgtcttcagctttccacacctccaccta
cagcccatgcacagaaggaccacccaggaatgctgcaagtgtggcacctccagggccacccagggagaaggagg
gcagctatgctggtggctccaggcccatttggcgggtggtaccttcacaccacaaagcccaaactgaggcccca
gatttggctgatgagggcatattggacaggggtcacttatgctcttccccattgccacctggcctctggctacc
tggacttggctacctgtggatcctctcacaggtgccaccatcttggctgagtctccagatgcgaggtccctgag
gcagtggcgggcttctcgctaatgctgatgggattaggaatgggataggtggggagggccctggactgggccct
gatgagccaagtgggtttttagaggggctactggtacatttcagggacaggacatctggtagagctaagctggg
gcaataaggagccactgctaatctgagagctagaaacaatcagcttctgggtcattattaattagggtagtttg
ggctgtgtggaagtcacgtactatatggggtagccacagctctctctacagataatctctaagacttctgattg
ggactgtgtgaatgcagtagcaatatctcttcttactgccaggccctgccagtcctgcctccacgccctggctg
gccccccttatgatctgacccatgccaggctgccatagtatgttacttctgcattagcactccttgggacctgc
ctctccactgtccctcagactttaaagaactatacaaacccaaggggctcttcccaagagaattgatatgactt
gaggtgattccatttctggaagtagtcactccattttctgcctcactctttcagtgcttcacagagcaggttcg
aacgaaggagccatccaactaaccgtcatgttcgggcaaccgaagaagggagtggcaggatttcctttggagac
ttctggaattagacagcagtttaatgcaagcatctaaattctctccctcccagagtctcattaaaactacagta
agagtttgtgttttgttttgtttttaaagacaaaatcccaccaggatagagagaataggagaggagataacagc
atcataatttatgaaactaaaatgcagatagaccaggattaactgactacacagcaccaaggaagctgaatcac
aagacagcagaggagaaaactggaaaggatcgtggtctatacggcagaatcttcccaagcctcaggaggaggag
ctctagatgttcccagatctgggaggtaaagtggaatggggggacatggtcagcgtaatggggttgggctggaa
gcaggttaaggagcaggcagatctctgaatcccctctctgactctgtgtccccaggcatctgcctgtcccccac
cctggaagaggtctggcttgaccctttgtctggtgaatttcctgctctgctttcctggtcctgctggccggatc
agtggaggccactcacttcaccccacagggatgttctgtgttgccctacacctgggaactggaggtactggagg
caggctgtggtgagcttgaaagcaaaacacagagggcagtccaatctctttggccatatttcttctgcatatcc
aataccatgtccacaactctgctagtgtcctgatggtggtgggctctacacattcccgggaagctgaaggcaga
taatgaccaggacaggtcaacctctcttcttctgaaagccttcatctactaatggcctgggactcttcccttaa
atgcttagattgtgtcttccactaaggttttttgctgttgctgttgtttgtttgtttgtttgtttgtttgtttg
tttgttttgagacggaatctcactctgtcgcccaggctggagtgtagtggcacaatctcagctcaccacaacct
tcacctcctaggttgaaggggttctcctgcctcagcctcctgtgtagctaggattacaggcacatgccaccatg
cctggctaatttttgtatttttggtagagacaggatttcgccatgttggccaggctggtcttgaactcctgacc
tcaggtgatctgcctaccttggtctcccaaagtgctgggattacaggtgtgagccaccacacccggccaaggtt
tttgtttgtttgtttgtttgtttgtttgttttgtattgaggcagggtatcactctggtcacccaggctggagtg
cagtagtgcaatcacggctcactgaaacctccacctccctggcgggctcaggtgatcctgccacctcagcttcc
caggtagctgggactacaggcttgtaccaccactcccagctaatttttgcgtttttagtagagacagggtttcc
ccatgttgcccaggttggtctcaaactctgggctcaagcgatctgcctgcctcagcctcccaaagtgctgggat
tacaggtgtaagccaccgtacccggccccgccactaaggttttgaaaatgaagcaattacaagtttaagtctat
taataagtgatgaagccatgtagaaaagcagaataattatcttggatcaggaaggtcacatgaggatctacttg
ggggttgtcaatattctatttcttgacctgatcagtgttgacagcaggttttaattttttacttctttttgttt
gtttgtttttgagacggagtcttgctctgtctcccaggctggagtgcagtggtatgatctcggctcactgcaac
ctccgcctcctgggttcaagctgttctcctgcctcagcctccccagtagctgggattacaggcaggcaccacca
cgaccagctaatttttgtatttttagtagagactgggtttcaccatcttggccaggctggtctcgaacttctga
tctcgtgatccgccctccttggcctcccaaagtgctgggattacaggcttgagccagcgtgcccggcccatttt
ttacttccttattaaactgtacatataggccttgcacacttttctgcatcaatgttatattccacaataaaggg
aaaaggtatatacacaacttgataccagtaatgtgaaacatatatttctacatagaaaaaaaaatgactgaaat
actgcactccaatgtgttcacacagtagttgtttctggattatttatatattaaatgtttatatattgtattat
gccatgaggtttgtgttttctctccacttttctgcattttccaagtttactacaaagagcacatattactctta
taatcagaaagtcataaaatatatttaaaaagacaaaattgaaactaataaggatcaacacaaaacagatgagc
catctgtggaaatccgcacagaatactacctaaagagattggtgacgtgcatgatctcactaggatgagcacaa
agcttgccagagcctagggtctatttctagggttggctcttggaagccaggatagttgttatctctgggaagag
ggaggggcacacaaggggcttctaaaacattctgaatgttctatttctgaacctggttggtgggtacatgactg
ttggttttattattatatgttttatatactcttccgtatgtatggtgtggattccaaaaaaagatttcctttag
agaaaaccagaatcacataagtagaaaatatggtgctatgttgaaggaacaactcaagtttatataaaatcatc
atcatttataggcttaaaaagttgctttggaattttggtctaactgacttgtcttttctgcagcaaaccacgct
ccttctggacgtgctccaggcagaggggattagggtgggttcaaggctgcaagtacctagctcagcacactctc
ttcaggggacttagagtttgtctggtgttggctctctgagctcttgtcaggaatgccgacccttccgaggttca
ggatttgaagcctgccttcccaccccagatttggtccacacagacactcaagtatgtatttcaactacaaatga
cctgtactttcctattactcctctctttcatggtaacctttctggtatccttccttccctacatttatgggagg
gggacatcattctctgctctcctgtcactgaaggctccaccttctgtcttcttctgacccatctggttttcctg
gggccacctcctctccttaccaccctaacgcttttgtaacttgaggagaaatgagagatcacctagtcaggtca
tcattctctgtagatgaagaggcccaatggtttgctcaagaattgccaagcgagttaaagacagagagtatgag
agtcagcaagacctacagaaagcatctatctgcactgttttgcagggacttagcctttgtgtgtggactcctgg
aatgccacccactaagaaacattgtctgacaccaactccccacttggtaggtggggacactgaaactcatggca
ggaaagggccttgccccaagccagggcagagtgtcactcatcactctcaattttcagtccagggcaccttgttg
tgactatcccaaaggcagccactttccctggtctgaaagacctgaagagagaagagaagagaaggatggaaggc
agagtatgcggctttgattcatttcctggtgaaaacagatctatacgagaagcaaatttcacgaaagggaagag
aagaaagtgtcccatacgttgctggcctgtttcaaccttgctttgattcttgctgaaaagggtaccgtgtattt
ctgagttcaacatgcagaccagtgttaggaaagccactgcacctccactttagcctccagggctgtgccctgca
aatggcctgcagccttggtgcctcgctctccagactgcattttggaagatgggacagaggcttatggaagccca
cattagaacgggggagcagaatgggtgagatgagggatccttgatagtgaaccagatgaaggaatggtagccaa
atgccaggcctcctttgtggcttcaatccaaaggctctggagcccttccagggcagaacatcaggcatgtttac
ccccactgtcctcaacagtgacagaggtgcaatcttgggcagctggccattttgaaagcaacctccttaatctc
aactgggaaggctccctagcaggacccctgtgttgcacacctggaggaagctagactaaccagaagctcagcac
ggttccatctgggatgcccaggtctgagacgaaaaaggtaactctcttttctgggtcctggcccagttgtgtct
ctctccacctcattctctgagatgcctgtctccccttttttgtcccatcaggaggcaagagctatcactgggcc
agactccaccagaagccaagccagcttgttacccagcttctcagggagcaaagaacagccttgtttctatctta
tccccactgtcccctgcccctgccccacctcccagccattcagcttctggcttccccagagctgcctgcttctt
tgtggtcctccattccttgaaaagaccttctagtcattagtgtatataaatggccacttagcccagattacagt
gaggtcaacagctggggctctgagaattgtcacacactggcacaggagaggaggctattcttccagagaatttg
gagggcactcccatccacttacaacaaaaagcccatccactgtgcttggcagtaggtgatctgagaaccaatgg
aaccaggttaatcctgtggcactgttgagtgaggagagcagtggcgggcactggaaaatatcagagacaaggca
ggagacctgaaatctaggcttagctcctcatatacttggcagctgtatgacctcagacaaccagtgttacctct
ctaagcctcagtttcctcatgcaaaaggagggggaataacaacagagcccactgcttgggggtgttgtgaggac
aggatgaaaaaacaaacagaaatccctcagtacaggattcagtgcagtggacagtcttgcaaggtctggttcag
ccctccacccctaccctcaccagtataaagaactctggcctacaagtcagatgacctgagttttaatctcagct
ttgccattagccgtgtgaacttgagaaagtccctttcctttttacatctattgggatgatcatgcattttttgt
cctttattctgttaatatagtgtgttacattgattgcttttcatagactgaaccagccttgtattccagggata
aatctcacttggtcatggtgtataatcctttatacaaatgttgctgggttgagtttgctagtattttgttgaag
atttttatgtcttgattcataaggaatattggtgtaccttccccttttatggccacagtttccctacaatgatg
tagtcgaactagacaacctccaatatctttcagtattcatgtcctctgattctgtgaaactaagaaaattaaga
aatagtgattcataggcacaaggcaggcaaaacttagactccttgtagaataattaggaagccaaatattcagt
gtgcttatttctcaaataaccttagtttctccagtctgccccaactccgaggcctgaatatctctagatgctta
tgatggcaactaaagcctaaaagctaattcattttaaagttcttccaaatgcatagggttttatttttccagac
ctgggttcagatggggaatttgacaaacaatggaaagggggaaaaacaacaatctaaacactgagtgacaaagt
aacaaagaaatagtctagctatcagccagtcaagccagccttggctttgctatccaaagtagtcagtctaattc
taccaccagtttctgttcctgtagctgtctactgcctgccagggactctgccttcccacccacaactaccaatg
gaaggatgtggtgaccataccagtggctgctgacatctcctgccatgggaagcataattgcctccagcagcctc
ccccttagatccatcatttttgttgcacttggcctgggctgtactcccggccaatgactgaacatggtgagcat
agtaatgcaggcccatttctgtgaggagcaggactcctccagtaggtgactttggctcaaggactctctattgg
cctggttgaacttttcctgaactgtgctactgtctgagactcttcttacccaatcctctttctcgccccaattg
tcacagaccacctgcattgtggtctgagtctctccccaccttctcttgctcttccctgtttatctttcacaggc
atttcccccagtacattccttgaatgtctaacccgatacgggtgcctgacttttggcagacctaagcagacaaa
aaggagtacttggttacctagctcttctttctaccacaaacatcgagggaaccctttttccctcacccctctgc
cacacccccactgccccagtgaacaaccacagagagagctgtggtataatattaggctggtgcaaaagtaattg
cggtttttgccattacttttaatggtaaaaaccgcaattacttttgcacctacctagtatttgtgtccccccaa
attcatatgttgaaacctaacccacaatatgatgtcattaggaggcaagaccttgaggaggtgattagatgatg
gggtggagctctcctgaatgagattagtgcccttataagaagaagcccaaggaagctaccttgactcttccatc
acatgagaatgcagcaagaaggcaccatctactaatcaggaagagagctctcaccagacactgaatctgccagt
gtcttgatcttgaagttcccagcctccagaactatgcataatgcatttccattgtctctaagccacccagccta
tggtattttgtcatagcagcctgaactgactaagacagtgagccacatgagaagtgccccaacccctcccttaa
gcacttggctcacagatcagtgggttcatttctgcctgagttttattgttattctgtagatttcttgggctaga
tatatttttctgttattttccttcttcacctcagtcatgaattggttgttttaaaaaagacaatgtaagtcatg
gggaaactcctgacaactctactctcctagggttcctgataaaaggggattcagttgagtcctctgatggtctc
tacctgccaaagtccagcagcccttagcaaacatgetgetcgtttctgtagagaaggtgetggtgtcccaccat
acttctctctccctcatgaagggcttgcgacccagcaaatgggtggcttatatgggtctgtttcaaaggaagag
ccagctctgggaagaaaaacgatgagcataagcataacctaccactgtgcctgggaaagcagacaacttttttg
atgtgtgaatatctaatgagaatggaatccatcaattaccttaaacttaggcacagtcttcaaattcaatatat
gtgggatatacttttagtcagtttgtagacgttatttgtaataaataatctggcttctctaaagaaattatttt
aagtgtttggtttggtttgatttaatggtaaaattatatttagtggcagagaattatagcaatggtgataaact
atagagtgtcataagttcatatcttattctcacatttgaagctgcctgcagatgcattcaagatgcagccagaa
gtcaggagactcaggctgttatttggagctcatcattttacagccttgctggactcccactttctcaggggaaa
aatgtggtgttgacccagattagctctccaggccctgctgagttgggcactctgtaagctggagggtcttctat
tgtcttcacctaagtgtcaatcaacaacccaaatgggcatgggggaagagggagctgggccaatgcccagggtg
cctggtagagagataccttgggcactggaaggcaccagcttcccagagagaagggggagggccatgaaaaagtt
ggctgtagatgccagggacactgggactctccagctgtgtgtttgtgtcttctgaagacttatgtttcattcct
ttggagcatgcataatcatacactgtgggatgtgttatatagattgcttgatagttcaccactgtaataaaata
ctgtgactggaatctgctcccagtctgcctttgatagcacttgtgcaacacacatttactgagcatttacagtg
atccaggacctgtgttgtgaaaacattgatggacaaggcagatggtggagcacgtcagtgaggatttttaacaa
aggctggtaagtgctataaaggaacattgtaggacactagagaacaaagaacaggagaacctgacttaggctgg
ggtggggcgttggttagaggaggctccttggaggacatgaggtttaagctgtgacctgaggatgaatagatgtt
ggccaggtgaggtaccggtatttgtcagccttaccagtaaaaaagaaaacctattaaaaaaaaaatacacatac
aaagcctcatcagecatggcttaccagagaaagtacagcgggcacacaaaccacaagctctaaagtcactctcc
aacctctccacaatatatatacacaagccctaaactgacgtaatgggactaaagtgtaaaaaatcccgccaaac
ccaacacacaccccgaaactgcgtcaccagggaaaagtacagtttcacttccgcaatcccaacaagcgtcactt
cctctttctcacggtacgtcacatcccattaacttacaacgtcattttcccacggccgcgccgccccttttaac
cgttaaccccacagccaatcaccacacggcccacactttttaaaatcacctcatttacatattggcaccattcc
atetataaggtatattattgatgatg (SEQ ID NO: 190)
Exemplary HDAd35 donor vector (HDAd35-T4-Ef1a-mgmt-mCherry)
(Ad35 5′end: 1→481; FRT (Complementary): 14126→14159; pT4 LIR:
14220→14463; mgmt.: 15830→16450; 2A: 16451→16522; mCherry: 16526→17230;
SV40 pA: 17259→17380; pT4 RIR: 17491→17756; FRT (Complementary):
17863→17896; and Ad35 3′end: 29579→29986):
catcatcaataatataccttatagatggaatggtgccaatatgtaaatgaggtgattttaaaaagtgtgggccg
tgtggtgattggctgtggggttaacggttaaaaggggcggcgcggccgtgggaaaatgacgttttatgggggtg
gagtttttttgcaagttgtcgcgggaaatgttacgcataaaaaggcttcttttctcacggaactacttagtttt
cccacggtatttaacaggaaatgaggtagttttgaccggatgcaagtgaaaattgctgattttcgcgcgaaaac
tgaatgaggaagtgtttttctgaataatgtggtatttatggcagggtggagtatttgttcagggccaggtagac
tttgacccattacgtggaggtttcgattaccgtgttttttacctgaatttccgcgtaccgtgtcaaagtcttct
gtttttacgtaggtgtcagctgatcgctagggtatttaccggtattcaaggattacatgagcttagaaatgtaa
ttagcatagtgtgtggcatagtgtagataccaaataaatatgatctctccttctactcttgaaaatgcaaacac
attcttggtggtcctaaaatagcctgtaacatggtttactcagcagcatttgctattcaaggcagatctgcctt
tagtcattggctgcgctcctgaacagctgtgtgaaaggctaacttttgtaaaccaaatcaaaataaaatgcagc
aaaaatttgtcactgaaaggaaatcctcagtatatccttttatgaaatgaaagatccctcatccaaacttaact
tttttaaaagtgcgcatttggagatatagccctttcttatgaatcctaattcaattttggccataaacacacgt
tgatgttccccaccccaaagcacatagcaacaagagtaggttctatattgaaaataatgacaatttaaaaacat
gtacttatttcactgtatgtggacagtgtctatgattgcatcatgaagtgtcatataaccatgtacgtgtacat
gagagagagatagagagagaagtggtagggtggtggtggtagaggggatggcgatagtaatcatggtaatggta
gaggtgatggaggtggtaatgacggaggtaagggtggtagtgatgatggtggtggtggtaatggtggtggatgt
ggtggtggcaattgggatggtgggatggtggtagccatggtgatggtggtaatggtgttgatttaaagggtggt
ggtagtgaaggtgagggtagtggtggtggaggtggtggtgctggtagcaatagtgatggtggtgatggtgttga
tgagggtgttgggatcagggtgagttcccacagtatatttcattcttgttgtaccactctgtcaacagcaccac
tgactgggacagaggaagaaggcacactctgaatgtgttattaacagaaacctcaaaacagtctgtctccttgt
agtcattcaaaattatctttttcttacctggaaaactgaaactgaattaccgggaaaaacacaggagatttttg
tttgttaatatgctgccaataaagtaattttatgtcaaatttaactacaggaaagggcaaggcattttctaagt
tcettagatgtcatgtggctaaaaaaaacaaaaggatggacageagttagatactgtacacttagctgtttgaa
gccatatattcagaaagcagatgttgggagttggtgtttgaggactgatttcctggaggtattttatataggcc
aagttcattgttctaaactctaagggcttgacttgagggaggaaaagaggcaagaacatgtttagttttgctga
cagcatcacatgggcagccctaaggctagacaactttagggcctgaagcttattctaggaaagaagcacctaca
gagtggcactgggctcccctccactatagagatgaagtcatatgacagtaaagggcaggcagggctgcctaggg
ggcccagaactgacacttccattagaatgagcacaggccagggagagaagtggggaaccagagagaaggagctg
gaattctagtaggacaaacggtaagtgaacaacaagaacaagttaagagtgtgtgcagtattctttcaaagact
gaaaaaatagtgatgtgatagaatggcaggtggctctgagcaggccaggagaaggactgggggcagagcatccc
aggcaggagggcagcaagtgggaaggccctggggtggggcttttggactgttccagtgacgggcaggcagccag
tgtgcctgtcacacaatgcaccagggaagtagtcgtgaatttgcagagggtcttgcaggctatgggaaagggat
tggattgtattttgtttgtagggaagccatcgggggacttaagcagaggaaggattggcttcatctctttgaaa
aagttctctctggatgetgatgggaggagaaatggaaggaaaagaaacacttttaggggcaagaacttttgaga
agggtggaattgggagtgtggagttggggccagctttggcacaggaggggaagctaaacacgtggccgcatgag
ggcctgtaattctacctgaaatgggtaccatttgttagggtaaacaaatgaaccaaatgcccagtgatacagac
caagtgttggcaaacttcttctgtgatggcccaggtagtaaatgtctcaggcttcgcaggccatgtggtctctg
ttgaagctctgtgtagtagacaatatgttaatgactgggcgtgactgtgtgctaataaaagtttatttacaaaa
acageecgtgggctggatttageteacaggctgtagtttgccaacctctgacctagagcatgaactgagcatct
tcttggagggaaataagttctttccaagttgccctcctcacattgcagggggccatgtaggcccattattcaca
gaagagtgggtgggcaacctttctggagcagaaaaacgtaaagatttcttccgtagtgcaagtaaggtgaccat
ttctaaaccgtgcaagtgatccagcagtcccaaaagttgtttcacttctcattgtgcgcccgttctcaggtgct
ccgaagcttccagtcctttgtagggacatggatgaaattggaaatcatcattctcagtaaactatcgcaagaac
aaaaaaccaaacaccgcatattctcactcataggtgggaattgaacaatgagatcacatggacacgggaagggg
aacatcacattctggggactgttgtggggtggggggaggggggagggatagcattgggagatatacctaatgct
agatgacaagttagtgggtgcagcgcaccagtgtggcacatgtatacatatgtaactaacctgcacaatgtgca
catgtaccctaaaacttaaagtataataataaaagaaaaaaaaaaagagaggagagaaacatcatcccctccag
gatacccttgggccttgttcttatagtcttgtacattgttgaacaatttgcatgggctagtggattaaagcaca
ccctccaccctcaggccctcaagggtctctatgataatacagtctcaccttctaccctttccatcaccatccta
ggtgctatggccaaccttgaggctgccatgttaggtctatgcatttcccacctccaccacataactctctgaag
gccaggtagtttcctattcatcttggtaaccccaaagcctcgtgacagggctcagctggcatctgcggatgtga
atgaaccattggagaaaatggtactctgcaaataactctgttattttcccatttcctgtgtaaggcctagagac
aatgactttttaattgcaccccttcccctctgtatgacactggccttctcttgtgtccagcaatgtgggtggcc
tagatgatttctaagggacttctggccaagatgaacagcagctgcatcttactgagcatttactatgtgccata
tactcagecacagetctaggggcatagaagcaggagctctcagggtcagggcagtgagtgageaagcgageace
tatgeeagecctgcctctggatggggacttgagagggtgatggaagcctgcagcactggagggaggcagacaaa
gacaggcctgtgetgagggggcceggageaagagagagggaggcaatgacagcagagacatgcetgcgccttgg
gtttgagtgcccagtggtcaaatccacttccctgtggctgatgcttgcctttctaactttggaatttaggggtt
ggagatctggtgagaaggtaggagggagatgaggaggagaagggaaaggcaggaaggaaggggagggaaaggaa
aagcaaaaggggaggaggaaggtttccaacaaattattctatatcaactgcggaaatcaaaatttgttgcccaa
atcttagaagctcatgtccctcctccccagaagtctggaatgcagcactccaggggtagcttataacccaaata
tetatctgtaaaaagagaaacattgggctttcgagctgtggattctcagtaaaagcaagaggcctcagcctaca
caggccagcccagagtttgaggaaccccaggcccacacccacagggctggcccctgggtctgcatactccctag
aaatgtgcacacttctgagcctcaactctgtcctggagtctaacagcatccctctccttcctggggcagttcca
cctccagaaacctgttaccttgggccttatgtcaaggaaactgtgggaaagagctaggcaggaatgcagatgag
gccagcatgggctcctaaaagtttagaaataggcagtgtcatgetcccaggtgcctgcataaaccagctgaaaa
atggagctcccctcaccagcactctcccttcaaacagactgtgatttgcaggtcactggtttaccaagccaggc
tacccaggcaggacccagatgccaagcccagtggtgtcctgcaagctgagcagtgctcagttcttgcaaaaaaa
ggtctgtgtgaaggcaaggcctctgcctggcttctcaccccagttgggtgtctggaacaggaaggagcccttac
tgcagaaaaaggaggagggagcaaagggagcgaacagctgcgtgctccatggggaggatccccaaagtagaaag
gcgcatacacactgcagcccttgacccagaatgctcacagctacattacagattcaggtctcctcagtgtagtg
gggctgetgatgagactgtggcatcctcaggggtcaggacacacattttccatcactcttetgatggcaaaaaa
cctctgagccaatgccaacctctgatcattaaaaaaaagtgctcacagcagtgtgtggtttaggatcatgccct
gtgtggtttggaacacgtgcacaaccacaccttgttcatcaccatcccagaaaccctgacgcaggcaaagagca
gagttattaaccctactttactgatgtggatactgaggcccagaggctcatgcaagttatcaataagtggcagg
gacagttgcctctagattaactagcccctaggatcacctgggtcttggaaggggacccataaacatgagctccc
ctctcttggggccagatttgcacctgtgccgcgccttcagectgcatgaagtaggggctgetggcaaagactea
aagctgtaaatctgggttttctcttgaggcttctaagggagctgtttcgacaactcactctgttcccagctggc
tgcccctgcatagggttttaaagcagcctagctttctgccaggcttggcagtggacaacgctggtcagaacatc
ccagagagctaccagaatgaagtaagtttgcttctactctttacctgtttatgggctgtctctgccactggaat
gaaaggcactgagaacagtgcctggcctgcagaaggccctggaaatacctgagctcctaatctgggaataggag
taggaagagctttggaggcagggcacctgagtttgagatctacaacttcctgcctgtgtgacattgggaaagtc
tccatcctttctgagcctcagtctccaccctggggaagtggaaatatcaatctctgtgacacagaagcaaatga
gcgaatgtgcacaaagtaccttgcacaagagagacgctcaaacacttgcctccaggtttcaccgagaactacag
agtaagatagatttgttcccagtggaggaagcctgggaataatttgcccctagactatgaattcctggggctca
agatcgagcacagggccaggcacacagaagggaccctggaaatgtggcaggaggccagagatagacaggccctt
agagctcatacccatgccctctgacctcaagaagaaagaaacctgctcaaaatctcacaaagagcttgttccaa
ccctgaatcgagtctgaggactccttcctgagtccagcactttttctgcaagaagtatatgcctccaaagctga
tgggcgcaaatcttgaaccccgtcacataaacacaaagggaggaggtgactagagctcctcctactggatatgt
ctaaggtcaccagtctaaagaaaagggatggatagaatgaggccagtatttttgcagccatccaaatgtccaca
tacgctgttacactgagggctcctctctcccccgtcttcagccctacttgcatttagaggtgagaaagatatgg
gctgaggggttgtttttcatcgtattgtagatggaaagcacactgcccttggggccatccaaatgtggaccttg
atgtagcaccccaccttctggatggccatccttctgaaagtcactgaatttctcagactttattctctttatcc
ataaagaaggagaataataataatccccccaccctgcccaaccactgactggttgggaagctcagaagaaatac
tgggcacggcatcccattgtaatctatagagtgagtcgcttcttaatattaaatggctgaacacagaagatgtg
caaaaagtactgtgtccccttcctcctccaactgaacatttcatgccctttgcaccctcattttgtctaggagc
tgccttatgaagggaataggtacctgctccgagctggaggaatctttgccacttatggtggggtatggactgag
acagagatggcatgtgacatgcgcactgagtctcaactccatgcaggctctggagcactctcaaattggagtac
taatgccttttaaattctcacactagcaatcctttgacctactgatctagggatctagggaaagaatcgtgate
ttaacttcaaagggaaggacaaaatgttctgcctcctgttaaaactccatacactaagtgcagagactggatgc
cttattaaccttgggtagatgcccaaatgttcaaaaggtcaaactcttctgttccccagatcgccagagtcatt
aaccagtcacactattaaatgaatgaacagatgctgaaaaggtacttgcattactgagatttcttatggtgatg
gcccctgcctgatatgtattcagcattttgtagttttcaatgtgcattagagtatagtggtgatgacattggcc
tctgagtttgccacttcttatatctgtgactttggtcaaattgcttaatctctctgagtctcggtttcctggag
ataataatagcttcttcttcccagggttatcatgaggattacaggagataatgccccaaaaatgcttagtaaag
tgcctagcacctagtcaatgctgaattaaaggtggttattcttacttttcgttcatttgaactttgttctcagg
gagggcaaaggatagacaaagccccatagctagtgaggagtagctgcaagactagaacccaggtgttctgagcc
ctagtcttaggccaagaacaactgttacgtgagatgcacgttttccttcaagggagctcacaattatttccatg
taaattcaaggactgctaaaagagaactctcctctgggactgatatcattttatttcaagattgatttgaaaca
tgttttttgtttgtttgtttgttttctaggaaagaacaagagaaccagttaagctgaatgcctgaagcaaatcc
ctgttagcgatgttttcaggatgagggagagtggtgcaagaaacgtgcttccagatgcacatggtttcctggga
ctagggttcagggtgtcatccctgggtgttattaagtgtcagaaggagagcaaacaagggaaacatctgagatc
cagctaaggctacaccctggaaatgcaagcccagctcttgcaaaggacctcctttggccactcaccttccaggc
cttacaataacttgtttggactgcaggtttcttggtggactcacaggccattctgcttttatttggtcaacctc
agttcacaagcacccagatgctgagatcctcagcatgtgcagcagagtttcatattagcactgggtacctttct
gaggctacagggataccgtacagcagcacctgtcacgtccagccaaaggagtgggctctctcaatgtcatccaa
tgctgtttcaactgtgaagaagaccatctgagagagttgcttttggaggctgaggcaaatttttaaaattcttt
gttctcctcaactggggtgaattcttggtcttctaggacagcttgaagttttagaaagagtcaagccactcaga
accaacagagaactctttcagagaacaaggtgtggcatagaggaggcagagggctgatcttgatcaaatccaaa
gtgtgactctaaagcaatgaatgtgaatttttggcaaagcttacaaagggctctaaaggccatctgcaaagaga
agccaagcctgatcgatgaatcactagtgcggccggatatcgatcggcacgctgttgattttctcatagtaagg
aacagtgggccctttcagtcccacttctgtagtctgtggtactacaaatggtgagcccatgatgttgccattca
tagggttattctccagcagtaatgactggccagccactcccatagccgcggggctaggatttattgtcaatgga
gggacctgcagttctgcacaagcagtactaggatgagcacctgggcccattgcaagggtgacatcttcaaggca
aggcctcttaattttattagggtagcccccatcagecatgtctggaaactggaagtggtcttcttcttgtctcc
tcttaacagttccctgtgaatggaagagaagagaggaggagaagagaggagaggagaagggaagagaggtgaca
cacacacacacacacacacacacacacacagagagagagagagagagacagagagaaagagagagagagagaga
gaggaatttttataaaggtttggcacattaaagctaatgaacaggaaatgtgcatgataaaacagacctctcag
tttaaagacttatagttgtgaaaactataaaatacagcctgtctttggaaccatagtgcttatttattcattat
tatgtttcatctaaactgtctaattacatttcaaataaggcattatgttgtctgtatactaaaacgggatagaa
cgttattcaaagggtaatctgcccacttcaaggagagttcaacaaaactatgcagaagtcactaaatgaaccat
gctgccaaaggcaggcattggagagaaaactagaagtagctaaatagttttaattctttcctgtctacagacac
atagattttaacgaaggaataccatagtatagaattgaacttttaggctgccttctagtcttggttaaatgcat
caggctgcagtggtaaaattgaatacaacagagcccttacaggaaagaagtagatctggatgtgttttcttggg
gagctgtttaaaatactgtttttgggaaagcacaagtttcagaacagtcattgtaggcatcgtattcattgttc
catttatttttacacacacacacacacacacacacacacacactctcacacattgctatgtgtacacaaaaata
atttggaagaacctatacccaacaatttggagtggtcatttatttgggatgactggcaattccctttctattct
cttcatttctgcttgtttgtctttaacgagaacgactcataatccaaaaatttaaaaaagtataaagttatcta
aataagaaattttcctctgaagatgcatcctcaggttggggagatattaaacaatgagaaaaggccccaatctg
ggatctgaaccttgggggagctgcccatcatttatagaagcacagcctttgggaacaaagcaaagtcactagca
atgtgagacttcctactcttcatggcttcatacagtcatccatcgctgttgtgttaatgaccatgacctgtatg
ttagcaggtaaatgggaaaggaagtgggggcaaaggagtatgtgcaggaatgatcaaaataaggaaaggaagag
agggatctggaaatcacctgaatgccgataggtgaacaggtagaattcttttaaagcttcccccacccggtacc
ccccaaataacccctttccagctttggaagtttcactaggacatacagtgetcatoctetgatgtcacettaag
tttggctcttctggtttgatgagcttgtagcccactaggagctcaaggcatgcatggggccacttgccagcacg
atgaggggcatgactgtcatggccaagtgaacatcaaagcagatccccagggctgtatgtctcaggccttggtg
cacatcagaatcacttagaaacatccacattcctgggccctcccaccacaaactgacagcttcatccagggtgt
ggcccaggcatcgggagtttttccaacagctccatggctgattctcaacagaaaaccactggcccagagcaagg
gtggaggcagcgtggcatagggctctgaccttggccttgccactgaacctctcagagccccagtttctttatgt
gtaaaatgagtgtaattatagttcttttctcatgaaggtgctctgactattaagtgaaacggggcacattgtat
gacacctaatagctcctcactaactggtacccggcattataaagggcaggtatggaagggttctgggagtccaa
tacccttcttaaagacagagaggtctctgagacccagagaggggcaggccttacccagagttgctcagccagag
ggcaacaaggcccaggtcagatgcagggcccctccaccaccactcagctgcctccagacccactgccttcgcca
tgttgttggtaggacactgcatcgcccccacagaaggggcttgccaacttgagtgagaggacttgcacacttct
ttgacttttcttttgagatgcccacaatctgaacaagggcacttcaagggacagctctgtcaccaaactcatct
gaggcctgaataccatgggtcaggcaggaatgggttggagaggtgtagagcaggcacaataagagggctgaggc
ccatgcagtcatcagtgcccactttcccaggagtctgactgggcacagcacccatagtgtccctgagctggtcc
atggagcagctcactaactgtttggcccacagcaggtgctcagtaaatggcagttgaacgaatcaatggacaaa
ggaacataaattacccaacacacagggagctcagccatttactcaatccattatggagtaacctacaaacaagc
cactgggtcccaaactgaaattgtgtctcttctacattctcccaaagaatccaataggttaaaaatagaaatgt
atgaaatagatcaatcagggatgattgcatgtggatttgacataaggatcccctgcagggagtctgagctggca
acagtcaggcccaaagtgctgtccatgatgtctcgaactgcaagacagttttaacaatggcgaagcaatgcaga
accaggcaggccaaggagggggtgggggttggggaaaggaagggagggaaggggctgtgaggggcaatggtctg
gcatccctgccacgtgagcctctgaaatttgctggcagcttctatgggctcccagagctttcacttaattgttg
gtctgccactaacctgctgggagtaaggtgcagggatggaggaggcagggcatgaccaccagacactaaaggta
ccagctggggccactggcaaagggaaggaggctgcacctctcctacatgagagcccgtatacacacaccttttc
cagcactcatcaactgcatcccaagcaaatggtccctgatcaattccaattctagaaaccaactgactactcaa
taacaaagtagatcccagcaggccgccactgctggagcggatgccacttttgctatgccaagtctgtggctgga
cagctgctggcatgtacactcactgactttcataaggatgcctaataaagggggcaggctcacctggcttttct
caggggtggggtttggggtgccgatagaggctgctgttttggcagagtggcaagctgcaagcctcttctgagct
ttcatttttcaatggacttcagtgagaattcactttgtcagaggccatgcagctccatgttttggatttcatgg
aatgagetttcaacagtgagectgaagtgccctggctgaacageaagaacaccagecaaccctaaacaaggceg
aggagaggcggctgtgtttacacggaaggctcagccttgctgtaatagcgtctgccttcaccagacatcagtga
ggcgtggaaatctattatccagttaattttgcccctagataaagacttgctttcgtgtcttctctttcacagtc
ccatgatctgttactcatctcaactgcgagaagttggctgggctttcccctgtgcccagtgccacactcgtgcc
ttcactgggtcacctgtgcctgtggctgatgccgctgaggttttgcctgcccagactgggtgtttctgactaaa
tcccacagccaccattttagatcaagggcaggagatagctcactgctccggaatgacctcccctcccagaatcc
tggtaggggcggaaggtccccaaccaagctcccagccctttctaaatgaatctccctgcttcacccatgtgctt
ttctccagtctctgcggtcttgatgacagcagggtattagtcctagctgtcccacagctcctacttctttcagg
cctctccctgtgacaatcagtagccactggcaggatttcctcagagcatatctcgatttgctttcagacaatta
gttaaaaggacactggaccccagacgtcccaactcccagccagagccctcacaggcccggcctttggtggtgag
gaagggggagggagtgagtgacagtgccctggcatcttttagaaacgaattcctttctctccatacataaatgc
ctgcagagtcccatttcagaatccggcagacaaagccaccaatgtgatccccatgaccttataaacattcatta
aaatgcatttcaaggcatgtgatggcctccccaccccctagataatgagaaaacaaaggtttctcttctgatag
agacaagttcagctctgaagtcaacattatttctggttctgtctgaacaatgacatatggcaactcttcccttt
ctatagttctagtccagaatgacaaaaaaggggaaaaatttcttagagaaggtagagattatacgaatacagtc
catgaaatgagcataaggagaataaagaatataacttatccaaagaagtctggcaggctgttataaatgcttga
ttttggacactgtagttggaggtttaacatggacaccaataaaaaggtcagcaaagggtatgcactgttcctat
tgggcaagaagataggaggtcaaaggtaaccaggaaagataaactcagggagacttattttccctccagagggc
actgggcttgtaggccctgggcaaaattgtcaaaaaggtgaaaatcgcctgtggtttatttagtctgctctttc
ttcactagtgcctcaccagttcagttcaggccaatttgctagaaggtagcgaacgatcgaccggtgaagttcct
atactttctagagaataggaacttcggaataggaacttctacetagatgcatgetcagagcggcccctagetag
cgtttaaaacctacagttgaagtcggaagtttacatacacttaagttggagtcattaaaactcgtttttcaact
actccacaaatttcttgttaacaaacaatagttttggcaagtcagttaggacatctactttgtgcatgacacaa
gtcatttttccaacaattgtttacagacagattatttcacttataattcactgtatcacaattccagtgggtca
gaagtgtacatacacgcgcttgactgtgcctttaagcttttaattaagagtaattcatacaaaaggactcgccc
ctgccttggggaatcccagggaccgtcgttaaactcccactaacgtagaacccagagatcgctgcgttcccgcc
ccctcaccegeeegetctegtcateactgaggtggagaagagcatgcgtgaggctccggtgcccgtcagtgggc
agagcgcacatcgcccacagtccccgagaagttggggggaggggtcggcaattgaaccggtgcctagagaaggt
ggcgcggggtaaactgggaaagtgatgtcgtgtactggctccgcctttttcccgagggtgggggagaaccgtat
ataagtgcagtagtcgccgtgaacgttctttttcgcaacgggtttgccgccagaacacaggtaagtgccgtgtg
tggttcccgcgggcctggcctctttacgggttatggcccttgcgtgccttgaattacttccacgcccctggctg
cagtacgtgattcttgatcccgagcttcgggttggaagtgggtgggagagttcgaggccttgcgcttaaggagc
cccttcgcctcgtgcttgagttgaggcctggcctgggcgctggggccgccgcgtgcgaatctggtggcaccttc
gcgcctgtctegetgctttcgataagtctctagecatttaaaatttttgatgacctgetgcgacgctttttttc
tggcaagatagtcttgtaaatgcgggccaagatctgcacactggtatttcggtttttggggccgcgggcggcga
cggggcccgtgcgtcccagcgcacatgttcggcgaggcggggcctgcgagcgcggccaccgagaatcggacggg
ggtagtctcaagctcgccggcctgctctggtgcctggcctcgcgccgccgtgtatcgccccgccctgggcggca
aggctggcccggtcggcaccagttgcgtgagcggaaagatggccgcttcccggccctgctgcagggagctcaaa
atggaggacgcggcgctcgggagagcgggcgggtgagtcacccacacaaaggaaaagggcctttccgtcctcag
ccgtcgcttcatgtgactccacggagtaccgggcgccgtccaggcacctcgattagttctcgagcttttggagt
acgtcgtctttaggttggggggaggggttttatgcgatggagtttccccacactgagtgggtggagactgaagt
taggccagcttggcacttgatgtaattctccttggaatttgccctttttgagtttggatcttggttcattctca
agcctcagacagtggttcaaagtttttttcttccatttcaggtgtcgtgagaattcgatatcccaccatggaca
aggattgtgaaatgaaacgcaccacactggacagccctttggggaagctggagctgtctggttgtgagcagggt
ctgcacgaaataaagctcctgggcaaggggacgtctgcagctgatgccgtggaggtcccagcccccgctgcggt
tctcggaggtccggagcccctgatgcagtgcacagcctggctgaatgcctatttccaccagcccgaggctatcg
aagagttccccgtgccagcgcttcaccatcccgttttccagcaagagtcgttcacgcgtcaggtgttatggaag
ctgcttaaggttgtgaaattcggagaagtgatttcttaccagcaattggccgccctggccggcaaccccaaagc
cgcgcgagcagtgggaggcgccatgagaggcaatcctgtcaagatcctcatcccgtgccacagagtggtctgca
gcagcggagccgtgggcaactactccggagggctagccgtgaaggaatggcttctggcccatgaaggccaccgg
ttggggaagccaggcttgggagggagctcaggtctggcaggggcctggctcaagggagcgggagctacctcggg
ctccccgcctgctggccgaaacctcgaggtgaaacagactttgaattttgaccttctcaagttggcgggagacg
tggagtccaacccagggcccatggtgagcaagggcgaggaggataacatggccatcatcaaggagttcatgcgc
ttcaaggtgcacatggagggctccgtgaacggccacgagttcgagatcgagggcgagggcgagggccgccccta
cgagggcacccagaccgccaagctgaaggtgaccaagggtggccccctgcccttcgcctgggacatcctgtccc
ctcagttcatgtacggctccaaggcctacgtgaagcaccccgccgacatccccgactacttgaagctgtccttc
cccgagggcttcaagtgggagcgcgtgatgaacttcgaggacggcggcgtggtgaccgtgacccaggactcctc
cctgcaggacggcgagttcatctacaaggtgaagctgcgcggcaccaacttcccctccgacggccccgtaatgc
agaagaagaccatgggctgggaggcctcctccgagcggatgtaccccgaggacggcgccctgaagggcgagatc
aagcagaggctgaagctgaaggacggcggccactacgacgctgaggtcaagaccacctacaaggccaagaagcc
cgtgcagctgcccggcgcctacaacgtcaacatcaagttggacatcacctcccacaacgaggactacaccatcg
tggaacagtacgaacgcgccgagggccgccactccaccggcggcatggacgagctgtacaagtaggcggccgct
cttcgagcagatatcataagatacattgatgagtttggacaaaccacaactagaatgcagtgaaaaaaatgctt
tatttgtgaaatttgtgatgctattgctttatttgtaaccattataagctgcaataaacaagttacgttaacaa
caacaattgcattcattttatgtttcaggttcagggggaggtgtgggaggttttttaaagcaagtaaaacctct
acaaatgtggtattaattaaagatctttaaacaatttaaaggcaatgctaccaaatactaagcgcgtgtatgta
cacttctgacccactgggaatgtgatgaaagaaataaaagctgaaatgaatcattctctctactattattctga
tatttcacattcttaaaataaagtggtgatcctaactgaccttaagacagggaatctttactcggattaaatgt
caggaattgtgaaaaagtgagtttaaatgtatttggctaaggtgtatgtaaacttccgacttcaactgtagttt
aaaacgggcccgtagtctagggccgccagtgtgatggagttcggcttcaggtacagcacactggcggccgttac
taggtagctagagccttcagactctagggaagttcctatactttctagagaataggaacttcggaataggaact
tcacccatggcgatcgctagcctctaactcctagaccgtcagaactgctgggcccttcaagacgggctgctcac
acccactcatgttaagcctggtgaggcctgtactctgttttcacaggaagaaatcctcacccagtcttccccaa
acacattcccaggttgtgtcattagtgggatagagatgattattgtggggagaagagaaacatctggatggatt
tggtgaggttgatctatagaggaagtaggtgctgcctgaggtagctgtaatagaagctaaaggtcaaaggagag
ggccctgtcccaatccagatgactccacttctgctggacccaggttcacaagcttaatctacatttcacctaaa
tttggctaacaagcccaaaatcacacaggcaaagggagaagtggaggcagaaccgaggttggaggccaccaggg
ccaccgggcagagatcatttaagcccaaccttctcacttctccctgggctctgcctctcttaaaggaccttgtg
gtgtgacctcttgtaggtccctttcacactcggggcctcagtttccccactgtaaagtgaatgggtcccagctt
tggtaagcttatgcttacctgatgctttcttcctgggctgctcttgtagagaaaagataaatcttcttcctcca
tccacgagggctcctttccctgggggtgagagtaggctgaggagagccacttgcacacacccttaaagaaagta
ttacctgcaccagctcagtgagaggcacagatcagactgttacttgaatcaaattatgagcctccccaaatata
tctatgacatttaaataggggattacttgaacatagactgtgggatccggtgtggagtgcgggagactagcaaa
gtgaatcctgagagtagcaggtctgcacctgttggatcgagaaaggcggcctacaattctggtcaaatgagctg
tgcttattgacatattctattagagagtactaccaggtcaccagtcaccagaaaggctgccagctctccaacca
cctccagggaactatcctgaatggggccttaacaagtctaagagagggttggtttgggtcccaagccaatattt
gctctgctttatgtcagtcatatggaacccaaaccaaccctctcctatgtgcctcaccagtcggtgcagggatc
ccaatttcaagtttggttttttatggtcaaagtccagcatagattaaatgaaggggtgtgatgatggtgttaaa
agagaactccagaccagtttaactcttggacacacatcccatctcaccatggtgcttccaaccttccagagatg
atgggctcctattttctgatgacaaagccctccacaggattgctgcctggccatcagggagtgcctctgtaact
gaggctgagatcccactttcagtcctccagctgtggcccatccctgctccgcccaccgggtatggcctgtccta
ggctcttaggtatggctgcattgtgaaatgatggctacagagctggcatctcctgtagtctggttcatctagtg
cactacctcatagttaaaagaaatctgtttaagccactgagggtggctcctagtgccaactccaagaacaggaa
gcttcccttttttgggaggaggggcagatggtaacatggatcgtccaggtcaatgggagcagggcaaccacagt
aagtactggacaacaacacaaaactccatgtgtggcttccatcgagtccctctccaattggtttggtcttctcc
gtcccatgcagcactttagcaaggggcctggctgaaggctatgaattgtgtggagcctcctcattgcagtctcc
aaccatctgatgctgggaaaatgtcaccaggatgcagccatgccgtgtggccaatgaaccgagaaaacacccct
tttctagaatgctctaaagaggcagaataatccagaggtgaggaaggaaatactccaccagagacccaggcagt
tcctacaaaagccagactttccttcacctagggagtgacaagaccagtggaaaacactctcaagcagtaacccc
caaatgctctgcaagccagtggcgtccagataccgcacaagcgagtgggctgtctaatcccatcatcatgatgt
aaatatctctaggctgccctgggctgtgcctgaccctgtcttcagctttccacacctccacctacagcccatgc
acagaaggaccacccaggaatgctgcaagtgtggcacctccagggccacccagggagaaggagggcagctatgc
tggtggctccaggcccatttggcgggtggtaccttcacaccacaaagcccaaactgaggccccagatttggctg
atgagggcatattggacaggggtcacttatgctcttccccattgccacctggcctctggctacctggacttggc
tacctgtggatcctctcacaggtgccaccatcttggctgagtctccagatgcgaggtccctgaggcagtggcgg
gcttctcgctaatgetgatgggattaggaatgggataggtggggagggccctggactgggccctgatgagecaa
gtgggtttttagaggggctactggtacatttcagggacaggacatctggtagagctaagctggggcaataagga
gccactgctaatctgagagctagaaacaatcagcttctgggtcattattaattagggtagtttgggctgtgtgg
aagtcacgtactatatggggtagccacagctctctctacagataatctctaagacttctgattgggactgtgtg
aatgcagtagcaatatctcttcttactgccaggccctgccagtcctgcctccacgccctggctggcccccctta
tgatctgacccatgccaggctgccatagtatgttacttctgcattagcactccttgggacctgcctctccactg
tccctcagactttaaagaactatacaaacccaaggggctcttcccaagagaattgatatgacttgaggtgattc
catttctggaagtagtcactccattttctgcctcactctttcagtgcttcacagagcaggttcgaacgaaggag
ccatccaactaaccgtcatgttcgggcaaccgaagaagggagtggcaggatttcctttggagacttctggaatt
agacagcagtttaatgcaagcatctaaattctctccctcccagagtctcattaaaactacagtaagagtttgtg
ttttgttttgtttttaaagacaaaatcccaccaggatagagagaataggagaggagataacagcatcataattt
atgaaactaaaatgcagatagaccaggattaactgactacacagcaccaaggaagctgaatcacaagacagcag
aggagaaaactggaaaggatcgtggtctatacggcagaatcttcccaagcctcaggaggaggagctctagatgt
tcccagatctgggaggtaaagtggaatggggggacatggtcagcgtaatggggttgggctggaagcaggttaag
gagcaggcagatctctgaatcccctctctgactctgtgtccccaggcatctgcctgtcccccaccctggaagag
gtctggcttgaccctttgtctggtgaatttcctgctctgctttcctggtcctgctggccggatcagtggaggcc
actcacttcaccccacagggatgttctgtgttgccctacacctgggaactggaggtactggaggcaggctgtgg
tgagcttgaaagcaaaacacagagggcagtccaatctctttggccatatttcttctgcatatccaataccatgt
ccacaactctgctagtgtcctgatggtggtgggctctacacattcccgggaagctgaaggcagataatgaccag
gacaggtcaacctctcttcttctgaaagccttcatctactaatggcctgggactcttcccttaaatgcttagat
tgtgtcttccactaaggttttttgctgttgctgttgtttgtttgtttgtttgtttgtttgtttgtttgttttga
gacggaatctcactctgtcgcccaggctggagtgtagtggcacaatctcagctcaccacaaccttcacctccta
ggttgaaggggttctcctgcctcagcctcctgtgtagctaggattacaggcacatgccaccatgcctggctaat
ttttgtatttttggtagagacaggatttcgccatgttggccaggctggtcttgaactcatgacctcaggtgate
tgcctaccttggtctcccaaagtgetgggattacaggtgtgagecaccacacccggccaaggtttttgtttgtt
tgtttgtttgtttgtttgttttgtattgaggcagggtatcactctggtcacccaggctggagtgcagtagtgca
atcacggctcactgaaacctccacctccctggcgggctcaggtgatcctgccacctcagcttcccaggtagctg
ggactacaggcttgtaccaccactcccagctaatttttgcgtttttagtagagacagggtttccccatgttgcc
caggttggtctcaaactctgggctcaagcgatctgcctgcctcagcctcccaaagtgetgggattacaggtgta
agccaccgtacccggccccgccactaaggttttgaaaatgaagcaattacaagtttaagtctattaataagtga
tgaagccatgtagaaaagcagaataattatcttggatcaggaaggtcacatgaggatctacttgggggttgtca
atattctatttcttgacctgatcagtgttgacagcaggttttaattttttacttctttttgtttgtttgttttt
gagacggagtcttgctctgtctcccaggctggagtgcagtggtatgatctcggctcactgcaacctccgcctcc
tgggttcaagctgttctcctgcctcagcctccccagtagctgggattacaggcaggcaccaccacgaccagcta
atttttgtatttttagtagagactgggtttcaccatcttggccaggctggtctcgaacttctgatctcgtgatc
cgccctccttggcctcccaaagtgetgggattacaggcttgagecagcgtgcccggcccattttttacttcctt
attaaactgtacatataggccttgcacacttttctgcatcaatgttatattccacaataaagggaaaaggtata
tacacaacttgataccagtaatgtgaaacatatatttctacatagaaaaaaaaatgactgaaatactgcactcc
aatgtgttcacacagtagttgtttctggattatttatatattaaatgtttatatattgtattatgccatgaggt
ttgtgttttctctccacttttctgcattttccaagtttactacaaagagcacatattactcttataatcagaaa
gtcataaaatatatttaaaaagacaaaattgaaactaataaggatcaacacaaaacagatgagccatctgtgga
aatccgcacagaatactacctaaagagattggtgacgtgcatgatctcactaggatgagcacaaagcttgccag
agcctagggtctatttctagggttggctcttggaagccaggatagttgttatctctgggaagagggaggggcac
acaaggggcttctaaaacattctgaatgttctatttctgaacctggttggtgggtacatgactgttggttttat
tattatatgttttatatactcttccgtatgtatggtgtggattccaaaaaaagatttcctttagagaaaaccag
aatcacataagtagaaaatatggtgctatgttgaaggaacaactcaagtttatataaaatcatcatcatttata
ggcttaaaaagttgctttggaattttggtctaactgacttgtcttttctgcagcaaaccacgctccttctggac
gtgetccaggcagaggggattagggtgggttcaaggctgcaagtacetagetcageacactctettcaggggac
ttagagtttgtctggtgttggctctctgagctcttgtcaggaatgccgacccttccgaggttcaggatttgaag
cctgccttcccaccccagatttggtccacacagacactcaagtatgtatttcaactacaaatgacctgtacttt
cctattactcctctctttcatggtaacctttctggtatccttccttccctacatttatgggagggggacatcat
tctctgctctcctgtcactgaaggctccaccttctgtcttcttctgacccatctggttttcctggggccacctc
ctctccttaccaccctaacgcttttgtaacttgaggagaaatgagagatcacctagtcaggtcatcattctctg
tagatgaagaggcccaatggtttgctcaagaattgccaagcgagttaaagacagagagtatgagagtcagcaag
acctacagaaagcatctatctgcactgttttgcagggacttagcctttgtgtgtggactcctggaatgccaccc
actaagaaacattgtctgacaccaactccccacttggtaggtggggacactgaaactcatggcaggaaagggcc
ttgccccaagccagggcagagtgtcactcatcactctcaattttcagtccagggcaccttgttgtgactatccc
aaaggcagccactttccctggtctgaaagacctgaagagagaagagaagagaaggatggaaggcagagtatgcg
gctttgattcatttcctggtgaaaacagatctatacgagaagcaaatttcacgaaagggaagagaagaaagtgt
cccatacgttgctggcctgtttcaaccttgctttgattcttgctgaaaagggtaccgtgtatttctgagttcaa
catgcagaccagtgttaggaaagccactgcacctccactttagcctccagggctgtgccctgcaaatggcctgc
agccttggtgcctcgctctccagactgcattttggaagatgggacagaggcttatggaagcccacattagaacg
ggggagcagaatgggtgagatgagggatccttgatagtgaaccagatgaaggaatggtagccaaatgccaggcc
tcctttgtggcttcaatccaaaggctctggagcccttccagggcagaacatcaggcatgtttacccccactgtc
ctcaacagtgacagaggtgcaatcttgggcagctggccattttgaaagcaacctccttaatctcaactgggaag
gctccctagcaggacccctgtgttgcacacctggaggaagctagactaaccagaagctcagcacggttccatct
gggatgcccaggtctgagacgaaaaaggtaactctcttttctgggtcctggcccagttgtgtctctctccacct
cattctctgagatgcctgtctccccttttttgtcccatcaggaggcaagagctatcactgggccagactccacc
agaagccaagccagcttgttacccagcttctcagggagcaaagaacagccttgtttctatcttatccccactgt
cccctgcccctgccccacctcccagccattcagcttctggcttccccagagctgcctgcttctttgtggtcctc
cattccttgaaaagaccttctagtcattagtgtatataaatggccacttagcccagattacagtgaggtcaaca
gctggggctctgagaattgtcacacactggcacaggagaggaggctattcttccagagaatttggagggcactc
ccatccacttacaacaaaaagcccatccactgtgcttggcagtaggtgatctgagaaccaatggaaccaggtta
atcctgtggcactgttgagtgaggagagcagtggcgggcactggaaaatatcagagacaaggcaggagacctga
aatctaggcttagctcctcatatacttggcagctgtatgacctcagacaaccagtgttacctctctaagcctca
gtttcctcatgcaaaaggagggggaataacaacagagcccactgcttgggggtgttgtgaggacaggatgaaaa
aacaaacagaaatccctcagtacaggattcagtgcagtggacagtcttgcaaggtctggttcagccctccaccc
ctaccctcaccagtataaagaactctggcctacaagtcagatgacctgagttttaatctcagctttgccattag
ccgtgtgaacttgagaaagtccctttcctttttacatctattgggatgatcatgcattttttgtcctttattct
gttaatatagtgtgttacattgattgcttttcatagactgaaccagccttgtattccagggataaatctcactt
ggtcatggtgtataatcctttatacaaatgttgctgggttgagtttgctagtattttgttgaagatttttatgt
cttgattcataaggaatattggtgtaccttccccttttatggccacagtttccctacaatgatgtagtcgaact
agacaacctccaatatctttcagtattcatgtcctctgattctgtgaaactaagaaaattaagaaatagtgatt
cataggcacaaggcaggcaaaacttagactccttgtagaataattaggaagccaaatattcagtgtgcttattt
ctcaaataaccttagtttctccagtctgccccaactccgaggcctgaatatctctagatgcttatgatggcaac
taaagcctaaaagctaattcattttaaagttcttccaaatgcatagggttttatttttccagacctgggttcag
atggggaatttgacaaacaatggaaagggggaaaaacaacaatctaaacactgagtgacaaagtaacaaagaaa
tagtctagctatcagccagtcaagccagccttggctttgctatccaaagtagtcagtctaattctaccaccagt
ttctgttcctgtagctgtctactgcctgccagggactctgccttcccacccacaactaccaatggaaggatgtg
gtgaccataccagtggctgctgacatctcctgccatgggaagcataattgcctccagcagcctcccccttagat
ccatcatttttgttgcacttggcctgggctgtactcccggccaatgactgaacatggtgagcatagtaatgcag
gcccatttctgtgaggagcaggactcctccagtaggtgactttggctcaaggactctctattggcctggttgaa
cttttcctgaactgtgctactgtctgagactcttcttacccaatcctctttctcgccccaattgtcacagacca
cctgcattgtggtctgagtctctccccaccttctcttgctcttccctgtttatctttcacaggcatttccccca
gtacattccttgaatgtctaacccgatacgggtgcctgacttttggcagacctaagcagacaaaaaggagtact
tggttacctagctcttctttctaccacaaacatcgagggaaccctttttccctcacccctctgccacaccccca
ctgccccagtgaacaaccacagagagagctgtggtataatattaggctggtgcaaaagtaattgcggtttttgc
cattacttttaatggtaaaaaccgcaattacttttgcacctacctagtatttgtgtccccccaaattcatatgt
tgaaacctaacccacaatatgatgtcattaggaggcaagaccttgaggaggtgattagatgatggggtggagct
ctcctgaatgagattagtgcccttataagaagaagcccaaggaagctaccttgactcttccatcacatgagaat
gcagcaagaaggcaccatctactaatcaggaagagagctctcaccagacactgaatctgccagtgtcttgatct
tgaagttcccagcctccagaactatgcataatgcatttccattgtctctaagccacccagcctatggtattttg
tcatagcagcctgaactgactaagacagtgagecacatgagaagtgccccaacccctcccttaagcacttggct
cacagatcagtgggttcatttctgcctgagttttattgttattctgtagatttcttgggctagatatatttttc
tgttattttccttcttcacctcagtcatgaattggttgttttaaaaaagacaatgtaagtcatggggaaactcc
tgacaactctactctcctagggttcctgataaaaggggattcagttgagtcctctgatggtctctacctgccaa
agtccagcagcccttagcaaacatgctgctcgtttctgtagagaaggtgctggtgtcccaccatacttctctct
ccctcatgaagggcttgcgacccagcaaatgggtggcttatatgggtctgtttcaaaggaagagccagctctgg
gaagaaaaaegatgagcataagcataacctaccactgtgcctgggaaagcagacaacttttttgatgtgtgaat
atctaatgagaatggaatccatcaattaccttaaacttaggcacagtcttcaaattcaatatatgtgggatata
cttttagtcagtttgtagacgttatttgtaataaataatctggcttctctaaagaaattattttaagtgtttgg
tttggtttgatttaatggtaaaattatatttagtggcagagaattatagcaatggtgataaactatagagtgtc
ataagttcatatcttattctcacatttgaagctgcctgcagatgcattcaagatgcagccagaagtcaggagac
tcaggctgttatttggagctcatcattttacagccttgctggactcccactttctcaggggaaaaatgtggtgt
tgacccagattagctctccaggccctgctgagttgggcactctgtaagctggagggtcttctattgtcttcacc
taagtgtcaatcaacaacccaaatgggcatgggggaagagggagctgggccaatgcccagggtgcctggtagag
agataccttgggcactggaaggcaccagcttcccagagagaagggggagggccatgaaaaagttggctgtagat
gccagggacactgggactctccagctgtgtgtttgtgtcttctgaagacttatgtttcattcctttggagcatg
cataatcatacactgtgggatgtgttatatagattgcttgatagttcaccactgtaataaaatactgtgactgg
aatetgetcccagtctgcctttgatagcacttgtgcaacacacatttactgagcatttacagtgatccaggacc
tgtgttgtgaaaacattgatggacaaggcagatggtggagcacgtcagtgaggatttttaacaaaggctggtaa
gtgctataaaggaacattgtaggacactagagaacaaagaacaggagaacctgacttaggctggggtggggcgt
tggttagaggaggctccttggaggacatgaggtttaagctgtgacctgaggatgaatagatgttggccaggtga
ggtaccggtatttgtcagccttaccagtaaaaaagaaaacctattaaaaaaaaaatacacatacaaagcctcat
cagccatggcttaccagagaaagtacagcgggcacacaaaccacaagctctaaagtcactctccaacctctcca
caatatatatacacaagccctaaactgacgtaatgggactaaagtgtaaaaaatcccgccaaacccaacacaca
ccccgaaactgcgtcaccagggaaaagtacagtttcacttccgcaatcccaacaagcgtcacttcctctttctc
acggtacgtcacatcccattaacttacaacgtcattttcccacggccgcgccgccccttttaaccgttaacccc
acagccaatcaccacacggcccacactttttaaaatcacctcatttacatattggcaccattccatctataagg
tatattattgatgatg (SEQ ID NO: 191)
Exemplary HDAd35 donor vector (HDAd35-T4-Ef1a-SB100-Flpe)
(Ad35 5′end: 1→481; pgk: 14103→14614; SB100: 14763→15785; BGH pA:
15811→16128; beta globin pA (Complementary): 16088→16376; Flpe
(Complementary): 16488→17759; EF1a (Complementary): 17780→18895; and Ad35
3′end: 29751→30158):
catcatcaataatataccttatagatggaatggtgccaatatgtaaatgaggtgattttaaaaagtgtgggccg
tgtggtgattggctgtggggttaacggttaaaaggggcggcgcggccgtgggaaaatgacgttttatgggggtg
gagtttttttgcaagttgtcgcgggaaatgttacgcataaaaaggcttcttttctcacggaactacttagtttt
cccacggtatttaacaggaaatgaggtagttttgaccggatgcaagtgaaaattgctgattttcgcgcgaaaac
tgaatgaggaagtgtttttctgaataatgtggtatttatggcagggtggagtatttgttcagggccaggtagac
tttgaccoattacgtggaggtttcgattaccgtgttttttacctgaatttccgcgtaccgtgtcaaagtcttot
gtttttacgtaggtgtcagctgatcgctagggtatttaccggtattcaaggattacatgagcttagaaatgtaa
ttagcatagtgtgtggcatagtgtagataccaaataaatatgatctctccttctactcttgaaaatgcaaacac
attcttggtggtcctaaaatagcctgtaacatggtttactcagcagcatttgctattcaaggcagatctgcctt
tagtcattggctgcgctcctgaacagctgtgtgaaaggctaacttttgtaaaccaaatcaaaataaaatgcagc
aaaaatttgtcactgaaaggaaatcctcagtatatccttttatgaaatgaaagatccctcatccaaacttaact
tttttaaaagtgcgcatttggagatatagccctttcttatgaatcctaattcaattttggccataaacacacgt
tgatgttccccaccccaaagcacatagcaacaagagtaggttctatattgaaaataatgacaatttaaaaacat
gtacttatttcactgtatgtggacagtgtctatgattgcatcatgaagtgtcatataaccatgtacgtgtacat
gagagagagatagagagagaagtggtagggtggtggtggtagaggggatggcgatagtaatcatggtaatggta
gaggtgatggaggtggtaatgacggaggtaagggtggtagtgatgatggtggtggtggtaatggtggtggatgt
ggtggtggcaattgggatggtgggatggtggtagccatggtgatggtggtaatggtgttgatttaaagggtggt
ggtagtgaaggtgagggtagtggtggtggaggtggtggtgctggtagcaatagtgatggtggtgatggtgttga
tgagggtgttgggatcagggtgagttcccacagtatatttcattcttgttgtaccactctgtcaacagcaccac
tgactgggacagaggaagaaggcacactctgaatgtgttattaacagaaacctcaaaacagtctgtctccttgt
agtcattcaaaattatctttttcttacctggaaaactgaaactgaattaccgggaaaaacacaggagatttttg
tttgttaatatgctgccaataaagtaattttatgtcaaatttaactacaggaaagggcaaggcattttctaagt
tccttagatgtcatgtggctaaaaaaaacaaaaggatggacageagttagatactgtacacttagctgtttgaa
gccatatattcagaaagcagatgttgggagttggtgtttgaggactgatttcctggaggtattttatataggcc
aagttcattgttctaaactctaagggcttgacttgagggaggaaaagaggcaagaacatgtttagttttgctga
cagcatcacatgggcagccctaaggctagacaactttagggcctgaagcttattctaggaaagaagcacctaca
gagtggcactgggctcccctccactatagagatgaagtcatatgacagtaaagggcaggcagggctgcctaggg
ggcccagaactgacacttccattagaatgagcacaggccagggagagaagtggggaaccagagagaaggagctg
gaattctagtaggacaaacggtaagtgaacaacaagaacaagttaagagtgtgtgcagtattctttcaaagact
gaaaaaatagtgatgtgatagaatggcaggtggctctgagcaggccaggagaaggactgggggcagagcatccc
aggcaggagggcagcaagtgggaaggccctggggtggggcttttggactgttccagtgacgggcaggcagccag
tgtgcctgtcacacaatgcaccagggaagtagtcgtgaatttgcagagggtcttgcaggctatgggaaagggat
tggattgtattttgtttgtagggaagccatcgggggacttaagcagaggaaggattggcttcatctctttgaaa
aagttctctctggatgetgatgggaggagaaatggaaggaaaagaaacacttttaggggcaagaacttttgaga
agggtggaattgggagtgtggagttggggccagctttggcacaggaggggaagctaaacacgtggccgcatgag
ggcctgtaattctacctgaaatgggtaccatttgttagggtaaacaaatgaaccaaatgcccagtgatacagac
caagtgttggcaaacttcttctgtgatggcccaggtagtaaatgtctcaggcttcgcaggccatgtggtctctg
ttgaagctctgtgtagtagacaatatgttaatgactgggcgtgactgtgtgctaataaaagtttatttacaaaa
acageeegtgggetggatttagetcacaggctgtagtttgccaacctctgacctagagcatgaactgagcatct
tcttggagggaaataagttctttccaagttgccctcctcacattgcagggggccatgtaggcccattattcaca
gaagagtgggtgggcaacctttctggagcagaaaaacgtaaagatttcttccgtagtgcaagtaaggtgaccat
ttctaaaccgtgcaagtgatccagcagtcccaaaagttgtttcacttctcattgtgcgcccgttctcaggtgct
ccgaagcttccagtcctttgtagggacatggatgaaattggaaatcatcattctcagtaaactatcgcaagaac
aaaaaaccaaacaccgcatattctcactcataggtgggaattgaacaatgagatcacatggacacgggaagggg
aacatcacattctggggactgttgtggggtggggggaggggggagggatagcattgggagatatacctaatgct
agatgacaagttagtgggtgcagcgcaccagtgtggcacatgtatacatatgtaactaacctgcacaatgtgca
catgtaccctaaaacttaaagtataataataaaagaaaaaaaaaaagagaggagagaaacatcatcccctccag
gatacccttgggccttgttcttatagtcttgtacattgttgaacaatttgcatgggctagtggattaaagcaca
ccctccaccctcaggccctcaagggtctctatgataatacagtctcaccttctaccctttccatcaccatccta
ggtgctatggccaaccttgaggctgccatgttaggtctatgcatttcccacctccaccacataactctctgaag
gccaggtagtttcctattcatcttggtaaccccaaagcctcgtgacagggctcagctggcatctgcggatgtga
atgaaccattggagaaaatggtactctgcaaataactctgttattttcccatttcctgtgtaaggcctagagac
aatgactttttaattgcaccccttcccctctgtatgacactggccttctcttgtgtccagcaatgtgggtggcc
tagatgatttctaagggacttctggccaagatgaacagcagctgcatcttactgagcatttactatgtgccata
tactcagecacagetctaggggcatagaagcaggagctctcagggtcagggcagtgagtgageaagcgageace
tatgccagccctgcctctggatggggacttgagagggtgatggaagcctgcagcactggagggaggcagacaaa
gacaggcctgtgetgagggggcceggageaagagagagggaggcaatgacagcagagacatgcetgcgccttgg
gtttgagtgcccagtggtcaaatccacttccctgtggctgatgcttgcctttctaactttggaatttaggggtt
ggagatctggtgagaaggtaggagggagatgaggaggagaagggaaaggcaggaaggaaggggagggaaaggaa
aagcaaaaggggaggaggaaggtttccaacaaattattctatatcaactgcggaaatcaaaatttgttgcccaa
atcttagaagctcatgtccctcctccccagaagtctggaatgcagcactccaggggtagcttataacccaaata
tctatctgtaaaaagagaaacattgggctttcgagctgtggattctcagtaaaagcaagaggcctcagcctaca
caggccagcccagagtttgaggaaccccaggcccacacccacagggctggcccctgggtctgcatactccctag
aaatgtgcacacttctgagcctcaactctgtcctggagtctaacagcatccctctccttcctggggcagttcca
cctccagaaacctgttaccttgggccttatgtcaaggaaactgtgggaaagagctaggcaggaatgcagatgag
gccagcatgggctcctaaaagtttagaaataggcagtgtcatgctcccaggtgcctgcataaaccagctgaaaa
atggagctcccctcaccagcactctcccttcaaacagactgtgatttgcaggtcactggtttaccaagccaggc
tacccaggcaggacccagatgccaagcccagtggtgtcctgcaagctgagcagtgctcagttcttgcaaaaaaa
ggtctgtgtgaaggcaaggcctctgcctggcttctcaccccagttgggtgtctggaacaggaaggagcccttac
tgcagaaaaaggaggagggagcaaagggagcgaacagctgcgtgctccatggggaggatccccaaagtagaaag
gcgcatacacactgcagcccttgacccagaatgctcacagctacattacagattcaggtctcctcagtgtagtg
gggctgctgatgagactgtggcatcctcaggggtcaggacacacattttccatcactcttctgatggcaaaaaa
cctctgagccaatgccaacctctgatcattaaaaaaaagtgctcacagcagtgtgtggtttaggatcatgccct
gtgtggtttggaacacgtgcacaaccacaccttgttcatcaccatcccagaaaccctgacgcaggcaaagagca
gagttattaaccctactttactgatgtggatactgaggcccagaggctcatgcaagttatcaataagtggcagg
gacagttgcctctagattaactagcccctaggatcacctgggtcttggaaggggacccataaacatgagctccc
ctctettggggccagatttgcacctgtgccgcgccttcagectgcatgaagtaggggctgetggcaaagactea
aagctgtaaatctgggttttctcttgaggcttctaagggagctgtttcgacaactcactctgttcccagctggc
tgcccctgcatagggttttaaagcagcctagctttctgccaggcttggcagtggacaacgctggtcagaacatc
ccagagagctaccagaatgaagtaagtttgcttctactctttacctgtttatgggctgtctctgccactggaat
gaaaggcactgagaacagtgcctggcctgcagaaggccctggaaatacctgagctcctaatctgggaataggag
taggaagagctttggaggcagggcacctgagtttgagatctacaacttcctgcctgtgtgacattgggaaagtc
tccatcctttctgagcctcagtctccaccctggggaagtggaaatatcaatctctgtgacacagaagcaaatga
gcgaatgtgcacaaagtaccttgcacaagagagacgctcaaacacttgcctccaggtttcaccgagaactacag
agtaagatagatttgttcccagtggaggaagcctgggaataatttgcccctagactatgaattcctggggctca
agatcgagcacagggccaggcacacagaagggaccctggaaatgtggcaggaggccagagatagacaggccctt
agagctcatacccatgccctctgacctcaagaagaaagaaacctgctcaaaatctcacaaagagcttgttccaa
ccctgaatcgagtctgaggactccttcctgagtccagcactttttctgcaagaagtatatgcctccaaagctga
tgggcgcaaatcttgaaccccgtcacataaacacaaagggaggaggtgactagagctcctcctactggatatgt
ctaaggtcaccagtctaaagaaaagggatggatagaatgaggccagtatttttgcagccatccaaatgtccaca
tacgctgttacactgagggctcctctctcccccgtcttcagccctacttgcatttagaggtgagaaagatatgg
gctgaggggttgtttttcatcgtattgtagatggaaagcacactgcccttggggccatccaaatgtggaccttg
atgtagcaccccaccttctggatggccatccttctgaaagtcactgaatttctcagactttattctctttatcc
ataaagaaggagaataataataatccccccaccctgcccaaccactgactggttgggaagctcagaagaaatac
tgggcacggcatcccattgtaatctatagagtgagtcgcttcttaatattaaatggctgaacacagaagatgtg
caaaaagtactgtgtccccttcctcctccaactgaacatttcatgccctttgcaccctcattttgtctaggagc
tgccttatgaagggaataggtacctgctccgagctggaggaatctttgccacttatggtggggtatggactgag
acagagatggcatgtgacatgcgcactgagtctcaactccatgcaggctctggagcactctcaaattggagtac
taatgccttttaaattctcacactagcaatcctttgacctactgatctagggatctagggaaagaatcgtgate
ttaacttcaaagggaaggacaaaatgttctgcctcctgttaaaactccatacactaagtgcagagactggatgc
cttattaaccttgggtagatgcccaaatgttcaaaaggtcaaactcttctgttccccagatcgccagagtcatt
aaccagtcacactattaaatgaatgaacagatgctgaaaaggtacttgcattactgagatttcttatggtgatg
gcccctgcctgatatgtattcagcattttgtagttttcaatgtgcattagagtatagtggtgatgacattggcc
tctgagtttgccacttcttatatctgtgactttggtcaaattgcttaatctctctgagtctcggtttcctggag
ataataatagcttcttcttcccagggttatcatgaggattacaggagataatgccccaaaaatgcttagtaaag
tgcctagcacctagtcaatgctgaattaaaggtggttattcttacttttcgttcatttgaactttgttctcagg
gagggcaaaggatagacaaagccccatagctagtgaggagtagctgcaagactagaacccaggtgttctgagcc
ctagtcttaggccaagaacaactgttacgtgagatgcacgttttccttcaagggagctcacaattatttccatg
taaattcaaggactgctaaaagagaactctcctctgggactgatatcattttatttcaagattgatttgaaaca
tgttttttgtttgtttgtttgttttctaggaaagaacaagagaaccagttaagctgaatgcctgaagcaaatcc
ctgttagcgatgttttcaggatgagggagagtggtgcaagaaacgtgcttccagatgcacatggtttcctggga
ctagggttcagggtgtcatccctgggtgttattaagtgtcagaaggagagcaaacaagggaaacatctgagatc
cagctaaggctacaccctggaaatgcaagcccagctcttgcaaaggacctcctttggccactcaccttccaggc
cttacaataacttgtttggactgcaggtttcttggtggactcacaggccattctgcttttatttggtcaacctc
agttcacaagcacccagatgctgagatcctcagcatgtgcagcagagtttcatattagcactgggtacctttct
gaggctacagggataccgtacagcagcacctgtcacgtccagccaaaggagtgggctctctcaatgtcatccaa
tgctgtttcaactgtgaagaagaccatctgagagagttgcttttggaggctgaggcaaatttttaaaattcttt
gttctcctcaactggggtgaattcttggtcttctaggacagcttgaagttttagaaagagtcaagccactcaga
accaacagagaactctttcagagaacaaggtgtggcatagaggaggcagagggctgatcttgatcaaatccaaa
gtgtgactctaaagcaatgaatgtgaatttttggcaaagcttacaaagggctctaaaggccatctgcaaagaga
agccaagcctgatcgatgaatcactagtgcggccggatatcgatcggcacgctgttgattttctcatagtaagg
aacagtgggccctttcagtcccacttctgtagtctgtggtactacaaatggtgagcccatgatgttgccattca
tagggttattctccagcagtaatgactggccagccactcccatagccgcggggctaggatttattgtcaatgga
gggacctgcagttctgcacaagcagtactaggatgagcacctgggcccattgcaagggtgacatcttcaaggca
aggcctcttaattttattagggtagcccccatcagecatgtctggaaactggaagtggtcttcttcttgtctcc
tcttaacagttccctgtgaatggaagagaagagaggaggagaagagaggagaggagaagggaagagaggtgaca
cacacacacacacacacacacacacacacagagagagagagagagagacagagagaaagagagagagagagaga
gaggaatttttataaaggtttggcacattaaagctaatgaacaggaaatgtgcatgataaaacagacctctcag
tttaaagacttatagttgtgaaaactataaaatacagcctgtctttggaaccatagtgcttatttattcattat
tatgtttcatctaaactgtctaattacatttcaaataaggcattatgttgtctgtatactaaaacgggatagaa
cgttattcaaagggtaatctgcccacttcaaggagagttcaacaaaactatgcagaagtcactaaatgaaccat
gctgccaaaggcaggcattggagagaaaactagaagtagctaaatagttttaattctttcctgtctacagacac
atagattttaacgaaggaataccatagtatagaattgaacttttaggctgccttctagtcttggttaaatgcat
caggctgcagtggtaaaattgaatacaacagagcccttacaggaaagaagtagatctggatgtgttttcttggg
gagctgtttaaaatactgtttttgggaaagcacaagtttcagaacagtcattgtaggcatcgtattcattgttc
catttatttttacacacacacacacacacacacacacacacactctcacacattgctatgtgtacacaaaaata
atttggaagaacctatacccaacaatttggagtggtcatttatttgggatgactggcaattccctttctattct
cttcatttctgcttgtttgtctttaacgagaacgactcataatccaaaaatttaaaaaagtataaagttatcta
aataagaaattttcctctgaagatgcatcctcaggttggggagatattaaacaatgagaaaaggccccaatctg
ggatctgaaccttgggggagctgcccatcatttatagaagcacagcctttgggaacaaagcaaagtcactagca
atgtgagacttcctactcttcatggcttcatacagtcatccatcgctgttgtgttaatgaccatgacctgtatg
ttagcaggtaaatgggaaaggaagtgggggcaaaggagtatgtgcaggaatgatcaaaataaggaaaggaagag
agggatctggaaatcacctgaatgccgataggtgaacaggtagaattcttttaaagcttcccccacccggtacc
ccccaaataacccctttccagctttggaagtttcactaggacatacagtgetcatoctetgatgtcacettaag
tttggctcttctggtttgatgagcttgtagcccactaggagctcaaggcatgcatggggccacttgccagcacg
atgaggggcatgactgtcatggccaagtgaacatcaaagcagatccccagggctgtatgtctcaggccttggtg
cacatcagaatcacttagaaacatccacattcctgggccctcccaccacaaactgacagcttcatccagggtgt
ggcccaggcatcgggagtttttccaacagctccatggctgattctcaacagaaaaccactggcccagagcaagg
gtggaggcagcgtggcatagggctctgaccttggccttgccactgaacctctcagagccccagtttctttatgt
gtaaaatgagtgtaattatagttcttttctcatgaaggtgctctgactattaagtgaaacggggcacattgtat
gacacctaatagctcctcactaactggtacccggcattataaagggcaggtatggaagggttctgggagtccaa
tacccttcttaaagacagagaggtctctgagacccagagaggggcaggccttacccagagttgctcagccagag
ggcaacaaggcccaggtcagatgcagggcccctccaccaccactcagctgcctccagacccactgccttcgcca
tgttgttggtaggacactgcatcgcccccacagaaggggcttgccaacttgagtgagaggacttgcacacttct
ttgacttttcttttgagatgcccacaatctgaacaagggcacttcaagggacagctctgtcaccaaactcatct
gaggcctgaataccatgggtcaggcaggaatgggttggagaggtgtagagcaggcacaataagagggctgaggc
ccatgcagtcatcagtgcccactttcccaggagtctgactgggcacagcacccatagtgtccctgagctggtcc
atggagcagctcactaactgtttggcccacagcaggtgctcagtaaatggcagttgaacgaatcaatggacaaa
ggaacataaattacccaacacacagggagctcagccatttactcaatccattatggagtaacctacaaacaagc
cactgggtcccaaactgaaattgtgtctcttctacattctcccaaagaatccaataggttaaaaatagaaatgt
atgaaatagatcaatcagggatgattgcatgtggatttgacataaggatcccctgcagggagtctgagctggca
acagtcaggcccaaagtgctgtccatgatgtctcgaactgcaagacagttttaacaatggcgaagcaatgcaga
accaggcaggccaaggagggggtgggggttggggaaaggaagggagggaaggggctgtgaggggcaatggtctg
gcatccctgccacgtgagcctctgaaatttgctggcagcttctatgggctcccagagctttcacttaattgttg
gtctgccactaacctgctgggagtaaggtgcagggatggaggaggcagggcatgaccaccagacactaaaggta
ccagctggggccactggcaaagggaaggaggctgcacctctcctacatgagagcccgtatacacacaccttttc
cagcactcatcaactgcatcccaagcaaatggtccctgatcaattccaattctagaaaccaactgactactcaa
taacaaagtagatcccagcaggccgccactgctggagcggatgccacttttgctatgccaagtctgtggctgga
cagctgctggcatgtacactcactgactttcataaggatgcctaataaagggggcaggctcacctggcttttct
caggggtggggtttggggtgccgatagaggctgctgttttggcagagtggcaagctgcaagcctcttctgagct
ttcatttttcaatggacttcagtgagaattcactttgtcagaggccatgcagctccatgttttggatttcatgg
aatgagetttcaacagtgagectgaagtgccctggctgaacageaagaacaccagecaaccctaaacaaggceg
aggagaggcggctgtgtttacacggaaggctcagccttgctgtaatagcgtctgccttcaccagacatcagtga
ggcgtggaaatctattatccagttaattttgcccctagataaagacttgctttcgtgtcttctctttcacagtc
ccatgatctgttactcatctcaactgcgagaagttggctgggctttcccctgtgcccagtgccacactcgtgcc
ttcactgggtcacctgtgcctgtggctgatgccgctgaggttttgcctgcccagactgggtgtttctgactaaa
tcccacagccaccattttagatcaagggcaggagatagctcactgctccggaatgacctcccctcccagaatcc
tggtaggggcggaaggtccccaaccaagctcccagccctttctaaatgaatctccctgcttcacccatgtgctt
ttctccagtctctgcggtcttgatgacagcagggtattagtcctagctgtcccacagctcctacttctttcagg
cctctccctgtgacaatcagtagccactggcaggatttcctcagagcatatctcgatttgctttcagacaatta
gttaaaaggacactggaccccagacgtcccaactcccagccagagccctcacaggcccggcctttggtggtgag
gaagggggagggagtgagtgacagtgccctggcatcttttagaaacgaattcctttctctccatacataaatgc
ctgcagagtcccatttcagaatccggcagacaaagccaccaatgtgatccccatgaccttataaacattcatta
aaatgcatttcaaggcatgtgatggcctccccaccccctagataatgagaaaacaaaggtttctcttctgatag
agacaagttcagctctgaagtcaacattatttctggttctgtctgaacaatgacatatggcaactcttcccttt
ctatagttctagtccagaatgacaaaaaaggggaaaaatttcttagagaaggtagagattatacgaatacagtc
catgaaatgagcataaggagaataaagaatataacttatccaaagaagtctggcaggctgttataaatgcttga
ttttggacactgtagttggaggtttaacatggacaccaataaaaaggtcagcaaagggtatgcactgttcctat
tgggcaagaagataggaggtcaaaggtaaccaggaaagataaactcagggagacttattttccctccagagggc
actgggcttgtaggccctgggcaaaattgtcaaaaaggtgaaaatcgcctgtggtttatttagtctgctctttc
ttcactagtgcctcaccagttcagttcaggccaatttgetaggaattctaccgggtaggggaggcgctttceca
aggcagtctggagcatgcgctttagcagccccgctgggcacttggcgctacacaagtggcctctggcctcgcac
acattccacatccaccggtaggcgccaaccggctccgttctttggtggccccttcgcgccaccttctactcctc
ccctagtcaggaagttcccccccgccccgcagctcgcgtcgtgcaggacgtgacaaatggaagtagcacgtctc
actagtctcgtgcagatggacagcaccgctgagcaatggaagcgggtaggcctttggggcagcggccaatagca
gctttgctccttcgctttctgggctcagaggctgggaaggggtgggtccgggggcgggctcaggggcgggctca
ggggcggggcgggcgcccgaaggtcctccggaggcccggcattctgcacgcttcaaaagcgcacgtctgccgcg
ctgttctcctcttcctcatctccgggcctttcgaccgttgatccggtggtggtgcaaatcaaagaactgctcct
cagtggatgttgcctttacttctaggcctgtacggaagtgttacttctgctctaaaagctgcggaattgtaccc
gcggaattaatacgactcactatagggactagtaccatgggaaaatcaaaagaaatcagccaagacctcagaaa
aagaattgtagacctccacaagtctggttcatccttgggagcaatttccaaacgcctggcggtaccacgttcat
ctgtacaaacaatagtacgcaagtataaacaccatgggaccacgcagccgtcataccgctcaggaaggagacgc
gttctgtctcctagagatgaacgtactttggtgcgaaaagtgcaaatcaatcccagaacaacagcaaaggacct
tgtgaagatgctggaggaaacaggtacaaaagtatctatatccacagtaaaacgagtcctatatcgacataacc
tgaaaggccactcagcaaggaagaagccactgctccaaaaccgacataagaaagccagactacggtttgcaact
gcacatggggacaaagatcgtactttttggagaaatgtcctctggtctgatgaaacaaaaatagaactgtttgg
ccataatgaccatcgttatgtttggaggaagaagggggaggcttgcaagccgaagaacaccatcccaaccgtga
agcacgggggtggcagcatcatgttgtgggggtgctttgctgcaggagggactggtgcacttcacaaaatagat
ggcatcatggacgcggtgcagtatgtggatatattgaagcaacatctcaagacatcagtcaggaagttaaagct
tggtcgcaaatgggtcttccaacacgacaatgaccccaagcatacttccaaagttgtggcaaaatggcttaagg
acaacaaagtcaaggtattggagtggccatcacaaagccctgacctcaatcctatagaaaatttgtgggcagaa
ctgaaaaagcgtgtgcgagcaaggaggcctacaaacctgactcagttacaccagctctgtcaggaggaatgggc
caaaattcacccaaattattgtgggaagcttgtggaaggctacccgaaacgtttgacccaagttaaacaattta
aaggcaatgetaccaaatactaggggccctaaccgcggggatcaacgcctagagctcgctgatcagectcgact
gtgccttctagttgccagccatctgttgtttgcccctcccccgtgccttccttgaccctggaaggtgccactcc
cactgtcctttcctaataaaatgaggaaattgcatcgcattgtctgagtaggtgtcattctattctggggggtg
gggtggggcaggacagcaagggggaggattgggaagacaatagcaggcatgctggggatgcggtgggctctatg
gcttctgaggcggaaagaaccagctgggggttacctactttctttatgttttaaatgcactgacctcccacatt
ccctttttagtaaaatattcagaaataatttaaatacatcattgcaatgaaaataaatgttttttattaggcag
aatccagatgctcaaggcccttcataatatcccccagtttagtagttggacttagggaacaaaggaacctttaa
tagaaattggacagcaagaaagcgagattagatccctattatttttgacaccagacaagttggtaatggtagcg
accggcgctcagttggaattcgctagccgcacatacagctcactgttcacgtcgcacctatatctgcgtgttgc
ctgtatatatatatacatgagaagaacggcatagtgcgtgtttatgcttaaatgcgtacttatatgcgtctatt
tatgtaggatgaaaggtagtctagtacctcctgtgatattatcccattccatgcggggtatcgtatgcttcctt
cagcactaccctttagctgttctatatgctgccactcctcaattggattagtctcatccttcaatgctatcatt
tcctttgatattggatcatatgcatagtaccgagaaactagtgcgaagtagtgatcaggtattgctgttatctg
atgagtatacgttgtcctggccacggcagaagcacgcttatcgctccaatttcccacaacattagtcaactccg
ttaggcccttcattgacagaaatgaggtcatcaaatgtcttccaatgtgagattttgggccattctttatagca
aagattggataaggcgcatttttcttcaaagccttgttgtacgatctgactaagttatcttttaataattggta
ttcctgtttgttgcttgaagaattgccggtcctatttactcgttttaggactggttcagaattcctcaaaaatt
catccaaatatacaagtggatcgatcctaccccttgcgctaaagaagtatatgtgcctactaacgcttgtcttt
gtctctgtcactaaacactggattattactcccagatacttattttggactaatttaaatgatttcggatcaac
gttcttaatatcgctgaatcttccacaattgatgaaagtagctaggaagaggaattggtataaagtttttgttt
ttgtaaatctcgaggtatactcaaacgaatttagtattttctcagtgatctcccagatgctttcaccctcactt
agaagtgctttaagcatttttttactgtggctatttcccttatctgcttcttccgatgattcgaactgtaattg
caaactacttacaatatcagtgatatcagattgatgtttttgtccattgtaaggaataattgtaaattcccaag
caggaattaatttctttaatgaggcttccagaattgttgctttttgcgtcttgtatttaaactggagtgatttg
ttgacaatatcgaaactcagcgaattgcttatgatagtattatagctcatgaatgtggctctcttgattgctgt
tccgttatgagtaatcatccaacataaataggttagttcagcagcacatgatgctattttttcccctgaaggtc
tttcaaacctttccacaaactgacgaaccaggaccttaggtggtgttttacataatatatcaaattgtggcatg
gtggaagcttggcatgggaattcctcacgacacctgaaatggaagaaaaaaactttgaaccactgtctgaggct
tgagaatgaaccaagatccaaactcaaaaagggcaaattccaaggagaattacatcaagtgccaagctggccta
acttcagtctccacccactcagtgtggggaaactccatcgcataaaacccctccccccaacctaaagacgacgt
actccaaaagctcgagaactaatcgaggtgcctggacggcgcccggtactccgtggagtcacatgaagcgacgg
ctgaggacggaaaggcccttttcctttgtgtgggtgactcacccgcccgctctcccgagcgccgcgtcctccat
tttgagctccctgcagcagggccgggaagcggccatctttccgctcacgcaactggtgccgaccgggccagcct
tgccgcccagggcggggcgatacacggcggcgcgaggccaggcaccagagcaggccggccagettgagactacc
cccgtccgattctcggtggccgcgctcgcaggccccgcctcgccgaacatgtgcgctgggacgcacgggccccg
tcgccgcccgcggccccaaaaaccgaaataccagtgtgcagatcttggcccgcatttacaagactatcttgcca
gaaaaaaagcgtcgcagcaggtcatcaaaaattttaaatggctagagacttatcgaaagcagcgagacaggcgc
gaaggtgccaccagattcgcacgcggcggccccagcgcccaggccaggcctcaactcaagcacgaggcgaaggg
gctccttaagcgcaaggcctcgaactctcccacccacttccaacccgaagctcgggatcaagaatcacgtactg
cagccaggggcgtggaagtaattcaaggcacgcaagggccataacccgtaaagaggccaggcccgcgggaacca
cacacggcacttacctgtgttctggcggcaaacccgttgcgaaaaagaacgttcacggcgactactgcacttat
atacggttctcccccaccctcgggaaaaaggcggagccagtacacgacatcactttcccagtttaccccgcgcc
accttctctaggcaccggttcaattctagtatcgataaataggggattacttgaacatagactgtgggatccgg
tgtggagtgcgggagactagcaaagtgaatcctgagagtagcaggtctgcacctgttggatcgagaaaggcggc
ctacaattctggtcaaatgagctgtgcttattgacatattctattagagagtactaccaggtcaccagtcacca
gaaaggctgccagctctccaaccacctccagggaactatcctgaatggggccttaacaagtctaagagagggtt
ggtttgggtcccaagccaatatttgctctgctttatgtcagtcatatggaacccaaaccaaccctctcctatgt
gcctcaccagtcggtgcagggatcccaatttcaagtttggttttttatggtcaaagtccagcatagattaaatg
aaggggtgtgatgatggtgttaaaagagaactccagaccagtttaactcttggacacacatcccatctcaccat
ggtgcttccaaccttccagagatgatgggctcctattttctgatgacaaagccctccacaggattgctgcctgg
ccatcagggagtgcctctgtaactgaggctgagatcccactttcagtcctccagctgtggcccatccctgctcc
gcccaccgggtatggcctgtcctaggctcttaggtatggctgcattgtgaaatgatggctacagagctggcatc
teetgtagtetggttcatctagtgcactacctcatagttaaaagaaatctgtttaagccactgagggtggctcc
tagtgccaactccaagaacaggaagcttcccttttttgggaggaggggcagatggtaacatggatcgtccaggt
caatgggagcagggcaaccacagtaagtactggacaacaacacaaaactccatgtgtggcttccatcgagtccc
tctccaattggtttggtcttctccgtcccatgcagcactttagcaaggggcctggctgaaggctatgaattgtg
tggagcctcctcattgcagtctccaaccatctgatgctgggaaaatgtcaccaggatgcagccatgccgtgtgg
ccaatgaaccgagaaaacaccccttttctagaatgctctaaagaggcagaataatccagaggtgaggaaggaaa
tactccaccagagacccaggcagttcctacaaaagccagactttccttcacctagggagtgacaagaccagtgg
aaaacactctcaagcagtaacccccaaatgctctgcaagccagtggcgtccagataccgcacaagcgagtgggc
tgtctaatcccatcatcatgatgtaaatatctctaggctgccctgggctgtgcctgaccctgtcttcagctttc
cacacctccacctacagcccatgcacagaaggaccacccaggaatgctgcaagtgtggcacctccagggccacc
cagggagaaggagggcagctatgctggtggctccaggcccatttggcgggtggtaccttcacaccacaaagccc
aaactgaggccccagatttggctgatgagggcatattggacaggggtcacttatgctcttccccattgccacct
ggcctctggctacctggacttggctacctgtggatcctctcacaggtgccaccatcttggctgagtctccagat
gcgaggtccctgaggcagtggcgggcttctcgctaatgctgatgggattaggaatgggataggtggggagggcc
ctggactgggccctgatgagecaagtgggtttttagaggggctactggtacatttcagggacaggacatctggt
agagctaagctggggcaataaggagccactgctaatctgagagctagaaacaatcagcttctgggtcattatta
attagggtagtttgggctgtgtggaagtcacgtactatatggggtagccacagctctctctacagataatctct
aagacttctgattgggactgtgtgaatgcagtagcaatatctcttcttactgccaggccctgccagtcctgcct
ccacgccctggctggccccccttatgatctgacccatgccaggctgccatagtatgttacttctgcattagcac
tccttgggacctgcctctccactgtccctcagactttaaagaactatacaaacccaaggggctcttcccaagag
aattgatatgacttgaggtgattccatttctggaagtagtcactccattttctgcctcactctttcagtgcttc
acagagcaggttcgaacgaaggagccatccaactaaccgtcatgttcgggcaaccgaagaagggagtggcagga
tttcctttggagacttctggaattagacagcagtttaatgcaagcatctaaattctctccctcccagagtctca
ttaaaactacagtaagagtttgtgttttgttttgtttttaaagacaaaatcccaccaggatagagagaatagga
gaggagataacagcatcataatttatgaaactaaaatgcagatagaccaggattaactgactacacagcaccaa
ggaagctgaatcacaagacagcagaggagaaaactggaaaggatcgtggtctatacggcagaatcttcccaagc
ctcaggaggaggagctctagatgttcccagatctgggaggtaaagtggaatggggggacatggtcagcgtaatg
gggttgggctggaagcaggttaaggagcaggcagatctctgaatcccctctctgactctgtgtccccaggcatc
tgcctgtcccccaccctggaagaggtctggcttgaccctttgtctggtgaatttcctgctctgctttcctggtc
ctgctggccggatcagtggaggccactcacttcaccccacagggatgttctgtgttgccctacacctgggaact
ggaggtactggaggcaggctgtggtgagcttgaaagcaaaacacagagggcagtccaatctctttggccatatt
tcttctgcatatccaataccatgtccacaactctgctagtgtcctgatggtggtgggctctacacattcccggg
aagctgaaggcagataatgaccaggacaggtcaacctctcttcttctgaaagccttcatctactaatggcctgg
gactcttcccttaaatgcttagattgtgtcttccactaaggttttttgctgttgctgttgtttgtttgtttgtt
tgtttgtttgtttgtttgttttgagacggaatctcactctgtcgcccaggctggagtgtagtggcacaatctca
gctcaccacaaccttcacctcctaggttgaaggggttctcctgcctcagcctcctgtgtagctaggattacagg
cacatgccaccatgcctggctaatttttgtatttttggtagagacaggatttcgccatgttggccaggctggtc
ttgaactcctgacctcaggtgatctgcctaccttggtctcccaaagtgetgggattacaggtgtgagecaccac
acccggccaaggtttttgtttgtttgtttgtttgtttgtttgttttgtattgaggcagggtatcactctggtca
cccaggctggagtgcagtagtgcaatcacggctcactgaaacctccacctccctggcgggctcaggtgatcctg
ccacctcagcttcccaggtagctgggactacaggcttgtaccaccactcccagctaatttttgcgtttttagta
gagacagggtttccccatgttgcccaggttggtctcaaactctgggctcaagcgatctgcctgcctcagcctcc
caaagtgctgggattacaggtgtaagccaccgtacccggccccgccactaaggttttgaaaatgaagcaattac
aagtttaagtctattaataagtgatgaagccatgtagaaaagcagaataattatcttggatcaggaaggtcaca
tgaggatctacttgggggttgtcaatattctatttcttgacctgatcagtgttgacagcaggttttaatttttt
acttctttttgtttgtttgtttttgagacggagtcttgetctgtctcccaggctggagtgcagtggtatgatct
cggctcactgcaacctccgcctcctgggttcaagctgttctcctgcctcagcctccccagtagctgggattaca
ggcaggcaccaccacgaccagctaatttttgtatttttagtagagactgggtttcaccatcttggccaggctgg
tctcgaacttctgatctcgtgatccgccctccttggcctcccaaagtgctgggattacaggcttgagccagcgt
gcccggcccattttttacttccttattaaactgtacatataggccttgcacacttttctgcatcaatgttatat
tccacaataaagggaaaaggtatatacacaacttgataccagtaatgtgaaacatatatttctacatagaaaaa
aaaatgactgaaatactgcactccaatgtgttcacacagtagttgtttctggattatttatatattaaatgttt
atatattgtattatgccatgaggtttgtgttttctctccacttttctgcattttccaagtttactacaaagagc
acatattactcttataatcagaaagtcataaaatatatttaaaaagacaaaattgaaactaataaggatcaaca
caaaacagatgagecatctgtggaaatccgcacagaatactacctaaagagattggtgacgtgcatgatctcac
taggatgagcacaaagcttgccagagcctagggtctatttctagggttggctcttggaagccaggatagttgtt
atctctgggaagagggaggggcacacaaggggcttctaaaacattctgaatgttctatttctgaacctggttgg
tgggtacatgactgttggttttattattatatgttttatatactcttccgtatgtatggtgtggattccaaaaa
aagatttcctttagagaaaaccagaatcacataagtagaaaatatggtgctatgttgaaggaacaactcaagtt
tatataaaatcatcatcatttataggcttaaaaagttgctttggaattttggtctaactgacttgtcttttctg
cagcaaaccaegetccttctggacgtgetccaggcagaggggattagggtgggttcaaggctgcaagtacctag
ctcagcacactctcttcaggggacttagagtttgtctggtgttggctctctgagctcttgtcaggaatgccgac
ccttccgaggttcaggatttgaagcctgccttcccaccccagatttggtccacacagacactcaagtatgtatt
tcaactacaaatgacctgtactttcctattactcctctctttcatggtaacctttctggtatccttccttccct
acatttatgggagggggacatcattctctgctctcctgtcactgaaggctccaccttctgtcttcttctgaccc
atctggttttcctggggccacctcctctccttaccaccctaacgcttttgtaacttgaggagaaatgagagatc
acctagtcaggtcatcattctctgtagatgaagaggcccaatggtttgctcaagaattgccaagcgagttaaag
acagagagtatgagagtcagcaagacctacagaaagcatctatctgcactgttttgcagggacttagcctttgt
gtgtggactcctggaatgccacccactaagaaacattgtctgacaccaactccccacttggtaggtggggacac
tgaaactcatggcaggaaagggccttgccccaagccagggcagagtgtcactcatcactctcaattttcagtcc
agggcaccttgttgtgactatcccaaaggcagccactttccctggtctgaaagacctgaagagagaagagaaga
gaaggatggaaggcagagtatgcggctttgattcatttcctggtgaaaacagatctatacgagaagcaaatttc
acgaaagggaagagaagaaagtgtcccatacgttgctggcctgtttcaaccttgctttgattcttgctgaaaag
ggtaccgtgtatttctgagttcaacatgcagaccagtgttaggaaagccactgcacctccactttagcctccag
ggctgtgccctgcaaatggcctgcagccttggtgcctcgctctccagactgcattttggaagatgggacagagg
cttatggaagcccacattagaacgggggagcagaatgggtgagatgagggatccttgatagtgaaccagatgaa
ggaatggtagccaaatgccaggcctcctttgtggcttcaatccaaaggctctggagcccttccagggcagaaca
tcaggcatgtttacccccactgtcctcaacagtgacagaggtgcaatcttgggcagctggccattttgaaagca
acctccttaatctcaactgggaaggctccctagcaggacccctgtgttgcacacctggaggaagctagactaac
cagaagctcagcacggttccatctgggatgcccaggtctgagacgaaaaaggtaactctcttttctgggtcctg
gcccagttgtgtctctctccacctcattctctgagatgcctgtctccccttttttgtcccatcaggaggcaaga
gctatcactgggccagactccaccagaagccaagccagcttgttacccagcttctcagggagcaaagaacagcc
ttgtttctatcttatccccactgtcccctgcccctgccccacctcccagccattcagcttctggcttccccaga
gctgcctgcttctttgtggtcctccattccttgaaaagaccttctagtcattagtgtatataaatggccactta
gcccagattacagtgaggtcaacagctggggctctgagaattgtcacacactggcacaggagaggaggctattc
ttccagagaatttggagggcactcccatccacttacaacaaaaagcccatccactgtgcttggcagtaggtgat
ctgagaaccaatggaaccaggttaatcctgtggcactgttgagtgaggagagcagtggcgggcactggaaaata
tcagagacaaggcaggagacctgaaatctaggcttagctcctcatatacttggcagctgtatgacctcagacaa
ccagtgttacctctctaagcctcagtttcctcatgcaaaaggagggggaataacaacagagcccactgcttggg
ggtgttgtgaggacaggatgaaaaaacaaacagaaatccctcagtacaggattcagtgcagtggacagtcttgc
aaggtctggttcagccctccacccctaccctcaccagtataaagaactctggcctacaagtcagatgacctgag
ttttaatctcagctttgccattagccgtgtgaacttgagaaagtccctttcctttttacatctattgggatgat
catgcattttttgtcctttattctgttaatatagtgtgttacattgattgcttttcatagactgaaccagcctt
gtattccagggataaatctcacttggtcatggtgtataatcctttatacaaatgttgctgggttgagtttgcta
gtattttgttgaagatttttatgtcttgattcataaggaatattggtgtaccttccccttttatggccacagtt
tccctacaatgatgtagtcgaactagacaacctccaatatctttcagtattcatgtcctctgattctgtgaaac
taagaaaattaagaaatagtgattcataggcacaaggcaggcaaaacttagactccttgtagaataattaggaa
gccaaatattcagtgtgcttatttctcaaataaccttagtttctccagtctgccccaactccgaggcctgaata
tctctagatgcttatgatggcaactaaagcctaaaagctaattcattttaaagttcttccaaatgcatagggtt
ttatttttccagacctgggttcagatggggaatttgacaaacaatggaaagggggaaaaacaacaatctaaaca
ctgagtgacaaagtaacaaagaaatagtctagctatcagccagtcaagccagccttggctttgctatccaaagt
agtcagtctaattctaccaccagtttctgttcctgtagctgtctactgcctgccagggactctgccttcccacc
cacaactaccaatggaaggatgtggtgaccataccagtggctgctgacatctcctgccatgggaagcataattg
cctccagcagcctcccccttagatecatcatttttgttgcacttggcctgggctgtactcccggccaatgactg
aacatggtgagcatagtaatgcaggcccatttctgtgaggagcaggactcctccagtaggtgactttggctcaa
ggactctctattggcctggttgaacttttcctgaactgtgctactgtctgagactcttcttacccaatcctctt
tctcgccccaattgtcacagaccacctgcattgtggtctgagtctctccccaccttctcttgctcttccctgtt
tatctttcacaggcatttcccccagtacattccttgaatgtctaacccgatacgggtgcctgacttttggcaga
cctaagcagacaaaaaggagtacttggttacctagctcttctttctaccacaaacatcgagggaaccctttttc
cctcacccctctgccacacccccactgccccagtgaacaaccacagagagagctgtggtataatattaggctgg
tgcaaaagtaattgcggtttttgccattacttttaatggtaaaaaccgcaattacttttgcacctacctagtat
ttgtgtccccccaaattcatatgttgaaacctaacccacaatatgatgtcattaggaggcaagaccttgaggag
gtgattagatgatggggtggagctctcctgaatgagattagtgcccttataagaagaagcccaaggaagctacc
ttgactcttccatcacatgagaatgcagcaagaaggcaccatctactaatcaggaagagagctctcaccagaca
ctgaatctgccagtgtcttgatcttgaagttcccagcctccagaactatgcataatgcatttccattgtctcta
agccacccagcctatggtattttgtcatagcagcctgaactgactaagacagtgagccacatgagaagtgcccc
aacccctcccttaagcacttggctcacagatcagtgggttcatttctgcctgagttttattgttattctgtaga
tttcttgggctagatatatttttctgttattttccttcttcacctcagtcatgaattggttgttttaaaaaaga
caatgtaagtcatggggaaactcctgacaactctactctcctagggttcctgataaaaggggattcagttgagt
cctctgatggtctctacctgccaaagtccagcagcccttagcaaacatgetgetcgtttctgtagagaaggtgc
tggtgtcccaccatacttctctctccctcatgaagggcttgcgacccagcaaatgggtggcttatatgggtctg
tttcaaaggaagagccagctctgggaagaaaaacgatgagcataagcataacctaccactgtgcctgggaaagc
agacaacttttttgatgtgtgaatatctaatgagaatggaatccatcaattaccttaaacttaggcacagtctt
caaattcaatatatgtgggatatacttttagtcagtttgtagacgttatttgtaataaataatctggcttctct
aaagaaattattttaagtgtttggtttggtttgatttaatggtaaaattatatttagtggcagagaattatagc
aatggtgataaactatagagtgtcataagttcatatcttattctcacatttgaagctgcctgcagatgcattca
agatgcagccagaagtcaggagactcaggctgttatttggagctcatcattttacagccttgctggactcccac
tttctcaggggaaaaatgtggtgttgacccagattagctctccaggccctgctgagttgggcactctgtaagct
ggagggtcttctattgtcttcacctaagtgtcaatcaacaacccaaatgggcatgggggaagagggagctgggc
caatgcccagggtgcctggtagagagataccttgggcactggaaggcaccagcttcccagagagaagggggagg
gccatgaaaaagttggctgtagatgccagggacactgggactctccagctgtgtgtttgtgtcttctgaagact
tatgtttcattcctttggagcatgcataatcatacactgtgggatgtgttatatagattgcttgatagttcacc
actgtaataaaatactgtgactggaatctgctcccagtctgcctttgatagcacttgtgcaacacacatttact
gagcatttacagtgatccaggacctgtgttgtgaaaacattgatggacaaggcagatggtggagcacgtcagtg
aggatttttaacaaaggctggtaagtgctataaaggaacattgtaggacactagagaacaaagaacaggagaac
ctgacttaggctggggtggggcgttggttagaggaggctccttggaggacatgaggtttaagctgtgacctgag
gatgaatagatgttggccaggtgaggtaccggtatttgtcagccttaccagtaaaaaagaaaacctattaaaaa
aaaaatacacatacaaagcctcatcagecatggcttaccagagaaagtacagcgggcacacaaaccacaagctc
taaagtcactctccaacctctccacaatatatatacacaagccctaaactgacgtaatgggactaaagtgtaaa
aaatcccgccaaacccaacacacaccccgaaactgcgtcaccagggaaaagtacagtttcacttccgcaatccc
aacaagcgtcacttcctctttctcacggtacgtcacatcccattaacttacaacgtcattttcccacggccgcg
ccgccccttttaaccgttaaccccacagccaatcaccacacggcccacactttttaaaatcacctcatttacat
attggcaccattccatctataaggtatattattgatgatg (SEQ ID NO: 192)
CD33 E1 sequence in FIG. 5B
CTGCTGTGGGCAGGTGAGTG (SEQ ID NO: 193)
gRNA E1 (corresponds to DNA of SEQ ID NO: 4):
CCCCUGCUGUGGGCAGGUGAGUG (SEQ ID NO: 194)
gRNA E2 (corresponds to DNA of SEQ ID NO: 193):
CCCCAGUUCAUGGUUACUGGUUC (SEQ ID NO: 195)

1. A method of selectively protecting a cell from an anti-CD33 therapeutic, the method comprising contacting the cell with a base editing system comprising a base editing enzyme and a guide RNA (gRNA), wherein the base editing system inactivates expression of CD33.

2. The method of claim 1, wherein the contacting comprises administering to a system or subject comprising the cell:
a nucleic acid encoding the base editing enzyme and a nucleic acid encoding the gRNA; or
the base editing enzyme and the gRNA.

3. The method of claim 2, wherein the system is an in vitro or ex vivo cell or cell culture.

4. A method of selectively protecting a cell of a human subject from an anti-CD33 agent, the method comprising:
administering to a human subject a viral vector comprising a nucleic acid sequence encoding a base editing system comprising a base editing enzyme and a guide RNA (gRNA), wherein the base editing system inactivates expression of CD33; and
administering to the human subject the anti-CD33 agent.

5. A population of cells comprising a first subpopulation expressing CD33 and a second subpopulation in which CD33 expression is inactivated, wherein one or more cells of the population comprise at least one base editing agent of a base editing system selected from a base editing enzyme and a guide RNA (gRNA), wherein the base editing system inactivates expression of CD33, optionally wherein CD33 expression is inactivated in at least 5%, 6%, 7%, 8%, 9%, 10%, 20%, 30%, 40%, or 50% of cells of the population.

6. A cell comprising a base editing system comprising a base editing enzyme and a guide RNA (gRNA), wherein the base editing system inactivates expression of CD33.

7. A base editing system that inactivates CD33 in a cell, the base editing system comprising a base editing enzyme and a guide RNA (gRNA).

8. A kit comprising a base editing enzyme of a base editing system and a guide RNA (gRNA) of a base editing system, wherein the base editing system inactivates expression of CD33, optionally further comprising an anti-CD33 agent and/or instructions for inactivation of CD33 in one or more cells.

9. The method, population, cell, system, or kit of any one of claims 1-8, wherein the base editing system is engineered to cause a genetic modification that inactivates CD33, wherein the inactivating genetic modification comprises a genetic modification at a splicing site of a nucleic acid encoding CD33, optionally wherein the splicing site is a splicing donor site or a splicing acceptor site, optionally wherein the splicing site is an intron 1 splicing donor site, an exon 2 splicing acceptor site, or an exon 3 splicing acceptor site of a nucleic acid encoding CD33.

10. The method, population, cell, system, or kit of any one of claims 1-8, wherein the base editing system is engineered to cause a genetic modification that inactivates CD33, wherein the inactivating genetic modification comprises introduction of a stop codon within a nucleic acid encoding CD33.

11. The method, population, cell, system, or kit of claim 10, wherein the inactivating genetic modification comprises introduction of a stop codon within exon 2 or exon 3 of a nucleic acid encoding CD33.

12. The method, population, cell, system, or kit of any one of claims 1-9, wherein the gRNA is engineered to cause a CD33-inactivating nucleic acid sequence modification selected from C to T at position 38 (G to A on forward strand, intron 1 splicing donor), C to T at position 481 (G to A on forward strand, intron 2 splicing donor), A to G at position 98 (exon2 splice acceptor), A to G at position 683 (exon3 splice acceptor), or A to G at position 1189 (exon4 splice acceptor) of CD33 (using SEQ ID NO: 15 as a reference).

13. The method, population, cell, system, or kit of any one of claims 1-12, wherein the gRNA is engineered to cause a CD33-inactivating nucleic acid sequence modification encoded by any of SEQ ID NOs: 4, 5, 19, 20, 21, and 22, optionally wherein the nucleic acid sequence modification is A to G at position -113 or A to G at position -175 of CD33.

14. The method, population, cell, system, or kit of any one of claims 1-13, wherein the gRNA has at least 80% sequence identity with a sequence selected from SEQ ID NOs: 4, 5, 19, 20, 21, and 22.

15. The method, population, cell, system, or kit of any one of claims 1-13, wherein the gRNA has at least 90% sequence identity with a sequence selected from SEQ ID NOs: 4, 5, 19, 20, 21, and 22.

16. The method, population, cell, system, or kit of any one of claims 1-15, wherein the base-editing system comprises a cytosine base editing enzyme.

17. The method, population, cell, system, or kit of claim 16, wherein the gRNA is engineered to cause a CD33-inactivating nucleic acid sequence modification selected from C to T at position 38 (G to A on forward strand, intron 1 splicing donor), or C to T at position 481 (G to A on forward strand, intron 2 splicing donor) of CD33 (using SEQ ID NO: 15 as a reference).

18. The method, population, cell, system, or kit of claim 16 or 17, wherein the gRNA is engineered to cause a CD33-inactivating nucleic acid sequence modification encoded by any of SEQ ID NOs: 4, 5, 20, 21, and 22.

19. The method, population, cell, system, or kit of claim 16-18, wherein the gRNA has at least 80% sequence identity with a sequence selected from SEQ ID NOs: 4 and 5.

20. The method, population, cell, system, or kit of claim 16-19, wherein the gRNA has at least 90% sequence identity with a sequence selected from SEQ ID NOs: 4 and 5.

21. The method, population, cell, system, or kit of any one of claims 16-20, wherein the cytosine base-editing enzyme is selected from BE1, BE2, BE3, HF-BE3, BE4, BE4max, BE4-GAM, YE1-BE3, EE-BE3, YE2-BE3, YEE-BE3, VQR-BE3, VRER-BE3, Sa-BE3, SA-BE4, SaBE4-Gam, SaKKH-BE3, Cas12a-BE, Target-AID, Target-AID-NG, xBE3, eA3A-BE3, A3A-BE3, and BE-PLUS, optionally wherein the cytosine base-editing enzyme is BE4max and/or SaBE4-Gam.

22. The method, population, cell, system, or kit of any one of claims 1-15, wherein the base-editing system comprises an adenine base editing enzyme.

23. The method, population, cell, system, or kit of claim 22, wherein the gRNA is engineered to cause a CD33-inactivating nucleic acid sequence modification selected from A to G at position 98 (exon2 splice acceptor), A to G at position 683 (exon3 splice acceptor), or A to G at position 1189 (exon4 splice acceptor) of CD33 (using SEQ ID NO: 15 as a reference).

24. The method, population, cell, system, or kit of claim 22 or 23, wherein the gRNA is engineered to cause a CD33-inactivating nucleic acid sequence modification encoded by any of SEQ ID NOs: 19, 20, 21, and 22.

25. The method, population, cell, system, or kit of claim 22-24, wherein the gRNA has at least 80% sequence identity with a sequence selected from SEQ ID NOs: 19, 20, 21, and 22.

26. The method, population, cell, system, or kit of claim 22-25, wherein the gRNA has at least 90% sequence identity with a sequence selected from SEQ ID NOs: 19, 20, 21, and 22.

27. The method, population, cell, system, or kit of any one of claims 22-26, wherein the adenine base editing enzyme is TadA*-dCas9, TadA-TadA*-Cas9, ABE7.9, ABE 6,3, ABE7.10, and/or ABE8e.

28. The method, population, cell, system, or kit of any one of claims 1-27, wherein the cell or cells are hematopoietic stem cells (HSCs).

29. The method, population, cell, system, or kit of any one of claims 1-27, wherein the cell or cells are hematopoietic stem and progenitor cells (HSPCs).

30. The method, population, cell, system, or kit of any one of claims 1-27, wherein the cell or cells are CD34+ HSCs and/or CD34+CD45RA-CD90+ HSCs.

31. The method, population, cell, system, or kit of any one of claims 1-30, wherein the base editing enzyme and/or gRNA are encoded by a vector. or synthesized in vitro

32. The method, population, cell, system, or kit of any one of claims 1-30, wherein the base editing enzyme and/or gRNA synthesized in vitro.

33. The method, population, cell, system, or kit of claim 31, wherein the vector is a viral vector, optionally wherein the viral vector is an adenoviral vector.

34. The method, population, cell, system, or kit claim 33 wherein the adenoviral vector is a helper dependent adenoviral vector.

35. The method, population, cell, system, or kit claim 33 or 34, wherein the adenoviral vector is a helper-dependent Ad35 viral vector.

36. The method, population, cell, system, or kit of claims 32-35, wherein the vector selectively targets HSCs or HSPCs.

37. The method, population, cell, system, or kit of any one of claims 32-36, wherein the vector further encodes a therapeutic polypeptide and/or further comprises a therapeutic gene.

38. The method, population, cell, system, or kit of claim 37, wherein the therapeutic polypeptide is selected from a checkpoint inhibitor, a gene editing molecule, a chimeric antigen receptor that specifically binds a cellular antigen (e.g. a cancer antigen or a viral antigen), a T-cell receptor that specifically binds a cellular antigen (e.g. a cancer antigen or a viral antigen), γ-globin; soluble CD40; CTLA; Fas L; antibodies to CD4, CD5, CD7, CD52, etc.; antibodies to IL1, IL2, IL6; an antibody to TCR specifically present on autoreactive T cells; IL4; IL10; IL12; IL13; IL1Ra, sIL1RI, sIL1R11; sTNFRI; sTNFRII; antibodies to TNF; P53, PTPN22, and DRB11501/DQB1*0602; globin family genes; WAS; phox; dystrophin; pyruvate kinase (PK); CLN3; ABCD1; arylsulfatase A (ARSA); SFTPB; SFTPC; NLX2.1; ABCA3; GATA1; ribosomal protein genes; TERC; CFTR; LRRK2; PARK2; PARK7; PINK1; SNCA; PSEN1; PSEN2; APP; SOD1; TDP43; FUS; ubiquilin 2; C9ORF72, von Willebrand factor (VWF), FI, FII, FV, FVII, factor VIII (FVIII), FIX, FX, FXI, and/or FXIII, optionally wherein the therapeutic polypeptide is selected from FVIII and/or FIX.

39. The method, population, cell, system, or kit of claim 37, wherein the therapeutic gene is selected from FancA, FancB, FancC, FancD1, FancD2, FancE, FancF, FancG, Fancl, FancJ, FancL, FancM, FancN, FancO, FancP, FancQ, FancR, FancS, FancT, FancU, FancV, FancW, γC, JAK3, IL7RA, RAG1, RAG2, DCLRE1C, PRKDC, LIG4, NHEJ1, CD3D, CD3E, CD3Z, CD3G, PTPRC, ZAP70, LCK, AK2, ADA, PNP, WHN, CHD7, ORAI1, STIM1, CORO1A, CIITA, RFXANK, RFX5, RFXAP, RMRP, DKC1, TERT, TINF2, DCLREIB, SLC46A1, ABLI, AKT1, APC, ARSB, BCL11A, BLC1, BLC6, BRCA1, BRCA2, BRIP1, C46, CAS9, C-CAM, CBFAI, CBL, CCR5, CD19, CDA, C-MYC, CRE, CSCR4, CSFIR, CTS-I, CYB5R3, DCC, DHFR, DLL1, DMD, EGFR, ERBA, ERBB, EBRB2, ETSI, ETS2, ETV6, FCC, FGR, FOX, FUSI, FYN, GALNS, GLB1, GNS, GUSB, HBB, HBD, HBE1, HBG1, HBG2, HCR, HGSNAT, HOXB4, HRAS, HYAL1, ICAM-1, iCaspase, IDUA, IDS, JUN, KLF4, KRAS, LYN, MCC, MDM2, MGMT, MLL, MMACI, MYB, MEN-I, MEN-11, MYC, NAGLU, NANOG, NF-1, NF-2, NKX2.1, NOTCH, OCT4, p16, p21, p27, p57, p73, PALB2, RAD51C, ras, at least one of RPL3 through RPL40, RPLPO, RPLP1, RPLP2, at least one of RPS2 through RPS30, RPSA, SGSH, SLX4, SOX2, VHL, and/or WT-I.

40. A population of cells comprising a first subpopulation expressing CD33 and a second subpopulation in which CD33 expression is inactivated, wherein one or more cells comprises an inactivated CD33 gene comprising a nucleic acid sequence according to one or more of SEQ ID NOs: SEQ ID NOs: 4, 5, 19, 20, 21, or 22, optionally wherein CD33 expression is inactivated in at least 5%, 6%, 7%, 8%, 9%, 10%, 20%, 30%, 40%, or 50% of cells of the population, optionally wherein the cells are HSCs, HSPCs, CD34+ HSCs, and/or CD34+CD45RA-CD90+ HSCs.

41. A cell comprising an inactivated CD33 gene comprising a nucleic acid sequence according to one or more of SEQ ID NOs: SEQ ID NOs: 4, 5, 19, 20, 21, or 22, optionally wherein the cell is an HSC, HSPC, CD34+ HSC, and/or CD34+CD45RA-CD90+ HSC.

42. The method, population, cell, system, or kit of any one of claims 1-41, wherein one or more of the cell or cells is contacted with an anti-CD33 agent.

43. A pharmaceutical formulation comprising a population, cell, system, or kit of any one of claims 5-42 and a pharmaceutically acceptable carrier.

44. A method of treating a subject in need thereof, the method comprising administering to the subject a population, cell, system, kit, or pharmaceutical formulation of any one of claims 5-43.

45. The method of claim 44, wherein the method comprises administering to the subject an anti-CD33 agent.

46. The method of claim 44 or 45, wherein the subject is need of treatment for a primary immune deficiency, a secondary immune deficiency, or a disorder selected from FA, SCID, Pompe disease, Gaucher disease, Fabry disease, Mucopolysaccharidosis type I, familial apolipoprotein E deficiency and atherosclerosis (ApoE), viral infections, and/or cancer.

47. The method of claim 44 or 45, wherein the subject is need of treatment for a hematology condition, optionally wherein the hematology condition is a platelet disorder, a bone marrow failure condition, a red cell disorder, an autoimmune hematology, a primary immunodeficiency, or an inborn error of metabolism.

48. The method of claim 44 or 45, wherein the subject is need of treatment for a hematology condition selected from Bernard-Soulier syndrome, Glanzmann thrombasthenia, Diamond-Blackfan anemia, pyruvate kinase deficiency, acquired thrombotic thrombocytopenic purpura (aTTP), congenital thrombotic thrombocytopenic purpura (cTTP), Wiskott-Aldrich syndrome (WAS), Severe combined immunodeficiency due to adenosine deaminase deficiency (ADA-SCID), X-linked severe combined immunodeficiency (SCID-X1), DOCK 8 deficiency, major histocompatibility complex class II deficiency (MHC-II), CD40/CD40L deficiency, hereditary hemochromatosis, and phenylketonuria (PKU).

49. The method, population, cell, system, or kit of any one of claims 42-48, wherein the anti-CD33 agent comprises an anti-CD33 antibody, an anti-CD33 immunotoxin, an anti-CD33 antibody-drug conjugate, an anti-CD33 antibody-radioisotope conjugate, an anti-CD33 bispecific antibody, an anti-CD33 bispecific immune cell engaging antibody, an anti-CD33 trispecific antibody, an anti-CD33 chimeric antigen receptor (CAR) with one or more binding domains, hp67.6, lintuzumab, SGN-CD33A, and/or AMG 330.

50. The method, population, cell, system, or kit of any one of claims 42-48, wherein the anti-CD33 agent comprises a binding domain derived from hp67.6, lintuzumab, SGN-CD33A, and/or AMG 330, wherein the anti-CD33 agent comprises one or more, or all, CDRs of hp67.6, lintuzumab, SGN-CD33A, and/or AMG 330, and/or wherein the anti-CD33 agent comprises a bispecific antibody comprising a combination of binding variable chains or a binding CDR combination of hp67.6, lintuzumab, SGN-CD33A, and/or AMG 330.

51. The method, population, cell, system, or kit of any one of claims 42-48, wherein the anti-CD33 agent comprises an antibody-drug conjugate or an antibody-radioisotope conjugate wherein the drug or radioisotope are selected from taxol, taxane, cytochalasin B, gramicidin D, ethidium bromide, emetine, mitomycin, etoposide, tenoposide, vincristine, vinblastine, colchicin, doxorubicin, daunorubicin, dihydroxy anthracinedione, mitoxantrone, mithramycin, maytansinoid, dolastatin, auristatin, calicheamicin, pyrrolobenzodiazepine, nemorubicin PNU-159682, anthracycline, vinca alkaloid, trichothecene, CC1065, camptothecin, elinafide, actinomycin D, 1-dehydrotestosterone, glucocorticoids, procaine, tetracaine, lidocaine, propranolol, puromycin, ricin, CC-1065, duocarmycin, diphtheria toxin, snake venom, cobra venom, mistletoe lectin, modeccin, pokeweed antiviral protein, saporin, Bryodin 1, bouganin, gelonin, Pseudomonas exotoxin, iodine-131, indium-111, yttrium-90, lutetium-177, astatine-211, bismuth-212, and/or bismuth-213 and/or wherein the antibody-drug conjugate comprises GO.

52. The method, population, cell, system, or kit of any one of claims 42-48, wherein the CD33-targeting agent comprises a bispecific antibody comprising at least one binding domain that activates an immune cell, optionally wherein the immune cell is a T-cell, natural killer (NK) cell, or a macrophage and//or wherein the binding domain that activates an immune cell binds CD3, CD28, CD8, NKG2D, CD8, CD16, KIR2DL4, KIR2DS1, KIR2DS2, KIR3DS1, NKG2C, NKG2E, NKG2D, NKp30, NKp44, NKp46, NKp80, DNAM-1, CD11b, CD11c, CD64, CD68, CD119, CD163, CD206, CD209, F4/80, IFGR2, Toll-like receptors 1-9, IL-4Rα, or MARCO, optionally wherein the binding domains of the bispecific antibody are joined through a linker.

53. The method, population, cell, system, or kit of any one of claims 42-48, wherein the CD33-targeting agent comprises a chimeric antigen receptor (CAR) comprising a binding domain that specifically binds CD33.

54. The method, population, cell, system, or kit of claim 53, wherein:
the CAR comprises an effector domain selected from 4-1BB, CD3ε, CD3δ, CD3ζ, CD27, CD28, CD79A, CD79B, CARD11, DAP10, FcRα, FcRβ, FcRγ, Fyn, HVEM, ICOS, Lck, LAG3, LAT, LRP, NOTCHI, Wnt, NKG2D, OX40, ROR2, Ryk, SLAMFI, Slp76, pTα, TCRα, TCRβ, TRIM, Zap70, PTCH2, or any combination thereof;
the CAR comprises a cytoplasmic signaling sequence derived from CD3 zeta, FcR gamma, CD3 gamma, CD3 delta, CD3 epsilon, CDS, CD22, CD79a, CD79b, or CD66d;
the CAR comprises an intracellular signaling domain and a costimulatory signaling region, optionally wherein the costimulatory signaling region comprises the intracellular domain of CD27, CD28, 4-1BB, OX40, CD30, CD40, lymphocyte function-associated antigen-1, CD2, CD7, LIGHT, NKG2C, or B7-H3;
the CAR comprises a spacer region; and/or
the CAR comprises a transmembrane domain.

55. The method, population, cell, system, or kit of any one of claims 42-54s wherein the anti-CD33 agent comprises a linker.
